SEQUENCE LISTING (2 sequences)

SEQ ID NO: 1 - DNA, 24 bp, Artificial Sequence
  stem-loop sequence
caaaggctcttttcagagccacca

SEQ ID NO: 2 - RNA, 24 nt, Artificial Sequence
  stem-loop sequence
caaaggcucuuuucagagccacca

(19) United States
(12) Patent Application Publication
(10) Pub. No.: US 2023/0348362 A1
(43) Pub. Date: Nov. 2, 2023

(54) LIPID COMPOUNDS AND LIPID NANOPARTICLE COMPOSITIONS

(71) Applicant: Suzhou Abogen Biosciences Co., Ltd., Suzhou (CN)

(72) Inventors: Bo YING, Suzhou (CN); Xiulian WANG, Suzhou (CN)

(21) Appl. No.: 17/801,477

(22) PCT Filed: Jan. 11, 2022
(86) PCT No.: PCT/CN2022/071251

§ 371 Date: Aug. 22, 2022

Related U.S. Application Data
(60) Provisional application No. 63/140,691, filed on Jan. 22, 2021.

(30) Foreign Application Priority Data
Jan. 14, 2021 (CN) 202110051373.1
Oct. 8, 2021 (WO) PCT/CN2021/122704

(51) Int. Cl. C07C 229/46; C07C 237/14; C07D 307/22; C07D 213/38; C07D 335/02; C07F 9/24; C07C 271/20; C07D 211/58; C07D 305/08; C07D 205/04; C07D 207/09; A61K 45/06; A61K 9/51

(52) U.S. Cl. CPC C07C 229/46; C07C 237/14; C07D 307/22; C07D 213/38; C07D 335/02; C07F 9/2458; C07C 271/20; C07D 211/58; C07D 305/08; C07D 205/04; C07D 207/09; A61K 45/06; A61K 9/5123; C07C 2601/02; C07C 2601/04; C07C 2601/08; C07C 2601/14; C07C 2601/18; B82Y 5/00

(57) ABSTRACT

Provided herein are lipid compounds that can be used in combination with other lipid components, such as neutral lipids, cholesterol and polymer conjugated lipids, to form lipid nanoparticles for delivery of therapeutic agents (e.g., nucleic acid molecules) for therapeutic or prophylactic purposes, including vaccination. Also provided herein are lipid nanoparticle compositions comprising said lipid compounds.

### 1. SEQUENCE LISTING

The present specification is being filed with a computer readable form (CRF) copy of the Sequence Listing. The CRF entitled 14639-019-146_SeqListing_ST25.txt, which was created on Dec. 20, 2021 and is 627 bytes in size, and is incorporated herein by reference in its entirety.

### 2. FIELD

The present disclosure generally relates to lipid compounds that can be used in combination with other lipid components, such as neutral lipids, cholesterol and polymer conjugated lipids, to form lipid nanoparticles for delivery of therapeutic agents (e.g., nucleic acid molecules, including nucleic acid mimics such as locked nucleic acids (LNAs), peptide nucleic acids (PNAs), and morpholinos), both in vitro and in vivo, for therapeutic or prophylactic purposes, including vaccination.

### 3. BACKGROUND

Therapeutic nucleic acids have the potential to revolutionize vaccination, gene therapies, protein replacement therapies, and other treatments of genetic diseases. Since the commencement of the first clinical studies on therapeutic nucleic acids in the 2000s, significant progresses have been made through the design of nucleic acid molecules and delivery methods thereof. However, nucleic acid therapeutics still face several challenges, including low cell permeability and high susceptibility to degradation of certain nucleic acids molecules, including RNAs. Thus, there exists a need to develop new nucleic acid molecules, as well as related methods and compositions that facilitate their delivery in vitro or in vivo for therapeutic and/or prophylactic purposes. Lipid compounds that can be used in combination with other lipid components, such as neutral lipids, cholesterol and polymer conjugated lipids, to form lipid nanoparticles for delivery of therapeutic agents. There exists a need to develop new lipid compounds (e.g., cationic lipid compounds) that afford efficient delivery of the therapeutic agents, sufficient activity of the therapeutic agents (e.g., expression of mRNA after delivery), optimal pharmacokinetics, and/or other suitable physiological, biological, and/or therapeutic properties.

### 4. SUMMARY

In one embodiment, provided herein are lipid compounds, including pharmaceutically acceptable salts, prodrugs or stereoisomers thereof, which can be used alone or in combination with other lipid components such as neutral lipids, charged lipids, steroids (including for example, all sterols) and/or their analogs, and/or polymer conjugated lipids and/or polymers to form lipid nanoparticles for the delivery of therapeutic agents (e.g., nucleic acid molecules, including nucleic acid mimics such as locked nucleic acids (LNAs), peptide nucleic acids (PNAs), and morpholinos). In some instances, the lipid nanoparticles are used to deliver nucleic acids such as antisense and/or messenger RNA. Methods for use of such lipid nanoparticles for treatment of various diseases or conditions, such as those caused by infectious entities and/or insufficiency of a protein, are also provided.

In one embodiment, provided herein is a compound of Formula (I):

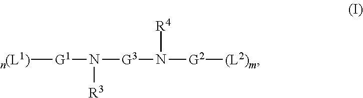

or a pharmaceutically acceptable salt, prodrug or stereoisomer thereof, wherein G¹, G², G³, L¹, L², R³, R⁴, n, and m are as defined herein or elsewhere.

In one embodiment, provided herein is a nanoparticle composition comprising a compound provided herein, and a therapeutic or prophylactic agent. In one embodiment, the therapeutic or prophylactic agent comprises at least one mRNA encoding an antigen or a fragment or epitope thereof.

Additional features of the present disclosure will become apparent to those skilled in the art upon consideration of the following detailed description of particular embodiments.

### 5. DETAILED DESCRIPTION

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein and unless otherwise specified, the term “lipid” refers to a group of organic compounds that include, but are not limited to, esters of fatty acids and are generally characterized by being poorly soluble in water, but soluble in many nonpolar organic solvents. While lipids generally have poor solubility in water, there are certain categories of lipids (e.g., lipids modified by polar groups, e.g., DMG-PEG2000) that have limited aqueous solubility and can dissolve in water under certain conditions. Known types of lipids include biological molecules such as fatty acids, waxes, sterols, fat-soluble vitamins, monoglycerides, diglycerides, triglycerides, and phospholipids. Lipids can be divided into at least three classes: (1) “simple lipids,” which include fats and oils as well as waxes; (2) “compound lipids,” which include phospholipids and glycolipids (e.g., DMPE-PEG2000); and (3) “derived lipids” such as steroids. Further, as used herein, lipids also encompass lipidoid compounds. The term “lipidoid compound,” also simply “lipidoid”, refers to a lipid-like compound (e.g. an amphiphilic compound with lipid-like physical properties).

The term “lipid nanoparticle” or “LNP” refers to a particle having at least one dimension on the order of nanometers (nm) (e.g., 1 to 1,000 nm), which contains one or more types of lipid molecules. The LNP provided herein can further contain at least one non-lipid payload molecule (e.g., one or more nucleic acid molecules). In some embodiments, the LNP comprises a non-lipid payload molecule either partially or completely encapsulated inside a lipid shell. Particularly, in some embodiments, wherein the payload is a negatively charged molecule (e.g., mRNA encoding a viral protein), and the lipid components of the LNP comprise at least one cationic lipid. Without being bound by the theory, it is contemplated that the cationic lipids can interact with the negatively charged payload molecules and facilitates incorporation and/or encapsulation of the payload into the LNP during LNP formation. Other lipids that can form part of a LNP as provided herein include but are not limited to neutral lipids and charged lipids, such as steroids, polymer conjugated lipids, and various zwitterionic lipids. In certain embodiments, a LNP according to the present disclosure comprises one or more lipids of Formula (I) (and sub-formulas thereof) as described herein.

The term “cationic lipid” refers to a lipid that is either positively charged at any pH value or hydrogen ion activity of its environment, or capable of being positively charged in response to the pH value or hydrogen ion activity of its environment (e.g., the environment of its intended use). Thus, the term “cationic” encompasses both “permanently cationic” and “cationisable.” In certain embodiments, the positive charge in a cationic lipid results from the presence of a quaternary nitrogen atom. In certain embodiments, the cationic lipid comprises a zwitterionic lipid that assumes a positive charge in the environment of its intended use (e.g., at physiological pH). In certain embodiments, the cationic lipid is one or more lipids of Formula (I) (and sub-formulas thereof) as described herein.

The term “polymer conjugated lipid” refers to a molecule comprising both a lipid portion and a polymer portion. An example of a polymer conjugated lipid is a pegylated lipid (PEG-lipid), in which the polymer portion comprises a polyethylene glycol.

The term “neutral lipid” encompasses any lipid molecules existing in uncharged forms or neutral zwitterionic forms at a selected pH value or within a selected pH range. In some embodiments, the selected useful pH value or range corresponds to the pH condition in an environment of the intended uses of the lipids, such as the physiological pH. As non-limiting examples, neutral lipids that can be used in connection with the present disclosure include, but are not limited to, phosphotidylcholines such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), 2-((2,3-bis(oleoyloxy)propyl)dimethylammonio)ethyl hydrogen phosphate (DOCP), sphingomyelins (SM), ceramides, steroids such as sterols and their derivatives. Neutral lipids as provided herein may be synthetic or derived (isolated or modified) from a natural source or compound.

The term “charged lipid” encompasses any lipid molecules that exist in either positively charged or negatively charged forms at a selected pH or within a selected pH range. In some embodiments, the selected pH value or range corresponds to the pH condition in an environment of the intended uses of the lipids, such as the physiological pH. As non-limiting examples, charged lipids that can be used in connection with the present disclosure include, but are not limited to, phosphatidylserines, phosphatidic acids, phosphatidylglycerols, phosphatidylinositols, sterol hemisuccinates, dialkyl trimethylarnmonium-propanes, (e.g., DOTAP, DOTMA), dialkyl dimethylaminopropanes, ethyl phosphocholines, dimethylaminoethane carbamoyl sterols (e.g., DC-Chol), 1,2-dioleoyl-sn-glycero-3-phospho-L-serine sodium salt (DOPS-Na), 1,2-dioleoyl-sn-glycero-3-phospho-(1′-rac-glycerol) sodium salt (DOPG-Na), and 1,2-dioleoyl-sn-glycero-3-phosphate sodium salt (DOPA-Na). Charged lipids as provided herein may be synthetic or derived (isolated or modified) from a natural source or compound.

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, and the like. Unless otherwise specified, an alkenyl group is optionally substituted.

As used herein, and unless otherwise specified, the term “alkynyl” refers to a straight or branched hydrocarbon chain radical consisting solely of carbon and hydrogen atoms, which contains one or more carbon-carbon triple bonds. In one embodiment, the alkynyl group has, for example, from two to twenty-four carbon atoms (C₂-C₂₄ alkynyl), four to twenty carbon atoms (C₄-C₂₀ alkynyl), six to sixteen carbon atoms (C₆-C₁₆ alkynyl), six to nine carbon atoms (C₆-C₉ alkynyl), two to fifteen carbon atoms (C₂-C₁₅ alkynyl), two to twelve carbon atoms (C₂-C₁₂ alkynyl), two to eight carbon atoms (C₂-C₈ alkynyl) or two to six carbon atoms (C₂-C₆ alkynyl) and which is attached to the rest of the molecule by a single bond. Examples of alkynyl groups include, but are not limited to, ethynyl, propynyl, butynyl, pentynyl, and the like. Unless otherwise specified, an alkynyl group is optionally substituted.

As used herein, and unless otherwise specified, the term “alkylene” or “alkylene chain” refers to a straight or branched multivalent (e.g., divalent or trivalent) hydrocarbon chain linking the rest of the molecule to a radical group (or groups), consisting solely of carbon and hydrogen, which is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C1-C₂ alkylene). Examples of alkylene groups include, but are not limited to, methylene, ethylene, propylene, n-butylene, and the like. The alkylene chain is attached to the rest of the molecule through a single bond and to the radical group through a single bond. The points of attachment of the alkylene chain to the rest of the molecule and to the radical group(s) can be through one carbon or any two (or more) carbons within the chain. Unless otherwise specified, an alkylene chain is optionally substituted.

As used herein, and unless otherwise specified, the term “alkenylene” refers to a straight or branched multivalent (e.g., divalent or trivalent) hydrocarbon chain linking the rest of the molecule to a radical group (or groups), consisting solely of carbon and hydrogen, which contains one or more carbon-carbon double bonds. In one embodiment, the alkenylene has, for example, from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₅ alkenylene), two to six carbon atoms (C₂-C₆ alkenylene) or two to four carbon atoms (C₂-C₄ alkenylene). Examples of alkenylene include, but are not limited to, ethenylene, propenylene, n-butenylene, and the like. The alkenylene is attached to the rest of the molecule through a single or double bond and to the radical group through a single or double bond. The points of attachment of the alkenylene to the rest of the molecule and to the radical group(s) can be through one carbon or any two (or more) carbons within the chain. Unless otherwise specified, an alkenylene is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused or bridged ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooctyl. Examples of polycyclic cycloalkyl radicals include, but are not limited to, adamantyl, norbornyl, decalinyl, 7,7-dimethyl-bicyclo[2.2.1]heptanyl, and the like. Unless otherwise specified, a cycloalkyl group is optionally substituted.

As used herein, and unless otherwise specified, the term “cycloalkylene” is a multivalent (e.g., divalent or trivalent) cycloalkyl group. Unless otherwise specified, a cycloalkylene group is optionally substituted.

As used herein, and unless otherwise specified, the term “cycloalkenyl” refers to a non-aromatic monocyclic or polycyclic hydrocarbon radical consisting solely of carbon and hydrogen atoms, and which includes one or more carbon-carbon double bonds. Cycloalkenyl may include fused or bridged ring systems. In one embodiment, the cycloalkenyl has, for example, from 3 to 15 ring carbon atoms (C₃-C₁₅ cycloalkenyl), from 3 to 10 ring carbon atoms (C₃-C₁₀ cycloalkenyl), or from 3 to 8 ring carbon atoms (C₃-C₈ cycloalkenyl). The cycloalkenyl is attached to the rest of the molecule by a single bond. Examples of monocyclic cycloalkenyl radicals include, but are not limited to, cyclopropenyl, cyclobutenyl, cyclopentenyl, cyclohexenyl, cycloheptenyl, cyclooctenyl, and the like. Unless otherwise specified, a cycloalkenyl group is optionally substituted.

As used herein, and unless otherwise specified, the term “cycloalkenylene” is a multivalent (e.g., divalent or trivalent) cycloalkenyl group. Unless otherwise specified, a cycloalkenylene group is optionally substituted.

As used herein, and unless otherwise specified, the term “heterocyclyl” refers to a non-aromatic radical monocyclic or polycyclic moiety that contains one or more (e.g., one, one or two, one to three, or one to four) heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other polycyclic ring system, wherein the polycyclic ring systems can be a fused, bridged or spiro ring system. Heterocyclyl polycyclic ring systems can include one or more heteroatoms in one or more rings. A heterocyclyl group can be saturated or partially unsaturated. Saturated heterocycloalkyl groups can be termed “heterocycloalkyl”. Partially unsaturated heterocycloalkyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 18 ring atoms (3- to 18-membered heterocyclyl), 4 to 8 ring atoms (4- to 8-membered heterocyclyl), or 5 to 8 ring atoms (5- to 8-membered heterocyclyl). Whenever it appears herein, a numerical range such as “3 to 18” refers to each integer in the given range; e.g., “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Examples of heterocyclyl groups include, but are not limited to, imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl. Unless otherwise specified, a heterocyclyl group is optionally substituted.

As used herein, and unless otherwise specified, the term “heterocyclylene” is a multivalent (e.g., divalent or trivalent) heterocyclyl group. Unless otherwise specified, a heterocyclylene group is optionally substituted.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

As used herein, and unless otherwise specified, the term “arylene” is a multivalent (e.g., divalent or trivalent) aryl group. Unless otherwise specified, an arylene group is optionally substituted.

As used herein, and unless otherwise specified, the term “heteroaryl” refers to a monocyclic aromatic group and/or multicyclic aromatic group that contains at least one aromatic ring, wherein at least one aromatic ring contains one or more (e.g., one, one or two, one to three, or one to four) heteroatoms independently selected from O, S, and N. The heteroaryl may be attached to the main structure at any heteroatom or carbon atom. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrolinyl, acridinyl, phenanthridinyl, and xanthenyl. Unless otherwise specified, a heteroaryl group is optionally substituted.

As used herein, and unless otherwise specified, the term “heteroarylene” is a multivalent (e.g., divalent or trivalent) heteroaryl group. Unless otherwise specified, a heteroarylene group is optionally substituted.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, Cl, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

As used herein, and unless otherwise specified, the term “prodrug” of a biologically active compound refers to a compound that may be converted under physiological conditions or by solvolysis to the biologically active compound. In one embodiment, the term “prodrug” refers to a metabolic precursor of the biologically active compound that is pharmaceutically acceptable. A prodrug may be inactive when administered to a subject in need thereof, but is converted in vivo to the biologically active compound. Prodrugs are typically rapidly transformed in vivo to yield the parent biologically active compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

In one embodiment, the term “prodrug” is also meant to include any covalently bonded carriers, which release the active compound in vivo when such prodrug is administered to a mammalian subject. Prodrugs of a compound may be prepared by modifying functional groups present in the compound in such a way that the modifications are cleaved, either in routine manipulation or in vivo, to the parent compound. Prodrugs include compounds wherein a hydroxyl, amino or mercapto group is bonded to any group that, when the prodrug of the compound is administered to a mammalian subject, cleaves to form a free hydroxyl, free amino or free mercapto group, respectively.

Examples of prodrugs include, but are not limited to, acetate, formate and benzoate derivatives of alcohol or amide derivatives of amine functional groups in the compounds provided herein.

As used herein, and unless otherwise specified, the term “pharmaceutically acceptable salt” includes both acid and base addition salts.

Examples of pharmaceutically acceptable acid addition salts include, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Examples of pharmaceutically acceptable base addition salt include, but are not limited to, salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

A compound provided herein may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Unless otherwise specified, a compound provided herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

It should also be noted a compound described herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (2H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound described herein, whether radioactive or not, are intended to be encompassed within the scope of the embodiments provided herein. In some embodiments, there are provided isotopologs of a compound described herein, for example, the isotopologs are deuterium, carbon-13, and/or nitrogen-15 enriched. As used herein, “deuterated”, means a compound wherein at least one hydrogen (H) has been replaced by deuterium (indicated by D or 2H), that is, the compound is enriched in deuterium in at least one position.

It should be noted that if there is a discrepancy between a depicted structure and a name for that structure, the depicted structure is to be accorded more weight.

As used herein, and unless otherwise specified, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “composition” is intended to encompass a product containing the specified ingredients (e.g., a mRNA molecule provided herein) in, optionally, the specified amounts.

The term “polynucleotide” or “nucleic acid,” as used interchangeably herein, refers to polymers of nucleotides of any length and includes, e.g., DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. Nucleic acid can be in either single- or double-stranded forms. As used herein and unless otherwise specified, “nucleic acid” also includes nucleic acid mimics such as locked nucleic acids (LNAs), peptide nucleic acids (PNAs), and morpholinos. “Oligonucleotide,” as used herein, refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as an mRNA molecule, can be substantially free of other cellular material, or culture medium when produced by recombinant techniques, or substantially free of chemical precursors or other chemicals when chemically synthesized. In a specific embodiment, one or more nucleic acid molecules encoding an antigen as described herein are isolated or purified. The term embraces nucleic acid sequences that have been removed from their naturally occurring environment, and includes recombinant or cloned DNA or RNA isolates and chemically synthesized analogues or analogues biologically synthesized by heterologous systems. A substantially pure molecule may include isolated forms of the molecule.

The term “encoding nucleic acid” or grammatical equivalents thereof as it is used in reference to nucleic acid molecule encompasses (a) a nucleic acid molecule in its native state or when manipulated by methods well known to those skilled in the art that can be transcribed to produce mRNA which is then translated into a peptide and/or polypeptide, and (b) the mRNA molecule itself. The antisense strand is the complement of such a nucleic acid molecule, and the encoding sequence can be deduced therefrom. The term “coding region” refers to a portion in an encoding nucleic acid sequence that is translated into a peptide or polypeptide. The term “untranslated region” or “UTR” refers to the portion of an encoding nucleic acid that is not translated into a peptide or polypeptide. Depending on the orientation of a UTR with respect to the coding region of a nucleic acid molecule, a UTR is referred to as the 5′-UTR if located to the 5′-end of a coding region, and a UTR is referred to as the 3′-UTR if located to the 3′-end of a coding region.

The term “mRNA” as used herein refers to a message RNA molecule comprising one or more open reading frame (ORF) that can be translated by a cell or an organism provided with the mRNA to produce one or more peptide or protein product. The region containing the one or more ORFs is referred to as the coding region of the mRNA molecule. In certain embodiments, the mRNA molecule further comprises one or more untranslated regions (UTRs).

In certain embodiments, the mRNA is a monocistronic mRNA that comprises only one ORF. In certain embodiments, the monocistronic mRNA encodes a peptide or protein comprising at least one epitope of a selected antigen (e.g., a pathogenic antigen or a tumor associated antigen). In other embodiments, the mRNA is a multicistronic mRNA that comprises two or more ORFs. In certain embodiments, the multiecistronic mRNA encodes two or more peptides or proteins that can be the same or different from each other. In certain embodiments, each peptide or protein encoded by a multicistronic mRNA comprises at least one epitope of a selected antigen. In certain embodiments, different peptide or protein encoded by a multicistronic mRNA each comprises at least one epitope of different antigens. In any of the embodiments described herein, the at least one epitope can be at least 2, at least 3, at least 4, at least 5, at least 6, at least 7, at least 8, at least 9, or at least 10 epitopes of an antigen.

The term “nucleobases” encompasses purines and pyrimidines, including natural compounds adenine, thymine, guanine, cytosine, uracil, inosine, and natural or synthetic analogs or derivatives thereof.

The term “functional nucleotide analog” as used herein refers to a modified version of a canonical nucleotide A, G, C, U or T that (a) retains the base-pairing properties of the corresponding canonical nucleotide, and (b) contains at least one chemical modification to (i) the nucleobase, (ii) the sugar group, (iii) the phosphate group, or (iv) any combinations of (i) to (iii), of the corresponding natural nucleotide. As used herein, base pairing encompasses not only the canonical Watson-Crick adenine-thymine, adenine-uracil, or guanine-cytosine base pairs, but also base pairs formed between canonical nucleotides and functional nucleotide analogs or between a pair of functional nucleotide analogs, wherein the arrangement of hydrogen bond donors and hydrogen bond acceptors permits hydrogen bonding between a modified nucleobase and a canonical nucleobase or between two complementary modified nucleobase structures. For example, a functional analog of guanosine (G) retains the ability to base-pair with cytosine (C) or a functional analog of cytosine. One example of such non-canonical base pairing is the base pairing between the modified nucleotide inosine and adenine, cytosine, or uracil. As described herein, a functional nucleotide analog can be either naturally occurring or non-naturally occurring. Accordingly, a nucleic acid molecule containing a functional nucleotide analog can have at least one modified nucleobase, sugar group and/or internucleoside linkage. Exemplary chemical modifications to the nucleobases, sugar groups, or internucleoside linkages of a nucleic acid molecule are provided herein.

The terms “translational enhancer element,” “TEE” and “translational enhancers” as used herein refers to an region in a nucleic acid molecule that functions to promotes translation of a coding sequence of the nucleic acid into a protein or peptide product, such as via cap-dependent or cap-independent translation. A TEE typically locates in the UTR region of a nucleic acid molecule (e.g., mRNA) and enhance the translational level of a coding sequence located either upstream or downstream. For example, a TEE in a 5′-UTR of a nucleic acid molecule can locate between the promoter and the starting codon of the nucleic acid molecule. Various TEE sequences are known in the art (Wellensiek et al. Genome-wide profiling of human cap-independent translation-enhancing elements, Nature Methods, 2013 August; 10(8): 747-750; Chappell et al. PNAS Jun. 29, 2004 101 (26) 9590-9594). Some TEEs are known to be conserved across multiple species (Pánek et al. Nucleic Acids Research, Volume 41, Issue 16, 1 Sep. 2013, Pages 7625-7634).

As used herein, the term “stem-loop sequence” refers to a single-stranded polynucleotide sequence having at least two regions that are complementary or substantially complementary to each other when read in opposite directions, and thus capable of base-pairing with each other to form at least one double helix and an unpaired loop. The resulting structure is known as a stem-loop structure, a hairpin, or a hairpin loop, which is a secondary structure found in many RNA molecules.

The term “peptide” as used herein refers to a polymer containing between two and fifty (2-50) amino acid residues linked by one or more covalent peptide bond(s). The terms apply to naturally occurring amino acid polymers as well as amino acid polymers in which one or more amino acid residues is a non-naturally occurring amino acid (e.g., an amino acid analog or non-natural amino acid).

The terms “polypeptide” and “protein” are used interchangeably herein to refer to a polymer of greater than fifty (50) amino acid residues linked by covalent peptide bonds. That is, a description directed to a polypeptide applies equally to a description of a protein, and vice versa. The terms apply to naturally occurring amino acid polymers as well as amino acid polymers in which one or more amino acid residues is a non-naturally occurring amino acid (e.g., an amino acid analog). As used herein, the terms encompass amino acid chains of any length, including full length proteins (e.g., antigens).

The term “antigen” refers to a substance that can be recognized by the immune system of a subject (including by the adaptive immune system), and is capable of triggering an immune response after the subject is contacted with the antigen (including an antigen-specific immune response). In certain embodiments, the antigen is a protein associated with a diseased cell, such as a cell infected by a pathogen or a neoplastic cell (e.g., tumor associated antigen (TAA)).

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, fragments refers to polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a polypeptide. In a specific embodiment, a fragment of a polypeptide retains at least 1, at least 2, at least 3, or more functions of the polypeptide.

An “epitope” is the site on the surface of an antigen molecule to which a single antibody molecule binds, such as a localized region on the surface of an antigen that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. In certain embodiments, an epitope is a three-dimensional surface feature of a polypeptide. In other embodiments, an epitope is linear feature of a polypeptide. Generally an antigen has several or many different epitopes and may react with many different antibodies.

The term “genetic vaccine” as used herein refers to a therapeutic or prophylactic composition comprising at least one nucleic acid molecule encoding an antigen associated with a target disease (e.g., an infectious disease or a neoplastic disease). Administration of the vaccine to a subject (“vaccination”) allows for the production of the encoded peptide or protein, thereby eliciting an immune response against the target disease in the subject. In certain embodiments, the immune response comprises adaptive immune response, such as the production of antibodies against the encoded antigen, and/or activation and proliferations of immune cells capable of specifically eliminating diseased cells expressing the antigen. In certain embodiments, the immune response further comprises innate immune response. According to the present disclosure, a vaccine can be administered to a subject either before or after the onset of clinical symptoms of the target disease. In some embodiments, vaccination of a healthy or asymptomatic subject renders the vaccinated subject immune or less susceptible to the development of the target disease. In some embodiments, vaccination of a subject showing symptoms of the disease improves the condition of, or treats, the disease in the vaccinated subject.

The terms “innate immune response” and “innate immunity” are recognized in the art, and refer to non-specific defense mechanism a body's immune system initiates upon recognition of pathogen-associated molecular patterns, which involves different forms of cellular activities, including cytokine production and cell death through various pathways. As used herein, innate immune responses include, without limitation, increased production of inflammation cytokines (e.g., type I interferon or IL-10 production), activation of the NFκB pathway, increased proliferation, maturation, differentiation and/or survival of immune cells, and in some cases, induction of cell apoptosis. Activation of the innate immunity can be detected using methods known in the art, such as measuring the (NF)-κB activation.

The terms “adaptive immune response” and “adaptive immunity” are recognized in the art, and refer to antigen-specific defense mechanism a body's immune system initiates upon recognition of a specific antigen, which include both humoral response and cell-mediated responses. As used herein, adaptive immune responses include cellular responses that is triggered and/or augmented by a vaccine composition, such as a genetic composition described herein. In some embodiments, the vaccine composition comprises an antigen that is the target of the antigen-specific adaptive immune response. In other embodiments, the vaccine composition, upon administration, allows the production in an immunized subject of an antigen that is the target of the antigen-specific adaptive immune response. Activation of an adaptive immune response can be detected using methods known in the art, such as measuring the antigen-specific antibody production, or the level of antigen-specific cell-mediated cytotoxicity.

The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide, a fragment or an epitope thereof. Antibodies also include, but are not limited to, synthetic antibodies, recombinantly produced antibodies, camelized antibodies, intrabodies, anti-idiotypic (anti-Id) antibodies, and functional fragments of any of the above, which refers to a portion of an antibody heavy or light chain polypeptide that retains some or all of the binding activity of the antibody from which the fragment was derived. Non-limiting examples of functional fragments include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “administer” or “administration” refers to the act of injecting or otherwise physically delivering a substance as it exists outside the body (e.g., a lipid nanoparticle composition as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

“Chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

The term “targeted delivery” or the verb form “target” as used herein refers to the process that promotes the arrival of a delivered agent (such as a therapeutic payload molecule in a lipid nanoparticle composition as described herein) at a specific organ, tissue, cell and/or intracellular compartment (referred to as the targeted location) more than any other organ, tissue, cell or intracellular compartment (referred to as the non-target location). Targeted delivery can be detected using methods known in the art, for example, by comparing the concentration of the delivered agent in a targeted cell population with the concentration of the delivered agent at a non-target cell population after systemic administration. In certain embodiments, targeted delivery results in at least 2 fold higher concentration at a targeted location as compared to a non-target location.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including, for example, infection and neoplasia. In some embodiments, the effective amount is a therapeutically effective amount or a prophylactically effective amount.

The term “therapeutically effective amount” as used herein refers to the amount of an agent (e.g., a vaccine composition) that is sufficient to reduce and/or ameliorate the severity and/or duration of a given disease, disorder, or condition, and/or a symptom related thereto (e.g., an infectious disease such as caused by viral infection, or a neoplastic disease such as cancer). A “therapeutically effective amount” of a substance/molecule/agent of the present disclosure (e.g., the lipid nanoparticle composition as described herein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a lipid nanoparticle composition as described herein or a therapeutic or prophylactic agent contained therein (e.g., a therapeutic mRNA) effective to “treat” a disease, disorder, or condition, in a subject or mammal.

A “prophylactically effective amount” is an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s) (e.g., an infectious disease such as caused by viral infection, or a neoplastic disease such as cancer). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

The terms “prevent,” “preventing,” and “prevention” refer to reducing the likelihood of the onset (or recurrence) of a disease, disorder, condition, or associated symptom(s) (e.g., an infectious disease such as caused by viral infection, or a neoplastic disease such as cancer).

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as a lipid nanoparticle composition as described herein) to “manage” an infectious or neoplastic disease, one or more symptoms thereof, so as to prevent the progression or worsening of the disease.

The term “prophylactic agent” refers to any agent that can totally or partially inhibit the development, recurrence, onset, or spread of disease and/or symptom related thereto in a subject.

The term “therapeutic agent” refers to any agent that can be used in treating, preventing, or alleviating a disease, disorder, or condition, including in the treatment, prevention, or alleviation of one or more symptoms of a disease, disorder, or condition and/or a symptom related thereto.

The term “therapy” refers to any protocol, method, and/or agent that can be used in the prevention, management, treatment, and/or amelioration of a disease, disorder, or condition. In certain embodiments, the terms “therapies” and “therapy” refer to a biological therapy, supportive therapy, and/or other therapies useful in the prevention, management, treatment, and/or amelioration of a disease, disorder, or condition, known to one of skill in the art such as medical personnel.

As used herein, a “prophylactically effective serum titer” is the serum titer of an antibody in a subject (e.g., a human), that totally or partially inhibits the development, recurrence, onset, or spread of a disease, disorder, or condition, and/or symptom related thereto in the subject.

In certain embodiments, a “therapeutically effective serum titer” is the serum titer of an antibody in a subject (e.g., a human), that reduces the severity, the duration, and/or the symptoms associated with a disease, disorder, or condition, in the subject.

The term “serum titer” refers to an average serum titer in a subject from multiple samples (e.g., at multiple time points) or in a population of at least 10, at least 20, at least 40 subjects, up to about 100, 1000, or more.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal (e.g., a human) having an infectious disease or neoplastic disease. In another embodiment, the subject is a mammal (e.g., a human) at risk of developing an infectious disease or neoplastic disease.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as an antigen encoded by an mRNA molecule as described herein, in a sample or subject. A detectable agent can be a substance that is capable of being visualized or a substance that is otherwise able to be determined and/or measured (e.g., by quantitation).

“Substantially all” refers to at least about 60%, at least about 65%, at least about 70%, at least about 75%, at least about 80%, at least about 85%, at least about 90%, at least about 95% at least about 98%, at least about 99%, or about 100%.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, 0.05%, or less of a given value or range.

The singular terms “a,” “an,” and “the” as used herein include the plural reference unless the context clearly indicates otherwise.

All publications, patent applications, accession numbers, and other references cited in this specification are herein incorporated by reference in their entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section and examples are intended to illustrate but not limit the scope of invention described in the claims.

### 5.3 Lipid Compounds

Unless otherwise specified, the descriptions provided herein apply to all the formulas provided herein (e.g., Formula (I), including their sub-formulas), to the extent that they are applicable.

In one embodiment, provided herein is a compound of Formula (I):

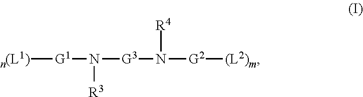

or a pharmaceutically acceptable salt, prodrug or stereoisomer thereof, wherein:
G¹ and G² are each independently a bond, C₂-C₁₂ alkylene, or C₂-C₁₂ alkenylene, wherein one or more —CH₂— in G¹ and G² is optionally replaced by —O—;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c}), —(C₆-C₁₀ arylene)-R¹, -(6- to 10-membered heteroarylene)-R¹, -(4- to 8-membered heterocyclylene)-R¹, or R¹;
each L² is independently —OC(═O)R², —C(═O)OR², —OC(═O)OR², —C(═O)R², —OR², —S(O)ₓR², —S—SR², —C(═O)SR², —SC(═OC(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f}), —(C₆-C₁₀ arylene)-R², -(6- to 10-membered heteroarylene)-R², -(4- to 8-membered heterocyclylene)-R², or R²;
R¹ and R² are each independently C₆-C₂₄ alkyl or C₆-C₂₄ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety; or R³, G³ or part of G³, together with the nitrogen to which they are attached form a cyclic moiety;
R⁴ is C₁-C₁₂ alkyl or C₃-C₈ cycloalkyl;
x is 0, 1, or 2;
n is 1 or 2;
m is 1 or 2; and
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

In one embodiment, provided herein is a compound of Formula (I):

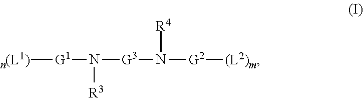

or a pharmaceutically acceptable salt, prodrug or stereoisomer thereof, wherein:
G¹ and G² are each independently a bond, C₂-C₁₂ alkylene, or C₂-C₁₂ alkenylene;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}(═O)OR², —SC(═S)R², —C(═S)SR², —C(═S)R², —CH(OH)R², —P(═O)(OR^{e})(OR^{f}), —(C₆-C₁₀ arylene)-R², -(6- to 10-membered heteroarylene)-R², or R²;
R¹ and R² are each independently C₆-C₂₄ alkyl or C₆-C₂₄ alkenyl;
R^{a}, R^{b}, R^{d}, and R^{e} are each independently H, C₁-C₁₂ alkyl, or C₂-C₁₂ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety; or R³, G³ or part of G³, together with the nitrogen to which they are attached form a cyclic moiety;
R⁴ is C₁-C₁₂ alkyl or C₃-C₈ cycloalkyl;
x is 0, 1, or 2;
n is 1 or 2;
m is 1 or 2; and
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

In one embodiment, n is 1. In one embodiment, n is 2. In one embodiment, m is 1. In one embodiment, m is 2. In one embodiment, n is 1 and m is 1. In one embodiment, n is 1 and m is 2. In one embodiment, n is 2 and m is 1. In one embodiment, n is 2 and m is 2.

In one embodiment, the compound is a compound of Formula (II-A):

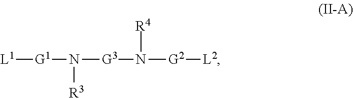

or a pharmaceutically acceptable salt, prodrug or stereoisomer thereof.

In one embodiment, the compound is a compound of Formula (II-B):

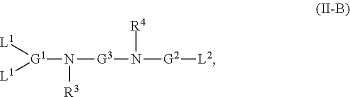

or a pharmaceutically acceptable salt, prodrug or stereoisomer thereof.

In one embodiment, the compound is a compound of Formula (II-C):

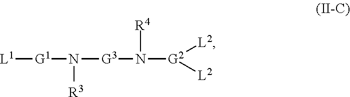

or a pharmaceutically acceptable salt, prodrug or stereoisomer thereof.

In one embodiment, the compound is a compound of Formula (II-D):

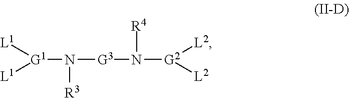

or a pharmaceutically acceptable salt, prodrug or stereoisomer thereof.

In one embodiment, G³ is C₂-C₁₂ alkylene. In one embodiment, G³ is C₂-C₅ alkylene. In one embodiment, G³ is C₂-C₆ alkylene. In one embodiment, G³ is C₂-C₄ alkylene. In one embodiment, G³ is C₂ alkylene. In one embodiment, G³ is C₃ alkylene. In one embodiment, G³ is C₄ alkylene. In one embodiment, G³ is C₅ alkylene. In one embodiment, G³ is C₆ alkylene. In one embodiment, G³ is —CH₂CH₂—.

In one embodiment, G³ is C₂-C₁₂ alkenylene. In one embodiment, G³ is C₂-C₈ alkenylene. In one embodiment, G³ is C₂-C₆ alkenylene. In one embodiment, G³ is C₂-C₄ alkenylene. In one embodiment, G³ is C₂ alkenylene. In one embodiment, G³ is C₃ alkenylene. In one embodiment, G³ is C₄ alkenylene. In one embodiment, G³ is C₅ alkenylene. In one embodiment, G³ is C₆ alkenylene. In one embodiment, G³ is (Z)—CH₂—CH═CH—CH₂—. In one embodiment, G³ is (E)-CH₂—CH═CH—CH₂—.

In one embodiment, G³ is C₂-C₁₂ alkylene or C₂-C₁₂ alkenylene, wherein part or all of alkylene or alkenylene is replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene. In one embodiment, G³ is C₂-C₁₂ alkylene or C₂-C₁₂ alkenylene, wherein part or all of alkylene or alkenylene is replaced by C₃-C₈ cycloalkylene. In one embodiment, G³ is C₂-C₁₂ alkylene or C₂-C₁₂ alkenylene, wherein all of alkylene or alkenylene is replaced by C₃-C₈ cycloalkylene, i.e., G³ is C₃-C₈ cycloalkylene. In one embodiment, G³ is cyclopropylene. In one embodiment, G³ is cyclobutylene. In one embodiment, G³ is cyclopentylene. In one embodiment, G³ is cyclohexylene. In one embodiment, G³ is cycloheptylene. In one embodiment, G³ is cyclooctylene.

In one embodiment, G³ is

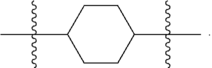 [SMILES: *C1CCC(*)CC1]

In one embodiment, G³ is unsubstituted.

In one embodiment, the compound is a compound of Formula (III-A):

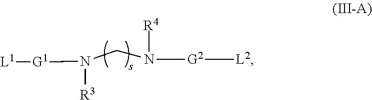

wherein s is an integer from 2 to 12,
   or a pharmaceutically acceptable salt, prodrug or stereoisomer thereof.

In one embodiment, the compound is a compound of Formula (III-B):

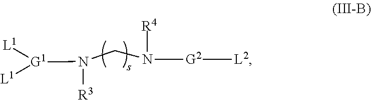

wherein s is an integer from 2 to 12,
   or a pharmaceutically acceptable salt, prodrug or stereoisomer thereof.

In one embodiment, the compound is a compound of Formula (III-C):

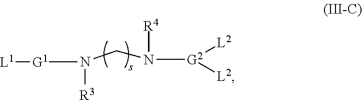

wherein s is an integer from 2 to 12,
   or a pharmaceutically acceptable salt, prodrug or stereoisomer thereof.

In one embodiment, the compound is a compound of Formula (III-D):

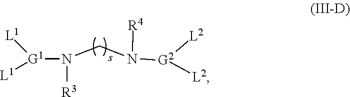

wherein s is an integer from 2 to 12,
   or a pharmaceutically acceptable salt, prodrug or stereoisomer thereof.

In one embodiment, s is an integer from 2 to 12. In one embodiment, s is an integer from 2 to 8. In one embodiment, s is an integer from 2 to 6. In one embodiment, s is an integer from 2 to 4. In one embodiment, s is 2. In one embodiment, s is 3. In one embodiment, s is 4. In one embodiment, s is 5. In one embodiment, s is 6.

In one embodiment, G¹ is a bond. In one embodiment, G¹ is C₂-C₁₂ alkylene. In one embodiment, G¹ is C₄-C₈ alkylene. In one embodiment, G¹ is C₅-C₇ alkylene. In one embodiment, G¹ is C₂ alkylene. In one embodiment, G¹ is C₃ alkylene. In one embodiment, G¹ is C₄ alkylene. In one embodiment, G¹ is C₅ alkylene. In one embodiment, G¹ is C₆ alkylene. In one embodiment, G¹ is C₇ alkylene. In one embodiment, G¹ is C₂-C₁₂ alkenylene. In one embodiment, G¹ is C₄-C₈ alkenylene. In one embodiment, G¹ is C₅-C₇ alkenylene. In one embodiment, G¹ is C₅ alkenylene. In one embodiment, G¹ is C₇ alkenylene. In one embodiment, G is straight. In one embodiment, G¹ is branched. In one embodiment, G¹ is divalent. In one embodiment, G¹ is trivalent.

In one embodiment, G² is a bond. In one embodiment, G² is C₂-C₁₂ alkylene. In one embodiment, G² is C₄-C₈ alkylene. In one embodiment, G² is C₅-C₇ alkylene. In one embodiment, G² is C₂ alkylene. In one embodiment, G² is C₃ alkylene. In one embodiment, G² is C₄ alkylene. In one embodiment, G² is C₅ alkylene. In one embodiment, G² is C₆ alkylene. In one embodiment, G² is C₇ alkylene. In one embodiment, G² is C₂-C₁₂ alkenylene. In one embodiment, G² is C₄-C₈ alkenylene. In one embodiment, G² is C₅-C₇ alkenylene. In one embodiment, G² is C₅ alkenylene. In one embodiment, G² is C₇ alkenylene. In one embodiment, G² is straight. In one embodiment, G² is branched. In one embodiment, G² is divalent. In one embodiment, G² is trivalent.

In one embodiment, G¹ and G² are each independently C₂-C₁₂ alkylene. In one embodiment, G¹ and G² are each independently C₅ alkylene. In one embodiment, G¹ and G² are each independently C₇ alkylene.

In one embodiment, one or more —CH₂— in G¹ is replaced by —O—. In one embodiment, one or more non-terminal —CH₂— in G¹ is replaced by —O—. In one embodiment, one non-terminal —CH₂— in G¹ is replaced by —O—. In one embodiment, G¹ is (C₂-C₅ alkylene)-O—(C₂-C₆ alkylene).

In one embodiment, G¹ is

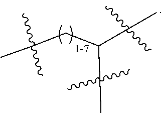

In one embodiment, G¹ is

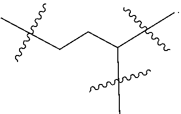 [SMILES: *CCC(*)*]

In one embodiment, G¹ is

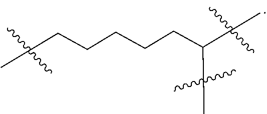 [SMILES: *CCCCCC(*)*]

In one embodiment, G¹ is

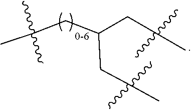

In one embodiment, G¹ is

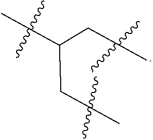 [SMILES: *CC(*)C*]

In one embodiment, G¹ is

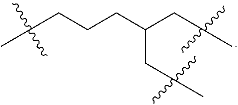 [SMILES: *CCCC(C*)C*]

In one embodiment, G¹ is

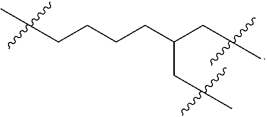 [SMILES: *CCCCC(C*)C*]

In one embodiment, G¹ is

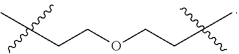 [SMILES: *CCOCC*]

In one embodiment, G¹ is

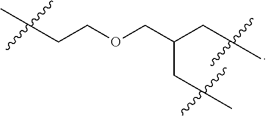 [SMILES: *CCOCC(C*)C*]

In one embodiment, one or more —CH₂— in G² is replaced by —O—. In one embodiment, one or more non-terminal —CH₂— in G² is replaced by —O—. In one embodiment, one non-terminal —CH₂— in G² is replaced by —O—. In one embodiment, G² is (C₂-C₅ alkylene)-O—(C₂-C₆ alkylene).

In one embodiment, G² is

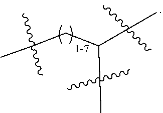

In one embodiment, G² is

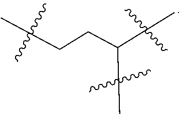 [SMILES: *CCC(*)*]

In one embodiment, G² is

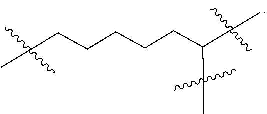 [SMILES: *CCCCCC(*)*]

In one embodiment, G² is

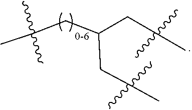

In one embodiment, G² is

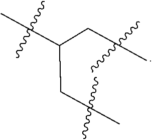 [SMILES: *CC(*)C*]

In one embodiment, G² is

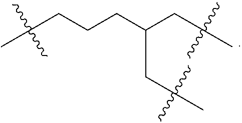 [SMILES: *CCCC(C*)C*]

In one embodiment, G² is

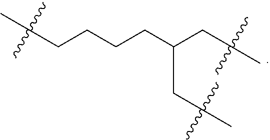 [SMILES: *CCCCC(C*)C*]

In one embodiment, G² is

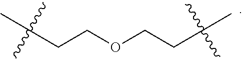 [SMILES: *CCOCC*]

In one embodiment, G² is

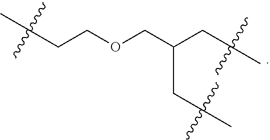 [SMILES: *CCOCC(C*)C*]

In one embodiment, the compound is a compound of Formula (IV):

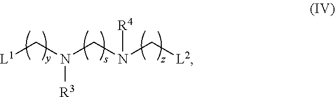

wherein s is an integer from 2 to 12,
y is an integer from 2 to 12; and
z is an integer from 2 to 12;
   or a pharmaceutically acceptable salt, prodrug or stereoisomer thereof.

In one embodiment, the compound is a compound of Formula (IV-A), (IV-B), (IV-C), (IV-D), (IV-E), (IV-F), (IV-G), or (IV-H):

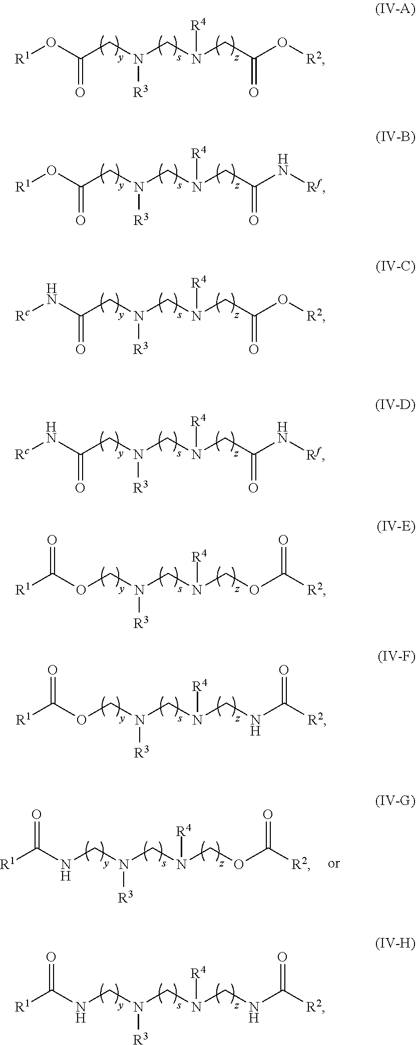

or a pharmaceutically acceptable salt, prodrug or stereoisomer thereof.

In one embodiment, the compound is a compound of Formula (V):

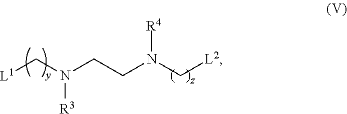

wherein y is an integer from 2 to 12; and
z is an integer from 2 to 12;
   or a pharmaceutically acceptable salt, prodrug or stereoisomer thereof.

In one embodiment, the compound is a compound of Formula (V-A), (V-B), (V-C), (V-D), (V-E), (V-F), (V-G), or (V-H):

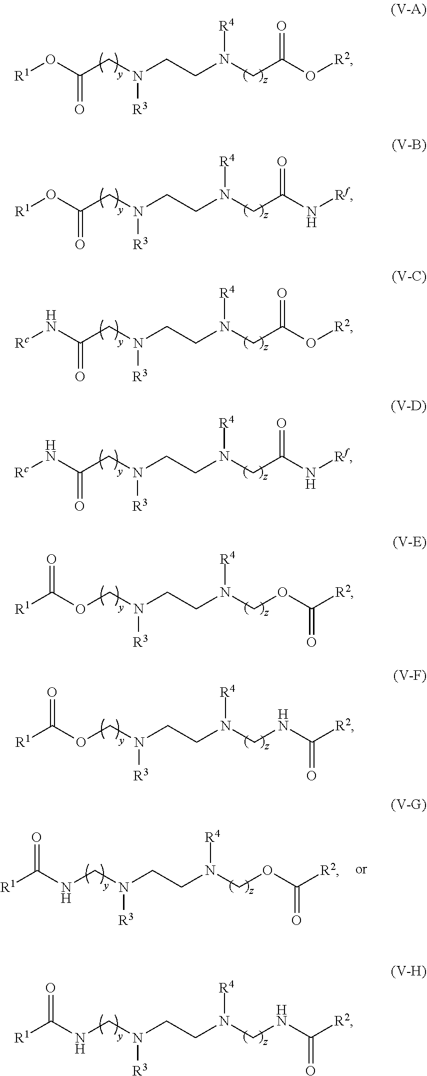

or a pharmaceutically acceptable salt, prodrug or stereoisomer thereof.

In one embodiment, y and z are each independently an integer from 2 to 10. In one embodiment, y and z are each independently an integer from 2 to 6. In one embodiment, y and z are each independently an integer from 4 to 10.

In one embodiment, y and z are different. In one embodiment, y and z are the same. In one embodiment, y and z are the same and are selected from 4, 5, 6, 7, 8, and 9. In one embodiment, y is 5 and z is 5.

In one embodiment, s is an integer from 2 to 12. In one embodiment, s is an integer from 2 to 8. In one embodiment, s is an integer from 2 to 6. In one embodiment, s is an integer from 2 to 4. In one embodiment, s is 2. In one embodiment, s is 3. In one embodiment, s is 4. In one embodiment, s is 5. In one embodiment, s is 6.

In one embodiment, y is 5, z is 5, and s is 2.

In one embodiment, L¹ is R¹.

In one embodiment, L¹ is —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c}), or -(4- to 8-membered heterocyclylene)-R¹. In one embodiment, L¹ is —OC(═O)R¹, —C(═O)OR¹, —C(═O)SR¹, —SC(═O)R¹, —NR^{a}C(═O)R¹, or —C(═O)NR^{b}R^{c}. In one embodiment, L¹ is —OC(═O)R¹, —C(═O)OR¹, —NR^{a}C(═O)R¹, or —C(═O)NR^{b}R^{c}. In one embodiment, L¹ is —OC(═O)R¹. In one embodiment, L¹ is —C(═O)OR¹. In one embodiment, L¹ is —NR^{a}C(═O)R¹. In one embodiment, L¹ is —C(═O)NR^{b}R^{c}. In one embodiment, L¹ is —OR¹. In one embodiment, L¹ is —NR^{a}P(═O)(OR^{b})(OR^{c}). In one embodiment, L¹ is -(4- to 8-membered heterocyclylene)-R¹. In one embodiment, L¹ is

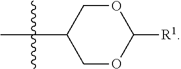

In one embodiment, L² is R².

In one embodiment, L² is —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), or —NR^{d}P(═O)(OR^{e})(OR^{f}), or -(4- to 8-membered heterocyclylene)-R². In one embodiment, L² is —OC(═O)R², —C(═O)OR², —C(═O)SR², —SC(═O)R², —NR^{d}C(═O)R², or —C(═O)NR^{e}R^{f}. In one embodiment, L² is —OC(═O)R², —C(═O)OR², —NR^{d}C(═O)R², or —C(═O)NR^{e}R^{f}. In one embodiment, L² is —OC(═O)R². In one embodiment, L² is —C(═O)OR². In one embodiment, L² is —NR^{d}C(═O)R². In one embodiment, L² is —C(═O)NR^{e}R^{f}. In one embodiment, L² is —OR². In one embodiment, L² is —NR^{d}P(═O)(OR^{e})(OR^{f}). In one embodiment, L² is -(4- to 8-membered heterocyclylene)-R². In one embodiment, L² is

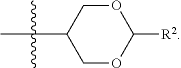

In one embodiment, L¹ is —C(═O)OR¹ or —C(═O)NR^{b}R^{c}; and L² is —C(═O)OR² or —C(═O)NR^{e}R^{f}. In one embodiment, L¹ is —C(═O)OR¹ and L² is —C(═O)OR². In one embodiment, L¹ is —C(═O)OR¹ and L² is —C(═O)NR^{e}R^{f}. In one embodiment, L¹ is —C(═O)NR^{b}R^{c} and L² is —C(═O)OR². In one embodiment, L¹ is —C(═O)NR^{b}R^{c} and L² is —C(═O)NR^{e}R^{f}.

In one embodiment, L¹ is —OC(═O)R¹ or —NR^{a}C(═O)R¹; and L² is —OC(═O)R² or —NR^{d}C(═O)R². In one embodiment, L¹ is —OC(═O)R¹ and L² is —OC(═O)R². In one embodiment, L¹ is —OC(═O)R¹ and L² is —NR^{d}C(═O)R². In one embodiment, L¹ is —NR^{a}C(═O)R¹ and L² is —OC(═O)R². In one embodiment, L¹ is —NR^{a}C(═O)R¹ and L² is —NR^{d}C(═O)R².

In one embodiment, L¹ is —OR¹ and L² is —C(═O)OR². In one embodiment, L¹ is —OR¹ and L² is —C(═O)NR^{e}R^{f}. In one embodiment, L¹ is —C(═O)OR¹ and L² is —OR². In one embodiment, L¹ is —C(═O)NR^{b}R^{c} and L² is —OR².

In one embodiment, the compound is a compound of Formula (VI):

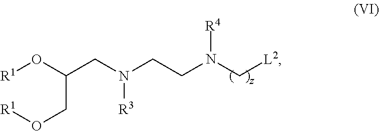

wherein z is an integer from 2 to 12;
   or a pharmaceutically acceptable salt, prodrug or stereoisomer thereof.

In one embodiment, z is an integer from 2 to 10. In one embodiment, z is an integer from 2 to 6. In one embodiment, z is an integer from 4 to 10. In one embodiment, z is selected from 4, 5, 6, 7, 8, and 9. In one embodiment, z is 5.

In one embodiment, R³ is C₁-C₁₂ alkyl. In one embodiment, R³ is C₁-C₅ alkyl. In one embodiment, R³ is C₁-C₆ alkyl. In one embodiment, R³ is C₁-C₄ alkyl. In one embodiment, the alkyl is straight alkyl. In one embodiment, the alkyl is branched alkyl. In one embodiment, R³ is methyl. In one embodiment, R³ is ethyl. In one embodiment, R³ is n-propyl. In one embodiment, R³ is isopropyl. In one embodiment, R³ is n-butyl. In one embodiment, R³ is n-pentyl. In one embodiment, R³ is n-hexyl. In one embodiment, R³ is n-octyl. In one embodiment, R³ is n-nonyl.

In one embodiment, R³ is C₂-C₁₂ alkenyl. In one embodiment, R³ is C₂-C₈ alkenyl. In one embodiment, R³ is C₂-C₄ alkenyl. In one embodiment, the alkenyl is straight alkenyl. In one embodiment, the alkenyl is branched alkenyl. In one embodiment, R³ is ethenyl. In one embodiment, R³ is allyl.

In one embodiment, R³ is C₂-C₁₂ alkynyl. In one embodiment, R³ is C₂-C₈ alkynyl. In one embodiment, R³ is C₂-C₄ alkynyl. In one embodiment, the alkynyl is straight alkynyl. In one embodiment, the alkynyl is branched alkynyl.

In one embodiment, R³ is C₃-C₈ cycloalkyl. In one embodiment, R³ is cyclopropyl. In one embodiment, R³ is cyclobutyl. In one embodiment, R³ is cyclopentyl. In one embodiment, R³ is cyclohexyl. In one embodiment, R³ is cycloheptyl. In one embodiment, R³ is cyclooctyl.

In one embodiment, R³ is C₃-C₈ cycloalkenyl. In one embodiment, R³ is cyclopropenyl. In one embodiment, R³ is cyclobutenyl. In one embodiment, R³ is cyclopentenyl. In one embodiment, R³ is cyclohexenyl. In one embodiment, R³ is cycloheptenyl. In one embodiment, R³ is cyclooctenyl.

In one embodiment, the R³ is 4- to 8-membered heterocyclyl. In one embodiment, the R³ is 4- to 8-membered heterocycloalkyl. In one embodiment, the R³ is oxetanyl. In one embodiment, the R³ is tetrahydrofuranyl. In one embodiment, the R³ is tetrahydropyranyl. In one embodiment, the R³ is tetrahydrothiopyranyl.

In one embodiment, R³ is C₆-C₁₀ aryl. In one embodiment, R³ is phenyl.

In one embodiment, R³ is 5- to 10-membered heteroaryl. In one embodiment, R³ is 5-membered heteroaryl. In one embodiment, R³ is 6-membered heteroaryl.

In one embodiment, R³, G¹ or part of G¹, together with the nitrogen to which they are attached form a cyclic moiety.

In one embodiment, the compound is a compound of Formula (VII):

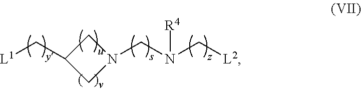

wherein s is an integer from 2 to 12,
u is 1, 2, or 3;
v is 1, 2, or 3;
y′ is an integer from 0 to 10; and
z is an integer from 2 to 12;
   or a pharmaceutically acceptable salt, prodrug or stereoisomer thereof.

In one embodiment, R³, G³ or part of G³, together with the nitrogen to which they are attached form a cyclic moiety.

In one embodiment, the compound is a compound of Formula (VIII-A), (VIII-B), (VIII-C), (VIII-D), (VIII-E), (VIII-F), or (VIII-G):

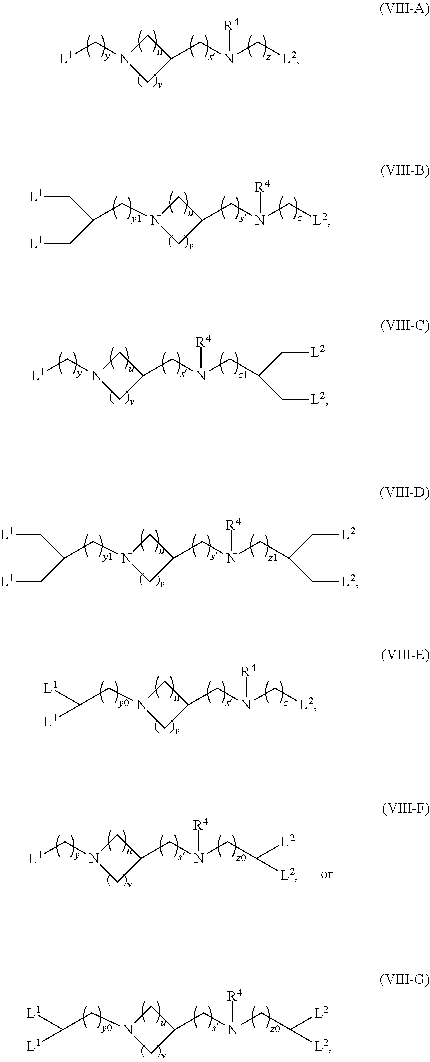

wherein s′ is an integer from 0 to 10,
u is 1, 2, or 3;
v is 1, 2, or 3;
y is an integer from 2 to 12;
z is an integer from 2 to 12;
y0 is an integer from 1 to 11;
z0 is an integer from 1 to 11;
y1 is an integer from 0 to 9; and
z1 is an integer from 0 to 9;
   or a pharmaceutically acceptable salt, prodrug or stereoisomer thereof.

In one embodiment, u is 1. In one embodiment, u is 2. In one embodiment, u is 3. In one embodiment, v is 1. In one embodiment, v is 2. In one embodiment, v is 3. In one embodiment, u is 1 and v is 1. In one embodiment, u is 2 and v is 2. In one embodiment, u is 3 and v is 3.

In one embodiment, the compound is a compound of Formula (IX-A), (IX-B), (IX-C), (IX-D), (IX-E), (IX-F), (IX-G), (IX-H), (IX-J), (IX-J), (IX-K), (IX-L), (IX-M), (IX-N), (IX-O), (IX-P), (IX-Q), (IX-R), (IX-S), (IX-T), (IX-U), (IX-V), (IX-W), (IX-X), (IX-Y), (IX-Z), or (IX-AA):

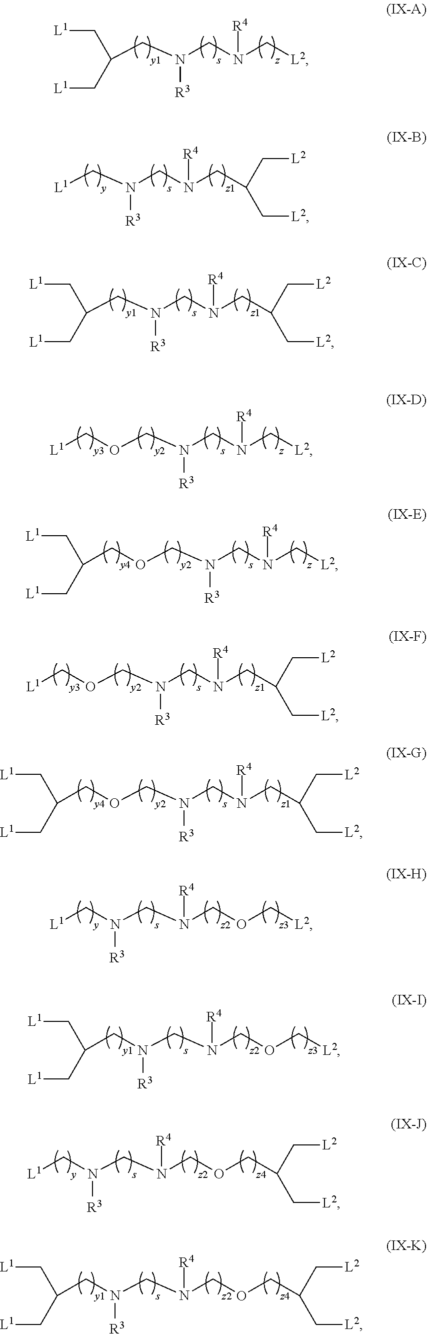

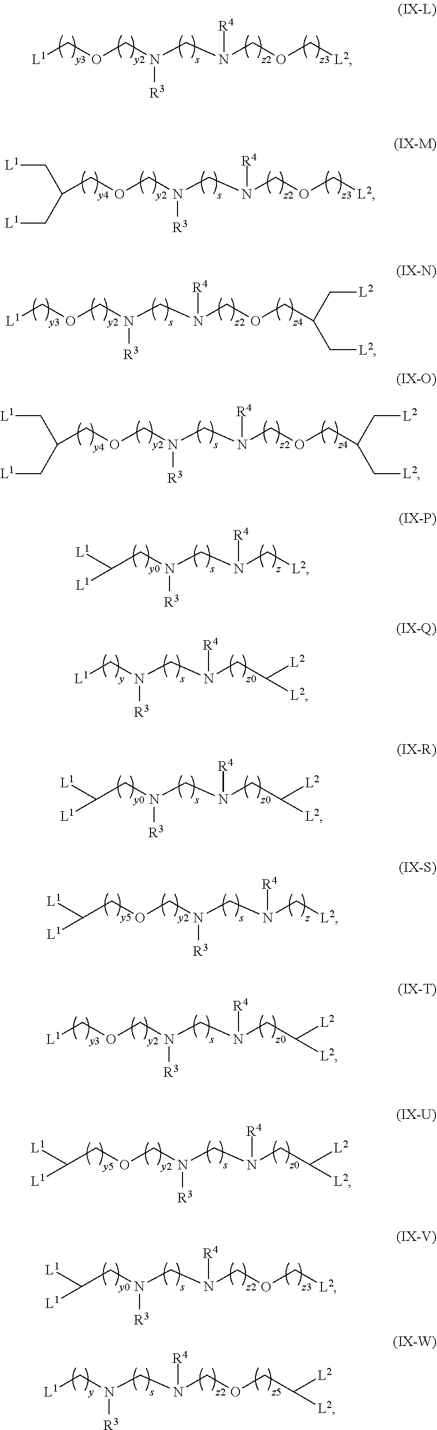

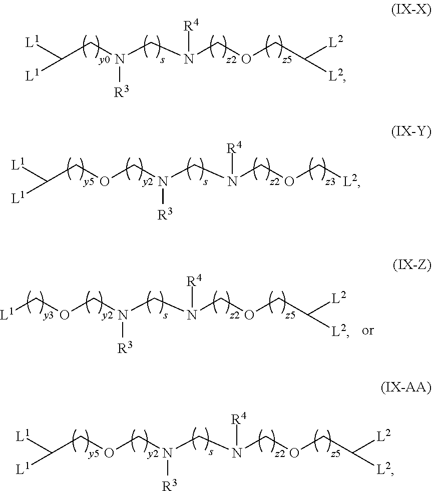

wherein s is an integer from 2 to 12,
y is an integer from 2 to 12;
z is an integer from 2 to 12;
y0 is an integer from 1 to 11;
z0 is an integer from 1 to 11;
y1 is an integer from 0 to 9;
z1 is an integer from 0 to 9;
y2 is an integer from 2 to 5;
y3 is an integer from 2 to 6;
y4 is an integer from 0 to 3;
y5 is an integer from 1 to 5;
z2 is an integer from 2 to 5;
z3 is an integer from 2 to 6;
z4 is an integer from 0 to 3; and
z5 is an integer from 1 to 5;
   or a pharmaceutically acceptable salt, prodrug or stereoisomer thereof.

In one embodiment, y0 is an integer from 1 to 7. In one embodiment, y0 is 1. In one embodiment, y0 is 2. In one embodiment, y0 is 3. In one embodiment, y0 is 4. In one embodiment, y0 is 5. In one embodiment, y0 is 6. In one embodiment, y0 is 7. In one embodiment, z0 is an integer from 1 to 7. In one embodiment, z0 is 1. In one embodiment, z0 is 2. In one embodiment, z0 is 3. In one embodiment, z0 is 4. In one embodiment, z0 is 5. In one embodiment, z0 is 6. In one embodiment, z0 is 7.

In one embodiment, y1 is an integer from 2 to 6. In one embodiment, y1 is 2. In one embodiment, y1 is 3. In one embodiment, y1 is 4. In one embodiment, y1 is 5. In one embodiment, y1 is 6. In one embodiment, z1 is an integer from 2 to 6. In one embodiment, z1 is 2. In one embodiment, z1 is 3. In one embodiment, z1 is 4. In one embodiment, z1 is 5. In one embodiment, z1 is 6.

In one embodiment, y2 is 2. In one embodiment, y2 is 3. In one embodiment, y2 is 4. In one embodiment, y2 is 5. In one embodiment, z2 is 2. In one embodiment, z2 is 3. In one embodiment, z2 is 4. In one embodiment, z2 is 5.

In one embodiment, y3 is 2. In one embodiment, y3 is 3. In one embodiment, y3 is 4. In one embodiment, y3 is 5. In one embodiment, y3 is 6. In one embodiment, z3 is 2. In one embodiment, z3 is 3. In one embodiment, z3 is 4. In one embodiment, z3 is 5. In one embodiment, z3 is 6.

In one embodiment, y4 is 0. In one embodiment, y4 is 1. In one embodiment, y4 is 2. In one embodiment, y4 is 3. In one embodiment, z4 is 0. In one embodiment, z4 is 1. In one embodiment, z4 is 2. In one embodiment, z4 is 3.

In one embodiment, y5 is 1. In one embodiment, y5 is 2. In one embodiment, y5 is 3. In one embodiment, y5 is 4. In one embodiment, y5 is 5. In one embodiment, z5 is 1. In one embodiment, z5 is 2. In one embodiment, z5 is 3. In one embodiment, z5 is 4. In one embodiment, z5 is 5.

In one embodiment, y2 is 2 and y3 is 2. In one embodiment, y2 is 2 and y4 is 1. In one embodiment, z2 is 2 and z3 is 2. In one embodiment, z2 is 2 and z4 is 1.

In one embodiment, s, y, z, L¹ and L² are as defined elsewhere. In one embodiment, L¹ is —OR¹, —OC(═O)R¹, —C(═O)OR¹, or —C(═O)NR^{b}R^{c}; and L² is —OR², —OC(═O)R², —C(═O)OR², or —C(═O)NR^{e}R^{f}. In one embodiment, where there are two L¹, each L¹ is independently —OC(═O)R¹. In one embodiment, where there are two L², each L² is independently —OC(═O)R². In one embodiment, where there is only one L¹, L¹ is —C(═O)OR¹. In one embodiment, where there is only one L¹, L¹ is —C(═O)NR^{b}R^{c}. In one embodiment, where there is only one L², L² is —C(═O)OR². In one embodiment, where there is only one L², L² is —C(═O)NR^{e}R^{f}.

In one particularly embodiment of any one of Formulas (IX-A) to (IX-AA), when there is only one L¹, L¹ is —C(═O)OR¹. In another embodiment, L¹ is —C(═O)NR^{b}R^{c}. In one embodiment, R¹ or R^{c} is —R⁷—CH(R⁸)(R⁹), wherein R⁷ is C₀-C₁ alkylene, and R⁸ and R⁹ are independently C₄-C₈ alkyl.

In one particularly embodiment of any one of Formulas (IX-A) to (IX-AA), when there is only one L², L² is —C(═O)OR². In another embodiment, L² is —C(═O)NR^{e}R^{f}. In one embodiment, R² or R^{f} is —R⁷—CH(R⁸)(R⁹), wherein R⁷ is C₀-C₁ alkylene, and R⁸ and R⁹ are independently C₄-C₈ alkyl.

In one particularly embodiment of any one of Formulas (IX-A) to (IX-AA), when there is a

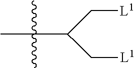

moiety, each L¹ is independently —OC(═O)R¹. In one embodiment, each R¹ is independently straight C₇-C₁₁ alkyl.

In one particularly embodiment of any one of Formulas (IX-A) to (IX-AA), when L² there is a

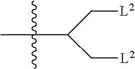

moiety, each L² is independently —OC(═O)R². In one embodiment, each R¹ is independently straight C₇-C₁₁ alkyl.

In one particularly embodiment of any one of Formulas (IX-A) to (IX-AA), when there is a

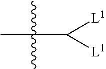

moiety, each L¹ is independently —OR¹. In another embodiment, each L¹ is independently —C(═O)OR¹. In one embodiment, each R¹ is independently straight C₇-C₁₁ alkyl.

In one particularly embodiment of any one of Formulas (IX-A) to (IX-AA), when there is a

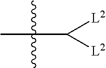

moiety, each L² is independently —OR². In another embodiment, each L² is independently —C(═O)OR². In one embodiment, each R² is independently straight C₇-C₁₁ alkyl.

In one embodiment, R³ is unsubstituted.

In one embodiment, R³ is substituted with one or more substituents selected from the group consisting of C₁-C₆ alkyl, halo, C₁-C₆ haloalkyl, nitro, oxo, —OR^{g}, —NR^{g}C(═O)R^{h}, —C(═O)NR^{g}R^{h}, —C(═O)R^{h}, —OC(═O)R^{h}, —C(═O)OR^{h} and —O—Rⁱ—OH, wherein:
R⁹ is at each occurrence independently H or C₁-C₆ alkyl;
R^{h} is at each occurrence independently C₁-C₆ alkyl; and
Rⁱ is at each occurrence independently C₁-C₆ alkylene.

In one embodiment, R³ is substituted with one or more C₁-C₆ alkyl (e.g., methyl). In one embodiment, R³ is substituted with one or more halo (e.g., —F). In one embodiment, R³ is substituted with one or more C₁-C₆ haloalkyl (e.g., —CF₃). In one embodiment, R³ is substituted with one or more hydroxyl. In one embodiment, R³ is substituted with one hydroxyl.

In one embodiment, R³ is substituted with one or more C₃-C₈ cycloalkyl, C₆-C₁₀ aryl, or 5- to 10-membered heteroaryl, each of which is optionally substituted. In one embodiment, R³ is C₁-C₆ alkyl (e.g., methyl) substituted with one or more C₃-C₈ cycloalkyl, C₆-C₁₀ aryl, or 5- to 10-membered heteroaryl, each of which is optionally substituted. In one embodiment, the C₃-C₈ cycloalkyl, C₆-C₁₀ aryl, or 5- to 10-membered heteroaryl is unsubstituted. In one embodiment, the C₃-C₈ cycloalkyl, C₆-C₁₀ aryl, or 5- to 10-membered heteroaryl is substituted with one or more C₁-C₆ alkyl, halo, C₁-C₆ haloalkyl, nitro, hydroxyl, or cyano.

In one embodiment, R⁴ is C₁-C₁₂ alkyl. In one embodiment, R⁴ is C₁-C₈ alkyl. In one embodiment, R⁴ is C₁-C₆ alkyl. In one embodiment, R⁴ is C₁-C₄ alkyl. In one embodiment, R⁴ is methyl. In one embodiment, R⁴ is ethyl. In one embodiment, R⁴ is n-propyl. In one embodiment, R⁴ is isopropyl. In one embodiment, R⁴ is n-butyl. In one embodiment, R⁴ is n-pentyl. In one embodiment, R⁴ is n-hexyl. In one embodiment, R⁴ is n-octyl. In one embodiment, R⁴ is n-nonyl.

In one embodiment, R⁴ is C₃-C₈ cycloalkyl. In one embodiment, R⁴ is cyclopropyl. In one embodiment, R⁴ is cyclobutyl. In one embodiment, R⁴ is cyclopentyl. In one embodiment, R⁴ is cyclohexyl. In one embodiment, R⁴ is cycloheptyl. In one embodiment, R⁴ is cyclooctyl.

In one embodiment, R⁴ is unsubstituted.

In one embodiment, R⁴ is substituted with one or more substituents selected from the group consisting of oxo, —OR^{g}, —NR^{g}C(═O)R^{h}, —C(═O)NR^{g}R^{h}, —C(═O)R^{h}, OC(O)R^{h}, —C(═O)OR^{h}, —O—Rⁱ—OH, and —N(R¹⁰)R¹¹, wherein:
R^{g} is at each occurrence independently H or C₁-C₆ alkyl;
R^{h} is at each occurrence independently C₁-C₆ alkyl;
Rⁱ is at each occurrence independently C₁-C₆ alkylene;
R¹⁰ is hydrogen or C₁-C₆ alkyl;
R¹¹ is C₁-C₆ alkyl, C₃-C₈ cycloalkyl, or C₃-C₈ cycloalkenyl;
or R¹⁰ and R¹¹ together with the nitrogen to which they are attached form a cyclic moiety;
and R¹¹ or the cyclic moiety is optionally substituted with one or more of hydroxyl, oxo, —NH₂, —NH(C₁-C₆ alkyl), or —N(C₁-C₆ alkyl)₂.

In one embodiment, R⁴ is substituted with one or more hydroxyl. In one embodiment, R⁴ is substituted with one hydroxyl.

In one embodiment, R⁴ is a substituted C₁-C₁₂ alkyl. In one embodiment, R⁴ is —(CH₂)ₚQ, —(CH₂)ₚCHQR, —CHQR, or —CQ(R)₂, wherein Q is C₃-C₈ cycloalkyl, C₃-C₈ cycloalkenyl, C₃-C₈ cycloalkynyl, 4- to 8-membered heterocyclyl, C₆-C₁₀ aryl, 5- to 10-membered heteroaryl, —OR, —O(CH₂)ₚN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R²², —O(CH₂)ₚOR, —N(R)C(═NR²³)N(R)₂, —N(R)C(═CHR²³)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR²³)N(R)₂, —N(OR)C(═CHR²³)N(R)₂, —C(═NR²³)N(R)₂, —C(═NR²³)R, —C(O)N(R)OR, or —C(R)N(R)₂C(O)OR, and each p is independently 1, 2, 3, 4, or 5;
R²² is C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl;
R²³ is H, —CN, —NO₂, C₁-C₆ alkyl, —OR, —S(O)₂R, —S(O)₂N(R)₂, C₂-C₆ alkenyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl;
each R is independently H, C₁-C₃ alkyl, or C₂-C₃ alkenyl; or two R in a N(R)₂ moiety together with the nitrogen to which they are attached form a cyclic moiety; and
each X is independently F, Cl, Br, or I.

In one embodiment, R⁴ is —CH₂CH₂OH. In one embodiment, R⁴ is —CH₂CH₂CH₂OH. In one embodiment, R⁴ is —CH₂CH₂CH₂CH₂OH. In one embodiment, R⁴ is —CH₂CH₂OCH₂CH₂OH.

In one embodiment, R⁴ is substituted with one or more —N(R¹⁰)R¹¹. In one embodiment, R⁴ is substituted with one —N(R¹⁰)R¹¹.

In one embodiment, R¹⁰ is hydrogen.

In one embodiment, R¹¹ is C₃-C₈ cycloalkenyl. In one embodiment, R¹¹ is cyclobutenyl. In one embodiment, R¹¹ is substituted with one or more of oxo, —NH₂, —NH(C₁-C₆ alkyl), or —N(C₁-C₆ alkyl)₂.

In one embodiment, R¹⁰ and R¹¹ together with the nitrogen to which they are attached form a cyclic moiety. In one embodiment, the cyclic moiety is a 5- to 10-membered heteroaryl. In one embodiment, the cyclic moiety is pyrimidin-1-yl. In one embodiment, the cyclic moiety is purin-9-yl. In one embodiment, the cyclic moiety is substituted with one or more of oxo, —NH₂, —NH(C₁-C₆ alkyl), or —N(C₁-C₆ alkyl)₂.

In one embodiment, R⁴ is substituted with

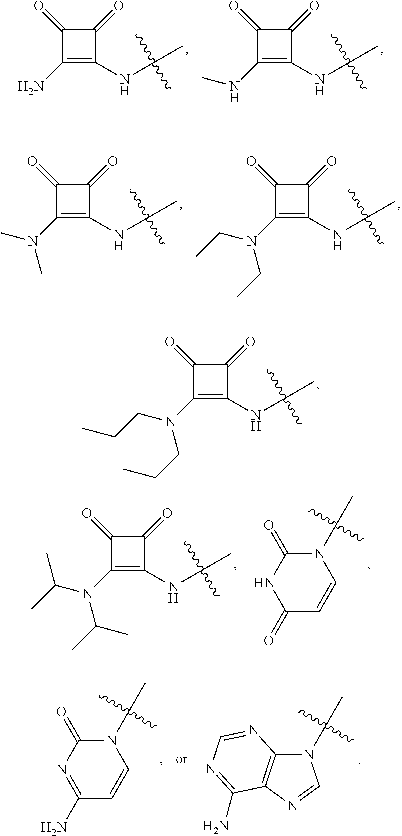

In one embodiment, R⁴ is substituted with

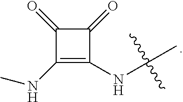 [SMILES: *Nc1c(NC)c(=O)c1=O]

In one embodiment, R⁴ is substituted with

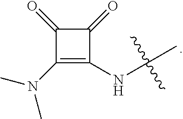 [SMILES: *Nc1c(N(C)C)c(=O)c1=O]

In one embodiment, R¹ is straight C₁-C₂₄ alkyl. In one embodiment, R¹ is straight C₇-C₁₅ alkyl. In one embodiment, R¹ is straight C₇ alkyl. In one embodiment, R¹ is straight C₈ alkyl. In one embodiment, R¹ is straight C₉ alkyl. In one embodiment, R¹ is straight C₁₀ alkyl. In one embodiment, R¹ is straight C₇ alkyl. In one embodiment, R¹ is straight C₁₂ alkyl. In one embodiment, R¹ is straight C₁₃ alkyl. In one embodiment, R¹ is straight C₁₄ alkyl. In one embodiment, R¹ is straight C₁₅ alkyl.

In one embodiment, R¹ is straight C₆-C₂₄ alkenyl. In one embodiment, R¹ is straight C₇-C₁₇ alkenyl. In one embodiment, R¹ is straight C₇ alkenyl. In one embodiment, R¹ is straight C₈ alkenyl. In one embodiment, R¹ is straight C₉ alkenyl. In one embodiment, R¹ is straight C₁₀ alkenyl. In one embodiment, R¹ is straight C₁₁ alkenyl. In one embodiment, R¹ is straight C₁₂ alkenyl. In one embodiment, R¹ is straight C₁₃ alkenyl. In one embodiment, R¹ is straight C₁₄ alkenyl. In one embodiment, R¹ is straight C₁₅ alkenyl. In one embodiment, R¹ is straight C₁₆ alkenyl. In one embodiment, R¹ is straight C₁₇ alkenyl.

In one embodiment, R¹ is branched C₆-C₂₄ alkyl. In one embodiment, R¹ is —R⁷—CH(R⁸)(R⁹), wherein R⁷ is C₀-C₅ alkylene, and R⁸ and R⁹ are independently C₂-C₁₀ alkyl. In one embodiment, R¹ is —R⁷—CH(R⁸)(R⁹), wherein R⁷ is C₀-C₁ alkylene, and R⁸ and R⁹ are independently C₄-C₈ alkyl.

In one embodiment, R¹ is branched C₆-C₂₄ alkenyl. In one embodiment, R¹ is —R⁷—CH(R⁸)(R⁹), wherein R⁷ is C₀-C₅ alkylene, and R⁸ and R⁹ are independently C₂-C₁₀ alkenyl. In one embodiment, R¹ is —R⁷—CH(R⁸)(R⁹), wherein R⁷ is C₀-C₁ alkylene, and R⁸ and R⁹ are independently C₆-C₁₀ alkenyl.

In one embodiment, R² is straight C₆-C₂₄ alkyl. In one embodiment, R² is straight C₇-C₁₅ alkyl. In one embodiment, R² is straight C₇ alkyl. In one embodiment, R² is straight C₈ alkyl. In one embodiment, R² is straight C₉ alkyl. In one embodiment, R² is straight C₁₀ alkyl. In one embodiment, R² is straight C₁₁ alkyl. In one embodiment, R² is straight C₁₂ alkyl. In one embodiment, R² is straight C₁₃ alkyl. In one embodiment, R² is straight C₁₄ alkyl. In one embodiment, R² is straight C₁₅ alkyl.

In one embodiment, R² is straight C₆-C₂₄ alkenyl. In one embodiment, R² is straight C₇-C₁₇ alkenyl. In one embodiment, R² is straight C₇ alkenyl. In one embodiment, R² is straight C₈ alkenyl. In one embodiment, R² is straight C₉ alkenyl. In one embodiment, R² is straight C₁₀ alkenyl. In one embodiment, R² is straight C₁₁ alkenyl. In one embodiment, R² is straight C₁₂ alkenyl. In one embodiment, R² is straight C₁₃ alkenyl. In one embodiment, R² is straight C₁₄ alkenyl. In one embodiment, R² is straight C₁₅ alkenyl. In one embodiment, R² is straight C₁₆ alkenyl. In one embodiment, R² is straight C₁₇ alkenyl.

In one embodiment, R² is branched C₆-C₂₄ alkyl. In one embodiment, R² is —R⁷—CH(R⁸)(R⁹), wherein R⁷ is C₀-C₅ alkylene, and R⁸ and R⁹ are independently C₂-C₁₀ alkyl. In one embodiment, R² is —R⁷—CH(R⁸)(R⁹), wherein R⁷ is C₀-C₁ alkylene, and R⁸ and R⁹ are independently C₄-C₈ alkyl.

In one embodiment, R² is branched C₆-C₂₄ alkenyl. In one embodiment, R² is —R⁷—CH(R⁸)(R⁹), wherein R⁷ is C₀-C₅ alkylene, and R⁸ and R⁹ are independently C₂-C₁₀ alkenyl. In one embodiment, R² is —R⁷—CH(R⁸)(R⁹), wherein R⁷ is C₀-C₁ alkylene, and R⁸ and R⁹ are independently C₆-C₁₀ alkenyl.

In one embodiment, R^{c} is straight C₆-C₂₄ alkyl. In one embodiment, R^{c} is straight C₇-C₁₅ alkyl. In one embodiment, R^{c} is straight C₇ alkyl. In one embodiment, R^{c} is straight C₈ alkyl. In one embodiment, R^{c} is straight C₉ alkyl. In one embodiment, R^{c} is straight C₁₀ alkyl. In one embodiment, R^{c} is straight C₁₁ alkyl. In one embodiment, R^{c} is straight C₁₂ alkyl. In one embodiment, R^{c} is straight C₁₃ alkyl. In one embodiment, R^{c} is straight C₁₄ alkyl. In one embodiment, R^{c} is straight C₁₅ alkyl.

In one embodiment, R^{c} is straight C₆-C₂₄ alkenyl. In one embodiment, R^{c} is straight C₇-C₁₇ alkenyl. In one embodiment, R^{c} is straight C₇ alkenyl. In one embodiment, R^{c} is straight C₈ alkenyl. In one embodiment, R^{c} is straight C₉ alkenyl. In one embodiment, R^{c} is straight C₁₀ alkenyl. In one embodiment, R^{c} is straight C₁₁ alkenyl. In one embodiment, R^{c} is straight C₁₂ alkenyl. In one embodiment, R^{c} is straight C₁₃ alkenyl. In one embodiment, R^{c} is straight C₁₄ alkenyl. In one embodiment, R^{c} is straight C₁₅ alkenyl. In one embodiment, R^{c} is straight C₁₆ alkenyl. In one embodiment, R^{c} is straight C₁₇ alkenyl.

In one embodiment, R^{c} is branched C₆-C₂₄ alkyl. In one embodiment, R^{c} is —R⁷—CH(R⁸)(R⁹), wherein R⁷ is C₀-C₅ alkylene, and R⁸ and R⁹ are independently C₂-C₁₀ alkyl. In one embodiment, R^{c} is —R⁷—CH(R⁸)(R⁹), wherein R⁷ is C₀-C₁ alkylene, and R⁸ and R⁹ are independently C₄-C₈ alkyl.

In one embodiment, R^{c} is branched C₆-C₂₄ alkenyl. In one embodiment, R^{c} is —R⁷—CH(R⁸)(R⁹), wherein R⁷ is C₀-C₅ alkylene, and R⁸ and R⁹ are independently C₂-C₁₀ alkenyl. In one embodiment, R^{c} is —R⁷—CH(R⁸)(R⁹), wherein R⁷ is C₀-C₁ alkylene, and R⁸ and R⁹ are independently C₆-C₁₀ alkenyl.

In one embodiment, R^{f} is straight C₆-C₂₄ alkyl. In one embodiment, R^{f} is straight C₇-C₁₅ alkyl. In one embodiment, R^{f} is straight C₇ alkyl. In one embodiment, R^{f} is straight C₈ alkyl. In one embodiment, R^{f} is straight C₉ alkyl. In one embodiment, R^{f} is straight C₁₀ alkyl. In one embodiment, R^{f} is straight C₁₁ alkyl. In one embodiment, R^{f} is straight C₁₂ alkyl. In one embodiment, R^{f} is straight C₁₃ alkyl. In one embodiment, R^{f} is straight C₁₄ alkyl. In one embodiment, R^{f} is straight C₁₅ alkyl.

In one embodiment, R^{f} is straight C₆-C₂₄ alkenyl. In one embodiment, R^{f} is straight C₇-C₁₇ alkenyl. In one embodiment, R^{f} is straight C₇ alkenyl. In one embodiment, R^{f} is straight C₈ alkenyl. In one embodiment, R^{f} is straight C₉ alkenyl. In one embodiment, R^{f} is straight C₁₀ alkenyl. In one embodiment, R^{f} is straight C₁₁ alkenyl. In one embodiment, R^{f} is straight C₁₂ alkenyl. In one embodiment, R^{f} is straight C₁₃ alkenyl. In one embodiment, R^{f} is straight C₁₄ alkenyl. In one embodiment, R^{f} is straight C₁₅ alkenyl. In one embodiment, R^{f} is straight C₁₆ alkenyl. In one embodiment, R^{f} is straight C₁₇ alkenyl.

In one embodiment, R^{f} is branched C₆-C₂₄ alkyl. In one embodiment, R^{f} is —R⁷—CH(R⁸)(R⁹), wherein R⁷ is C₀-C₅ alkylene, and R⁸ and R⁹ are independently C₂-C₁₀ alkyl. In one embodiment, R^{f} is —R⁷—CH(R⁸)(R⁹), wherein R⁷ is C₀-C₁ alkylene, and R⁸ and R⁹ are independently C₄-C₈ alkyl.

In one embodiment, R^{f} is branched C₆-C₂₄ alkenyl. In one embodiment, R^{f} is —R⁷—CH(R⁸)(R⁹), wherein R⁷ is C₀-C₅ alkylene, and R⁸ and R⁹ are independently C₂-C₁₀ alkenyl. In one embodiment, R^{f} is —R⁷—CH(R⁸)(R⁹), wherein R⁷ is C₀-C₁ alkylene, and R⁸ and R⁹ are independently C₆-C₁₀ alkenyl.

In one embodiment, R¹, R², R^{c}, and R^{f} are each independently straight C₆-C₁₈ alkyl, straight C₆-C₁₈ alkenyl, or —R⁷—CH(R⁸)(R⁹), wherein R⁷ is C₀-C₅ alkylene, and R⁸ and R⁹ are independently C₂-C₁₀ alkyl or C₂-C₁₀ alkenyl.

In one embodiment, R¹, R², R^{c}, and R^{f} are each independently straight C₇-C₁₅ alkyl, straight C₇-C₁₅ alkenyl, or —R⁷—CH(R⁸)(R⁹), wherein R⁷ is C₀-C₁ alkylene, and R⁸ and R⁹ are independently C₄-C₈ alkyl or C₆-C₁₀ alkenyl.

In one embodiment, R¹, R², R^{c}, and R^{f} are each independently one of the following structures.

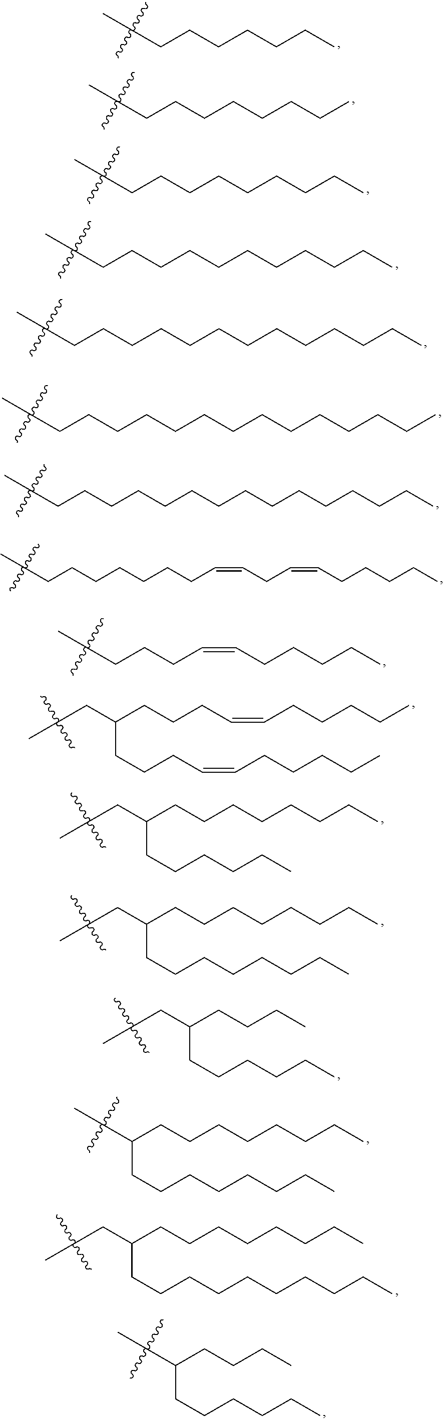

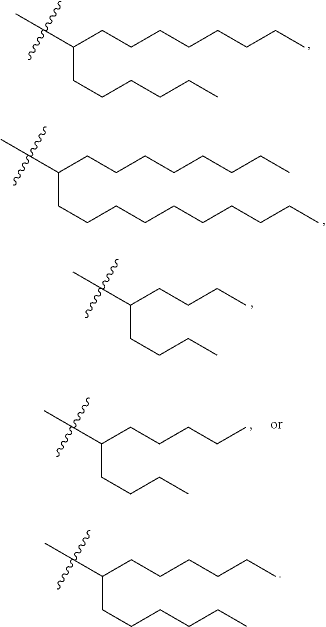

In one embodiment, R^{a} is H. In one embodiment, R^{d} is H. In one embodiment, R^{a}, R^{b}, R^{d}, and R^{e} are each independently H. In one embodiment, R^{b} is C₁-C₂₄ alkyl. In one embodiment, R^{b} is C₁-C₁₂ alkyl. In one embodiment, R^{b} is C₂-C₂₄ alkenyl. In one embodiment, R^{b} is C₂-C₁₂ alkenyl. In one embodiment, R^{e} is C₁-C₂₄ alkyl. In one embodiment, R^{e} is C₁-C₁₂ alkyl. In one embodiment, R^{e} is C₂-C₂₄ alkenyl. In one embodiment, R^{e} is C₂-C₁₂ alkenyl.

In one embodiment, the compound is a compound in Table 1, or a pharmaceutically acceptable salt, prodrug or stereoisomer thereof.

TABLE 1

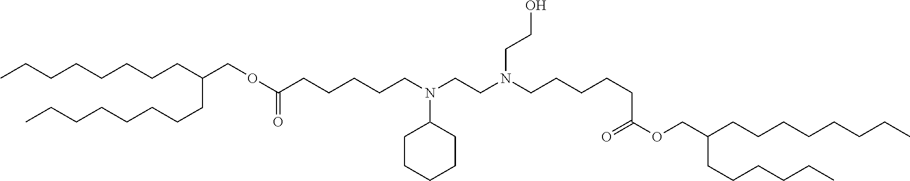 [SMILES: CCCCCCCCC(CCCCCC)COC(=O)CCCCCN(CCO)CCN(CCCCCC(=O)OCC(CCCCCCCC)CCCCCCCC)C1CCCCC1]

Compound 1

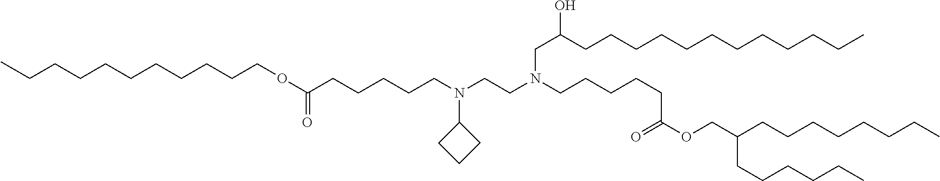 [SMILES: CCCCCCCCCCCCC(O)CN(CCCCCC(=O)OCC(CCCCCC)CCCCCCCC)CCN(CCCCCC(=O)OCCCCCCCCCCC)C1CCC1]

Compound 2

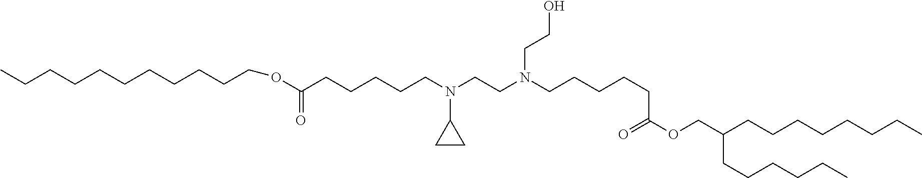 [SMILES: CCCCCCCCCCCOC(=O)CCCCCN(CCN(CCO)CCCCCC(=O)OCC(CCCCCC)CCCCCCCC)C1CC1]

Compound 3

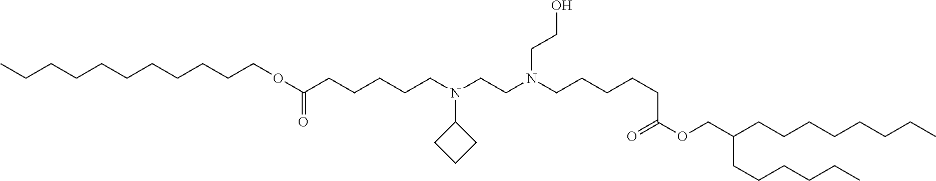 [SMILES: CCCCCCCCCCCOC(=O)CCCCCN(CCN(CCO)CCCCCC(=O)OCC(CCCCCC)CCCCCCCC)C1CCC1]

Compound 4

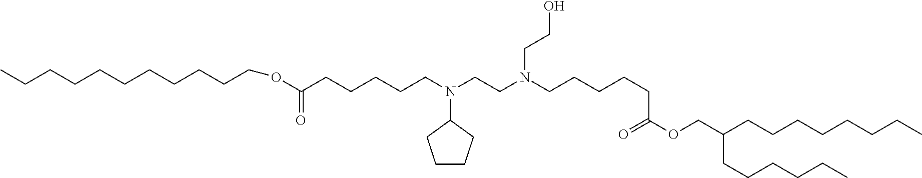 [SMILES: CCCCCCCCCCCOC(=O)CCCCCN(CCN(CCO)CCCCCC(=O)OCC(CCCCCC)CCCCCCCC)C1CCCC1]

Compound 5

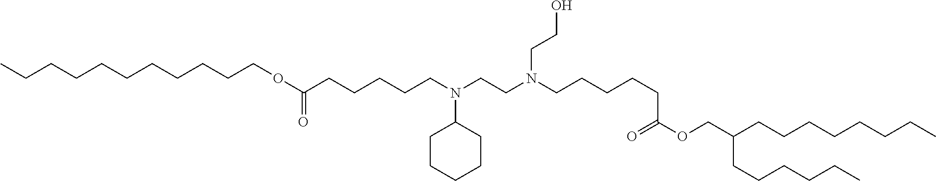 [SMILES: CCCCCCCCCCCOC(=O)CCCCCN(CCN(CCO)CCCCCC(=O)OCC(CCCCCC)CCCCCCCC)C1CCCCC1]

Compound 6

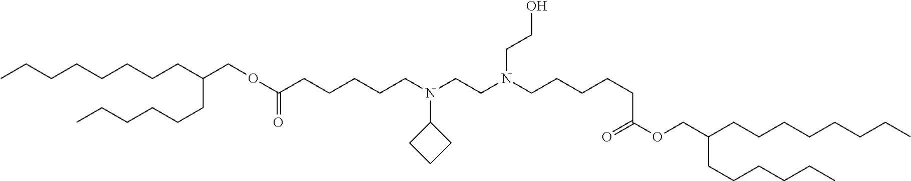 [SMILES: CCCCCCCCC(CCCCCC)COC(=O)CCCCCN(CCO)CCN(CCCCCC(=O)OCC(CCCCCC)CCCCCCCC)C1CCC1]

Compound 7

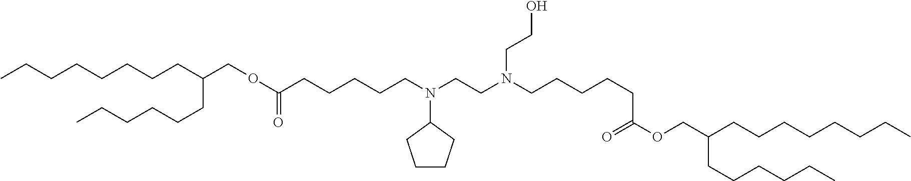 [SMILES: CCCCCCCCC(CCCCCC)COC(=O)CCCCCN(CCO)CCN(CCCCCC(=O)OCC(CCCCCC)CCCCCCCC)C1CCCC1]

Compound 8

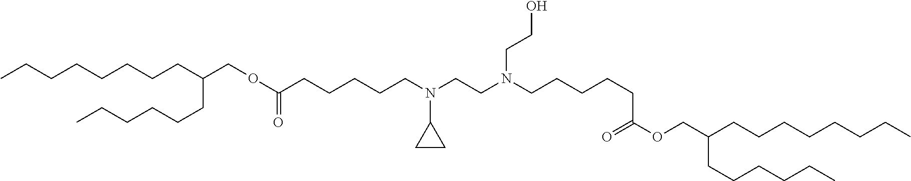 [SMILES: CCCCCCCCC(CCCCCC)COC(=O)CCCCCN(CCO)CCN(CCCCCC(=O)OCC(CCCCCC)CCCCCCCC)C1CC1]

Compound 9

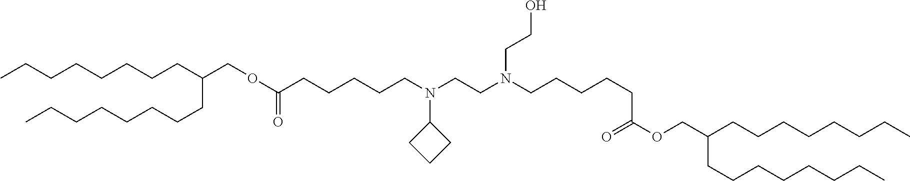 [SMILES: CCCCCCCCC(CCCCCCCC)COC(=O)CCCCCN(CCO)CCN(CCCCCC(=O)OCC(CCCCCCCC)CCCCCCCC)C1CCC1]

Compound 10

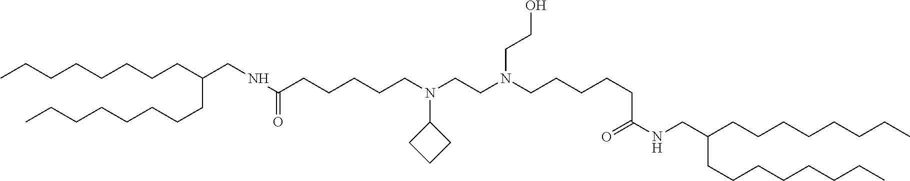 [SMILES: CCCCCCCCC(CCCCCCCC)CNC(=O)CCCCCN(CCO)CCN(CCCCCC(=O)NCC(CCCCCCCC)CCCCCCCC)C1CCC1]

Compound 11

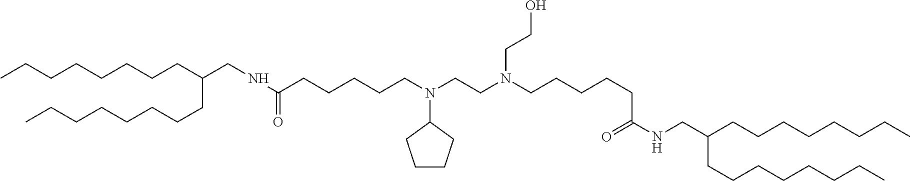 [SMILES: CCCCCCCCC(CCCCCCCC)CNC(=O)CCCCCN(CCO)CCN(CCCCCC(=O)NCC(CCCCCCCC)CCCCCCCC)C1CCCC1]

Compound 12

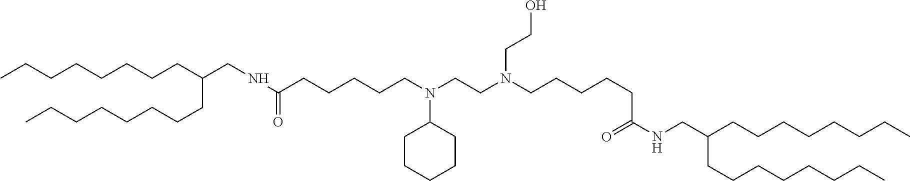 [SMILES: CCCCCCCCC(CCCCCCCC)CNC(=O)CCCCCN(CCO)CCN(CCCCCC(=O)NCC(CCCCCCCC)CCCCCCCC)C1CCCCC1]

Compound 13

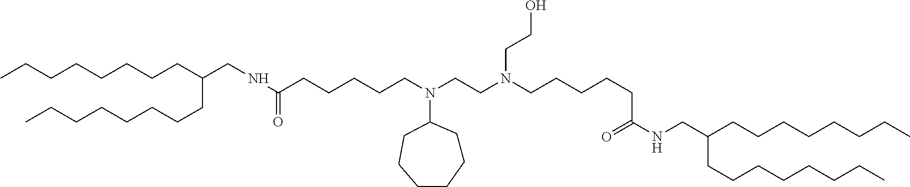 [SMILES: CCCCCCCCC(CCCCCCCC)CNC(=O)CCCCCN(CCO)CCN(CCCCCC(=O)NCC(CCCCCCCC)CCCCCCCC)C1CCCCCC1]

Compound 14

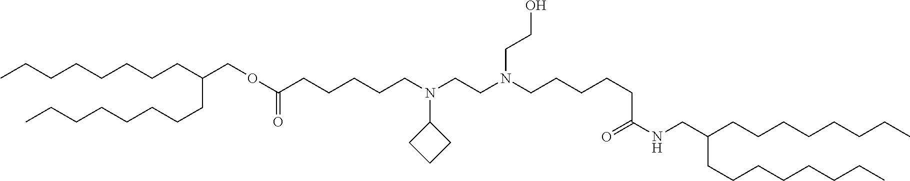 [SMILES: CCCCCCCCC(CCCCCCCC)CNC(=O)CCCCCN(CCO)CCN(CCCCCC(=O)OCC(CCCCCCCC)CCCCCCCC)C1CCC1]

Compound 15

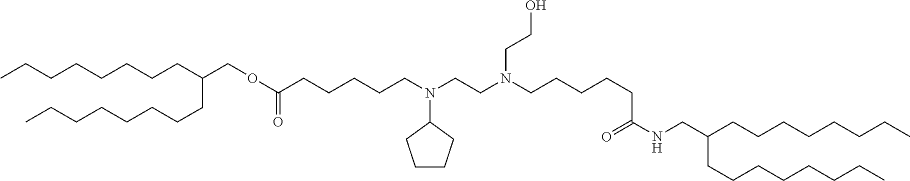 [SMILES: CCCCCCCCC(CCCCCCCC)CNC(=O)CCCCCN(CCO)CCN(CCCCCC(=O)OCC(CCCCCCCC)CCCCCCCC)C1CCCC1]

Compound 16

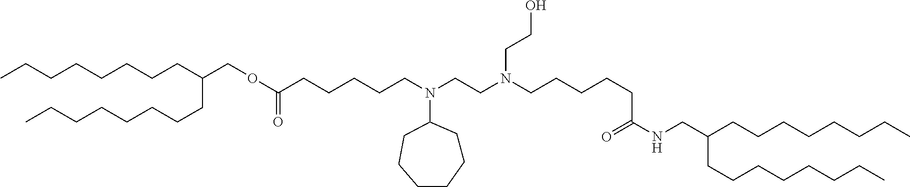 [SMILES: CCCCCCCCC(CCCCCCCC)CNC(=O)CCCCCN(CCO)CCN(CCCCCC(=O)OCC(CCCCCCCC)CCCCCCCC)C1CCCCCC1]

Compound 17

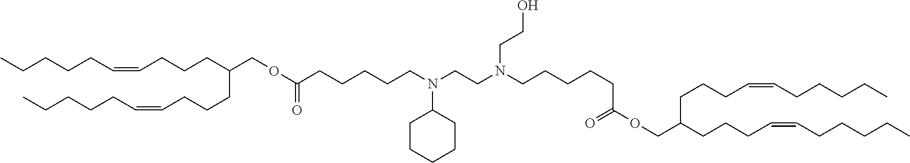 [SMILES: CCCCC/C=C\CCCC(CCC/C=C\CCCCC)COC(=O)CCCCCN(CCO)CCN(CCCCCC(=O)OCC(CCC/C=C\CCCCC)CCC/C=C\CCCCC)C1CCCCC1]

Compound 18

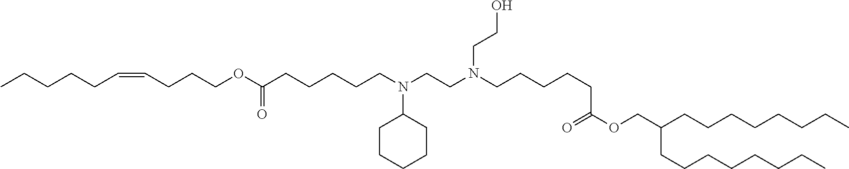 [SMILES: CCCCC/C=C\CCCOC(=O)CCCCCN(CCN(CCO)CCCCCC(=O)OCC(CCCCCCCC)CCCCCCCC)C1CCCCC1]

Compound 19

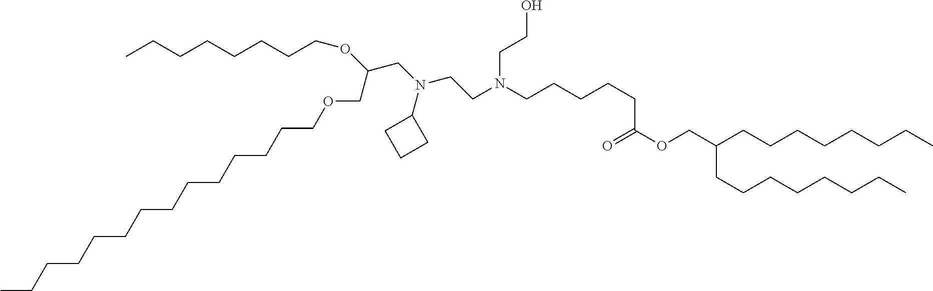 [SMILES: CCCCCCCCCCCCCCOCC(CN(CCN(CCO)CCCCCC(=O)OCC(CCCCCCCC)CCCCCCCC)C1CCC1)OCCCCCCCC]

Compound 20

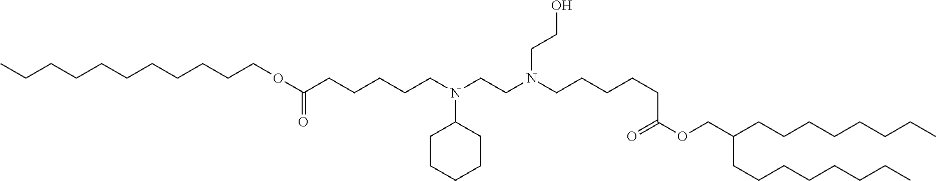 [SMILES: CCCCCCCCCCCOC(=O)CCCCCN(CCN(CCO)CCCCCC(=O)OCC(CCCCCCCC)CCCCCCCC)C1CCCCC1]

Compound 21

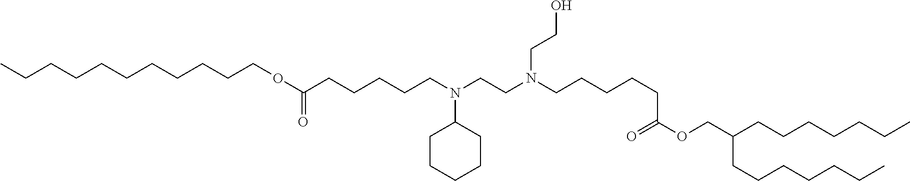 [SMILES: CCCCCCCCCCCOC(=O)CCCCCN(CCN(CCO)CCCCCC(=O)OCC(CCCCCCC)CCCCCCC)C1CCCCC1]

Compound 22

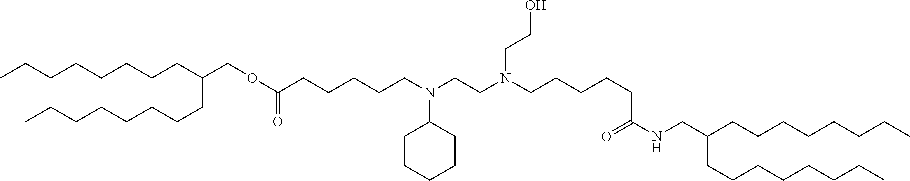 [SMILES: CCCCCCCCC(CCCCCCCC)CNC(=O)CCCCCN(CCO)CCN(CCCCCC(=O)OCC(CCCCCCCC)CCCCCCCC)C1CCCCC1]

Compound 23

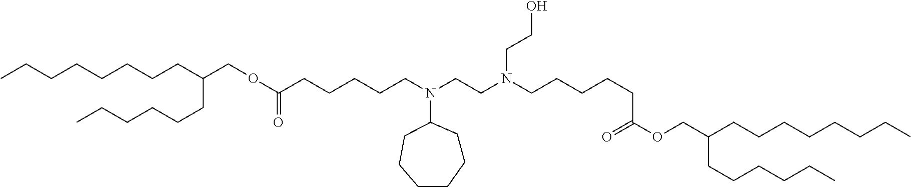 [SMILES: CCCCCCCCC(CCCCCC)COC(=O)CCCCCN(CCO)CCN(CCCCCC(=O)OCC(CCCCCC)CCCCCCCC)C1CCCCCC1]

Compound 24

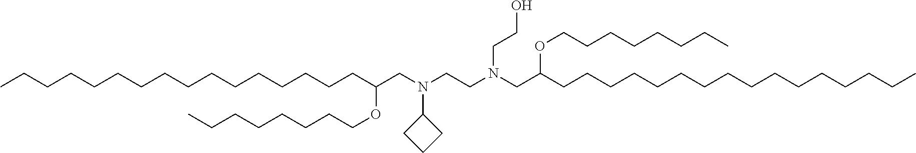 [SMILES: CCCCCCCCCCCCCCCCC(CN(CCO)CCN(CC(CCCCCCCCCCCCCCCC)OCCCCCCCC)C1CCC1)OCCCCCCCC]

Compound 25

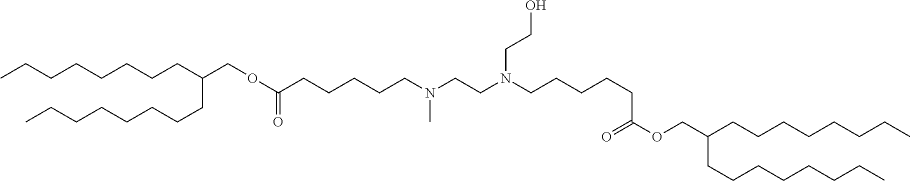 [SMILES: CCCCCCCCC(CCCCCCCC)COC(=O)CCCCCN(C)CCN(CCO)CCCCCC(=O)OCC(CCCCCCCC)CCCCCCCC]

Compound 26

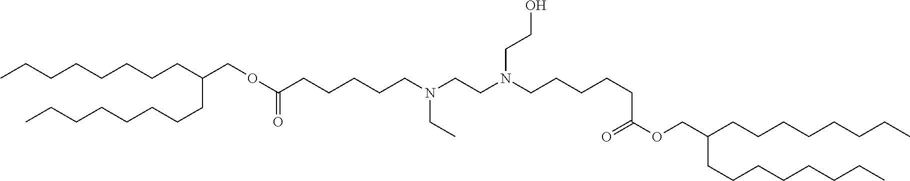 [SMILES: CCCCCCCCC(CCCCCCCC)COC(=O)CCCCCN(CC)CCN(CCO)CCCCCC(=O)OCC(CCCCCCCC)CCCCCCCC]

Compound 27

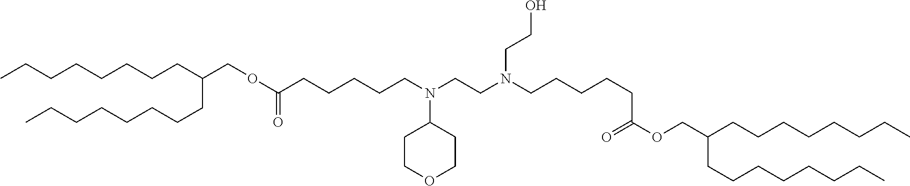 [SMILES: CCCCCCCCC(CCCCCCCC)COC(=O)CCCCCN(CCO)CCN(CCCCCC(=O)OCC(CCCCCCCC)CCCCCCCC)C1CCOCC1]

Compound 28

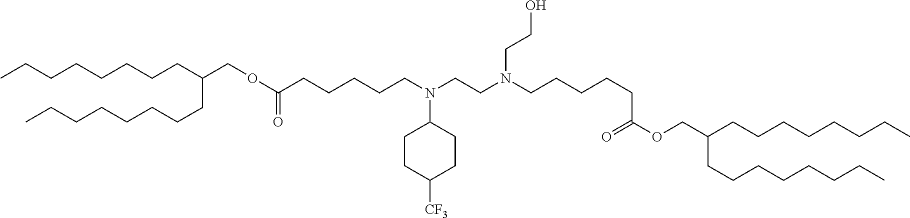 [SMILES: CCCCCCCCC(CCCCCCCC)COC(=O)CCCCCN(CCO)CCN(CCCCCC(=O)OCC(CCCCCCCC)CCCCCCCC)C1CCC(C(F)(F)F)CC1]

Compound 29

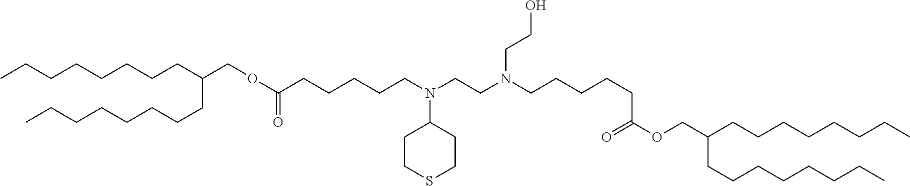 [SMILES: CCCCCCCCC(CCCCCCCC)COC(=O)CCCCCN(CCO)CCN(CCCCCC(=O)OCC(CCCCCCCC)CCCCCCCC)C1CCSCC1]

Compound 30

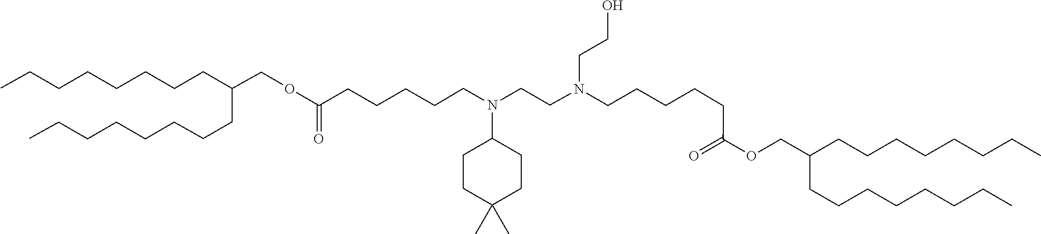 [SMILES: CCCCCCCCC(CCCCCCCC)COC(=O)CCCCCN(CCO)CCN(CCCCCC(=O)OCC(CCCCCCCC)CCCCCCCC)C1CCC(C)(C)CC1]

Compound 31

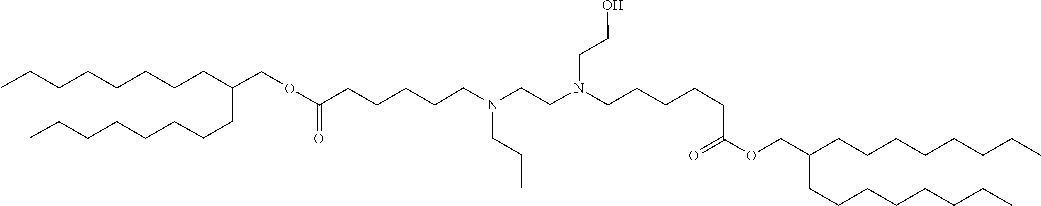 [SMILES: CCCCCCCCC(CCCCCCCC)COC(=O)CCCCCN(CCC)CCN(CCO)CCCCCC(=O)OCC(CCCCCCCC)CCCCCCCC]

Compound 32

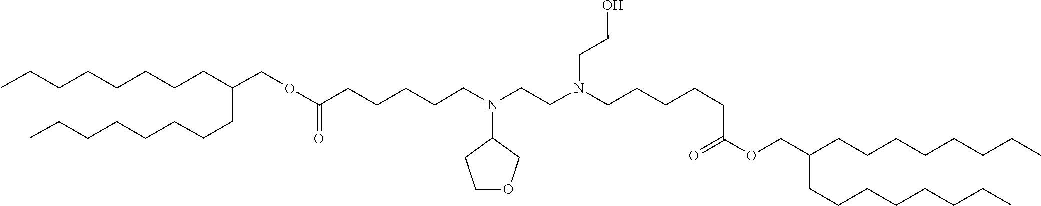 [SMILES: CCCCCCCCC(CCCCCCCC)COC(=O)CCCCCN(CCO)CCN(CCCCCC(=O)OCC(CCCCCCCC)CCCCCCCC)C1CCOC1]

Compound 33

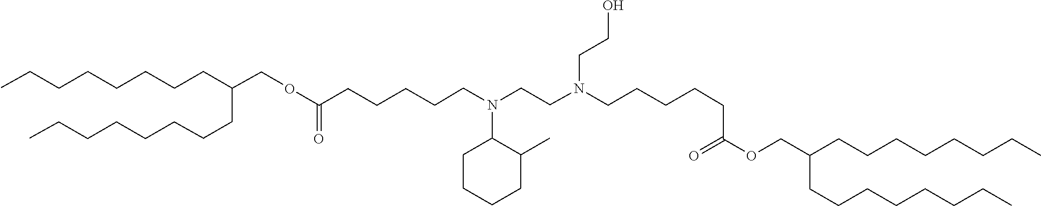 [SMILES: CCCCCCCCC(CCCCCCCC)COC(=O)CCCCCN(CCO)CCN(CCCCCC(=O)OCC(CCCCCCCC)CCCCCCCC)C1CCCCC1C]

Compound 34

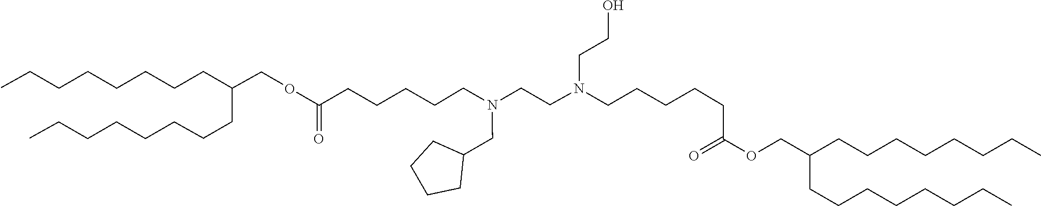 [SMILES: CCCCCCCCC(CCCCCCCC)COC(=O)CCCCCN(CCO)CCN(CCCCCC(=O)OCC(CCCCCCCC)CCCCCCCC)CC1CCCC1]

Compound 35

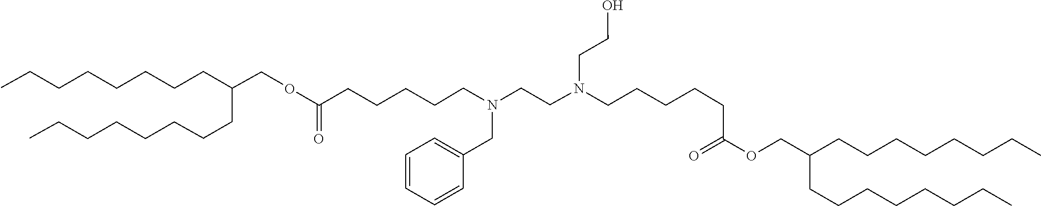 [SMILES: CCCCCCCCC(CCCCCCCC)COC(=O)CCCCCN(CCO)CCN(CCCCCC(=O)OCC(CCCCCCCC)CCCCCCCC)Cc1ccccc1]

Compound 36

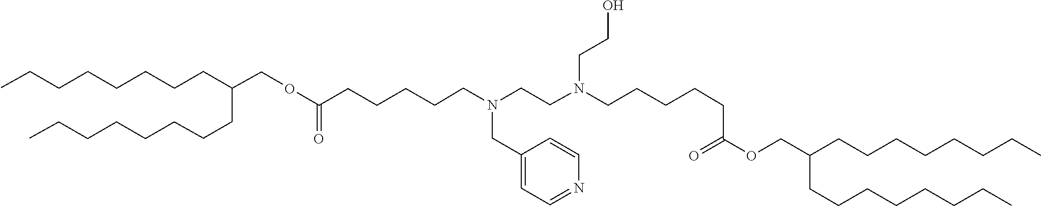 [SMILES: CCCCCCCCC(CCCCCCCC)COC(=O)CCCCCN(CCO)CCN(CCCCCC(=O)OCC(CCCCCCCC)CCCCCCCC)Cc1ccncc1]

Compound 37

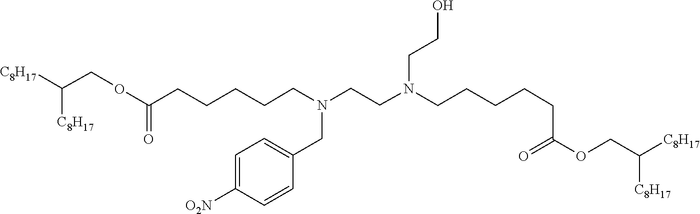

Compound 38

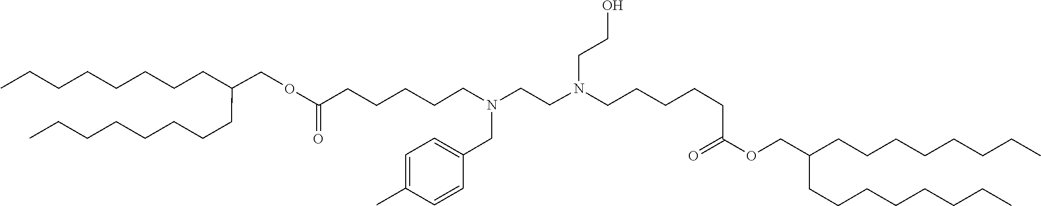 [SMILES: CCCCCCCCC(CCCCCCCC)COC(=O)CCCCCN(CCO)CCN(CCCCCC(=O)OCC(CCCCCCCC)CCCCCCCC)Cc1ccc(C)cc1]

Compound 39

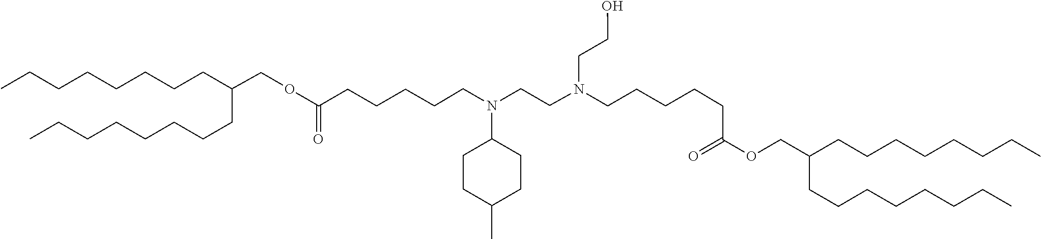 [SMILES: CCCCCCCCC(CCCCCCCC)COC(=O)CCCCCN(CCO)CCN(CCCCCC(=O)OCC(CCCCCCCC)CCCCCCCC)C1CCC(C)CC1]

Compound 40

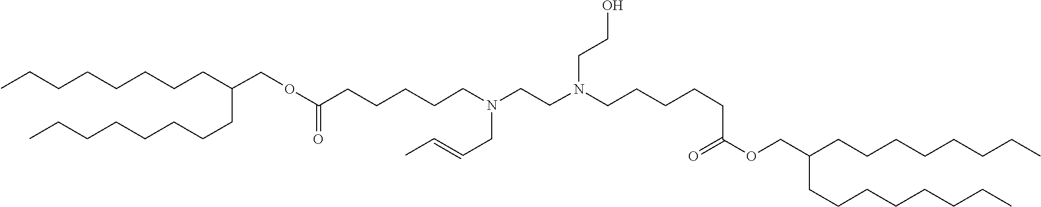 [SMILES: C/C=C/CN(CCCCCC(=O)OCC(CCCCCCCC)CCCCCCCC)CCN(CCO)CCCCCC(=O)OCC(CCCCCCCC)CCCCCCCC]

Compound 41

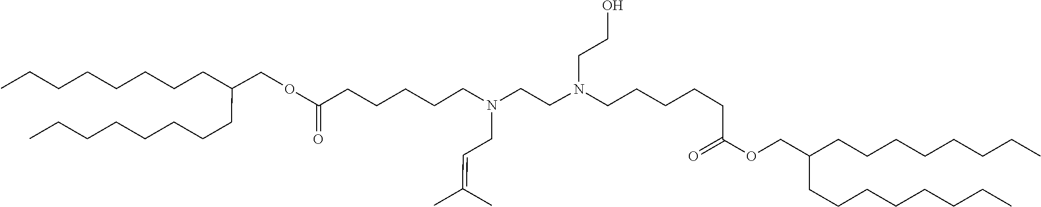 [SMILES: CCCCCCCCC(CCCCCCCC)COC(=O)CCCCCN(CC=C(C)C)CCN(CCO)CCCCCC(=O)OCC(CCCCCCCC)CCCCCCCC]

Compound 42

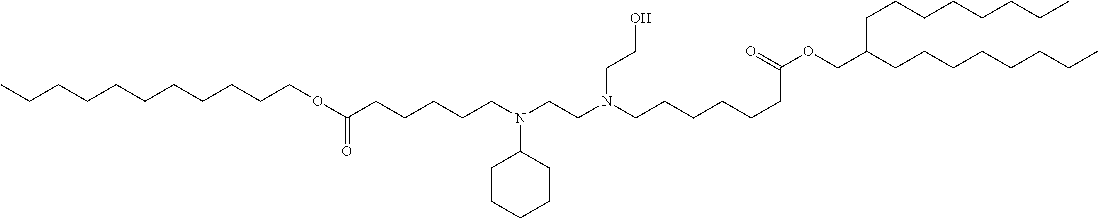 [SMILES: CCCCCCCCCCCOC(=O)CCCCCN(CCN(CCO)CCCCCCC(=O)OCC(CCCCCCCC)CCCCCCCC)C1CCCCC1]

Compound 43

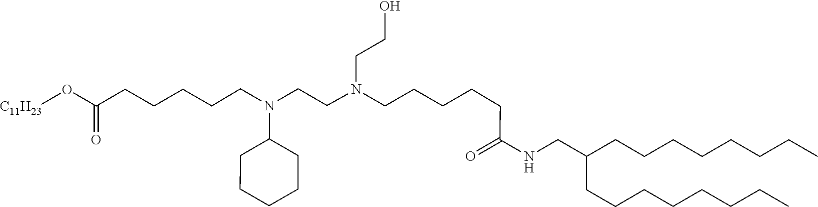

Compound 44

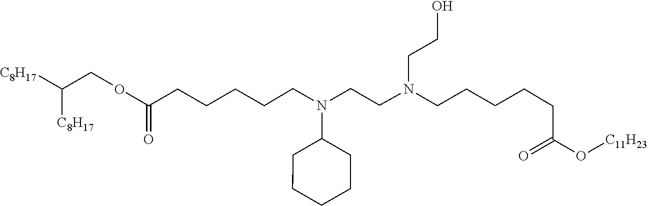

Compound 45

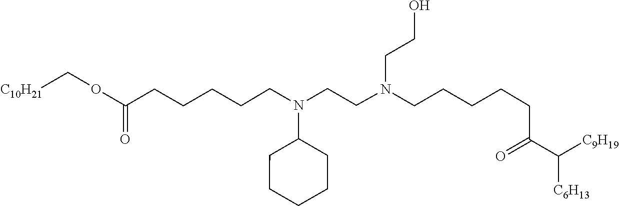

Compound 46

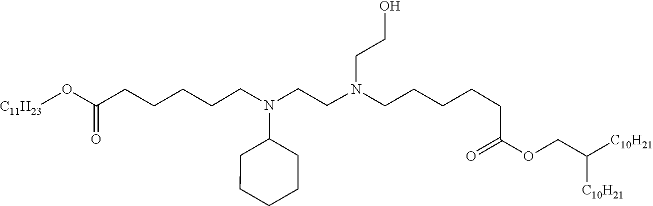

Compound 47

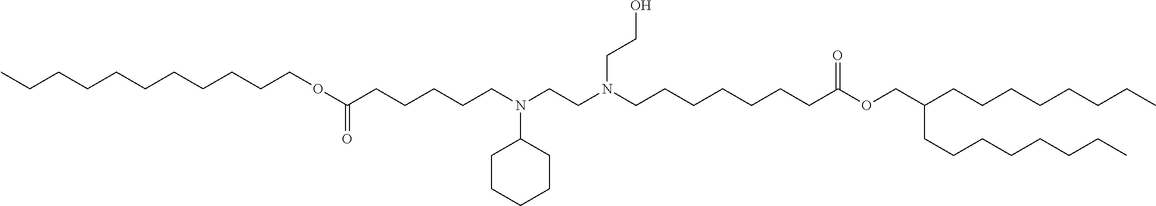 [SMILES: CCCCCCCCCCCOC(=O)CCCCCN(CCN(CCO)CCCCCCCC(=O)OCC(CCCCCCCC)CCCCCCCC)C1CCCCC1]

Compound 48

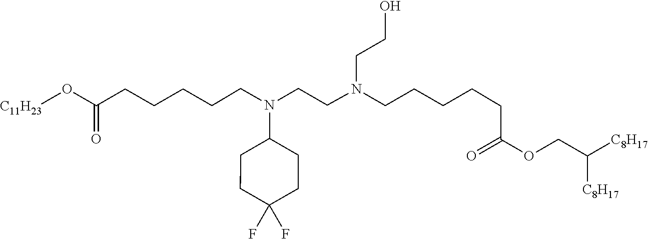

Compound 49

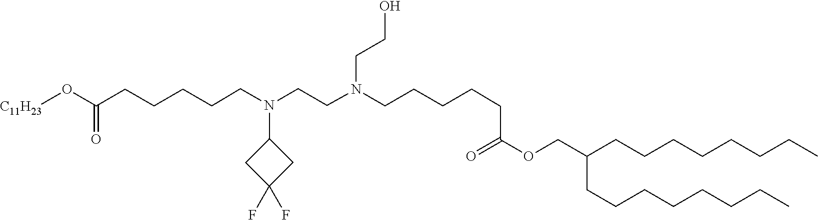

Compound 50

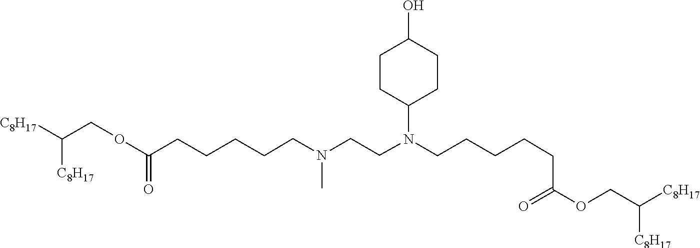

Compound 51

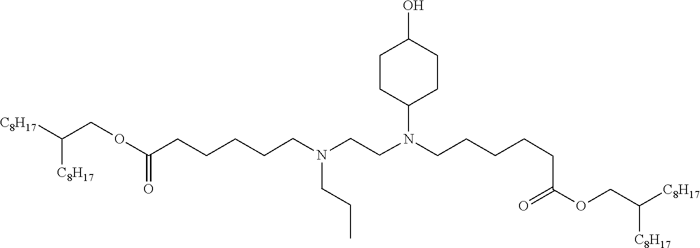

Compound 52

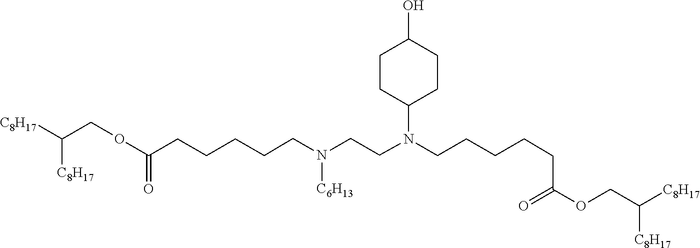

Compound 53

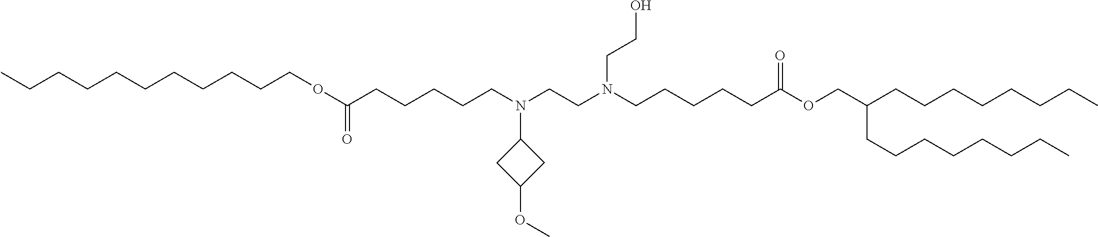 [SMILES: CCCCCCCCCCCOC(=O)CCCCCN(CCN(CCO)CCCCCC(=O)OCC(CCCCCCCC)CCCCCCCC)C1CC(OC)C1]

Compound 54

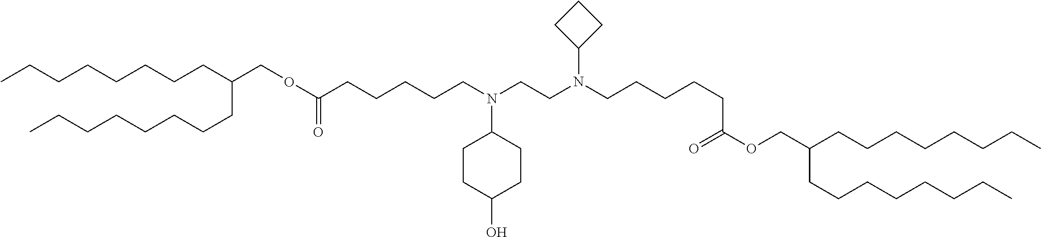 [SMILES: CCCCCCCCC(CCCCCCCC)COC(=O)CCCCCN(CCN(CCCCCC(=O)OCC(CCCCCCCC)CCCCCCCC)C1CCC(O)CC1)C1CCC1]

Compound 55

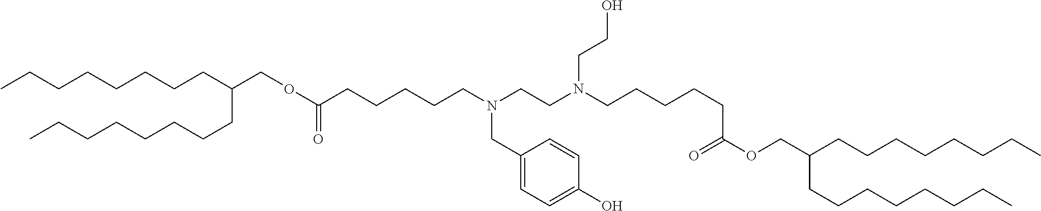 [SMILES: CCCCCCCCC(CCCCCCCC)COC(=O)CCCCCN(CCO)CCN(CCCCCC(=O)OCC(CCCCCCCC)CCCCCCCC)Cc1ccc(O)cc1]

Compound 56

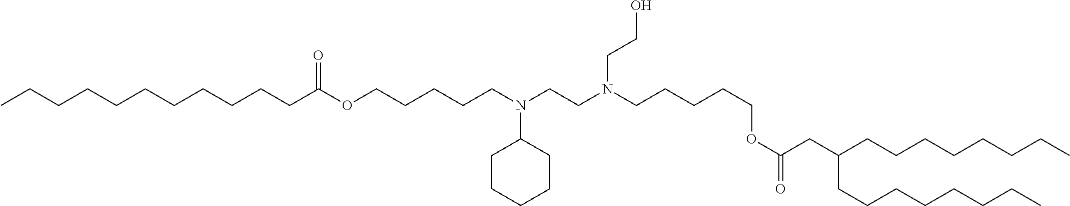 [SMILES: CCCCCCCCCCCC(=O)OCCCCCN(CCN(CCO)CCCCCOC(=O)CC(CCCCCCCC)CCCCCCCC)C1CCCCC1]

Compound 57

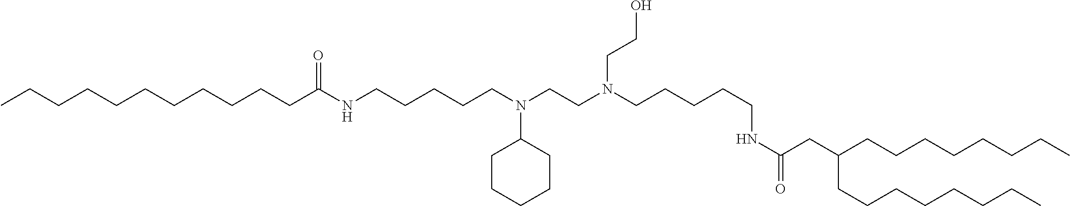 [SMILES: CCCCCCCCCCCC(=O)NCCCCCN(CCN(CCO)CCCCCNC(=O)CC(CCCCCCCC)CCCCCCCC)C1CCCCC1]

Compound 58

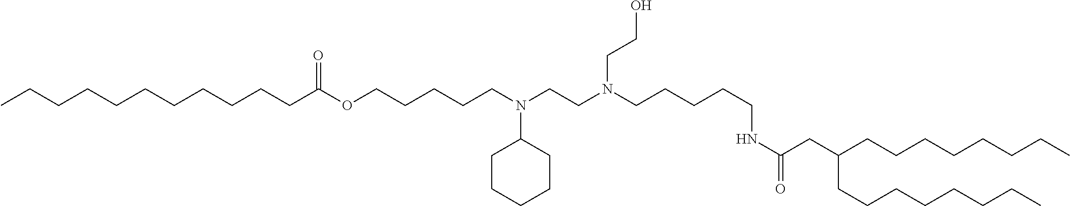 [SMILES: CCCCCCCCCCCC(=O)OCCCCCN(CCN(CCO)CCCCCNC(=O)CC(CCCCCCCC)CCCCCCCC)C1CCCCC1]

Compound 59

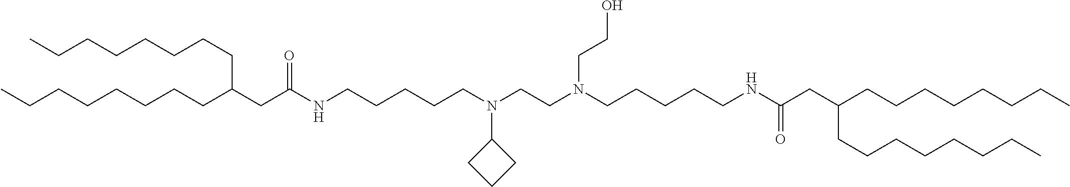 [SMILES: CCCCCCCCC(CCCCCCCC)CC(=O)NCCCCCN(CCO)CCN(CCCCCNC(=O)CC(CCCCCCCC)CCCCCCCC)C1CCC1]

Compound 60

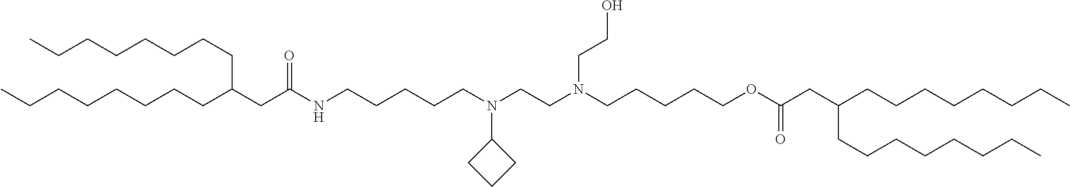 [SMILES: CCCCCCCCC(CCCCCCCC)CC(=O)NCCCCCN(CCN(CCO)CCCCCOC(=O)CC(CCCCCCCC)CCCCCCCC)C1CCC1]

Compound 61

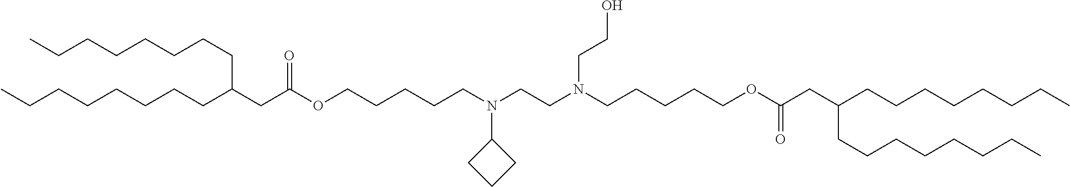 [SMILES: CCCCCCCCC(CCCCCCCC)CC(=O)OCCCCCN(CCO)CCN(CCCCCOC(=O)CC(CCCCCCCC)CCCCCCCC)C1CCC1]

Compound 62

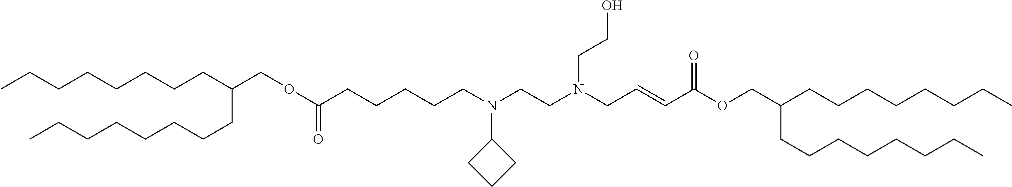 [SMILES: CCCCCCCCC(CCCCCCCC)COC(=O)/C=C/CN(CCO)CCN(CCCCCC(=O)OCC(CCCCCCCC)CCCCCCCC)C1CCC1]

Compound 63

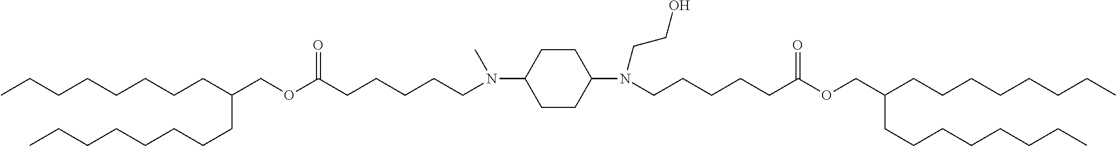 [SMILES: CCCCCCCCC(CCCCCCCC)COC(=O)CCCCCN(C)C1CCC(N(CCO)CCCCCC(=O)OCC(CCCCCCCC)CCCCCCCC)CC1]

Compound 64

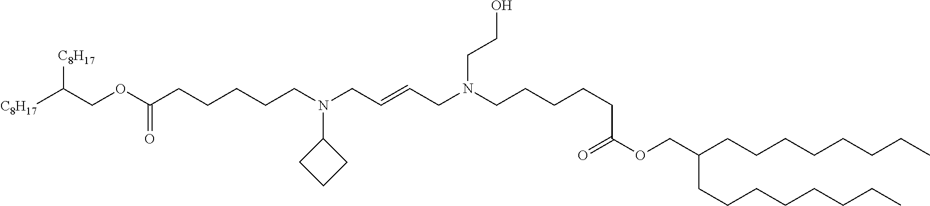

Compound 65

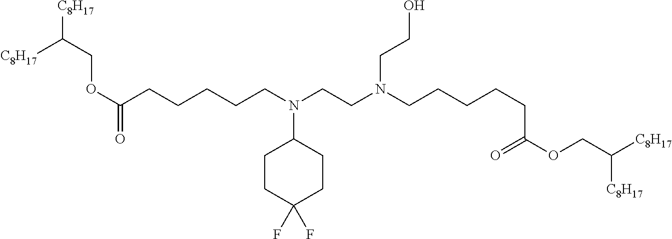

Compound 66

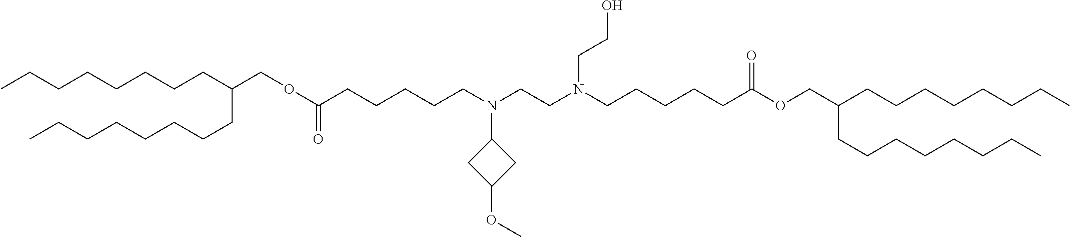 [SMILES: CCCCCCCCC(CCCCCCCC)COC(=O)CCCCCN(CCO)CCN(CCCCCC(=O)OCC(CCCCCCCC)CCCCCCCC)C1CC(OC)C1]

Compound 67

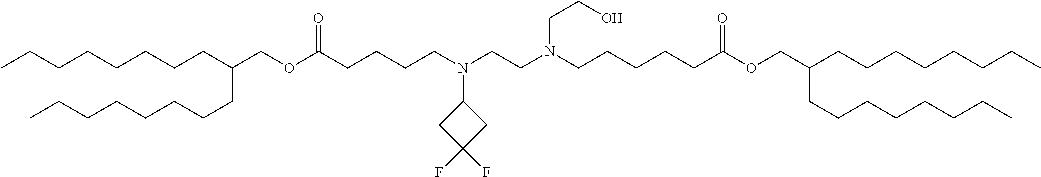 [SMILES: CCCCCCCCC(CCCCCCCC)COC(=O)CCCCCN(CCO)CCN(CCCCC(=O)OCC(CCCCCCCC)CCCCCCCC)C1CC(F)(F)C1]

Compound 68

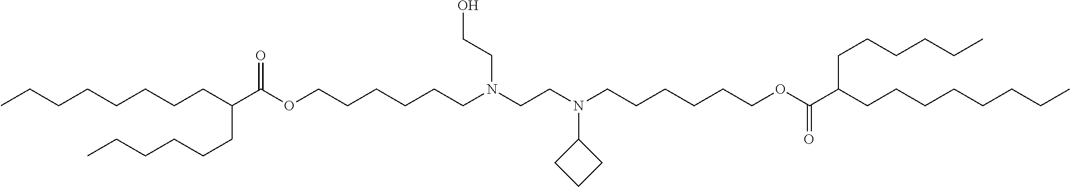 [SMILES: CCCCCCCCC(CCCCCC)C(=O)OCCCCCCN(CCO)CCN(CCCCCCOC(=O)C(CCCCCC)CCCCCCCC)C1CCC1]

Compound 69

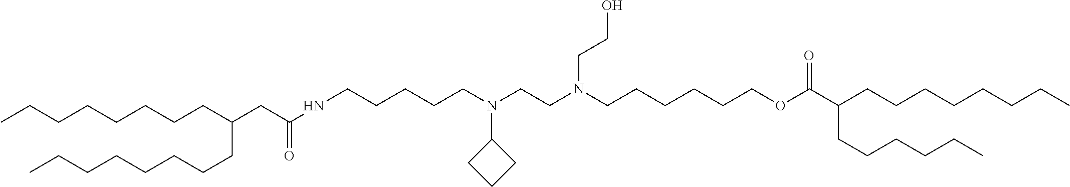 [SMILES: CCCCCCCCC(CCCCCCCC)CC(=O)NCCCCCN(CCN(CCO)CCCCCCOC(=O)C(CCCCCC)CCCCCCCC)C1CCC1]

Compound 70

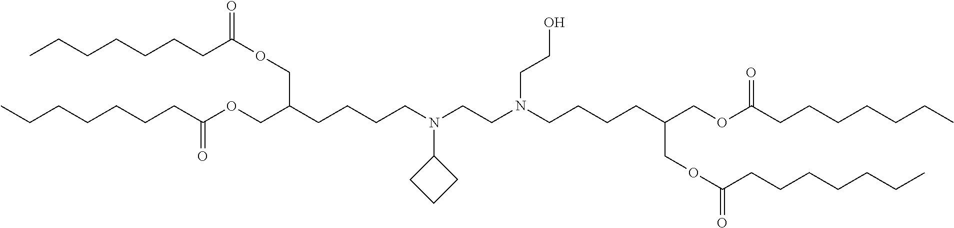 [SMILES: CCCCCCCC(=O)OCC(CCCCN(CCO)CCN(CCCCC(COC(=O)CCCCCCC)COC(=O)CCCCCCC)C1CCC1)COC(=O)CCCCCCC]

Compound 71

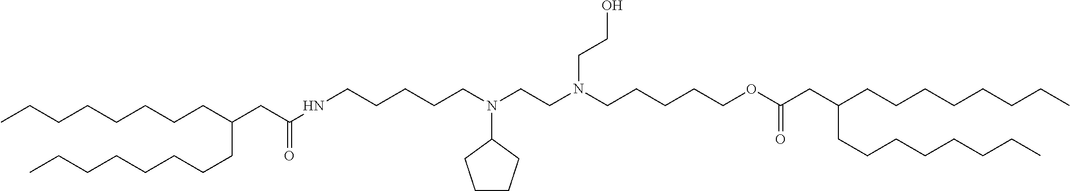 [SMILES: CCCCCCCCC(CCCCCCCC)CC(=O)NCCCCCN(CCN(CCO)CCCCCOC(=O)CC(CCCCCCCC)CCCCCCCC)C1CCCC1]

Compound 72

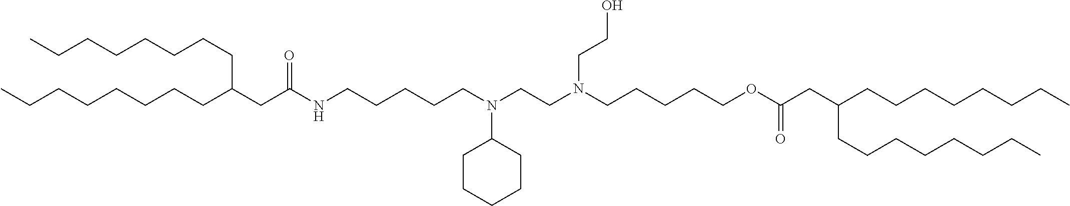 [SMILES: CCCCCCCCC(CCCCCCCC)CC(=O)NCCCCCN(CCN(CCO)CCCCCOC(=O)CC(CCCCCCCC)CCCCCCCC)C1CCCCC1]

Compound 73

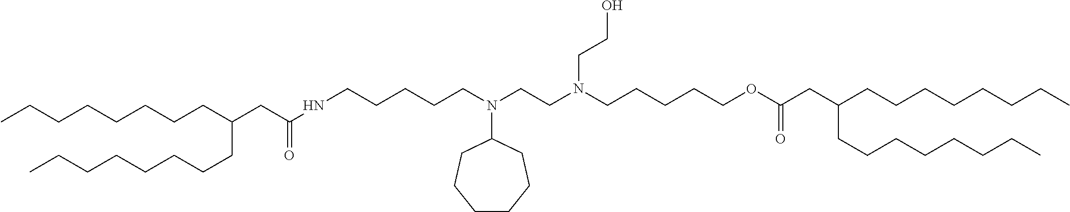 [SMILES: CCCCCCCCC(CCCCCCCC)CC(=O)NCCCCCN(CCN(CCO)CCCCCOC(=O)CC(CCCCCCCC)CCCCCCCC)C1CCCCCC1]

Compound 74

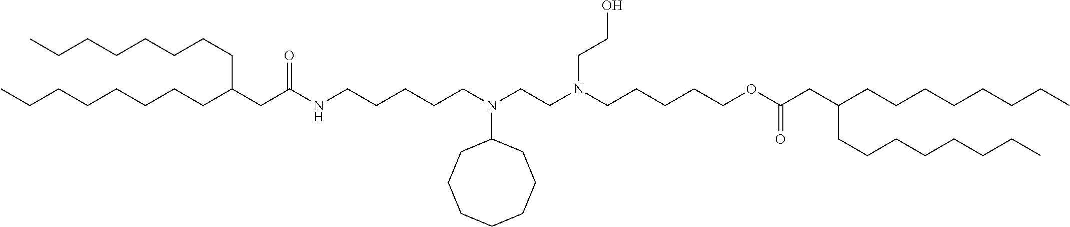 [SMILES: CCCCCCCCC(CCCCCCCC)CC(=O)NCCCCCN(CCN(CCO)CCCCCOC(=O)CC(CCCCCCCC)CCCCCCCC)C1CCCCCCC1]

Compound 75

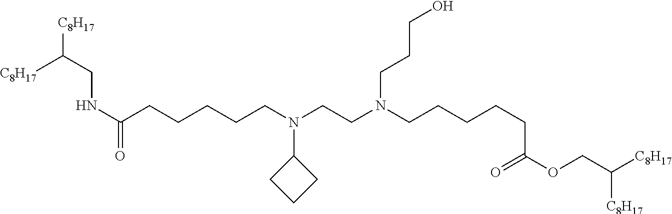

Compound 76

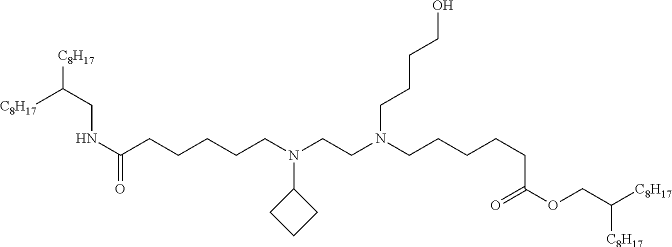

Compound 77

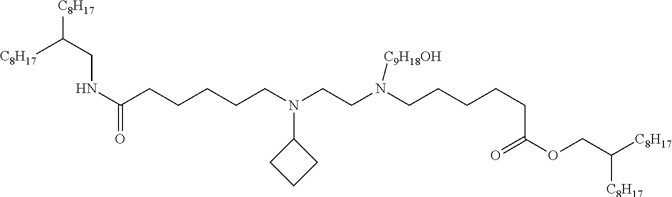

Compound 78

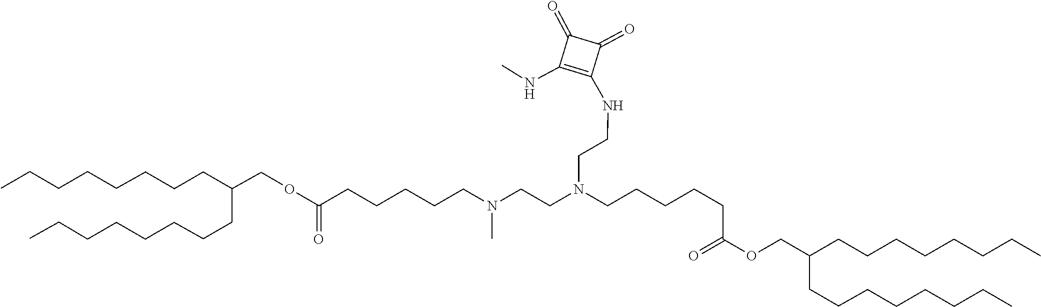 [SMILES: CCCCCCCCC(CCCCCCCC)COC(=O)CCCCCN(C)CCN(CCCCCC(=O)OCC(CCCCCCCC)CCCCCCCC)CCNc1c(NC)c(=O)c1=O]

Compound 79

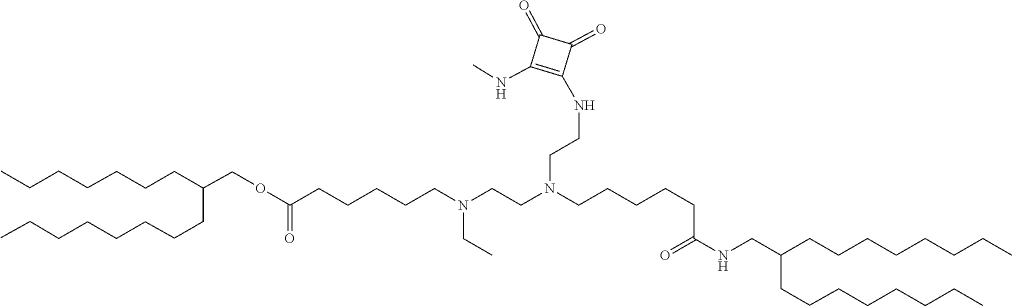 [SMILES: CCCCCCCCC(CCCCCCCC)CNC(=O)CCCCCN(CCNc1c(NC)c(=O)c1=O)CCN(CC)CCCCCC(=O)OCC(CCCCCCC)CCCCCCCC]

Compound 80

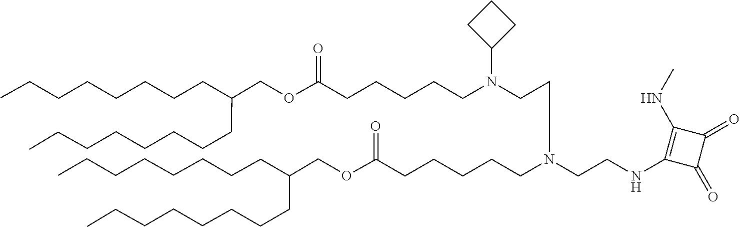 [SMILES: CCCCCCCCC(CCCCCCCC)COC(=O)CCCCCN(CCNc1c(NC)c(=O)c1=O)CCN(CCCCCC(=O)OCC(CCCCCCCC)CCCCCCCC)C1CCC1]

Compound 81

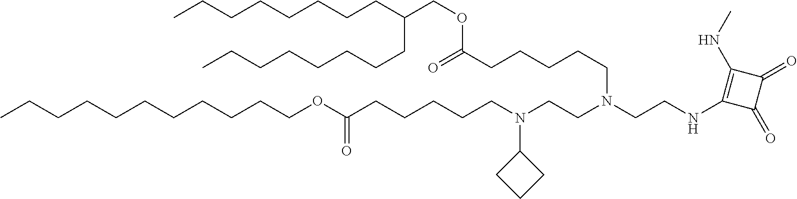 [SMILES: CCCCCCCCCCCOC(=O)CCCCCN(CCN(CCCCCC(=O)OCC(CCCCCCCC)CCCCCCCC)CCNc1c(NC)c(=O)c1=O)C1CCC1]

Compound 82

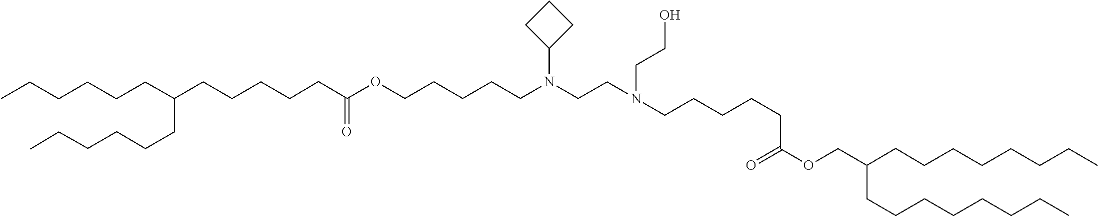 [SMILES: CCCCCCCCC(CCCCCCCC)COC(=O)CCCCCN(CCO)CCN(CCCCCOC(=O)CCCCCC(CCCCCC)CCCCCC)C1CCC1]

Compound 83

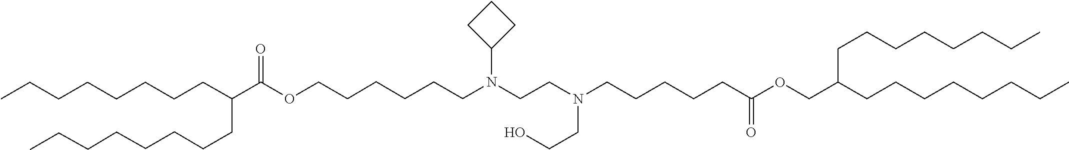 [SMILES: CCCCCCCCC(CCCCCCCC)COC(=O)CCCCCN(CCO)CCN(CCCCCCOC(=O)C(CCCCCCCC)CCCCCCCC)C1CCC1]

Compound 84

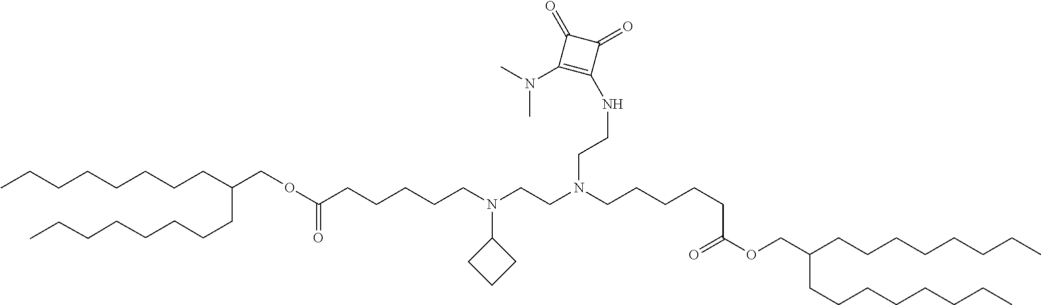 [SMILES: CCCCCCCCC(CCCCCCCC)COC(=O)CCCCCN(CCNc1c(N(C)C)c(=O)c1=O)CCN(CCCCCC(=O)OCC(CCCCCCCC)CCCCCCCC)C1CCC1]

Compound 85

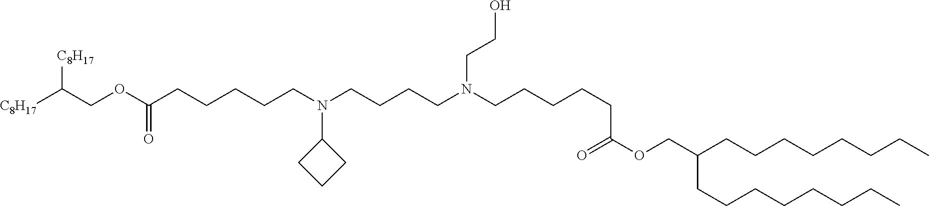

Compound 86

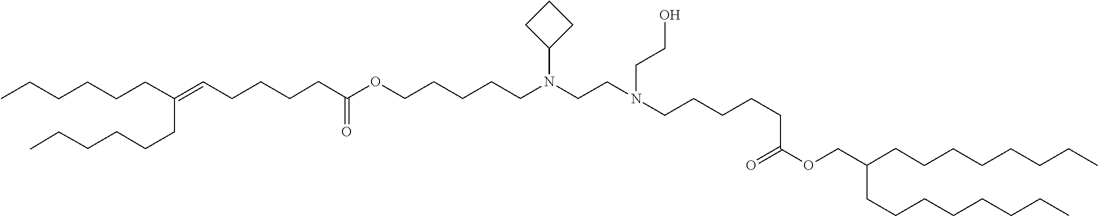 [SMILES: CCCCCCCCC(CCCCCCCC)COC(=O)CCCCCN(CCO)CCN(CCCCCOC(=O)CCCCC=C(CCCCCC)CCCCCC)C1CCC1]

Compound 87

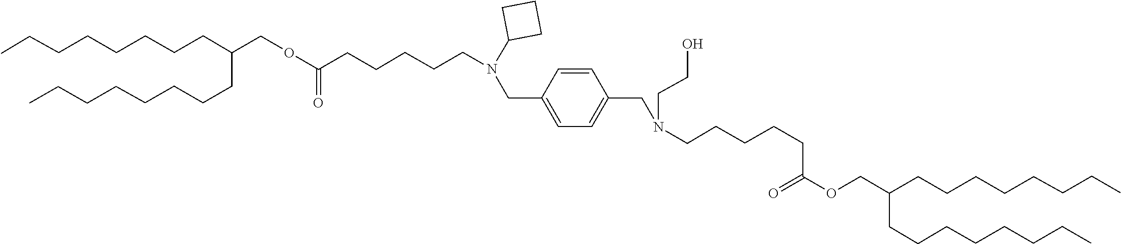 [SMILES: CCCCCCCCC(CCCCCCCC)COC(=O)CCCCCN(CCO)Cc1ccc(CN(CCCCCC(=O)OCC(CCCCCCCC)CCCCCCCC)C2CCC2)cc1]

Compound 88

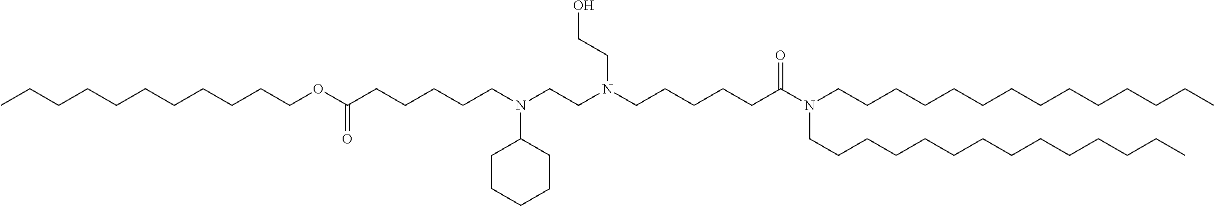 [SMILES: CCCCCCCCCCCCCCN(CCCCCCCCCCCCCC)C(=O)CCCCCN(CCO)CCN(CCCCCC(=O)OCCCCCCCCCCC)C1CCCCC1]

Compound 89

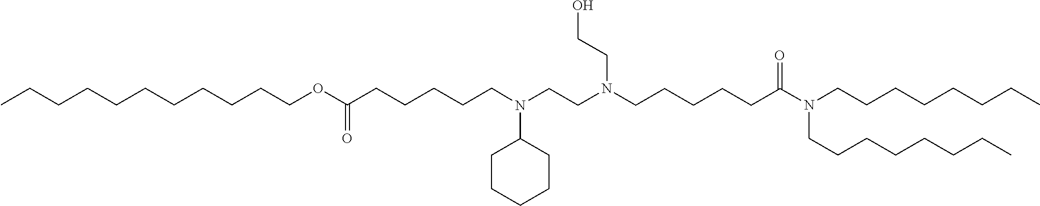 [SMILES: CCCCCCCCCCCOC(=O)CCCCCN(CCN(CCO)CCCCCC(=O)N(CCCCCCCC)CCCCCCCC)C1CCCCC1]

Compound 90

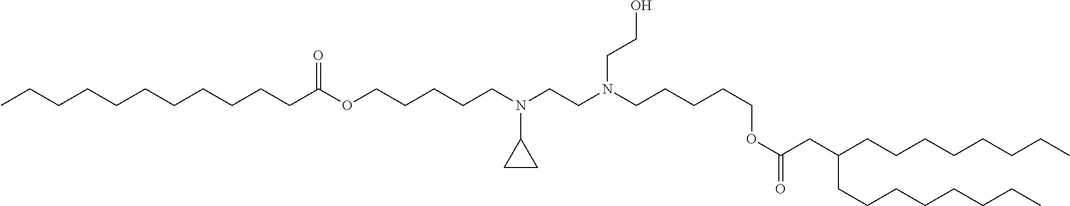 [SMILES: CCCCCCCCCCCC(=O)OCCCCCN(CCN(CCO)CCCCCOC(=O)CC(CCCCCCCC)CCCCCCCC)C1CC1]

Compound 91

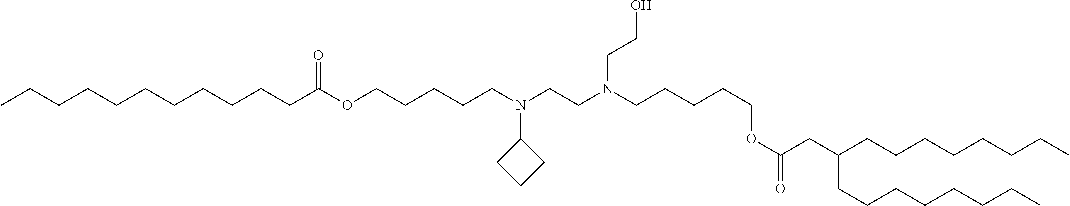 [SMILES: CCCCCCCCCCCC(=O)OCCCCCN(CCN(CCO)CCCCCOC(=O)CC(CCCCCCCC)CCCCCCCC)C1CCC1]

Compound 92

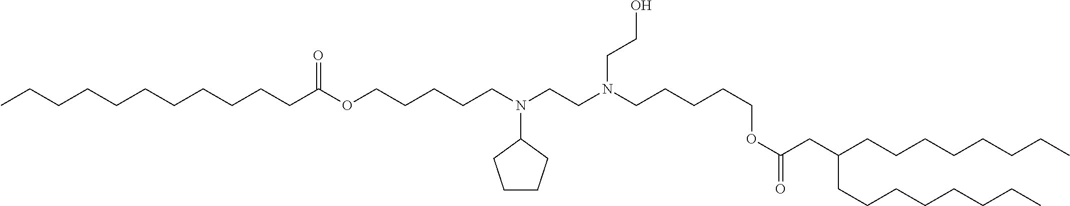 [SMILES: CCCCCCCCCCCC(=O)OCCCCCN(CCN(CCO)CCCCCOC(=O)CC(CCCCCCCC)CCCCCCCC)C1CCCC1]

Compound 93

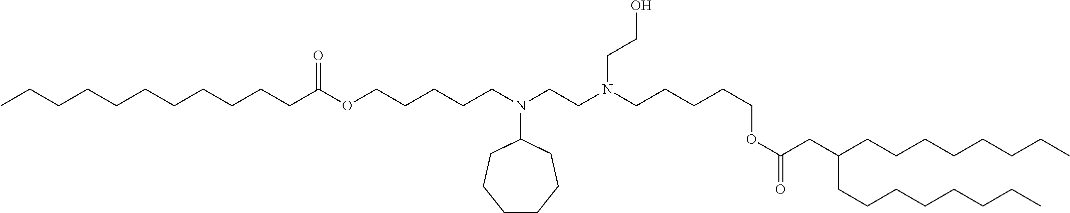 [SMILES: CCCCCCCCCCCC(=O)OCCCCCN(CCN(CCO)CCCCCOC(=O)CC(CCCCCCCC)CCCCCCCC)C1CCCCCC1]

Compound 94

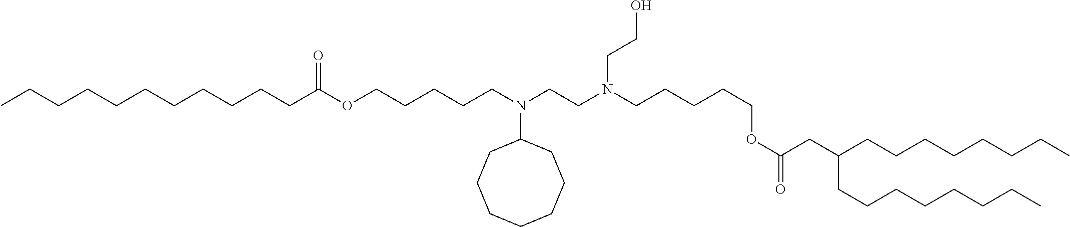 [SMILES: CCCCCCCCCCCC(=O)OCCCCCN(CCN(CCO)CCCCCOC(=O)CC(CCCCCCCC)CCCCCCCC)C1CCCCCCC1]

Compound 95

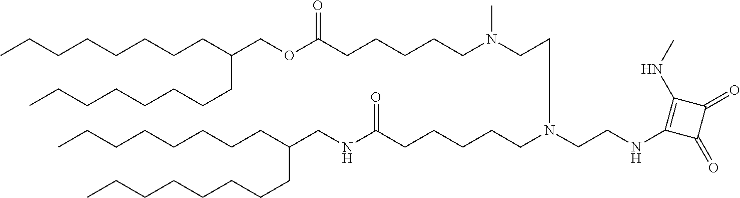 [SMILES: CCCCCCCCC(CCCCCCCC)CNC(=O)CCCCCN(CCNc1c(NC)c(=O)c1=O)CCN(C)CCCCCC(=O)OCC(CCCCCCCC)CCCCCCCC]

Compound 96

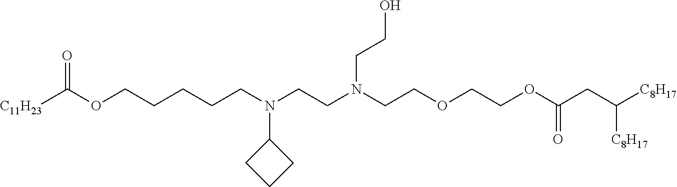

Compound 97

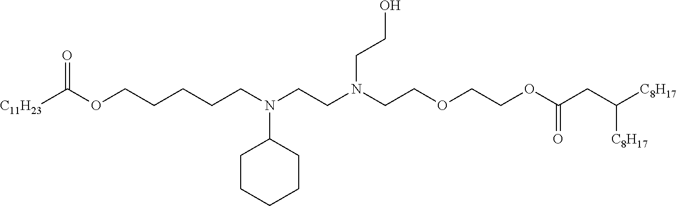

Compound 98

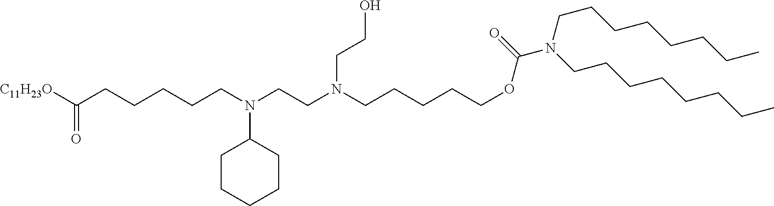

Compound 99

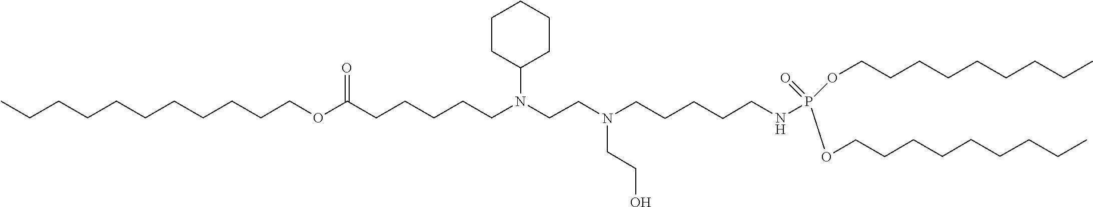 [SMILES: CCCCCCCCCCCOC(=O)CCCCCN(CCN(CCO)CCCCCNP(=O)(OCCCCCCCCC)OCCCCCCCCC)C1CCCCC1]

Compound 100

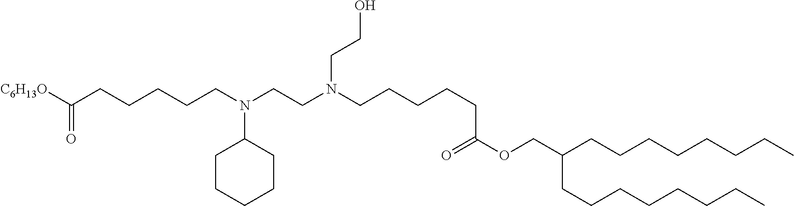

Compound 101

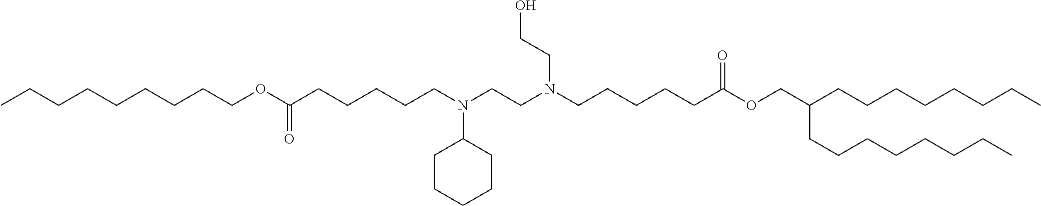 [SMILES: CCCCCCCCCOC(=O)CCCCCN(CCN(CCO)CCCCCC(=O)OCC(CCCCCCCC)CCCCCCCC)C1CCCCC1]

Compound 102

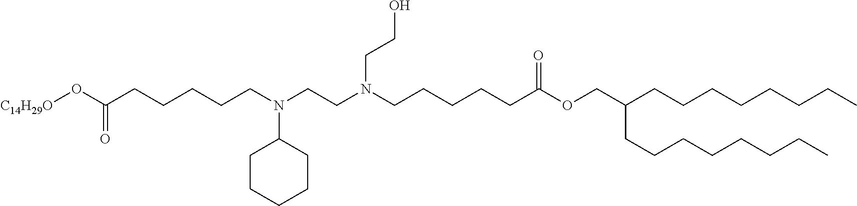

Compound 103

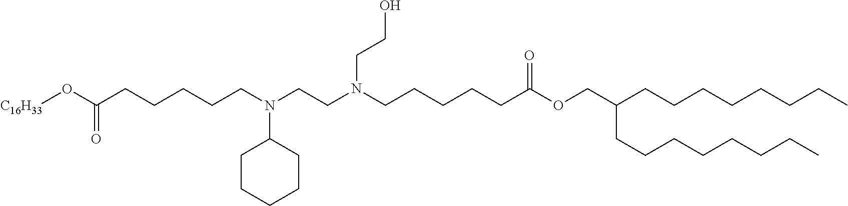

Compound 104

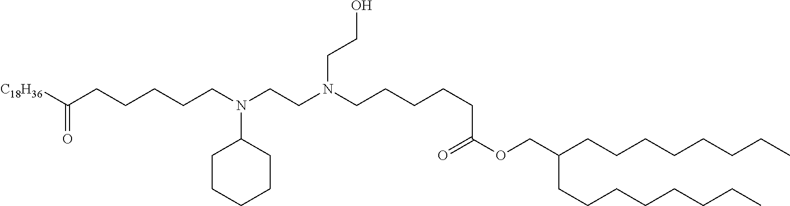

Compound 105

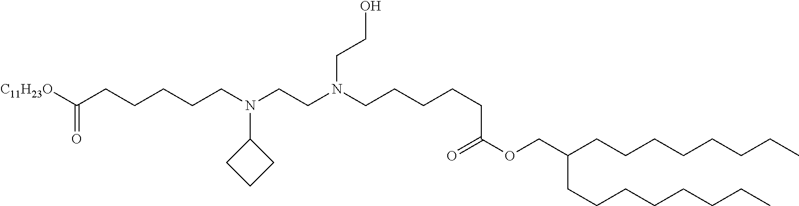

Compound 106

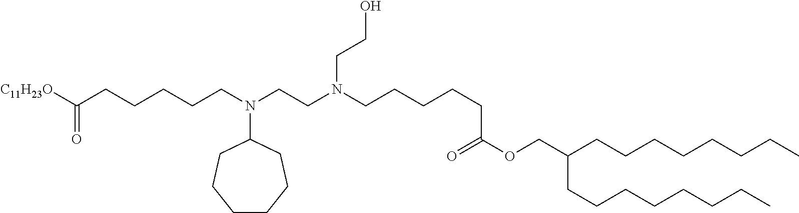

Compound 107

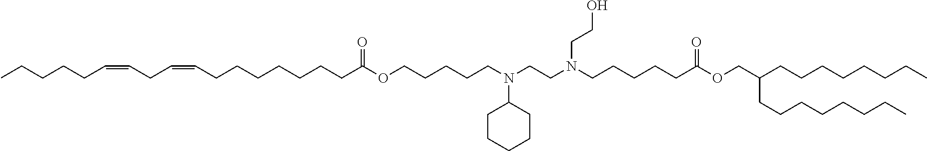 [SMILES: CCCCC/C=C\C/C=C\CCCCCCCC(=O)OCCCCCN(CCN(CCO)CCCCCC(=O)OCC(CCCCCCCC)CCCCCCCC)C1CCCCC1]

Compound 108

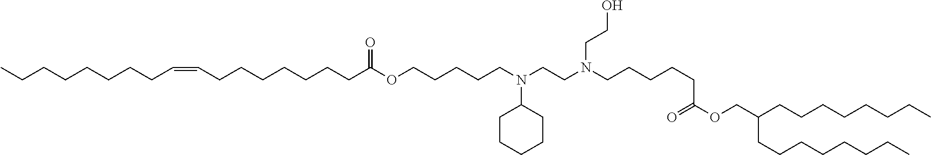 [SMILES: CCCCCCCC/C=C\CCCCCCCC(=O)OCCCCCN(CCN(CCO)CCCCCC(=O)OCC(CCCCCCCC)CCCCCCCC)C1CCCCC1]

Compound 109

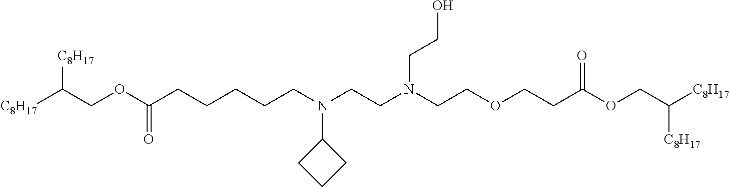

Compound 110

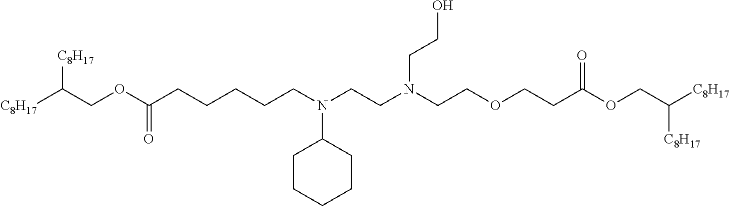

Compound 111

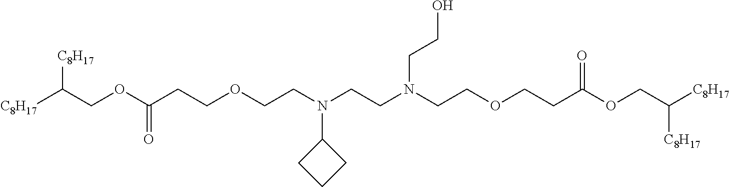

Compound 112

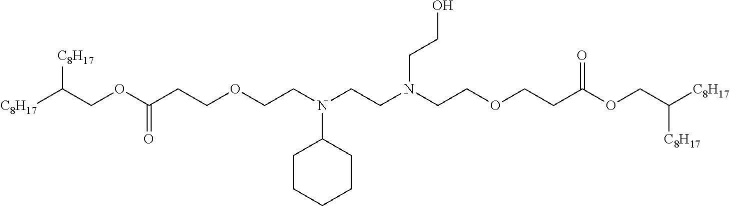

Compound 113

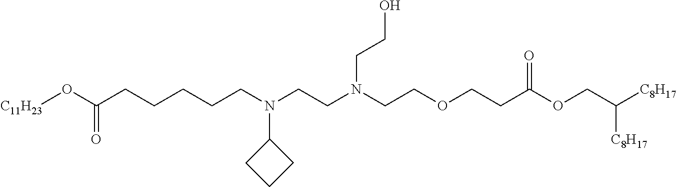

Compound 114

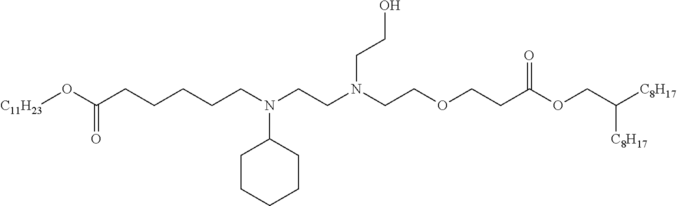

Compound 115

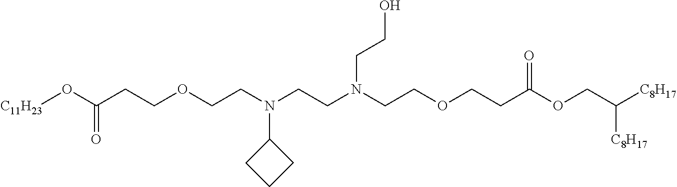

Compound 116

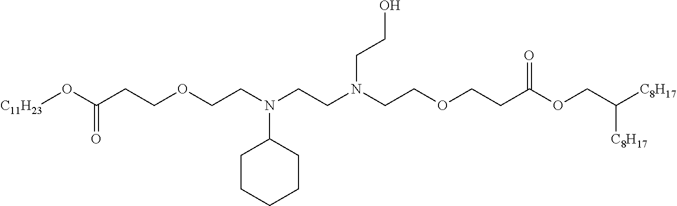

Compound 117

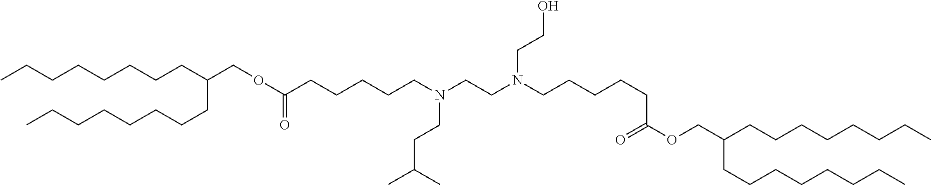 [SMILES: CCCCCCCCC(CCCCCCCC)COC(=O)CCCCCN(CCO)CCN(CCCCCC(=O)OCC(CCCCCCCC)CCCCCCCC)CCC(C)C]

Compound 118

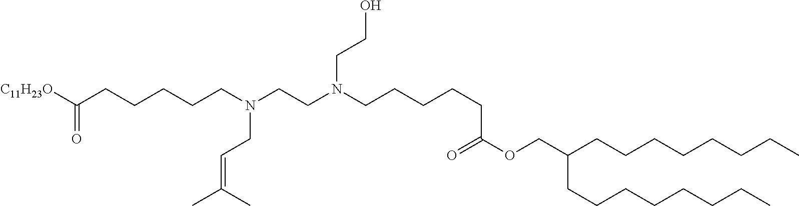

Compound 119

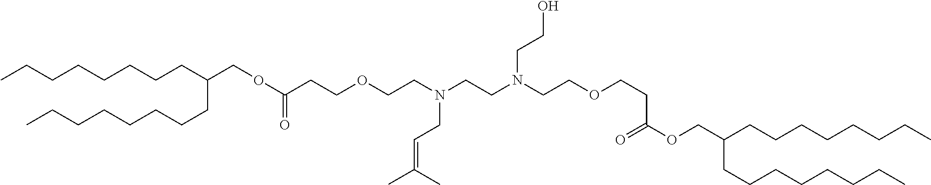 [SMILES: CCCCCCCCC(CCCCCCCC)COC(=O)CCOCCN(CC=C(C)C)CCN(CCO)CCOCCC(=O)OCC(CCCCCCCC)CCCCCCCC]

Compound 120

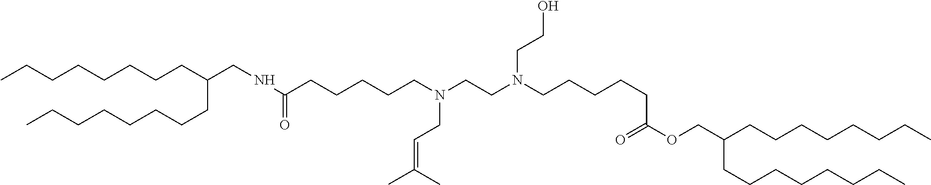 [SMILES: CCCCCCCCC(CCCCCCCC)CNC(=O)CCCCCN(CC=C(C)C)CCN(CCO)CCCCCC(=O)OCC(CCCCCCCC)CCCCCCCC]

Compound 121

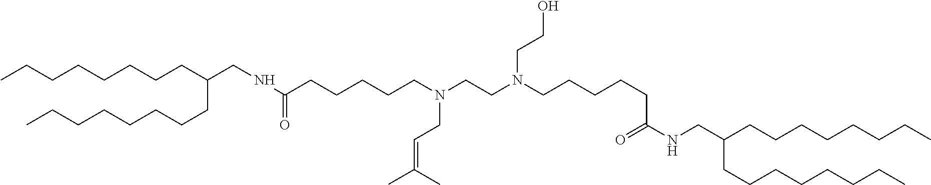 [SMILES: CCCCCCCCC(CCCCCCCC)CNC(=O)CCCCCN(CC=C(C)C)CCN(CCO)CCCCCC(=O)NCC(CCCCCCCC)CCCCCCCC]

Compound 122

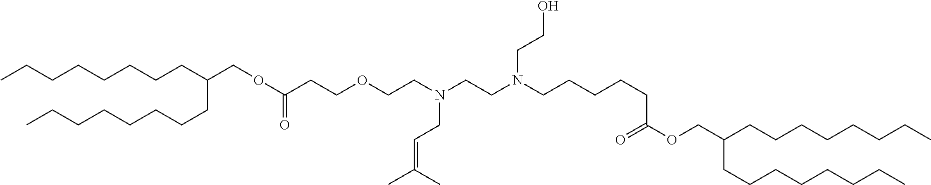 [SMILES: CCCCCCCCC(CCCCCCCC)COC(=O)CCCCCN(CCO)CCN(CC=C(C)C)CCOCCC(=O)OCC(CCCCCCCC)CCCCCCCC]

Compound 123

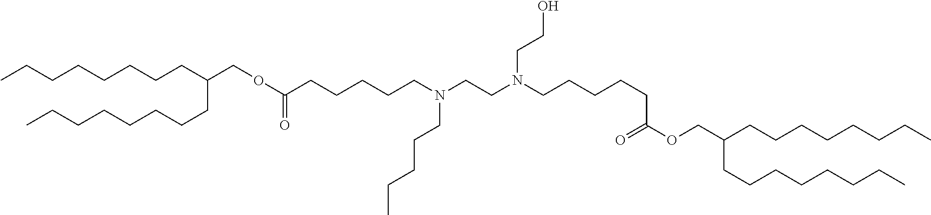 [SMILES: CCCCCCCCC(CCCCCCCC)COC(=O)CCCCCN(CCO)CCN(CCCCC)CCCCCC(=O)OCC(CCCCCCCC)CCCCCCCC]

Compound 124

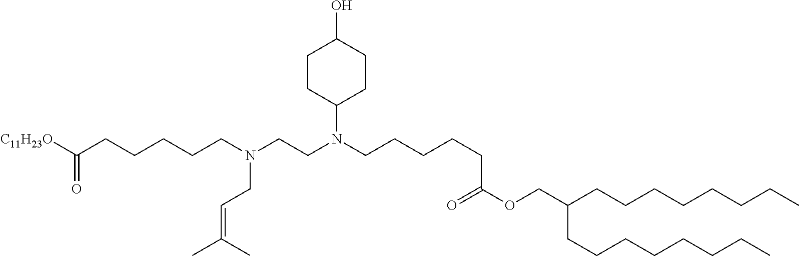

Compound 125

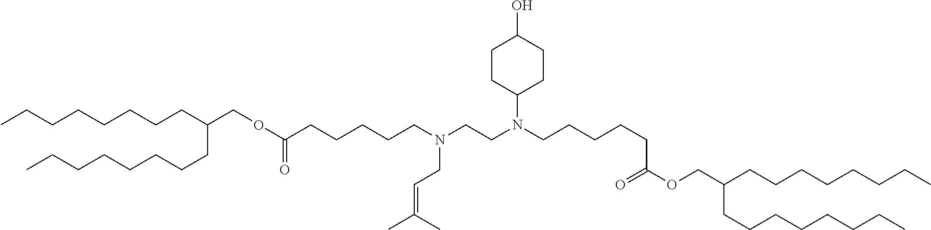 [SMILES: CCCCCCCCC(CCCCCCCC)COC(=O)CCCCCN(CC=C(C)C)CCN(CCCCCC(=O)OCC(CCCCCCCC)CCCCCCCC)C1CCC(O)CC1]

Compound 126

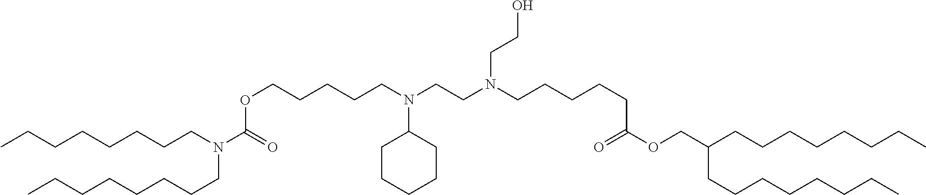 [SMILES: CCCCCCCCC(CCCCCCCC)COC(=O)CCCCCN(CCO)CCN(CCCCCOC(=O)N(CCCCCCCC)CCCCCCCC)C1CCCCC1]

Compound 127

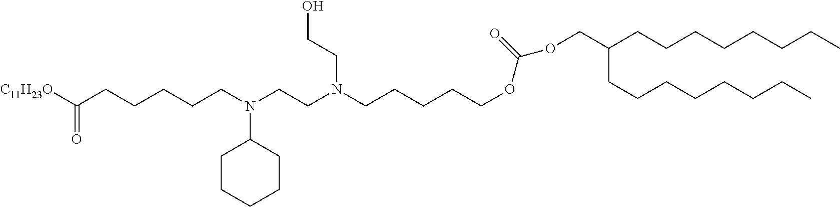

Compound 128

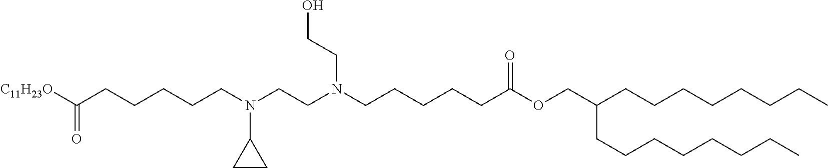

Compound 129

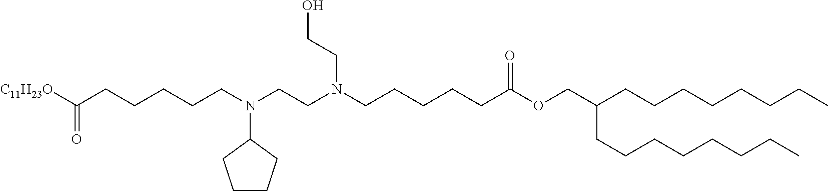

Compound 130

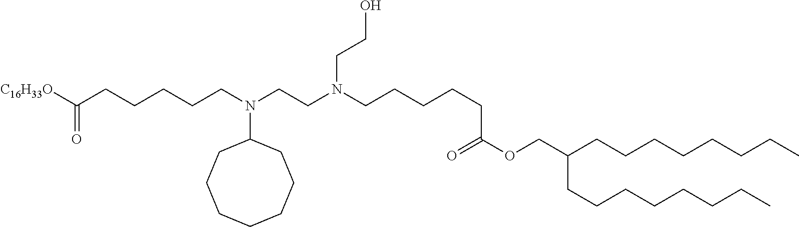

Compound 131

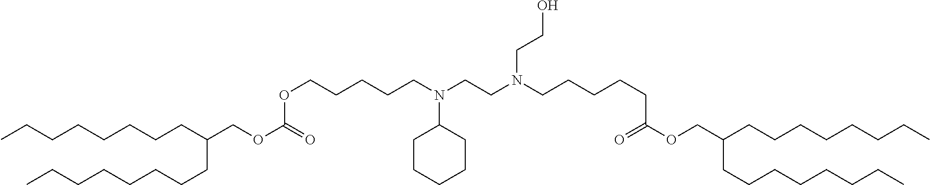 [SMILES: CCCCCCCCC(CCCCCCCC)COC(=O)CCCCCN(CCO)CCN(CCCCCOC(=O)OCC(CCCCCCCC)CCCCCCCC)C1CCCCC1]

Compound 133

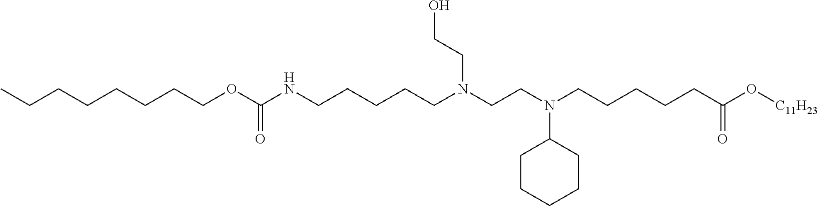

Compound 134

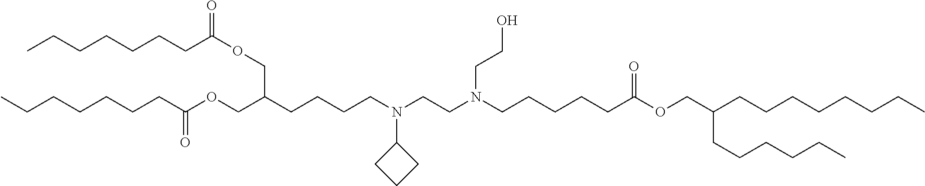 [SMILES: CCCCCCCCC(CCCCCC)COC(=O)CCCCCN(CCO)CCN(CCCCC(COC(=O)CCCCCCC)COC(=O)CCCCCCC)C1CCC1]

Compound 135

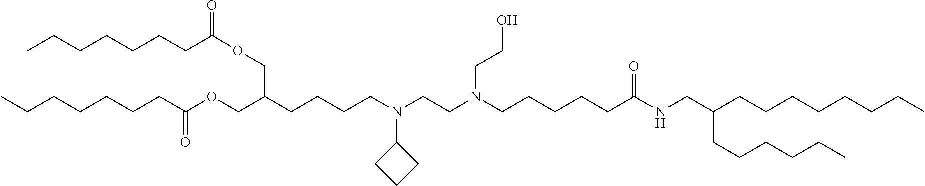 [SMILES: CCCCCCCCC(CCCCCC)CNC(=O)CCCCCN(CCO)CCN(CCCCC(COC(=O)CCCCCCC)COC(=O)CCCCCCC)C1CCC1]

Compound 136

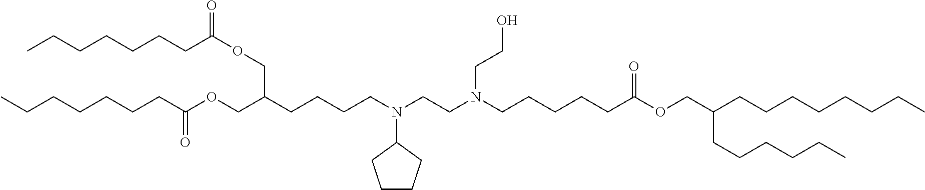 [SMILES: CCCCCCCCC(CCCCCC)COC(=O)CCCCCN(CCO)CCN(CCCCC(COC(=O)CCCCCCC)COC(=O)CCCCCCC)C1CCCC1]

Compound 137

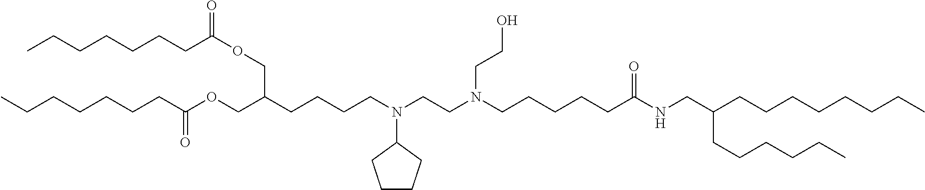 [SMILES: CCCCCCCCC(CCCCCC)CNC(=O)CCCCCN(CCO)CCN(CCCCC(COC(=O)CCCCCCC)COC(=O)CCCCCCC)C1CCCC1]

Compound 138

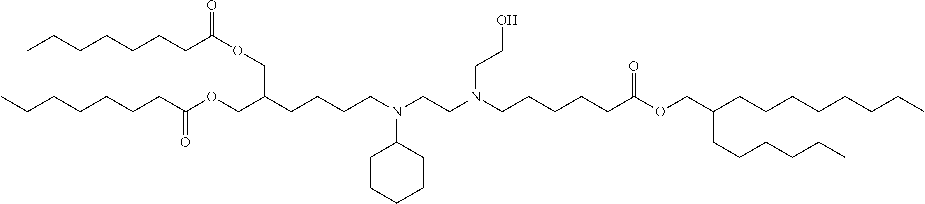 [SMILES: CCCCCCCCC(CCCCCC)COC(=O)CCCCCN(CCO)CCN(CCCCC(COC(=O)CCCCCCC)COC(=O)CCCCCCC)C1CCCCC1]

Compound 139

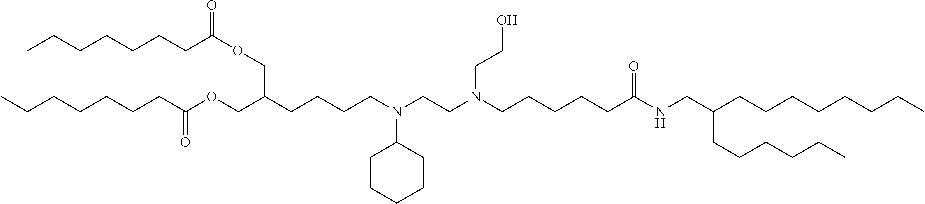 [SMILES: CCCCCCCCC(CCCCCC)CNC(=O)CCCCCN(CCO)CCN(CCCCC(COC(=O)CCCCCCC)COC(=O)CCCCCCC)C1CCCCC1]

Compound 140

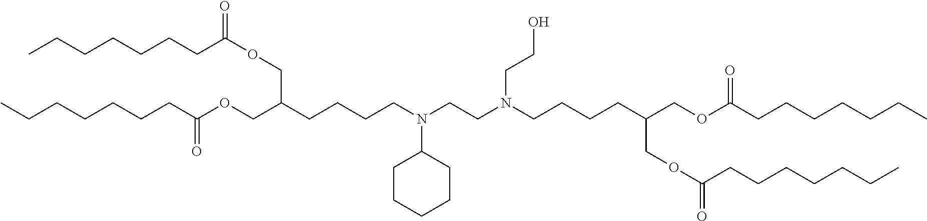 [SMILES: CCCCCCCC(=O)OCC(CCCCN(CCO)CCN(CCCCC(COC(=O)CCCCCCC)COC(=O)CCCCCCC)C1CCCCC1)COC(=O)CCCCCCC]

Compound 141

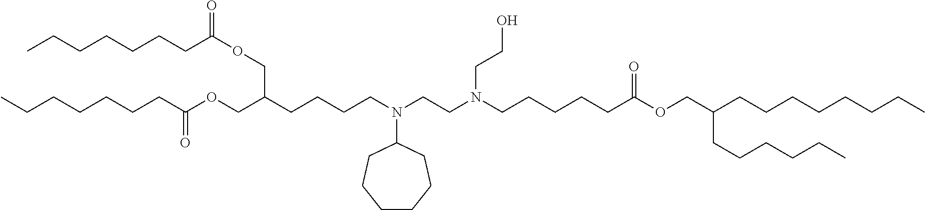 [SMILES: CCCCCCCCC(CCCCCC)COC(=O)CCCCCN(CCO)CCN(CCCCC(COC(=O)CCCCCCC)COC(=O)CCCCCCC)C1CCCCCC1]

Compound 142

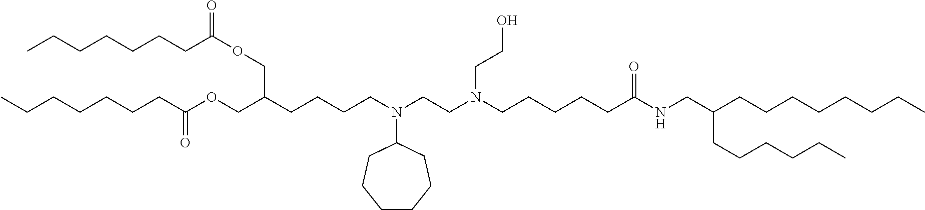 [SMILES: CCCCCCCCC(CCCCCC)CNC(=O)CCCCCN(CCO)CCN(CCCCC(COC(=O)CCCCCCC)COC(=O)CCCCCCC)C1CCCCCC1]

Compound 143

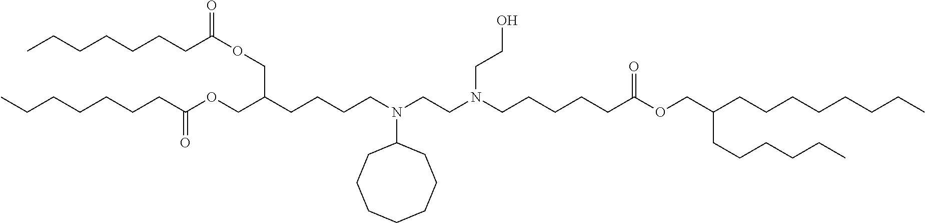 [SMILES: CCCCCCCCC(CCCCCC)COC(=O)CCCCCN(CCO)CCN(CCCCC(COC(=O)CCCCCCC)COC(=O)CCCCCCC)C1CCCCCCC1]

Compound 144

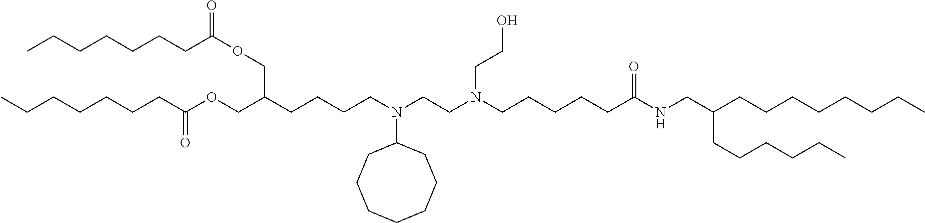 [SMILES: CCCCCCCCC(CCCCCC)CNC(=O)CCCCCN(CCO)CCN(CCCCC(COC(=O)CCCCCCC)COC(=O)CCCCCCC)C1CCCCCCC1]

Compound 145

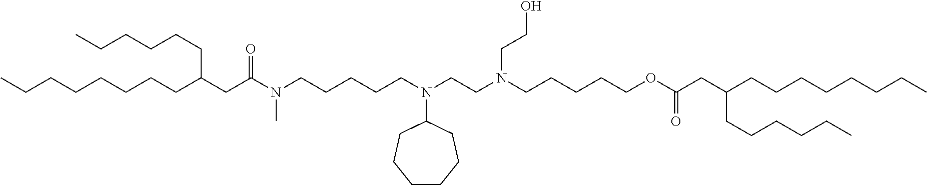 [SMILES: CCCCCCCCC(CCCCCC)CC(=O)OCCCCCN(CCO)CCN(CCCCCN(C)C(=O)CC(CCCCCC)CCCCCCCC)C1CCCCCC1]

Compound 147

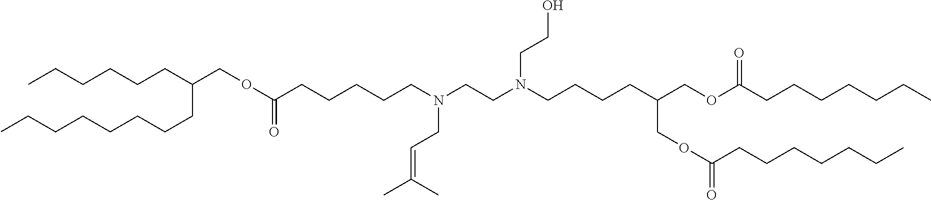 [SMILES: CCCCCCCCC(CCCCCC)COC(=O)CCCCCN(CC=C(C)C)CCN(CCO)CCCCC(COC(=O)CCCCCCC)COC(=O)CCCCCCC]

Compound 148

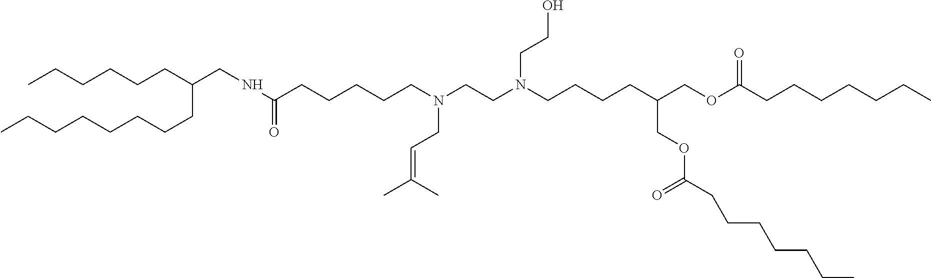 [SMILES: CCCCCCCCC(CCCCCC)CNC(=O)CCCCCN(CC=C(C)C)CCN(CCO)CCCCC(COC(=O)CCCCCCC)COC(=O)CCCCCCC]

Compound 149

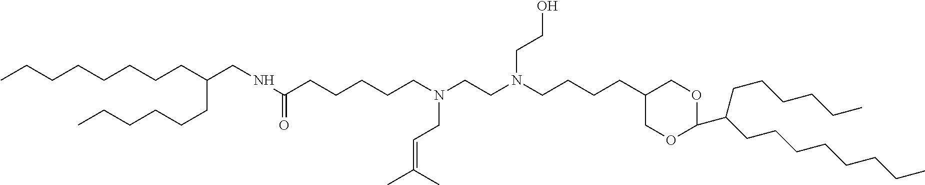 [SMILES: CCCCCCCCC(CCCCCC)CNC(=O)CCCCCN(CC=C(C)C)CCN(CCO)CCCCC1COC(C(CCCCCC)CCCCCCCC)OC1]

Compound 150

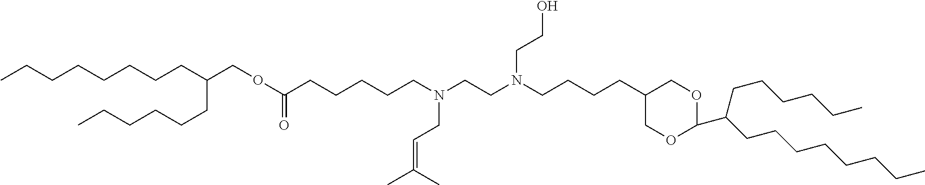 [SMILES: CCCCCCCCC(CCCCCC)COC(=O)CCCCCN(CC=C(C)C)CCN(CCO)CCCCC1COC(C(CCCCCC)CCCCCCCC)OC1]

Compound 151

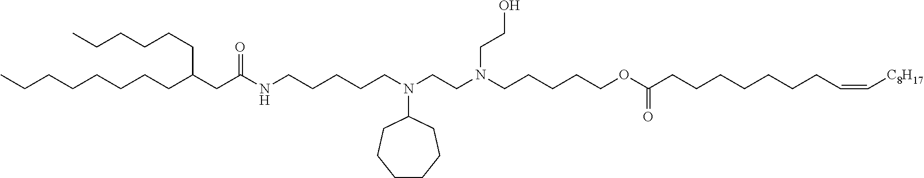

Compound 152

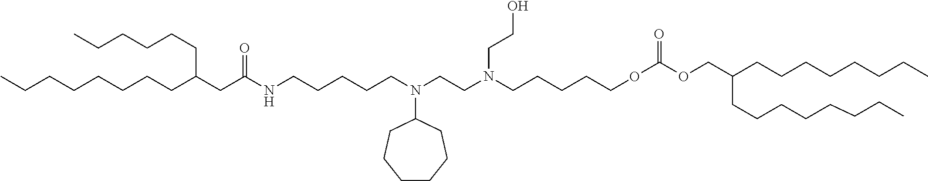 [SMILES: CCCCCCCCC(CCCCCCCC)COC(=O)OCCCCCN(CCO)CCN(CCCCCNC(=O)CC(CCCCCC)CCCCCCCC)C1CCCCCC1]

Compound 153

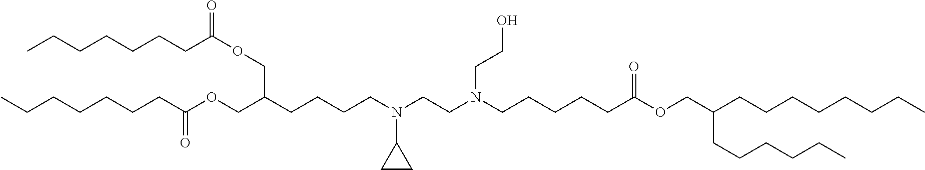 [SMILES: CCCCCCCCC(CCCCCC)COC(=O)CCCCCN(CCO)CCN(CCCCC(COC(=O)CCCCCCC)COC(=O)CCCCCCC)C1CC1]

Compound 154

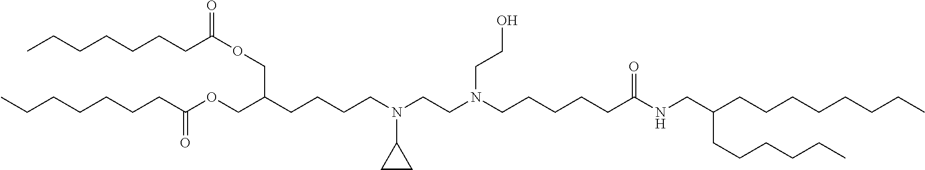 [SMILES: CCCCCCCCC(CCCCCC)CNC(=O)CCCCCN(CCO)CCN(CCCCC(COC(=O)CCCCCCC)COC(=O)CCCCCCC)C1CC1]

Compound 155

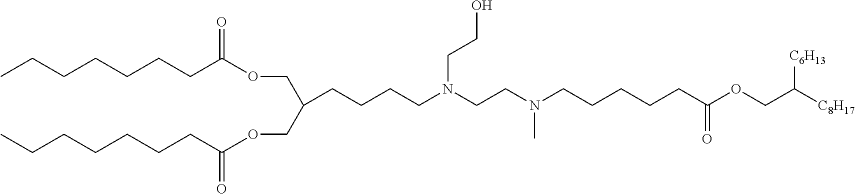

Compound 156

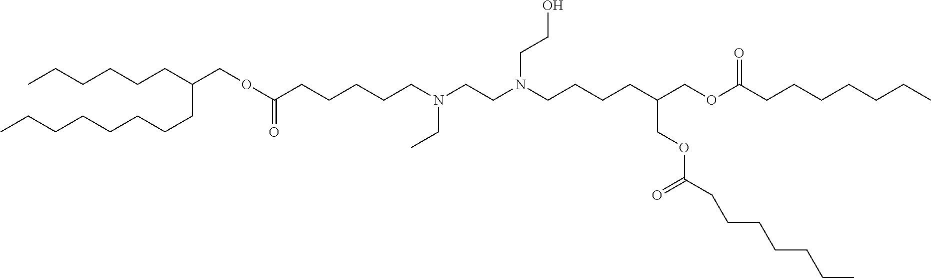 [SMILES: CCCCCCCCC(CCCCCC)COC(=O)CCCCCN(CC)CCN(CCO)CCCCC(COC(=O)CCCCCCC)COC(=O)CCCCCCC]

Compound 157

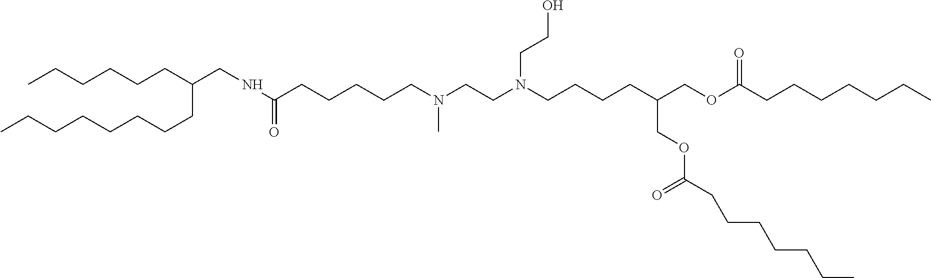 [SMILES: CCCCCCCCC(CCCCCC)CNC(=O)CCCCCN(C)CCN(CCO)CCCCC(COC(=O)CCCCCCC)COC(=O)CCCCCCC]

Compound 158

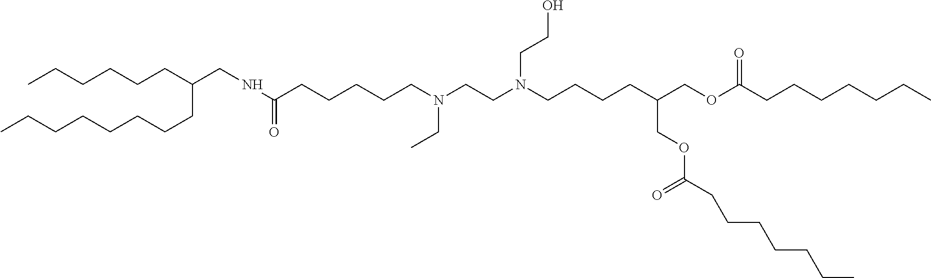 [SMILES: CCCCCCCCC(CCCCCC)CNC(=O)CCCCCN(CC)CCN(CCO)CCCCC(COC(=O)CCCCCCC)COC(=O)CCCCCCC]

Compound 159

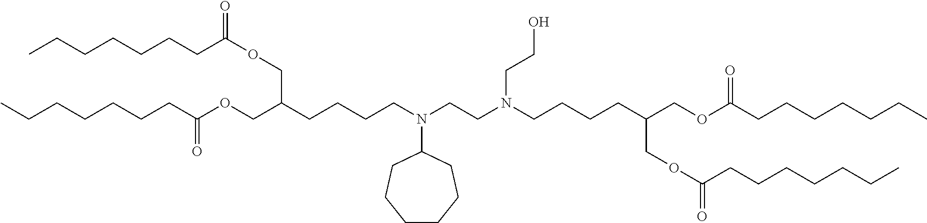 [SMILES: CCCCCCCC(=O)OCC(CCCCN(CCO)CCN(CCCCC(COC(=O)CCCCCCC)COC(=O)CCCCCCC)C1CCCCCC1)COC(=O)CCCCCCC]

Compound 160

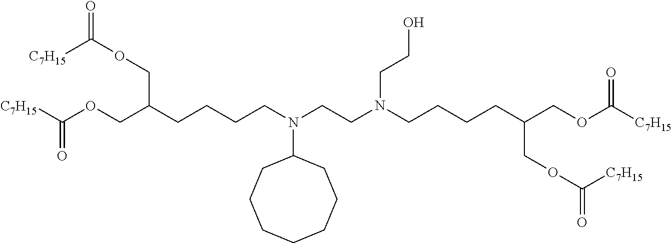

Compound 161

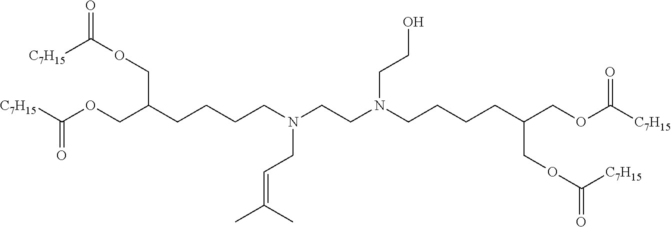

Compound 162

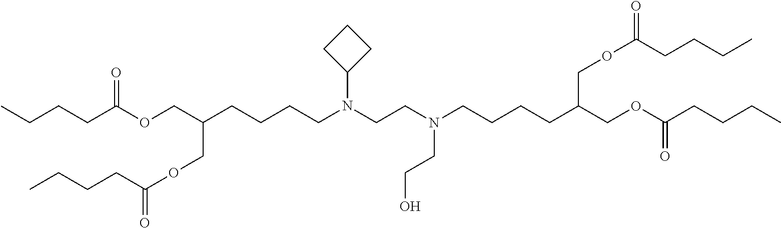 [SMILES: CCCCC(=O)OCC(CCCCN(CCO)CCN(CCCCC(COC(=O)CCCC)COC(=O)CCCC)C1CCC1)COC(=O)CCCC]

Compound 163

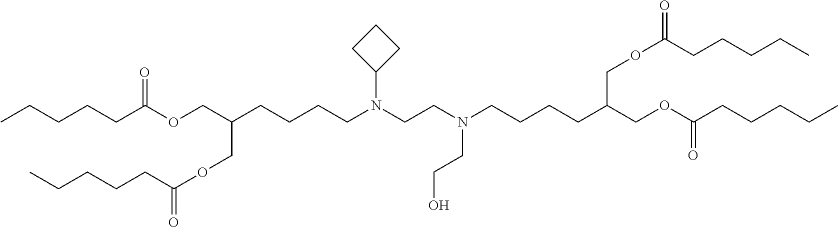 [SMILES: CCCCCC(=O)OCC(CCCCN(CCO)CCN(CCCCC(COC(=O)CCCCC)COC(=O)CCCCC)C1CCC1)COC(=O)CCCCC]

Compound 164

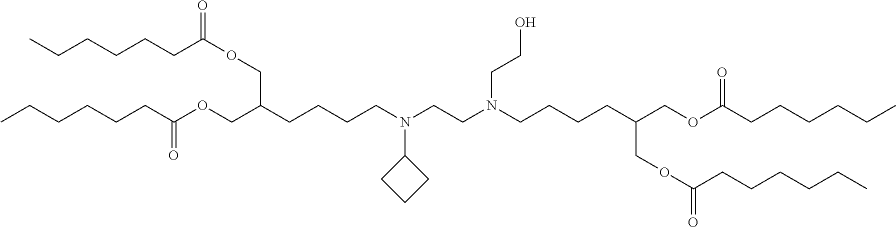 [SMILES: CCCCCCC(=O)OCC(CCCCN(CCO)CCN(CCCCC(COC(=O)CCCCCC)COC(=O)CCCCCC)C1CCC1)COC(=O)CCCCCC]

Compound 165

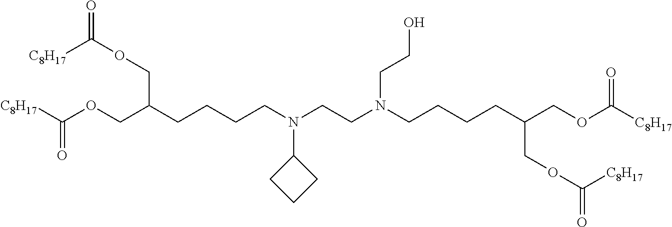

Compound 166

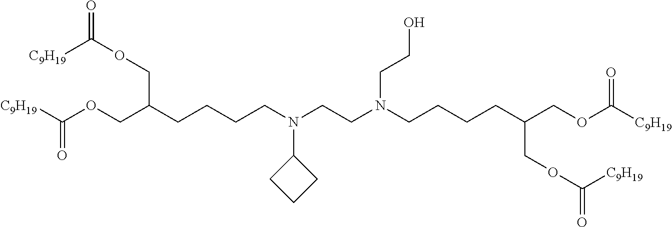

Compound 167

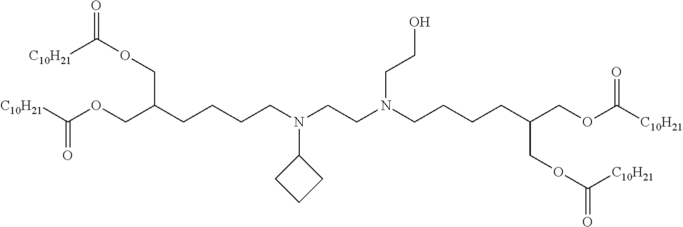

Compound 168

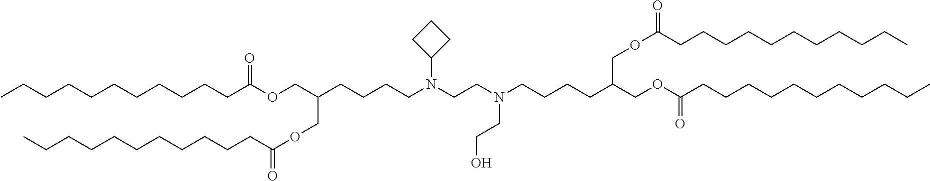 [SMILES: CCCCCCCCCCCC(=O)OCC(CCCCN(CCO)CCN(CCCCC(COC(=O)CCCCCCCCCCC)COC(=O)CCCCCCCCCCC)C1CCC1)COC(=O)CCCCCCCCCCC]

Compound 169

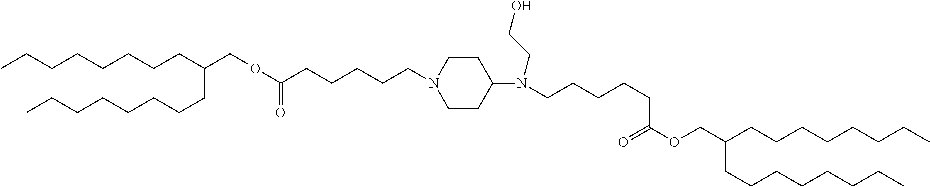 [SMILES: CCCCCCCCC(CCCCCCCC)COC(=O)CCCCCN1CCC(N(CCO)CCCCCC(=O)OCC(CCCCCCCC)CCCCCCCC)CC1]

Compound 170

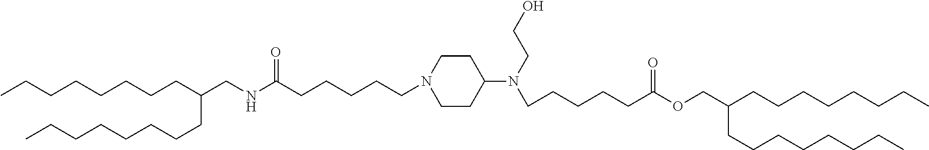 [SMILES: CCCCCCCCC(CCCCCCCC)CNC(=O)CCCCCN1CCC(N(CCO)CCCCCC(=O)OCC(CCCCCCCC)CCCCCCCC)CC1]

Compound 171

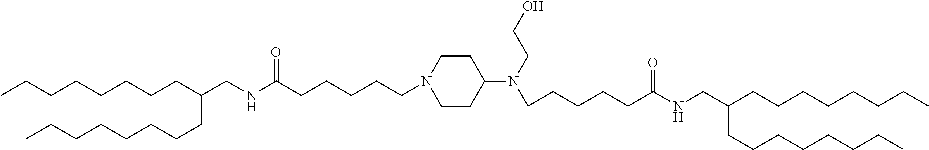 [SMILES: CCCCCCCCC(CCCCCCCC)CNC(=O)CCCCCN1CCC(N(CCO)CCCCCC(=O)NCC(CCCCCCCC)CCCCCCCC)CC1]

Compound 172

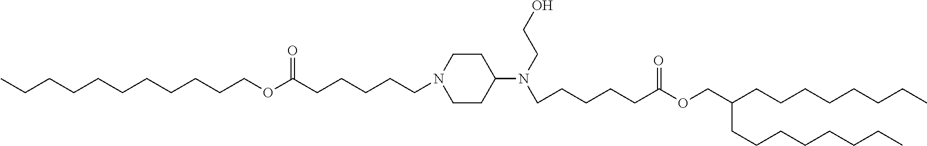 [SMILES: CCCCCCCCCCCOC(=O)CCCCCN1CCC(N(CCO)CCCCCC(=O)OCC(CCCCCCCC)CCCCCCCC)CC1]

Compound 173

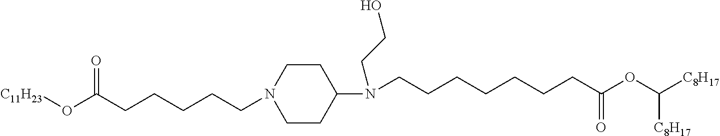

Compound 174

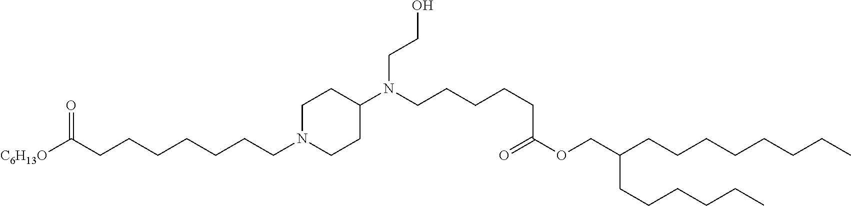

Compound 175

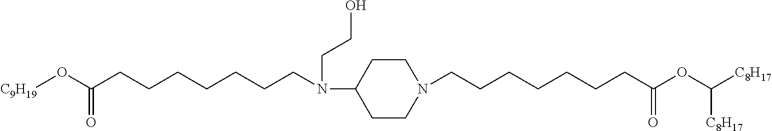

Compound 176

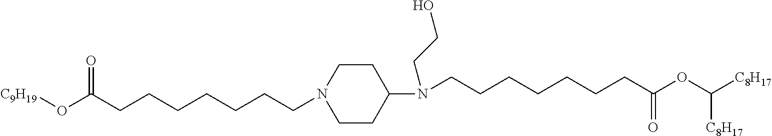

Compound 177

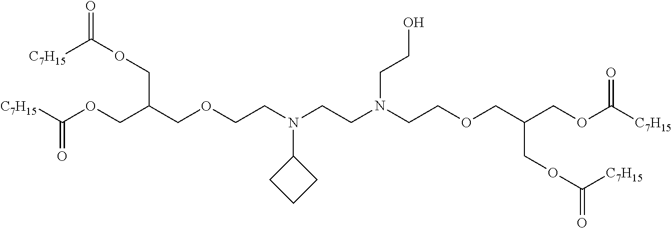

Compound 178

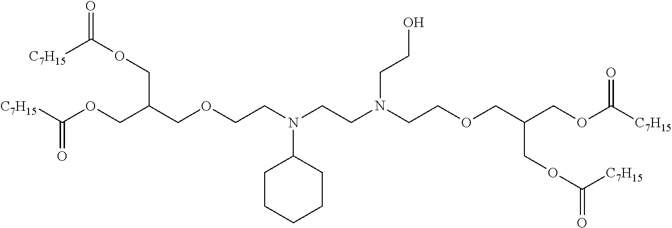

Compound 179

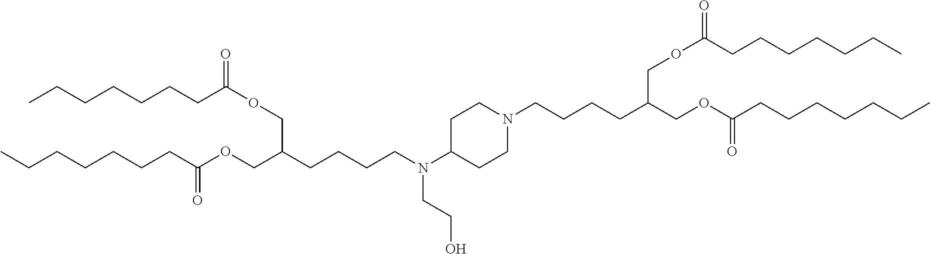 [SMILES: CCCCCCCC(=O)OCC(CCCCN1CCC(N(CCO)CCCCC(COC(=O)CCCCCCC)COC(=O)CCCCCCC)CC1)COC(=O)CCCCCCC]

Compound 180

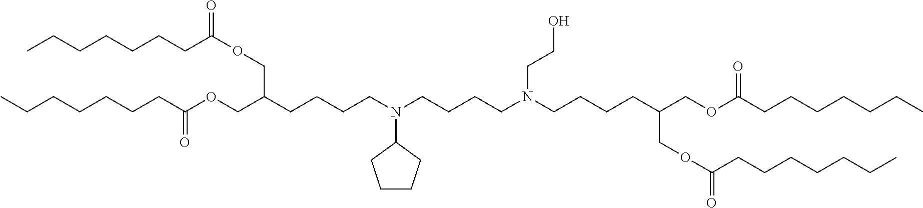 [SMILES: CCCCCCCC(=O)OCC(CCCCN(CCO)CCCCN(CCCCC(COC(=O)CCCCCCC)COC(=O)CCCCCCC)C1CCCC1)COC(=O)CCCCCCC]

Compound 181

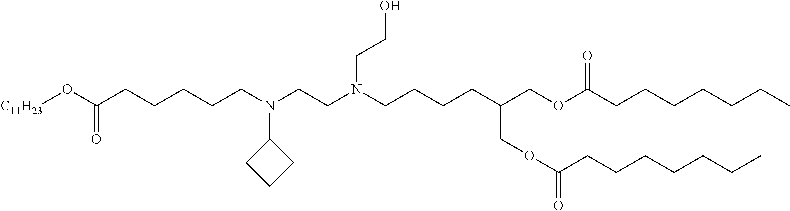

Compound 182

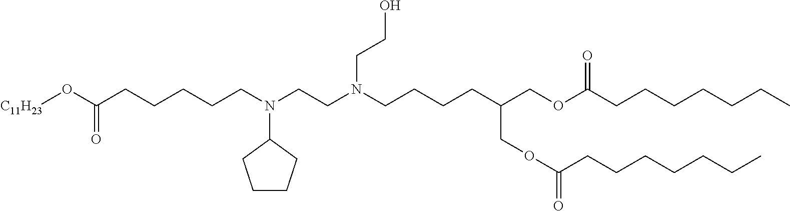

Compound 183

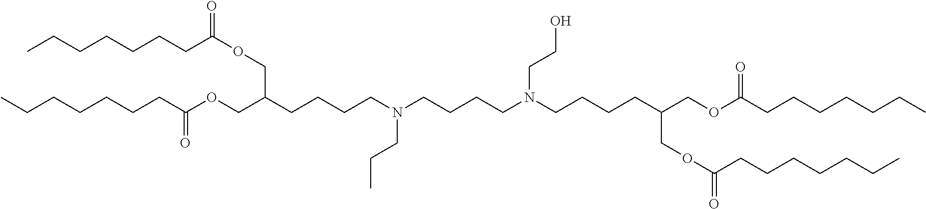 [SMILES: CCCCCCCC(=O)OCC(CCCCN(CCC)CCCCN(CCO)CCCCC(COC(=O)CCCCCCC)COC(=O)CCCCCCC)COC(=O)CCCCCCC]

Compound 184

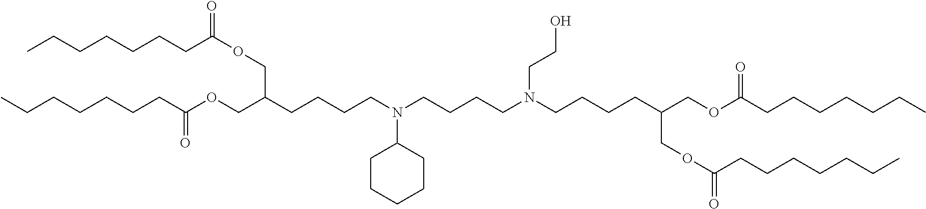 [SMILES: CCCCCCCC(=O)OCC(CCCCN(CCO)CCCCN(CCCCC(COC(=O)CCCCCCC)COC(=O)CCCCCCC)C1CCCCC1)COC(=O)CCCCCCC]

Compound 185

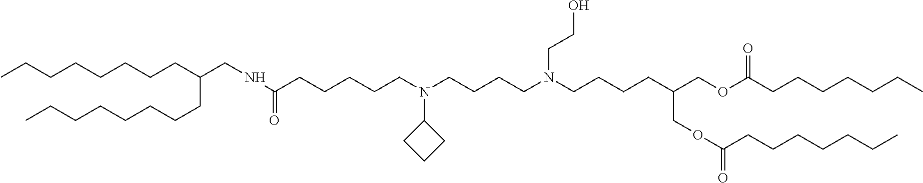 [SMILES: CCCCCCCCC(CCCCCCCC)CNC(=O)CCCCCN(CCCCN(CCO)CCCCC(COC(=O)CCCCCCC)COC(=O)CCCCCCC)C1CCC1]

Compound 186

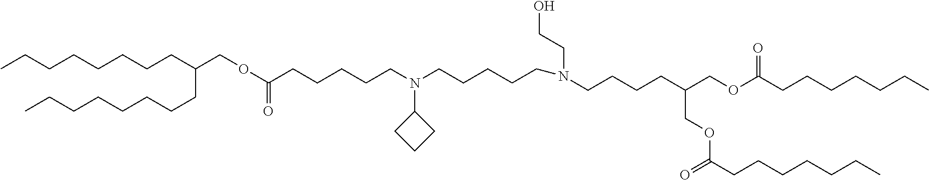 [SMILES: CCCCCCCCC(CCCCCCCC)COC(=O)CCCCCN(CCCCCN(CCO)CCCCC(COC(=O)CCCCCCC)COC(=O)CCCCCCC)C1CCC1]

Compound 187

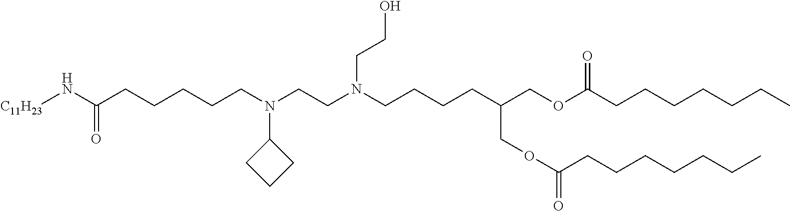

Compound 188

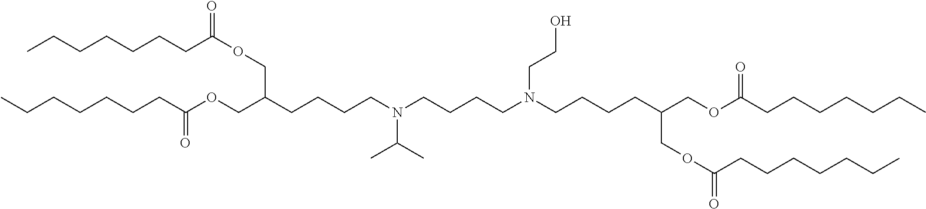 [SMILES: CCCCCCCC(=O)OCC(CCCCN(CCO)CCCCN(CCCCC(COC(=O)CCCCCCC)COC(=O)CCCCCCC)C(C)C)COC(=O)CCCCCCC]

Compound 189

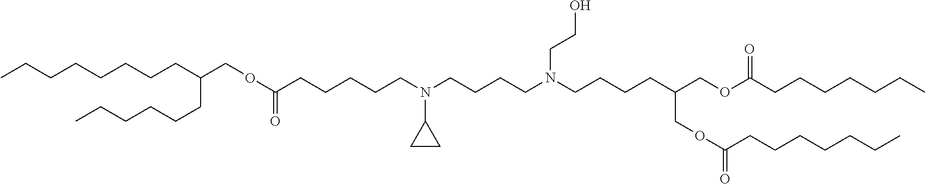 [SMILES: CCCCCCCCC(CCCCCC)COC(=O)CCCCCN(CCCCN(CCO)CCCCC(COC(=O)CCCCCCC)COC(=O)CCCCCCC)C1CC1]

Compound 190

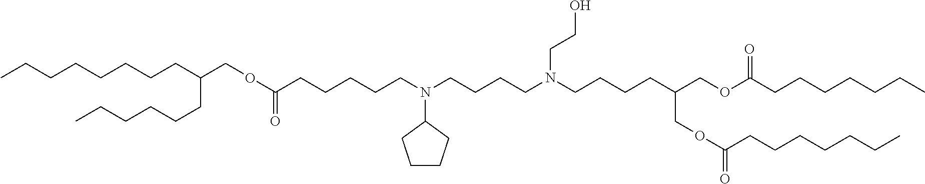 [SMILES: CCCCCCCCC(CCCCCC)COC(=O)CCCCCN(CCCCN(CCO)CCCCC(COC(=O)CCCCCCC)COC(=O)CCCCCCC)C1CCCC1]

Compound 191

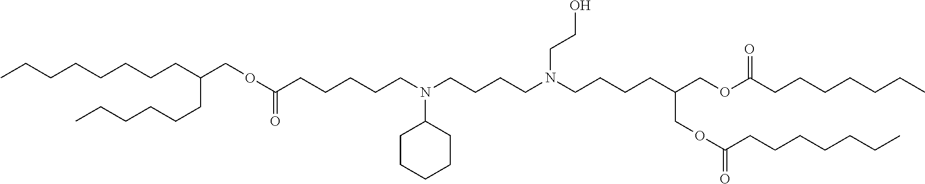 [SMILES: CCCCCCCCC(CCCCCC)COC(=O)CCCCCN(CCCCN(CCO)CCCCC(COC(=O)CCCCCCC)COC(=O)CCCCCCC)C1CCCCC1]

Compound 192

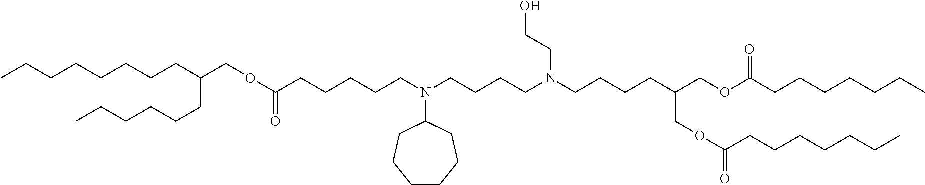 [SMILES: CCCCCCCCC(CCCCCC)COC(=O)CCCCCN(CCCCN(CCO)CCCCC(COC(=O)CCCCCCC)COC(=O)CCCCCCC)C1CCCCCC1]

Compound 193

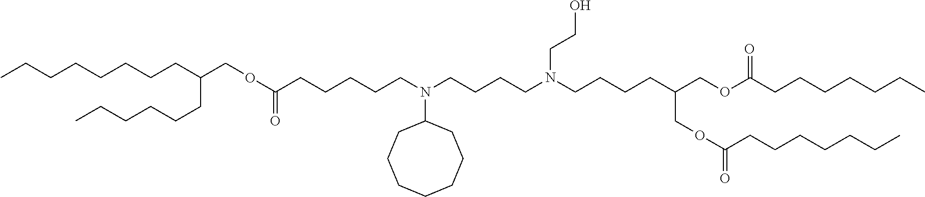 [SMILES: CCCCCCCCC(CCCCCC)COC(=O)CCCCCN(CCCCN(CCO)CCCCC(COC(=O)CCCCCCC)COC(=O)CCCCCCC)C1CCCCCCC1]

Compound 194

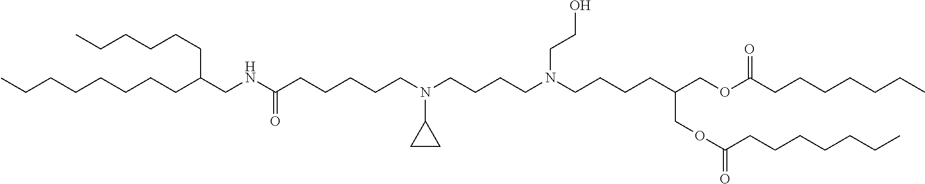 [SMILES: CCCCCCCCC(CCCCCC)CNC(=O)CCCCCN(CCCCN(CCO)CCCCC(COC(=O)CCCCCCC)COC(=O)CCCCCCC)C1CC1]

Compound 195

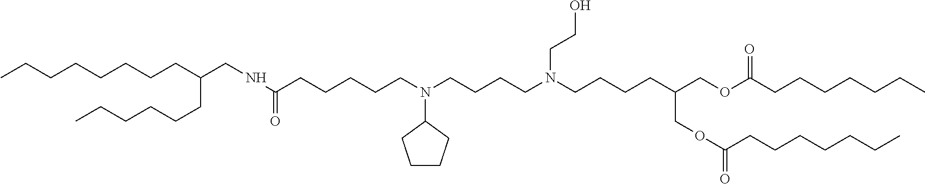 [SMILES: CCCCCCCCC(CCCCCC)CNC(=O)CCCCCN(CCCCN(CCO)CCCCC(COC(=O)CCCCCCC)COC(=O)CCCCCCC)C1CCCC1]

Compound 196

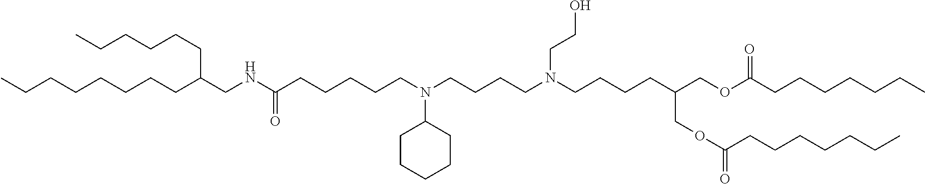 [SMILES: CCCCCCCCC(CCCCCC)CNC(=O)CCCCCN(CCCCN(CCO)CCCCC(COC(=O)CCCCCCC)COC(=O)CCCCCCC)C1CCCCC1]

Compound 197

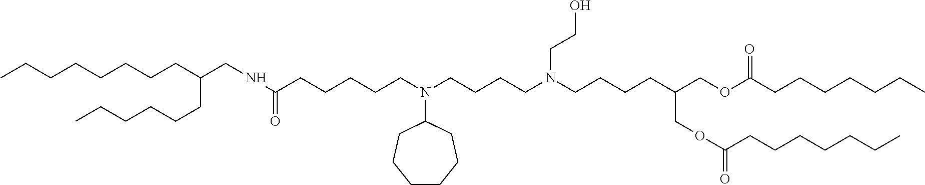 [SMILES: CCCCCCCCC(CCCCCC)CNC(=O)CCCCCN(CCCCN(CCO)CCCCC(COC(=O)CCCCCCC)COC(=O)CCCCCCC)C1CCCCCC1]

Compound 198

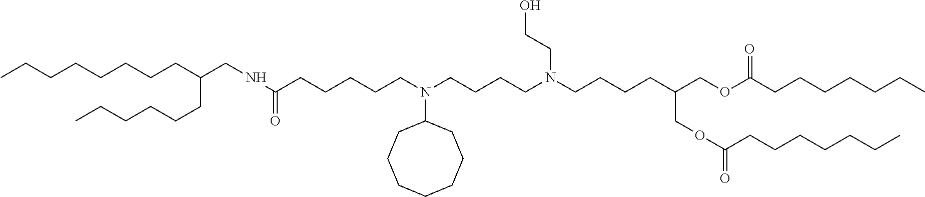 [SMILES: CCCCCCCCC(CCCCCC)CNC(=O)CCCCCN(CCCCN(CCO)CCCCC(COC(=O)CCCCCCC)COC(=O)CCCCCCC)C1CCCCCCC1]

Compound 199

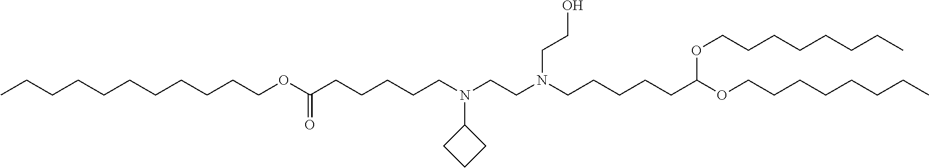 [SMILES: CCCCCCCCCCCOC(=O)CCCCCN(CCN(CCO)CCCCCC(OCCCCCCCC)OCCCCCCCC)C1CCC1]

Compound 200

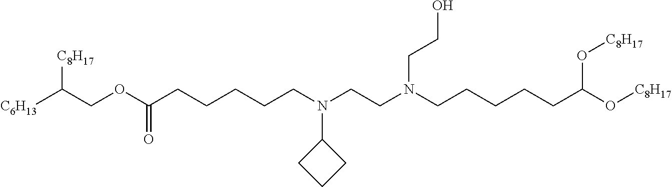

Compound 201

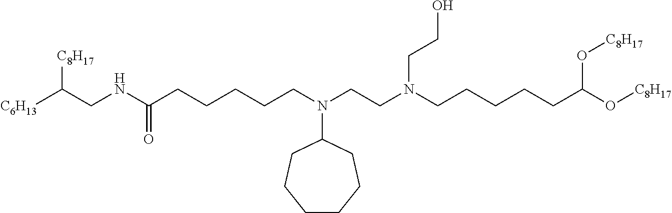

Compound 202

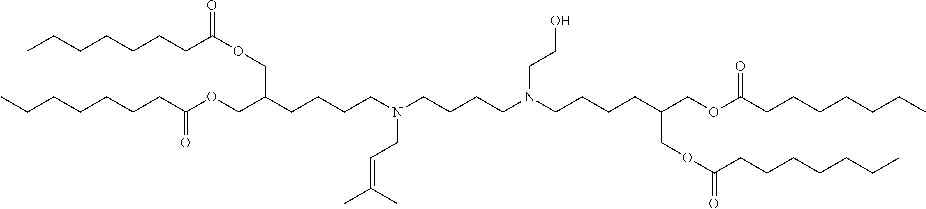 [SMILES: CCCCCCCC(=O)OCC(CCCCN(CC=C(C)C)CCCCN(CCO)CCCCC(COC(=O)CCCCCCC)COC(=O)CCCCCCC)COC(=O)CCCCCCC]

Compound 203

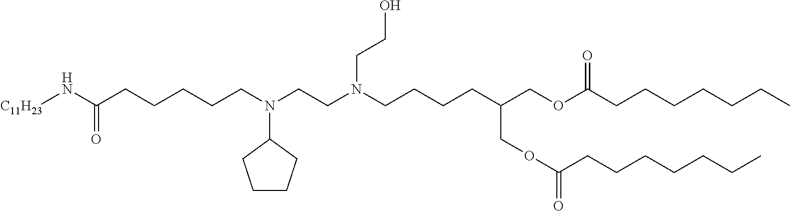

Compound 204

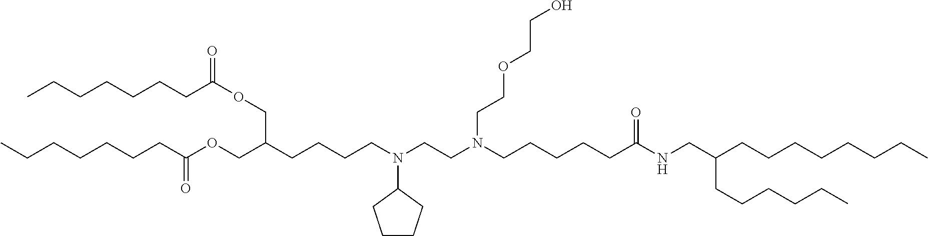 [SMILES: CCCCCCCCC(CCCCCC)CNC(=O)CCCCCN(CCOCCO)CCN(CCCCC(COC(=O)CCCCCCC)COC(=O)CCCCCCC)C1CCCC1]

Compound 205

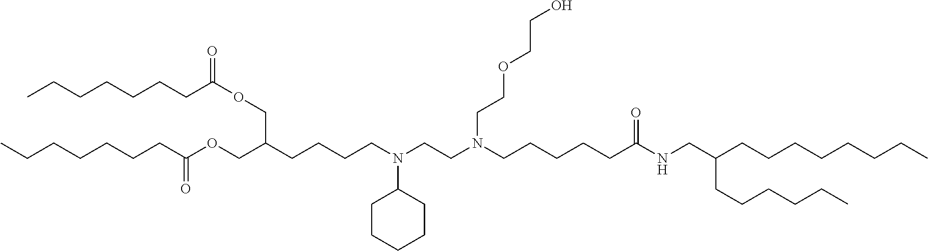 [SMILES: CCCCCCCCC(CCCCCC)CNC(=O)CCCCCN(CCOCCO)CCN(CCCCC(COC(=O)CCCCCCC)COC(=O)CCCCCCC)C1CCCCC1]

Compound 206

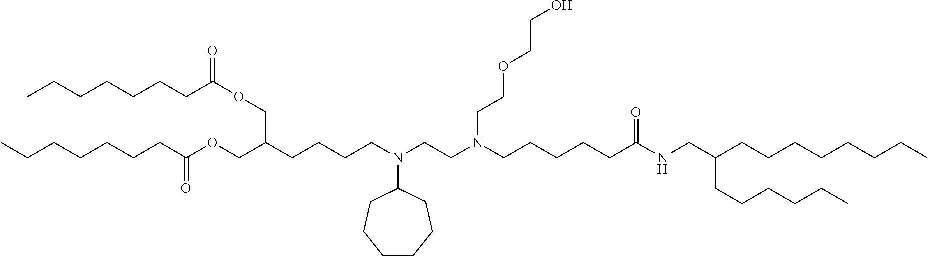 [SMILES: CCCCCCCCC(CCCCCC)CNC(=O)CCCCCN(CCOCCO)CCN(CCCCC(COC(=O)CCCCCCC)COC(=O)CCCCCCC)C1CCCCCC1]

Compound 207

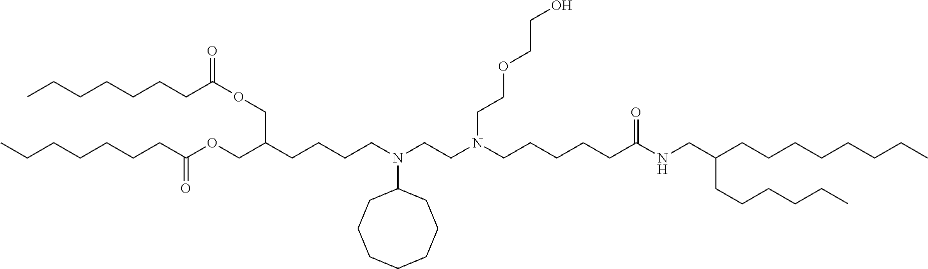 [SMILES: CCCCCCCCC(CCCCCC)CNC(=O)CCCCCN(CCOCCO)CCN(CCCCC(COC(=O)CCCCCCC)COC(=O)CCCCCCC)C1CCCCCCC1]

Compound 208

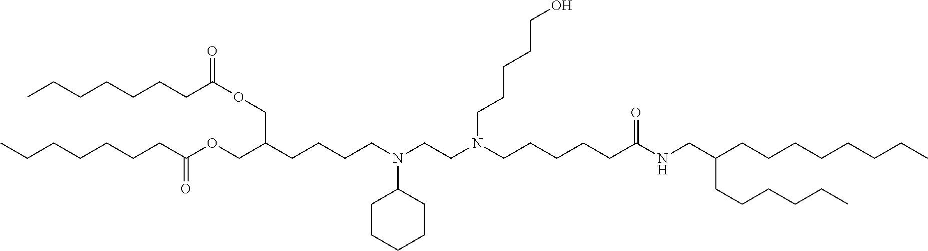 [SMILES: CCCCCCCCC(CCCCCC)CNC(=O)CCCCCN(CCCCCO)CCN(CCCCC(COC(=O)CCCCCCC)COC(=O)CCCCCCC)C1CCCCC1]

Compound 209

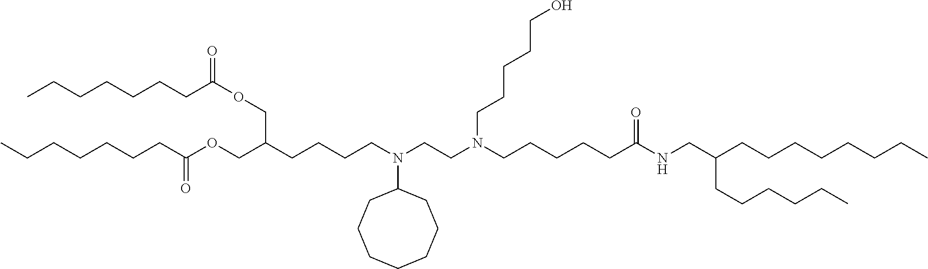 [SMILES: CCCCCCCCC(CCCCCC)CNC(=O)CCCCCN(CCCCCO)CCN(CCCCC(COC(=O)CCCCCCC)COC(=O)CCCCCCC)C1CCCCCCC1]

Compound 210

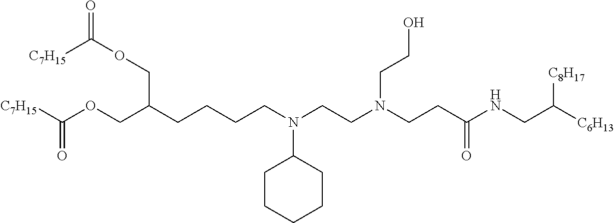

Compound 211

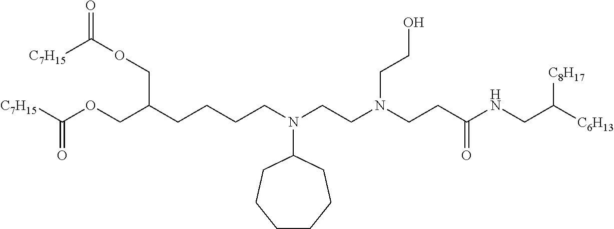

Compound 212

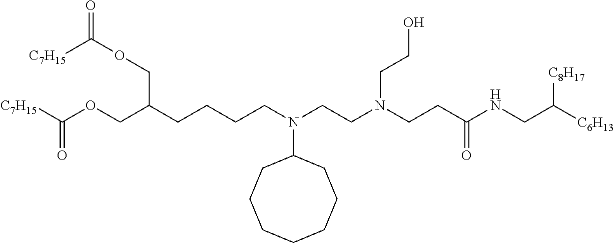

Compound 213

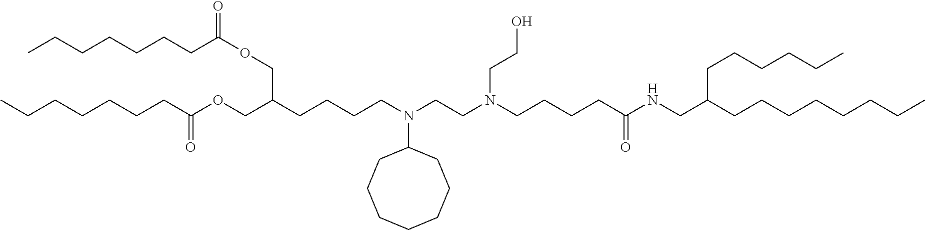 [SMILES: CCCCCCCCC(CCCCCC)CNC(=O)CCCCN(CCO)CCN(CCCCC(COC(=O)CCCCCCC)COC(=O)CCCCCCC)C1CCCCCCC1]

Compound 214

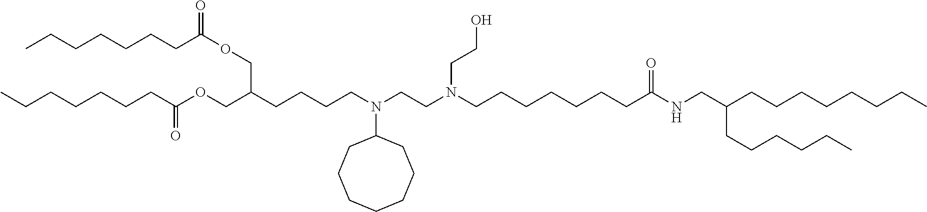 [SMILES: CCCCCCCCC(CCCCCC)CNC(=O)CCCCCCCN(CCO)CCN(CCCCC(COC(=O)CCCCCCC)COC(=O)CCCCCCC)C1CCCCCCC1]

Compound 215

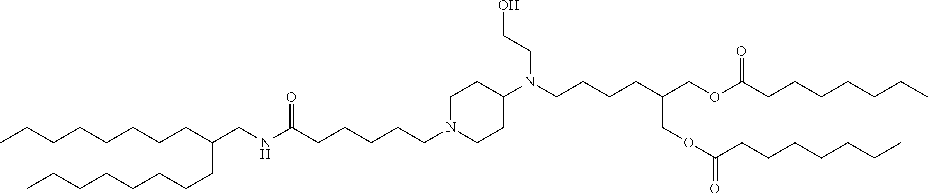 [SMILES: CCCCCCCCC(CCCCCCCC)CNC(=O)CCCCCN1CCC(N(CCO)CCCCC(COC(=O)CCCCCCC)COC(=O)CCCCCCC)CC1]

Compound 218

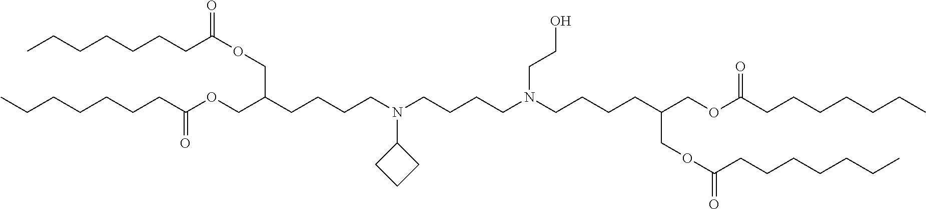 [SMILES: CCCCCCCC(=O)OCC(CCCCN(CCO)CCCCN(CCCCC(COC(=O)CCCCCCC)COC(=O)CCCCCCC)C1CCC1)COC(=O)CCCCCCC]

Compound 219

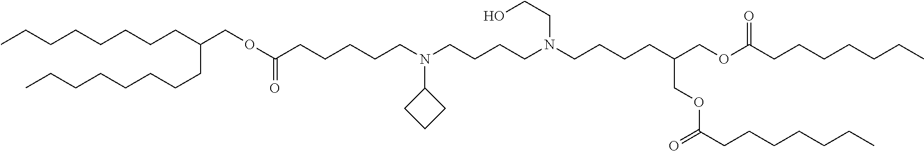 [SMILES: CCCCCCCCC(CCCCCCCC)COC(=O)CCCCCN(CCCCN(CCO)CCCCC(COC(=O)CCCCCCC)COC(=O)CCCCCCC)C1CCC1]

Compound 220

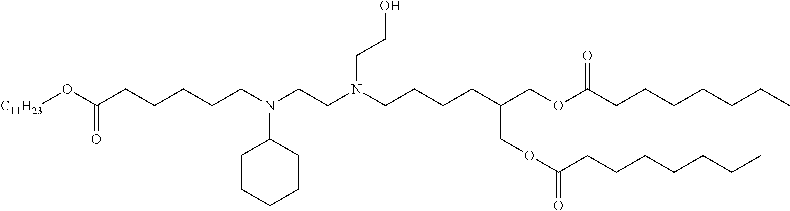

Compound 221

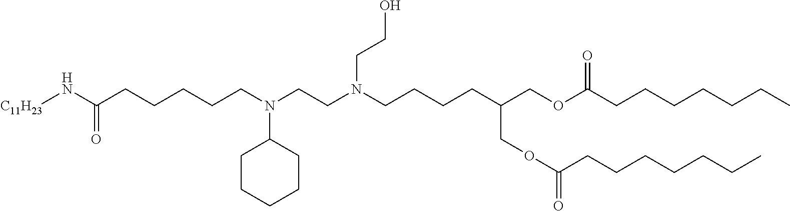

Compound 222

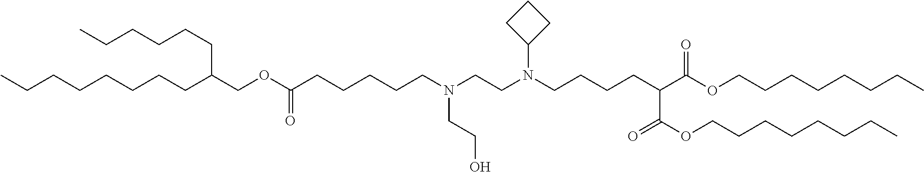 [SMILES: CCCCCCCCOC(=O)C(CCCCN(CCN(CCO)CCCCCC(=O)OCC(CCCCCC)CCCCCCCC)C1CCC1)C(=O)OCCCCCCCC]

Compound 223

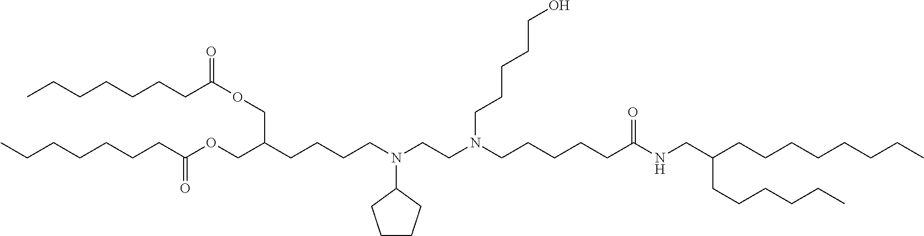 [SMILES: CCCCCCCCC(CCCCCC)CNC(=O)CCCCCN(CCCCCO)CCN(CCCCC(COC(=O)CCCCCCC)COC(=O)CCCCCCC)C1CCCC1]

Compound 224

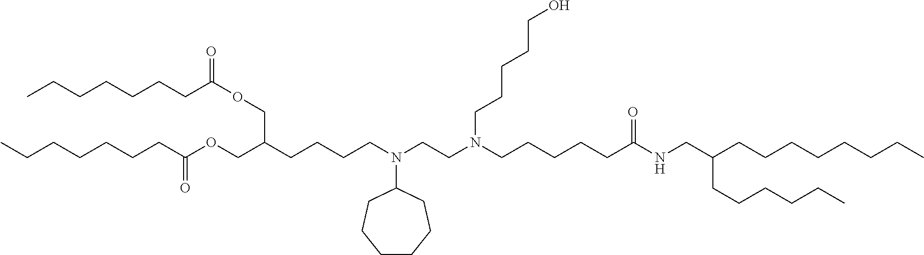 [SMILES: CCCCCCCCC(CCCCCC)CNC(=O)CCCCCN(CCCCCO)CCN(CCCCC(COC(=O)CCCCCCC)COC(=O)CCCCCCC)C1CCCCCC1]

Compound 225

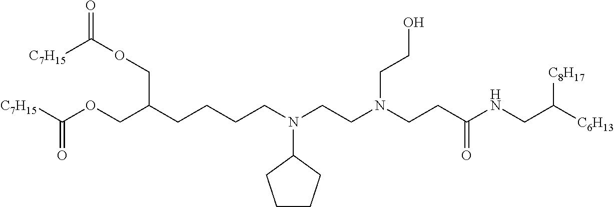

Compound 226

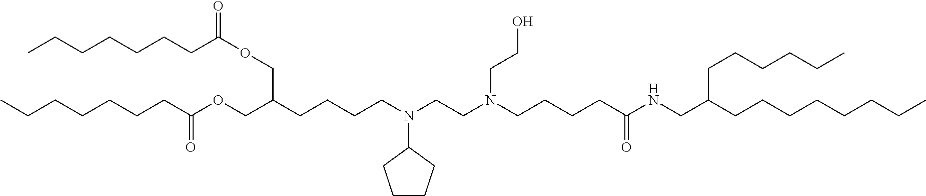 [SMILES: CCCCCCCCC(CCCCCC)CNC(=O)CCCCN(CCO)CCN(CCCCC(COC(=O)CCCCCCC)COC(=O)CCCCCCC)C1CCCC1]

Compound 227

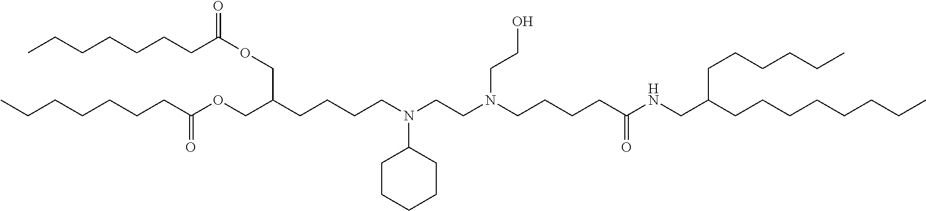 [SMILES: CCCCCCCCC(CCCCCC)CNC(=O)CCCCN(CCO)CCN(CCCCC(COC(=O)CCCCCCC)COC(=O)CCCCCCC)C1CCCCC1]

Compound 228

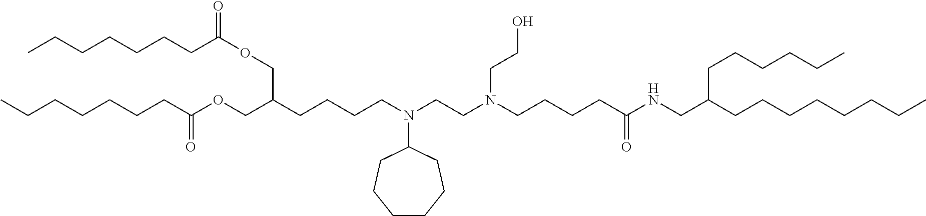 [SMILES: CCCCCCCCC(CCCCCC)CNC(=O)CCCCN(CCO)CCN(CCCCC(COC(=O)CCCCCCC)COC(=O)CCCCCCC)C1CCCCCC1]

Compound 229

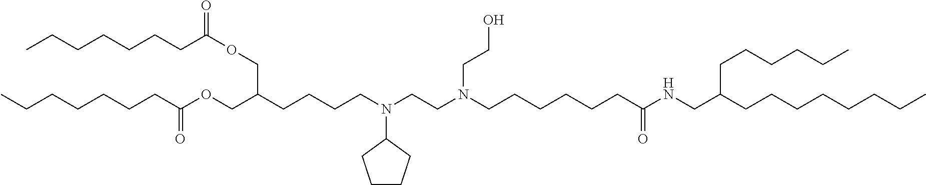 [SMILES: CCCCCCCCC(CCCCCC)CNC(=O)CCCCCCN(CCO)CCN(CCCCC(COC(=O)CCCCCCC)COC(=O)CCCCCCC)C1CCCC1]

Compound 230

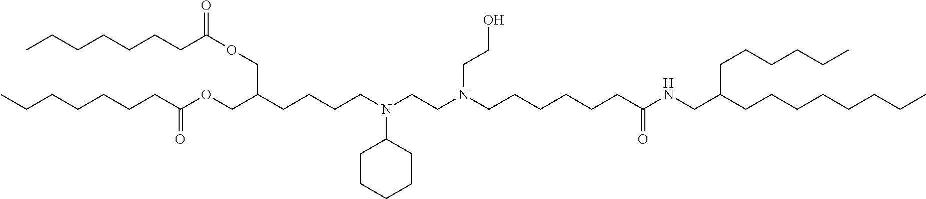 [SMILES: CCCCCCCCC(CCCCCC)CNC(=O)CCCCCCN(CCO)CCN(CCCCC(COC(=O)CCCCCCC)COC(=O)CCCCCCC)C1CCCCC1]

Compound 231

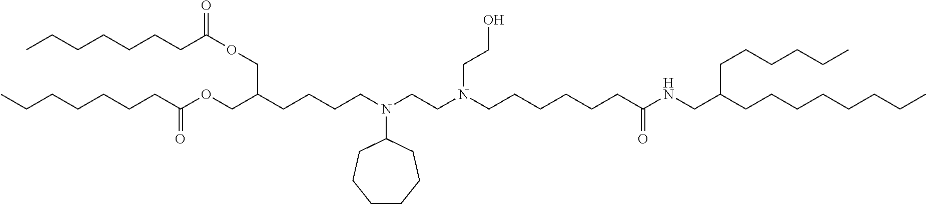 [SMILES: CCCCCCCCC(CCCCCC)CNC(=O)CCCCCCN(CCO)CCN(CCCCC(COC(=O)CCCCCCC)COC(=O)CCCCCCC)C1CCCCCC1]

Compound 232

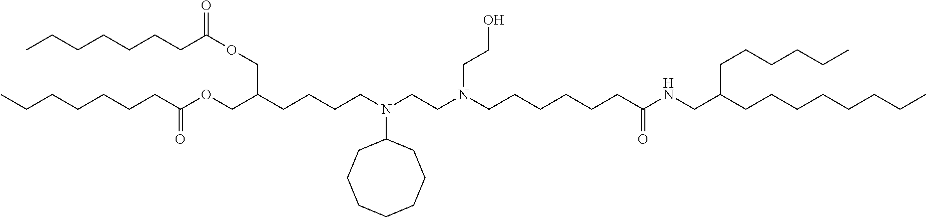 [SMILES: CCCCCCCCC(CCCCCC)CNC(=O)CCCCCCN(CCO)CCN(CCCCC(COC(=O)CCCCCCC)COC(=O)CCCCCCC)C1CCCCCCC1]

Compound 233

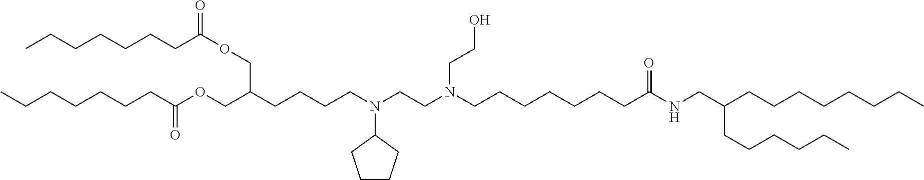 [SMILES: CCCCCCCCC(CCCCCC)CNC(=O)CCCCCCCN(CCO)CCN(CCCCC(COC(=O)CCCCCCC)COC(=O)CCCCCCC)C1CCCC1]

Compound 234

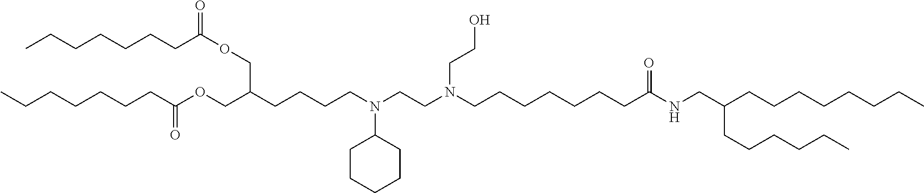 [SMILES: CCCCCCCCC(CCCCCC)CNC(=O)CCCCCCCN(CCO)CCN(CCCCC(COC(=O)CCCCCCC)COC(=O)CCCCCCC)C1CCCCC1]

Compound 235

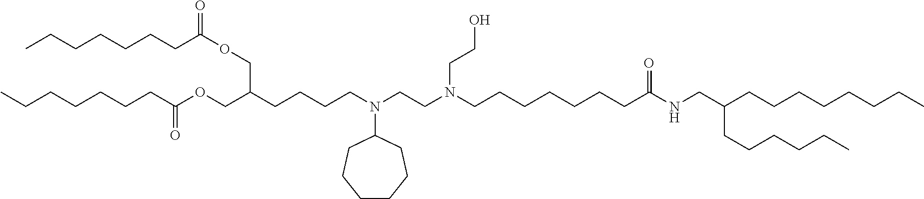 [SMILES: CCCCCCCCC(CCCCCC)CNC(=O)CCCCCCCN(CCO)CCN(CCCCC(COC(=O)CCCCCCC)COC(=O)CCCCCCC)C1CCCCCC1]

Compound 236

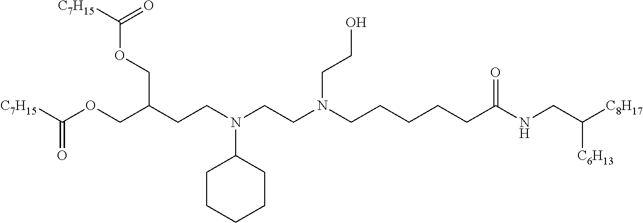

Compound 237

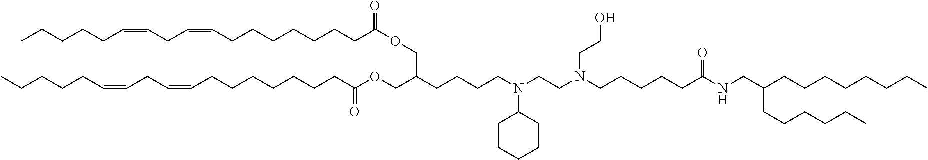 [SMILES: CCCCC/C=C\C/C=C\CCCCCCCC(=O)OCC(CCCCN(CCN(CCO)CCCCCC(=O)NCC(CCCCCC)CCCCCCCC)C1CCCCC1)COC(=O)CCCCCCC/C=C\C/C=C\CCCCC]

Compound 238

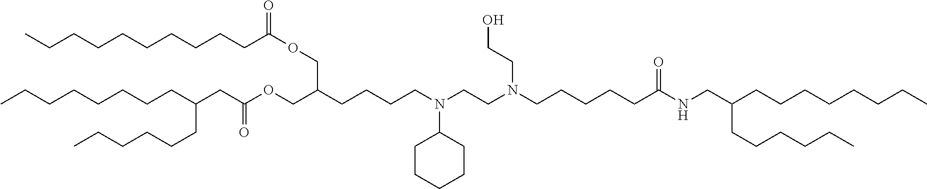 [SMILES: CCCCCCCCCCC(=O)OCC(CCCCN(CCN(CCO)CCCCCC(=O)NCC(CCCCCC)CCCCCCCC)C1CCCCC1)COC(=O)CC(CCCCCC)CCCCCCCC]

Compound 239

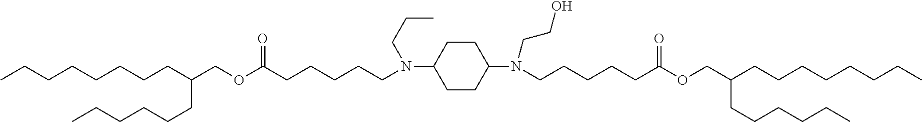 [SMILES: CCCCCCCCC(CCCCCC)COC(=O)CCCCCN(CCC)C1CCC(N(CCO)CCCCCC(=O)OCC(CCCCCC)CCCCCCCC)CC1]

Compound 241

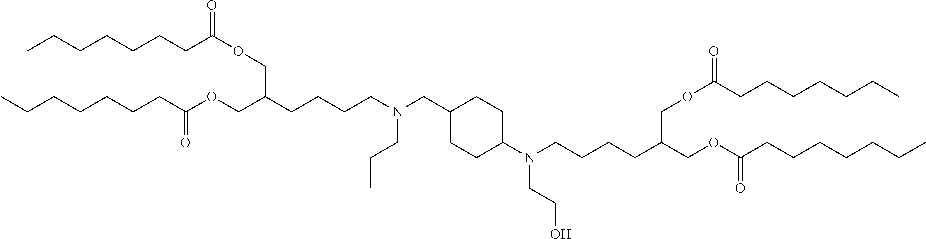 [SMILES: CCCCCCCC(=O)OCC(CCCCN(CCC)CC1CCC(N(CCO)CCCCC(COC(=O)CCCCCCC)COC(=O)CCCCCCC)CC1)COC(=O)CCCCCCC]

Compound 242

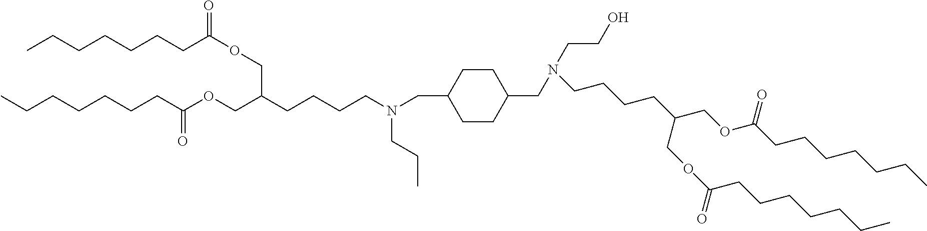 [SMILES: CCCCCCCC(=O)OCC(CCCCN(CCC)CC1CCC(CN(CCO)CCCCC(COC(=O)CCCCCCC)COC(=O)CCCCCCC)CC1)COC(=O)CCCCCCC]

Compound 243

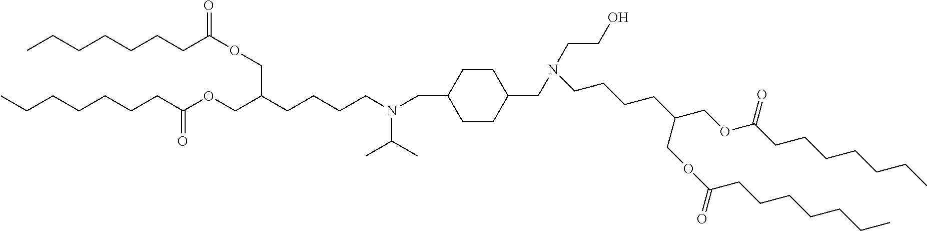 [SMILES: CCCCCCCC(=O)OCC(CCCCN(CCO)CC1CCC(CN(CCCCC(COC(=O)CCCCCCC)COC(=O)CCCCCCC)C(C)C)CC1)COC(=O)CCCCCCC]

Compound 244

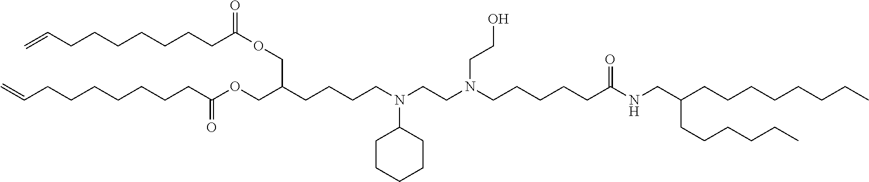 [SMILES: C=CCCCCCCCC(=O)OCC(CCCCN(CCN(CCO)CCCCCC(=O)NCC(CCCCCC)CCCCCCCC)C1CCCCC1)COC(=O)CCCCCCCC=C]

Compound 245

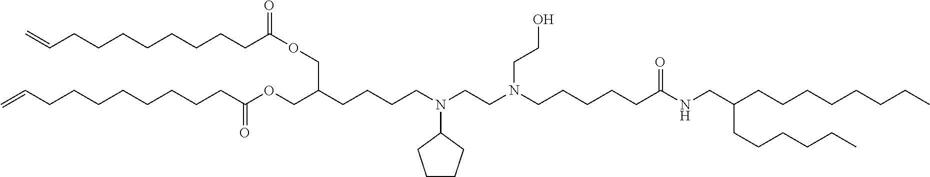 [SMILES: C=CCCCCCCCCC(=O)OCC(CCCCN(CCN(CCO)CCCCCC(=O)NCC(CCCCCC)CCCCCCCC)C1CCCC1)COC(=O)CCCCCCCCC=C]

Compound 246

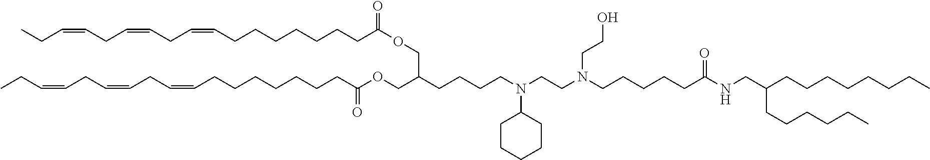 [SMILES: CC/C=C\C/C=C\C/C=C\CCCCCCCC(=O)OCC(CCCCN(CCN(CCO)CCCCCC(=O)NCC(CCCCCC)CCCCCCCC)C1CCCCC1)COC(=O)CCCCCCC/C=C\C/C=C\C/C=C\CC]

Compound 247

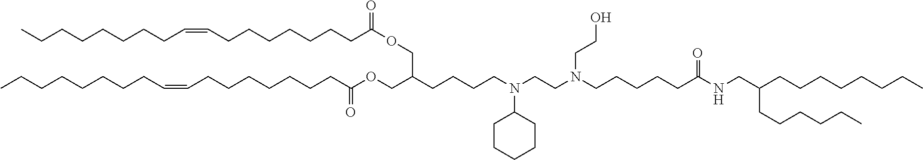 [SMILES: CCCCCCCC/C=C\CCCCCCCC(=O)OCC(CCCCN(CCN(CCO)CCCCCC(=O)NCC(CCCCCC)CCCCCCCC)C1CCCCC1)COC(=O)CCCCCCC/C=C\CCCCCCCC]

Compound 248

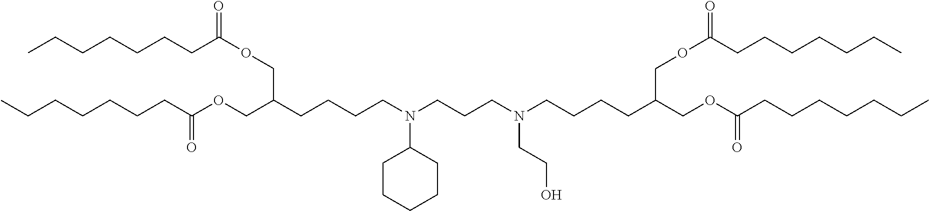 [SMILES: CCCCCCCC(=O)OCC(CCCCN(CCO)CCCN(CCCCC(COC(=O)CCCCCCC)COC(=O)CCCCCCC)C1CCCCC1)COC(=O)CCCCCCC]

Compound 249

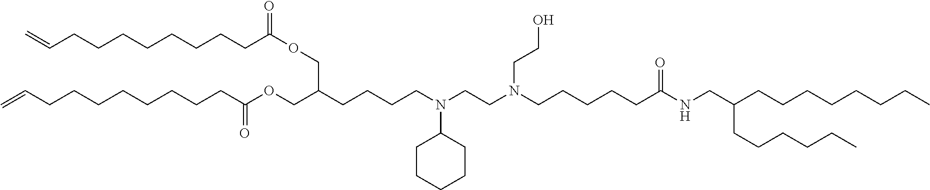 [SMILES: C=CCCCCCCCCC(=O)OCC(CCCCN(CCN(CCO)CCCCCC(=O)NCC(CCCCCC)CCCCCCCC)C1CCCCC1)COC(=O)CCCCCCCCC=C]

Compound 250

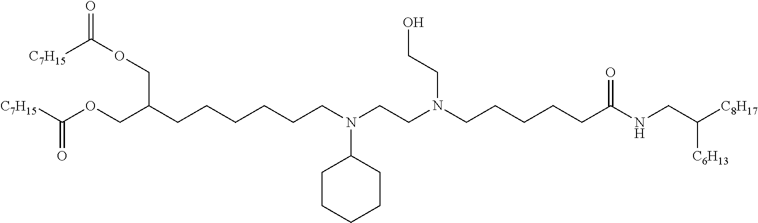

Compound 251

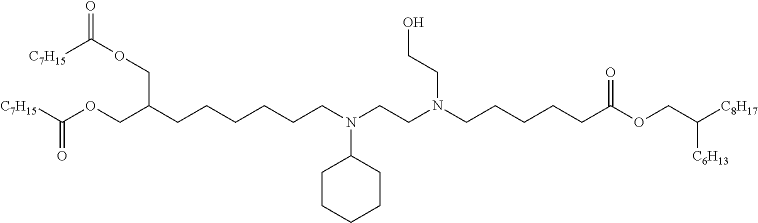

Compound 252

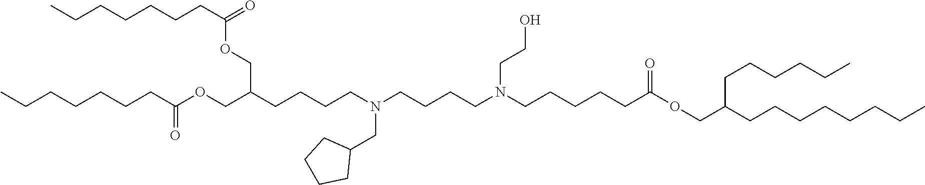 [SMILES: CCCCCCCCC(CCCCCC)COC(=O)CCCCCN(CCO)CCCCN(CCCCC(COC(=O)CCCCCCC)COC(=O)CCCCCCC)CC1CCCC1]

Compound 253

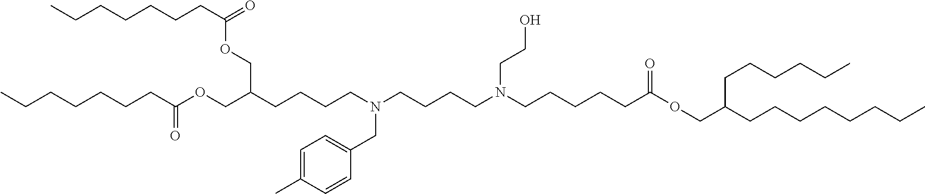 [SMILES: CCCCCCCCC(CCCCCC)COC(=O)CCCCCN(CCO)CCCCN(CCCCC(COC(=O)CCCCCCC)COC(=O)CCCCCCC)Cc1ccc(C)cc1]

Compound 254

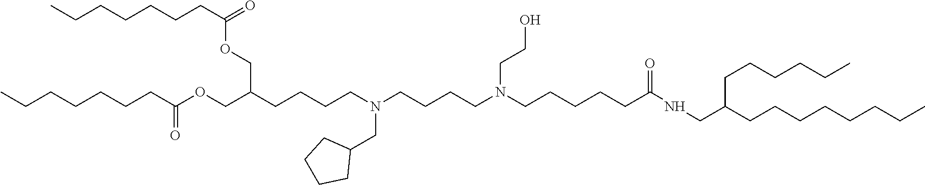 [SMILES: CCCCCCCCC(CCCCCC)CNC(=O)CCCCCN(CCO)CCCCN(CCCCC(COC(=O)CCCCCCC)COC(=O)CCCCCCC)CC1CCCC1]

Compound 255

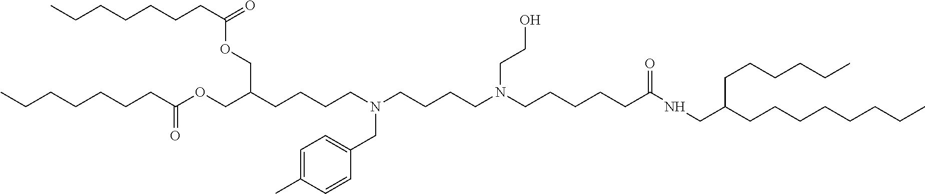 [SMILES: CCCCCCCCC(CCCCCC)CNC(=O)CCCCCN(CCO)CCCCN(CCCCC(COC(=O)CCCCCCC)COC(=O)CCCCCCC)Cc1ccc(C)cc1]

Compound 256

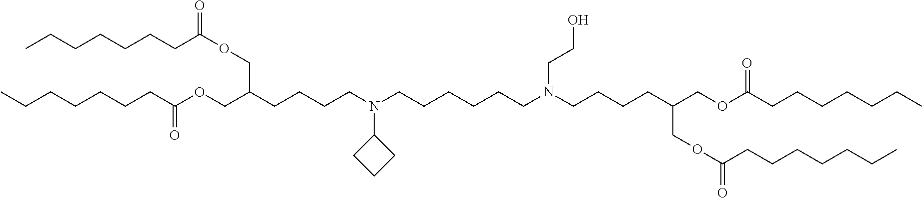 [SMILES: CCCCCCCC(=O)OCC(CCCCN(CCO)CCCCCCN(CCCCC(COC(=O)CCCCCCC)COC(=O)CCCCCCC)C1CCC1)COC(=O)CCCCCCC]

Compound 257

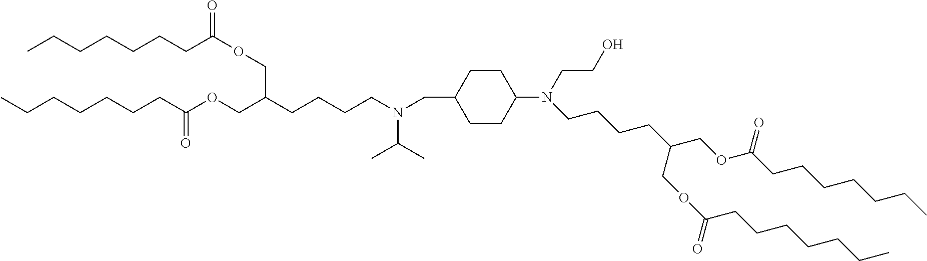 [SMILES: CCCCCCCC(=O)OCC(CCCCN(CC1CCC(N(CCO)CCCCC(COC(=O)CCCCCCC)COC(=O)CCCCCCC)CC1)C(C)C)COC(=O)CCCCCCC]

Compound 258

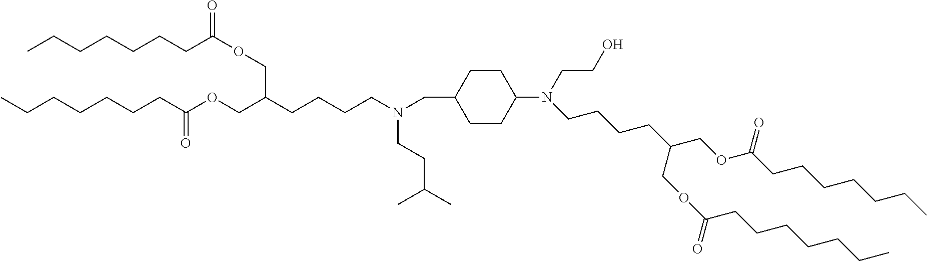 [SMILES: CCCCCCCC(=O)OCC(CCCCN(CCC(C)C)CC1CCC(N(CCO)CCCCC(COC(=O)CCCCCCC)COC(=O)CCCCCCC)CC1)COC(=O)CCCCCCC]

Compound 259

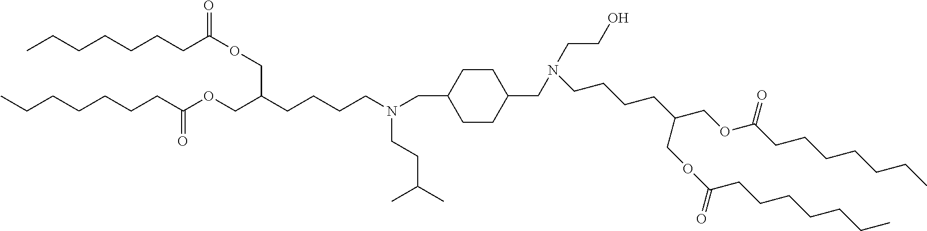 [SMILES: CCCCCCCC(=O)OCC(CCCCN(CCO)CC1CCC(CN(CCCCC(COC(=O)CCCCCCC)COC(=O)CCCCCCC)CCC(C)C)CC1)COC(=O)CCCCCCC]

Compound 260

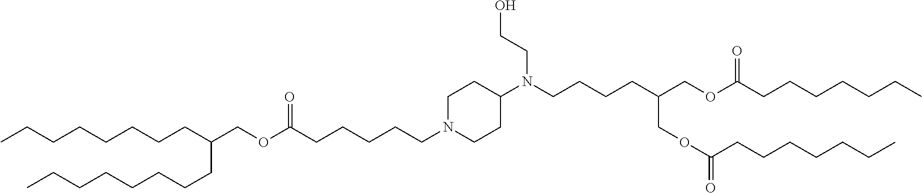 [SMILES: CCCCCCCCC(CCCCCCCC)COC(=O)CCCCCN1CCC(N(CCO)CCCCC(COC(=O)CCCCCCC)COC(=O)CCCCCCC)CC1]

Compound 261

In one embodiment, the compound is a compound in Table 1A, or a pharmaceutically acceptable salt, prodrug or stereoisomer thereof.

TABLE 1A

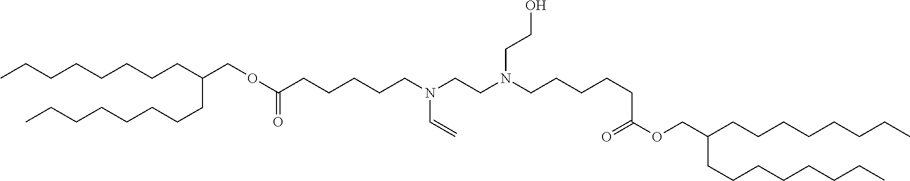 [SMILES: C=CN(CCCCCC(=O)OCC(CCCCCCCC)CCCCCCCC)CCN(CCO)CCCCCC(=O)OCC(CCCCCCCC)CCCCCCCC]

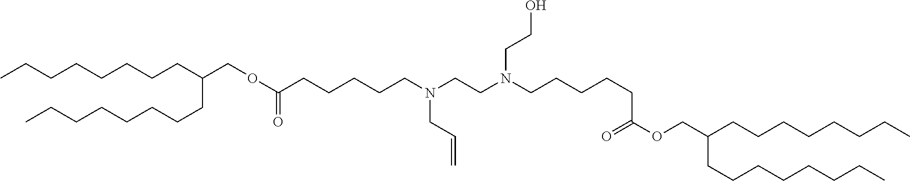 [SMILES: C=CCN(CCCCCC(=O)OCC(CCCCCCCC)CCCCCCCC)CCN(CCO)CCCCCC(=O)OCC(CCCCCCCC)CCCCCCCC]

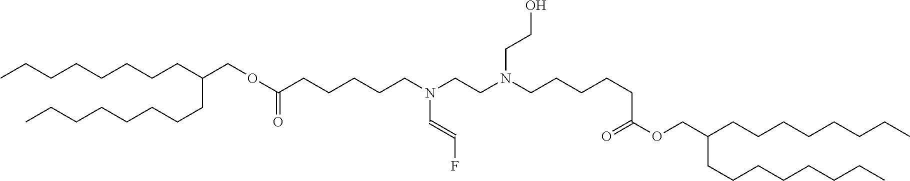 [SMILES: CCCCCCCCC(CCCCCCCC)COC(=O)CCCCCN(/C=C/F)CCN(CCO)CCCCCC(=O)OCC(CCCCCCCC)CCCCCCCC]

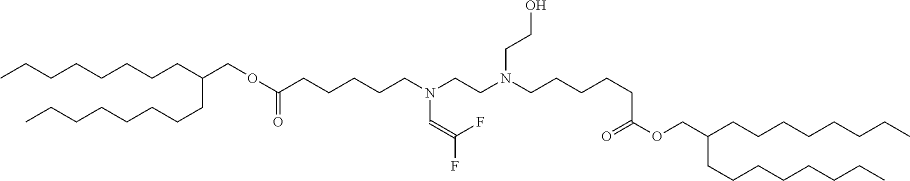 [SMILES: CCCCCCCCC(CCCCCCCC)COC(=O)CCCCCN(C=C(F)F)CCN(CCO)CCCCCC(=O)OCC(CCCCCCCC)CCCCCCCC]

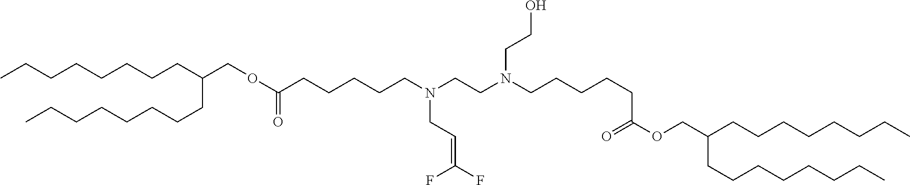 [SMILES: CCCCCCCCC(CCCCCCCC)COC(=O)CCCCCN(CC=C(F)F)CCN(CCO)CCCCCC(=O)OCC(CCCCCCCC)CCCCCCCC]

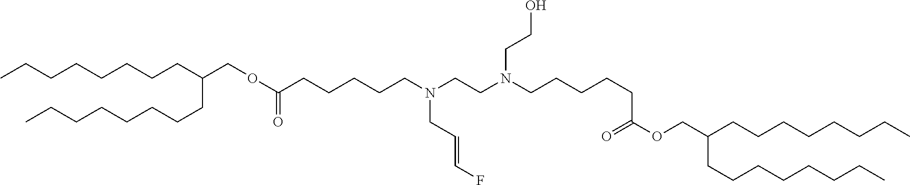 [SMILES: CCCCCCCCC(CCCCCCCC)COC(=O)CCCCCN(C/C=C/F)CCN(CCO)CCCCCC(=O)OCC(CCCCCCCC)CCCCCCCC]

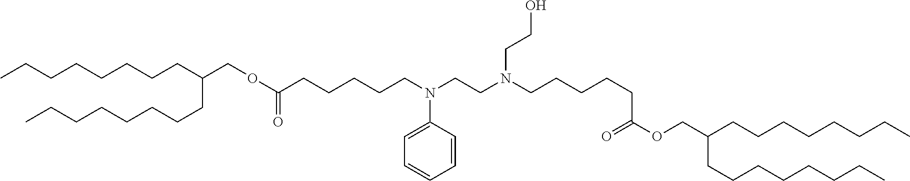 [SMILES: CCCCCCCCC(CCCCCCCC)COC(=O)CCCCCN(CCO)CCN(CCCCCC(=O)OCC(CCCCCCCC)CCCCCCCC)c1ccccc1]

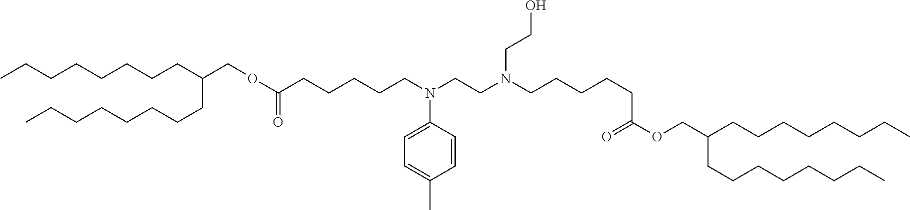 [SMILES: CCCCCCCCC(CCCCCCCC)COC(=O)CCCCCN(CCO)CCN(CCCCCC(=O)OCC(CCCCCCCC)CCCCCCCC)c1ccc(C)cc1]

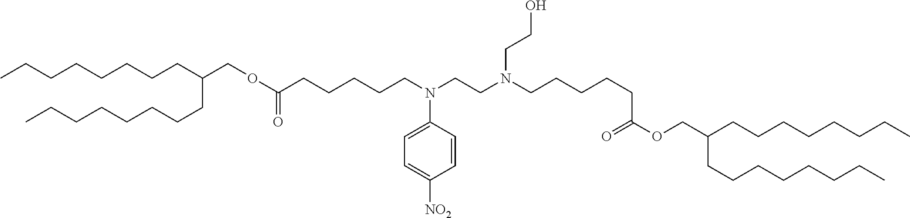 [SMILES: CCCCCCCCC(CCCCCCCC)COC(=O)CCCCCN(CCO)CCN(CCCCCC(=O)OCC(CCCCCCCC)CCCCCCCC)c1ccc([N+](=O)[O-])cc1]

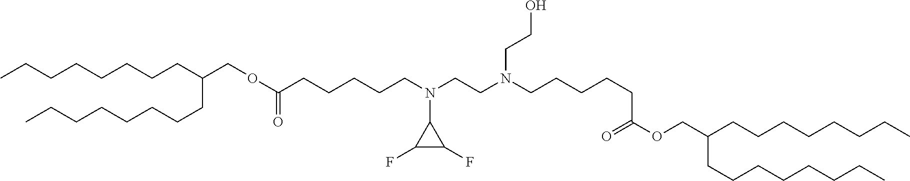 [SMILES: CCCCCCCCC(CCCCCCCC)COC(=O)CCCCCN(CCO)CCN(CCCCCC(=O)OCC(CCCCCCCC)CCCCCCCC)C1C(F)C1F]

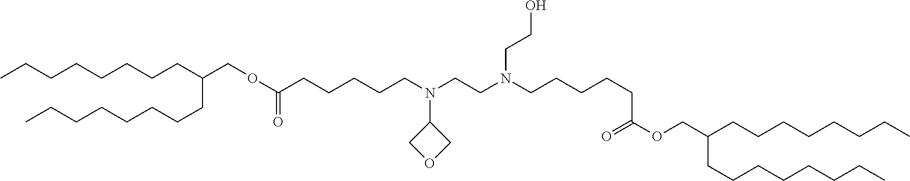 [SMILES: CCCCCCCCC(CCCCCCCC)COC(=O)CCCCCN(CCO)CCN(CCCCCC(=O)OCC(CCCCCCCC)CCCCCCCC)C1COC1]

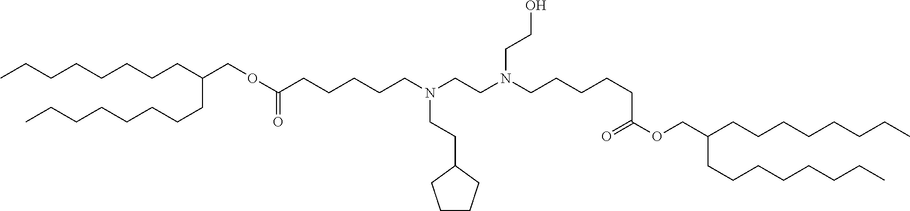 [SMILES: CCCCCCCCC(CCCCCCCC)COC(=O)CCCCCN(CCO)CCN(CCCCCC(=O)OCC(CCCCCCCC)CCCCCCCC)CCC1CCCC1]

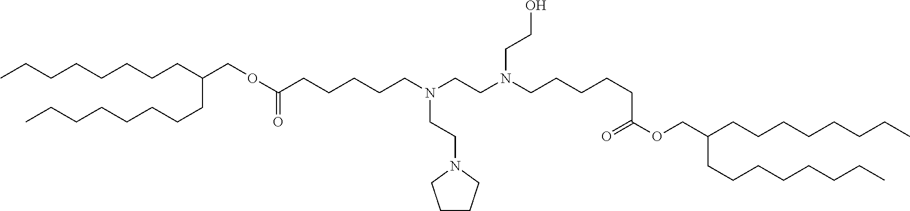 [SMILES: CCCCCCCCC(CCCCCCCC)COC(=O)CCCCCN(CCO)CCN(CCCCCC(=O)OCC(CCCCCCCC)CCCCCCCC)CCN1CCCC1]

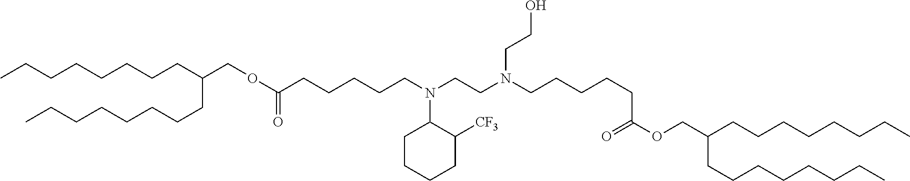 [SMILES: CCCCCCCCC(CCCCCCCC)COC(=O)CCCCCN(CCO)CCN(CCCCCC(=O)OCC(CCCCCCCC)CCCCCCCC)C1CCCCC1C(F)(F)F]

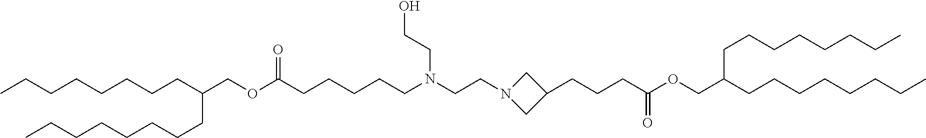 [SMILES: CCCCCCCCC(CCCCCCCC)COC(=O)CCCCCN(CCO)CCN1CC(CCCC(=O)OCC(CCCCCCCC)CCCCCCCC)C1]

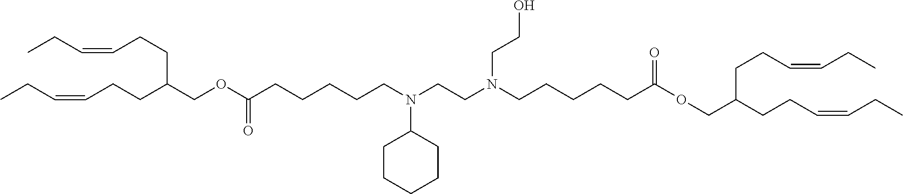 [SMILES: CC/C=C\CCC(CC/C=C\CC)COC(=O)CCCCCN(CCO)CCN(CCCCCC(=O)OCC(CC/C=C\CC)CC/C=C\CC)C1CCCCC1]

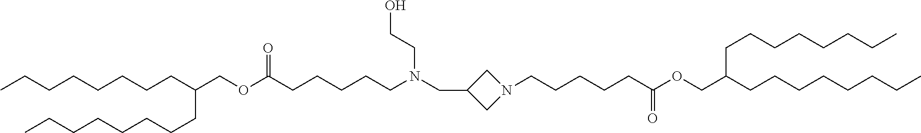 [SMILES: CCCCCCCCC(CCCCCCCC)COC(=O)CCCCCN(CCO)CC1CN(CCCCCC(=O)OCC(CCCCCCCC)CCCCCCCC)C1]

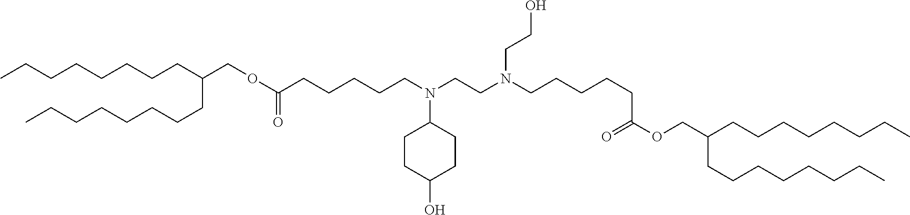 [SMILES: CCCCCCCCC(CCCCCCCC)COC(=O)CCCCCN(CCO)CCN(CCCCCC(=O)OCC(CCCCCCCC)CCCCCCCC)C1CCC(O)CC1]

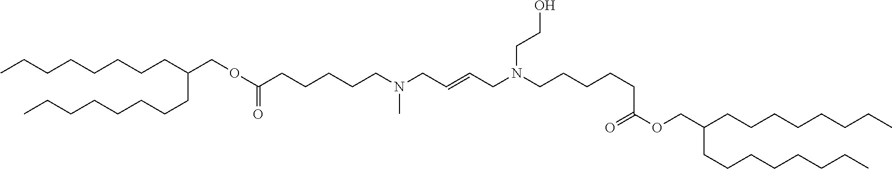 [SMILES: CCCCCCCCC(CCCCCCCC)COC(=O)CCCCCN(C)C/C=C/CN(CCO)CCCCCC(=O)OCC(CCCCCCCC)CCCCCCCC]

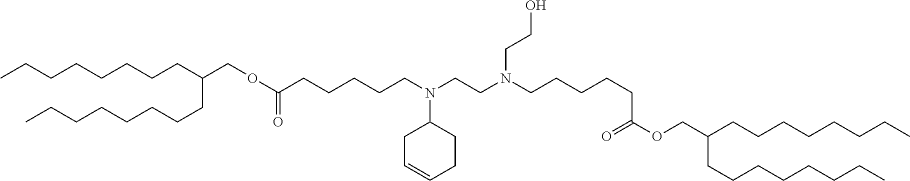 [SMILES: CCCCCCCCC(CCCCCCCC)COC(=O)CCCCCN(CCO)CCN(CCCCCC(=O)OCC(CCCCCCCC)CCCCCCCC)C1CC=CCC1]

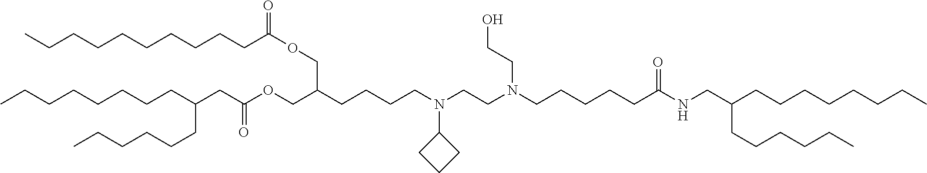 [SMILES: CCCCCCCCCCC(=O)OCC(CCCCN(CCN(CCO)CCCCCC(=O)NCC(CCCCCC)CCCCCCCC)C1CCC1)COC(=O)CC(CCCCCC)CCCCCCCC]

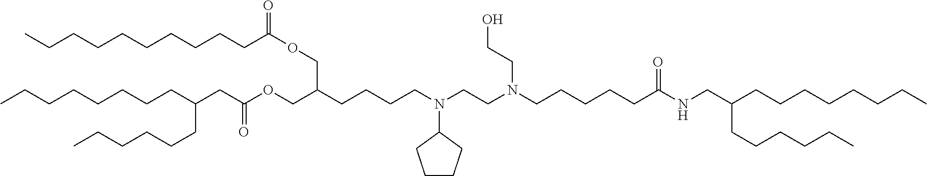 [SMILES: CCCCCCCCCCC(=O)OCC(CCCCN(CCN(CCO)CCCCCC(=O)NCC(CCCCCC)CCCCCCCC)C1CCCC1)COC(=O)CC(CCCCCC)CCCCCCCC]

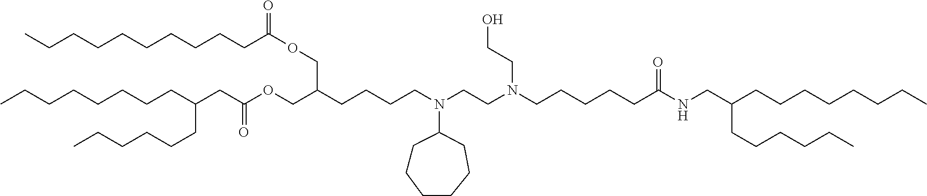 [SMILES: CCCCCCCCCCC(=O)OCC(CCCCN(CCN(CCO)CCCCCC(=O)NCC(CCCCCC)CCCCCCCC)C1CCCCCC1)COC(=O)CC(CCCCCC)CCCCCCCC]

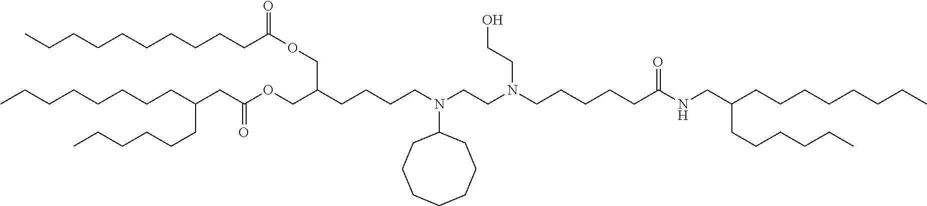 [SMILES: CCCCCCCCCCC(=O)OCC(CCCCN(CCN(CCO)CCCCCC(=O)NCC(CCCCCC)CCCCCCCC)C1CCCCCCC1)COC(=O)CC(CCCCCC)CCCCCCCC]

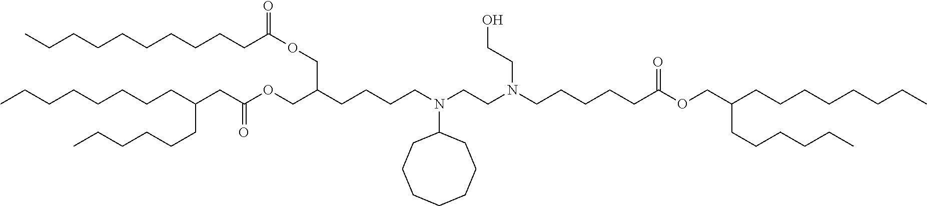 [SMILES: CCCCCCCCCCC(=O)OCC(CCCCN(CCN(CCO)CCCCCC(=O)OCC(CCCCCC)CCCCCCCC)C1CCCCCCC1)COC(=O)CC(CCCCCC)CCCCCCCC]

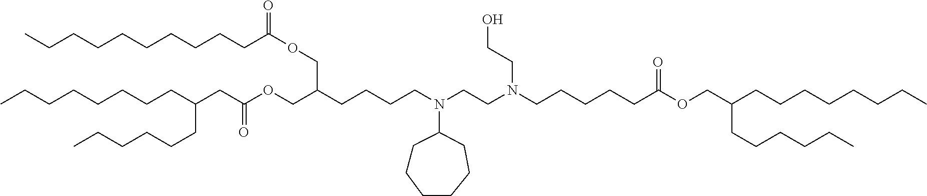 [SMILES: CCCCCCCCCCC(=O)OCC(CCCCN(CCN(CCO)CCCCCC(=O)OCC(CCCCCC)CCCCCCCC)C1CCCCCC1)COC(=O)CC(CCCCCC)CCCCCCCC]

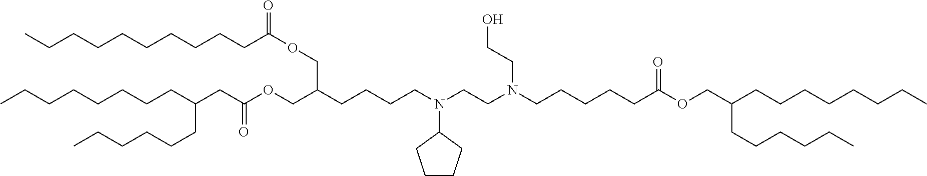 [SMILES: CCCCCCCCCCC(=O)OCC(CCCCN(CCN(CCO)CCCCCC(=O)OCC(CCCCCC)CCCCCCCC)C1CCCC1)COC(=O)CC(CCCCCC)CCCCCCCC]

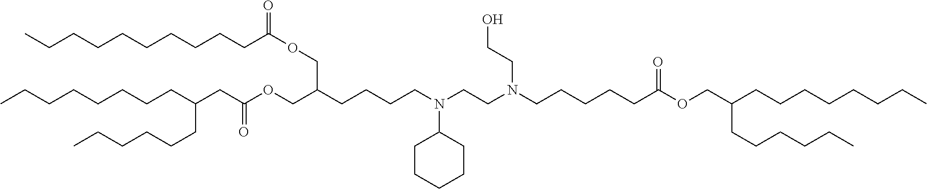 [SMILES: CCCCCCCCCCC(=O)OCC(CCCCN(CCN(CCO)CCCCCC(=O)OCC(CCCCCC)CCCCCCCC)C1CCCCC1)COC(=O)CC(CCCCCC)CCCCCCCC]

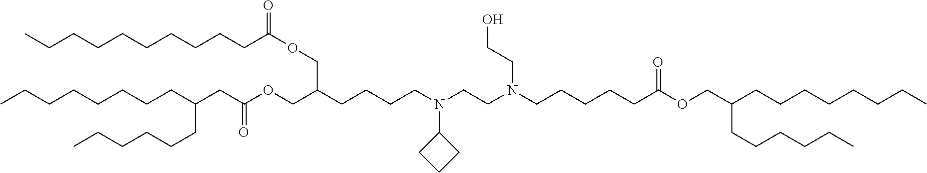 [SMILES: CCCCCCCCCCC(=O)OCC(CCCCN(CCN(CCO)CCCCCC(=O)OCC(CCCCCC)CCCCCCCC)C1CCC1)COC(=O)CC(CCCCCC)CCCCCCCC]

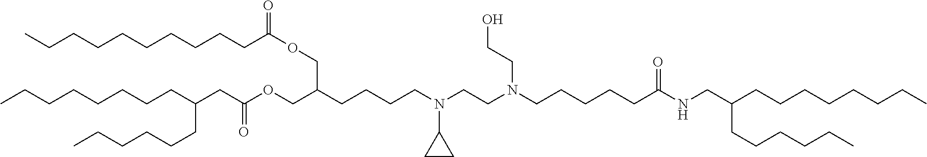 [SMILES: CCCCCCCCCCC(=O)OCC(CCCCN(CCN(CCO)CCCCCC(=O)NCC(CCCCCC)CCCCCCCC)C1CC1)COC(=O)CC(CCCCCC)CCCCCCCC]

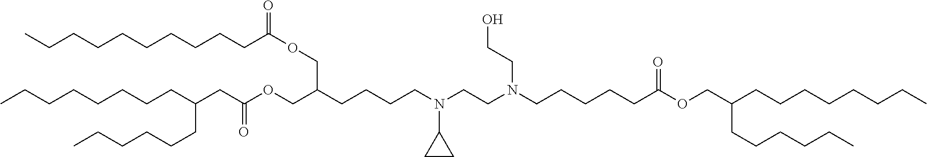 [SMILES: CCCCCCCCCCC(=O)OCC(CCCCN(CCN(CCO)CCCCCC(=O)OCC(CCCCCC)CCCCCCCC)C1CC1)COC(=O)CC(CCCCCC)CCCCCCCC]

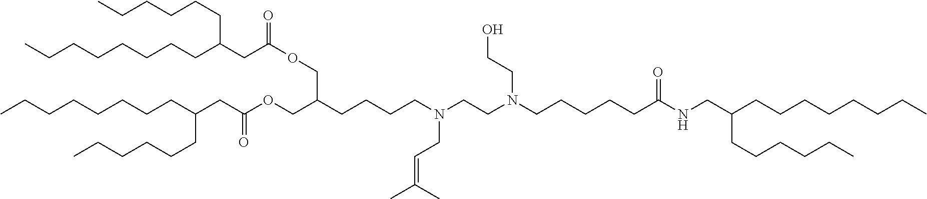 [SMILES: CCCCCCCCC(CCCCCC)CNC(=O)CCCCCN(CCO)CCN(CC=C(C)C)CCCCC(COC(=O)CC(CCCCCC)CCCCCCCC)COC(=O)CC(CCCCCC)CCCCCCCC]

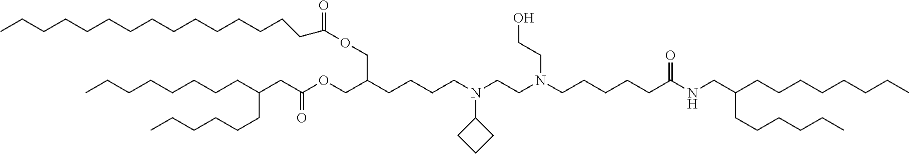 [SMILES: CCCCCCCCCCCCCCCC(=O)OCC(CCCCN(CCN(CCO)CCCCCC(=O)NCC(CCCCCC)CCCCCCCC)C1CCC1)COC(=O)CC(CCCCCC)CCCCCCCC]

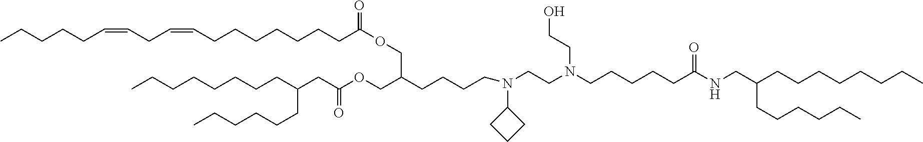 [SMILES: CCCCC/C=C\C/C=C\CCCCCCCC(=O)OCC(CCCCN(CCN(CCO)CCCCCC(=O)NCC(CCCCCC)CCCCCCCC)C1CCC1)COC(=O)CC(CCCCCC)CCCCCCCC]

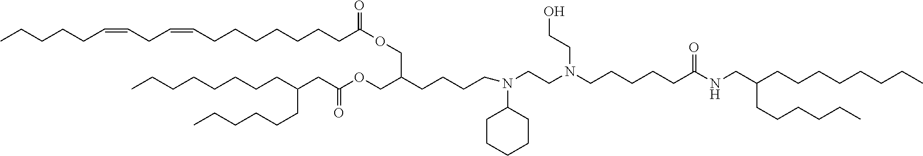 [SMILES: CCCCC/C=C\C/C=C\CCCCCCCC(=O)OCC(CCCCN(CCN(CCO)CCCCCC(=O)NCC(CCCCCC)CCCCCCCC)C1CCCCC1)COC(=O)CC(CCCCCC)CCCCCCCC]

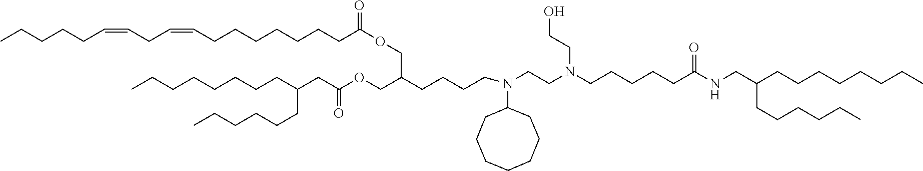 [SMILES: CCCCC/C=C\C/C=C\CCCCCCCC(=O)OCC(CCCCN(CCN(CCO)CCCCCC(=O)NCC(CCCCCC)CCCCCCCC)C1CCCCCCC1)COC(=O)CC(CCCCCC)CCCCCCCC]

In one embodiment, provided herein is a compound of Formula (X):

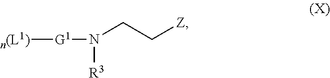

or a pharmaceutically acceptable salt, prodrug or stereoisomer thereof, wherein:
G¹ is a bond, C₂-C₁₂ alkylene, or C₂-C₁₂ alkenylene;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
R¹ is C₆-C₂₄ alkyl or C₆-C₂₄ alkenyl;
R^{a} and R^{b} are each independently H, C₁-C₁₂ alkyl, or C₂-C₁₂ alkenyl;
R^{c} is C₁-C₂₄ alkyl or C₂-C₂₄ alkenyl;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety;
x is 0, 1, or 2;
n is 1 or 2; and
Z is —OH or halogen;
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

In one embodiment, Z is —OH. In one embodiment, Z is halogen. In one embodiment, Z is —Cl.

In one embodiment, a compound of Formula (X) is an intermediate used in a process of preparing a compound of Formula (I), e.g., as exemplified in the examples provided herein.

It is understood that any embodiment of the compounds provided herein, as set forth above, and any specific substituent and/or variable in the compound provided herein, as set forth above, may be independently combined with other embodiments and/or substituents and/or variables of the compounds to form embodiments not specifically set forth above. In addition, in the event that a list of substituents and/or variables is listed for any particular group or variable, it is understood that each individual substituent and/or variable may be deleted from the particular embodiment and/or claim and that the remaining list of substituents and/or variables will be considered to be within the scope of embodiments provided herein.

It is understood that in the present description, combinations of substituents and/or variables of the depicted formulae are permissible only if such contributions result in stable compounds.

### 5.4 Nanoparticle Compositions

In one aspect, described herein are nanoparticle compositions comprising a lipid compound described herein. In particular embodiments, the nanoparticle composition comprises a compound according to Formulae (I) (and sub-formulas thereof) as described herein.

In some embodiments, the largest dimension of a nanoparticle composition provided herein is 1 μm or shorter (e.g., ≤1 μm, ≤900 nm, ≤800 nm, ≤700 nm, ≤600 nm, ≤500 nm, ≤400 nm, ≤300 nm, ≤200 nm, ≤175 nm, ≤150 nm, ≤125 nm, ≤100 nm, ≤75 nm, ≤50 nm, or shorter), such as when measured by dynamic light scattering (DLS), transmission electron microscopy, scanning electron microscopy, or another method. In one embodiment, the lipid nanoparticle provided herein has at least one dimension that is in the range of from about 40 to about 200 nm. In one embodiment, the at least one dimension is in the range of from about 40 to about 100 nm.

Nanoparticle compositions that can be used in connection with the present disclosure include, for example, lipid nanoparticles (LNPs), nano liproprotein particles, liposomes, lipid vesicles, and lipoplexes. In some embodiments, nanoparticle compositions are vesicles including one or more lipid bilayers. In some embodiments, a nanoparticle composition includes two or more concentric bilayers separated by aqueous compartments. Lipid bilayers may be functionalized and/or crosslinked to one another. Lipid bilayers may include one or more ligands, proteins, or channels.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, and Worcestershire, UK) may also be used to measure multiple characteristics of a nanoparticle composition, such as particle size, polydispersity index, and zeta potential.

Dh (size): The mean size of a nanoparticle composition may be between 10s of nm and 100s of nm. For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In some embodiments, the mean size may be about 80 nm. In other embodiments, the mean size may be about 100 nm.

PDI: A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

Encapsulation Efficiency: The efficiency of encapsulation of a therapeutic and/or prophylactic agent describes the amount of therapeutic and/or prophylactic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic agent in a solution containing the nanoparticle composition before and after breaking up the nanoparticle composition with one or more organic solvents or detergents. Fluorescence may be used to measure the amount of free therapeutic and/or prophylactic agent (e.g., RNA) in a solution. For the nanoparticle compositions described herein, the encapsulation efficiency of a therapeutic and/or prophylactic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Apparent pKa: The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In another embodiment, the self-replicating RNA may be formulated in a liposome. As a non-limiting example, the self-replicating RNA may be formulated in liposomes as described in International Publication No. WO20120067378, herein incorporated by reference in its entirety. In one aspect, the liposomes may comprise lipids which have a pKa value which may be advantageous for delivery of mRNA. In another aspect, the liposomes may have an essentially neutral Surface charge at physiological pH and may therefore be effective for immunization (see e.g., the liposomes described in International Publication No. WO20120067378, herein incorporated by reference in its entirety).

In some embodiments, nanoparticle compositions as described comprise a lipid component including at least one lipid, such as a compound according to one of Formulae (I) (and sub-formulas thereof) as described herein. For example, in some embodiments, a nanoparticle composition may include a lipid component including one of compounds provided herein. Nanoparticle compositions may also include one or more other lipid or non-lipid components as described below.

In one embodiment, a nanoparticle composition comprising a compound provided herein and an mRNA shows improved expression level of the mRNA (e.g., as compared to standard cationic lipid compounds known in the art, e.g., MC3). In one embodiment, after a nanoparticle composition comprising a compound provided herein is administered to a subject, the compound shows a rapid tissue clearance (e.g., liver clearance).

### 5.4.1 Cationic/Ionizable Lipids

As described herein, in some embodiments, a nanoparticle composition provided herein comprises one or more charged or ionizable lipids in addition to a lipid according Formulae (I) (and sub-formulas thereof). Without being bound by the theory, it is contemplated that certain charged or zwitterionic lipid components of a nanoparticle composition resembles the lipid component in the cell membrane, thereby can improve cellular uptake of the nanoparticle. Exemplary charged or ionizable lipids that can form part of the present nanoparticle composition include but are not limited to 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLinDMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3 [(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3β)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), (12Z,15Z)—N,N-dimethyl-2-nonylhenicosa-12,15-den-1-amine, N,N-dimethyl-1-{(1S,2R)-2-octylcyclopropyl}heptadecan-8-amine. Additional exemplary charged or ionizable lipids that can form part of the present nanoparticle composition include the lipids (e.g., lipid 5) described in Sabnis et al. “A Novel Amino Lipid Series for mRNA Delivery: Improved Endosomal Escape and Sustained Pharmacology and Safety in Non-human Primates”, Molecular Therapy Vol. 26 No 6, 2018, the entirety of which is incorporated herein by reference.

In some embodiments, suitable cationic lipids include N-[1-(2,3-dioleyloxy)propyl]-N,N,N-trimethylammonium chloride (DOTMA); N-[1-(2,3-dioleoyloxy)propyl]-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1); N1-[2-((1S)-1-[(3-aminopropyl)amino]-4-[di(3-amino-propyl)amino]butylcarboxamido)ethyl]-3,4-di[oleyloxy]-benzamide (MVL5); dioctadecylamido-glycylspermine (DOGS); 3b-[N—(N′,N′-dimethylaminoethyl)carbamoyl]cholesterol (DC-Chol); dioctadecyldimethylammonium bromide (DDAB); SAINT-2, N-methyl-4-(dioleyl)methylpyridinium; 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); di-alkylated amino acid (DILA²) (e.g., C18:1-norArg-C16); dioleyldimethylammonium chloride (DODAC); 1-palmitoyl-2-oleoyl-sn-glycero-3-ethylphosphocholine (POEPC); 1,2-dimyristoleoyl-sn-glycero-3-ethylphosphocholine (MOEPC); (R)-5-(dimethylamino)pentane-1,2-diyl dioleate hydrochloride (DODAPen-Cl); (R)-5-guanidinopentane-1,2-diyl dioleate hydrochloride (DOPen-G); and (R)—N,N,N-trimethyl-4,5-bis(oleoyloxy)pentan-1-aminium chloride (DOTAPen). Also suitable are cationic lipids with headgroups that are charged at physiological pH, such as primary amines (e.g., DODAG N′,N′-dioctadecyl-N-4,8-diaza-10-aminodecanoylglycine amide) and guanidinium head groups (e.g., bis-guanidinium-spermidine-cholesterol (BGSC), bis-guanidiniumtren-cholesterol (BGTC), PONA, and (R)-5-guanidinopentane-1,2-diyl dioleate hydrochloride (DOPen-G)). Yet another suitable cationic lipid is (R)-5-(dimethylamino)pentane-1,2-diyl dioleate hydrochloride (DODAPen-Cl). In certain embodiments, the cationic lipid is a particular enantiomer or the racemic form, and includes the various salt forms of a cationic lipid as above (e.g., chloride or sulfate). For example, in some embodiments, the cationic lipid is N-[1-(2,3-dioleoyloxy)propyl]-N,N,N-trimethylammonium chloride (DOTAP-Cl) or N-[1-(2,3-dioleoyloxy)propyl]-N,N,N-trimethylammonium sulfate (DOTAP-Sulfate). In some embodiments, the cationic lipid is an ionizable cationic lipid such as, e.g., dioctadecyldimethylammonium bromide (DDAB); 1,2-dilinoleyloxy-3-dimethylaminopropane (DLinDMA); 2,2-dilinoleyl-4-(2dimethylaminoethyl)-[1,3]-dioxolane (DLin-KC2-DMA); heptatriaconta-6,9,28,31-tetraen-19-yl 4-(dimethylamino)butanoate (DLin-MC3-DMA); 1,2-dioleoyloxy-3-dimethylaminopropane (DODAP); 1,2-dioleyloxy-3-dimethylaminopropane (DODMA); and morpholinocholesterol (Mo-CHOL). In certain embodiments, a lipid nanoparticle includes a combination or two or more cationic lipids (e.g., two or more cationic lipids as above).

Additionally, in some embodiments, the charged or ionizable lipid that can form part of the present nanoparticle composition is a lipid including a cyclic amine group. Additional cationic lipids that are suitable for the formulations and methods disclosed herein include those described in WO2015199952, WO2016176330, and WO2015011633, the entire contents of each of which are hereby incorporated by reference in their entireties.

### 5.4.2 Polymer Conjugated Lipids

In some embodiments, the lipid component of a nanoparticle composition can include one or more polymer conjugated lipids, such as PEGylated lipids (PEG lipids). Without being bound by the theory, it is contemplated that a polymer conjugated lipid component in a nanoparticle composition can improve of colloidal stability and/or reduce protein absorption of the nanoparticles. Exemplary polymer conjugated lipids that can be used in connection with the present disclosure include but are not limited to PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DSPE, Ceramide-PEG2000, or Chol-PEG2000.

In one embodiment, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In one embodiment, the polymer conjugated lipid is present in a concentration ranging from 1.0 to 2.5 molar percent. In one embodiment, the polymer conjugated lipid is present in a concentration of about 1.7 molar percent. In one embodiment, the polymer conjugated lipid is present in a concentration of about 1.5 molar percent.

In one embodiment, the molar ratio of cationic lipid to the polymer conjugated lipid ranges from about 35:1 to about 25:1. In one embodiment, the molar ratio of cationic lipid to polymer conjugated lipid ranges from about 100:1 to about 20:1.

In one embodiment, the pegylated lipid has the following Formula:

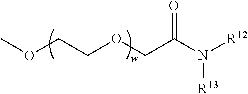

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and
w has a mean value ranging from 30 to 60.

In one embodiment, R¹² and R¹³ are each independently straight, saturated alkyl chains containing from 12 to 16 carbon atoms. In other embodiments, the average w ranges from 42 to 55, for example, the average w is 42, 43, 44, 45, 46, 47, 48, 49, 50, 51, 52, 53, 54 or 55. In some specific embodiments, the average w is about 49.

In one embodiment, the pegylated lipid has the following Formula:

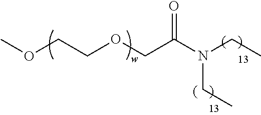

wherein the average w is about 49.

### 5.4.3 Structural Lipids

In some embodiments, the lipid component of a nanoparticle composition can include one or more structural lipids. Without being bound by the theory, it is contemplated that structural lipids can stabilize the amphiphilic structure of a nanoparticle, such as but not limited to the lipid bilayer structure of a nanoparticle. Exemplary structural lipids that can be used in connection with the present disclosure include but are not limited to cholesterol, fecosterol, sitosterol, ergosterol, campesterol, stigmasterol, brassicasterol, tomatidine, tomatine, ursolic acid, alpha-tocopherol, and mixtures thereof. In certain embodiments, the structural lipid is cholesterol. In some embodiments, the structural lipid includes cholesterol and a corticosteroid (such as prednisolone, dexamethasone, prednisone, and hydrocortisone), or a combination thereof.

In one embodiment, the lipid nanoparticles provided herein comprise a steroid or steroid analogue. In one embodiment, the steroid or steroid analogue is cholesterol. In one embodiment, the steroid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent.

In one embodiment, the molar ratio of cationic lipid to the steroid ranges from 1.0:0.9 to 1.0:1.2, or from 1.0:1.0 to 1.0:1.2. In one embodiment, the molar ratio of cationic lipid to cholesterol ranges from about 5:1 to 1:1. In one embodiment, the steroid is present in a concentration ranging from 32 to 40 mol percent of the steroid.

### 5.4.4 Phospholipids

In some embodiments, the lipid component of a nanoparticle composition can include one or more phospholipids, such as one or more (poly)unsaturated lipids. Without being bound by the theory, it is contemplated that phospholipids may assemble into one or more lipid bilayers structures. Exemplary phospholipids that can form part of the present nanoparticle composition include but are not limited to 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In certain embodiments, a nanoparticle composition includes DSPC. In certain embodiments, a nanoparticle composition includes DOPE. In some embodiments, a nanoparticle composition includes both DSPC and DOPE.

Additional exemplary neutral lipids include, for example, dipalmitoylphosphatidylglycerol (DPPG), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC). In one embodiment, the neutral lipid is selected from DSPC, DPPC, DMPC, DOPC, POPC, DOPE and SM.

In one embodiment, the neutral lipid is phosphatidylcholine (PC), phosphatidylethanolamine (PE) phosphatidylserine (PS), phosphatidic acid (PA), or phosphatidylglycerol (PG).

Additionally phospholipids that can form part of the present nanoparticle composition also include those described in WO2017/112865, the entire content of which is hereby incorporated by reference in its entirety.

### 5.4.5 Therapeutic Payload

According to the present disclosure, nanoparticle compositions as described herein can further comprise one or more therapeutic and/or prophylactic agents. These therapeutic and/or prophylactic agents are sometimes referred to as a “therapeutic payload” or “payload” in the present disclosure. In some embodiments, the therapeutic payload can be administered in vivo or in vitro using the nanoparticles as a delivery vehicle.

In some embodiments, the nanoparticle composition comprises, as the therapeutic payload, a small molecule compound (e.g., a small molecule drug) such as antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cytosine arabinoside, anthracyclines, alkylating agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetalol), antihypertensive agents (e.g., clonidine and hydralazine), anti-depressants (e.g., imipramine, amitriptyline, and doxepin), anti-convulsants (e.g., phenytoin), antihistamines (e.g., diphenhydramine, chlorpheniramine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some embodiments, the therapeutic payload comprises a cytotoxin, a radioactive ion, a chemotherapeutic, a vaccine, a compound that elicits an immune response, and/or another therapeutic and/or prophylactic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol, rachelmycin (CC-1065), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium.

In other embodiments, the therapeutic payload of the present nanoparticle composition can include, but is not limited to, therapeutic and/or prophylactic agents such as antimetabolites (e.g., methotrexate, 6-mercaptopurine, 6-thioguanine, cytarabine, 5-fluorouracil, dacarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the nanoparticle composition comprises, as the therapeutic payload, a biological molecule such as peptides and polypeptides. The biological molecules forming part of the present nanoparticle composition can be either of a natural source or synthetic. For example, in some embodiments, the therapeutic payload of the present nanoparticle composition can include, but is not limited to gentamycin, amikacin, insulin, erythropoietin (EPO), granulocyte-colony stimulating factor (G-CSF), granulocyte-macrophage colony stimulating factor (GM-CSF), Factor VIR, luteinizing hormone-releasing hormone (LHRH) analogs, interferons, heparin, Hepatitis B surface antigen, typhoid vaccine, cholera vaccine, and peptides and polypeptides.

### 5.4.5.1 Nucleic Acids

In some embodiments, the present nanoparticle composition comprises one or more nucleic acid molecules (e.g., DNA or RNA molecules) as the therapeutic payload. Exemplary forms of nucleic acid molecules that can be included in the present nanoparticle composition as therapeutic payload include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, the therapeutic payload comprises an RNA. RNA molecules that can be included in the present nanoparticle composition as the therapeutic payload include, but are not limited to, shortmers, agomirs, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and other forms of RNA molecules known in the art. In particular embodiments, the RNA is an mRNA.

In other embodiments, the nanoparticle composition comprises a siRNA molecule as the therapeutic payload. Particularly, in some embodiments, the siRNA molecule is capable of selectively interfering with and downregulate the expression of a gene of interest. For example, in some embodiments, the siRNA payload selectively silence a gene associated with a particular disease, disorder, or condition upon administration to a subject in need thereof of a nanoparticle composition including the siRNA. In some embodiments, the siRNA molecule comprises a sequence that is complementary to an mRNA sequence encoding a protein product of interest. In some embodiments, the siRNA molecule is an immunomodulatory siRNA.

In some embodiments, the nanoparticle composition comprises a shRNA molecule or a vector encoding the shRNA molecule as the therapeutic payload. Particularly, in some embodiments, the therapeutic payload, upon administering to a target cell, produces shRNA inside the target cell. Constructs and mechanisms relating to shRNA are well known in the relevant arts.

In some embodiments, the nanoparticle composition comprises an mRNA molecule as the therapeutic payload. Particularly, in some embodiments, the mRNA molecule encodes a polypeptide of interest, including any naturally or non-naturally occurring or otherwise modified polypeptide. A polypeptide encoded by an mRNA may be of any size and may have any secondary structure or activity. In some embodiments, the polypeptide encoded by an mRNA payload can have a therapeutic effect when expressed in a cell.

In some embodiment, a nucleic acid molecule of the present disclosure comprises an mRNA molecule. In specific embodiments, the nucleic acid molecule comprises at least one coding region encoding a peptide or polypeptide of interest (e.g., an open reading frame (ORF)). In some embodiments, the nucleic acid molecule further comprises at least one untranslated region (UTR). In particular embodiments, the untranslated region (UTR) is located upstream (to the 5′-end) of the coding region, and is referred to herein as the 5′-UTR. In particular embodiments, the untranslated region (UTR) is located downstream (to the 3′-end) of the coding region, and is referred to herein as the 3′-UTR. In particular embodiments, the nucleic acid molecule comprises both a 5′-UTR and a 3′-UTR. In some embodiments, the 5′-UTR comprises a 5′-Cap structure. In some embodiments, the nucleic acid molecule comprises a Kozak sequence (e.g., in the 5′-UTR). In some embodiments, the nucleic acid molecule comprises a poly-A region (e.g., in the 3′-UTR). In some embodiments, the nucleic acid molecule comprises a polyadenylation signal (e.g., in the 3′-UTR). In some embodiments, the nucleic acid molecule comprises stabilizing region (e.g., in the 3′-UTR). In some embodiments, the nucleic acid molecule comprises a secondary structure. In some embodiments, the secondary structure is a stem-loop. In some embodiments, the nucleic acid molecule comprises a stem-loop sequence (e.g., in the 5′-UTR and/or the 3′-UTR). In some embodiments, the nucleic acid molecule comprises one or more intronic regions capable of being excised during splicing. In a specific embodiment, the nucleic acid molecule comprises one or more region selected from a 5′-UTR, and a coding region. In a specific embodiment, the nucleic acid molecule comprises one or more region selected from a coding region and a 3′-UTR. In a specific embodiment, the nucleic acid molecule comprises one or more region selected from a 5′-UTR, a coding region, and a 3′-UTR.

### Coding Region

In some embodiments, the nucleic acid molecule of the present disclosure comprises at least one coding region. In some embodiments, the coding region is an open reading frame (ORF) that encodes for a single peptide or protein. In some embodiments, the coding region comprises at least two ORFs, each encoding a peptide or protein. In those embodiments where the coding region comprises more than one ORFs, the encoded peptides and/or proteins can be the same as or different from each other. In some embodiments, the multiple ORFs in a coding region are separated by non-coding sequences. In specific embodiments, a non-coding sequence separating two ORFs comprises an internal ribosome entry sites (IRES).

Without being bound by the theory, it is contemplated that an internal ribosome entry sites (IRES) can act as the sole ribosome binding site, or serve as one of multiple ribosome binding sites of an mRNA. An mRNA molecule containing more than one functional ribosome binding site can encode several peptides or polypeptides that are translated independently by the ribosomes (e.g., multicistronic mRNA). Accordingly, in some embodiments, the nucleic acid molecule of the present disclosure (e.g., mRNA) comprises one or more internal ribosome entry sites (IRES). Examples of IRES sequences that can be used in connection with the present disclosure include, without limitation, those from picomaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In various embodiments, the nucleic acid molecule of the present disclose encodes for at least 1, 2, 3, 4, 5, 6, 7, 8, 9, 10 or more than 10 peptides or proteins. Peptides and proteins encoded by a nucleic acid molecule can be the same or different. In some embodiments, the nucleic acid molecule of the present disclosure encodes a dipeptide (e.g., camosine and anserine). In some embodiments, the nucleic acid molecule encodes a tripeptide. In some embodiments, the nucleic acid molecule encodes a tetrapeptide. In some embodiments, the nucleic acid molecule encodes a pentapeptide. In some embodiments, the nucleic acid molecule encodes a hexapeptide. In some embodiments, the nucleic acid molecule encodes a heptapeptide. In some embodiments, the nucleic acid molecule encodes an octapeptide. In some embodiments, the nucleic acid molecule encodes a nonapeptide. In some embodiments, the nucleic acid molecule encodes a decapeptide. In some embodiments, the nucleic acid molecule encodes a peptide or polypeptide that has at least about 15 amino acids. In some embodiments, the nucleic acid molecule encodes a peptide or polypeptide that has at least about 50 amino acids. In some embodiments, the nucleic acid molecule encodes a peptide or polypeptide that has at least about 100 amino acids. In some embodiments, the nucleic acid molecule encodes a peptide or polypeptide that has at least about 150 amino acids. In some embodiments, the nucleic acid molecule encodes a peptide or polypeptide that has at least about 300 amino acids. In some embodiments, the nucleic acid molecule encodes a peptide or polypeptide that has at least about 500 amino acids. In some embodiments, the nucleic acid molecule encodes a peptide or polypeptide that has at least about 1000 amino acids.

In some embodiments, the nucleic acid molecule of the present disclosure is at least about 30 nucleotides (nt) in length. In some embodiments, the nucleic acid molecule is at least about 35 nt in length. In some embodiments, the nucleic acid molecule is at least about 40 nt in length. In some embodiments, the nucleic acid molecule is at least about 45 nt in length. In some embodiments the nucleic acid molecule is at least about 50 nt in length. In some embodiments, the nucleic acid molecule is at least about 55 nt in length. In some embodiments, the nucleic acid molecule is at least about 60 nt in length. In some embodiments, the nucleic acid molecule is at least about 65 nt in length. In some embodiments, the nucleic acid molecule is at least about 70 nt in length. In some embodiments, the nucleic acid molecule is at least about 75 nt in length. In some embodiments, the nucleic acid molecule is at least about 80 nt in length. In some embodiments the nucleic acid molecule is at least about 85 nt in length. In some embodiments, the nucleic acid molecule is at least about 90 nt in length. In some embodiments, the nucleic acid molecule is at least about 95 nt in length. In some embodiments, the nucleic acid molecule is at least about 100 nt in length. In some embodiments, the nucleic acid molecule is at least about 120 nt in length. In some embodiments, the nucleic acid molecule is at least about 140 nt in length. In some embodiments, the nucleic acid molecule is at least about 160 nt in length. In some embodiments, the nucleic acid molecule is at least about 180 nt in length. In some embodiments, the nucleic acid molecule is at least about 200 nt in length. In some embodiments, the nucleic acid molecule is at least about 250 nt in length. In some embodiments, the nucleic acid molecule is at least about 300 nt in length. In some embodiments, the nucleic acid molecule is at least about 400 nt in length. In some embodiments, the nucleic acid molecule is at least about 500 nt in length. In some embodiments, the nucleic acid molecule is at least about 600 nt in length. In some embodiments, the nucleic acid molecule is at least about 700 nt in length. In some embodiments, the nucleic acid molecule is at least about 800 nt in length. In some embodiments, the nucleic acid molecule is at least about 900 nt in length. In some embodiments, the nucleic acid molecule is at least about 1000 nt in length. In some embodiments, the nucleic acid molecule is at least about 1100 nt in length. In some embodiments, the nucleic acid molecule is at least about 1200 nt in length. In some embodiments, the nucleic acid molecule is at least about 1300 nt in length. In some embodiments, the nucleic acid molecule is at least about 1400 nt in length. In some embodiments, the nucleic acid molecule is at least about 1500 nt in length. In some embodiments, the nucleic acid molecule is at least about 1600 nt in length. In some embodiments, the nucleic acid molecule is at least about 1700 nt in length. In some embodiments, the nucleic acid molecule is at least about 1800 nt in length. In some embodiments, the nucleic acid molecule is at least about 1900 nt in length. In some embodiments, the nucleic acid molecule is at least about 2000 nt in length. In some embodiments, the nucleic acid molecule is at least about 2500 nt in length. In some embodiments, the nucleic acid molecule is at least about 3000 nt in length. In some embodiments, the nucleic acid molecule is at least about 3500 nt in length. In some embodiments, the nucleic acid molecule is at least about 4000 nt in length. In some embodiments, the nucleic acid molecule is at least about 4500 nt in length. In some embodiments, the nucleic acid molecule is at least about 5000 nt in length.

In specific embodiments, the therapeutic payload comprises a vaccine composition (e.g., a genetic vaccine) as described herein. In some embodiments, the therapeutic payload comprises a compound capable of eliciting immunity against one or more target conditions or disease. In some embodiments, the target condition is related to or caused by infection by a pathogen, such as a coronavirus (e.g. 2019-nCoV), influenza, measles, human papillomavirus (HPV), rabies, meningitis, whooping cough, tetanus, plague, hepatitis, and tuberculosis. In some embodiments, the therapeutic payload comprises a nucleic acid sequence (e.g., mRNA) encoding a pathogenic protein characteristic for the pathogen, or an antigenic fragment or epitope thereof. The vaccine, upon administration to a vaccinated subject, allows for expression of the encoded pathogenic protein (or the antigenic fragment or epitope thereof), thereby eliciting immunity in the subject against the pathogen.

In some embodiments, the target condition is related to or caused by neoplastic growth of cells, such as a cancer. In some embodiments, the therapeutic payload comprises a nucleic acid sequence (e.g., mRNA) encoding a tumor associated antigen (TAA) characteristic for the cancer, or an antigenic fragment or epitope thereof. The vaccine, upon administration to a vaccinated subject, allows for expression of the encoded TAA (or the antigenic fragment or epitope thereof), thereby eliciting immunity in the subject against the neoplastic cells expressing the TAA.

### 5′-Cap Structure

Without being bound by the theory, it is contemplated that, a 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The 5′-cap structure further assists the removal of 5′-proximal introns removal during mRNA splicing. Accordingly, in some embodiments, the nucleic acid molecules of the present disclosure comprise a 5′-cap structure.

Nucleic acid molecules may be 5′-end capped by the endogenous transcription machinery of a cell to generate a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a nucleic acid molecule, such as an mRNA molecule, for degradation.

In some embodiments, the nucleic acid molecules of the present disclosure comprise one or more alterations to the natural 5′-cap structure generated by the endogenous process. Without being bound by the theory, a modification on the 5′-cap may increase the stability of polynucleotide, increase the half-life of the polynucleotide, and could increase the polynucleotide translational efficiency.

Exemplary alterations to the natural 5′-Cap structure include generation of a non-hydrolyzable cap structure preventing decapping and thus increasing polynucleotide half-life. In some embodiments, because cap structure hydrolysis requires cleavage of 5′-ppp-5′ phosphorodiester linkages, in some embodiments, modified nucleotides may be used during the capping reaction. For example, in some embodiments, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used, such as α-methyl-phosphonate and seleno-phosphate nucleotides.

Additional exemplary alterations to the natural 5′-Cap structure also include modification at the 2′- and/or 3′-position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

Additional exemplary alterations to the natural 5′-cap structure include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule. Additional exemplary 5′-Cap structures that can be used in connection with the present disclosure further include those described in International Patent Publication Nos. WO2008127688, WO 2008016473, and WO 2011015347, the entire contents of each of which are incorporated herein by reference.

In various embodiments, 5′-terminal caps can include cap analogs. Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,3′-O-dimethyl-guanosine-5′-triphosphate-5′-guanosine, m⁷G-3′mppp-G, which may equivalently be designated 3′ 0-Me-m7G(5′)ppp(5′)G). The 3′-0 atom of the other, unaltered, guanosine becomes linked to the 5′-terminal nucleotide of the capped polynucleotide (e.g., an mRNA). The N7- and 3′-O-methlyated guanosine provides the terminal moiety of the capped polynucleotide (e.g., mRNA). Another exemplary cap structure is mCAP, which is similar to ARCA but has a 2′-O-methyl group on guanosine (i.e., N7,2′-O-dimethyl-guanosine-5′-triphosphate-5′-guanosine, m₇Gm-ppp-G).

In some embodiments, a cap analog can be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the entire content of which is herein incorporated by reference in its entirety.

In some embodiments, a cap analog can be a N7-(4-chlorophenoxyethyl) substituted dinucleotide cap analog known in the art and/or described herein. Non-limiting examples of N7-(4-chlorophenoxyethyl) substituted dinucleotide cap analogs include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574; the entire content of which is herein incorporated by reference). In other embodiments, a cap analog useful in connection with the nucleic acid molecules of the present disclosure is a 4-chloro/bromophenoxyethyl analog.

In various embodiments, a cap analog can include a guanosine analog. Useful guanosine analogs include but are not limited to inosine, N1-methyl-guanosine, 2′-fluoro-guanosine, 7-deaza-guanosine, 8-oxo-guanosine, 2-amino-guanosine, LNA-guanosine, and 2-azido-guanosine.

Without being bound by the theory, it is contemplated that while cap analogs allow for the concomitant capping of a polynucleotide in an in vitro transcription reaction, up to 20% of transcripts remain uncapped. This, as well as the structural differences of a cap analog from the natural 5′-cap structures of polynucleotides produced by the endogenous transcription machinery of a cell, may lead to reduced translational competency and reduced cellular stability.

Accordingly, in some embodiments, a nucleic acid molecule of the present disclosure can also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in connection with the nucleic acid molecules of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, in some embodiments, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap1 structure. This cap results in a higher translational-competency, cellular stability, and a reduced activation of cellular pro-inflammatory cytokines, as compared, e.g., to other 5′cap analog structures known in the art. Other exemplary cap structures include 7mG(5′)ppp(5′)N, pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Without being bound by the theory, it is contemplated that the nucleic acid molecules of the present disclosure can be capped post-transcriptionally, and because this process is more efficient, nearly 100% of the nucleic acid molecules may be capped.

### Untranslated Regions (UTRs)

In some embodiments, the nucleic acid molecules of the present disclosure comprise one or more untranslated regions (UTRs). In some embodiments, an UTR is positioned upstream to a coding region in the nucleic acid molecule, and is termed 5′-UTR. In some embodiments, an UTR is positioned downstream to a coding region in the nucleic acid molecule, and is termed 3′-UTR. The sequence of an UTR can be homologous or heterologous to the sequence of the coding region found in a nucleic acid molecule. Multiple UTRs can be included in a nucleic acid molecule and can be of the same or different sequences, and/or genetic origin. According to the present disclosure, any portion of UTRs in a nucleic acid molecule (including none) can be codon optimized and any may independently contain one or more different structural or chemical modification, before and/or after codon optimization.

In some embodiments, a nucleic acid molecule of the present disclosure (e.g., mRNA) comprises UTRs and coding regions that are homologous with respect to each other. In other embodiments, a nucleic acid molecule of the present disclosure (e.g., mRNA) comprises UTRs and coding regions that are heterogeneous with respect to each other. In some embodiments, to monitor the activity of a UTR sequence, a nucleic acid molecule comprising the UTR and a coding sequence of a detectable probe can be administered in vitro (e.g., cell or tissue culture) or in vivo (e.g., to a subject), and an effect of the UTR sequence (e.g., modulation on the expression level, cellular localization of the encoded product, or half-life of the encoded product) can be measured using methods known in the art.

In some embodiments, the UTR of a nucleic acid molecule of the present disclosure (e.g., mRNA) comprises at least one translation enhancer element (TEE) that functions to increase the amount of polypeptide or protein produced from the nucleic acid molecule. In some embodiments, the TEE is located in the 5′-UTR of the nucleic acid molecule. In other embodiments, the TEE is located at the 3′-UTR of the nucleic acid molecule. In yet other embodiments, at least two TEE are located at the 5′-UTR and 3′-UTR of the nucleic acid molecule respectively. In some embodiments, a nucleic acid molecule of the present disclosure (e.g., mRNA) can comprise one or more copies of a TEE sequence or comprise more than one different TEE sequences. In some embodiments, different TEE sequences that are present in a nucleic acid molecule of the present disclosure can be homologues or heterologous with respect to one another.

Various TEE sequences that are known in the art and can be used in connection with the present disclosure. For example, in some embodiments, the TEE can be an internal ribosome entry site (IRES), HCV-IRES or an IRES element. Chappell et al. Proc. Natl. Acad. Sci. USA 101:9590-9594, 2004; Zhou et al. Proc. Natl. Acad. Sci. 102:6273-6278, 2005. Additional internal ribosome entry site (IRES) that can be used in connection with the present disclosure include but are not limited to those described in U.S. Pat. No. 7,468,275, U.S. Patent Publication No. 2007/0048776 and U.S. Patent Publication No. 2011/0124100 and International Patent Publication No. WO2007/025008 and International Patent Publication No. WO2001/055369, the content of each of which is enclosed herein by reference in its entirety. In some embodiments, the TEE can be those described in Supplemental Table 1 and in Supplemental Table 2 of Wellensiek et al Genome-wide profiling of human cap-independent translation-enhancing elements, Nature Methods, 2013 August; 10(8): 747-750; the content of which is incorporated by reference in its entirety.

Additional exemplary TEEs that can be used in connection with the present disclosure include but are not limited to the TEE sequences disclosed in U.S. Pat. Nos. 6,310,197, 6,849,405, 7,456,273, 7,183,395, U.S. Patent Publication No. 2009/0226470, U.S. Patent Publication No. 2013/0177581, U.S. Patent Publication No. 2007/0048776, U.S. Patent Publication No. 2011/0124100, U.S. Patent Publication No. 2009/0093049, International Patent Publication No. WO2009/075886, International Patent Publication No. WO2012/009644, and International Patent Publication No. WO1999/024595, International Patent Publication No. WO2007/025008, International Patent Publication No. WO2001/055371, European Patent No. 2610341, European Patent No. 2610340, the content of each of which is enclosed herein by reference in its entirety.

In various embodiments, a nucleic acid molecule of the present disclosure (e.g., mRNA) comprises at least one UTR that comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some embodiments, the TEE sequences in the UTR of a nucleic acid molecule are copies of the same TEE sequence. In other embodiments, at least two TEE sequences in the UTR of a nucleic acid molecule are of different TEE sequences. In some embodiments, multiple different TEE sequences are arranged in one or more repeating patterns in the UTR region of a nucleic acid molecule. For illustrating purpose only, a repeating pattern can be, for example, ABABAB, AABBAABBAABB, ABCABCABC, or the like, where in these exemplary patterns, each capitalized letter (A, B, or C) represents a different TEE sequence. In some embodiments, at least two TEE sequences are consecutive with one another (i.e., no spacer sequence in between) in a UTR of a nucleic acid molecule. In other embodiments, at least two TEE sequences are separated by a spacer sequence. In some embodiments, a UTR can comprise a TEE sequence-spacer sequence module that is repeated at least once, at least twice, at least 3 times, at least 4 times, at least 5 times, at least 6 times, at least 7 times, at least 8 times, at least 9 times, or more than 9 times in the UTR. In any of the embodiments described in this paragraph, the UTR can be a 5′-UTR, a 3′-UTR or both 5′-UTR and 3′-UTR of a nucleic acid molecule.

In some embodiments, the UTR of a nucleic acid molecule of the present disclosure (e.g., mRNA) comprises at least one translation suppressing element that functions to decrease the amount of polypeptide or protein produced from the nucleic acid molecule. In some embodiments, the UTR of the nucleic acid molecule comprises one or more miR sequences or fragment thereof (e.g., miR seed sequences) that are recognized by one or more microRNA. In some embodiments, the UTR of the nucleic acid molecule comprises one or more stem-loop structure that downregulates translational activity of the nucleic acid molecule. Other mechanisms for suppressing translational activities associated with a nucleic acid molecules are known in the art. In any of the embodiments described in this paragraph, the UTR can be a 5′-UTR, a 3′-UTR or both 5′-UTR and 3′-UTR of a nucleic acid molecule.

### The Polyadenylation (Poly-A) Regions

During natural RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Without being bound by the theory, it is contemplated that a poly-A region can confer various advantages to the nucleic acid molecule of the present disclosure.

Accordingly, in some embodiments, a nucleic acid molecule of the present disclosure (e.g., an mRNA) comprises a polyadenylation signal. In some embodiments, a nucleic acid molecule of the present disclosure (e.g., an mRNA) comprises one or more polyadenylation (poly-A) regions. In some embodiments, a poly-A region is composed entirely of adenine nucleotides or functional analogs thereof. In some embodiments, the nucleic acid molecule comprises at least one poly-A region at its 3′-end. In some embodiments, the nucleic acid molecule comprises at least one poly-A region at its 5′-end. In some embodiments, the nucleic acid molecule comprises at least one poly-A region at its 5′-end and at least one poly-A region at its 3′-end.

According to the present disclosure, the poly-A region can have varied lengths in different embodiments. Particularly, in some embodiments, the poly-A region of a nucleic acid molecule of the present disclosure is at least 30 nucleotides in length. In some embodiments, the poly-A region of a nucleic acid molecule of the present disclosure is at least 35 nucleotides in length. In some embodiments, the poly-A region of a nucleic acid molecule of the present disclosure is at least 40 nucleotides in length. In some embodiments, the poly-A region of a nucleic acid molecule of the present disclosure is at least 45 nucleotides in length. In some embodiments, the poly-A region of a nucleic acid molecule of the present disclosure is at least 50 nucleotides in length. In some embodiments, the poly-A region of a nucleic acid molecule of the present disclosure is at least 55 nucleotides in length. In some embodiments, the poly-A region of a nucleic acid molecule of the present disclosure is at least 60 nucleotides in length. In some embodiments, the poly-A region of a nucleic acid molecule of the present disclosure is at least 65 nucleotides in length. In some embodiments, the poly-A region of a nucleic acid molecule of the present disclosure is at least 70 nucleotides in length. In some embodiments, the poly-A region of a nucleic acid molecule of the present disclosure is at least 75 nucleotides in length. In some embodiments, the poly-A region of a nucleic acid molecule of the present disclosure is at least 80 nucleotides in length. In some embodiments, the poly-A region of a nucleic acid molecule of the present disclosure is at least 85 nucleotides in length. In some embodiments, the poly-A region of a nucleic acid molecule of the present disclosure is at least 90 nucleotides in length. In some embodiments, the poly-A region of a nucleic acid molecule of the present disclosure is at least 95 nucleotides in length. In some embodiments, the poly-A region of a nucleic acid molecule of the present disclosure is at least 100 nucleotides in length. In some embodiments, the poly-A region of a nucleic acid molecule of the present disclosure is at least 110 nucleotides in length. In some embodiments, the poly-A region of a nucleic acid molecule of the present disclosure is at least 120 nucleotides in length. In some embodiments, the poly-A region of a nucleic acid molecule of the present disclosure is at least 130 nucleotides in length. In some embodiments, the poly-A region of a nucleic acid molecule of the present disclosure is at least 140 nucleotides in length. In some embodiments, the poly-A region of a nucleic acid molecule of the present disclosure is at least 150 nucleotides in length. In some embodiments, the poly-A region of a nucleic acid molecule of the present disclosure is at least 160 nucleotides in length. In some embodiments, the poly-A region of a nucleic acid molecule of the present disclosure is at least 170 nucleotides in length. In some embodiments, the poly-A region of a nucleic acid molecule of the present disclosure is at least 180 nucleotides in length. In some embodiments, the poly-A region of a nucleic acid molecule of the present disclosure is at least 190 nucleotides in length. In some embodiments, the poly-A region of a nucleic acid molecule of the present disclosure is at least 200 nucleotides in length. In some embodiments, the poly-A region of a nucleic acid molecule of the present disclosure is at least 225 nucleotides in length. In some embodiments, the poly-A region of a nucleic acid molecule of the present disclosure is at least 250 nucleotides in length. In some embodiments, the poly-A region of a nucleic acid molecule of the present disclosure is at least 275 nucleotides in length. In some embodiments, the poly-A region of a nucleic acid molecule of the present disclosure is at least 300 nucleotides in length. In some embodiments, the poly-A region of a nucleic acid molecule of the present disclosure is at least 350 nucleotides in length. In some embodiments, the poly-A region of a nucleic acid molecule of the present disclosure is at least 400 nucleotides in length. In some embodiments, the poly-A region of a nucleic acid molecule of the present disclosure is at least 450 nucleotides in length. In some embodiments, the poly-A region of a nucleic acid molecule of the present disclosure is at least 500 nucleotides in length. In some embodiments, the poly-A region of a nucleic acid molecule of the present disclosure is at least 600 nucleotides in length. In some embodiments, the poly-A region of a nucleic acid molecule of the present disclosure is at least 700 nucleotides in length. In some embodiments, the poly-A region of a nucleic acid molecule of the present disclosure is at least 800 nucleotides in length. In some embodiments, the poly-A region of a nucleic acid molecule of the present disclosure is at least 900 nucleotides in length. In some embodiments, the poly-A region of a nucleic acid molecule of the present disclosure is at least 1000 nucleotides in length. In some embodiments, the poly-A region of a nucleic acid molecule of the present disclosure is at least 1100 nucleotides in length. In some embodiments, the poly-A region of a nucleic acid molecule of the present disclosure is at least 1200 nucleotides in length. In some embodiments, the poly-A region of a nucleic acid molecule of the present disclosure is at least 1300 nucleotides in length. In some embodiments, the poly-A region of a nucleic acid molecule of the present disclosure is at least 1400 nucleotides in length. In some embodiments, the poly-A region of a nucleic acid molecule of the present disclosure is at least 1500 nucleotides in length. In some embodiments, the poly-A region of a nucleic acid molecule of the present disclosure is at least 1600 nucleotides in length. In some embodiments, the poly-A region of a nucleic acid molecule of the present disclosure is at least 1700 nucleotides in length. In some embodiments, the poly-A region of a nucleic acid molecule of the present disclosure is at least 1800 nucleotides in length. In some embodiments, the poly-A region of a nucleic acid molecule of the present disclosure is at least 1900 nucleotides in length. In some embodiments, the poly-A region of a nucleic acid molecule of the present disclosure is at least 2000 nucleotides in length. In some embodiments, the poly-A region of a nucleic acid molecule of the present disclosure is at least 2250 nucleotides in length. In some embodiments, the poly-A region of a nucleic acid molecule of the present disclosure is at least 2500 nucleotides in length. In some embodiments, the poly-A region of a nucleic acid molecule of the present disclosure is at least 2750 nucleotides in length. In some embodiments, the poly-A region of a nucleic acid molecule of the present disclosure is at least 3000 nucleotides in length.

In some embodiments, length of a poly-A region in a nucleic acid molecule can be selected based on the overall length of the nucleic acid molecule, or a portion thereof (such as the length of the coding region or the length of an open reading frame of the nucleic acid molecule, etc.). For example, in some embodiments, the poly-A region accounts for about 5%, 10%, 15%, 20%, 25%, 30%, 35%, 40%, 45%, 50%, 55%, 60%, 65%, 70%, 75%, 80%, 85%, 90%, 95% or more of the total length of nucleic acid molecule containing the poly-A region.

Without being bound by the theory, it is contemplated that certain RNA-binding proteins can bind to the poly-A region located at the 3′-end of an mRNA molecule. These poly-A binding proteins (PABP) can modulate mRNA expression, such as interacting with translation initiation machinery in a cell and/or protecting the 3′-poly-A tails from degradation. Accordingly, in some embodiments, in some embodiments, the nucleic acid molecule of the present disclosure (e.g., mRNA) comprises at least one binding site for poly-A binding protein (PABP). In other embodiments, the nucleic acid molecule is conjugated or complex with a PABP before loaded into a delivery vehicle (e.g., lipid nanoparticles).

In some embodiments, the nucleic acid molecule of the present disclosure (e.g., mRNA) comprises a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet structure results in protein production equivalent to at least 75% of that seen using a poly-A region of 120 nucleotides alone.

In some embodiments, the nucleic acid molecule of the present disclosure (e.g., mRNA) may include a poly-A region and may be stabilized by the addition of a 3′-stabilizing region. In some embodiments, the 3′-stabilizing region which may be used to stabilize a nucleic acid molecule (e.g., mRNA) including the poly-A or poly-A-G Quartet structures as described in International Patent Publication No. WO2013/103659, the content of which is incorporated herein by reference in its entirety.

In other embodiments, the 3′-stabilizing region which may be used in connection with the nucleic acid molecules of the present disclosure include a chain termination nucleoside such as but is not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

### Secondary Structure

Without being bound by the theory, it is contemplated that a stem-loop structure can direct RNA folding, protect structural stability of a nucleic acid molecule (e.g., mRNA), provide recognition sites for RNA binding proteins, and serve as a substrate for enzymatic reactions. For example, the incorporation of a miR sequence and/or a TEE sequence changes the shape of the stem loop region which may increase and/or decrease translation (Kedde et al. A Pumilio-induced RNA structure switch in p27-3′UTR controls miR-221 and miR-222 accessibility. Nat Cell Biol., 2010 October; 12(10):1014-20, the content of which is herein incorporated by reference in its entirety).

Accordingly, in some embodiments, the nucleic acid molecules as described herein (e.g., mRNA) or a portion thereof may assume a stem-loop structure, such as but is not limited to a histone stem loop. In some embodiments, the stem-loop structure is formed from a stem-loop sequence that is about 25 or about 26 nucleotides in length such as, but not limited to, those as described in International Patent Publication No. WO2013/103659, the content of which is incorporated herein by reference in its entirety. Additional examples of stem-loop sequences include those described in International Patent Publication No. WO2012/019780 and International Patent Publication No. WO201502667, the contents of which are incorporated herein by reference. In some embodiments, the step-loop sequence comprises a TEE as described herein. In some embodiments, the step-loop sequence comprises a miR sequence as described herein. In specific embodiments, the stem loop sequence may include a miR-122 seed sequence. In specific embodiments, the nucleic acid molecule comprises the stem-loop sequence CAAAGGCTCTTTTCAGAGCCACCA (SEQ ID NO: 1). In other embodiments, the nucleic acid molecule comprises the stem-loop sequence CAAAGGCUCUUUUCAGAGCCACCA (SEQ ID NO:2).

In some embodiments, the nucleic acid molecule of the present disclosure (e.g., mRNA) comprises a stem-loop sequence located upstream (to the 5′-end) of the coding region in a nucleic acid molecule. In some embodiments, the stem-loop sequence is located within the 5′-UTR of the nucleic acid molecule. In some embodiments, the nucleic acid molecule of the present disclosure (e.g., mRNA) comprises a stem-loop sequence located downstream (to the 3′-end) of the coding region in a nucleic acid molecule. In some embodiments, the stem-loop sequence is located within the 3′-UTR of the nucleic acid molecule. In some cases, a nucleic acid molecule can contain more than one stem-loop sequences. In some embodiment, the nucleic acid molecule comprises at least one stem-loop sequence in the 5′-UTR, and at least one stem-loop sequence in the 3′-UTR.

In some embodiments, a nucleic acid molecule comprising a stem-loop structure further comprises a stabilization region. In some embodiment, the stabilization region comprises at least one chain terminating nucleoside that functions to slow down degradation and thus increases the half-life of the nucleic acid molecule. Exemplary chain terminating nucleoside that can be used in connection with the present disclosure include but are not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein. In other embodiments, a stem-loop structure may be stabilized by an alteration to the 3′-region of the polynucleotide that can prevent and/or inhibit the addition of oligio(U) (International Patent Publication No. WO2013/103659, incorporated herein by reference in its entirety).

In some embodiments, a nucleic acid molecule of the present disclosure comprises at least one stem-loop sequence and a poly-A region or polyadenylation signal. Non-limiting examples of polynucleotide sequences comprising at least one stem-loop sequence and a poly-A region or a polyadenylation signal include those described in International Patent Publication No. WO2013/120497, International Patent Publication No. WO2013/120629, International Patent Publication No. WO2013/120500, International Patent Publication No. WO2013/120627, International Patent Publication No. WO2013/120498, International Patent Publication No. WO2013/120626, International Patent Publication No. WO2013/120499 and International Patent Publication No. WO2013/120628, the content of each of which is incorporated herein by reference in its entirety.

In some embodiments, the nucleic acid molecule comprising a stem-loop sequence and a poly-A region or a polyadenylation signal can encode for a pathogen antigen or fragment thereof such as the polynucleotide sequences described in International Patent Publication No. WO2013/120499 and International Patent Publication No. WO2013/120628, the content of each of which is incorporated herein by reference in its entirety.

In some embodiments, the nucleic acid molecule comprising a stem-loop sequence and a poly-A region or a polyadenylation signal can encode for a therapeutic protein such as the polynucleotide sequences described in International Patent Publication No. WO2013/120497 and International Patent Publication No. WO2013/120629, the content of each of which is incorporated herein by reference in its entirety.

In some embodiments, the nucleic acid molecule comprising a stem-loop sequence and a poly-A region or a polyadenylation signal can encode for a tumor antigen or fragment thereof such as the polynucleotide sequences described in International Patent Publication No. WO2013/120500 and International Patent Publication No. WO2013/120627, the content of each of which is incorporated herein by reference in its entirety.

In some embodiments, the nucleic acid molecule comprising a stem-loop sequence and a poly-A region or a polyadenylation signal can code for an allergenic antigen or an autoimmune self-antigen such as the polynucleotide sequences described in International Patent Publication No. WO2013/120498 and International Patent Publication No. WO2013/120626, the content of each of which is incorporated herein by reference in its entirety.

### Functional Nucleotide Analogs

In some embodiments, a payload nucleic acid molecule described herein contains only canonical nucleotides selected from A (adenosine), G (guanosine), C (cytosine), U (uridine), and T (thymidine). Without being bound by the theory, it is contemplated that certain functional nucleotide analogs can confer useful properties to a nucleic acid molecule. Examples of such as useful properties in the context of the present disclosure include but are not limited to increased stability of the nucleic acid molecule, reduced immunogenicity of the nucleic acid molecule in inducing innate immune responses, enhanced production of protein encoded by the nucleic acid molecule, increased intracellular delivery and/or retention of the nucleic acid molecule, and/or reduced cellular toxicity of the nucleic acid molecule, etc.

Accordingly, in some embodiments, a payload nucleic acid molecule comprises at least one functional nucleotide analog as described herein. In some embodiments, the functional nucleotide analog contains at least one chemical modification to the nucleobase, the sugar group and/or the phosphate group. Accordingly, a payload nucleic acid molecule comprising at least one functional nucleotide analog contains at least one chemical modification to the nucleobases, the sugar groups, and/or the internucleoside linkage. Exemplary chemical modifications to the nucleobases, sugar groups, or internucleoside linkages of a nucleic acid molecule are provided herein.

As described herein, ranging from 0% to 100% of all nucleotides in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of all nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

As described herein, ranging from 0% to 100% of all nucleotides of a kind (e.g., all purine-containing nucleotides as a kind, or all pyrimidine-containing nucleotides as a kind, or all A, G, C, T or U as a kind) in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of a kind of nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In some embodiments, a single nucleic acid molecule can contain different sugar modifications, different nucleobase modifications, and/or different types internucleoside linkages (e.g., backbone structures).

### Modification to Nucleobases

In some embodiments, a functional nucleotide analog contains a non-canonical nucleobase. In some embodiments, canonical nucleobases (e.g., adenine, guanine, uracil, thymine, and cytosine) in a nucleotide can be modified or replaced to provide one or more functional analogs of the nucleotide. Exemplary modification to nucleobases include but are not limited to one or more substitutions or modifications including but not limited to alkyl, aryl, halo, oxo, hydroxyl, alkyloxy, and/or thio substitutions; one or more fused or open rings, oxidation, and/or reduction.

In some embodiments, the non-canonical nucleobase is a modified uracil. Exemplary nucleobases and nucleosides having an modified uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s2U), 5-aminomethyl-2-thio-uracil (nm⁵s2U), 5-methylaminomethyl-uracil (mnm⁵U), 5-methylaminomethyl-2-thio-uracil (mnm⁵s2U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s2U), 5-propynyl-uracil, 1-propynyl-pseudouracil, 5-taurinomethyl-uracil (τm ⁵U), 1-taurinomethyl-pseudouridine, 5-taurinomethyl-2-thio-uracil(τm⁵s²U), 1-taurinomethyl-4-thio-pseudouridine, 5-methyl-uracil (m⁵U, i.e., having the nucleobase deoxythymine), 1-methyl-pseudouridine (m¹ψ), 1-ethyl-pseudouridine (Et¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (m⁵U), 5-(isopentenylaminomethyl)-2-thio-uracil (m⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In some embodiments, the non-canonical nucleobase is a modified cytosine. Exemplary nucleobases and nucleosides having a modified cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (fSCm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the non-canonical nucleobase is a modified adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (mlAm), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the non-canonical nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mlG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

In some embodiments, the non-canonical nucleobase of a functional nucleotide analog can be independently a purine, a pyrimidine, a purine or pyrimidine analog. For example, in some embodiments, the non-canonical nucleobase can be modified adenine, cytosine, guanine, uracil, or hypoxanthine. In other embodiments, the non-canonical nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

### Modification to the Sugar

In some embodiments, a functional nucleotide analog contains a non-canonical sugar group. In various embodiments, the non-canonical sugar group can be a 5-carbon or 6-carbon sugar (such as pentose, ribose, arabinose, xylose, glucose, galactose, or a deoxy derivative thereof) with one or more substitutions, such as a halo group, a hydroxy group, a thiol group, an alkyl group, an alkoxy group, an alkenyloxy group, an alkynyloxy group, an cycloalkyl group, an aminoalkoxy group, an alkoxyalkoxy group, an hydroxyalkoxy group, an amino group, an azido group, an aryl group, an aminoalkyl group, an aminoalkenyl group, an aminoalkynyl group, etc.

Generally, RNA molecules contains the ribose sugar group, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In some embodiments, the sugar group contains one or more carbons that possess the opposite stereochemical configuration of the corresponding carbon in ribose. Thus, a nucleic acid molecule can include nucleotides containing, e.g., arabinose or L-ribose, as the sugar. In some embodiments, the nucleic acid molecule includes at least one nucleoside wherein the sugar is L-ribose, 2′-O-methyl-ribose, 2′-fluoro-ribose, arabinose, hexitol, an LNA, or a PNA.

### Modifications to the Internucleoside Linkage

In some embodiments, the payload nucleic acid molecule of the present disclosure can contain one or more modified internucleoside linkage (e.g., phosphate backbone). Backbone phosphate groups can be altered by replacing one or more of the oxygen atoms with a different substituent.

In some embodiments, the functional nucleotide analogs can include the replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy. The replacement of one or more of the oxygen atoms at the position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present disclosure, including internucleoside linkages which do not contain a phosphorous atom, are described herein.

Additional examples of nucleic acid molecules (e.g., mRNA), compositions, formulations and/or methods associated therewith that can be used in connection with the present disclosure further include those described in WO2002/098443, WO2003/051401, WO2008/052770, WO2009127230, WO2006122828, WO2008/083949, WO2010088927, WO2010/037539, WO2004/004743, WO2005/016376, WO2006/024518, WO2007/095976, WO2008/014979, WO2008/077592, WO2009/030481, WO2009/095226, WO2011069586, WO2011026641, WO2011/144358, WO2012019780, WO2012013326, WO2012089338, WO2012113513, WO2012116811, WO2012116810, WO2013113502, WO2013113501, WO2013113736, WO2013143698, WO2013143699, WO2013143700, WO2013/120626, WO2013120627, WO2013120628, WO2013120629, WO2013174409, WO2014127917, WO2015/024669, WO2015/024668, WO2015/024667, WO2015/024665, WO2015/024666, WO2015/024664, WO2015101415, WO2015101414, WO2015024667, WO2015062738, WO2015101416, the content of each of which is incorporated herein in its entirety.

### 5.5 Formulation

According to the present disclosure, nanoparticle compositions described herein can include at least one lipid component and one or more additional components, such as a therapeutic and/or prophylactic agent. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a lipid according to one of formulae (I) (and sub-formulas thereof) described herein, a phospholipid (such as an unsaturated lipid, e.g., DOPE or DSPC), a PEG lipid, and a structural lipid. The elements of the lipid component may be provided in specific fractions.

In one embodiment, provided herein is a nanoparticle compositions comprising a cationic or ionizable lipid compound provided herein, a therapeutic agent, and one or more excipients. In one embodiment, cationic or ionizable lipid compound comprises a compound according to one of Formulae (I) (and sub-formulas thereof) as described herein, and optionally one or more additional ionizable lipid compounds. In one embodiment, the one or more excipients are selected from neutral lipids, steroids, and polymer conjugated lipids. In one embodiment, the therapeutic agent is encapsulated within or associated with the lipid nanoparticle.

In one embodiment, provided herein is a nanoparticle composition (lipid nanoparticle) comprising:
i) between 40 and 50 mol percent of a cationic lipid;
ii) a neutral lipid;
iii) a steroid;
iv) a polymer conjugated lipid; and
v) a therapeutic agent.

As used herein, “mol percent” refers to a component's molar percentage relative to total mols of all lipid components in the LNP (i.e., total mols of cationic lipid(s), the neutral lipid, the steroid and the polymer conjugated lipid).

In one embodiment, the lipid nanoparticle comprises from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, or from 47.2 to 47.8 mol percent of the cationic lipid. In one embodiment, the lipid nanoparticle comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, 7 to 13 mol percent, or 9 to 11 mol percent. In one embodiment, the neutral lipid is present in a concentration of about 9.5, 10 or 10.5 mol percent. In one embodiment, the molar ratio of the cationic lipid to the neutral lipid ranges from about 4.1:1.0 to about 4.9:1.0, from about 4.5:1.0 to about 4.8:1.0, or from about 4.7:1.0 to 4.8:1.0.

In one embodiment, the steroid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent. In one embodiment, the molar ratio of cationic lipid to the steroid ranges from 1.0:0.9 to 1.0:1.2, or from 1.0:1.0 to 1.0:1.2. In one embodiment, the steroid is cholesterol.

In one embodiment, the therapeutic agent to lipid ratio in the LNP (i.e., N/P, were N represents the moles of cationic lipid and P represents the moles of phosphate present as part of the nucleic acid backbone) range from 2:1 to 30:1, for example 3:1 to 22:1. In one embodiment, N/P ranges from 6:1 to 20:1 or 2:1 to 12:1. Exemplary N/P ranges include about 3:1. About 6:1, about 12:1 and about 22:1.

In one embodiment, provided herein is a lipid nanoparticle comprising:
i) a cationic lipid having an effective pKa greater than 6.0; ii) from 5 to 15 mol percent of a neutral lipid;
iii) from 1 to 15 mol percent of an anionic lipid;
iv) from 30 to 45 mol percent of a steroid;
v) a polymer conjugated lipid; and
vi) a therapeutic agent, or a pharmaceutically acceptable salt or prodrug thereof,
wherein the mol percent is determined based on total mol of lipid present in the lipid nanoparticle.

In one embodiment, the cationic lipid can be any of a number of lipid species which carry a net positive charge at a selected pH, such as physiological pH. Exemplary cationic lipids are described herein below. In one embodiment, the cationic lipid has a pKa greater than 6.25. In one embodiment, the cationic lipid has a pKa greater than 6.5. In one embodiment, the cationic lipid has a pKa greater than 6.1, greater than 6.2, greater than 6.3, greater than 6.35, greater than 6.4, greater than 6.45, greater than 6.55, greater than 6.6, greater than 6.65, or greater than 6.7.

In one embodiment, the lipid nanoparticle comprises from 40 to 45 mol percent of the cationic lipid. In one embodiment, the lipid nanoparticle comprises from 45 to 50 mole percent of the cationic lipid.

In one embodiment, the molar ratio of the cationic lipid to the neutral lipid ranges from about 2:1 to about 8:1. In one embodiment, the lipid nanoparticle comprises from 5 to 10 mol percent of the neutral lipid.

Exemplary anionic lipids include, but are not limited to, phosphatidylglycerol, dioleoylphosphatidylglycerol (DOPG), dipalmitoylphosphatidylglycerol (DPPG) or 1,2-distearoyl-sn-glycero-3-phospho-(1′-rac-glycerol) (DSPG).

In one embodiment, the lipid nanoparticle comprises from 1 to 10 mole percent of the anionic lipid. In one embodiment, the lipid nanoparticle comprises from 1 to 5 mole percent of the anionic lipid. In one embodiment, the lipid nanoparticle comprises from 1 to 9 mole percent, from 1 to 8 mole percent, from 1 to 7 mole percent, or from 1 to 6 mole percent of the anionic lipid. In one embodiment, the mol ratio of anionic lipid to neutral lipid ranges from 1:1 to 1:10.

In one embodiment, the steroid cholesterol. In one embodiment, the molar ratio of the cationic lipid to cholesterol ranges from about 5:1 to 1:1. In one embodiment, the lipid nanoparticle comprises from 32 to 40 mol percent of the steroid.

In one embodiment, the sum of the mol percent of neutral lipid and mol percent of anionic lipid ranges from 5 to 15 mol percent. In one embodiment, wherein the sum of the mol percent of neutral lipid and mol percent of anionic lipid ranges from 7 to 12 mol percent.

In one embodiment, the mol ratio of anionic lipid to neutral lipid ranges from 1:1 to 1:10. In one embodiment, the sum of the mol percent of neutral lipid and mol percent steroid ranges from 35 to 45 mol percent.

In one embodiment, the lipid nanoparticle comprises:
i) from 45 to 55 mol percent of the cationic lipid;
ii) from 5 to 10 mol percent of the neutral lipid;
iii) from 1 to 5 mol percent of the anionic lipid; and
iv) from 32 to 40 mol percent of the steroid.

In one embodiment, the lipid nanoparticle comprises from 1.0 to 2.5 mol percent of the conjugated lipid. In one embodiment, the polymer conjugated lipid is present in a concentration of about 1.5 mol percent.

In one embodiment, the neutral lipid is present in a concentration ranging from 5 to 15 mol percent, 7 to 13 mol percent, or 9 to 11 mol percent. In one embodiment, the neutral lipid is present in a concentration of about 9.5, 10 or 10.5 mol percent. In one embodiment, the molar ratio of the cationic lipid to the neutral lipid ranges from about 4.1:1.0 to about 4.9:1.0, from about 4.5:1.0 to about 4.8:1.0, or from about 4.7:1.0 to 4.8:1.0.

In one embodiment, the steroid is cholesterol. In some embodiments, the steroid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In one embodiment, the steroid is present in a concentration of 40, 41, 42, 43, 44, 45, or 46 molar percent. In certain embodiments, the molar ratio of cationic lipid to the steroid ranges from 1.0:0.9 to 1.0:1.2, or from 1.0:1.0 to 1.0:1.2.

In one embodiment, the molar ratio of cationic lipid to steroid ranges from 5:1 to 1:1.

In one embodiment, the lipid nanoparticle comprises from 1.0 to 2.5 mol percent of the conjugated lipid. In one embodiment, the polymer conjugated lipid is present in a concentration of about 1.5 mol percent.

In one embodiment, the molar ratio of cationic lipid to polymer conjugated lipid ranges from about 100:1 to about 20:1. In one embodiment, the molar ratio of cationic lipid to the polymer conjugated lipid ranges from about 35:1 to about 25:1.

In one embodiment, the lipid nanoparticle has a mean diameter ranging from 50 nm to 100 nm, or from 60 nm to 85 nm.

In one embodiment, the composition comprises a cationic lipid provided herein, DSPC, cholesterol, and PEG-lipid, and mRNA. In one embodiment, the a cationic lipid provided herein, DSPC, cholesterol, and PEG-lipid are at a molar ratio of about 50:10:38.5:1.5.

Nanoparticle compositions can be designed for one or more specific applications or targets. For example, a nanoparticle composition can be designed to deliver a therapeutic and/or prophylactic agent such as an RNA to a particular cell, tissue, organ, or system or group thereof in a mammal's body. Physiochemical properties of nanoparticle compositions can be altered in order to increase selectivity for particular bodily targets. For instance, particle sizes can be adjusted based on the fenestration sizes of different organs. The therapeutic and/or prophylactic agent included in a nanoparticle composition can also be selected based on the desired delivery target or targets. For example, a therapeutic and/or prophylactic agent can be selected for a particular indication, condition, disease, or disorder and/or for delivery to a particular cell, tissue, organ, or system or group thereof (e.g., localized or specific delivery). In certain embodiments, a nanoparticle composition can include an mRNA encoding a polypeptide of interest capable of being translated within a cell to produce the polypeptide of interest. Such a composition can be designed to be specifically delivered to a particular organ. In certain embodiments, a composition can be designed to be specifically delivered to a mammalian liver.

The amount of a therapeutic and/or prophylactic agent in a nanoparticle composition can depend on the size, composition, desired target and/or application, or other properties of the nanoparticle composition as well as on the properties of the therapeutic and/or prophylactic agent. For example, the amount of an RNA useful in a nanoparticle composition can depend on the size, sequence, and other characteristics of the RNA. The relative amounts of a therapeutic and/or prophylactic agent and other elements (e.g., lipids) in a nanoparticle composition can also vary. In some embodiments, the wt/wt ratio of the lipid component to a therapeutic and/or prophylactic agent in a nanoparticle composition can be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic agent can be from about 10:1 to about 40:1. In certain embodiments, the wt/wt ratio is about 20:1. The amount of a therapeutic and/or prophylactic agent in a nanoparticle composition can, for example, be measured using absorption spectroscopy (e.g., ultraviolet-visible spectroscopy).

In some embodiments, a nanoparticle composition includes one or more RNAs, and the one or more RNAs, lipids, and amounts thereof can be selected to provide a specific N:P ratio. The N:P ratio of the composition refers to the molar ratio of nitrogen atoms in one or more lipids to the number of phosphate groups in an RNA. In some embodiments, a lower N:P ratio is selected. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

The physical properties of a nanoparticle composition can depend on the components thereof. For example, a nanoparticle composition including cholesterol as a structural lipid can have different characteristics compared to a nanoparticle composition that includes a different structural lipid. Similarly, the characteristics of a nanoparticle composition can depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

In various embodiments, the mean size of a nanoparticle composition can be between 10s of nm and 100s of nm. For example, the mean size can be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition can be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition can be from about 70 nm to about 100 nm. In some embodiments, the mean size can be about 80 nm. In other embodiments, the mean size can be about 100 nm.

A nanoparticle composition can be relatively homogenous. A polydispersity index can be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition can be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic agent describes the amount of therapeutic and/or prophylactic agent that is encapsulated or otherwise associated with a nanoparticle composition after preparation, relative to the initial amount provided. The encapsulation efficiency is desirably high (e.g., close to 100%). The encapsulation efficiency can be measured, for example, by comparing the amount of therapeutic and/or prophylactic agent in a solution containing the nanoparticle composition before and after breaking up the nanoparticle composition with one or more organic solvents or detergents. Fluorescence can be used to measure the amount of free therapeutic and/or prophylactic agent (e.g., RNA) in a solution. For the nanoparticle compositions described herein, the encapsulation efficiency of a therapeutic and/or prophylactic agent can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

A nanoparticle composition can optionally comprise one or more coatings. For example, a nanoparticle composition can be formulated in a capsule, film, or tablet having a coating. A capsule, film, or tablet including a composition described herein can have any useful size, tensile strength, hardness, or density.

### 5.6 Pharmaceutical Compositions

According to the present disclosure, nanoparticle compositions can be formulated in whole or in part as pharmaceutical compositions. Pharmaceutical compositions can include one or more nanoparticle compositions. For example, a pharmaceutical composition can include one or more nanoparticle compositions including one or more different therapeutic and/or prophylactic agents. Pharmaceutical compositions can further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients can be used in any pharmaceutical composition, except insofar as any conventional excipient or accessory ingredient can be incompatible with one or more components of a nanoparticle composition. An excipient or accessory ingredient can be incompatible with a component of a nanoparticle composition if its combination with the component can result in any undesirable biological effect or otherwise deleterious effect.

In some embodiments, one or more excipients or accessory ingredients can make up greater than 50% of the total mass or volume of a pharmaceutical composition including a nanoparticle composition. For example, the one or more excipients or accessory ingredients can make up 50%, 60%, 70%, 80%, 90%, or more of a pharmaceutical composition. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition can comprise between 0.1% and 100% (wt/wt) of one or more nanoparticle compositions.

In certain embodiments, the nanoparticle compositions and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising a compound of any of Formulae (I) (and sub-formulas thereof) is a solution that is refrigerated for storage and/or shipment at, for example, about −20° C., −30° C., −40° C., −50° C., −60° C., −70° C., or −80° C. In certain embodiments, the disclosure also relates to a method of increasing stability of the nanoparticle compositions and/or pharmaceutical compositions comprising a compound of any of Formulae (I) (and sub-formulas thereof) by storing the nanoparticle compositions and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.). For example, the nanoparticle compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In one embodiment, the formulation is stabilized for at least 4 weeks at about 4° C. In certain embodiments, the pharmaceutical composition of the disclosure comprises a nanoparticle composition disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8, 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a nanoparticle composition disclosed herein, Tris, saline and sucrose, and has a pH of about 7.5-8, which is suitable for storage and/or shipment at, for example, about −20° C. For example, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

Nanoparticle compositions and/or pharmaceutical compositions including one or more nanoparticle compositions can be administered to any patient or subject, including those patients or subjects that can benefit from a therapeutic effect provided by the delivery of a therapeutic and/or prophylactic agent to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, horses, sheep, cats, dogs, mice, and/or rats.

A pharmaceutical composition including one or more nanoparticle compositions can be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions can be prepared in a variety of forms suitable for a variety of routes and methods of administration. For example, pharmaceutical compositions can be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactic agents, additional agents such as wetting agents, emulsifying and suspending agents, sweetening, flavoring, and/or perfuming agents. In certain embodiments for parenteral administration, compositions are mixed with solubilizing agents such as Cremophor, alcohols, oils, modified oils, glycols, polysorbates, cyclodextrins, polymers, and/or combinations thereof.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

The disclosure features methods of delivering a therapeutic and/or prophylactic agent to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic agent.

### 6. EXAMPLES

The examples in this section are offered by way of illustration, and not by way of limitation.

### General Methods.

General preparative HPLC method: HPLC purification is carried out on an Waters 2767 equipped with a diode array detector (DAD) on an Inertsil Pre-C8 OBD column, generally with water containing 0.1% TFA as solvent A and acetonitrile as solvent B.

General LCMS method: LCMS analysis is conducted on a Shimadzu (LC-MS2020) System. Chromatography is performed on a SunFire C18, generally with water containing 0.1% formic acid as solvent A and acetonitrile containing 0.1% formic acid as solvent B.

### 6.1 Example 1: Preparation of Starting Materials and Intermediates

### Preparation of Compound A

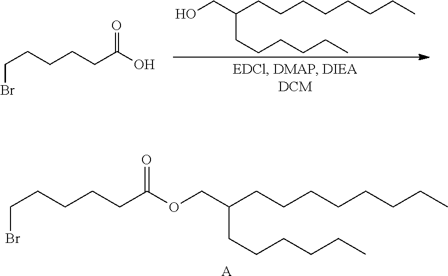

To a solution of 2-hexyldecan-1-ol (2.0 g, 8.33 mmol, 1.0 eq.) and 6-bromohexanoic acid (2.0 g, 10.0 mmol, 1.2 eq.) in 30 mL of dichloromethane was added diisopropylethylamine (2.7 g, 2.08 mmol, 2.5 eq.) and DMAP (203 mg, 1.67 mmol, 0.2 eq.). After stirring for 5 min at ambient temperature, EDCI (2.4 g, 12.5 mmol, 1.5 eq.) was added and the reaction mixture was stirred at room temperature overnight after which the TLC showed complete disappearance of the starting alcohol. The reaction mixture was diluted with CH₂Cl₂ (300 mL) and washed with saturated NaHCO₃ (100 mL), water (100 mL) and brine (100 mL). The combined organic layers were dried over Na₂SO₄ and solvents were removed in vacuo. Evaporation of the solvent provided the crude product which was purified by column (silica gel, 0-1% ethyl acetate (EA) in hexane) chromatography to provide compound A (2.0 g, 57%) as a colorless oil.

### Preparation of Compound B

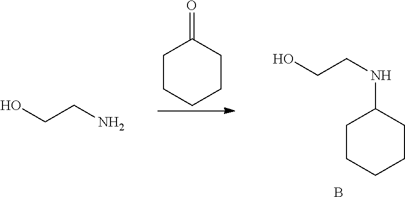

A mixture of the cyclohexanone (2.0 g, 20.0 mmol, 1.0 eq.), titanium (IV) isopropoxide (7.4 g, 26 mmol, 1.3 eq.), and 2-aminoethanol (3.66 g, 60.0 mmol, 3.0 eq.) in methanol (10.0 mL) was stirred under argon at RT for 5 hours. Sodium borohydride (760.0 mg, 20.0 mmol, 1.0 eq.) was then added at 0° C. and the resulting mixture was stirred for an additional 2 h. The reaction was then quenched by adding water (10.0 mL). Stirring was continued at RT for 20 min then the reaction mixture was acidified with hydrochloric acid (1 M, 5 mL), filtered over a pad of Celite, and washed with water and EA. The organic layer was separated and dried over Na₂SO₄, evaporated under reduced pressured and purified with flash column chromatography (FCC) (PE/EA=5/1-0/1) to provide compound B (1.5 g, 52% yield) as a yellow oil.

### Preparation of Compound C

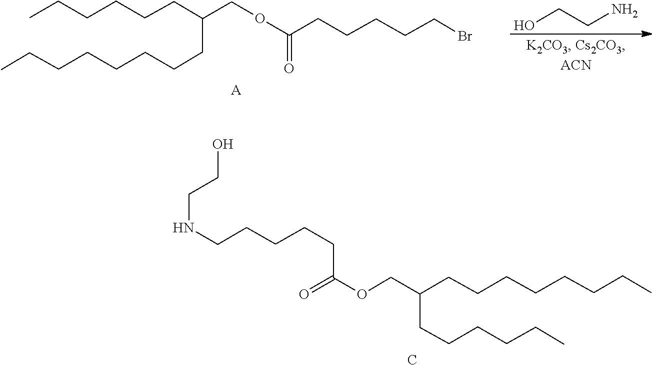

To a solution of compound A (446.0 mg, 1.0 mmol, 1.0 eq.) and ethanolamine (180.0 mg, 3.0 mmol, 3.0 eq.) in acetonitrile (ACN, 10.0 mL) was added Cs₂CO₃ (97.5 mg, 0.3 mmol, 0.3 eq.), K₂CO₃ (414.0 mg, 3.0 mmol, 3.0 eq.) and NaI (14.6 mg, 0.1 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound C (0.35 g, 82% yield) as a yellow oil.

### Preparation of Compound D

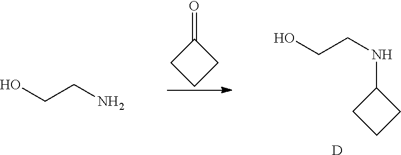

A mixture of cyclobutanone (8.0 g, 114 mol, 1.0 eq.) and 2-aminoethanol (20.9 g, 342 mol, 3.0 eq.) in methanol (100 mL) was stirred under argon at RT for 16 hours. Sodium borohydride (4.3 g, 114 mmol, 1.0 eq.) was then added at 0° C. and the resulting mixture was stirred for an additional 16 h. The reaction mixture was then concentrated under reduced pressure. Added water (200 mL) and extracted with dichloromethane (DCM). The combined organic layers were dried over Na₂SO₄ and evaporated under reduced pressured and purified by column chromatography (silica gel, 2%-10% MeOH in DCM) to provide compound D (3.9 g, 30% yield) as light yellow oil.

### Preparation of Compound E

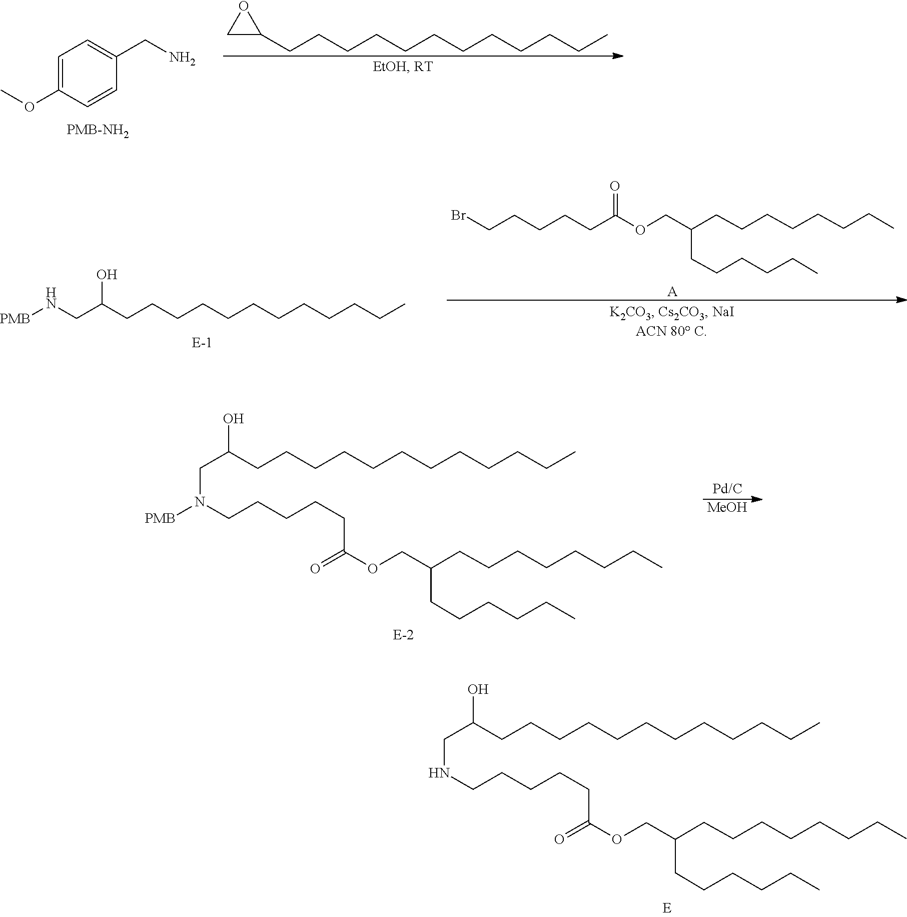

### Step 1: Preparation of Compound E-1

To a solution of PMB-NH₂ (5.166 g, 37.66 mmol, 4.0 eq.) in EtOH (30 mL) was added 1,2-epoxytetradecane (2.0 g, 9.416 mmol, 1.0 eq.). The reaction mixture was stirred at RT for 16 hours. LCMS showed the reaction was complete. The mixture was evaporated under reduced pressure and purified by FCC to provide compound E-1 (1.42 g, 43.09%) as white solid. LCMS: Rt: 0.815 min; MS m/z (ESI): 350.3 [M+H]⁺.

### Step 2: Preparation of Compound E-2

To a solution of compound E-1 (1.42 g, 4.057 mmol, 1.0 eq.) in ACN (25 mL) was added compound A (5.106 mg, 12.17 mmol, 3.0 eq.), K₂CO₃ (1.668 g, 12.17 mmol, 3.0 eq.), Cs₂CO₃ (397 mg, 1.217 mmol, 0.3 eq.), and NaI (30 mg, 0.2029 mmol, 0.05 eq.). The reaction mixture was stirred at 80° C. for 16 hours. LCMS showed the reaction was complete. Removal of solvent and purification by FCC provided compound E-2 (2.5 g, 89.55%) as colorless oil. LCMS: Rt: 0.241 min; MS m/z (ESI): 688.5 [M+H]⁺.

### Step 3: Preparation of Compound E

To a solution of compound E-2 (250 mg, 0.3633 mmol) in MeOH (10 mL) was added Pd/C (50 mg). The reaction mixture was stirred at RT for 16 hours under H₂. LCMS showed the reaction was complete. After removal of solvent, purification by pre-HPLC provided compound E (105 mg, 50.88% yield) as colorless oil.

¹H NMR (400 MHz, CDCl₃): 3.97 (d, J=6 Hz, 2H), 3.58 (s, 1H), 2.73-2.58 (m, 3H), 2.45-2.40 (m, 1H), 2.33-2.29 (m, 2H), 1.66-1.60 (m, 2H), 1.51-1.40 (m, 2H), 1.39-1.34 (m, 4H), 1.26 (s, 46H), 0.90-0.86 (m, 9H). LCMS: Rt: 1.083 min; MS m/z (ESI): 568.5 [M+H]⁺.

### Preparation of Compound F

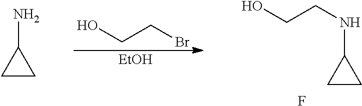

To a mixture of cyclopropanamine (5.7 g, 100 mmol, 2.5 eq.) in EtOH (50 mL) was added 2-bromoethanol (5 g, 40 mmol, 1 eq.). The reaction mixture was stirred at 50° C. for 16 hours. LCMS showed the reaction was complete. Removal of solvent provided compound F (6.6 g crude) as a yellow oil.

### Preparation of Compound G

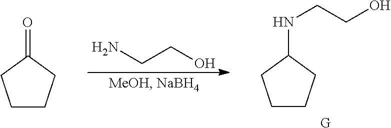

A solution of cyclopentanone (16.8 g, 200 mmol, 1 eq.) and 2-aminoethanol (13.4 g, 220 mmol, 1.1 eq.) with 3 drop of acetic acid (AcOH) in MeOH (300 mL) was stirred overnight at room temperature, and then NaBH₄ (8.4 g, 220 mmol, 1.1 eq.) was added to the mixture at 0° C. The mixture was stirred for two hours at RT. The mixture was quenched with water (100 mL), extracted with EA (3×100 mL), dried, and concentrated. Purification by silica gel column chromatography (MeOH:DCM=0% to 10%) provided compound G (17.8 g, 49.0% yield) as a yellow oil.

### Preparation of Compound H

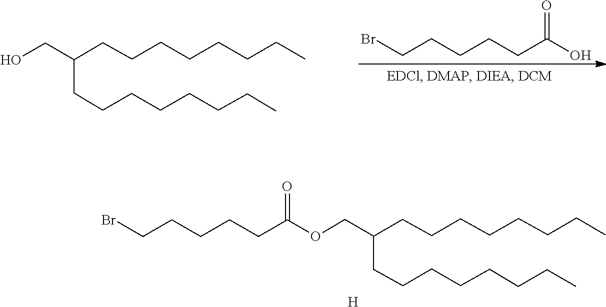

To a solution of 2-octyldecan-1-ol (1.5 g, 5.545 mmol, 1.0 eq.) in DCM (15 mL) was added 6-bromohexanoic acid (1.3 g, 6.654 mmol, 1.2 eq.), EDCI (1.6 g, 8.318 mmol, 1.5 eq.), DMAP (135 mg, 1.109 mmol, 0.2 eq.), and diisopropylethylamine (DIEA, 1.4 g, 11.09 mmol, 2.0 eq.). The reaction mixture was stirred at 50° C. for 16 hours. TLC showed the reaction was complete. Removal of solvent and purification of the crude product by FCC provided compound H (1.2 g, 48.36%) as a yellow oil.

### Preparation of Compound K

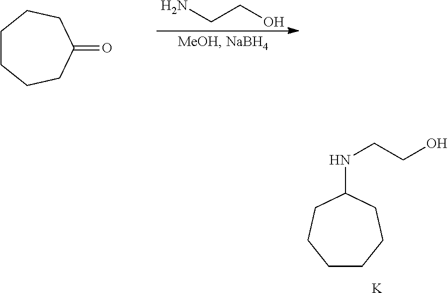

A mixture of cycloheptanone (15 g, 134 mmol, 1 eq.) and 2-aminoethanol (9 g, 147 mmol, 1.1 eq.) with 3 drop of AcOH in MeOH (250 mL) was stirred overnight at room temperature, and then NaBH₄ (5.6 g, 147 mmol, 1.1 eq.) was added to the mixture at 0° C. The mixture was stirred for two hours at RT. The mixture was quenched with water (100 mL), extracted with EA (3×100 mL), dried, and concentrated. Purification by silica gel column chromatography (MeOH:DCM=0% to 10%) provided compound K (10.3 g, 69.2% yield) as a yellow oil.

### Preparation of Compound L

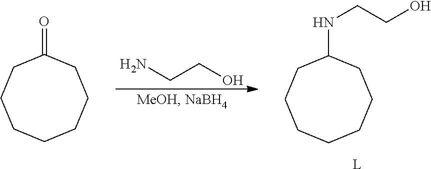

A mixture of cyclooctanone (2.0 g, 15.85 mmol, 1 eq.) and 2-aminoethanol (1.07 g, 17.43 mmol, 1.1 eq.) with 3 drop of AcOH in MeOH (30 ml) was stirred overnight at room temperature, and then NaBH₄ (660 mg, 17.43 mmol, 1.1 eq.) was added to the mixture at 0° C. The mixture was stirred for two hours at room temperature. The mixture was quenched with water (100 mL), extracted over EA (3×100 mL) and dried. After concentrated, the residual was purified by column chromatography silica gel (MeOH:DCM=0% to 10%) to provide compound L (960 mg, 35% yield) as yellow oil.

### Preparation of SM2:

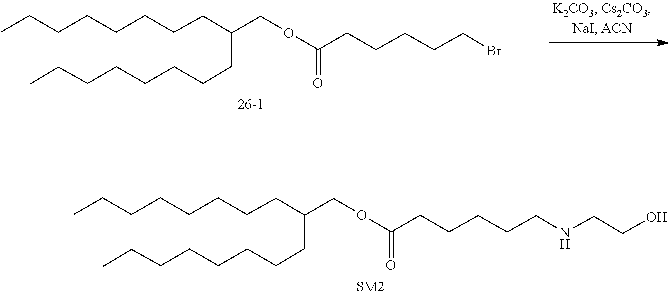

A mixture of compound 26-1 (250 mg, 0.56 mmol, 1.0 eq.), 2-aminoethanol (243 mg, 1.68 mmol, 3.0 eq.), K₂CO₃ (232 mg, 1.68 mmol, 3.0 eq.), Cs₂CO₃ (7 mg, 0.02 mmol, 0.03 eq.) and sodium iodide (30 mg, 0.2 mmol, 0.3 eq.) in ACN (10 mL) was stirred overnight at 100° C. The mixture was concentrated and purified by column chromatography silica gel (MeOH:DCM=0% to 10%) to give the desired product SM2 (1.78 g, 62.1% yield) as yellow oil. LCMS: Rt: 1.427 min; MS m/z (ESI): 428.5 [M+H]⁺.

### Preparation of SM4:

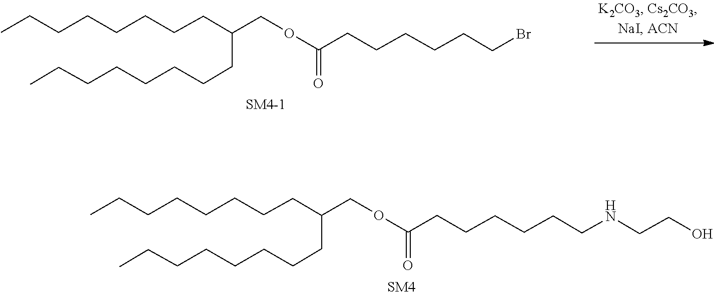

A mixture of compound SM4-1 (2.1 g, 4.5 mmol, 1.0 eq.), 2-aminoethanol (830 mg, 13.6 mmol, 3.0 eq.), K₂CO₃ (1.9 g, 13.6 mmol, 3.0 eq.), Cs₂CO₃ (440 mg, 1.4 mmol, 0.3 eq.), NaI (200 mg, 1.4 mmol, 0.3 eq.) in ACN (15 mL) was stirred overnight at reflux. The mixture was diluted with water, extracted with EA, concentrated and purified by column chromatography silica gel (MeOH:DCM=0% to 10%) to give the desired product SM4 (860 mg, 41% yield) as yellow oil. LCMS: Rt: 1.000 min; MS m/z (ESI): 442.4 [M+H]⁺.

### Preparation of SM9:

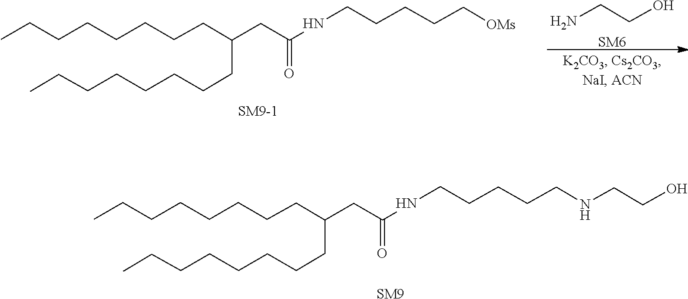

To a solution of compound SM9-1 (1.0 g, 2.166 mmol, 1.0 eq.) in ACN (15 mL) was added compound SM6 (0.4 g, 6.498 mmol, 3.0 eq.), K₂CO₃ (0.9 g, 6.498 mmol, 3.0 eq.), Cs₂CO₃ (212 mg, 0.6498 mmol, 0.3 eq.), NaI (32 mg, 0.2166 mmol, 0.1 eq.). The reaction mixture was stirred at 80° C. for 16 hours. LCMS showed the reaction was complete. Removal of solvent, FCC to get the compound SM9 (350 mg, 37.87%) as yellow oil.

### Preparation of SM10:

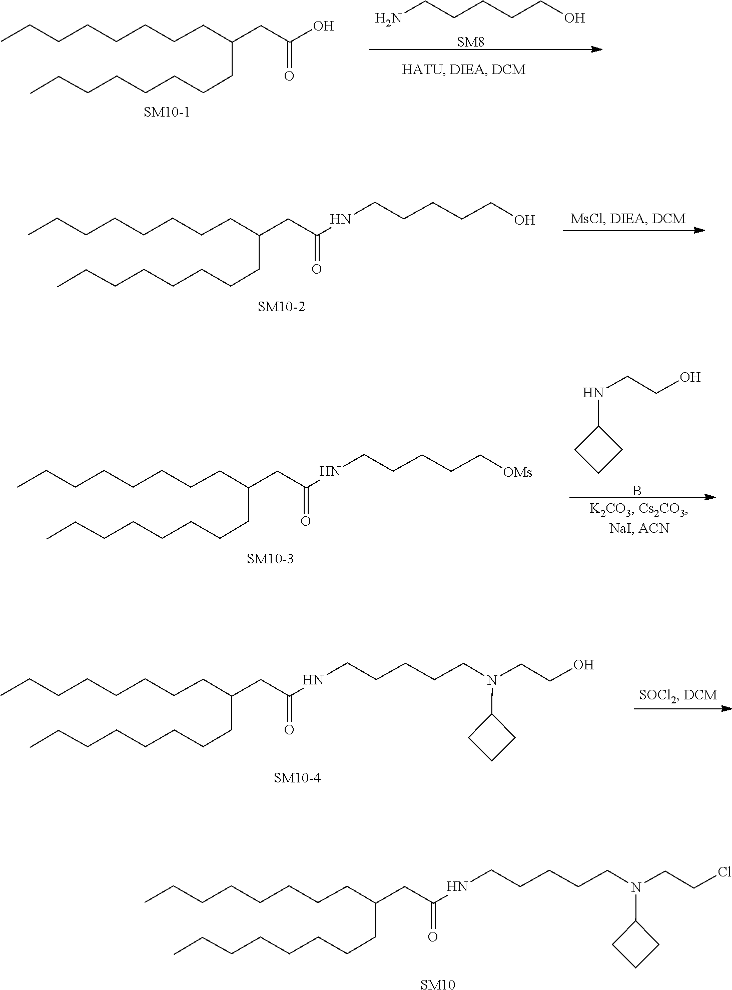

### Step 1: Preparation of Compound SM10-2

To a mixture of compound SM10-1 (2.0 g, 6.700 mmol, 1.0 eq.), compound SM8 (0.83 g, 8.040 mmol, 1.2 eq.), DIEA (2.6 g, 20.10 mmol, 3.0 eq.) in DCM (30 mL) was added HATU (3.8 g, 10.50 mmol, 1.5 eq.). The reaction mixture was stirred at rt for 1 hours. TLC showed the reaction was complete. The mixture power in water and washed with DCM. The organic was separated and dried over Na₂SO₄. Removal of solvent, FCC to get the compound SM10-2 (2.4 g, 93.36%) as colorless oil.

### Step 2: Preparation of Compound SM10-3

To a mixture of compound SM10-2 (2.4 g, 6.255 mmol, 1.0 eq.), DIEA (1.62 g, 12.51 mmol, 2.0 eq.) in DCM (60 mL) was added MsCl (0.86 g, 7.506 mmol, 1.2 eq.) at 0° C. under N₂. The reaction mixture was stirred at 0° C. for 1 hours. TLC showed the reaction was complete. The mixture was poured in water and washed with DCM. The organic was separated and dried over Na₂SO₄. Removal of solvent, FCC to get the compound SM10-3 (2.5 g, 86.57%) as yellow oil.

### Step 3: Preparation of Compound SM10-4

To a solution of compound SM10-3 (1.5 g, 3.249 mmol, 1.0 eq.) in ACN (30 mL) was added compound B (0.45 g, 3.899 mmol, 1.2 eq.), K₂CO₃ (1.35 g, 9.747 mmol, 3.0 eq.), Cs₂CO₃ (318 mg, 0.9747 mmol, 0.3 eq.), NaI (49 mg, 0.3249 mmol, 0.1 eq.). The reaction mixture was stirred at 80° C. for 16 hours. LCMS showed the reaction was complete. Removal of solvent, FCC to get the compound SM10-4 (700 mg, 44.81%) as yellow oil. LCMS: Rt: 0.830 min; MS m/z (ESI): 481.4 [M+H]⁺.

### Step 4: Preparation of Compound SM10

To a solution of compound SM10-4 (300 mg, 0.6240 mmol, 1.0 eq.) in DCM (15 mL) was added SOCl₂ (223 mg, 1.872 mmol, 3.0 eq.). The reaction mixture was stirred at 35° C. for 16 hours. LCMS showed the reaction was complete. Removal of solvent to get the compound SM10 (310 mg, crude) as yellow oil. LCMS: Rt: 0.860 min; MS m/z (ESI): 499.3 [M+H]⁺.

### Preparation of SM11:

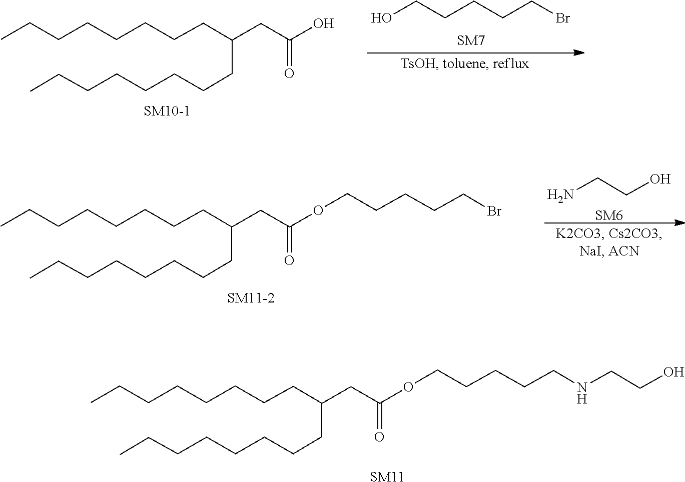

### Step 1: Preparation of Compound SM11-2

A mixture of compound SM10-1 (1.5 g, 5.025 mmol, 1.0 eq.), compound SM7 (1.26 g, 7.538 mmol, 1.5 eq.), TsOH (300 mg) in toluene (20 mL) was stirred at reflux for 2 hours. TLC showed the reaction was complete. The mixture was evaporated under reduced pressure and FCC to get the compound SM11-2 (1.4 g, 62.26%) as yellow oil.

### Step 2: Preparation of Compound SM11

To a solution of compound SM11-2 (1.0 g, 2.235 mmol, 1.0 eq.) in ACN (15 mL) were added compound SM6 (0.41 g, 6.704 mmol, 3.0 eq.), K₂CO₃ (0.93 g, 6.704 mmol, 3.0 eq.), Cs₂CO₃ (218 mg, 0.6704 mmol, 0.3 eq.), NaI (33 mg, 0.2235 mmol, 0.1 eq.). The reaction mixture was stirred at 80° C. for 16 hours. LCMS showed the reaction was complete. Removal of solvent, FCC to get the compound SM11 (700 mg, 44.81%) as yellow oil. LCMS: Rt: 0.890 min; MS m/z (ESI): 428.3 [M+H]⁺.

### Preparation of SM:

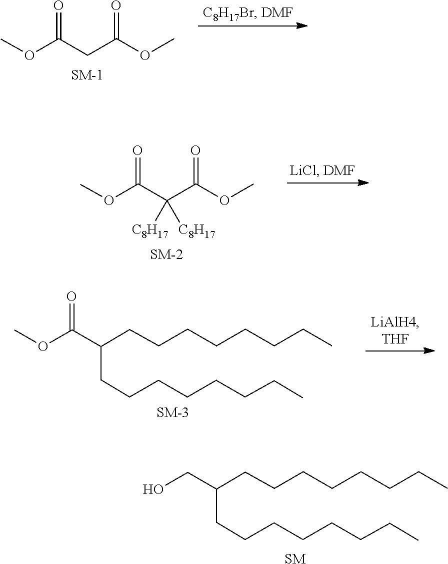

### Step 1: Preparation of Compound SM-2

To a mixture of NaH (12 g, 227.1 mmol, 2.5 eq.) in DMF (100 mL) was added compound SM-1 (12 g, 90.84 mmol, 1.0 eq.) at 0° C. under N₂. The reaction mixture was stirred at 0° C. for 1 hours. The C₈H₁₇Br (44 g, 227.1 mmol, 2.5 eq.) in DMF (100 mL) was added to it. The reaction mixture was stirred at rt for 16 hours. TLC showed the reaction was complete. The mixture power in water and washed with EA. The organic was separated and dried over Na₂SO₄. Removal of solvent, FCC to get the compound SM-2 (17.8 g, 54.96%) as colorless oil. ¹H NMR (400 MHz, CCl₃D): 3.71 (s, 6H), 1.88-1.84 (m, 4H), 1.59 (s, 1H), 1.25 (s, 19H), 1.14-1.10 (m, 4H), 0.89-0.86 (m, 6H).

### Step 2: Preparation of Compound SM-3

To a solution of SM-2 (17.8 g, 49.93 mmol, 1.0 eq.) in DMF (260 mL) was added LiCl (21.17 g, 499.3 mmol, 10.0 eq.). The reaction mixture was stirred at 120° C. for 12 hours. TLC showed the reaction was complete. The mixture power in water and washed with EA. The organic was separated and dried over Na₂SO₄. Removal of solvent, FCC to get the compound SM-3 (10 g, 67.10%) as colorless oil. ¹H NMR (400 MHz, CCl₃D): 0.89-0.86 (m, 6H), 1.25 (s, 22H), 1.45-1.40 (m, 2H), 1.59 (s, 4H), 2.36-2.30 (m, 1H), 3.67 (s, 3H).

### Step 3: Preparation of Compound SM

To a solution of compound SM-3 (10 g, 33.50 mmol, 1.0 eq.) in THF (100 mL) was added slowly LiAlH₄ (2.546 g, 67.00 mmol, 2.0 eq.) in 0° C. The reaction mixture was stirred at reflux for 1 hour. TLC showed the reaction was complete. After being cooled to 0° C., the mixture was quenched with successive addition of water (3.4 mL), 15% aq. NaOH (3.4 mL) and water (10 mL). The resulting mixture was diluted with EA and the precipitate was removed by filtration. The filtrate was evaporated under reduced pressure and FCC to get the compound SM (8.5 g, 93.80%) as yellow oil. ¹H NMR (400 MHz, CCl₃D): 0.90-0.86 (m, 6H), 1.27 (s, 27H), 1.43 (s, 3H), 3.54 (d, J=5.2 Hz, 2H).

### Preparation of SM15:

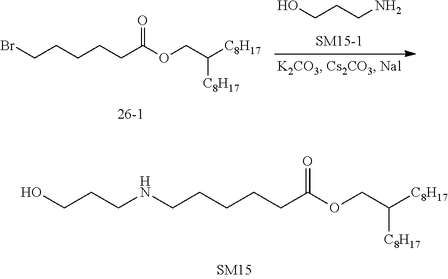

To a solution of compound 26-1 (400 mg, 0.89 mmol, 1.0 eq.) in ACN (30 mL) was added compound SM15-1 (140 mg, 1.79 mmol, 2.0 eq.), K₂CO₃ (370 mg, 2.68 mmol, 3.0 eq.), Cs₂CO₃ (90 mg, 0.27 mmol, 0.3 eq.) and NaI (40 mg, 0.27 mmol, 0.3 eq.). The reaction mixture was stirred at 80° C. for 10 hours. LCMS showed the reaction was complete. Removal of solvent, FCC to get the compound SM15 (120 mg, 30%). LCMS: Rt: 0.900 min; MS m/z (ESI): 442.3 [M+H]⁺.

### Preparation of SM16:

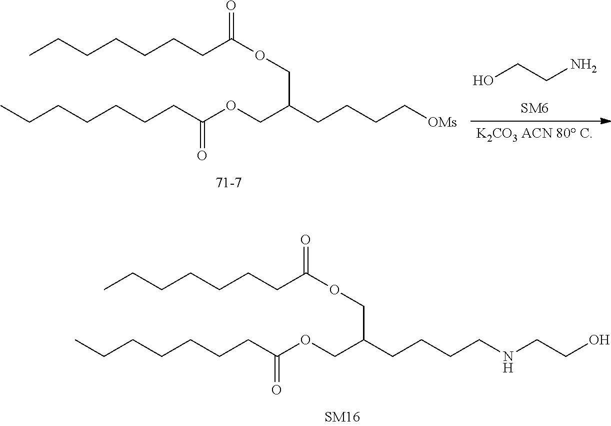

To a solution of compound 71-7 (420 mg, 0.88 mmol, 1.0 eq.) and compound SM6 (108 mg, 1.76 mmol, 2.0 eq.) in ACN (20 mL) were added K₂CO₃ (365 mg, 2.64 mmol, 3.0 eq.), Cs₂CO₃ (85 mg, 0.26 mmol, 0.3 eq.) and NaI (39 mg, 0.26 mmol, 0.3 eq.). The mixture was stirred at 80° C. for 16 hours. LCMS showed the reaction was complete. The reaction mixture was concentrated and purified by column chromatography on silica gel (DCM/MeOH=10/1) to give compound SM16 (146 mg, 37% yield) as yellow oil. LCMS: Rt: 0.810 min; MS m/z (ESI): 444.3 [M+H]⁺.

### Preparation of SM18:

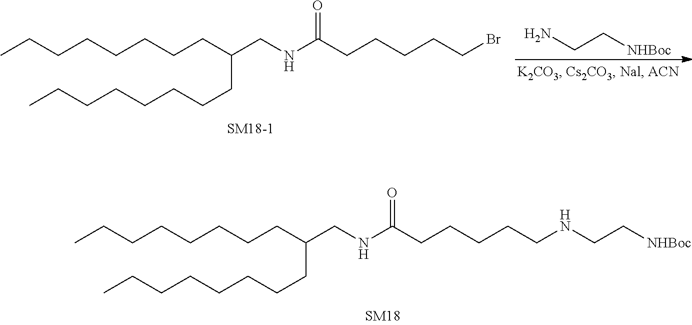 [SMILES: CCCCCCCCC(CCCCCCCC)CNC(=O)CCCCCBr ; CC(C)(C)OC(=O)NCCN ; CCCCCCCCC(CCCCCCCC)CNC(=O)CCCCCNCCNC(=O)OC(C)(C)C]

A mixture of compound SM18-1 (2.0 g, 4.48 mmol, 1.0 eq.), tert-butyl (2-aminoethyl) carbamate (1.0 g, 6.72 mmol, 1.5 eq.), K₂CO₃ (1.8 g, 13.4 mmol, 3.0 eq.), Cs₂CO₃ (440 mg, 1.34 mmol, 0.3 eq.), NaI (200 mg, 1.34 mmol, 0.3 eq.) in ACN (20 mL) was stirred at 90° C. for overnight. LCMS showed the target product. The mixture was concentrated and the residue was purified by column chromatography to give product SM18 (860 mg, 36.5% yield) as white solid. LCMS: Rt: 0.870 min; MS m/z (ESI): 526.5 [M+H]⁺.

### Preparation of SM20:

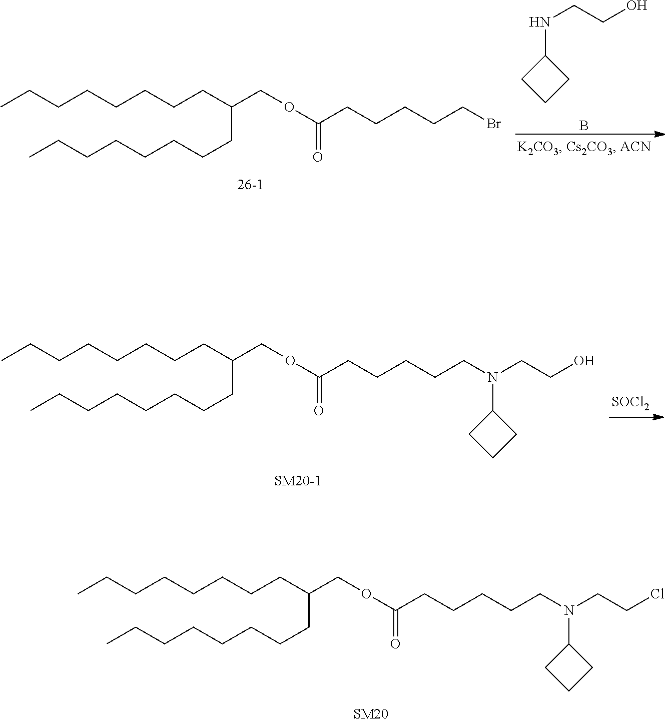

### Step 1: Preparation of Compound SM20-1

To a solution of compound 26-1 (1.0 g, 2.24 mmol, 1.0 eq.) and compound B (511.0 mg, 4.48 mmol, 2.0 eq.) in ACN (20.0 mL) were added Cs₂CO₃ (218.0 mg, 0.67 mmol, 0.3 eq.), K₂CO₃ (927.0 mg, 6.72 mmol, 3.0 eq.) and NaI (33.0 mg, 0.22 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound SM20-1 (0.6 g, 56% yield) as brown oil. LCMS: Rt: 0.950 min; MS m/z (ESI): 482.4 [M+H]⁺.

### Step 2: Preparation of Compound SM20

To a solution of compound SM20-1 (0.2 g, 0.41 mmol, 1.0 eq.) in DCM (5.0 mL) was added SOCl₂ (144.0 mg, 1.23 mmol, 3.0 eq.) at RT. The mixture was stirred for 16 hours. LCMS show the reaction was completed, the mixture was evaporated under reduced pressure to provide compound SM20 (0.23 g, crude) as brown oil. LCMS: Rt: 1.330 min; MS m/z (ESI): 500.3 [M+H]⁺.

### Preparation of SM22:

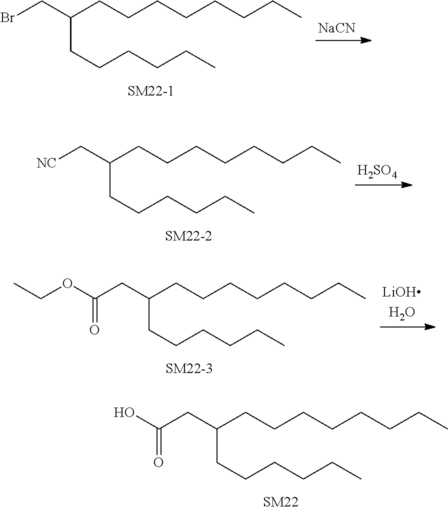

### Step 1: Preparation of Compound SM22-2

To a solution of compound SM22-1 (30.0 g, 98.25 mmol, 1.0 eq.) in DMF (800 mL) was added NaCN (9.63 g, 196.5 mmol, 2.0 eq.). The reaction was stirred at 60° C. for 10 hour. The reaction mixture was poured into water (500 mL) and extracted with EtOAc (3×500 mL). The combined organic layer was washed with brine, dried over anhydrous Na₂SO₄ and concentrated in vacuo. The crude product was purified by flash column chromatography (EtOAc:PE=1:20) to give the target product (18.3 g, 74% yield) as yellow oil.

### Step 2: Preparation of Compound SM22-3

To a solution of compound SM22-2 (17.0 g, 67.61 mmol, 1.0 eq.) in EtOH (200 mL) was added H₂SO₄ (40 mL). The reaction was stirred at 90° C. for 48 hour. The reaction mixture was poured into water (500 mL) and extracted with EtOAc (3×500 mL). The combined organic layer was washed with brine, dried over anhydrous Na₂SO₄ and concentrated in vacuo to give the target product (15 g, 75% yield) as yellow oil.

### Step 3: Preparation of Compound SM22

To a solution of compound SM22-3 (14 g, 46.90 mmol, 1.0 eq.) in MeOH (240 mL) and H₂O (60 mL) was added LiOH·H₂O (9.84 g, 234.5 mmol, 5.0 eq.). The reaction was stirred at 50° C. for 10 hour. The reaction mixture concentrated in vacuo to give the crude target product. The crude product was dissolved in water. The residue was adjusted to pH=2 with 6M HCl and extracted with EtOAc (3×500 mL). The combined organic layer was washed with brine, dried over anhydrous Na₂SO₄ and concentrated in vacuo to give compound SM22 (15 g, 75% yield) as yellow oil. ¹H NMR (400 MHz, CCl₃D): 0.87 (t, J=8 Hz, 6H), 1.22-1.46 (m, 24H), 1.85-1.95 (m, 2H), 2.22-2.34 (m, 1H).

### Preparation of SM23:

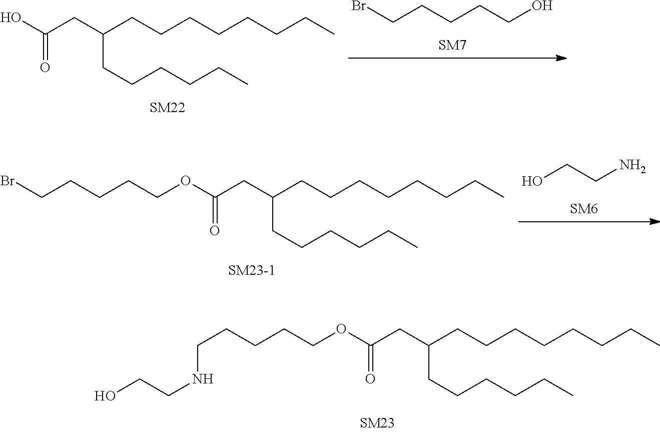

### Step 1: Preparation of Compound SM23-1

To a solution of compound SM22 (4 g, 14.79 mmol, 1.0 eq.) in CH₂Cl₂ (100 mL) was added DIEA (5.73 g, 44.37 mmol, 3.0 eq.), compound SM7 (2.96 g, 17.75 mmol, 1.2 eq.), EDCI (4.25 g, 22.18 mmol, 1.5 eq.), and DMAP (550 mg, 4.44 mmol, 0.3 eq.). The reaction was stirred at 50° C. for 10 hour. The reaction mixture was concentrated in vacuo and purified by flash column chromatography (EtOAc:PE=20:1) to give the target product (4 g, 64% yield) as yellow oil.

### Step 2: Preparation of Compound SM23

To a solution of compound SM23-1 (1.5 g, 3.58 mmol, 1.0 eq.) in CH₃CN (50 mL) was added K₂CO₃ (1.48 g, 10.73 mmol, 3.0 eq.), Cs₂CO₃ (0.4 g, 1.07 mmol, 0.3 eq.), NaI (0.16 g, 1.07 mmol, 0.3 eq.), and compound SM6 (0.45 g, 7.15 mmol, 2.0 eq.). The reaction was stirred at 80° C. for 10 hour. The reaction mixture was concentrated in vacuo. The crude product was purified by flash column chromatography (CH₂Cl₂:MeOH=10:1) to give the target product (800 mg, 56% yield) as yellow oil. LCMS: Rt: 0.898 min; MS m/z (ESI): 400.3 [M+H]⁺.

### Preparation of SM24:

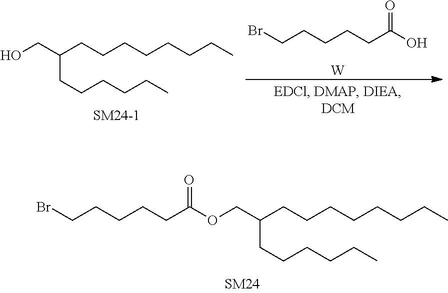

To a solution of compound SM24-1 (20.2 g, 83.3 mmol, 1.0 eq.) and compound W (19.5 g, 100 mol, 1.2 eq.) in DCM (300 mL) was added EDCI (24.0 g, 125 mmol, 1.5 eq.), DMAP (2.0 g, 16.7 mmol, 0.2 eq.) and DIEA (27.0 g, 208 mmol, 2.5 eq.). The reaction mixture was stirred at room temperature for 16 hours. TLC showed the reaction was complete. The reaction mixture was concentrated and purified by column chromatography (silica gel, 0-1% EA in PE) to give compound SM24 (17 g, 49%) as colorless oil.

### Preparation of SM26:

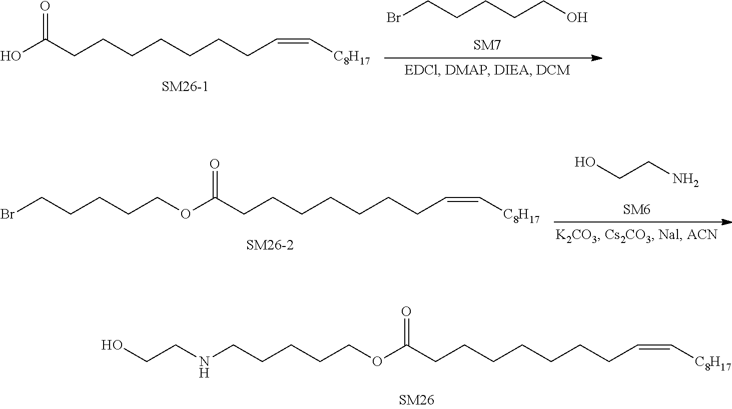

### Step 1: Preparation of Compound SM26-2

To a mixture of compound SM26-1 (2 g, 7.080 mmol, 1.0 eq.) and compound SM7 (1.42 g, 8.496 mmol, 1.2 eq.) were added DIEA (1.8 g, 14.16 mmol, 2.0 eq.), EDCI (2 g, 10.62 mmol, 1.5 eq.), DMAP (0.17 g, 1.416 mmol, 0.2 eq.) in DCM (30 mL). The reaction mixture was stirred at 50° C. for 16 hours. TLC showed the reaction was complete. The mixture power in water and washed with DCM. The organic was separated and dried over Na₂SO₄. Removal of solvent, FCC to get the compound SM26-2 (1.5 g, 49.10%) as yellow oil.

### Step 2: Preparation of Compound SM26

To a solution of compound SM26-2 (1.5 g, 3.476 mmol, 1.0 eq.) in ACN (30 mL) were added compound SM6 (0.64 g, 10.43 mmol, 3.0 eq.), K₂CO₃ (1.4 g, 10.43 mmol, 3.0 eq.), Cs₂CO₃ (0.34 g, 1.043 mmol, 0.3 eq.), NaI (0.16 g, 1.043 mmol, 0.3 eq.). The reaction mixture was stirred at 80° C. for 16 hours. LCMS showed the reaction was complete. Removal of solvent, FCC to get the compound SM26 (800 mg, 55.90%) as yellow oil.

### Preparation of SM30:

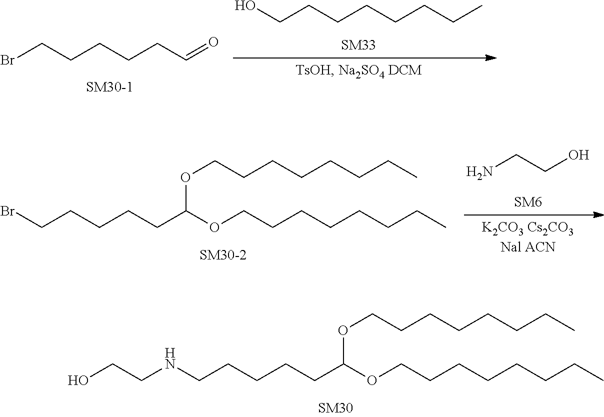

### Step 1: Preparation of Compound SM30-2

To a solution of compound SM30-1 (6.3 g, 35.2 mmol, 1.0 eq.) in DCM (150 mL) were added TsOH·H₂O (1.3 g, 7.0 mmol, 0.2 eq.) and Na₂SO₄ (15.0 g, 105.6 mmol, 3.0 eq.). The mixture was stirred at rt overnight. The mixture was filtered and concentrated. The residue was purified by column chromatography on silica gel (PE/EA=100/1) to give compound SM30-2 (9.7 g, 66% yield) as colorless oil.

### Step 2: Preparation of Compound SM30

To a solution of compound SM30-2 (4.2 g, 10.0 mmol, 1.0 eq.) and ethanolamine (1.8 g, 30.0 mmol, 3.0 eq.) in ACN (50 mL) were added K₂CO₃ (4.1 g, 30.0 mmol, 3.0 eq.), Cs₂CO₃ (977 mg, 3.0 mmol, 0.3 eq.) and NaI (450 mg, 3.0 mmol, 0.3 eq.). The mixture was stirred at 80° C. for 16 hours. LCMS showed the reaction was complete. The mixture was poured into water and extracted with EA. The combined organic layers were washed with brine, dried over Na₂SO₄ and concentrated. The residue was concentrated and purified by column chromatography on silica gel (PE/EA=10/1-3/1-1/1-0/1) to give compound SM30 (2.3 g, 58% yield) as colorless oil. LCMS: Rt: 1.010 min; MS m/z (ESI): 402.4 [M+H]⁺.

### Preparation of SM34:

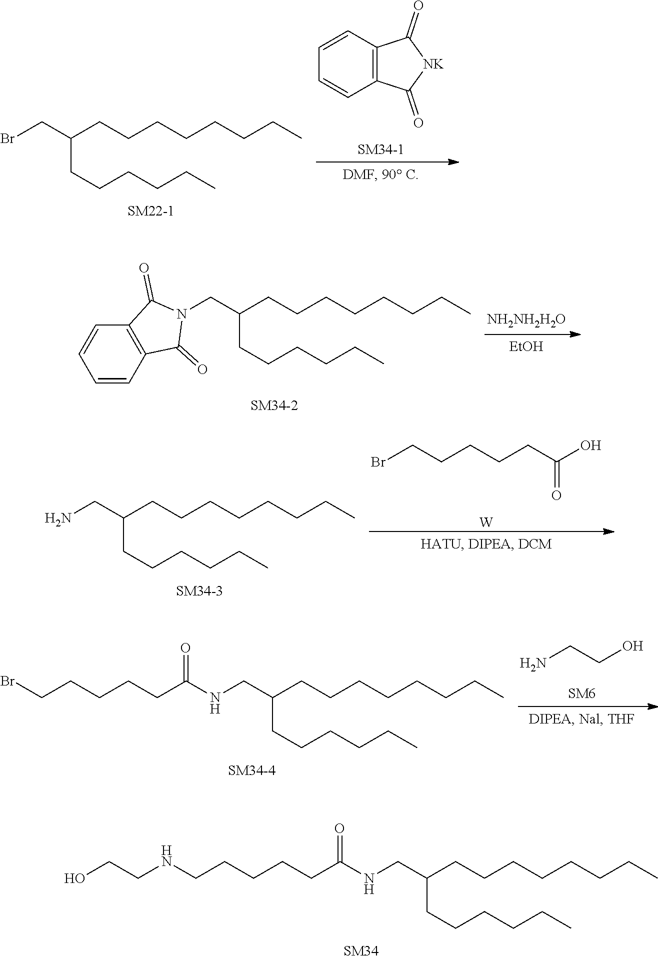

### Step 1: Preparation of Compound SM34-2

To a solution of compound SM22-1 (30 g, 98.2 mmol, 1.0 eq.) in DMF (400 mL) was added compound SM34-1 (36.4 g, 196.4 mmol, 2.0 eq.). The mixture was stirred at 90° C. for 16 hours. The reaction mixture was poured into water and extracted with EA. The combined organic layers were washed with brine, dried over Na₂SO₄ and concentrated. The residue was purified by column chromatography on silica gel (PE/EA=100/1) to give compound SM34-2 (31.6 g, 86% yield) as yellow oil.

### Step 2: Preparation of Compound SM34-3

To a solution of compound SM34-2 (15.8 g, 42.5 mmol, 1.0 eq.) in EtOH (500 mL) was added hydrazine monohydrate (5.0 g, 85.0 mmol, 2.0 eq.). The mixture was stirred at reflux for 16 hours. LCMS showed the reaction was complete. The mixture was filtered and washed with EtOH. The filtrate was concentrated and purified by column chromatography on silica gel (DCM/MeOH=20/1) to give compound SM34-3 (9.1 g, 88% yield) as yellow oil.

### Step 3: Preparation of Compound SM34-4

To a solution of compound SM34-3 (6.5 g, 26.9 mol, 1.2 eq.) in DCM (100 mL) were added compound W (4.4 g, 22.4 mmol, 1.0 eq.), HATU (12.8 g, 33.6 mmol, 1.5 eq.) and DIPEA (8.7 g, 67.2 mmol, 3.0 eq.). The mixture was stirred at room temperature for 16 hours. The reaction mixture was poured into water and extracted with DCM. The combined organic layers were washed with brine, dried over Na₂SO₄ and concentrated. The residue was purified by column chromatography on silica gel to give compound SM34-4 (7.4 g, 65.6% yield) as yellow oil.

### Step 4: Preparation of Compound SM34

To a solution of compound SM34-4 (7.4 g, 18.0 mmol, 1.0 eq.) and compound SM6 (3.3 g, 54.0 mmol, 3.0 eq.) in THF (50 mL) were added DIPEA (6.9 g, 54.0 mmol, 3.0 eq.) and NaI (800 mg, 5.4 mmol, 0.3 eq.). The mixture was stirred at 70° C. for 10 hours. LCMS showed the reaction was complete. The reaction mixture was poured into water and extracted with EA. The combined organic layers were washed with brine, dried over Na₂SO₄ and concentrated. The residue was purified by column chromatography on silica gel to give compound SM34 (6.3 g, 88% yield) as colorless oil. LCMS: Rt: 1.620 min; MS m/z (ESI): 399.5 [M+H]⁺.

### Preparation of SM38:

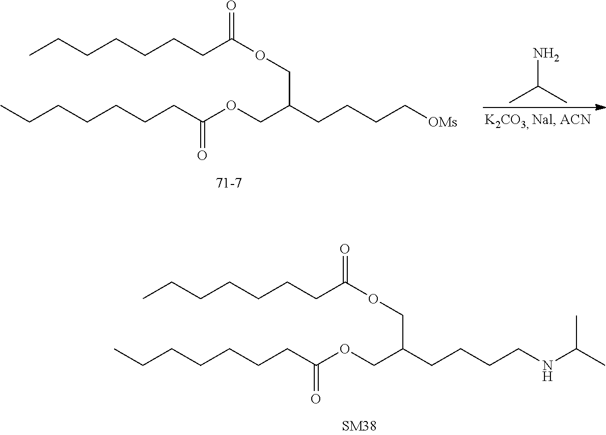

The mixture of compound 71-7 (600 mg, 1.25 mmol, 1.0 eq.), isopropylamine (739 mg, 12.5 mmol, 10.0 eq.), K₂CO₃ (519 mg, 3.76 mmol, 3.0 eq.), Cs₂CO₃ (124 mg, 0.38 mmol, 0.3 eq.), NaI (51 mg, 0.38 mmol, 0.3 eq.) in ACN (10 mL) was stirred at reflux for overnight. LCMS showed the product. The mixture was diluted with EA and washed with water and brine, dried and concentrated. The residue was purified by FCC to give compound SM38 (320 mg, 58.0% yield) as colorless oil.

### Preparation of SM39:

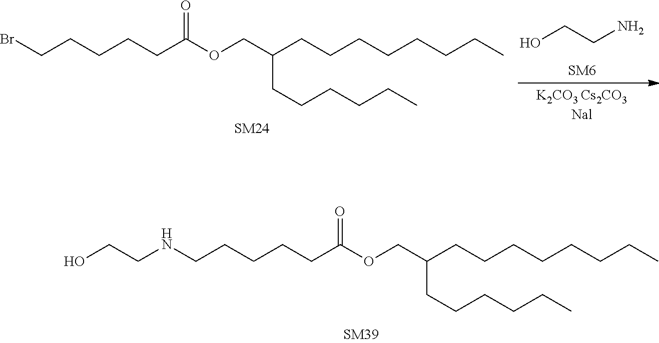

To a solution of compound SM24 (10 g, 23.9 mmol, 1.0 eq.) in CH₃CN (150 mL) were added K₂CO₃ (9.9 g, 71.7 mmol, 3.0 eq.), Cs₂CO₃ (2.3 g, 7.17 mmol, 0.3 eq.), NaI (1.1 g, 7.17 mmol, 0.3 eq.), and compound SM6 (2.9 g, 47.8 mmol, 2.0 eq.). The reaction mixture was stirred at 80° C. for 16 hours. The reaction mixture was concentrated in vacuo. The crude product was purified by flash column chromatography (CH₂Cl₂:MeOH=20:1-10:1) to give compound SM39 as yellow oil (5.1 g, yield: 53%). LCMS: Rt: 0.880 min; MS m/z (ESI): 400.3 [M+H].

### 6.2 Example 2: Preparation of Compound 1

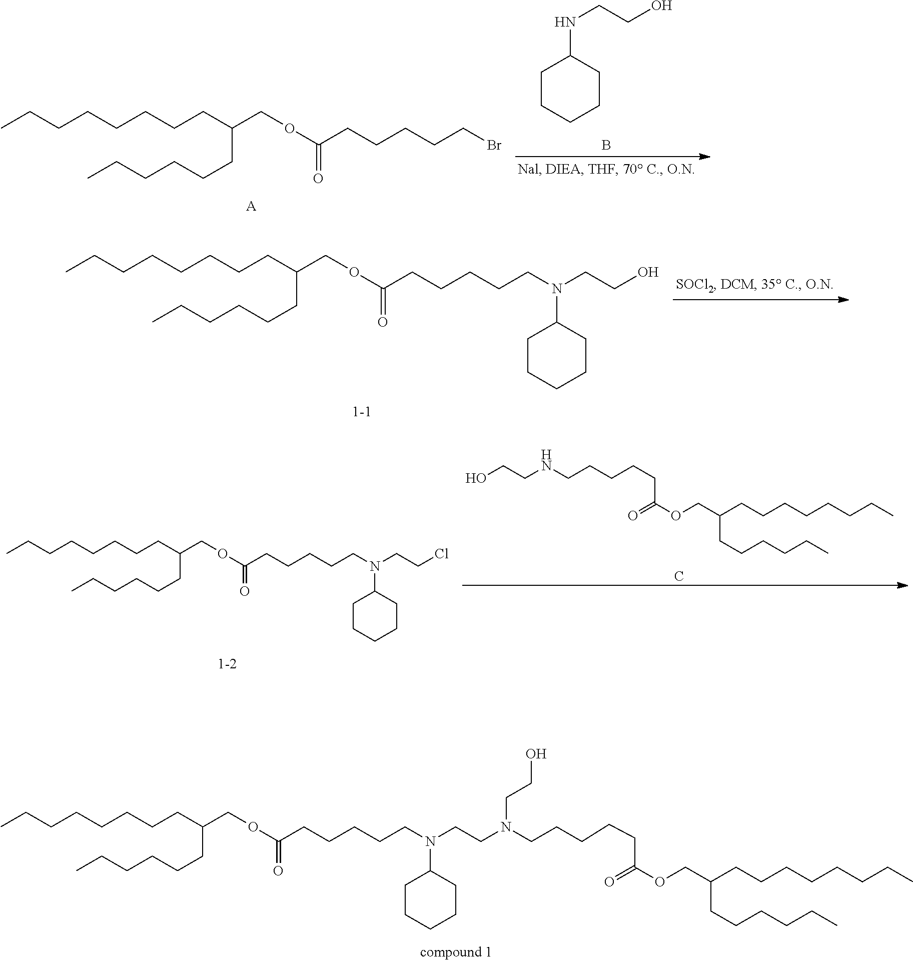

### Step 1: Preparation of Compound 1-1

A mixture of compound A (1.26 g, 3 mmol, 1.5 eq.), compound B (280 mg, 2 mmol, 1 eq.), DIEA (774 mg, 6 mmol, 3 eq.), and NaI (0.1 eq.) in tetrahydrofuran (THF, 6 mL) was stirred overnight at 70° C. The mixture was concentrated under vacuum and purified by silica gel column chromatography (MeOH:DCM=0:1 to 1:80) to provide the desired product compound 1-1 (269 mg, 28.5% yield) as a yellow oil. LCMS: Rt: 1.000 min; MS m/z (ESI): 482.5 [M+H]⁺.

### Step 2: Preparation of Compound 1-2

A mixture of compound 1-1 (269 mg, 0.56 mmol, 1 eq.) and SOCl₂ (200 mg, 1.68 mmol, 3 eq.) in DCM (6 mL) was stirred overnight at 35° C. The mixture was concentrated under vacuum to provide the desired product compound 1-2 (313 mg, crude) as a yellow oil. LCMS: Rt: 0.970 min; MS m/z (ESI): 500.4 [M+H]⁺.

### Step 3: Preparation of Compound 1

A mixture of compound 1-2 (313 mg, 0.63 mmol, 1.2 eq.), compound C (211 mg, 0.53 mmol, 1 eq.), DIEA (205 mg, 1.59 mmol, 3 eq.) and catalyst of NaI in THF (4 mL) was stirred overnight at 70° C. The mixture was concentrated under vacuum, purified by preparatory HPLC to provide compound 1 (79 mg, 14.6% yield) as light brown oil.

¹H NMR (400 MHz, CDCl₃) δ: 0.83-0.93 (m, 12H), 1.04-1.16 (m, 2H), 1.18-1.39 (m, 60H), 1.40-1.55 (m, 3H), 1.56-1.74 (m, 9H), 1.86 (s, 2H), 2.25-2.39 (m, 5H), 2.56 (s, 3H), 2.70 (s, 3H), 3.62 (s, 2H), 3.89-4.04 (m, 4H). LCMS: Rt: 2.000 min; MS m/z (ESI): 863.7 [M+H]⁺.

The following compounds were prepared in analogous fashion as Compound 1, using corresponding starting material.

| Compound | Characterization |
|---|---|
| 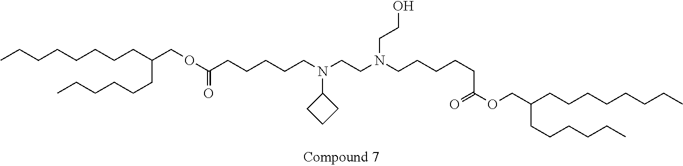 [SMILES: CCCCCCCCC(CCCCCC)COC(=O)CCCCCN(CCO)CCN(CCCCCC(=O)OCC(CCCCCC)CCCCCCCC)C1CCC1] | ¹H NMR (400 MHz, CDCl₃) δ: 3.97 (d, J = 5.6 Hz, 4H), 3.55- 3.53 (m, 2H), 3.09-3.05 (m, 1H), 2.61-2.58 (m, 2H), 2.55-2.50 (m, 5H), 2.48-2.40 (m, 4H), 2.32- 2.28 (m, 4H), 2.02-1.88 (m, 5H), 1.7-1.54 (m, 10H), 1.50-1.38 (m, 6H), 1.27 (s, 47H), 0.90-0.87 (m, 12H). LCMS: Rt: 1.340 min; MS m/z (ESI): 835.7 [M + H]⁺. |

### 6.3 Example 3: Preparation of Compound 2

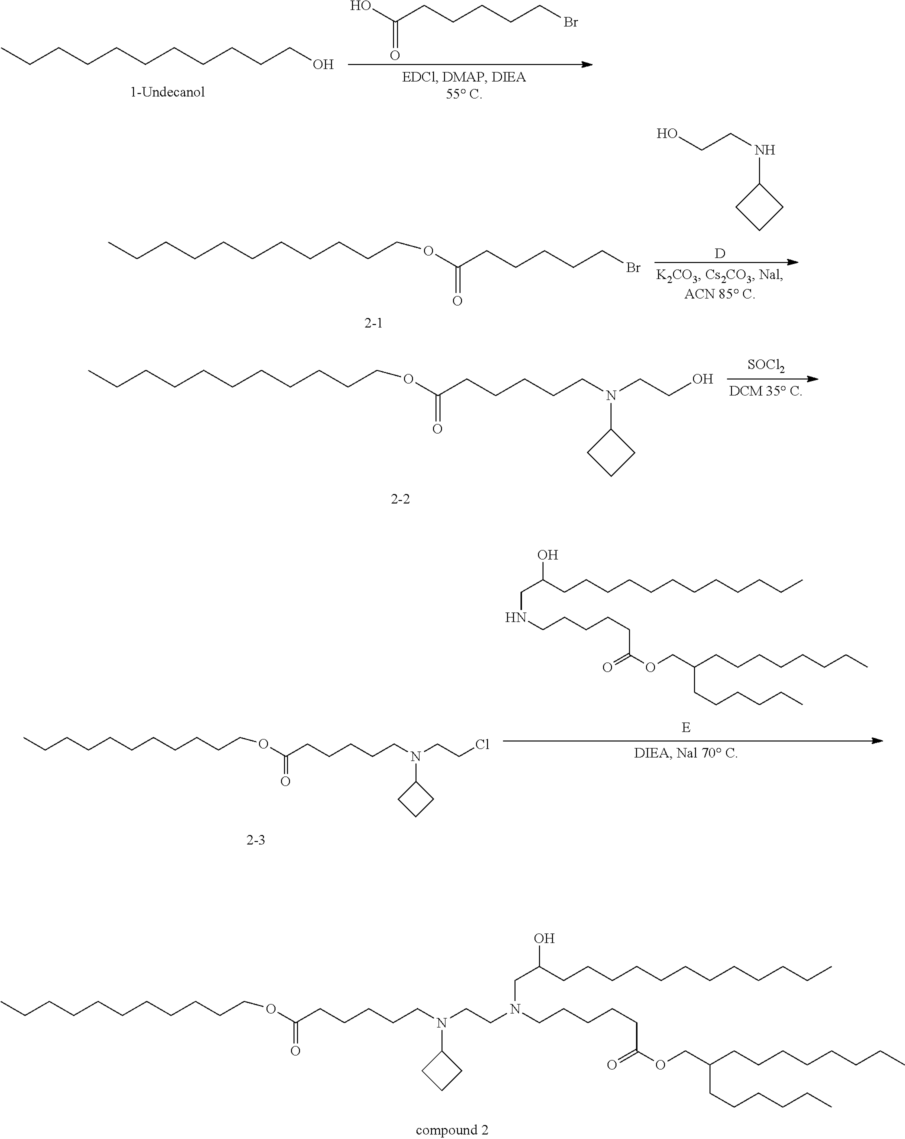

### Step 1: Preparation of Compound 2-1

To a solution of 1-undecanol (10 g, 58.03 mmol, 1.0 eq.) in DCM (120 mL) was added 1-ethyl-3-(3-dimethylaminopropyl)carbodiimide (EDCI, 16.69 g, 87.05 mmol, 1.5 eq.), 4-dimethylaminopyridine (DMAP, 1.42 g, 11.61 mmol, 0.2 eq.), DIEA (15 g, 116.06 mmol, 2.0 eq.), and 6-bromohexanoic acid (12.45 g, 63.84 mmol, 1.1 eq.). The reaction mixture was stirred at 55° C. for 16 hours. TLC showed the reaction was complete. Removal of solvent and purification of the crude product by FCC provided compound 2-1 (8.6 g, 42.43%) as colorless oil.

¹H NMR (400 MHz, CDCl₃) δ: 4.08-4.05 (m, 2H), 3.55-3.52 (m, 2H), 2.34-2.30 (m, 2H), 1.83-1.76 (m, 2H), 1.69-1.60 (m, 4H), 1.51-1.43 (m, 2H), 1.23 (s, 16H), 0.89-0.86 (m, 3H).

### Step 2: Preparation of Compound 2-2

To a solution of compound 2-1 (1 g, 2.863 mmol, 1.2 eq.) in ACN (20 mL) was added compound D (275 mg, 2.386 mmol, 1.0 eq.), K₂CO₃ (989 mg, 7.158 mmol, 3.0 eq.), Cs₂CO₃ (233 mg, 0.7158 mmol, 0.3 eq.), and NaI (18 mg, 0.1193 mmol, 0.05 eq.). The reaction mixture was stirred at 85° C. for 16 hours. LCMS showed the reaction was complete. Removal of solvent and purification of the crude product by FCC provided compound 2-2 (170 mg, 18.57%) as a yellow oil. LCMS: Rt: 0.811 min; MS m/z (ESI): 384.3 [M+H]⁺.

### Step 3: Preparation of Compound 2-3

To a solution of compound 2-2 (170 mg, 0.4432 mmol, 1.0 eq.) in DCM (10 mL) was added SOCl₂ (158 mg, 1.330 mmol, 3.0 eq.). The reaction mixture was stirred at 35° C. for 16 hours. LCMS showed the reaction was complete. Removal of solvent provided compound 2-3 (180 mg, crude) as a yellow oil. LCMS: Rt: 0.860 min; MS m/z (ESI): 402.3 [M+H]⁺.

### Step 4: Preparation of Compound 2

To a mixture of compound 2-3 (170 mg, 0.4476 mmol, 1.0 eq.) and DIEA (289 mg, 2.238 mmol, 5.0 eq.) in THF (10 mL) was added compound E (381 mg, 0.6715 mmol, 1.5 eq.) and NaI (20 mg). The reaction mixture was stirred at 70° C. for 16 hours. LCMS showed the reaction was complete. After removal of solvent, purification by prep-HPLC provided compound 2 (35 mg, 8.37% yield) as colorless oil.

¹H NMR (400 MHz, CDCl₃) δ: 4.07-4.04 (m, 2H), 3.9 (d, J=5.6 Hz, 2H), 3.53 (m, 1H), 3.08-3.04 (m, 1H), 2.49-2.37 (m, 9H), 2.32-2.25 (m, 5H), 1.98-1.88 (m, 4H), 1.66-1.58 (m, 9H), 1.49-1.38 (m, 7H), 1.26 (s, 63H), 0.90-0.86 (m, 12H). LCMS: Rt: 0.994 min; MS m/z (ESI): 933.8 [M+H]⁺.

### 6.4 Example 4: Preparation of Compound 3

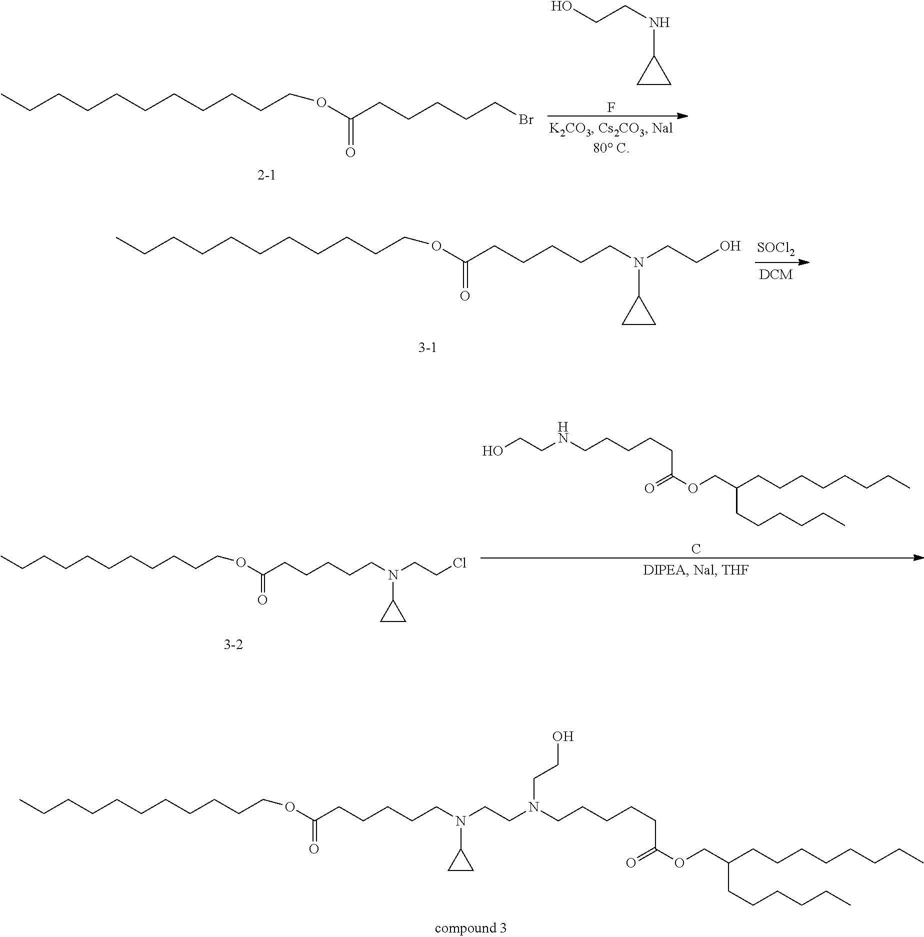

### Step 1: Preparation of Compound 3-1

To a solution of compound 2-1 (1.0 g, 2.86 mmol, 2.0 eq.) and compound F (145 mg, 1.43 mmol, 1.0 eq.) in ACN (30 mL) were added K₂CO₃ (593 mg, 4.29 mmol, 3.0 eq.), Cs₂CO₃ (140 mg, 0.429 mmol, 0.3 eq.) and NaI (64 mg, 0.429 mmol, 0.3 eq.). The mixture was stirred at 80° C. for 48 hours. LCMS showed the reaction was complete. The reaction mixture was concentrated and purified by silica gel column chromatography (DCM/MeOH=50/1-25/1) to provide compound 3-1 (350 mg, 66% yield) as a yellow oil. LCMS: Rt: 0.800 min; MS m/z (ESI): 370.3 [M+H]⁺.

### Step 2: Preparation of Compound 3-2

To a solution of compound 3-1 (200 mg, 0.54 mmol, 1.0 eq.) in DCM (10 mL) was added SOCl₂ (193 mg, 1.62 mmol, 3.0 eq.). The mixture was stirred at 30° C. for 16 hours. LCMS show the reaction was complete. The mixture was concentrated under reduced pressure to provide compound 3-2 (200 mg, 95%) as a yellow oil.

### Step 3: Preparation of Compound 3

To a solution of compound 3-2 (200 mg, 0.52 mmol, 1.0 eq.) and compound C (416 mg, 1.04 mmol, 2.0 eq.) in THF (10 mL) were added N,N-diisopropylethylamine (DIPEA, (202 mg, 1.56 mmol, 3.0 eq.) and NaI (24 mg, 0.16 mmol, 0.3 eq.). The mixture was stirred at 70° C. for 16 hours. LCMS show the reaction was completed. The mixture was concentrated and purified by prep-HPLC to provide compound 3 (80 mg, 8% yield) as a yellow oil.

¹H NMR (400 MHz, CDCl₃) δ: 0.48-0.50 (m, 4H), 0.86-0.90 (m, 9H), 1.26-1.30 (m, 45H), 1.49-1.66 (m, 11H), 1.72-1.77 (m, 1H), 2.28-2.32 (m, 4H), 2.52-2.76 (m, 10H), 3.52-3.58 (m, 2H), 3.96-3.98 (m, 2H), 4.04-4.07 (m, 2H). LCMS: Rt: 1.250 min; MS m/z (ESI): 751.6 [M+H]⁺.

The following compounds were prepared in analogous fashion as Compound 3, using corresponding starting material.

| Compound | Characterization |
|---|---|
| 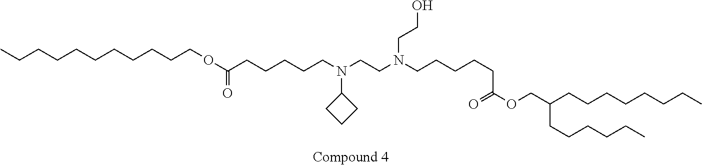 [SMILES: CCCCCCCCCCCOC(=O)CCCCCN(CCN(CCO)CCCCCC(=O)OCC(CCCCCC)CCCCCCCC)C1CCC1] | ¹H NMR (400 MHz, CDCl₃) δ: 0.83-0.93 (m, 9H), 1.15-1.33 (m, 42H), 1.35-1.45 (m, 3H), 1.50- 1.87 (m, 23H), 1.92-2.04 (m, 2H), 2.26-2.29 (m, 2H), 2.35- 2.39 (m, 3H), 2.60-2.79 (m, 1H), 2.92-3.04 (m, 2H), 3.48-3.65 (m, 1H), 3.91-3.99 (m, 2H), 4.02- 4.10 (m, 2H). LCMS: Rt: 1.620 min; MS m/z (ESI): 765.6 [M + H]⁺. |
| 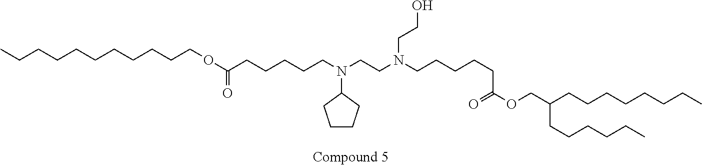 [SMILES: CCCCCCCCCCCOC(=O)CCCCCN(CCN(CCO)CCCCCC(=O)OCC(CCCCCC)CCCCCCCC)C1CCCC1] | ¹H NMR (400 MHz, CDCl₃) δ: 0.86-0.90 (m, 9H), 1.26-1.35 (m, 47H), 1.45-1.69 (m, 16H), 1.77- 1.89 (m, 2H), 2.29-2.32 (m, 4H), 2.49-2.78 (m, 10H), 3.56-3.61 (m, 2H), 3.95-3.97 (m, 2H), 4.04-4.07 (m, 2H). LCMS: Rt: 1.140 min; MS m/z (ESI): 779.7 [M + H]⁺. |
| 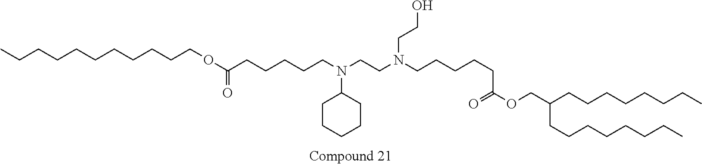 [SMILES: CCCCCCCCCCCOC(=O)CCCCCN(CCN(CCO)CCCCCC(=O)OCC(CCCCCCCC)CCCCCCCC)C1CCCCC1] | ¹H NMR (400 MHz, CDCl₃) δ: 0.86-0.89 (m, 9H), 1.21-1.35 (m, 54H), 1.47-1.51 (m, 4H), 1.58- 1.66 (m, 8H), 1.67-1.80 (m, 4H), 2.28-2.33 (m, 4H), 2.48-2.61 (m, 11H), 3.53-3.55 (m, 2H), 3.96- 4.08 (m, 4H). LCMS: Rt: 1.520 min; MS m/z (ESI): 821.7 [M + H]⁺. |

### 6.5 Example 5: Preparation of Compound 6

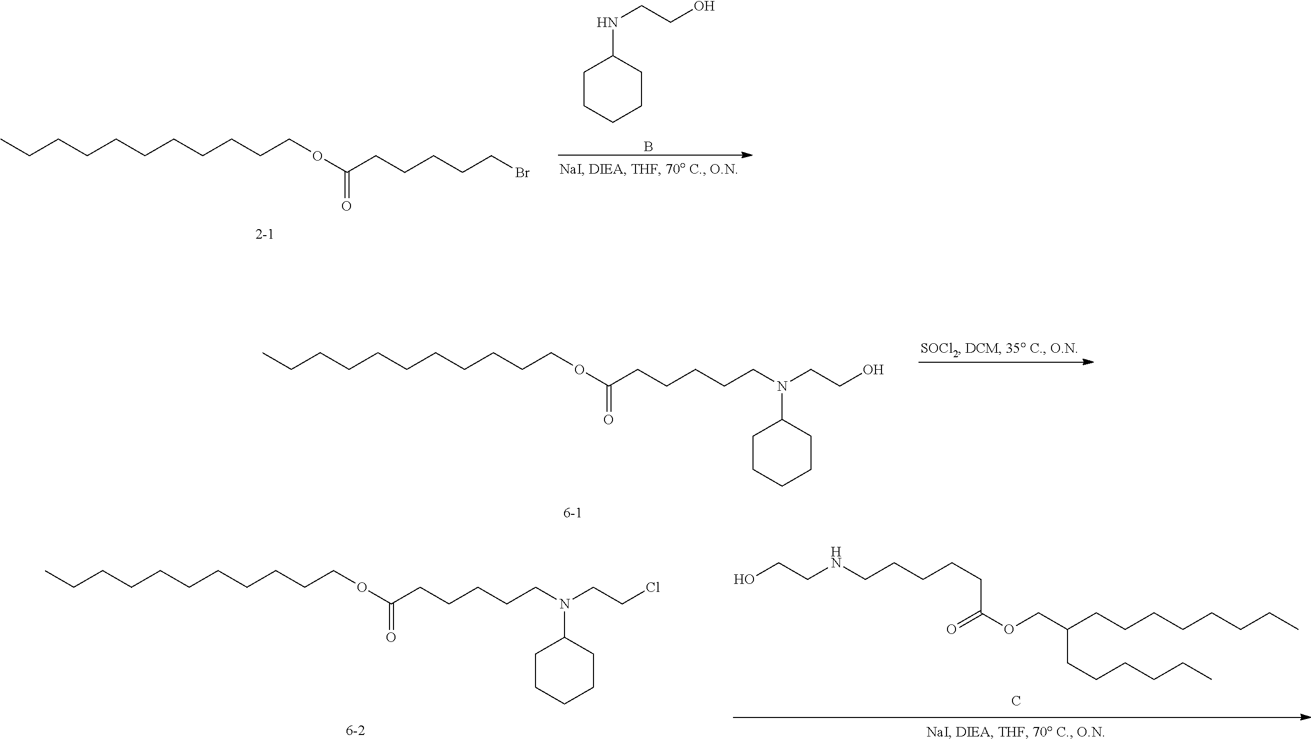

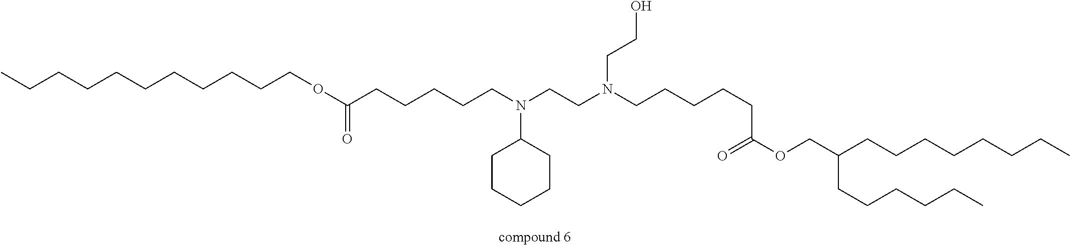 [SMILES: CCCCCCCCCCCOC(=O)CCCCCN(CCN(CCO)CCCCCC(=O)OCC(CCCCCC)CCCCCCCC)C1CCCCC1]

### Step 1: Preparation of Compound 6-1

A mixture of compound 2-1 (786 mg, 2.24 mmol, 1.2 eq.), compound B (268 mg, 1.87 mol, 1 eq.), DIEA (724 mg, 5.61 mmol, 3 eq.), and NaI (0.1 eq.) in THF (10 mL) was stirred overnight at 70° C. The mixture was concentrated under vacuum and purified by silica gel column chromatography (MeOH:DCM=0:1 to 1:20) to provide compound 6-1 (1.18 g, crude) as light brown oil. LCMS: Rt: 0.910 min; MS m/z (ESI): 412.3 [M+H]⁺.

### Step 2: Preparation of Compound 6-2

A mixture of compound 6-1 (412 mg, 1 mmol, 1 eq.) and SOCl₂ (357 mg, 3 mmol, 3 eq.) in DCM (6 mL) was stirred overnight at 35° C. The mixture was concentrated under vacuum to provide compound 6-2 (430 mg, crude) as a yellow oil. LCMS: Rt: 0.930 min; MS m/z (ESI): 430.3 [M+H]⁺.

### Step 3: Preparation of Compound 6

A mixture of compound 6-2 (215 mg, 0.5 mmol, 1 eq.), compound C (150 mg, 0.4 mmol, 0.75 eq.), DIEA (195 mg, 1.5 mmol, 3 eq.) and catalytic amount of NaI in THF (3 mL) was stirred overnight at 70° C. The mixture was concentrated under vacuum, purified by prep-HPLC to provide compound 6 (15 mg, 12.8% yield) as light brown oil.

¹H NMR (400 MHz, CDCl₃) δ: 0.83-0.92 (m, 9H), 1.18-1.36 (m, 40H), 1.38-1.48 (m, 4H), 1.49-1.75 (m, 27H), 1.85-2.15 (m, 5H), 2.16-2.27 (m, 1H), 2.30-2.39 (m, 3H), 3.11-3.25 (m, 2H), 3.35-3.48 (m, 1H), 3.93-3.99 (m, 2H), 4.01-4.11 (m, 2H). LCMS: Rt: 1.720 min; MS m/z (ESI): 793.6 [M+H]⁺.

### 6.6 Example 6: Preparation of Compound 8

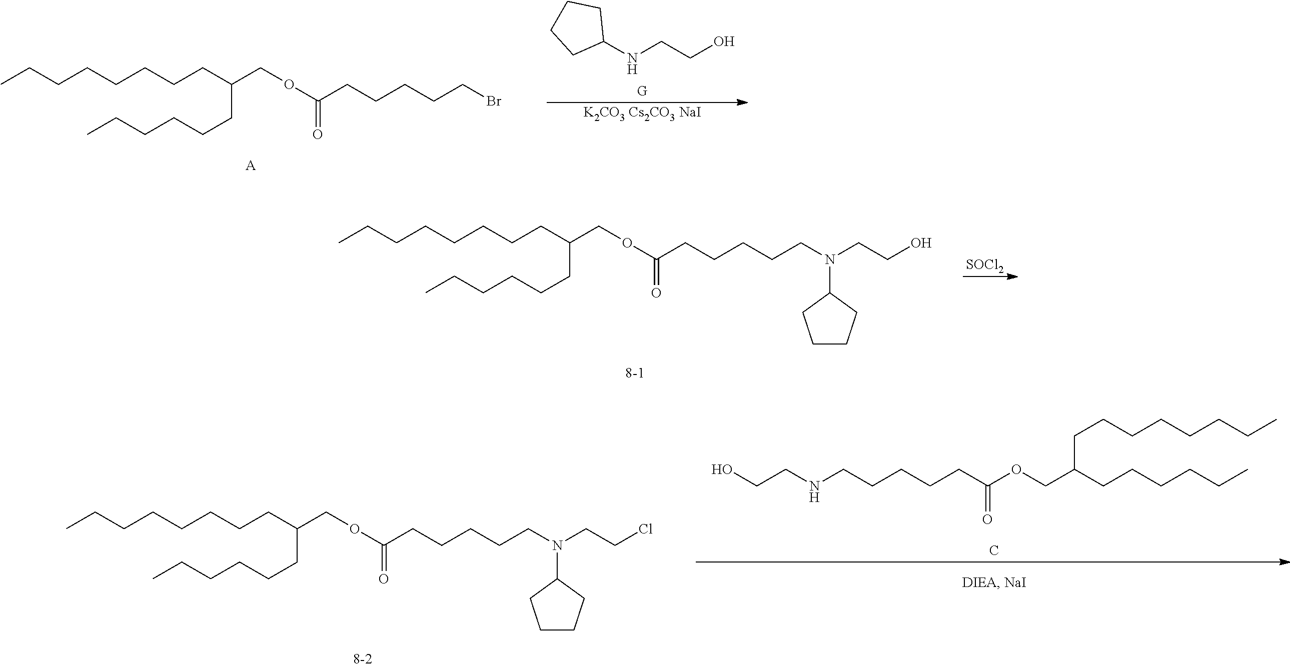

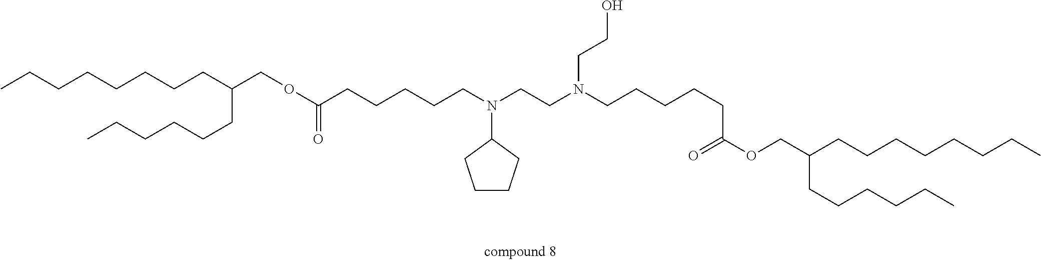 [SMILES: CCCCCCCCC(CCCCCC)COC(=O)CCCCCN(CCO)CCN(CCCCCC(=O)OCC(CCCCCC)CCCCCCCC)C1CCCC1]

### Step 1: Preparation of 8-1

To a solution of compound A (0.85 g, 1.98 mmol) in CH₃CN (50 mL) was added K₂CO₃ (410 mg, 2.97 mmol), Cs₂CO₃ (100 mg, 0.29 mmol), NaI (50 mg, 0.29 mmol) and compound G (127 mg, 0.99 mmol). The reaction was stirred at 80° C. for 10 hour. The reaction mixture was concentrated in vacuo. The crude product was purified by flash column chromatography (CH₂Cl₂:MeOH=10:1) to provide compound 8-1 as a yellow oil (300 mg, yield: 65%). LCMS: Rt: 0.88 min; MS m/z (ESI): 468.4 [M+H]⁺.

### Step 2: Preparation of Compound 8-2

To a solution of compound 8-1 (300 mg, 0.64 mmol) in CH₂Cl₂ (10 mL) was added SOCl₂ (250 mg, 2.05 mmol). The reaction was stirred at 30° C. for 10 hours. The reaction mixture concentrated in vacuo to provide compound 8-2 as a yellow oil (310 mg, yield: 100%).

### Step 3: Preparation of Compound 8

To a solution of compound 8-2 (300 mg, 0.62 mmol) in THF (10 mL) were added DIEA (240 mg, 1.85 mmol), NaI (100 mg, 0.65 mmol) and compound C (530 mg, 1.31 mmol). The reaction was stirred at 70° C. for 10 hour. The reaction mixture was filtrated and concentrated in vacuo. The crude product was purified by prep-HPLC to provide compound 8 as a yellow oil (45 mg, yield: 8.5%).

¹H NMR (400 MHz, CDCl₃) δ: 0.87-0.90 (t, J=6.8 Hz, 12H), 1.26 (m, 50H), 1.40-1.51 (m, 8H), 1.60-1.66 (m, 8H), 1.77-1.73 (m, 3H), 2.31-2.33 (m, 4H), 2.48-2.61 (m, 10H), 3.05-3.09 (m, 1H), 3.48-3.55 (m, 4H), 3.96-3.97 (m, 4H). LCMS: Rt: 1.740 min; MS m/z (ESI): 849.7 [M+H]⁺.

The following compounds were prepared in analogous fashion as Compound 8, using corresponding starting material.

| Compound | Characterization |
|---|---|
| 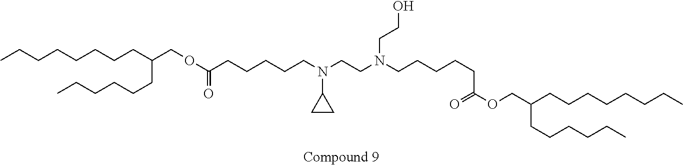 [SMILES: CCCCCCCCC(CCCCCC)COC(=O)CCCCCN(CCO)CCN(CCCCCC(=O)OCC(CCCCCC)CCCCCCCC)C1CC1] | ¹H NMR (400 MHz, CDCl₃) δ: 0.43-0.47 (m, 4H), 0.87 (t, J = 8 Hz, 12H), 1.11-1.32 (m, 50H), 1.45-1.72 (m, 13H), 2.29-2.33 (m, 4H), 2.48-2.66 (m, 11H), 3.48-3.51 (m, 2H), 3.96-3.98 (m, 4H). LCMS: Rt: 1.384 min; MS m/z (ESI): 821.7 [M + H]⁺. |
| 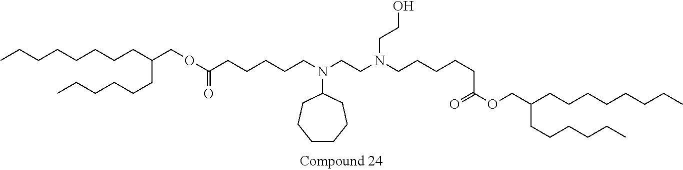 [SMILES: CCCCCCCCC(CCCCCC)COC(=O)CCCCCN(CCO)CCN(CCCCCC(=O)OCC(CCCCCC)CCCCCCCC)C1CCCCCC1] | ¹H NMR (400 MHz, CDCl₃) δ: 3.97 (d, J = 5.6 Hz, 4H), 3.54 (s, 2H), 2.60-2.48 (m, 9H), 2.33- 2.29 (m, 4H), 1.82 (s, 2H), 1.68- 1.60 (m, 8H), 1.53-1.40 (m, 12H), 1.26 (s, 56H), 0.90-0.87 (m, 12H). LCMS: Rt: 1.770 min; MS m/z (ESI): 877.7 [M + H]⁺. |

### 6.7 Example 7: Preparation of Compound 10

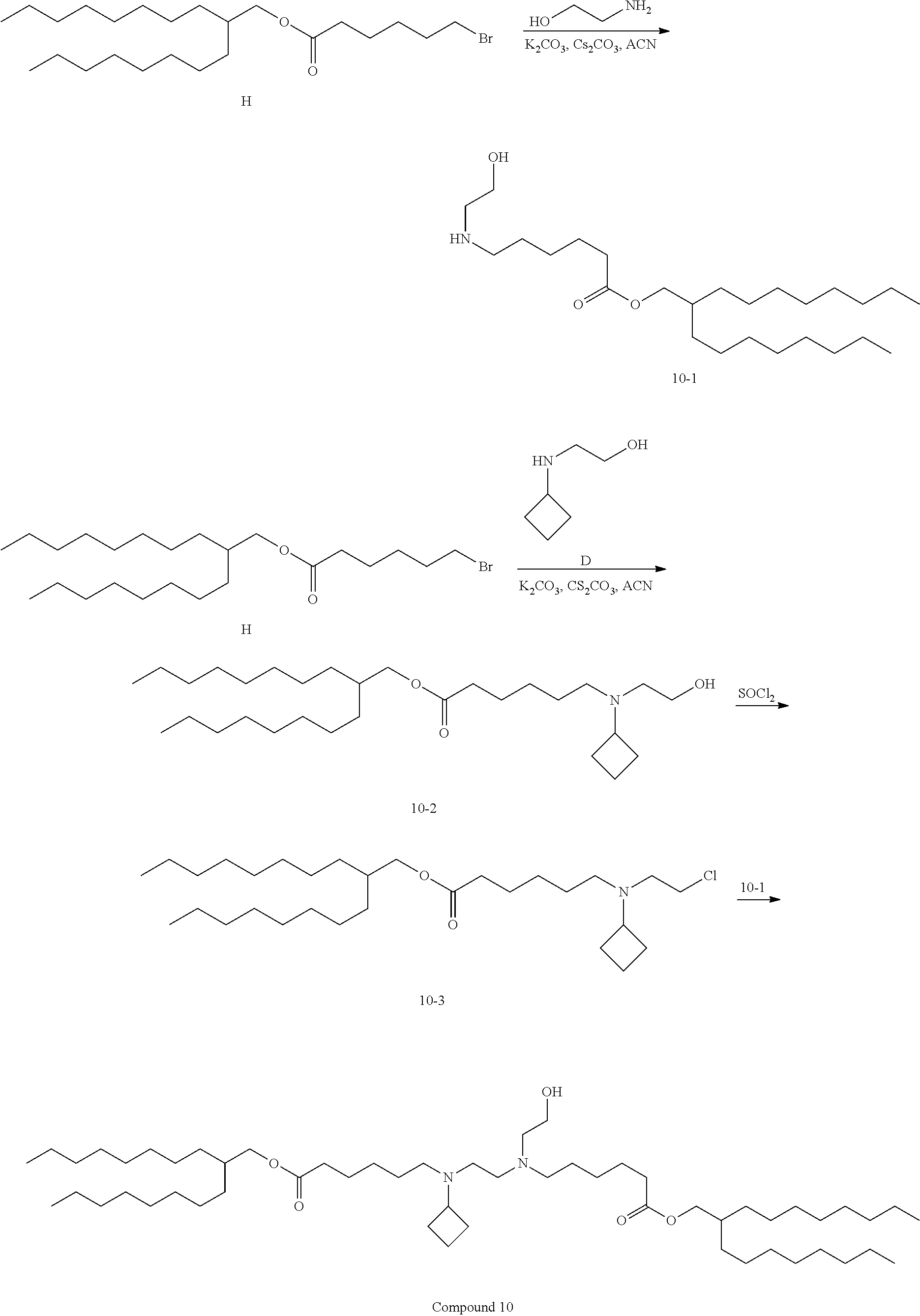

### Step 1: Preparation of Compound 10-1

To a solution of compound H (446.0 mg, 1.0 mmol, 1.0 eq.) and ethanolamine (180.0 mg, 3.0 mmol, 3.0 eq.) in ACN (10.0 mL) were added Cs₂CO₃ (97.5 mg, 0.3 mmol, 0.3 eq.), K₂CO₃ (414.0 mg, 3.0 mmol, 3.0 eq.) and NaI (14.6 mg, 0.1 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound 10-1 (0.35 g, 82% yield) as a yellow oil. LCMS: Rt: 0.942 min; MS m/z (ESI): 428.3 [M+H]⁺.

### Step 2: Preparation of Compound 10-2

To a solution of compound H (1.0 g, 2.24 mmol, 1.0 eq.) and compound D (511.0 mg, 4.48 mmol, 2.0 eq.) in ACN (20.0 mL) were added Cs₂CO₃ (218.0 mg, 0.67 mmol, 0.3 eq.), K₂CO₃ (927.0 mg, 6.72 mmol, 3.0 eq.) and NaI (33.0 mg, 0.22 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound 10-2 (0.6 g, 56% yield) as brown oil. LCMS: Rt: 0.950 min; MS m/z (ESI): 482.4 [M+H]⁺.

### Step 3: Preparation of Compound 10-3

To a solution of compound 10-2 (0.2 g, 0.41 mmol, 1.0 eq.) in DCM (5.0 mL) was added SOCl₂ (144.0 mg, 1.23 mmol, 3.0 eq.) at RT. The mixture was stirred for 16 hours. LCMS show the reaction was completed, the mixture was evaporated under reduced pressure to provide compound 10-3 (0.23 g, crude) as brown oil. LCMS: Rt: 1.330 min; MS m/z (ESI): 500.3 [M+H]⁺.

### Step 4: Preparation of Compound 10

To a solution of compound 10-3 (150.0 mg, 0.3 mmol, 1.0 eq.) and compound 10-1 (192.0 mg, 0.45 mmol, 1.5 eq.) in THF (5.0 mL) was added DIEA (193 mg, 1.5 mmol, 5.0 eq.) at 0° C. The mixture was stirred for 16 hours at 70° C. LCMS show the reaction was completed, the mixture was evaporated under reduced pressured purified with pre-HPLC to provide compound 10 (80.0 mg, 25% yield) as brown oil.

¹H NMR (400 MHz, CDCl₃) δ: 0.86-0.89 (m, 12H), 1.26-1.32 (m, 61H), 1.41-1.65 (m, 12H), 1.85-2.02 (m, 4H), 2.28-2.61 (m, 14H), 3.00-3.12 (m, 1H), 3.53-3.55 (m, 2H), 3.97 (d, J=5.6 Hz, 4H). LCMS: Rt: 2.520 min; MS m/z (ESI): 891.7 [M+H]⁺.

### 6.8 Example 8: Preparation of Compound 11

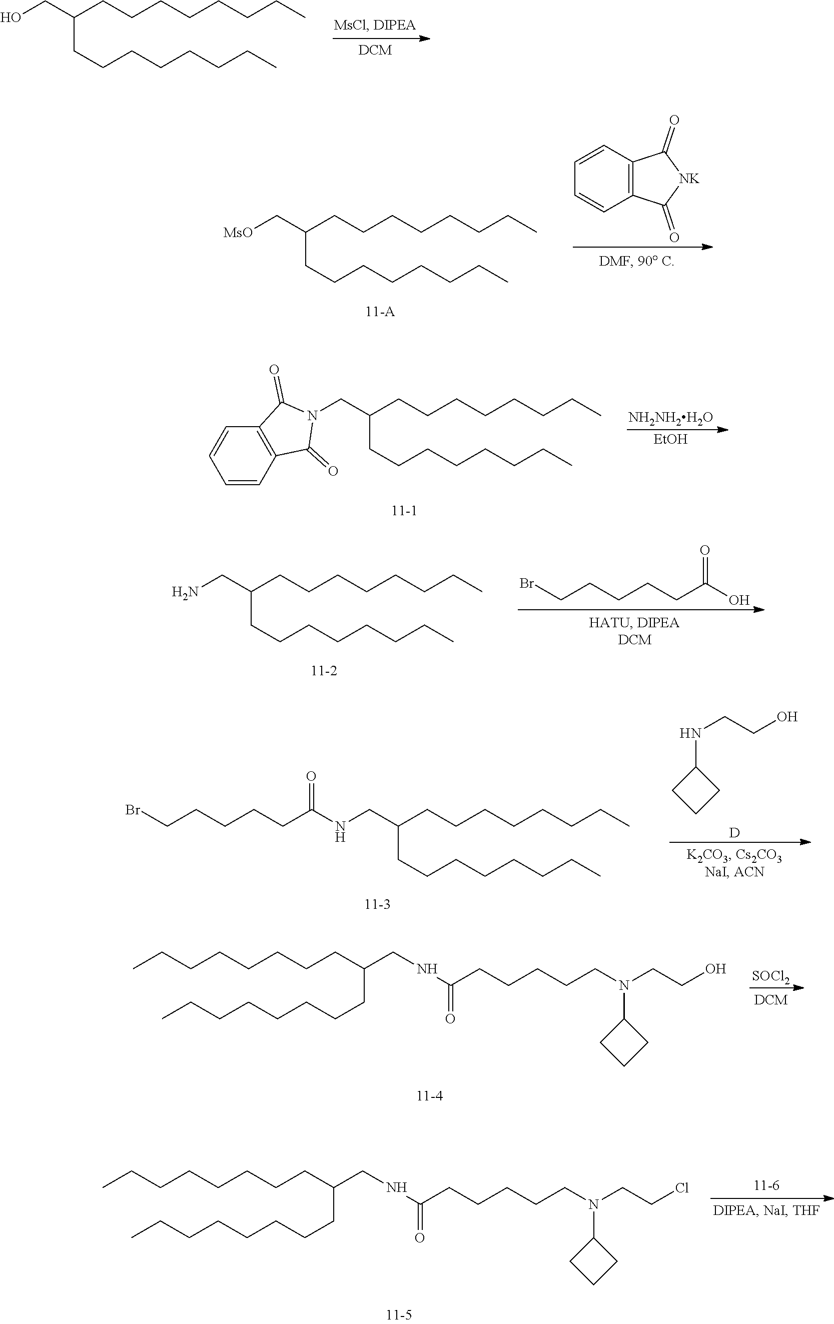

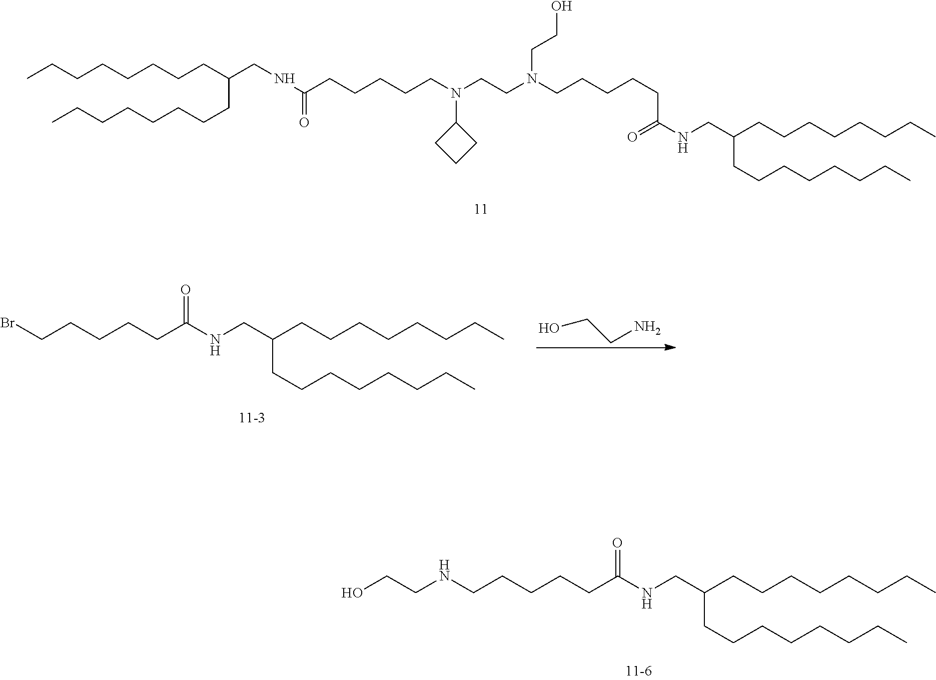

### Step 1: Preparation of Compound 11-A

To a solution of 2-octyl-1-decanol (2.7 g, 10.0 mmol, 1.0 eq.) and DIPEA (2.6 g, 20.0 mmol, 2.0 eq.) in DCM (50 mL) at 0° C. was added methanesulfonyl chloride (MsCl, 1.4 g, 12.0 mmol, 1.2 eq.) dropwise. The mixture was stirred at room temperature for 2 hours. The reaction mixture was washed with water, brine, dried over Na₂SO₄ and concentrated to provide compound 11-A (3.1 g, 91% yield) as a yellow oil. ¹H NMR (400 MHz, CDCl₃) δ: 0.86-0.90 (m, 6H), 1.26-1.32 (m, 29H), 3.00 (s, 3H), 4.11-4.13 (m, 2H).

### Step 2: Preparation of Compound 11-1

To a solution of 11-A (18.0 g, 51.6 mmol, 1.0 eq.) in DMF (300 mL) was added potassium phthalimide (19.1 g, 103.2 mmol, 2.0 eq.). The mixture was stirred at 90° C. for 16 hours. The reaction mixture was poured into water and extracted with EA. The combined organic layers were washed with brine, dried over Na₂SO₄ and concentrated. Purification by silica gel column chromatography (PE/EA=100/1) provided compound 11-1 (14.6 g, 71% yield) as colorless oil. ¹H NMR (400 MHz, CDCl₃) δ: 0.85-0.88 (m, 6H), 1.24-1.29 (m, 28H), 1.82-1.89 (m, 1H), 3.56-3.58 (m, 2H), 7.72-7.72 (m, 2H), 7.83-7.85 (m, 2H).

### Step 3: Preparation of Compound 11-2

To a solution of compound 11-1 (14.6 g, 36.5 mmol, 1.0 eq.) in EtOH (400 mL) was added hydrazine monohydrate (3.65 g, 73.0 mmol, 2.0 eq.). The mixture was stirred at reflux for 16 hours. LCMS showed the reaction was complete. The mixture was filtered and washed with EtOH. The filtrate was concentrated and purified by silica gel column chromatography (DCM/MeOH=100/1-50/1) to provide compound 11-2 (6.9 g, 70% yield) as a yellow oil. LCMS: Rt: 1.260 min; MS m/z (ESI): 270.3 [M+H]⁺.

### Step 4: Preparation of Compound 11-3

To a solution of compound 11-2 (6.9 g, 25.6 mmol, 1.0 eq.) in DCM (250 mL) were added 6-bromohexanoic acid (6.0 g, 30.7 mmol, 1.2 eq.), HATU (11.7 g, 30.7 mmol, 1.2 eq.) and DIPEA (9.9 g, 76.8 mmol, 3.0 eq.). The mixture was stirred at RT for 16 hours. The reaction mixture was poured into water and extracted with DCM. The combined organic layers were washed with brine, dried over Na₂SO₄ and concentrated. Purification by silica gel column chromatography (PE/EA=10/1-8/1) provided compound 11-3 (7.1 g, 62% yield) as a yellow oil.

### Step 5: Preparation of Compound 11-4

To a solution of compound 11-3 (800 mg, 1.79 mmol, 1.5 eq.) and compound D (137 mg, 1.19 mmol, 1.0 eq.) in ACN (40 mL) were added K₂CO₃ (493 mg, 3.57 mmol, 3.0 eq.), Cs₂CO₃ (116 mg, 0.357 mmol, 0.3 eq.) and NaI (54 mg, 0.357 mmol, 0.3 eq.). The mixture was stirred at 80° C. for 16 hours. LCMS showed the reaction was complete. The reaction mixture was concentrated and purified by silica gel column chromatography (DCM/MeOH=10/1) to provide compound 11-4 (400 mg, 70% yield) as a yellow oil. LCMS: Rt: 0.920 min; MS m/z (ESI): 481.4 [M+H]⁺.

### Step 6: Preparation of Compound 11-5

To a solution of compound 11-4 (200 mg, 0.42 mmol, 1.0 eq.) in DCM (10 mL) was added SOCl₂ (150 mg, 1.26 mmol, 3.0 eq.). The mixture was stirred at 30° C. for 16 hours. LCMS showed the reaction was complete. The mixture was concentrated under reduced pressure to provide compound 11-5 (200 mg, 95%) as a yellow oil. LCMS: Rt: 0.980 min; MS m/z (ESI): 499.3 [M+H]⁺.

### Step 7: Preparation of Compound 11-6

To a solution of compound 11-3 (610 mg, 1.36 mmol, 1.0 eq.) and ethanolamine (166 mg, 2.72 mmol, 2.0 eq.) in ACN (20 mL) were added K₂CO₃ (564 mg, 4.08 mmol, 3.0 eq.), Cs₂CO₃ (134 mg, 0.41 mmol, 0.3 eq.) and NaI (61 mg, 0.41 mmol, 0.3 eq.). The mixture was stirred at 80° C. for 16 hours. LCMS showed the reaction was complete. The reaction mixture was poured into water and extracted with EA. The combined organic layers were washed with brine, dried over Na₂SO₄ and concentrated. Purification by silica gel column chromatography (DCM/MeOH=10/1) provided compound 11-6 (320 mg, 55% yield) as a yellow oil. LCMS: Rt: 0.96 min; MS m/z (ESI): 427.3 [M+H]⁺.

### Step 8: Preparation of Compound 11

To a solution of compound 11-5 (175 mg, 0.35 mmol, 1.0 eq.) and compound 11-6 (150 mg, 0.35 mmol, 1.0 eq.) in THF (10 mL) were added DIPEA (136 mg, 1.05 mmol, 3.0 eq.) and NaI (10 mg, 0.07 mmol, 0.2 eq.). The mixture was stirred at 70° C. for 16 hours. LCMS showed the reaction was completed. The mixture was concentrated and purified by prep-HPLC to provide compound 11 (34 mg, 11% yield) as a yellow oil.

¹H NMR (400 MHz, CDCl₃) δ: 0.86-0.90 (m, 12H), 1.26-1.34 (m, 64H), 1.41-1.54 (m, 6H), 1.59-1.77 (m, 6H), 1.99-2.07 (m, 2H), 2.17-2.21 (m, 4H), 2.47-2.71 (m, 10H), 3.15-3.18 (m, 4H), 3.55-3.62 (m, 2H), 5.73-5.84 (m, 2H). LCMS: Rt: 1.610 min; MS m/z (ESI): 889.8 [M+H]⁺.

The following compounds were prepared in analogous fashion as Compound 11, using corresponding starting material.

| Compound | Characterization |
|---|---|
| 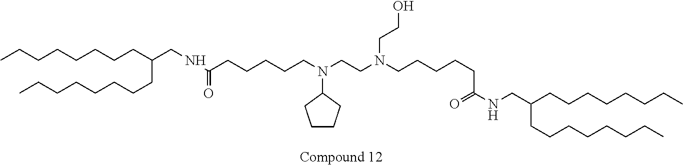 [SMILES: CCCCCCCCC(CCCCCCCC)CNC(=O)CCCCCN(CCO)CCN(CCCCCC(=O)NCC(CCCCCCCC)CCCCCCCC)C1CCCC1] | ¹H NMR (400 MHz, CDCl₃) δ: 0.86-0.90 (m, 12H), 1.26-1.32 (m, 64H), 1.36-1.57 (m, 8H), 1.63-1.68 (m, 6H), 1.75-1.86 (m, 2H), 2.16-2.20 (m, 4H), 2.45-2.70 (m, 10H), 3.15-3.18 (m, 4H), 3.52-3.59 (m, 2H), 5.68-5.81 (m, 2H). LCMS: Rt: 1.810 min; MS m/z (ESI): 904.8 [M + H]⁺. |
| 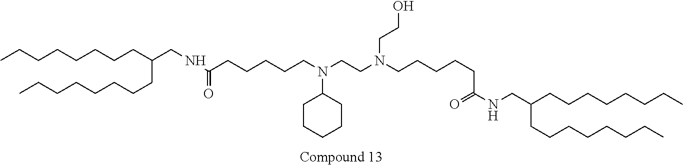 [SMILES: CCCCCCCCC(CCCCCCCC)CNC(=O)CCCCCN(CCO)CCN(CCCCCC(=O)NCC(CCCCCCCC)CCCCCCCC)C1CCCCC1] | ¹H NMR (400 MHz, CDCl₃) δ: 0.86-0.90 (m, 12H), 1.26-1.32 (m, 68H), 1.41-1.53 (m, 5H), 1.64-1.69 (m, 6H), 1.76-1.90 (m, 3H), 2.15-2.20 (m, 4H), 2.39-2.72 (m, 10H), 3.15-3.18 (m, 4H), 3.53-3.61 (m, 2H), 5.72-5.86 (m, 2H). LCMS: Rt: 1.680 min; MS m/z (ESI): 917.8 [M + H]⁺_{.} |
| 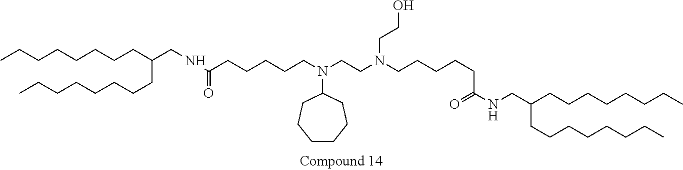 [SMILES: CCCCCCCCC(CCCCCCCC)CNC(=O)CCCCCN(CCO)CCN(CCCCCC(=O)NCC(CCCCCCCC)CCCCCCCC)C1CCCCCC1] | ¹H NMR (400 MHz, CDCl₃) δ: 0.86-0.90 (m, 12H), 1.26-1.32 (m, 64H), 1.36-1.60 (m, 12H), 1.65-1.70 (m, 6H), 1.83-1.89 (m, 2H), 2.16-2.20 (m, 4H), 2.41-2.72 (m, 10H), 3.13-3.19 (m, 4H), 3.55-3.57 (m, 2H), 5.66-5.79 (m, 2H). LCMS: Rt: 1.650 min; MS m/z (ESI): 931.8 [M + H]⁺. |

### 6.9 Example 9: Preparation of Compound 15

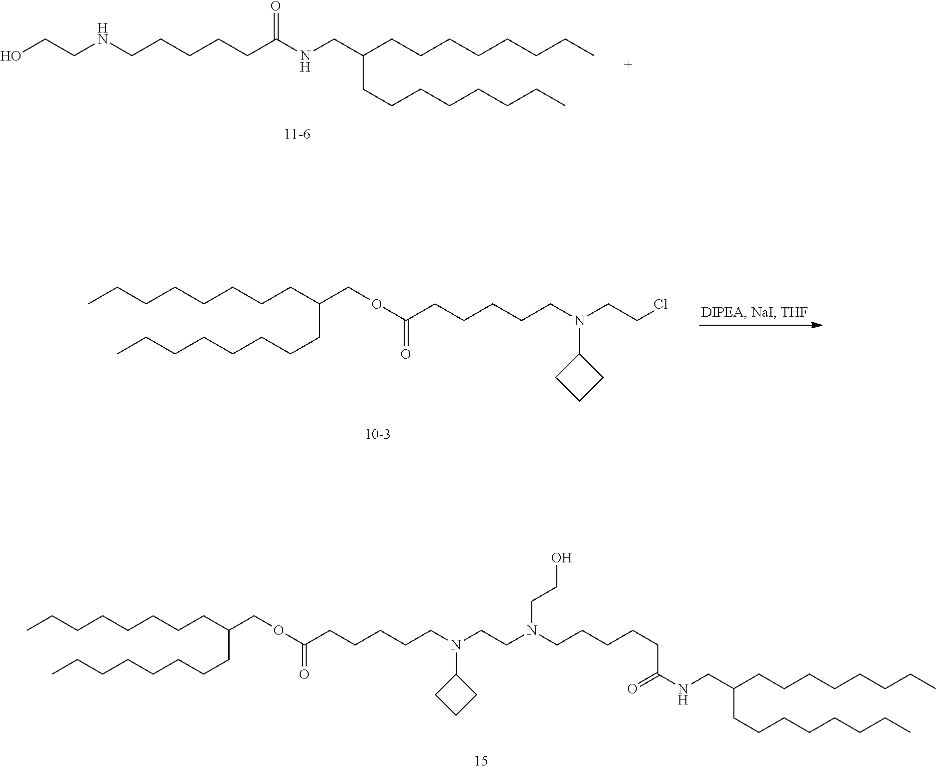

To a solution of compound 11-6 (221 mg, 0.52 mmol, 1.0 eq.) and compound 10-3 (259 mg, 0.52 mmol, 1.0 eq.) in THF (10 mL) were added DIPEA (202 mg, 1.56 mmol, 3.0 eq.) and NaI (16 mg, 0.104 mmol, 0.2 eq.). The mixture was stirred at 70° C. for 16 hours. LCMS showed the reaction was completed. The mixture was concentrated and purified by prep-HPLC to provide compound 15 (121 mg, 26% yield) as a yellow oil.

¹H NMR (400 MHz, CDCl₃) δ: 0.86-0.92 (m, 12H), 1.26-1.30 (m, 67H), 1.46-1.72 (m, 12H), 1.98-2.09 (m, 2H), 2.15-2.19 (m, 2H), 2.31-2.71 (m, 8H), 3.16-3.23 (m, 2H), 3.56-3.66 (m, 2H), 3.95-4.03 (m, 2H), 7.30 (s, 1H). LCMS: Rt: 1.68 min; MS m/z (ESI): 890.7 [M+H]⁺.

The following compounds were prepared in analogous fashion as Compound 15, using corresponding starting material.

| Compound | Characterization |
|---|---|
| 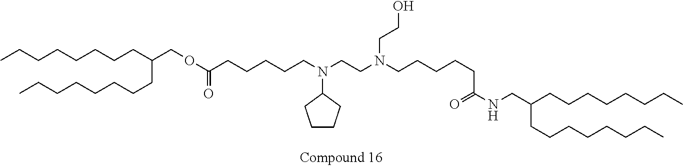 [SMILES: CCCCCCCCC(CCCCCCCC)CNC(=O)CCCCCN(CCO)CCN(CCCCCC(=O)OCC(CCCCCCCC)CCCCCCCC)C1CCCC1] | ¹H NMR (400 MHz, CDCl₃) δ: 0.82-0.92 (m, 12H), 1.16-1.34 (m, 58H), 1.35-1.51 (m, 4H), 1.56-1.94 (m, 24H), 1.96-2.12 (m, 4H), 2.18-2.26 (m, 2H), 2.31-2.38 (m, 2H), 3.02-3.12 (m, 2H), 3.13-3.21 (m, 2H), 3.92- 4.01 (m, 2H). LCMS: Rt: 1.820 min; MS m/z (ESI): 904.8 [M + H]⁺. |
| 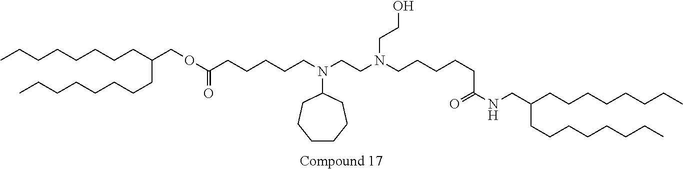 [SMILES: CCCCCCCCC(CCCCCCCC)CNC(=O)CCCCCN(CCO)CCN(CCCCCC(=O)OCC(CCCCCCCC)CCCCCCCC)C1CCCCCC1] | ¹H NMR (400 MHz, CDCl₃) δ: 0.82-0.94 (m, 12H), 1.17-1.42 (m, 64H), 1.46-1.72 (m, 15H), 1.75-1.83 (m, 2H), 1.86-1.92 (m, 2H), 1.97-2.04 (m, 2H), 2.16- 2.26 (m, 5H), 2.29-2.37 (m, 2H), 2.56-2.63 (m, 1H), 2.69-2.78 (m, 1H), 2.92-3.08 (m, 4H), 3.13- 3.20 (m, 2H), 3.35-3.46 (m, 1H), 3.65-3.74 (m, 1H), 3.92-4.01 (m, 2H). LCMS: Rt: 2.220 min; MS |
| | m/z (ESI): 932.8 [M + H]⁺. |
| 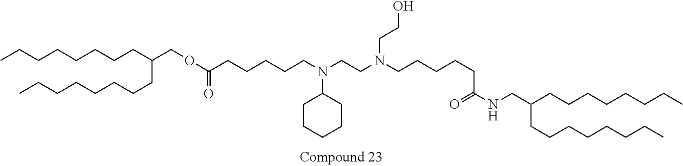 [SMILES: CCCCCCCCC(CCCCCCCC)CNC(=O)CCCCCN(CCO)CCN(CCCCCC(=O)OCC(CCCCCCCC)CCCCCCCC)C1CCCCC1] | ¹H NMR (400 MHz, CDCl₃) δ: 0.82-0.94 (m, 12H), 1.12-1.38 (m, 60H), 1.49-1.69 (m, 6H), 1.73-2.07 (m, 16H), 2.18-2.25 (m, 1H), 2.26-2.37 (m, 2H), 2.4- 2.6 (m, 10H), 3.38-3.53 (m, 2H), 3.54-3.79 (m, 2H), 3.96 (d, J = 5.6 Hz, 2H). LCMS: Rt: 1.890 min; MS m/z (ESI): 918.8 [M + H]⁺. |

### 6.10 Example 10: Preparation of Compound 18

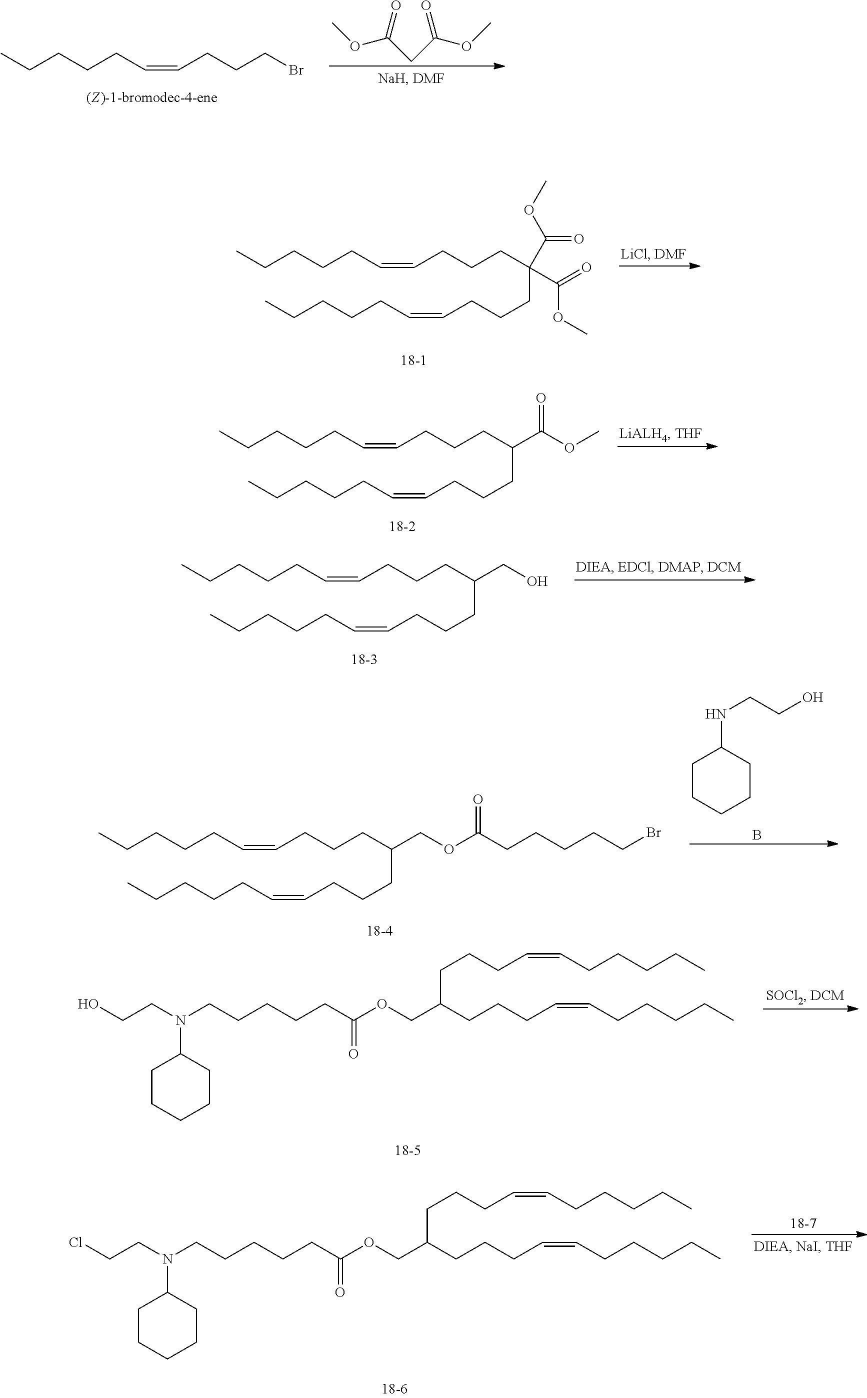

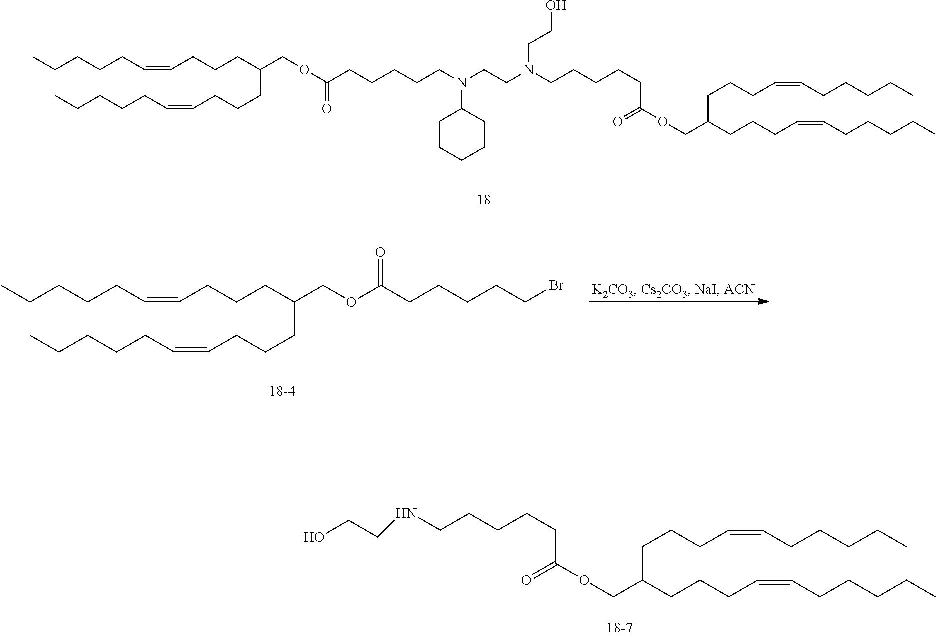

### Step 1: Preparation of Compound 18-1

To a stirred solution of dimethyl malonate (5 g, 38 mmol, 1 eq.) in DMF (76 mL) was added sodium hydride (3.8 g, 95 mmol, 2.5 eq.) at room temperature under argon atmosphere. After 0.5 h, (Z)-1-bromodec-4-ene (21 g, 95 mmol, 2.5 eq.) was added to the mixture, the mixture was stirred overnight at room temperature. The mixture was quenched with water (130 mL), extracted with EA (3×65 mL); the combined organic layer was washed with brine (2×65 mL), dried over anhydrous sodium sulfate, concentrated under vacuum. Purification by silica gel column chromatography (EA:PE=0%-5%) provided compound 18-1 (10.5 g, 68.2% yield) as colorless oil.

### Step 2: Preparation of Compound 18-2

A mixture of compound 18-1 (10.5 g, 25.7 mmol, 1 eq.) and LiCl (10.9 g, 257 mmol, 10 eq.) in DMF (180 mL) was stirred for 24 hours at 120° C. The mixture was diluted with water, extracted with EA, washed with brine, dried and concentrated. The residual was purified by silica gel column chromatography (EA:PE=0%-5%) to provide compound 18-2 (7.5 g, 83.2% yield) as colorless oil. ¹H NMR (400 MHz, CDCl₃) δ: 0.80-0.95 (m, 6H), 1.18-1.37 (m, 16H), 1.40-1.52 (m, 2H), 1.54-1.66 (m, 3H), 1.90-2.08 (m, 7H), 2.24-2.41 (m, 1H), 3.60-3.75 (m, 3H), 5.24-5.49 (m, 4H).

### Step 3: Preparation of Compound 18-3

A mixture of compound 18-2 (7.5 g, 21.5 mmol, 1 eq.), LiAlH₄ (1.6 g, 43 mmol, 2 eq.) in THF (100 mL) was stirred overnight at 80° C. The mixture was quenched with water, filtered; the filtrate was concentrated, purified by silica gel column chromatography (EA:PE=0% to 5%) to provide compound 18-3 (6.2 g, 89.8% yield) as a yellow oil.

### Step 4: Preparation of Compound 18-4

A mixture of compound 18-3 (1.8 g, 5.5 mmol, 1 eq.), 6-bromohexanoic acid (1.3 g, 6.6 mmol, 1.2 eq.), DIEA (2.14 g, 16.5 mmol, 3 eq.), DMAP (337 mg, 2.76 mmol, 0.5 eq.) and EDCI (1.27 g, 6.6 mmol, 1.2 mmol) in DCM (20 mL) was stirred overnight at 40° C. The mixture was concentrated and purified by silica gel column chromatography (EA:PE=0%-2%) to provide compound 18-4 (2.1 g, 75.2% yield) as colorless oil.

¹H NMR (400 MHz, CDCl₃) δ: 0.83-0.93 (m, 6H), 1.23-1.40 (m, 20H), 1.41-1.54 (m, 2H), 1.62-1.72 (m, 3H), 1.83-2.10 (m, 10H), 2.25-2.46 (m, 2H), 3.18-3.52 (m, 2H), 3.87-4.03 (m, 2H), 5.18-5.58 (m, 4H).

### Step 5: Preparation of Compound 18-5

A mixture of compound 18-4 (300 mg, 0.6 mmol, 1 eq.), compound B (133 mg, 0.9 mmol, 1.5 eq.), DIEA (232 mg, 1.8 mmol, 3 eq.) and sodium iodide (30 mg, 0.2 mmol, 0.3 eq.) in THF (6 mL) was stirred overnight at 70° C. The mixture was concentrated and purified by silica gel column chromatography (MeOH:DCM=0% to 10%) to provide compound 18-5 (147 mg, 43.6% yield) as colorless oil. LCMS: Rt: 0.900 min; MS m/z (ESI): 562.4 [M+H]⁺.

### Step 6: Preparation of Compound 18-6

A mixture of compound 18-5 (147 mg, 0.26 mmol, 1 eq.) and SOCl₂ (93 mg, 0.78 mmol, 3 eq.) in DCM (5 mL) was stirred overnight at 35° C. The mixture was concentrated to provide compound 18-6 (137 mg, 90.2% yield). LCMS: Rt: 1.210 min; MS m/z (ESI): 580.4 [M+H]⁺.

### Step 7: Preparation of Compound 18-7

A mixture of compound 18-4 (1971 mg, 2 mmol, 1 eq.), 2-aminoethanol (147 mg, 2.4 mmol, 1.2 mmol), K₂CO₃ (828 mg, 6 mmol, 3 eq.), Cs₂CO₃ (20 mg, 0.06 mmol, 0.03 eq.) and NaI (15 mg, 0.1 mmol, 0.05 eq.) in ACN (40 mL) was stirred overnight at 80° C. The mixture was concentrated and purified by silica gel column chromatography (MeOH:DCM=0% to 10%) to provide compound 18-7 (610 mg, 65.4% yield) as brown oil. LCMS: Rt: 0.910 min; MS m/z (ESI): 480.4 [M+H]⁺.

### Step 8: Preparation of Compound 18

A mixture of compound 18-6 (137 mg, 0.24 mmol, 1 eq.), compound 18-7 (138 mg, 0.29 mmol, 1.2 eq.), sodium iodide (10 mg, 0.07 mmol, 0.3 eq.) and DIEA (93 mg, 0.72 mmol, 3 eq.) in THF (5 mL) was stirred overnight at 70° C. The mixture was concentrated under vacuum. Purification by prep-HPLC provided compound 18 (21 mg, 8.7% yield) as light brown oil.

¹H NMR (400 MHz, CDCl₃) δ: 0.83-0.92 (m, 12H), 1.15-1.23 (m, 3H), 1.24-1.36 (m, 47H), 1.37-1.52 (m, 5H), 1.56-1.69 (m, 12H), 1.71-1.79 (m, 4H), 1.95-2.05 (m, 14H), 2.21-2.33 (m, 4H), 2.42-2.60 (m, 9H), 3.49-3.56 (m, 1H), 3.95-3.99 (m, 3H), 5.30-5.42 (m, 8H). LCMS: Rt: 0.640 min; MS m/z (ESI): 1023.7 [M+H]⁺.

### 6.11 Example 11: Preparation of Compound 19

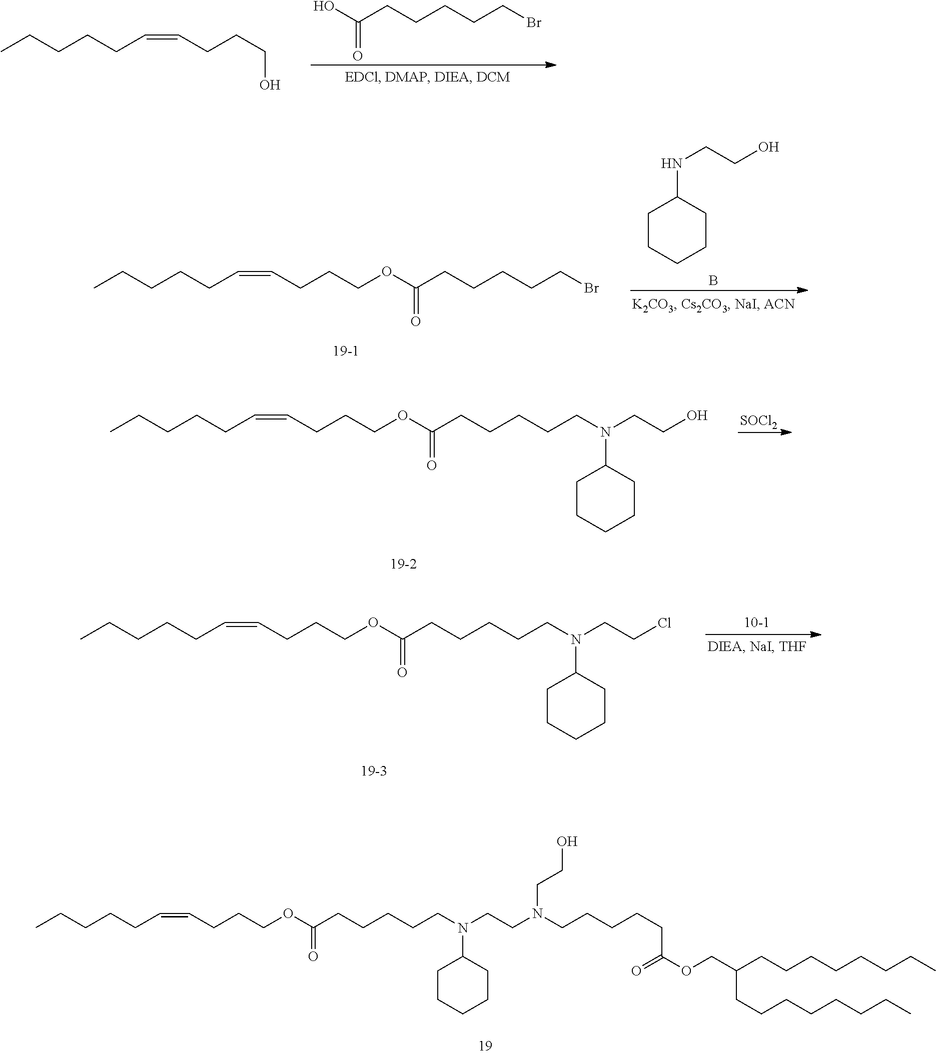

### Step 1: Preparation of Compound 19-1

To a solution of cis-4-decen-1-ol (1.56 g, 10.0 mmol, 1.0 eq.) and 6-bromohexanoic acid (2.9 g, 15.0 mmol, 1.5 eq.) in 30 mL of dichloromethane were added DIEA (3.87 g, 30.0 mmol, 3.0 eq.) and DMAP (244.0 mg, 2.0 mmol, 0.2 eq.). After stirring for 5 min at ambient temperature, EDCI (2.86 g, 15.0 mmol, 1.5 eq.) was added and the reaction mixture was stirred at room temperature overnight after which the TLC showed complete disappearance of the starting alcohol. The reaction mixture was diluted with dichloromethane (300 mL) and washed with saturated NaHCO₃ (100 mL), water (100 mL) and brine (100 mL). The combined organic layers were dried over Na₂SO₄ and solvents were removed in vacuo. Evaporation of the solvent gave the crude product which was purified by silica gel column chromatography (0-2% EA in PE) to provide compound 19-1 (1.3 g, 39%) as a colorless oil.

### Step 2: Preparation of Compound 19-2

To a solution of compound 19-1 (664.0 mg, 2.0 mmol, 1.0 eq.) and compound B (572.0 mg, 4.0 mmol, 2.0 eq.) in ACN (10.0 mL) were added Cs₂CO₃ (195.0 mg, 0.6 mmol, 0.3 eq.), K₂CO₃ (828.0 mg, 6.0 mmol, 3.0 eq.) and NaI (28.0 mg, 0.2 mmol, 0.1 eq.) at RT. The mixture was stirred for 16 hours at 85° C. LCMS showed the reaction was completed, the mixture was evaporated under reduced pressure and purified by FCC (DCM/MeOH=1/0-20/1) to provide compound 19-2 (0.37 g, 47% yield) as a yellow oil. LCMS: Rt: 0.740 min; MS m/z (ESI): 396.3 [M+H]⁺.

### Step 3: Preparation of Compound 19-3

To a solution of compound 19-2 (170.0 mg, 0.43 mmol, 1.0 eq.) in DCM (5.0 mL) was added SOCl₂ (152.0 mg, 1.29 mmol, 3.0 eq.) at RT. The mixture was stirred for 16 hours. LCMS showed the reaction was completed, concentration under reduced pressure provided compound 19-3 (0.2 g, crude) as brown oil. LCMS: Rt: 0.785 min; MS m/z (ESI): 414.3 [M+H]⁺.

### Step 4: Preparation of Compound 19

To a solution of compound 19-3 (200.0 mg, 0.48 mmol, 1.0 eq.) and compound 10-1 (247.0 mg, 0.58 mmol, 1.2 eq.) in THF (5.0 mL) was added DIEA (309.0 mg, 2.4 mmol, 5.0 eq.) at 0° C. The mixture was stirred for 16 hours at 70° C. LCMS showed the reaction was completed, concentration under reduced pressure and purification by prep-HPLC provided compound 19 (80.0 mg, 21% yield) as a yellow oil.

¹H NMR (400 MHz, CDCl₃) δ: 0.86-0.89 (m, 9H), 1.26-1.45 (m, 43H), 1.60-1.80 (m, 17H), 1.98-2.16 (m, 4H), 2.28-2.61 (m, 15H), 3.52-3.54 (m, 2H), 3.96-4.08 (m, 4H), 5.26-5.46 (m, 2H). LCMS: Rt: 1.137 min; MS m/z (ESI): 805.7 [M+H]⁺.

### 6.12 Example 12: Preparation of Compound 20

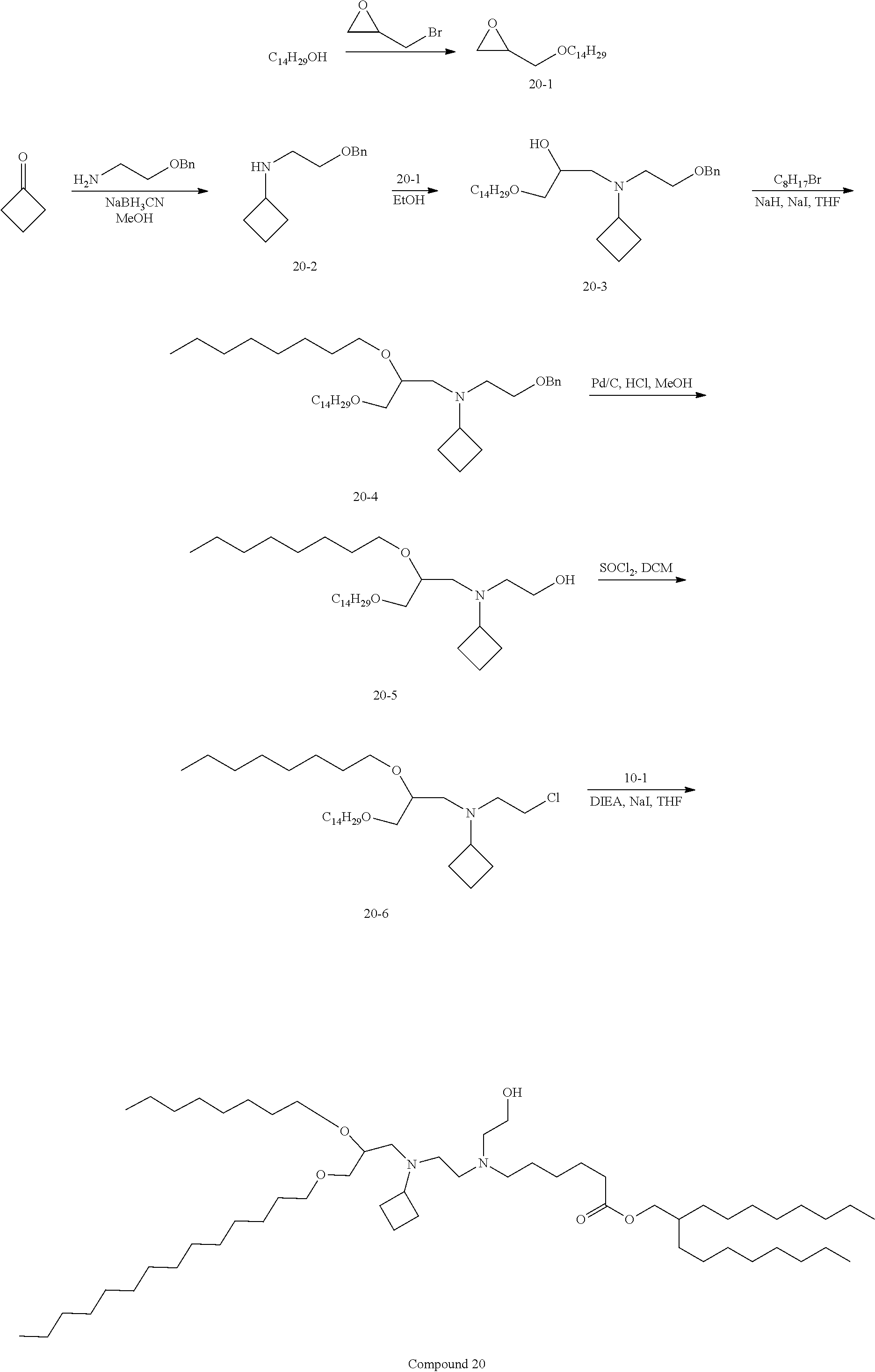

### Step 1: Preparation of Compound 20-1

To a solution of myristyl alcohol (2.1 g, 10.0 mmol, 1.0 eq.) in THF (20.0 mL) was added NaH (0.8 g, 20.0 mmol, 2.0 eq.). The mixture was stirred for 2 hours at RT, then 1-bromo-2,3-epoxypropane (2.5 g, 15.0 mmol, 1.5 eq.) was added and stirred for 16 h at 70° C. LCMS showed the reaction was completed, water was added, exacted with EA, concentrated and purified by FCC (PE/EA=20/1) to provide compound 20-1 (2.6 g, 96% yield) as colorless oil. ¹H NMR (400 MHz, CDCl₃) δ: 0.86-0.89 (m, 3H), 1.21-1.35 (m, 20H), 1.58-1.67 (m, 2H), 2.60-2.78 (m, 1H), 2.79-2.81 (m, 1H), 3.13-3.17 (m, 1H), 3.36-3.50 (m, 3H), 3.51-3.72 (m, 1H).

### Step 2: Preparation of Compound 20-2

To a solution of cyclobutanone (840 mg, 12.0 mmol, 1.2 eq.) in MeOH (10 mL) was added 2-(benzyloxy)ethan-1-amine (1.5 g, 10.0 mmol, 1.0 eq.). The mixture was stirred at 25° C. for 2 hours. Then to the mixture was added NaCNBH₃ (1.0 g, 15.0 mmol, 1.5 eq.). The mixture was stirred at 25° C. for 16 hours. LCMS showed the reaction was completed. The mixture was evaporated under reduced pressure and purified by FCC (DCM/MeOH=20/1) to provide compound 20-2 (1.0 g, crude) as a yellow oil.

### Step 3: Preparation of Compound 20-3

A solution of compound 20-1 (0.8 g, 2.96 mmol, 1.0 eq.) and compound 20-2 (1.0 g, 3.84 mmol, 1.3 eq.) in EtOH (10.0 mL) was stirred for 16 hours at 70° C. LCMS showed the reaction was completed. The reaction mixture was concentrated and purified by FCC (DCM/MeOH=30/1) to provide compound 20-3 (0.5 g, 35% yield) as a yellow oil. LCMS: Rt: 0.840 min; MS m/z (ESI): 476.3 [M+H]⁺.

### Step 4: Preparation of Compound 20-4

To a solution of compound 20-3 (475 mg, 1.0 mmol, 1.0 eq.) in THF (10.0 mL) was added NaH (160 mg, 4.0 mmol, 4.0 eq.). The mixture was stirred for 2 hours at RT, then C₈H₁₇Br (576 mg, 3.0 mmol, 3.0 eq.) was added and stirred for 16 hours at 70° C. LCMS showed the reaction was completed, water was added, exacted with EA, concentrated and purified by FCC (PE/EA=20/1) to provide compound 20-4 (300 mg, 51% yield) as colorless oil. LCMS: Rt: 1.280 min; MS m/z (ESI): 588.4 [M+H]⁺.

### Step 5: Preparation of Compound 20-5

To a solution of compound 20-4 (250 mg, 0.43 mmol, 1.0 eq.) in EA (10 mL) was added Pd/C (25.0 mg) and HCl (5 drops). The mixture was stirred for 16 h at RT under H₂. LCMS showed the reaction was completed, filtered and concentrated to provide compound 20-5 (250 mg, crude) as a yellow oil. LCMS: Rt: 1.023 min; MS m/z (ESI): 498.4 [M+H]⁺.

### Step 6: Preparation of Compound 20-6

To a solution of compound 20-5 (240 mg, 0.5 mmol, 1.0 eq.) in DCM (5.0 mL) was added SOCl₂ (177.0 mg, 1.5 mmol, 3.0 eq.) at RT. The mixture was stirred for 16 hours. LCMS show the reaction was completed, the mixture was concentrated under reduced pressure to provide compound 20-6 (0.27 g, crude) as brown oil.

### Step 7: Preparation of Compound 20

To a solution of compound 20-6 (120.0 mg, 0.23 mmol, 1.0 eq.) and compound 10-1 (120.0 mg, 0.28 mmol, 1.2 eq.) in THF (5.0 mL) was added DIEA (148.0 mg, 1.1 mmol, 5.0 eq.) at 0° C. The mixture was stirred for 16 hours at 70° C. LCMS showed the reaction was completed, the mixture was evaporated under reduced pressure and purified by prep-HPLC to provide compound 20 (30.0 mg, 14% yield) as a yellow oil.

¹H NMR (400 MHz, CDCl₃) δ: 0.86-0.89 (m, 12H), 1.21-1.35 (m, 65H), 1.50-1.65 (m, 11H), 1.98-2.00 (m, 3H), 2.28-2.32 (m, 2H), 2.53-2.62 (m, 9H), 3.40-3.59 (m, 10H), 3.96 (d, J=5.6 Hz, 2H). LCMS: Rt: 4.600 min; MS m/z (ESI): 907.8 [M+H]⁺.

### 6.13 Example 13: Preparation of Compound 22

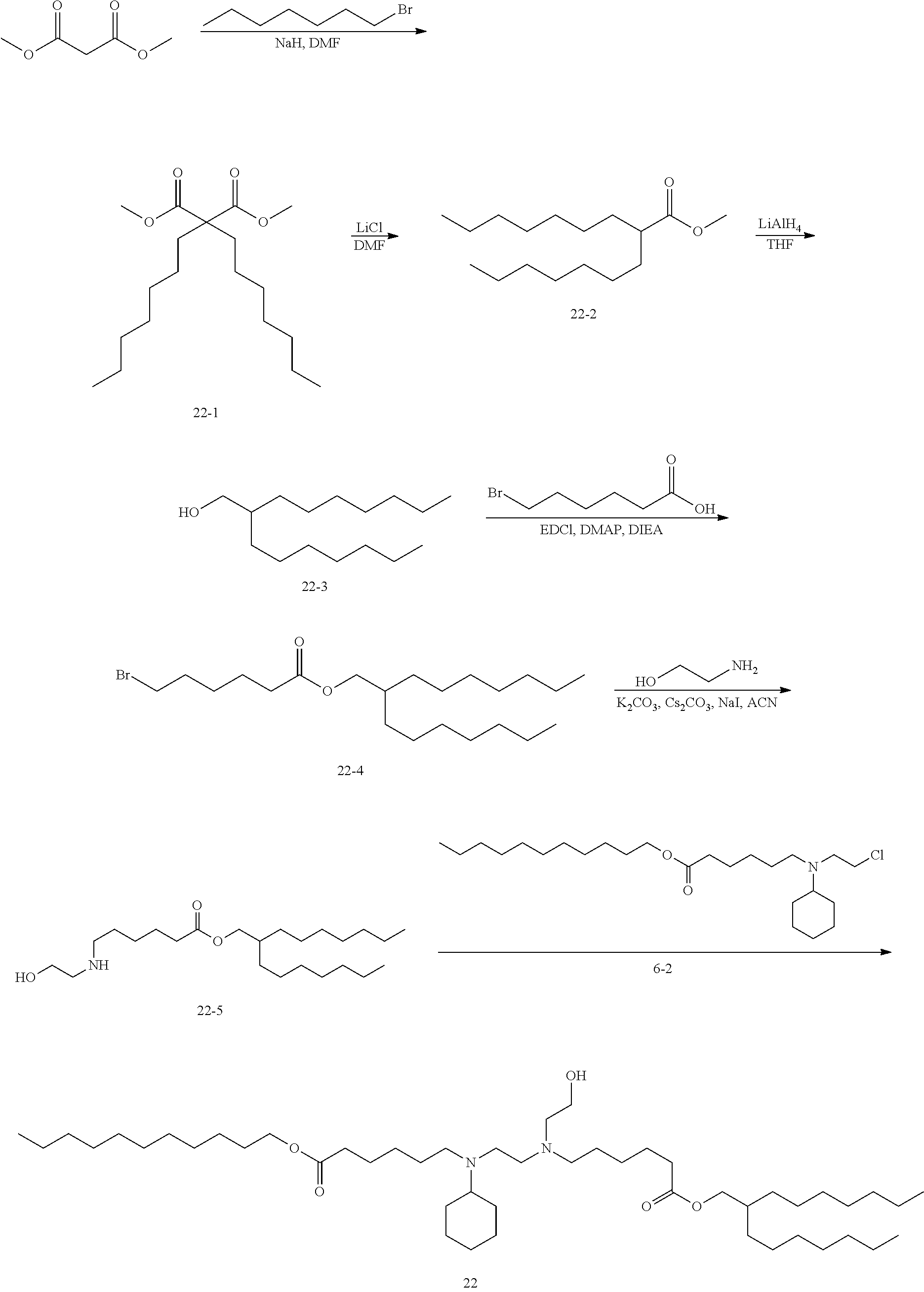

### Step 1: Preparation of Compound 22-1

To a mixture of NaH (3 g, 74.07 mmol, 2.5 eq.) in DMF (30 mL) was added dimethyl malonate (4 g, 30 mmol, 1.0 eq.) at 0° C. under N₂. The reaction mixture was stirred at 0° C. for 0.5 hours. 1-Bromoheptane (13.4 g, 75 mmol, 2.5 eq.) in DMF (30 mL) was added. The reaction mixture was stirred at RT for 16 hours. TLC showed the reaction was complete. The reaction mixture was quenched by water and washed with EA. The organic layer was separated and dried over Na₂SO₄. Removal of solvent and purification by FCC provided compound 22-1 (5.3 g, 53.78%) as colorless oil. ¹H NMR (400 MHz, CDCl₃) δ: 3.71 (s, 6H), 1.88-1.84 (m, 4H), 1.31-1.26 (m, 16H), 1.14-1.10 (m, 4H), 0.89-0.86 (m, 6H).

### Step 2: Preparation of Compound 22-2

To a solution of compound 22-1 (5.3 g, 16.13 mmol, 1.0 eq.) in DMF (100 mL) was added LiCl (6.8 g, 161.3 mmol, 10.0 eq.). The reaction mixture was stirred at 120° C. for 12 hours. TLC showed the reaction was complete. The reaction mixture was quenched by water and washed with EA. The organic layer was separated and dried over Na₂SO₄. Removal of solvent and purification by FCC provided compound 22-2 (3.4 g, 78.07%) as colorless oil. ¹H NMR (400 MHz, CDCl₃) δ: 3.67 (s, 3H), 2.33-2.31 (m, 1H), 1.60-1.40 (m, 6H), 1.25 (s, 18H), 0.89-0.86 (m, 6H).

### Step 3: Preparation of Compound 22-3

To a solution of compound 22-2 (3.4 g, 12.57 mmol, 1.0 eq.) in THF (60 mL) was added slowly LiAlH₄ (955 mg, 25.14 mmol, 2.0 eq.) at 0° C. The reaction mixture was stirred at reflux for 1 hour. TLC showed the reaction was complete. After being cooled to 0° C., the mixture was quenched with successive addition of water (1.3 mL), 15% aq. NaOH (1.3 mL) and water (3.9 mL). The resulting mixture was diluted with EA and the precipitate was removed by filtration. The filtrate was concentrated under reduced pressure, the crude product was purified by FCC to provide compound 22-3 (2.3 g, 75.48%) as a yellow oil. ¹H NMR (400 MHz, CDCl₃) δ: 3.54 (d, J=5.6 Hz, 2H), 1.46-1.40 (m, 2H), 1.27 (s, 24H), 0.90-0.87 (m, 6H).

### Step 4: Preparation of Compound 22-4

To a solution of compound 22-3 (1 g, 4.125 mmol, 1.0 eq.) in DCM (15 mL) were added 6-bromohexanoic acid (0.966 g, 4.950 mmol, 1.2 eq.), EDCI (1.19 g, 6.188 mmol, 1.5 eq.), DMAP (101 mg, 0.8250 mmol, 0.2 eq.), and DIEA (1.07 g, 8.250 mmol, 2.0 eq.). The reaction mixture was stirred at 50° C. for 16 hours. TLC showed the reaction was complete. Removal of solvent and purification by FCC provided compound 22-4 (1 g, 57.79%) as a yellow oil.

### Step 5: Preparation of Compound 22-5

To a solution of compound 22-4 (0.33 g, 0.79 mmol, 1.0 eq.) in ACN (15 mL) were added ethanolamine (49 mg, 0.79 mmol, 1.0 eq.), K₂CO₃ (329 mg, 2.384 mmol, 3.0 eq.), Cs₂CO₃ (78 mg, 0.2384 mmol, 0.3 eq.), and NaI (6 mg, 0.0397 mmol, 0.05 eq.). The reaction mixture was stirred at 80° C. for 16 hours. LCMS showed the reaction was complete. Removal of solvent and purification by FCC provided compound 22-5 (280 mg, 47.73%) as a yellow oil.

### Step 4: Preparation of Compound 22

To a solution of compound 22-5 (230.0 mg, 0.53 mmol, 1.0 eq.) and compound 6-2 (257.0 mg, 0.64 mmol, 1.2 eq.) in THF (10.0 mL) was added DIEA (413.0 mg, 3.2 mmol, 5.0 eq.) at 0° C. The mixture was stirred for 16 hours at 70° C. LCMS showed the reaction was completed, the mixture was evaporated under reduced pressure and purified by prep-HPLC to provide compound 22 (100.0 mg, 24% yield) as colorless oil.

¹H NMR (400 MHz, CDCl₃) δ: 0.86-0.89 (m, 9H), 1.26-1.35 (m, 52H), 1.46-1.49 (m, 3H), 1.60-1.65 (m, 8H), 1.78 (s, 3H), 2.28-2.32 (m, 5H), 2.49-2.60 (m, 10H), 3.54 (s, 2H), 3.95-4.06 (m, 4H). LCMS: Rt: 1.250 min; MS m/z (ESI): 793.7 [M+H]⁺.

### 6.14 Example 14: Preparation of Compound 25

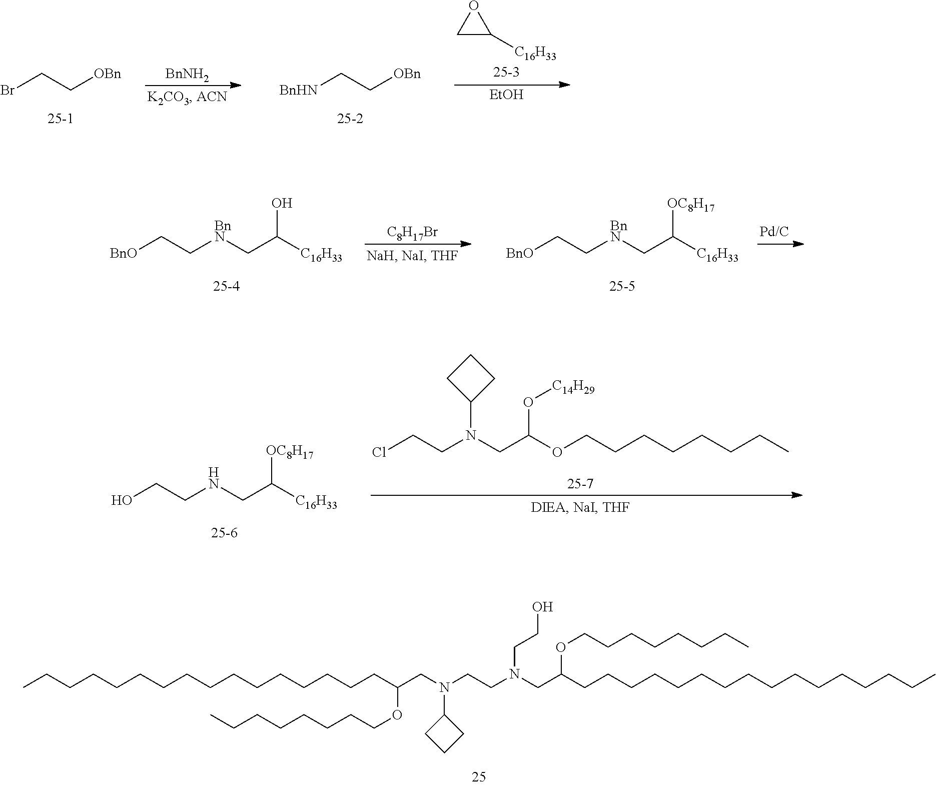

### Step 1: Preparation of Compound 25-2

To a mixture of compound 25-1 (5 g, 23.25 mmol, 1.0 eq.) in CH₃CN (200 mL) was added BnNH₂ (5 g, 46.5 mmol, 2.0 eq.) and K₂CO₃ (9.64 g, 69.75 mmol, 3.0 eq.). The reaction mixture was stirred at 80° C. for 10 hours. LCMS showed the reaction was completed. Removal of solvent, FCC to get the compound 25-2 (3.0 g, 53% yield) as colorless oil. LCMS: Rt: 0.740 min; MS m/z (ESI): 242.1 [M+H]⁺.

### Step 2: Preparation of Compound 25-4

To a mixture of compound 25-2 (2.5 g, 10.36 mmol, 1.0 eq.), compound 25-3 (5.56 g, 20.72 mmol, 2.0 eq.) in EtOH (100 mL) was stirred at 70° C. for 10 hours. LCMS showed the reaction was completed. Removal of solvent, FCC to get the compound 25-4 (2.5 g, 47% yield) as a yellow oil. LCMS: Rt: 1.320 min; MS m/z (ESI): 510.4 [M+H]⁺.

### Step 3: Preparation of Compound 25-5

To a mixture of NaH (710 mg, 17.65 mmol, 6.0 eq.) in THF (60 mL) was added compound 25-4 (1.5 g, 2.94 mmol, 1.0 eq.) at RT under N₂. The reaction mixture was stirred at RT for 2 hours. The C₈H₁₇Br (2.27 g, 11.77 mmol, 4.0 eq.) was added to it. The reaction mixture was stirred at 70° C. for 10 hours. LCMS showed the reaction was completed. The mixture power in water and washed with EA. The organic was separated and dried over Na₂SO₄. Removal of solvent, FCC to get the compound 25-5 (0.8 g, 43% yield) as a yellow oil. LCMS: Rt: 0.733 min; MS m/z (ESI): 622.5 [M+H]⁺.

### Step 4: Preparation of Compound 25-6

To a solution of compound 25-5 (0.8 g, 1.29 mmol, 1.0 eq.) in ethyl acetate (100 mL) was added Pd/C (1.0 g). The reaction mixture was stirred at RT for 48 hours under H₂. LCMS showed the reaction was completed. The mixture was filtered through diatomite. Removal of solvent to get the compound 25-6 (350 mg, 61% yield) as a yellow oil. LCMS: Rt: 1.040 min; MS m/z (ESI): 442.4 [M+H]⁺.

### Step 5: Preparation of Compound 25

To a mixture of compound 25-6 (350 mg, 0.8 mmol, 1.0 eq.), DIEA (200 mg, 1.6 mmol, 2.0 eq.) in THF (20 mL) was added compound 25-7 (200 mg, 0.4 mmol, 0.5 eq.), NaI (60 mg). The reaction mixture was stirred at 70° C. for 10 hours. LCMS showed the reaction was complete. After removal of solvent, the residue was purified by prep-HPLC to give the title compound (20 mg, 12% yield) as a yellow oil.

¹H NMR (400 MHz, CDCl₃) δ: 0.87 (t, J=8 Hz, 12H), 1.26-1.97 (m, 91H), 2.19-2.64 (m, 10H), 3.28-3.53 (m, 9H). LCMS: Rt: 0.627 min; MS m/z (ESI): 919.8 [M+H]⁺.

### 6.15 Example 15: Preparation of Compound 26

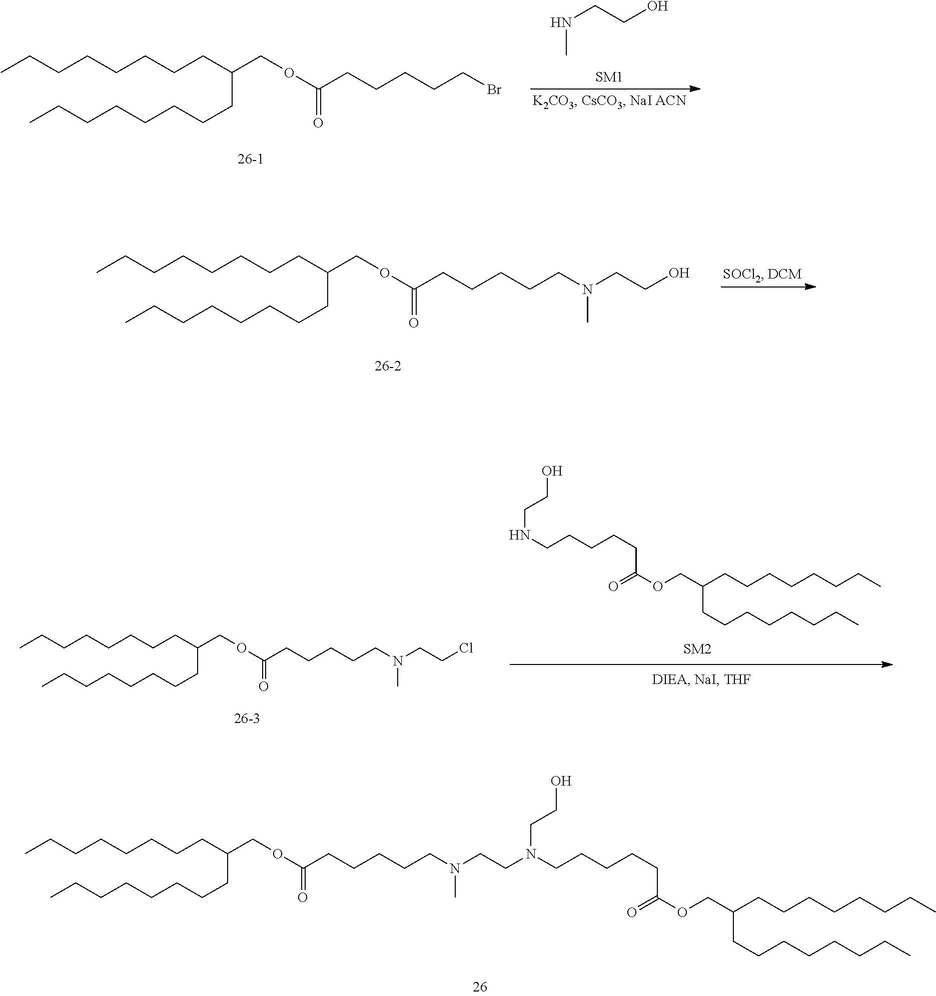

### Step 1: Preparation of Compound 26-2

To a solution of compound 26-1 (500 mg, 1.12 mmol, 1.0 eq.) and compound SM1 (170 mg, 2.24 mmol, 2.0 eq.) in ACN (10 mL) was added Cs₂CO₃ (95 mg, 0.34 mmol, 0.3 eq.), K₂CO₃ (465 mg, 3.36 mmol, 3.0 eq.) and NaI (14 mg, 0.1 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound 26-2 (500 mg, 81% yield) as a yellow oil. LCMS: Rt: 1.680 min; MS m/z (ESI): 442.4 [M+H]⁺.

### Step 2: Preparation of Compound 26-3

To a solution of compound 26-2 (100 mg, 0.23 mmol, 1.0 eq.) in DCM (10 mL) was added SOCl₂ (82 mg, 0.69 mmol, 3.0 eq.) at RT. The mixture was stirred for 16 hours at 35° C. LCMS show the reaction was completed, the mixture was evaporated under reduced pressure to provide compound 26-3 (100 mg, crude) as a yellow oil. LCMS: Rt: 0.920 min; MS m/z (ESI): 460.3 [M+H]⁺.

### Step 3: Preparation of Compound 26

To a solution of compound 26-3 (110 mg, 0.24 mmol, 1.0 eq.) and compound SM2 (100 mg, 0.24 mmol, 1.0 eq.) in THF (10 mL) was added DIEA (413 mg, 3.2 mmol, 5.0 eq.) and NaI (5 mg, 0.02 mmol, 0.1 eq.) at 0° C. The mixture was stirred for 16 hours at 70° C. LCMS show the reaction was completed, the mixture was evaporated under reduced pressured purified with pre-HPLC to provide compound 26 (20 mg, 10% yield) as colorless oil. ¹H NMR (400 MHz, CDCl₃) δ: 0.86-0.90 (m, 12H), 1.26-1.39 (m, 59H), 1.58-1.68 (m, 9H), 2.29-2.34 (m, 4H), 2.77-3.24 (m, 16H), 3.73 (s, 2H), 3.95-3.97 (m, 4H). LCMS: Rt: 1.760 min; MS m/z (ESI): 851.8 [M+H]⁺.

The following compounds were prepared in analogous fashion as Compound 26, using corresponding starting material.

| Compound | Characterization |
|---|---|
| 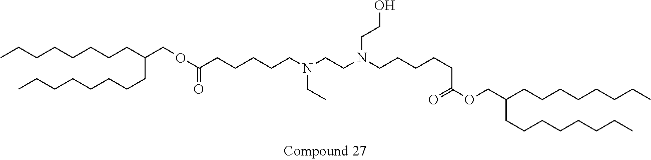 [SMILES: CCCCCCCCC(CCCCCCCC)COC(=O)CCCCCN(CC)CCN(CCO)CCCCCC(=O)OCC(CCCCCCCC)CCCCCCCC] | ¹H NMR (400 MHz, CDCl₃) δ: 0.83- 0.9 (m, 12H), 1.16-1.17 (m, 58H), 1.26-1.40 (m, 6H), 1.58-1.74 (m, 10H), 2.30-2.35 (m, 4H), 2.94-3.20 (m, 9H), 3.42-3.45 (m, 3H), 3.866- 3.97 (m, 2H), 5.30 (s, 4H). LCMS: Rt: 1.720 min; MS m/z (ESI): 865.8 [M + H]⁺. |
| 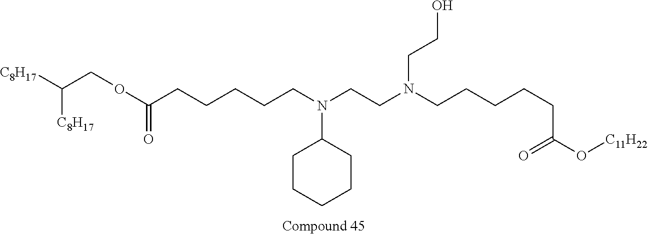 | ¹H NMR (400 MHz, CDCl₃) δ: 0.86- 0.95 (m, 9H), 1.18-1.36 (m, 55H), 1.45-1.59 (m, 4H), 1.60-1.65 (m, 8H), 1.70-1.80 (m, 4H), 2.28-2.34 (m, 4H), 2.48-2.60 (m, 10H), 3.47- 3.60 (m, 2H), 3.95-3.97 (m, 2H), 4.03-4.07 (m, 2H). LCMS: Rt: 1.190 min; MS m/z (ESI): 821.7 [M + H]⁺. |
| 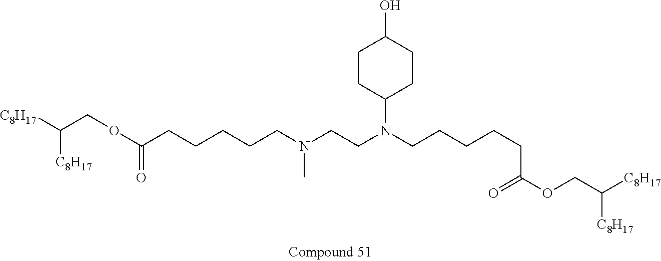 | ¹H NMR (400 MHz, CDCl₃) δ: 0.87 (t, J = 8 Hz, 12H), 1.26-2.01 (m, 79H), 2.24-2.55 (m, 16H), 3.54-3.56 (m, 1H), 3.95-3.97 (m, 4H). LCMS: Rt: 0.600 min; MS m/z (ESI): 905.7 [M + H]⁺. |
| 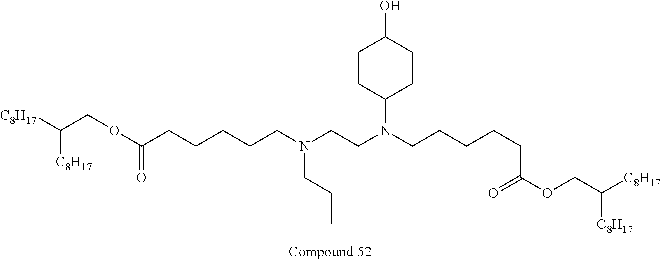 | ¹H NMR (400 MHz, CDCl₃) δ: 0.87 (t, J = 8 Hz, 15H), 1.04-2.01 (m, 81H), 2.28-2.47 (m, 15H), 3.53-3.56 (m, 1H), 3.96-3.97 (m, 4H). LCMS: Rt: 0.600 min; MS m/z (ESI): 933.7 [M + H]⁺. |
| 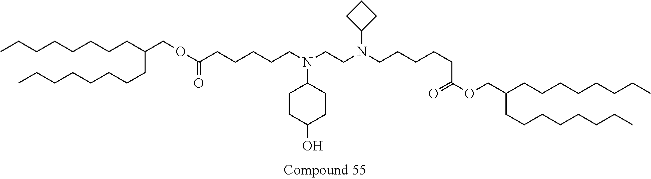 [SMILES: CCCCCCCCC(CCCCCCCC)COC(=O)CCCCCN(CCN(CCCCCC(=O)OCC(CCCCCCCC)CCCCCCCC)C1CCC(O)CC1)C1CCC1] | ¹H NMR (400 MHz, CDCl₃) δ: 0.87- 0.90 (m, 12H), 1.28-1.35 (m, 64H),1.36-1.46 (m, 5H), 1.63-1.64 (m, 8H), 1.76-1.86 (m, 4H), 1.94- 2.06 (m, 4H), 2.30-2.46 (m, 12H), 2.82-3.11 (m, 2H), 3.51-3.59 (m, 1H), 3.96-3.97 (m, 4H). LCMS: Rt: 1.99 min; MS m/z (ESI): 945.7 [M + H]⁺. |
| 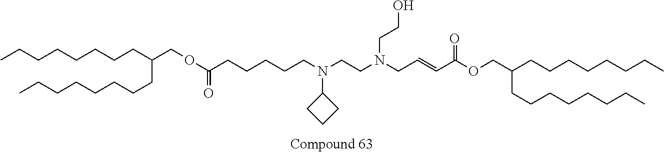 [SMILES: CCCCCCCCC(CCCCCCCC)COC(=O)/C=C/CN(CCO)CCN(CCCCCC(=O)OCC(CCCCCCCC)CCCCCCCC)C1CCC1] | ¹H NMR (400 MHz, CDCl₃) δ: 0.86- 0.90 (m, 12H), 1.26-1.38 (m, 60H), 1.43-1.48 (m, 2H), 1.55-1.71 (m, 6H), 1.85-2.03 (m, 3H), 2.26-2.32 (m, 2H), 2.40-2.45 (m, 2H), 2.54- 2.71 (m, 4H), 2.88-3.12 (m, 3H), 3.31-3.42 (m, 2H), 3.54-3.72 (m, 2H), 3.95-3.97 (m, 2H), 4.03-4.04 (m, 2H), 5.95-5.99 (m, 1H), 6.90- 6.98 (m, 1H). LCMS: Rt: |
| | 1.560 min; MS m/z (ESI): 861.6 |
| | [M + H]⁺. |
| 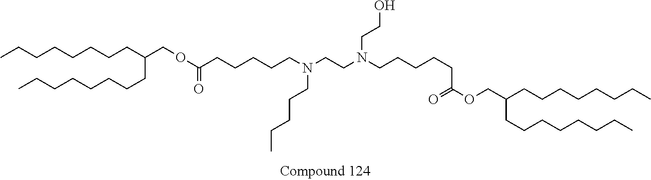 [SMILES: CCCCCCCCC(CCCCCCCC)COC(=O)CCCCCN(CCO)CCN(CCCCC)CCCCCC(=O)OCC(CCCCCCCC)CCCCCCCC] | ¹H NMR (400 MHz, CDCl₃) δ: 0.83- 0.94 (m, 12H), 1.26-1.35 (m,62H), 1.35-1.54 (m, 5H), 1.55-1.70 (m, 8H), 1.84-2.04 (m, 5H), 2.05-2.22 (m, 1H), 2.23-2.34 (m, 4H), 2.35- 2.66 (m, 9H), 2.84-3.11 (m, 2H), 3.47-3.68 (m, 2H), 3.93-4.02 (m, 4H). LCMS: Rt: 2.210 min; MS m/z (ESI): 907.8 [M + H]⁺. |
| 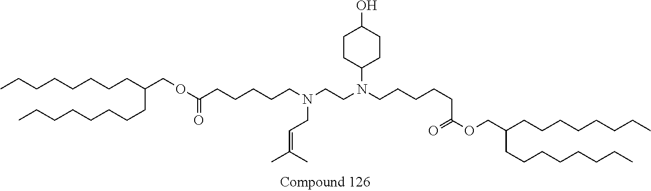 [SMILES: CCCCCCCCC(CCCCCCCC)COC(=O)CCCCCN(CC=C(C)C)CCN(CCCCCC(=O)OCC(CCCCCCCC)CCCCCCCC)C1CCC(O)CC1] | ¹H NMR (400 MHz, CDCl₃) δ: 0.79- 0.93 (m, 12H),1.11-1.36 (m, 73H), 1.62-1.67 (m, 6H), 1.69-1.84 (m, 5H), 1.93-2.09 (m, 2H), 2.21-2.34 (m, 4H), 2.35-2.58 (m, 8H), 2.95- 3.15 (m, 2H), 3.43-3.64 (m, 1H), 3.86-4.05 (m, 4H), 5.15-5.27 (m, 1H). LCMS: Rt: 2.150 min; MS m/z (ESI): 959.8 [M + H]⁺. |

### 6.16 Example 16: Preparation of Compound 28

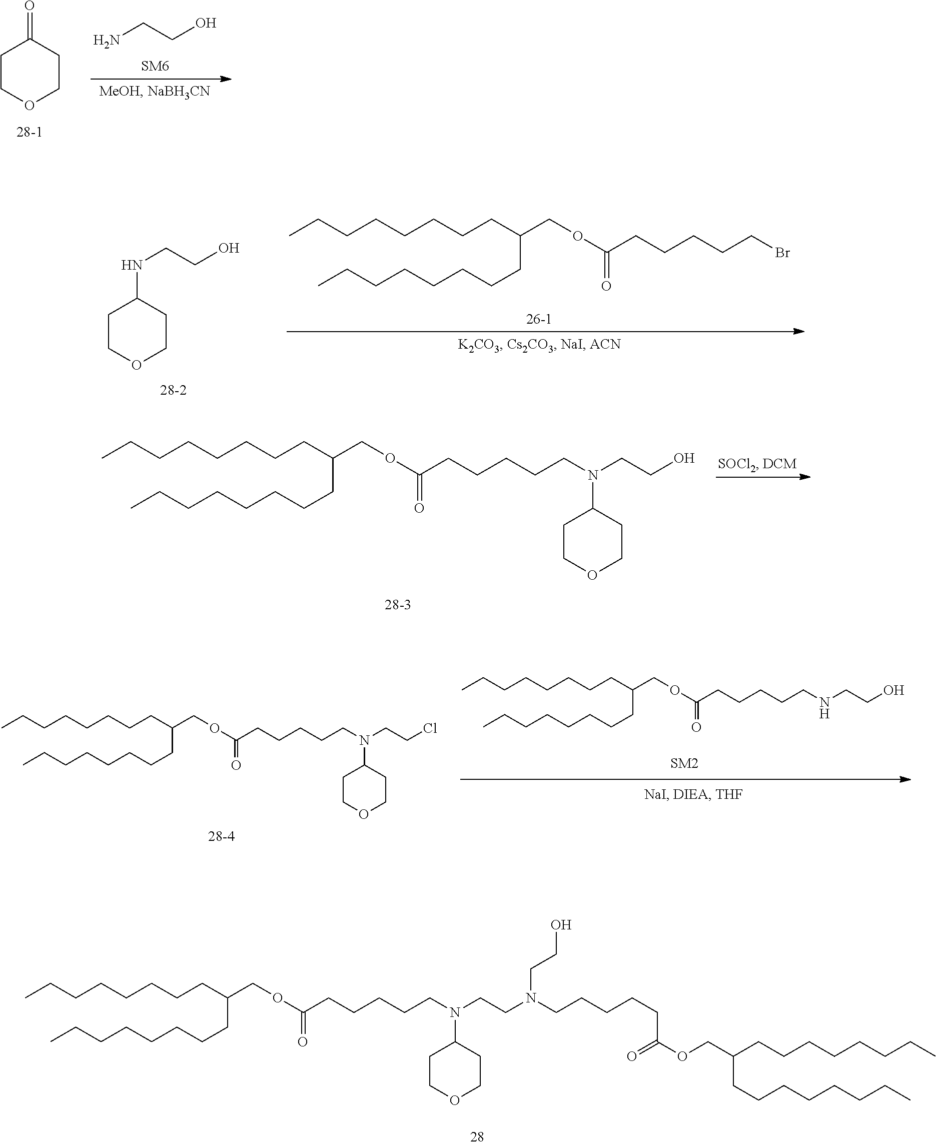

### Step 1: Preparation of Compound 28-2

A mixture of compound 28-1 (1 g, 10 mmol, 1.0 eq.), SM6 (0.9 g, 15 mmol, 1.5 eq.), two drops of AcOH in MeOH (20 mL) was stirred overnight at RT. The reagent of NaBH₃CN (0.9 g, 15 mmol, 1.5 eq.) was added to the mixture and stirred for an hour. The mixture was quenched with water, extracted over ethyl acetate, concentrated and purified by column chromatography silica gel (MeOH:DCM=0% to 10%) to give the desired product compound 28-2 (893 mg, 61.6% yield) as a yellow oil. LCMS: Rt: 0.380 min; MS m/z (ESI): 146.2 [M+H]⁺.

### Step 2: Preparation of Compound 28-3

A mixture of compound 26-1 (250 mg, 0.56 mmol, 1.0 eq.), compound 28-1 (243 mg, 1.68 mmol, 3.0 eq.), K₂CO₃ (232 mg, 1.68 mmol, 3.0 eq.), Cs₂CO₃ (7 mg, 0.02 mmol, 0.03 eq.) and sodium iodide (30 mg, 0.2 mmol, 0.3 eq.) in ACN (10 mL) was stirred overnight at 80° C. The mixture was concentrated and purified by column chromatography silica gel (MeOH:DCM=0% to 10%) to give the desired product compound 28-3 (122 mg, 42.7% yield) as a yellow oil. LCMS: Rt: 0.910 min; MS m/z (ESI): 512.4 [M+H]⁺.

### Step 3: Preparation of Compound 28-4

A mixture of compound 28-3 (122 mg, 0.24 mmol, 1.0 eq.) and SOCl₂ (85 mg, 0.72 mmol, 3.0 eq.) in DCM (5 mL) was stirred overnight at 35° C. The mixture was concentrated to give the desired product compound 28-4 (125 mg, crude) as a yellow oil. LCMS: Rt: 1.350 min; MS m/z (ESI): 530.4 [M+H]⁺.

### Step 4: Preparation of Compound 28

A mixture of compound 28-4 (125 mg, 0.24 mmol, 1.0 eq.), compound SM2 (100 mg, 0.23 mmol, 1.0 eq.), sodium iodides (20 mg, 0.13 mmol, 0.6 eq.) and DIEA (155 mg, 1.20 mmol, 5.0 eq.) in THF (5 mL) was stirred overnight at 70° C. The mixture was concentrated under vacuum. The residual was purified by prep-HPLC to give the desired product compound 28 (23 mg, 10.6% yield) as a yellow oil.

¹H NMR (400 MHz, CDCl₃) δ: 0.82-0.94 (m, 12H), 1.17-1.39 (m, 60H), 1.49-1.73 (m, 13H), 1.95-2.06 (m, 1H), 2.17-2.37 (m, 6H), 2.40-3.11 (m, 11H), 3.27-3.45 (m, 2H), 3.90-3.97 (m, 4H), 3.99-4.09 (m, 2H). LCMS: Rt: 2.240 min; MS m/z (ESI): 921.8 [M+H]⁺.

The following compounds were prepared in analogous fashion as Compound 28, using corresponding starting material.

| Compound | Characterization |
|---|---|
| 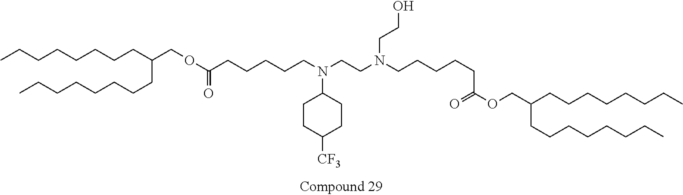 [SMILES: CCCCCCCCC(CCCCCCCC)COC(=O)CCCCCN(CCO)CCN(CCCCCC(=O)OCC(CCCCCCCC)CCCCCCCC)C1CCC(C(F)(F)F)CC1] | ¹H NMR (400 MHz, CDCl₃) δ: 0.86-0.90 (m, 12H), 1.26 (s, 65H), 1.45-1.47 (m, 4H), 1.60-1.67 (m, 7H), 1.89-2.01 (m, 4H), 2.28-2.32 (m, 4H),2.41-2.50 (m, 8H),2.59 (s, 2H), 3.51-3.54 (m, 2H), 3.97 (d, J = 5.6 Hz, 4H). LCMS: Rt: 0.560 min; MS m/z (ESI): 987.7 [M + H]⁺. |
| 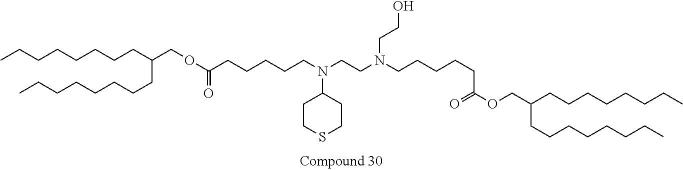 [SMILES: CCCCCCCCC(CCCCCCCC)COC(=O)CCCCCN(CCO)CCN(CCCCCC(=O)OCC(CCCCCCCC)CCCCCCCC)C1CCSCC1] | ¹H NMR (400 MHz, CDCl₃) δ: 0.86-0.90 (m, 12H), 1.26-1.33 (m, 60H), 1.43-1.48 (m, 4H), 1.60- 1.67 (m, 9H), 2.07-2.11 (m, 2H), 2.28-2.33 (m, 4H), 2.41-2.50 (m, 9H), 2.57-2.59 (m, 2H), 2.66-2.68 (m, 4H), 3.50-3.53 (m, 2H), 3.96- 3.98 (m, 4H). LCMS: Rt: 1.477 min; MS m/z (ESI): 937.7 [M + H]⁺. |
| 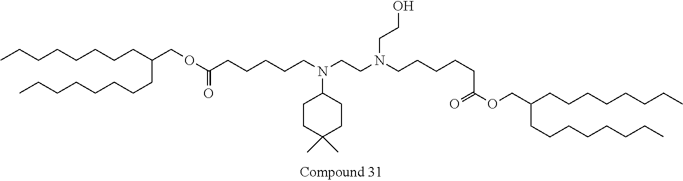 [SMILES: CCCCCCCCC(CCCCCCCC)COC(=O)CCCCCN(CCO)CCN(CCCCCC(=O)OCC(CCCCCCCC)CCCCCCCC)C1CCC(C)(C)CC1] | ¹H NMR (400 MHz, CDCl₃) δ: 0.86-0.90 (m, 18H), 1.15-1.32(m, 65H), 1.33-1.47 (m, 5H), 1.55- 1.68 (m, 9H), 2.28-2.32(m, 4H), 2.43-2.51 (m, 9H), 2.59 (s, 2H), 3.51-3.54 (m, 2H), 3.97 (d, J = 5.6 Hz, 4H). LCMS: Rt: 1.927 min; MS m/z (ESI): 947.8 [M + H]⁺. |
| 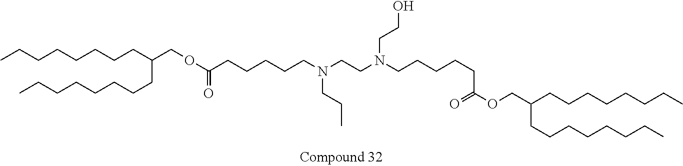 [SMILES: CCCCCCCCC(CCCCCCCC)COC(=O)CCCCCN(CCC)CCN(CCO)CCCCCC(=O)OCC(CCCCCCCC)CCCCCCCC] | ¹H NMR (400 MHz, CDCl₃) δ: 0.81-0.98 (m, 15H), 1.17-1.39 (m, 63H), 1.41-1.71 (m, 12H), 2.24- 2.37 (m, 6H), 2.48-2.74 (m, 7H), 3.48-3.70 (m, 2H), 3.91-4.03 (m, 4H). LCMS: Rt: 2.390 min; MS m/z (ESI): 879.8 [M + H]⁺. |
| 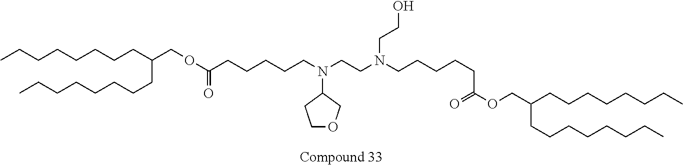 [SMILES: CCCCCCCCC(CCCCCCCC)COC(=O)CCCCCN(CCO)CCN(CCCCCC(=O)OCC(CCCCCCCC)CCCCCCCC)C1CCOC1] 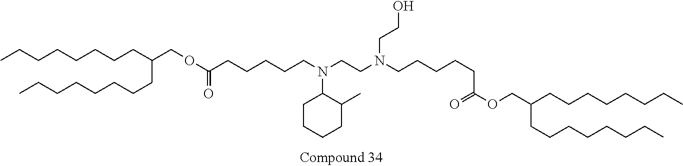 [SMILES: CCCCCCCCC(CCCCCCCC)COC(=O)CCCCCN(CCO)CCN(CCCCCC(=O)OCC(CCCCCCCC)CCCCCCCC)C1CCCCC1C] | ¹H NMR (400 MHz, CDCl₃) δ: 0.81-0.96 (m, 12H), 1.18-1.42 (m, 60H), 1.50-1.73 (m, 8H), 1.76- 2.16 (m, 4H), 2.20-2.39 (m, 6H), 2.42-3.25 (m, 10H), 3.60-4.07 (m, 9H). LCMS: Rt: 2.190 min; MS m/z (ESI): 908.7 [M + H]⁺. P1: ¹H NMR (400 MHz, CDCl₃) δ: 0.78-0.96 (m, 12H), 1.07-1.13 (m, 3H),1.19-1.47 (m, 66H), 1.54-1.78 (m, 12H), 1.82-2.06 (m, 3H), 2.18- 2.41 (m, 7H), 2.60-2.74 (m, 2H), 2.78-3.19 (m, 6H), 3.74 (s, 1H), 3.96 (d, J = 5.6 Hz, 4H). LCMS: Rt: 0.600 min; MS m/z (ESI): 933.8 [M + H]⁺. CAD: 45 min. P2: ¹H NMR (400 MHz, CDCl₃) δ: 0.81-0.92 (m, 12H), 0.93-1.01 (m, 3H), 1.07-1.43 (m, 65H), 1.47- 1.70 (m, 11H), 1.72-1.89 (m, 5H), |
| | 2.26-2.40 (m, 5H), 2.44-2.79 (m, |
| | 4H), 2.82-3.20 (m, 6H), 3.90-4.03 |
| | (m, 4H). LCMS: Rt: 0.600 min; |
| | MS m/z (ESI): 933.8 [M + H]⁺. |
| | CAD: 69 min. |
| 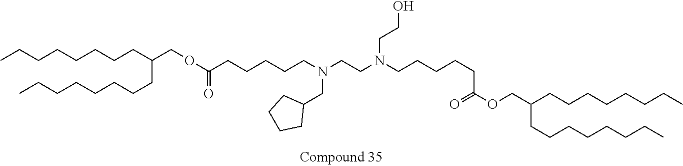 [SMILES: CCCCCCCCC(CCCCCCCC)COC(=O)CCCCCN(CCO)CCN(CCCCCC(=O)OCC(CCCCCCCC)CCCCCCCC)CC1CCCC1] | ¹H NMR (400 MHz, CDCl₃) δ: 0.81-0.96 (m, 12H), 1.10-1.42 (m, 61H), 1.43-1.71 (m, 14H), 1.74- 1.93 (m, 2H), 2.24-2.38 (m, 6H), 2.42-3.25 (m, 12H), 3.47-3.80 (m, 2H), 3.96 (d, J = 5.2 Hz, 4H). LCMS: Rt: 0.600 min; MS m/z (ESI): 919.8 [M + H]⁺. |
| 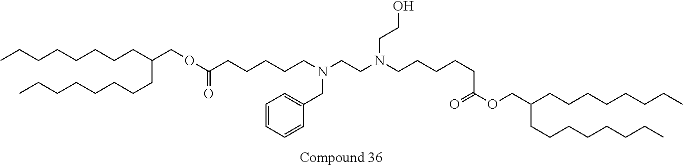 [SMILES: CCCCCCCCC(CCCCCCCC)COC(=O)CCCCCN(CCO)CCN(CCCCCC(=O)OCC(CCCCCCCC)CCCCCCCC)Cc1ccccc1] | ¹H NMR (400 MHz, CDCl₃) δ: 0.80-0.97 (m, 12H), 1.15-1.36 (m, 62H), 1.37-1.66 (m, 10H), 2.18- 2.34 (m, 4H), 2.36-2.82 (m, 10H), 3.44-3.71 (m, 3H), 3.91-4.04 (m, 4H), 7.31-7.48 (m, 5H). LCMS: Rt: 0.627 min; MS m/z (ESI): 927.7 [M + H]⁺. |
| 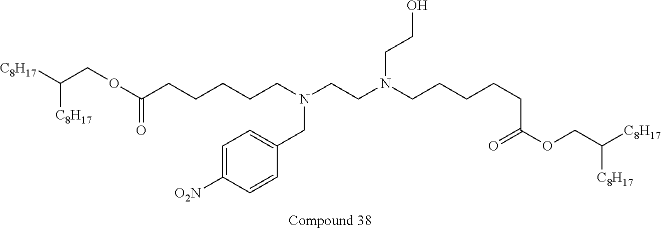 | ¹H NMR (400 MHz, CDCl₃) δ: 0.87 (t, J = 8 Hz, 12H), 1.26-1.43 (m, 71H), 2.25-2.57 (m, 14H), 3.49-3.52 (m, 2H), 3.65-3.67 (m, 2H), 3.95-3.97 (m, 4H), 7.52 (d, J = 8 Hz, 2H), 8.17 (d, J = 8 Hz, 2H). LCMS: Rt: 0.627 min; MS m/z (ESI): 972.7 [M + H]⁺. |
| 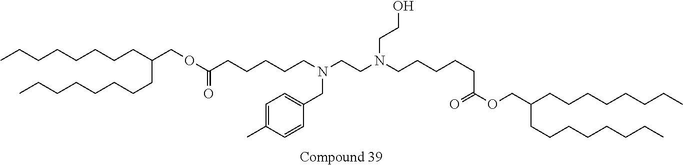 [SMILES: CCCCCCCCC(CCCCCCCC)COC(=O)CCCCCN(CCO)CCN(CCCCCC(=O)OCC(CCCCCCCC)CCCCCCCC)Cc1ccc(C)cc1] | ¹H NMR (400 MHz, CDCl₃) δ: 0.85-0.90 (m, 12H), 1.26 (s, 66H), 1.60-1.62 (m, 5H), 2.25-2.30 (m, 4H), 2.33 (s, 3H), 2.38-2.43 (m, 4H), 2.49-2.55(m, 6H), 3.49-3.53 (m, 4H), 3.95-3.97 (m, 4H), 7.11 (d, J = 8.0 Hz, 2H),7.19 (d, J = 7.6 Hz, 2H). LCMS: Rt: 2.030 min; MS m/z (ESI): 941.8 [M + H]⁺. |
| 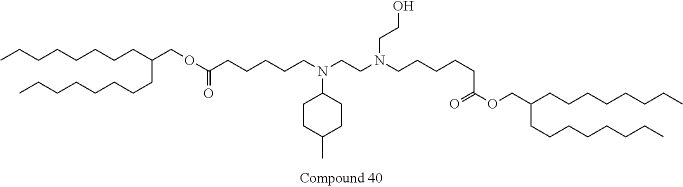 [SMILES: CCCCCCCCC(CCCCCCCC)COC(=O)CCCCCN(CCO)CCN(CCCCCC(=O)OCC(CCCCCCCC)CCCCCCCC)C1CCC(C)CC1] | ¹H NMR (400 MHz, CDCl₃) δ: 0.83-0.96 (m, 15H), 1.15-1.36 (m, 61H), 1.39-1.81 (m, 19H), 2.24- 2.35 (m, 4H), 2.36-2.63 (m, 10H), 3.47-3.58 (m, 2H), 3.91-4.03 (m, 4H). LCMS: Rt: 2.240 min; MS m/z (ESI): 933.8 [M + H]⁺. |
| 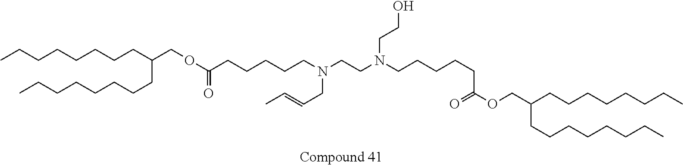 [SMILES: C/C=C/CN(CCCCCC(=O)OCC(CCCCCCCC)CCCCCCCC)CCN(CCO)CCCCCC(=O)OCC(CCCCCCCC)CCCCCCCC] | ¹H NMR (400 MHz, CDCl₃) δ: 0.82-0.94 (m, 12H), 1.15-1.36 (m, 60H), 1.39-1.54 (m, 4H),1.56-1.74 (m, 10H), 2.28-2.32 (m, 4H), 2.35- 2.65 (m, 9H), 2.97-3.11 (m, 2H), 3.45-3.56 (m, 2H), 3.91-4.03 (m, 4H), 5.44-5.68 (m, 2H). LCMS: Rt: 2.170 min; MS m/z (ESI): 891.8 [M + H]⁺. |
| 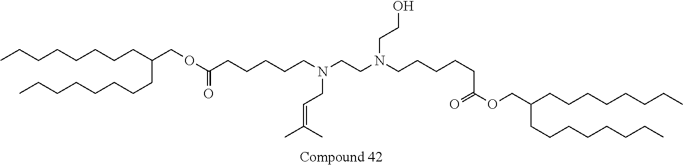 [SMILES: CCCCCCCCC(CCCCCCCC)COC(=O)CCCCCN(CC=C(C)C)CCN(CCO)CCCCCC(=O)OCC(CCCCCCCC)CCCCCCCC] | ¹H NMR (400 MHz, CDCl₃) δ: 0.81-0.93 (m, 12H), 1.67-1.36 (m, 59H), 1.39-1.80 (m, 18H), 2.22- 2.65 (m, 14H),3.02-3.14 (m, 2H), 3.46-3.58 (m, 2H), 3.90-4.03 (m, 4H), 5.18-5.29 (m, 1H). LCMS: Rt: 0.680 min; MS m/z (ESI): 905.8 [M + H]⁺. |
| 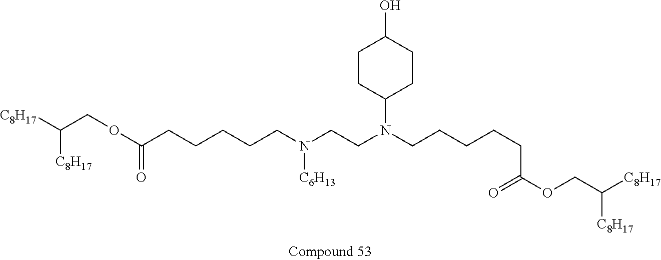 | ¹H NMR (400 MHz, CDCl₃) δ: 0.87 (t, J = 8 Hz, 15H), 1.66-1.76 (m, 85H), 1.99-2.01 (m, 2H), 2.28- 2.47 (m, 15H), 3.53-3.56 (m, 1H), 3.96-3.97 (m, 4H). LCMS: Rt: 0.627 min; MS m/z (ESI): 975.7 [M + H]⁺. |
| 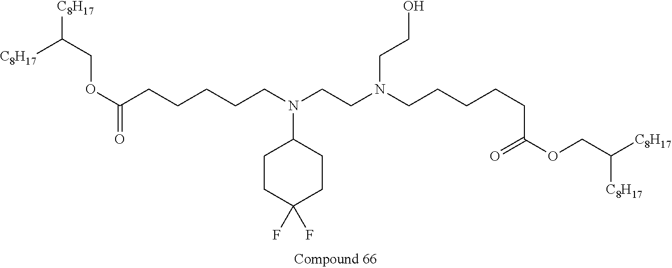 | ¹H NMR (400 MHz, CDCl₃): 0.86- 0.90 (m, 12H), 1.10-1.16 (m, 2H), 1.18-1.46 (m, 61H), 1.47-1.55 (m, 5H), 1.56-1.71 (m, 8H), 1.73-1.81 (m, 3H), 2.13-2.21 (m, 2H), 2.32- 2.38 (m, 4H), 2.39-2.56 (m, 7H), 2.57-2.67 (m, 3H), 3.47-3.57 (m, 1H), 3.87-3.99 (m, 4H). LCMS: Rt: 1.010 min; MS m/z (ESI): 955.7 [M + H]⁺. |

### 6.17 Example 17: Preparation of Compound 37

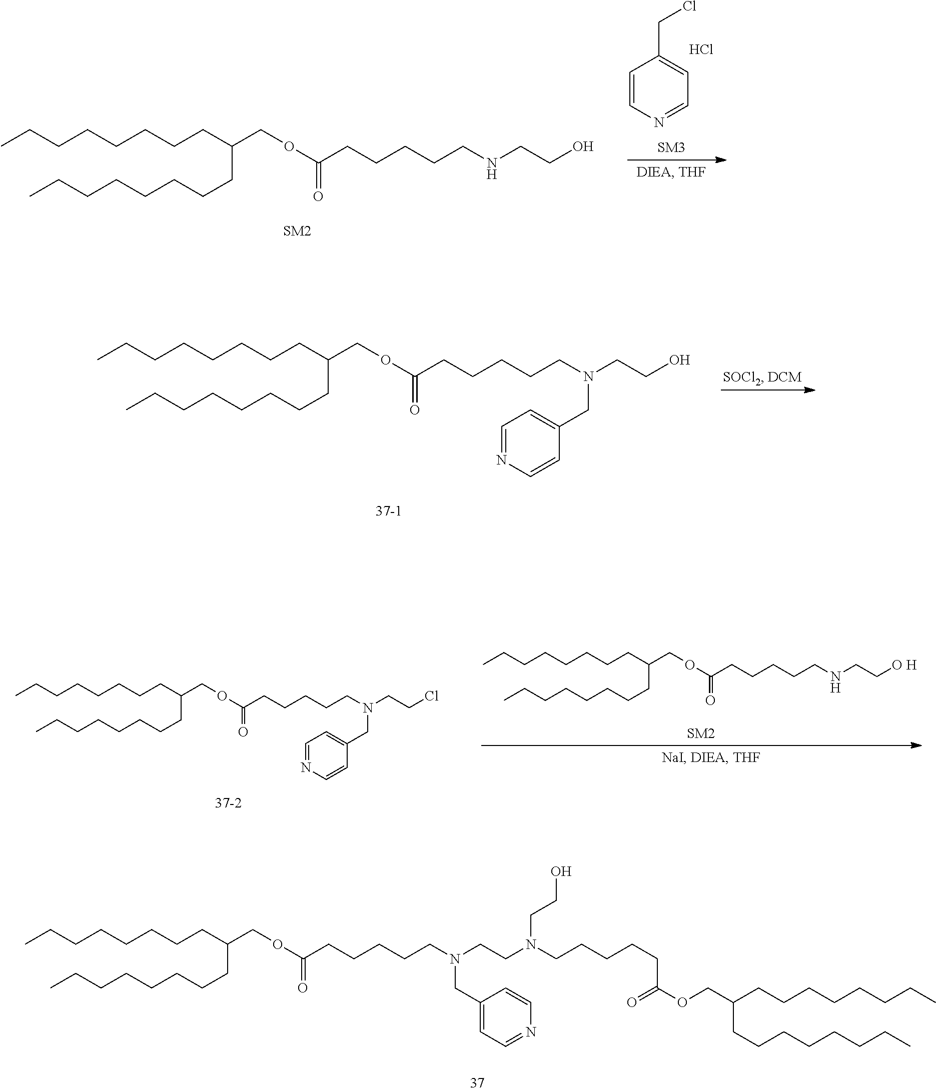

### Step 1: Preparation of Compound 37-1

A mixture of compound SM2 (200 mg, 0.47 mmol, 1.0 eq.), compound SM3 (154 mg, 0.93 mmol, 2.0 eq.), DIEA (300 mg, 2.35 mmol, 5.0 eq.) in THF (20 mL) was stirred at reflux overnight. LCMS showed the target product. The mixture was concentrated and diluted with ethyl acetate, washed with water and brine, dried over Na₂SO₄ and concentrated. The residue was purified by Pre-HPLC to give compound 37-1 (180 mg, 82% yield) as a yellow oil. LCMS: Rt: 0.940 min; MS m/z (ESI): 519.4 [M+H]⁺.

### Step 2: Preparation of Compound 37-2

A mixture of compound 37-1 (180 mg, 0.35 mmol, 1.0 eq.) and SOCl₂ (205 mg, 1.7 mmol, 5.0 eq.) in DCM (5 mL) was stirred overnight at 35° C. The mixture was concentrated to give the desired product compound 37-2 (210 mg, crude) as a yellow oil.

### Step 3: Preparation of Compound 37

A mixture of compound 37-2 (210 mg, 0.35 mmol, 1.0 eq.), compound SM2 (180 mg, 0.42 mmol, 1.2 eq.), sodium iodides (15 mg, 0.1 mmol, 0.3 eq.) and DIEA (135 mg, 1.1 mmol, 3.0 eq.) in THF (5 mL) was stirred overnight at 70° C. The mixture was concentrated under vacuum. The residual was purified by prep-HPLC to give the desired product compound 37 (43 mg, 12.4% yield) as light yellow oil. LCMS: Rt: 1.740 min; MS m/z (ESI): 928.7 [M+H]⁺.

¹H NMR (400 MHz, CDCl₃) δ: 0.86-0.90 (m, 12H), 1.26 (s, 62H), 1.38-1.43 (m, 5H), 1.61-1.64 (m, 4H), 2.26-2.30 (m, 4H), 2.41-2.44 (m, 4H), 2.51-2.57 (m, 6H), 3.50-3.52 (m, 2H), 3.57 (s, 2H), 3.96 (d, J=5.6 Hz, 4H), 7.27-7.29 (m, 2H), 8.53-8.54 (m, 2H).

### 6.18 Example 18: Preparation of Compound 43

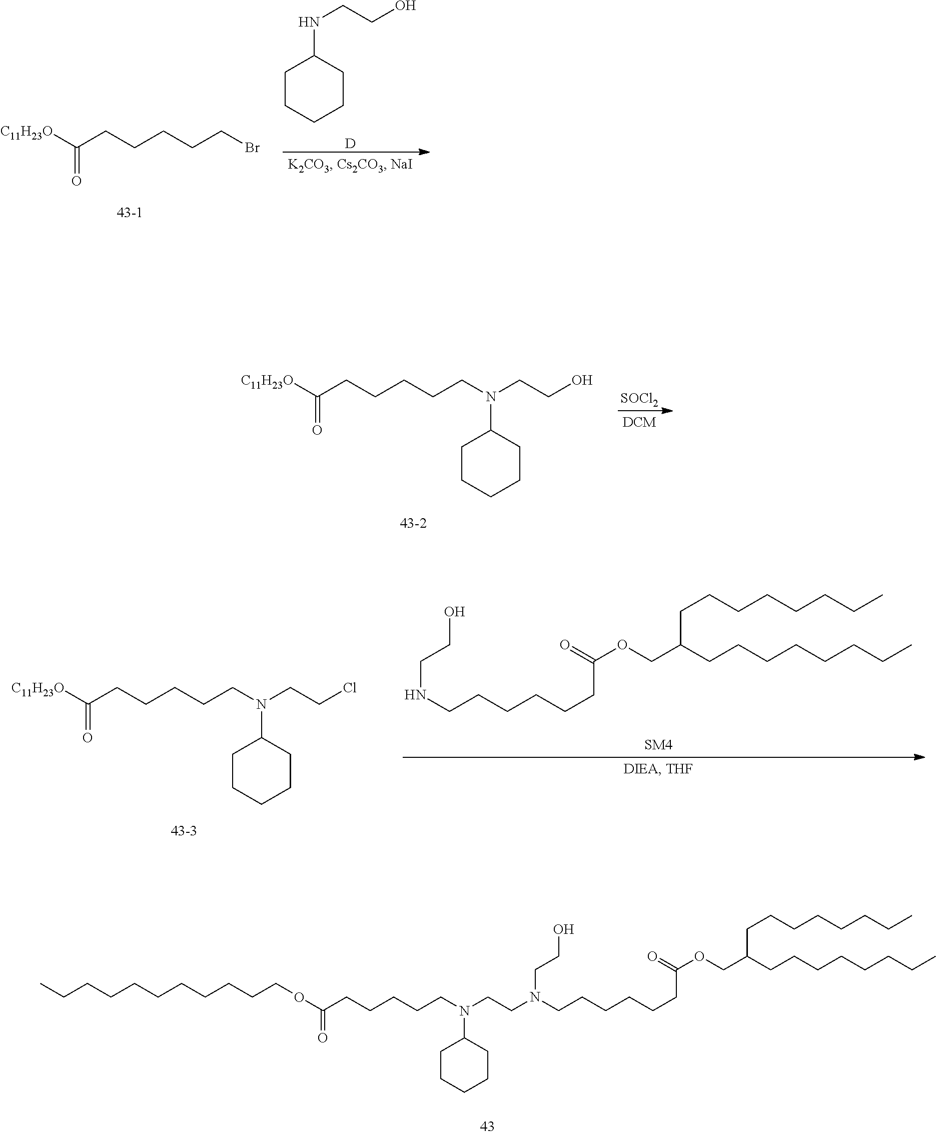

### Step 1: Preparation of Compound 43-2

A mixture of compound 43-1 (5.0 g, 14.3 mmol, 1.0 eq.), compound D (2.5 g, 17.2 mmol, 1.2 eq.), K₂CO₃ (5.9 g, 42.9 mmol, 3.0 eq.), Cs₂CO₃ (1.4 g, 4.3 mmol, 0.3 eq.), NaI (645 mg, 0.43 mmol, 0.3 eq.) in ACN (60 mL) was stirred overnight at reflux. The mixture was diluted with water, extracted with ethyl acetate, concentrated and purified by column chromatography silica gel (MeOH:DCM=0% to 10%) to give the desired product compound 43-2 (2.6 g, 44.2% yield) as a yellow oil. LCMS: Rt: 0.800 min; MS m/z (ESI): 412.3 [M+H]⁺.

### Step 2: Preparation of Compound 43-3

A mixture of compound 43-2 (400 mg, 0.97 mmol, 1.0 eq.) and SOCl₂ (580 mg, 4.9 mmol, 5.0 eq.) in DCM (10 mL) was stirred overnight at 35° C. The mixture was concentrated to give the desired product compound 43-3 (440 mg, crude) as a yellow oil.

### Step 3: Preparation of Compound 43

A mixture of compound SM4 (210 mg, 0.47 mmol, 1.0 eq.), compound 43-3 (240 mg, 0.57 mmol, 1.2 eq.), sodium iodides (21 mg, 0.1 mmol, 0.3 eq.) and DIEA (182 mg, 1.4 mmol, 3.0 eq.) in THF (5 mL) was stirred overnight at 70° C. The mixture was concentrated under vacuum. The residual was purified by prep-HPLC to give the desired product compound 43 (86 mg, 21.8% yield) as brown oil. LCMS: Rt: 1.190 min; MS m/z (ESI): 835.7 [M+H]⁺.

¹H NMR (400 MHz, CDCl₃) δ: 0.86-0.90 (m, 9H), 1.14-1.26 (m, 59H), 1.44-1.46 (m, 4H), 1.60-1.67 (m, 6H), 1.77-1.79 (m, 4H), 2.28-2.32 (m, 4H), 2.42-2.50 (m, 8H), 2.59 (s, 2H), 3.52-3.53 (m, 2H), 3.96 (d, J=5.6 Hz, 2H), 4.03-4.07 (m, 2H).

The following compounds were prepared in analogous fashion as Compound 43, using corresponding starting material.

| Compound | Characterization |
|---|---|
| 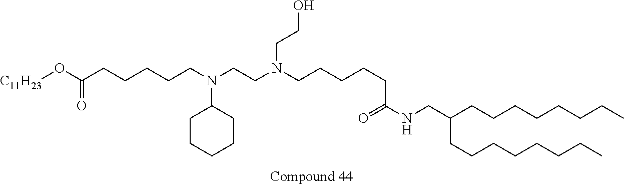 | ¹H NMR (400 MHz, CDCl₃) δ: 0.86-0.90 (m, 9H), 1.26-1.30 (m, 56H), 1.42-1.48 (m, 4H), 1.52-1.69 (m, 8H), 1.77-1.80 (m, 3H), 2.16-2.20 (m, 2H), 2.28-2.30 (m, 2H), 2.38-2.65 (m, 10H), 3.14-3.19 (m, 2H), 3.51-3.60 (m, 2H), 4.03-4.07 (m, 2H), 5.44-5.60 (m, 1H). LCMS: Rt: 1.310 min; MS m/z (ESI): 820.7 [M + H]⁺. |
| 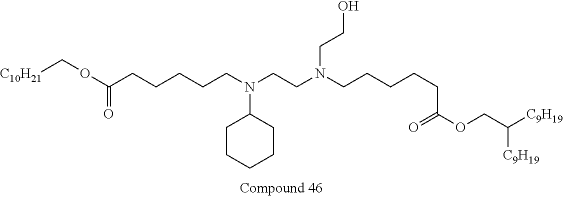 | ¹H NMR (400 MHz, CDCl₃) δ: 0.86-0.90 (m, 9H), 1.21-1.41 (m, 58H), 1.42-1.51 (m, 3H), 1.52-1.64 (m, 9H), 1.74-1.84 (m, 4H), 2.30-2.43 (m, 5H), 2.45-2.71 (m, 10H), 3.51-3.59 (m, 2H), 3.95-3.97 (m, 2H), 4.01-4.07 (m, 2H). LCMS: Rt: 1.430 min; MS m/z (ESI): 849.7 [M + H]⁺. |
| 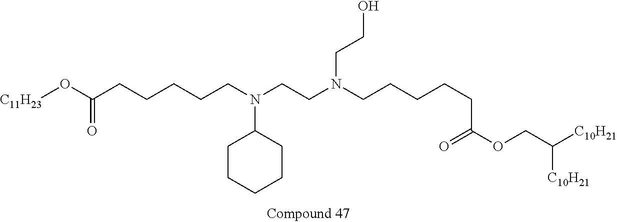 | ¹H NMR (400 MHz, CDCl₃) δ: 0.87 (t, J = 8 Hz, 9H), 1.17-1.79 (m, 78H), 2.28- 2.59 (m, 15H), 3.53-5.55 (m, 2H), 3.96- 4.07 (m, 4H). LCMS: Rt: 1.550 min; MS m/z (ESI): 877.7 [M + H]⁺. |
| 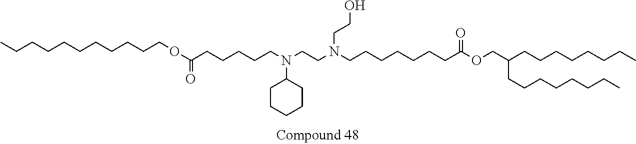 [SMILES: CCCCCCCCCCCOC(=O)CCCCCN(CCN(CCO)CCCCCCCC(=O)OCC(CCCCCCCC)CCCCCCCC)C1CCCCC1] | ¹H NMR (400 MHz, CDCl₃) δ: 0.86-0.90 (m, 9H), 1.08-1.30 (m, 61H), 1.44-1.46 (m, 4H), 1.60-1.67 (m, 6H), 1.71-1.79 (m, 4H), 2.28-2.32 (m, 4H), 2.42-2.59 (m, 10H), 3.53 (s, 2H), 3.96 (d, J = 5.6 Hz, 2H), 4.03-4.07 (m, 2H). LCMS: Rt: 1.390 min; MS m/z (ESI): 849.7 [M + H]⁺. |
| 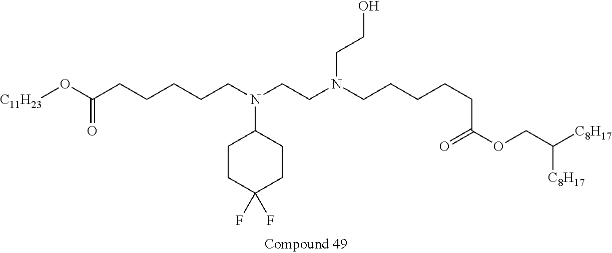 | ¹H NMR (400 MHz, CDCl₃) δ: 0.86-0.90 (m, 9H), 1.24-1.47 (m, 49H), 1.49-1.51 (m, 4H) ,1.52-1.54 (m, 1H) ,1.55-1.64 (m, 9H), 1.65-1.74 (m, 1H), 1.75-1.89 (m, 3H), 2.15-2.35 (m, 2H), 2.37-2.41 (m, 4H), 2.42-2.67 (m, 10H), 3.47-3.66 (m, 2H), 3.91-4.01 (m, 2H), 4.02-4.18 (m, 2H). LCMS: Rt: 1.300 min; MS m/z (ESI): 857.7 [M + H]⁺. |
| 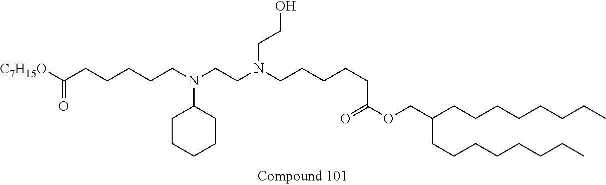 | ¹H NMR (400 MHz, CDCl₃) δ: 0.77-0.97 (m, 9H), 1.00-1.39 (m, 48H), 1.40-1.54 (m, 4H), 1.55-1.71 (m, 8H), 1.72-1.87 (m, 4H), 2.21-2.36 (m, 4H), 2.37-2.56 (m, 7H), 2.57-2.69 (m, 2H), 3.45-3.61 (m, 2H), 3.89-4.00 (m, 2H), 4.01-4.09 (m, 2H). LCMS: Rt: 0.914 min; MS m/z (ESI): 764.0 [M − H]⁻. |
| 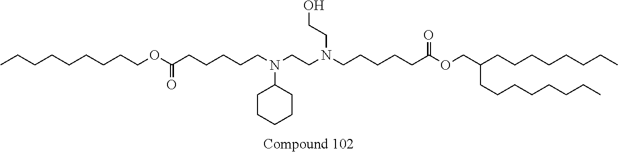 [SMILES: CCCCCCCCCOC(=O)CCCCCN(CCN(CCO)CCCCCC(=O)OCC(CCCCCCCC)CCCCCCCC)C1CCCCC1] | ¹H NMR (400 MHz, CDCl₃): 0.86-0.88 (m, 9H), 1.26 (s, 51H), 1.43-1.51 (m, 4H), 1.60-1.66 (m, 8H), 1.78 (d, J = 9.2 Hz, 4H), 2.28-2.33 (m, 4H), 2.44-2.61 (m, 10H), 3.52-3.54 (m, 2H), 3.97 (d, J = 5.6 Hz, 2H), 4.04-4.07 (m, 2H). LCMS: Rt: 1.220 min; MS m/z (ESI): 793.6 [M + H]⁺. |
| 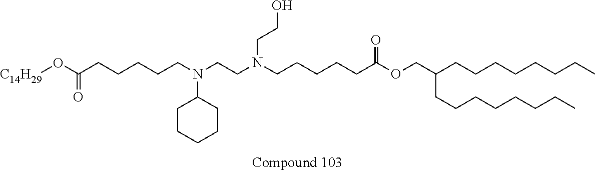 | ¹H NMR (400 MHz, CDCl₃) δ: 0.86-0.90 (m, 9H), 1.13-1.35 (m, 61H), 1.35-1.49 (m, 4H), 1.58-1.68 (m, 8H), 1.74-1.82 (m, 4H), 2.28-2.30 (m, 4H), 2.32-2.49 (m, 8H), 2.56-2.62 (m, 2H), 3.53-3.55 (m, 2H), 3.96-3.97 (m, 2H), 4.05-4.07 (m, 2H). LCMS: Rt: 1.490 min; MS m/z (ESI): 863.7 [M + H]⁺. |
| 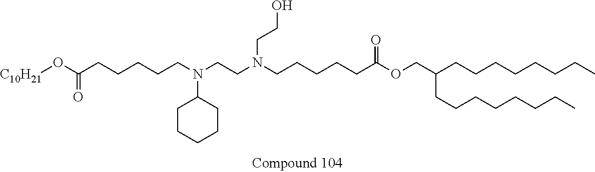 | ¹H NMR (400 MHz, CDCl₃) δ: 0.86-0.90 (m, 9H), 1.13-1.32 (m, 66H), 1.37-1.53 (m, 3H), 1.59-1.68 (m, 9H), 1.74-1.87 (m, 4H), 2.28-2.30 (m, 4H), 2.32-2.70 (m, 9H), 3.51-3.68 (m, 2H), 3.96-3.97 (m, 2H), 4.03-4.07 (m, 2H). LCMS: Rt: 1.87 min; MS m/z (ESI): 891.8 [M + H]⁺. |
| 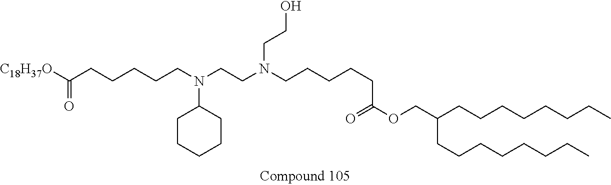 | ¹H NMR (400 MHz, CDCl₃) δ: 0.76-0.98 (m, 9H), 1.00-1.40 (m, 68H), 1.41-1.55 (m, 4H), 1.56-1.71 (m, 8H), 1.72-1.89 (m, 4H), 2.20-2.37 (m, 4H), 2.38-2.66 (m, 9H), 2.83-3.16 (m, 2H), 3.41-3.61 (m, 2H), 3.90-4.00 (m, 2H), 4.01-4.11 (m, 2H). LCMS: Rt: 2.110 min; MS m/z (ESI): 919.7 [M + H]⁺. |
| 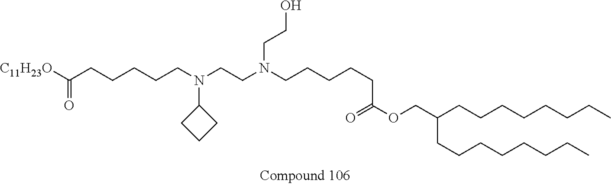 | ¹H NMR (400 MHz, CDCl₃) δ: 0.81-0.94 (m, 9H), 1.10-1.39 (m, 50H), 1.38-1.52 (m, 4H), 1.54-1.72 (m, 10H), 1.86-2.06 (m, 4H), 2.24-2.34 (m, 4H), 2.37-2.45 (m, 3H), 2.46-2.56 (m, 3H), 2.57-2.64 (m, 2H), 3.00-3.14 (m, 1H), 3.49-3.59 (m, 2H), 3.93-3.95 (m, 2H), 4.01-4.09 (m, 2H). LCMS: Rt: 2.030 min; MS m/z (ESI): 793.7 [M + H]⁺. |
| 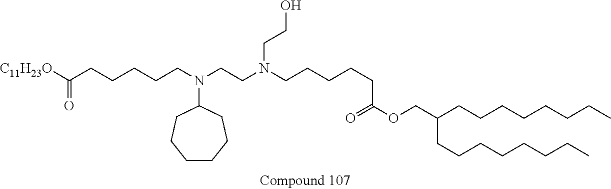 | ¹H NMR (400 MHz, CDCl₃) δ: 0.82-0.93 (m, 9H), 1.16-1.30 (m, 46H), 1.35-1.54 (m, 12H), 1.56-1.72 (m, 13H), 1.74-1.85 (m, 2H), 2.26-2.32 (m, 4H), 2.33-2.55 (m, 7H), 2.57-2.61 (m, 2H), 2.63-2.71 (m, 1H), 3.45-3.59 (m, 2H), 3.92-4.00 (m, 2H), 4.01-4.09 (m, 2H). LCMS: Rt: 1.330 min; MS m/z (ESI): 835.8 [M + H]⁺. |
| 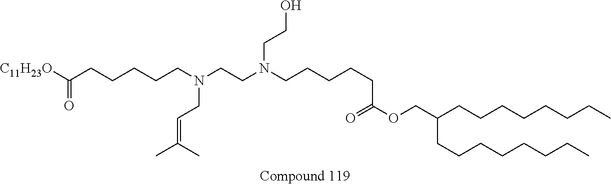 | ¹H NMR (400 MHz, CDCl₃) δ: 0.82-0.93 (m, 9H), 1.18-1.37 (m, 47H), 1.39-1.52 (m, 4H), 1.57-1.69 (m, 14H), 1.71-1.77 (m, 3H), 2.21-2.35 (m, 4H), 2.36-2.65 (m, 8H), 2.99-3.16 (m, 2H), 3.47-3.59 (m, 2H), 3.91-4.00 (m, 2H), 4.01-4.10 (m, 2H), 5.17-5.29 (m, 1H). LCMS: Rt: 1.280 min; MS m/z (ESI): 807.7 [M + H]⁺. |
| 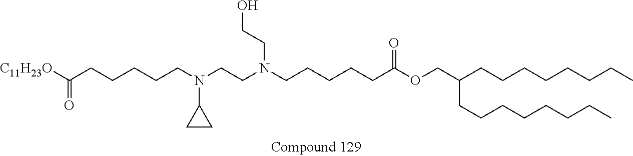 | ¹H NMR (400 MHz, CDCl₃) δ: 0.41-0.50 (m, 4H), 0.82-0.93 (m, 9H), 1.21-1.38 (m, 43H), 1.42-1.48 (m, 2H), 1.50-1.68 (m, 14H), 1.69-1.74 (m, 1H), 1.97-2.09 (m, 1H), 2.21-2.34 (m, 4H), 2.46-2.68 (m, 10H), 3.45-3.54 (m, 2H), 3.93-4.00 (m, 2H), 4.01-4.11 (m, 2H). LCMS: Rt: 1.350 min; MS m/z (ESI): 779.7 [M + H]⁺. |
| 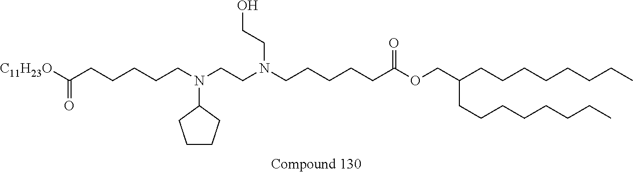 | ¹H NMR (400 MHz, CDCl₃) δ: 0.83-0.94 (m, 9H), 1.17-1.36 (m, 48H), 1.38-1.54 (m, 8H), 2.99-3.16 (m, 2H), 3.47-3.59 (m, 8H), 1.57-1.69 (m, 10H), 1.71-1.83 (m, 2H), 3.91-4.00 (m, 2H), 4.01-4.10 (m, 3H), 2.25-2.35 (m, 4H), 2.45-2.64 (m, 9H), 3.98-3.07 (m, 1H), 3.51-3.58 (m, 2H), 3.92-3.98 (m, 2H), 4.02-4.09 (m, 2H). LCMS: Rt: 1.470 min; MS m/z (ESI): 807.8 [M + H]⁺. |
| 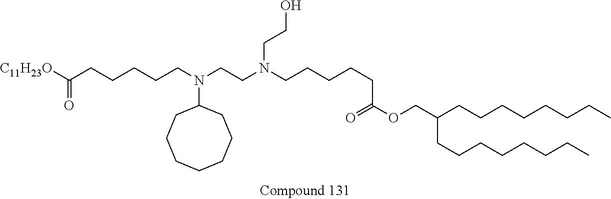 | ¹H NMR (400 MHz, CDCl₃) δ: 0.75-0.95 (m, 9H), 1.12-1.38 (m, 50H), 1.39-1.55 (m, 10H), 1.56-1.86 (m, 15H), 2.23-2.36 (m, 6H), 2.37-2.43 (m, 5H), 2.67-3.25 (m, 3H), 3.45-3.77 (m, 2H), 3.92-4.01 (m, 2H), 4.02-4.11 (m, 2H). LCMS: Rt: 1.340 min; MS m/z (ESI): 849.8 [M + H]⁺. |

### 6.19 Example 19: Preparation of Compound 50

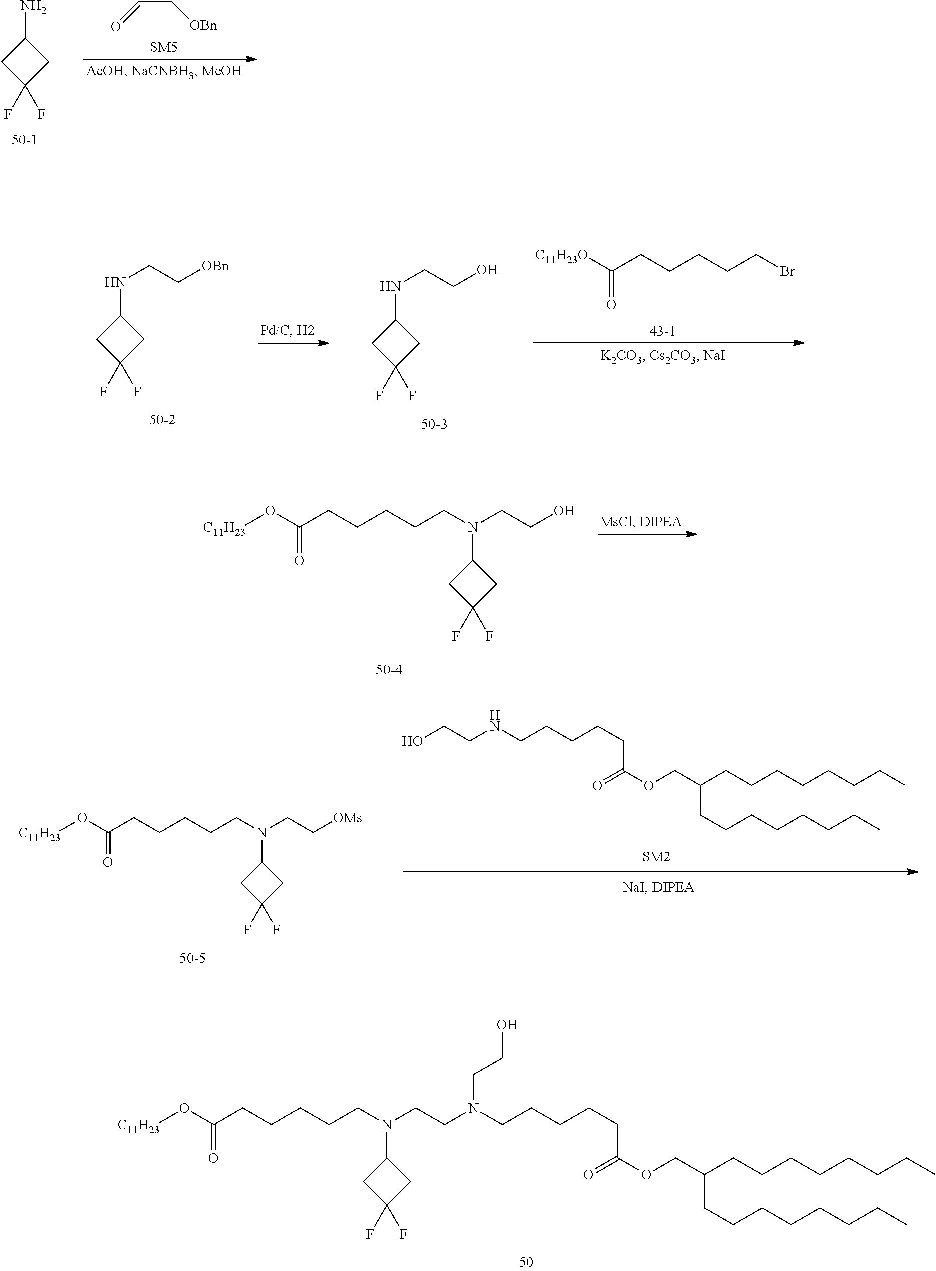

### Step 1: Preparation of Compound 50-2

To a solution of compound 50-1 (600 mg, 5.60 mmol, 1.0 eq.) in MeOH (30 mL) were added compound SM5 (841 mg, 5.60 mmol, 1.0 eq.) and AcOH (1 drop). The mixture was stirred at RT for 2 hours. Then NaCNBH₃ (387 mg, 6.16 mmol, 1.1 eq.) was added and the resulting mixture was stirred at RT for 16 hours. LCMS showed the reaction was complete. The mixture was concentrated and purified by column chromatography on silica gel (DCM/MeOH=30/1) to give the title compound (810 mg, 60% yield) as a yellow oil. LCMS: Rt: 0.737 min; MS m/z (ESI): 242.1 [M+H]⁺.

### Step 2: Preparation of Compound 50-3

To a solution of compound 50-2 (600 mg, 2.48 mmol, 1.0 eq.) in MeOH (10 mL) were added Pd/C (60 mg) and concentrated HCl (3 drops). The mixture was stirred at RT under H₂ for 16 hours. LCMS show the reaction was complete. The mixture was filtered through a pad of Celite and washed with MeOH. The filtration was concentrated to give the title compound (345 mg, 91% yield) as a yellow oil. LCMS: Rt: 0.320 min; MS m/z (ESI): 152.2 [M+H]⁺.

### Step 3: Preparation of Compound 50-4

To a solution of compound 50-3 (345 mg, 2.28 mmol, 1.0 eq.) and compound 43-1 (797 mg, 2.28 mmol, 1.0 eq.) in ACN (20 mL) were added K₂CO₃ (945 mg, 6.84 mmol, 3.0 eq.), Cs₂CO₃ (223 mg, 0.684 mmol, 0.3 eq.) and NaI (102 mg, 0.684 mmol, 0.3 eq.). The mixture was stirred at 80° C. for 16 hours. LCMS showed the reaction was complete. The reaction mixture was concentrated and purified by column chromatography on silica gel (DCM/MeOH=25/1) to give the title compound (320 mg, 33% yield) as a yellow oil. LCMS: Rt: 0.880 min; MS m/z (ESI): 420.3[M+H]⁺.

### Step 4: Preparation of Compound 50-5

To a solution of compound 50-4 (160 mg, 0.38 mmol, 1.0 eq.) and DIPEA (98 mg, 0.76 mmol, 2.0 eq.) in DCM (5 mL) was added MsCl (52 mg, 0.46 mmol, 1.2 eq.) at 0° C. The mixture was stirred at room temperature for 2 hours. LCMS show the reaction was completed. The mixture was poured into water and extracted with DCM. The combined organic layers were washed with brine, dried over Na₂SO₄ and concentrated to give the title compound (176 mg, 93% yield) as a yellow oil. It was used in the next step without further purification. LCMS: Rt: 0.800 min; MS m/z (ESI): 402.3 [M-OMs]⁺.

### Step 5: Preparation of Compound 50

To a solution of compound 50-5 (176 mg, 0.35 mmol, 1.0 eq.) and compound SM2 (150 mg, 0.35 mmol, 1.0 eq.) in THF (10 mL) were added DIPEA (226 mg, 1.75 mmol, 5.0 eq.) and NaI (16 mg, 0.11 mmol, 0.3 eq.). The mixture was stirred at 70° C. for 16 hours LCMS show the reaction was completed. The mixture was concentrated and purified by prep HPLC to give the title compound (29 mg, 10% yield) as colorless oil. LCMS: Rt: 1.430 min; MS m/z (ESI): 829.6 [M+H]⁺.

¹H NMR (400 MHz, CDCl₃) δ: 0.86-0.90 (m, 9H), 1.26-1.30 (m, 49H), 1.39-1.48 (m, 4H), 1.58-1.68 (m, 8H), 2.28-2.32 (m, 4H), 2.40-2.70 (m, 12H), 3.05-3.13 (m, 2H), 3.49-3.65 (m, 2H), 3.96-3.97 (m, 2H), 4.04-4.06 (m, 2H).

### 6.20 Example 20: Preparation of Compound 56

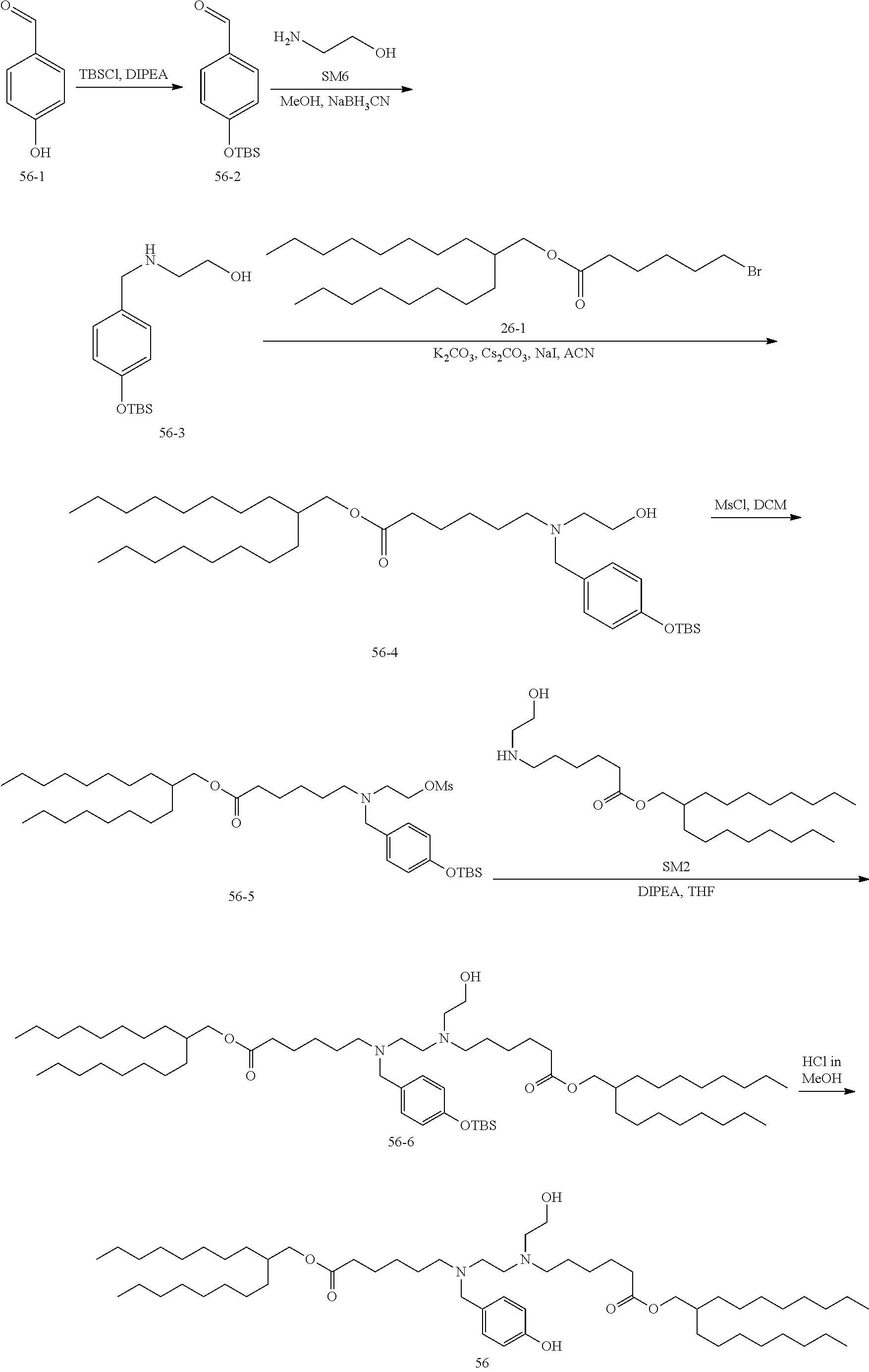

### Step 1: Preparation of Compound 56-2

To a solution of compound 56-1 (1.85 g, 15 mmol, 1.0 eq.) in DCM (30 mL) were added DIPEA (2.9 g, 22.5 mmol, 1.5 eq.) and TBSCl (2.28 g, 15 mmol, 1.0 eq.). The mixture was stirred at RT for 2 hours. The reaction mixture was poured into water and extracted with DCM. The combined organic layers were washed with brine, dried over Na₂SO₄ and concentrated. The residue was purified by column chromatography on silica gel (PE/EA=50/1) to give the title compound (1.2 g, 34% yield) as colorless oil.

¹H NMR (400 MHz, CDCl₃) δ: 0.15 (s, 6H), 0.89 (s, 9H), 6.84-6.86 (m, 2H), 7.67-7.70 (m, 2H), 9.79 (s, 1H).

### Step 2: Preparation of Compound 56-3

To a solution of compound 56-2 (1.2 g, 5.08 mmol, 1.0 eq.) in MeOH (25 mL) were added compound SM6 (465 mg, 7.62 mmol, 1.5 eq.) and AcOH (3 drops). The mixture was stirred at RT for 2 hours. Then NaCNBH₃ (383 mg, 6.10 mmol, 1.2 eq.) was added and the resulting mixture was stirred at RT for 16 hours. LCMS showed the reaction was complete. The mixture was concentrated and purified by column chromatography on silica gel (DCM/MeOH=25/1) to give the title compound (629 mg, 45% yield) as colorless oil. LCMS: Rt: 0.740 min; MS m/z (ESI): 282.2 [M+H]⁺.

### Step 3: Preparation of Compound 56-4

To a solution of compound 56-3 (629 mg, 2.23 mmol, 1.0 eq.) and compound 26-1 (1.0 g, 2.23 mmol, 1.0 eq.) in ACN (40 mL) were added K₂CO₃ (924 mg, 6.69 mmol, 3.0 eq.), Cs₂CO₃ (218 mg, 0.67 mmol, 0.3 eq.) and NaI (100 mg, 0.67 mmol, 0.3 eq.). The mixture was stirred at 80° C. for 16 hours. LCMS showed the reaction was complete. The reaction mixture was concentrated and purified by column chromatography on silica gel (DCM/MeOH=40/1) to give the title compound (290 mg, 33% yield) as colorless oil. LCMS: Rt: 1.030 min; MS m/z (ESI): 648.4 [M+H]⁺.

### Step 4: Preparation of Compound 56-5

To a solution of compound 56-4 (290 mg, 0.45 mmol, 1.0 eq.) and DIPEA (116 mg, 0.90 mmol, 2.0 eq.) in DCM (6 mL) was added MsCl (77 mg, 0.68 mmol, 1.5 eq.). The mixture was stirred at room temperature for 2 hours. LCMS showed the reaction was completed. The mixture was poured into water and extracted with DCM. The combined organic layers were washed with brine, dried over Na₂SO₄ and concentrated to give the title compound (324 mg, 100% yield) as a yellow oil. It was used in the next step without further purification. LCMS: Rt: 0.940 min; MS m/z (ESI): 630.4 [M-OMs]⁺.

### Step 5: Preparation of Compound 56-6

To a solution of compound 56-5 (324 mg, 0.45 mmol, 1.0 eq.) and compound SM2 (192 mg, 0.45 mmol, 1.0 eq.) in THF (10 mL) were added DIPEA (174 mg, 1.35 mmol, 3.0 eq.) and NaI (20 mg, 0.135 mmol, 0.3 eq.). The mixture was stirred at 70° C. for 16 hours LCMS show the reaction was completed. The mixture was concentrated and purified by column chromatography on silica gel (DCM/MeOH=30/1) to give the title compound (195 mg, 41% yield) as a yellow oil. LCMS: Rt: 0.640 min; MS m/z (ESI): 1057.7 [M+H]⁺.

### Step 6: Preparation of Compound 56

To a solution of compound 56-6 (190 mg, 0.18 mmol, 1.0 eq.) in DCM (8 mL) was added HCl in 1, 4-dioxane (2.0 mL, 4.0 M). The mixture was stirred at room temperature for 72 hours LCMS show the reaction was completed. The mixture was concentrated and purified by prep HPLC to give the title compound (32 mg, 19% yield) as a yellow oil.

¹H NMR (400 MHz, CDCl₃) δ: 0.86-0.90 (m, 12H), 1.13-1.26 (m, 65H), 1.53-1.65 (m, 8H), 2.27-2.37 (m, 6H), 2.47-2.69 (m, 7H), 3.52-3.61 (m, 4H), 3.96-4.00 (m, 4H), 6.80-6.82 (m, 2H), 7.18-7.20 (m, 2H). LCMS: Rt: 1.560 min; MS m/z (ESI): 943.5 [M+H]⁺.

### 6.21 Example 21: Preparation of Compound 57

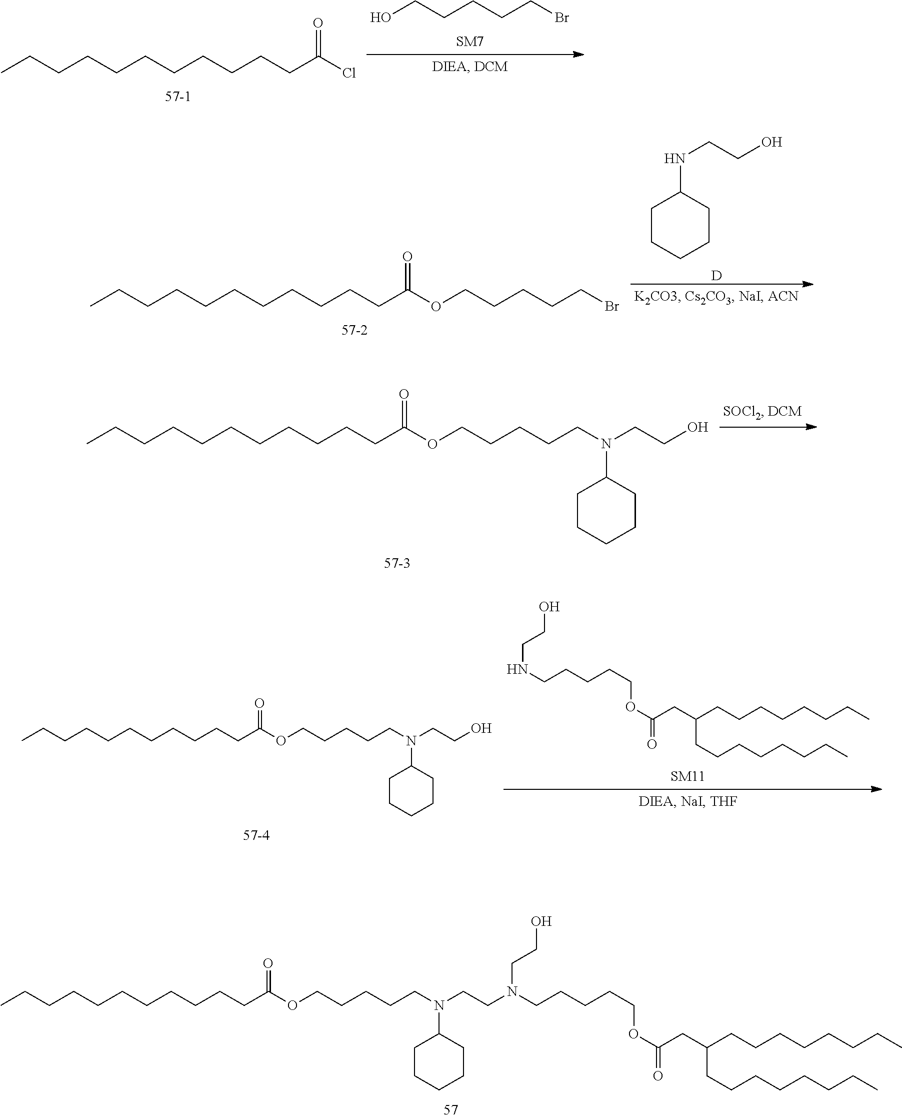

### Step 1: Preparation of Compound 57-2

A mixture of compound 57-1 (500 mg, 3.0 mmol, 1 eq.), compound SM7 (982 mg, 4.5 mmol, 1.5 eq.) and DIEA (1.16 g, 9.0 mmol, 3 eq.) in DCM (10 mL) was stirred for an hour at RT. The mixture was quenched with water, extracted over ethyl acetate, concentrated and purified by column chromatography silica gel (EA:PE=0% to 5%) to give the desired product 57-2 (942 mg, 59.0% yield) as a yellow oil.

¹H NMR (400 MHz, CDCl₃) δ: 0.83-0.93 (m, 3H), 1.14-1.38 (m, 16H), 1.45-1.55 (m, 2H), 1.56-1.73 (m, 4H), 1.82-1.95 (m, 2H), 2.25-2.34 (m, 2H), 3.37-3.48 (m, 2H), 4.02-4.12 (m, 2H).

### Step 2: Preparation of Compound 57-3

A mixture of compound 57-2 (600 mg, 1.68 mmol, 1.0 eq.), compound D (291 mg, 2.01 mmol, 1.2 eq.), K₂CO₃ (696 mg, 5.04 mmol, 3.0 eq.), Cs₂CO₃ (21 mg, 0.05 mmol, 0.03 eq.) and sodium iodide (90 mg, 0.6 mmol, 0.4 eq.) in ACN (24 mL) was stirred overnight at 100° C. The mixture was concentrated and purified by column chromatography silica gel (MeOH:DCM=0% to 3%) to give the desired product 57-3 (344 mg, 29.1% yield) as a yellow oil.

¹H NMR (400 MHz, CDCl₃) δ: 0.83-0.93 (m, 3H), 1.14-1.44 (m, 26H), 1.56-1.73 (m, 9H), 2.25-2.33 (m, 2H), 2.56-2.81 (m, 4H), 3.36-3.45 (m, 2H), 4.03-4.11 (m, 2H).

### Step 3: Preparation of Compound 57-4

A mixture of compound 57-3 (344 mg, 0.84 mmol, 1.0 eq.) and SOCl₂ (298 mg, 2.51 mmol, 3.0 eq.) in DCM (10 mL) was stirred overnight at 35° C. The mixture was concentrated to give the desired product 57-4 (364 mg, crude) as a yellow oil. LCMS: Rt: 0.840 min; MS m/z (ESI): 430.3 [M+H]⁺.

### Step 4: Preparation of Compound 57

A mixture of compound 57-4 (172 mg, 0.4 mmol, 1.0 eq.), compound SM11 (150 mg, 0.35 mmol, 0.9 eq.), sodium iodide (30 mg, 0.2 mmol, 0.5 eq.) and DIEA (155 mg, 1.2 mmol, 3.0 eq.) in THF (5 mL) was stirred overnight at 70° C. The mixture was concentrated under vacuum. The residual was purified by prep-HPLC to give the desired product 57 (68 mg, 20.7% yield) as a yellow oil.

¹H NMR (400 MHz, CDCl₃) δ: 0.80-0.94 (m, 9H), 1.14-1.39 (m, 53H), 1.41-1.53 (m, 5H), 1.56-1.69 (m, 8H), 1.73-1.84 (m, 5H), 2.18-2.32 (m, 4H), 2.38-2.54 (m, 8H), 2.56-2.62 (m, 2H), 3.48-3.59 (m, 2H), 3.48-3.59 (m, 4H). LCMS: Rt: 1.280 min; MS m/z (ESI): 821.6 [M+H]⁺.

The following compounds were prepared in analogous fashion as Compound 57, using corresponding starting material.

| Compound | Characterization |
|---|---|
| 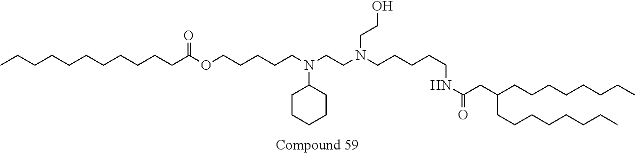 [SMILES: CCCCCCCCCCCC(=O)OCCCCCN(CCN(CCO)CCCCCNC(=O)CC(CCCCCCCC)CCCCCCCC)C1CCCCC1] | ¹H NMR (400 MHz, CDCl₃) δ: 0.76-0.95 (m, 9H), 1.04-1.36 (m, 47H), 1.37-1.90 (m, 31H), 2.00-2.10 (m, 2H), 2.23-2.33 (m, 2H), 2.36-2.67 (m, 6H), 3.15-3.29 (m, 1H), 3.45-3.60 (m, 1H), 3.98-4.11 (m, 1H). LCMS: Rt: 1.160 min; MS m/z (ESI): 820.7 [M + H]⁺. |
| 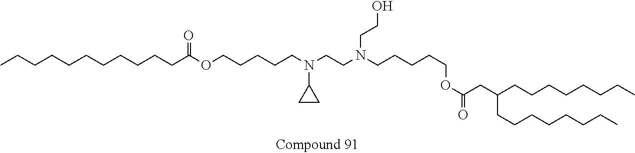 [SMILES: CCCCCCCCCCCC(=O)OCCCCCN(CCN(CCO)CCCCCOC(=O)CC(CCCCCCCC)CCCCCCCC)C1CC1] | ¹H NMR (400 MHz, CDCl₃) δ: 0.82-0.93 (m, 9H), 1.16-1.38 (m, 46H), 1.42-1.71 (m, 21H), 2.18-2.33 (m, 4H), 2.43-2.70 (m, 8H), 3.46-3.53 (m, 2H), 4.01-4.12 (m, 4H). LCMS: Rt: 1.330 min; MS m/z (ESI): 779.7 [M + H]⁺. |
| 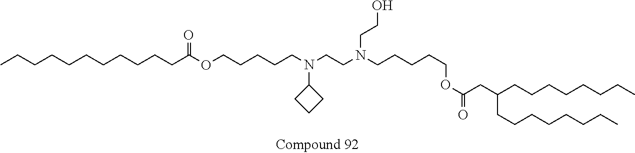 [SMILES: CCCCCCCCCCCC(=O)OCCCCCN(CCN(CCO)CCCCCOC(=O)CC(CCCCCCCC)CCCCCCCC)C1CCC1] | 1H NMR (400 MHz, CDCl₃) δ: 0.71-0.94 (m, 9H), 1.09-1.40 (m, 50H), 1.41-1.56 (m, 3H), 1.57-1.74 (m, 8H), 1.75-1.90 (m, 2H), 1.91-2.13 (m, 3H), 2.14-2.25 (m, 2H), 2.26-2.31 (m, 2H), 2.32-2.81 (m, 8H), 2.82-3.13 (m, 3H), 3.30-3.78 (m, 2H), 4.05-4.06 (m, 4H). LCMS: Rt: 1.150 min; MS m/z (ESI): 793.5 [M + H]⁺. |
| 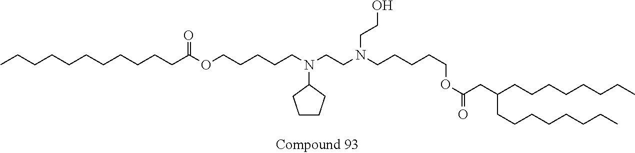 [SMILES: CCCCCCCCCCCC(=O)OCCCCCN(CCN(CCO)CCCCCOC(=O)CC(CCCCCCCC)CCCCCCCC)C1CCCC1] | ¹H NMR (400 MHz, CDCl₃) δ: 0.78-0.94 (m, 9H), 1.13-1.38 (m, 46H), 1.40-1.55 (m, 8H), 1.56-1.70 (m, 10H), 1.72-1.87 (m, 6H), 2.19-2.23 (m, 2H), 2.26-2.33 (m, 2H), 2.47-2.63 (m, 8H), 2.95-3.11 (m, 1H), 3.49-3.59 (m, 2H), 3.99- 4.11 (m, 4H). LCMS: Rt: 1.300 min; MS m/z (ESI): 807.6 [M + H]⁺. |
| 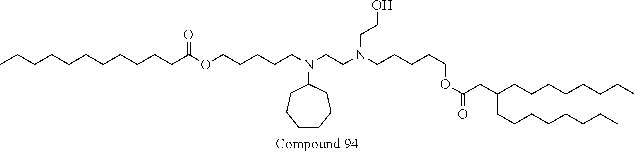 [SMILES: CCCCCCCCCCCC(=O)OCCCCCN(CCN(CCO)CCCCCOC(=O)CC(CCCCCCCC)CCCCCCCC)C1CCCCCC1] | ¹H NMR (400 MHz, CDCl₃) δ: 0.83-0.92 (m, 9H), 1.20-1.41 (m, 50H), 1.43-1.54 (m, 7H), 1.56-1.72 (m, 12H), 1.74-1.91 (m, 4H), 2.19-2.24 (m, 2H), 2.25-2.32 (m, 2H), 2.34-2.45 (m, 4H), 2.46-2.55 (m, 3H), 2.56-2.62 (m, 2H), 2.64-2.72 (m, 1H), 3.48-3.57 (m, 2H), 4.00-4.10 (m, 4H). LCMS: Rt: 1.300 min; MS m/z (ESI): 835.6 [M + H]⁺. |
| 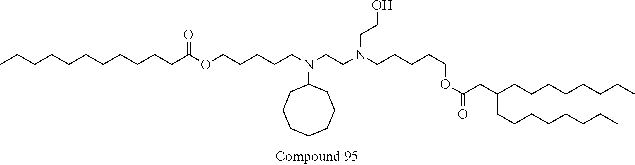 [SMILES: CCCCCCCCCCCC(=O)OCCCCCN(CCN(CCO)CCCCCOC(=O)CC(CCCCCCCC)CCCCCCCC)C1CCCCCCC1] | ¹H NMR (400 MHz, CDCl₃): 0.86-0.89 (m, 9H), 1.26 (s, 51H), 1.43-1.48 (m, 10H), 1.62-1.73 (m, 12H), 1.83 (s, 1H), 2.21-2.59 (m, 14H), 2.82 (s, 1H), 3.53 (s, 2H), 4.04-4.07 (m, 4H). LCMS: Rt: 1.520 min; MS m/z (ESI): 849.7 [M + H]⁺. |

### 6.22 Example 22: Preparation of Compound 58

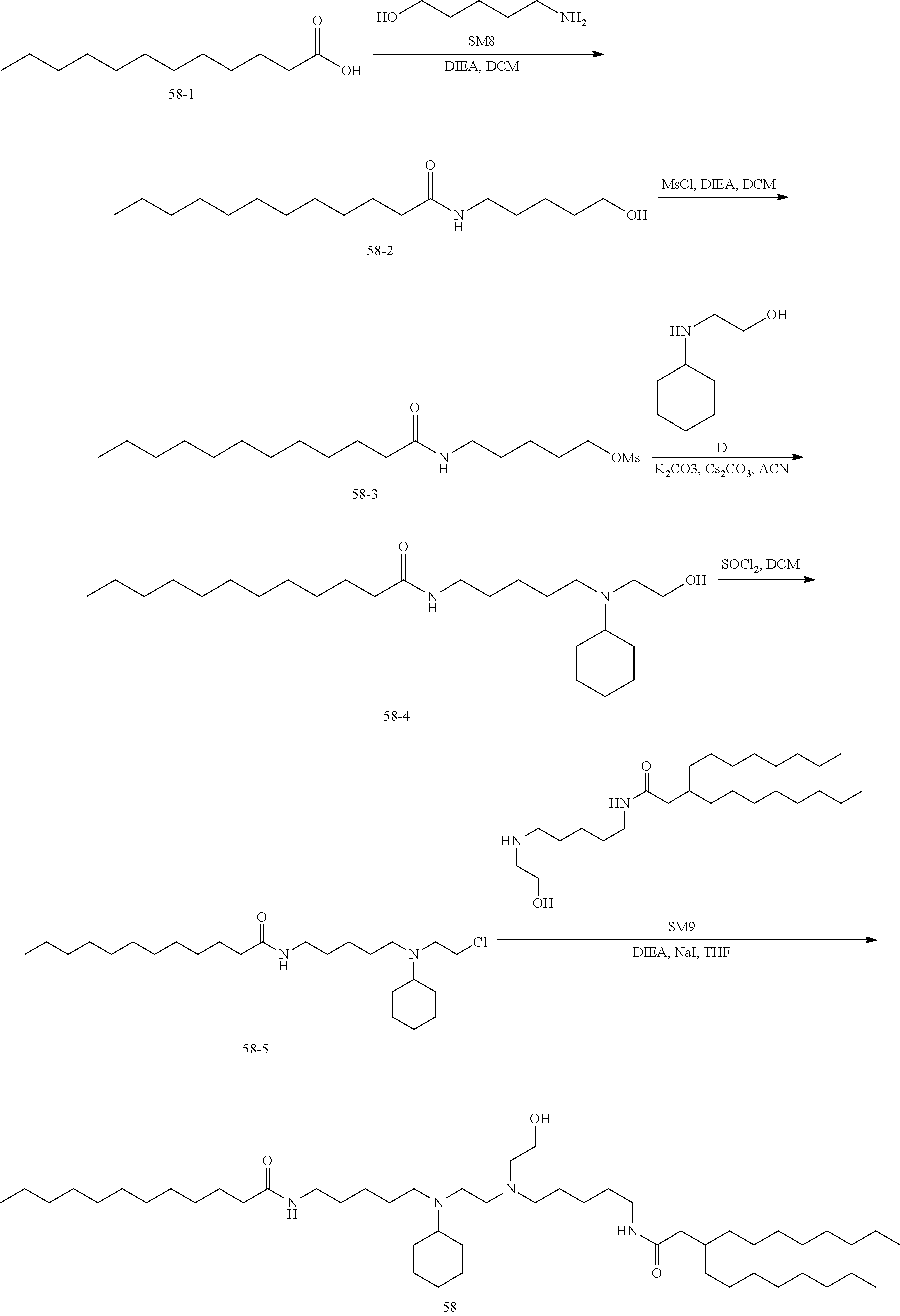

### Step 1: Preparation of Compound 58-2

A mixture of compound 58-1 (500 mg, 2.5 mmol, 1.0 eq.), compound SM8 (283 mg, 2.8 mmol, 1.51 eq.), HATU (1.1 g, 2.8 mmol, 1.1 eq.) and DIEA (483 mg, 3.8 mmol, 1.5 eq.) in DCM (10 mL) was stirred for an hour at RT. The mixture was quenched with water, extracted over ethyl acetate, concentrated and purified by column chromatography silica gel (EA:PE=0% to 67%) to give the desired product 58-2 (693 mg, 97.2% yield) as white solid. LCMS: Rt: 1.410 min; MS m/z (ESI): 286.3 [M+H]⁺.

### Step 2: Preparation of Compound 58-3

A mixture of compound 58-2 (300 mg, 1.05 mmol, 1.0 eq.), MsCl (144 mg, 1.26 mmol, 1.2 eq.) and DIEA (204 mg, 1.58 mmol, 1.5 eq.) in DCM (10 mL) was stirred for an hour at RT. The mixture was quenched with water, extracted over ethyl acetate, concentrated to give the desired product 58-3 (382 mg, crude) as a yellow solid. LCMS: Rt: 1.110 min; MS m/z (ESI): 364.2 [M+H]⁺.

### Step 3: Preparation of Compound 58-4

A mixture of compound 58-3 (382 mg, 1.05 mmol, 1.0 eq.), compound D (226 mg, 1.58 mmol, 1.5 eq.), K₂CO₃ (435 mg, 3.15 mmol, 3.0 eq.), Cs₂CO₃ (10 mg, 0.03 mmol, 0.03 eq.) in ACN (10 mL) was stirred overnight at 100° C. The mixture was concentrated and purified by column chromatography silica gel (MeOH:DCM=0% to 3%) to give the desired product 58-4 (162 mg, 37.5% yield) as a yellow oil. LCMS: Rt: 0.750 min; MS m/z (ESI): 411.3 [M+H]⁺.

### Step 4: Preparation of Compound 58-5

A mixture of compound 58-4 (162 mg, 0.39 mmol, 1.0 eq.) and SOCl₂ (140 mg, 1.18 mmol, 3.0 eq.) in DCM (10 mL) was stirred overnight at 35° C. The mixture was concentrated to give the desired product 58-5 (187 mg, crude) as a yellow oil. LCMS: Rt: 0.780 min; MS m/z (ESI): 429.3 [M+H]⁺.

### Step 5: Preparation of Compound 58

A mixture of compound 58-5 (187 mg, 0.4 mmol, 1.0 eq.), compound SM9 (100 mg, 0.2 mmol, 0.5 eq.), sodium iodide (30 mg, 0.2 mmol, 0.5 eq.) and DIEA (155 mg, 1.2 mmol, 3.0 eq.) in THF (5 mL) was stirred overnight at 70° C. The mixture was concentrated under vacuum. The residual was purified by prep-HPLC to give the desired product 58 (22 mg, 6.2% yield) as a yellow oil. LCMS: Rt: 1.000 min; MS m/z (ESI): 819.6 [M+H]⁺.

¹H NMR (400 MHz, CDCl₃) δ: 0.79-0.94 (m, 9H), 1.08-1.37 (m, 53H), 1.42-1.55 (m, 8H), 1.57-1.65 (m, 5H), 1.75-1.91 (m, 8H), 2.03-2.10 (m, 2H), 2.11-2.20 (m, 2H), 2.43-2.64 (m, 9H), 3.17-3.28 (m, 4H), 3.48-3.62 (m, 2H).

The following compounds were prepared in analogous fashion as Compound 58, using corresponding starting material.

| Compound | Characterization |
|---|---|
| 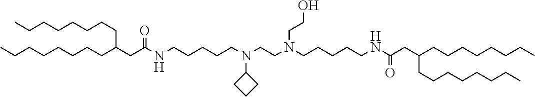 [SMILES: CCCCCCCCC(CCCCCCCC)CC(=O)NCCCCCN(CCO)CCN(CCCCCNC(=O)CC(CCCCCCCC)CCCCCCCC)C1CCC1] Compound 60 | ¹H NMR (400 MHz, CDCl₃) δ: 0.86-0.90 (m, 12H), 1.25 (s, 56H), 1.43-1.68 (m, 12H), 1.86-2.08 (m, 12H), 2.19- 2.25 (m, 1H), 2.44-2.59 (m, 9H), 3.10 (s, 1H), 3.21-3.26 (m, 4H), 3.54-3.56 (m, 2H), 5.35 (s, 1H), 5.74 (s, 1H), |
| | 5.89 (s, 1H). |
| | LCMS: Rt: 1.430 min; |
| | MS m/z (ESI): 889.7 [M + H]⁺. |
| 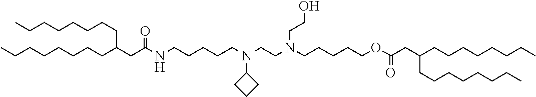 [SMILES: CCCCCCCCC(CCCCCCCC)CC(=O)NCCCCCN(CCN(CCO)CCCCCOC(=O)CC(CCCCCCCC)CCCCCCCC)C1CCC1] Compound 61 | ¹H NMR (400 MHz, CDCl₃): 0.86-0.90 (m, 12H), 1.26 (s, 60H), 1.43-1.66 (m, 10H), 1.84-2.07 (m, 9H), 2.22 (d, J = 6.8 Hz, 2H), 2.42-2.60 (m, 10H), 3.03-3.10 (m, 1H), 3.21-3.26 (m, 2H), 3.54-3.57 (m, 2H), 4.03-4.07 (m, 2H), |
| | 5.70 (s, 1H). |
| | LCMS: Rt: 1.520 min; |
| | MS m/z (ESI): 890.7 [M + H]⁺. |

### 6.23 Example 23: Preparation of Compound 62

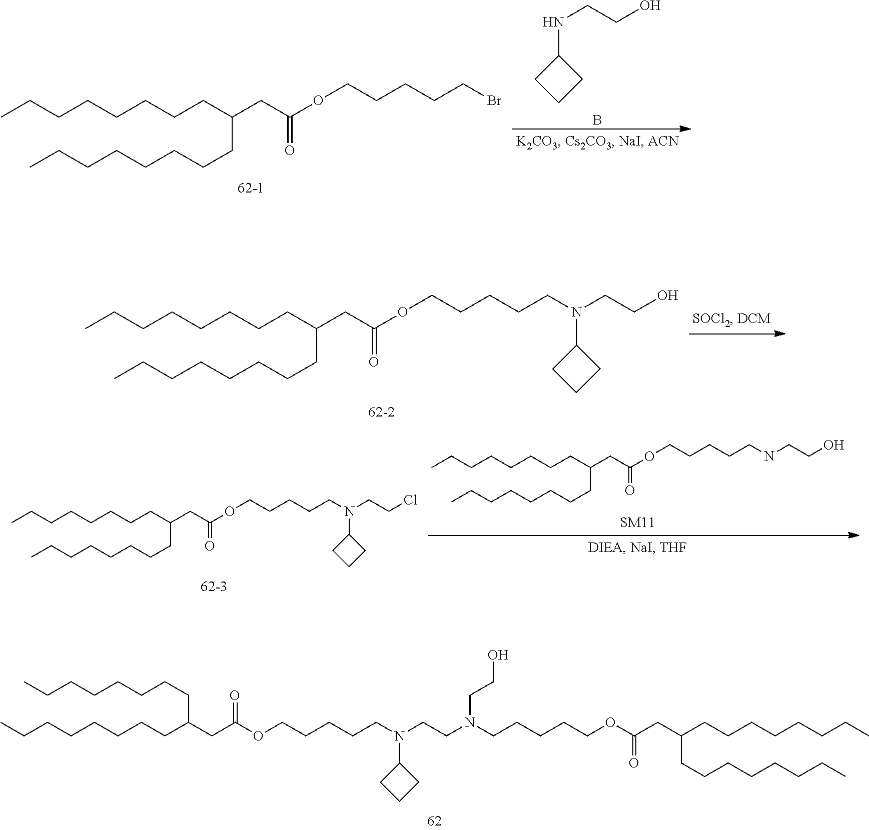

### Step 1: Preparation of Compound 62-2

To a solution of compound 62-1 (0.4 g, 0.8939 mmol, 1.0 eq.) in ACN (15 mL) was added compound B (124 mg, 1.073 mmol, 1.2 eq.), K₂CO₃ (371 mg, 2.682 mmol, 3.0 eq.), Cs₂CO₃ (87 mg, 0.2682 mmol, 0.3 eq.), NaI (13 mg, 0.08939 mmol, 0.1 eq.). The reaction mixture was stirred at 80° C. for 16 hours. LCMS showed the reaction was complete. Removal of solvent, FCC to get the compound 62-2 (330 mg, 79.28%) as a yellow oil. LCMS: Rt: 0.970 min; MS m/z (ESI): 482.4 [M+H]⁺.

### Step 2: Preparation of Compound 62-3

To a solution of compound 62-2 (200 mg, 0.4151 mmol, 1.0 eq.) in DCM (15 mL) was added SOCl₂ (148 mg, 1.245 mmol, 3.0 eq.). The reaction mixture was stirred at 35° C. for 16 hours. LCMS showed the reaction was complete. Removal of solvent to get the compound 62-3 (200 mg, crude) as a yellow oil. LCMS: Rt: 1.140 min; MS m/z (ESI): 500.4 [M+H]⁺.

### Step 3: Preparation of Compound 62

To a mixture of compound 62-3 (200 mg, 0.3998 mmol, 1.14 eq.), DIEA (136 mg, 1.052 mmol, 3.0 eq.) in THF (15 mL) was added compound SM11 (150 mg, 0.3507 mmol, 1.0 eq.), NaI (15 mg). The reaction mixture was stirred at 75° C. for 64 hours. LCMS showed the reaction was complete. After removal of solvent, the residue was purified by pre-HPLC to give the title compound (80 mg, 25.59% yield) as a yellow oil. LCMS: Rt: 1.790 min; MS m/z (ESI): 891.7 [M+H]⁺.

¹H NMR (400 MHz, CDCl₃): 0.86-0.90 (m, 12H), 1.26 (s, 59H), 1.44-1.65 (m, 11H), 1.83-2.00 (m, 7H), 2.22 (d, J=6.8 Hz, 4H), 2.42-2.62 (m, 10H), 3.08 (s, 1H), 3.54-3.56 (m, 2H), 4.03-4.07 (m, 4H).

### 6.24 Example 24: Preparation of Compound 64

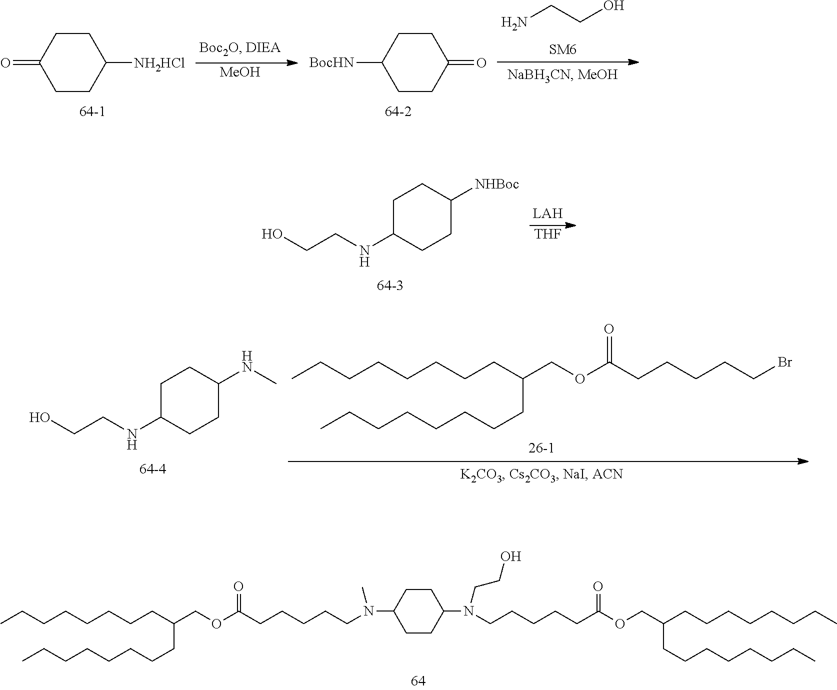

### Step 1: Preparation of Compound 64-2

To a mixture of compound 64-1 (2.0 g, 13.4 mmol, 1.0 eq.), DIEA (4.2 g, 32.1 mmol, 2.4 eq.) in DCM (30 mL) was added Boc₂O (3.5 g, 16.0 mmol, 1.2 eq.). The mixture was stirred at RT for 2 hours, TLC showed the reaction was complete. The mixture was diluted with DCM and washed with water and brine, dried, concentrated and the residue was purified by column chromatography to give product 64-2 (2.3 g, 80% yield) as white solid.

¹H NMR (400 MHz, CDCl₃) δ: 1.46 (s, 9H), 1.62-1.72 (m, 2H), 2.22-2.26 (m, 2H), 2.38-2.46 (m, 4H), 3.93 (s, 1H), 4.50 (s, 1H).

### Step 2: Preparation of Compound 64-3

A mixture of compound 64-2 (2.3 g, 10.8 mmol, 1 eq.), compound SM6 (2.0 g, 32.3 mmol, 3.0 eq.), NaBH₃CN (1.4 g, 21.7 mmol, 2.0 eq.) in MeOH (30 mL) was stirred at reflux overnight. LCMS showed the reaction was complete. The mixture was diluted with ethyl acetate and washed with water and brine, dried, concentrated and the residue was purified by column chromatography to give product 64-3 (1.6 g, 57% yield) as a yellow oil.

### Step 3: Preparation of Compound 64-4

To a solution of compound 64-3 (1.6 g, 6.2 mmol, 1.0 eq.) in THF (30 mL) was added LAH (470 mg, 12.4 mmol, 2.0 eq.) at RT. The mixture was stirred at reflux for 2 hours, LCMS showed the target product. The mixture was quenched by water and filtered, concentrated. The residue was used for the next step without further purification. LCMS: Rt: 0.290 min; MS m/z (ESI): 173.2 [M+H]⁺.

### Step 4: Preparation of Compound 64

A mixture of compound 64-4 (60 mg, 0.35 mmol, 1.0 eq.), compound 26-1 (390 mg, 0.87 mmol, 2.5 eq.), K₂CO₃ (144 mg, 1.04 mmol, 3.0 eq.), Cs₂CO₃ (33 mg, 0.1 mmol, 0.3 eq.), NaI (15 mg, 0.1 mmol, 0.3 eq.) in ACN (10 mL) was stirred at reflux overnight. LCMS showed the reaction was complete. The mixture was diluted with ethyl acetate and washed with water and brine, dried, concentrated. The residue was purified by Pre-HPLC to give product 64 (14 mg, 4.4% yield) as a yellow oil. LCMS: Rt: 1.340 min; MS m/z (ESI): 905.8 [M+H]⁺.

¹H NMR (400 MHz, CDCl₃) δ: 0.86-0.90 (m, 12H), 1.26 (s, 58H), 1.42-1.47 (m, 6H), 1.61-1.68 (m, 10H), 1.78-1.86 (m, 5H), 2.23 (s, 3H), 2.29-2.32 (m, 5H), 2.37-2.46 (m, 5H), 2.57-2.60 (m, 2H), 3.45-3.48 (m, 2H), 3.97 (d, J=6.0 Hz, 4H).

### 6.25 Example 25: Preparation of Compound 65

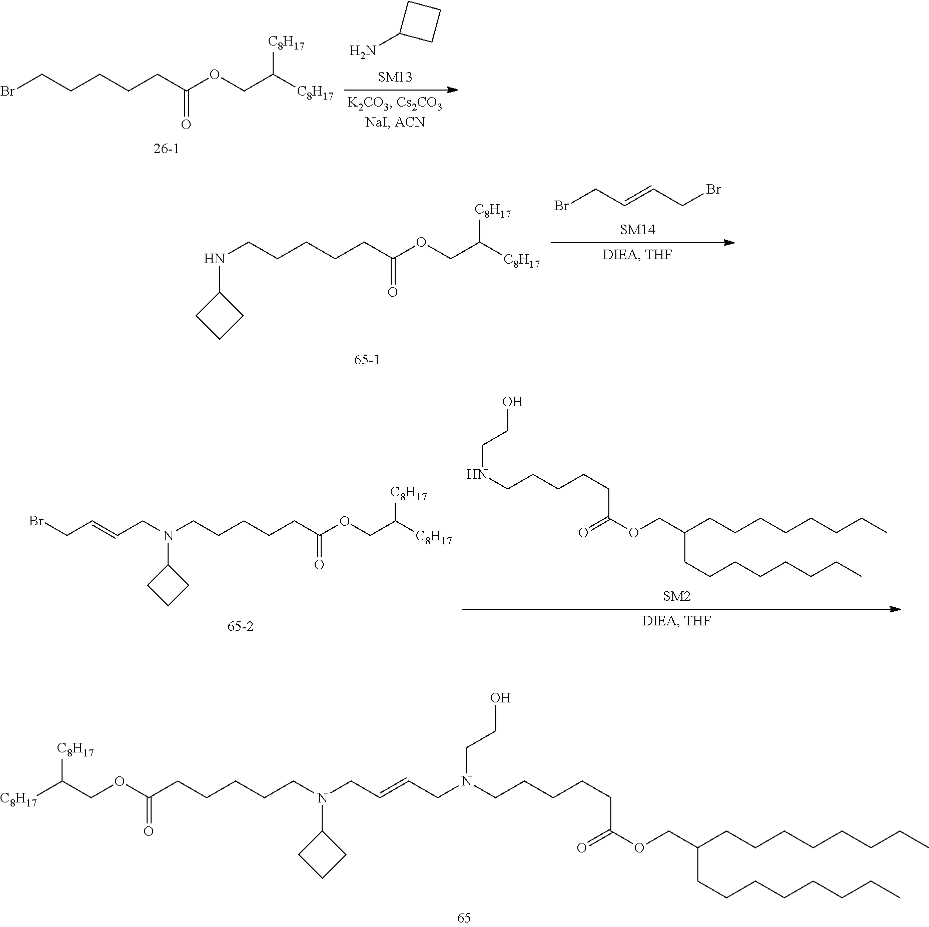

### Step 1: Preparation of Compound 65-1

To a solution of compound 26-1 (892.0 mg, 2.0 mmol, 1.0 eq.) and compound SM13 (426.0 mg, 6.0 mmol, 3.0 eq.) was dissolved in ACN (10.0 mL) was added Cs₂CO₃ (195.0 mg, 0.6 mmol, 0.3 eq.), K₂CO₃ (826.0 mg, 6.0 mmol, 3.0 eq.) and NaI (29 mg, 0.2 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide 65-1 (0.6 g, 68% yield) as a yellow oil. LCMS: Rt: 0.950 min; MS m/z (ESI): 438.3 [M+H]⁺.

### Step 2: Preparation of Compound 65-2

To a solution of compound 65-1 (100.0 mg, 0.23 mmol, 1.0 eq.) and compound SM14 (58.0 mg, 0.27 mmol, 1.2 eq.) in THF (5.0 mL) was added DIEA (44.0 mg, 0.34 mmol, 1.5 eq.) at RT. The mixture was stirred for 16 hours at 50° C. LCMS show the reaction was completed, the mixture was evaporated to provide 65-2 (230.0 mg, crude) as brown oil. LCMS: Rt: 0.443 min; MS m/z (ESI): 572.2 [M+H]⁺.

### Step 3: Preparation of Compound 65

To a solution of compound 65-2 (230.0 mg, 0.4 mmol, 1.0 eq.) and compound SM2 (207.0 mg, 0.48 mmol, 1.2 eq.) in THF (5.0 mL) was added DIEA (258 mg, 2.0 mmol, 5.0 eq.) at 0° C. The mixture was stirred for 16 hours at 70° C. LCMS show the reaction was completed, the mixture was evaporated under reduced pressured purified with pre-HPLC to provide 65 (32.0 mg, 9% yield) as a yellow oil.

¹H NMR (400 MHz, CDCl3) δ: 0.86-0.90 (m, 12H), 1.13-1.38 (m, 64H), 1.41-1.70 (m, 13H), 2.02-2.04 (m, 2H), 2.28-2.39 (m, 4H), 2.41-2.60 (m, 5H), 3.09-3.13 (m, 4H), 3.54-3.55 (m, 2H), 3.96 (d, J=0.4 Hz, 4H), 5.62-5.66 (m, 2H). LCMS: Rt: 0.581 min; MS m/z (ESI): 917.6 [M+H]⁺.

### 6.26 Example 26: Preparation of Compound 67

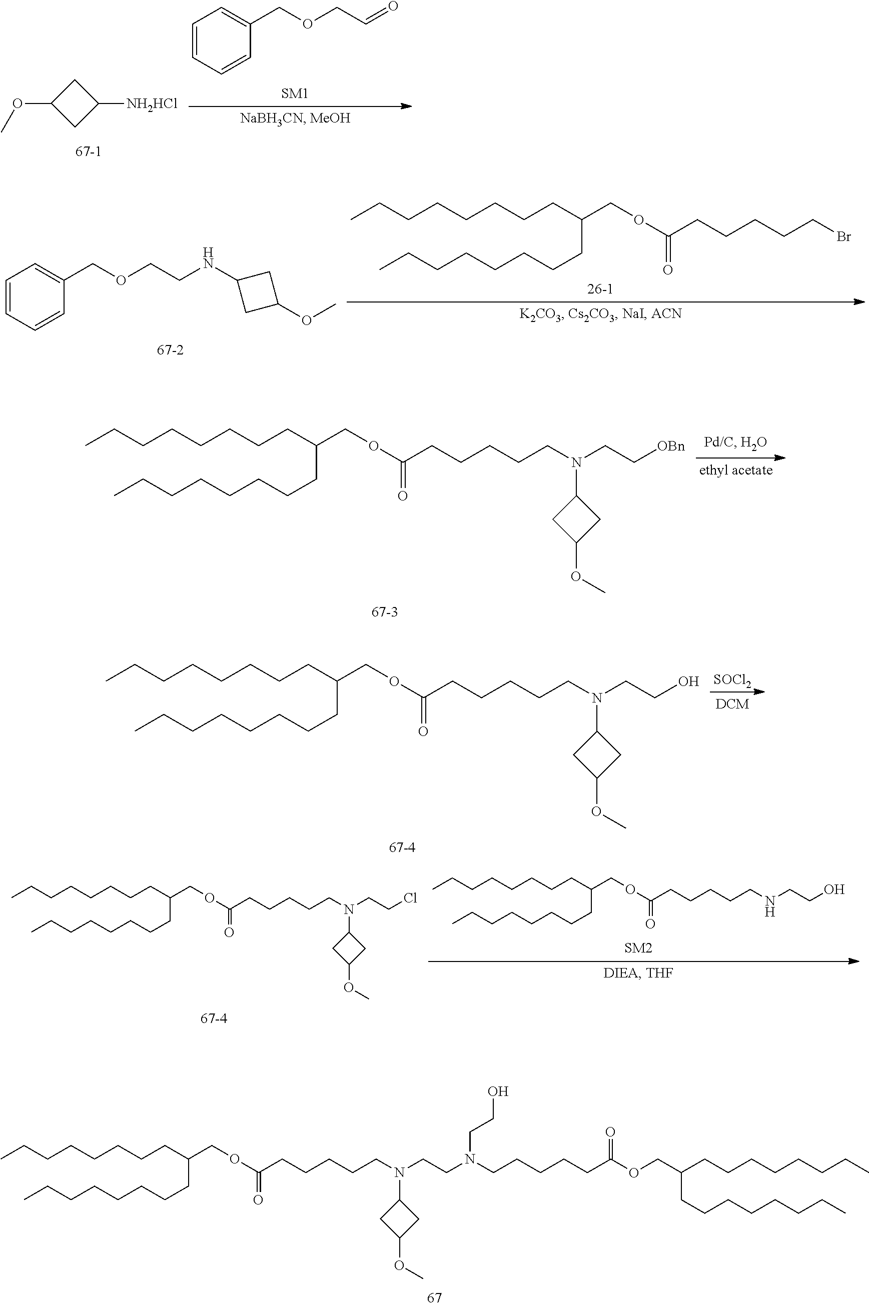

### Step 1: Preparation of Compound 67-2

A mixture of compound 67-1 (200 mg, 1.45 mmol, 1.0 eq.), compound SM1 (196 mg, 1.31 mmol, 0.9 eq.) in methanol (5 mL) was stirred at RT for 2 h. NaBH₃CN (190 mg, 3.0 mmol, 2.0 eq.) was added. The mixture was stirred at RT overnight. LCMS showed the target product. The mixture was diluted with ethyl acetate and washed with water and brine, dried, concentrated and purified by column chromatography silica gel (MeOH:DCM=0% to 10%) to give the desired product 67-2 (240 mg, 69.5% yield) as a yellow oil. LCMS: Rt: 0.730 min; MS m/z (ESI): 236.2 [M+H]⁺.

### Step 2: Preparation of Compound 67-3

A mixture of compound 67-2 (240 mg, 1.0 mmol, 1.0 eq.), compound 26-1 (550 mg, 1.2 mmol, 1.2 eq.), K₂CO₃ (420 mg, 3.0 mmol, 3.0 eq.), Cs₂CO₃ (100 mg, 0.3 mmol, 0.3 eq.) and NaI (45 mg, 0.3 mmol, 0.3 eq.) in THF (10 mL) was stirred overnight at 90° C. The mixture was concentrated under vacuum. The residual was purified by column chromatography to give the desired product 67-3 (230 mg, 38.3% yield) as brown oil. LCMS: Rt: 0.930 min; MS m/z (ESI): 602.4 [M+H]⁺.

### Step 3: Preparation of Compound 67-4

A mixture of compound 67-3 (230 mg, 0.38 mmol, 1.0 eq.), Pd/C (23 mg) in ethyl acetate (5 mL) was stirred at RT under hydrogen atmosphere overnight, LCMS showed the reaction was complete. The mixture was filtered and concentrated. The residual was used for the next step without further purification. LCMS: Rt: 1.687 min; MS m/z (ESI): 512.4 [M+H]⁺.

### Step 4: Preparation of Compound 67-5

A mixture of compound 67-4 (180 mg, 0.35 mmol, 1.0 eq.), SOCl₂ (210 mg, 1.76 mmol, 5.0 eq.) in DCM (5 mL) was stirred at 40° C. for 4 hours, LCMS showed the reaction was complete. The mixture concentrated and the residual was used for the next step without further purification.

### Step 5: Preparation of Compound 67

A mixture of compound 67-5 (220 mg (crude), 0.35 mmol, 1.0 eq.), compound SM2 (150 mg, 0.35 mmol, 1.0 eq.), DIEA (225 mg, 1.75 mmol, 5.0 eq.) in THF (5 mL) was stirred at 70° C. overnight, LCMS showed the reaction was complete. The mixture concentrated and the residue was purified by Pre-HPLC to give product 67 (102 mg, 31.6% yield). LCMS: Rt: 1.910 min; MS m/z (ESI): 921.7 [M+H]⁺.

¹H NMR (400 MHz, CDCl3) δ: 0.86-0.90 (m, 12H), 1.26-1.30 (m, 60H), 1.38-1.48 (m, 5H), 1.59-1.67 (m, 7H), 1.80-1.83 (m, 2H), 2.28-2.32 (m, 4H), 2.36-2.51 (m, 10H), 2.53-2.60 (m, 3H), 3.22-3.24 (m, 3H), 3.52-3.57 (m, 2H), 3.96 (d, J=5.6 Hz, 4H).

The following compounds were prepared in analogous fashion as Compound 67, using corresponding starting material.

| Compound | Characterization |
|---|---|
| 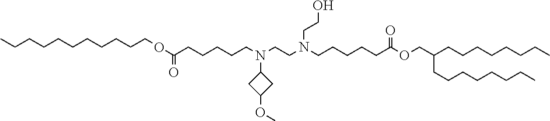 [SMILES: CCCCCCCCCCCOC(=O)CCCCCN(CCN(CCO)CCCCCC(=O)OCC(CCCCCCCC)CCCCCCCC)C1CC(OC)C1] Compound 54 | ¹H NMR (400 MHz, CDCl₃) δ: 0.86-0.90 (m, 9H), 1.26-1.30 (m, 47H), 1.38-1.48 (m, 5H), 1.60- 1.67 (m, 8H), 1.80-1.83 (m, 2H), 2.27-2.32 (m, 4H), 2.38-2.50 (m, 10H), 2.53-2.60 (m, 2H), 2.67- 2.71 (m, 1H), 3.22-3.24 (m, 3H), 3.52-3.57 (m, |
| | 3H), 3.96 (d, J = 6.0 Hz, |
| | 2H), 4.04-4.07 (m, 2H). |
| | LCMS: Rt: 1.260 min; |
| | MS m/z (ESI): 823.6 [M + H]⁺. |

### 6.27 Example 27: Preparation of Compound 68

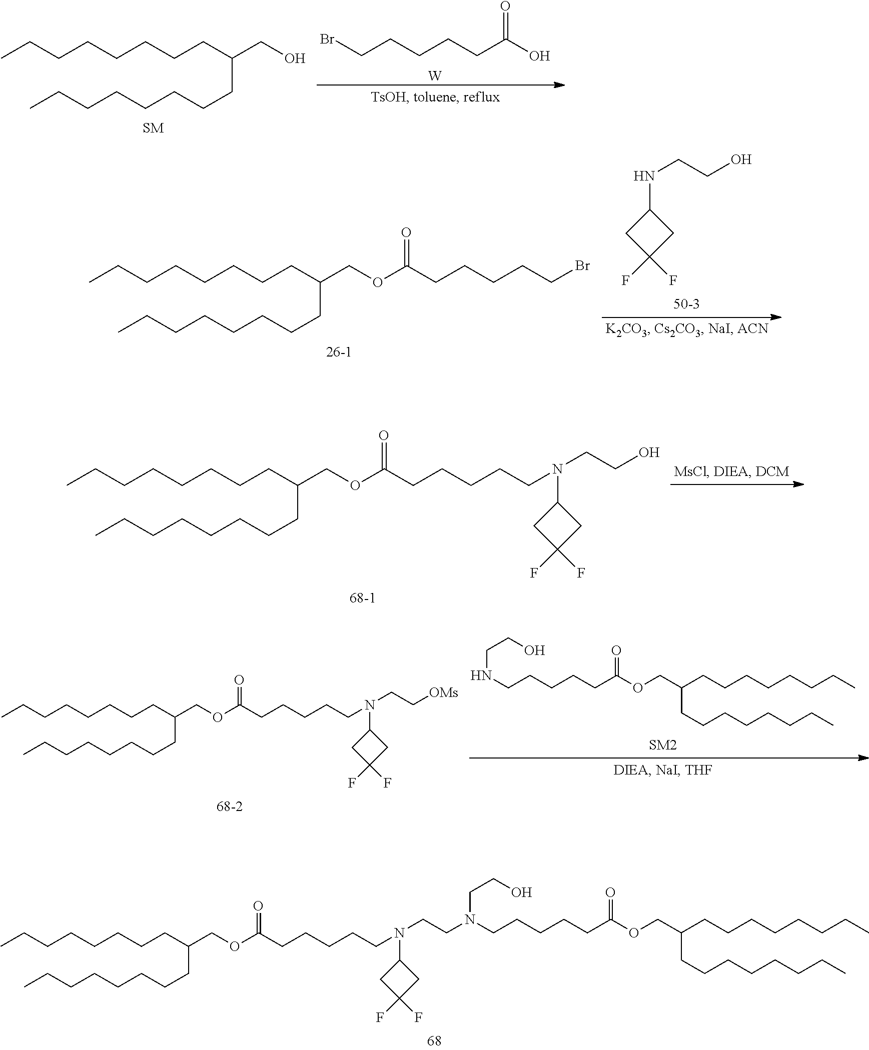

### Step 1: Preparation of Compound 26-1

To a mixture of compound SM (2.0 g, 7.394 mmol, 1.0 eq.), compound W (2.2 g, 11.09 mmol, 1.5 eq.), TsOH (500 mg) in toluene (20 mL) was stirred at reflux for 2 hours. TLC showed the reaction was complete. The mixture was evaporated under reduced pressure and FCC to get the compound 26-1 (3 g, 90.90%) as a yellow oil.

### Step 2: Preparation of Compound 68-1

To a solution of compound 26-1 (742 mg, 1.658 mmol, 1.0 eq.) in ACN (15 mL) was added compound 50-3 (0.3 g, 1.658 mmol, 1.0 eq.), K₂CO₃ (687 mg, 4.974 mmol, 3.0 eq.), Cs₂CO₃ (162 mg, 0.4974 mmol, 0.3 eq.), NaI (25 mg, 0.1658 mmol, 0.1 eq.). The reaction mixture was stirred at 80° C. for 16 hours. LCMS showed the reaction was complete. Removal of solvent, FCC to get the compound 68-1 (400 mg, 46.59%) as a yellow oil. LCMS: Rt: 1.200 min; MS m/z (ESI): 518.4 [M+H]⁺.

### Step 3: Preparation of Compound 68-2

To a mixture of compound 68-1 (200 mg, 0.3862 mmol, 1.0 eq.), DIEA (100 mg, 0.7724 mmol, 2.0 eq.) in DCM (20 mL) was added MsCl (53 mg, 0.4635 mmol, 1.2 eq.) at 0° C. under N₂. The reaction mixture was stirred at 0° C. for 1 hours. TLC showed the reaction was complete. The mixture power in water and washed with DCM. The organic was separated and dried over Na₂SO₄. Removal of solvent, FCC to get the compound 68-2 (230 mg, crude) as a yellow oil.

### Step 4: Preparation of Compound 68

To a mixture of compound 68-2 (230 mg, 0.3860 mmol, 1.0 eq.), DIEA (125 mg, 0.9648 mmol, 3.0 eq.) in THF (15 mL) was added compound SM2 (138 mg, 0.3216 mmol, 1.0 eq.), NaI (15 mg). The reaction mixture was stirred at 75° C. for 16 hours. LCMS showed the reaction was complete. After removal of solvent, the residue was purified by pre-HPLC to give the title compound (20 mg, 5.59% yield) as a yellow oil.

¹H NMR (400 MHz, CDCl₃):0.86-0.88 (m, 12H), 1.26 (s, 52H), 1.43-1.63 (m, 19H), 2.28-2.32 (m, 4H), 2.45-2.59 (m, 14H), 3.05 (s, 1H), 3.53 (s, 2H), 3.95-3.97 (m, 4H). LCMS: Rt: 1.970 min; MS m/z (ESI): 927.6 [M+H]⁺.

The following compounds were prepared in analogous fashion as Compound 68, using corresponding starting material.

| Compound | Characterization |
|---|---|
| 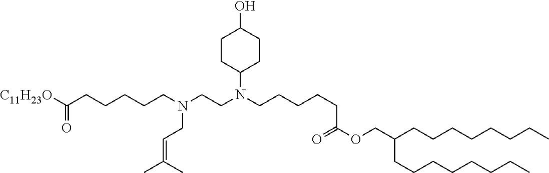 | ¹H NMR (400 MHz, CDCl3) δ: 0.73-0.93 (m, 9H), 1.17-1.38 (m, 52H), 1.52-1.68 (m, 9H), 1.69-1.87 (m, 9H), 1.93-2.10 (m, 4H), 2.22-2.34 (m, 5H), 2.35-2.65 (m, 6H), 2.78-3.11 (m, 4H), 3.46-3.63 (m, 2H), 3.91-4.01 (m, 2H), 4.02-4.12 (m, 2H). LCMS: Rt: 1.310 min; MS m/z (ESI): 861.8 [M + H]⁺. |
| Compound 125 | |

### 6.28 Example 28: Preparation of Compound 71

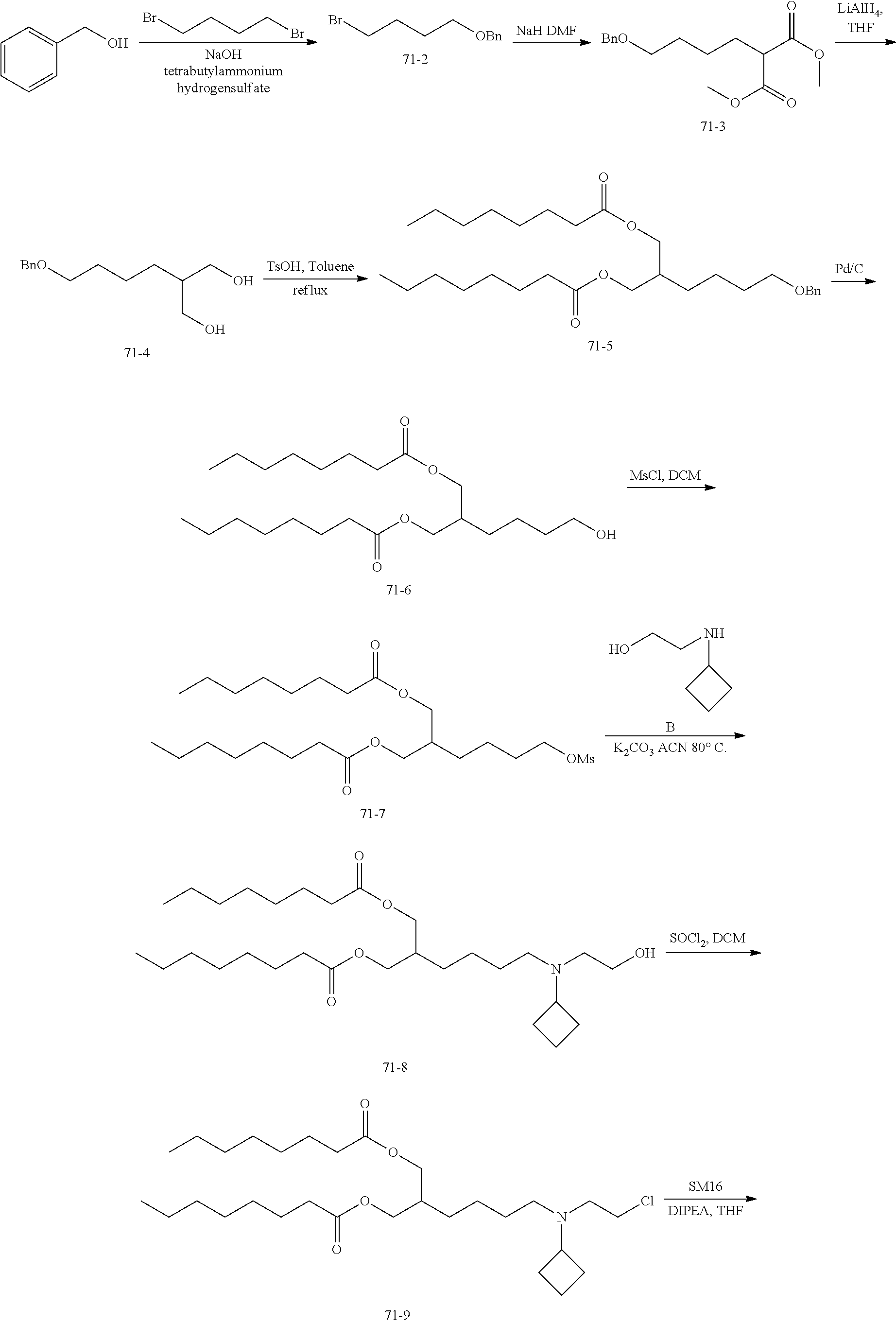

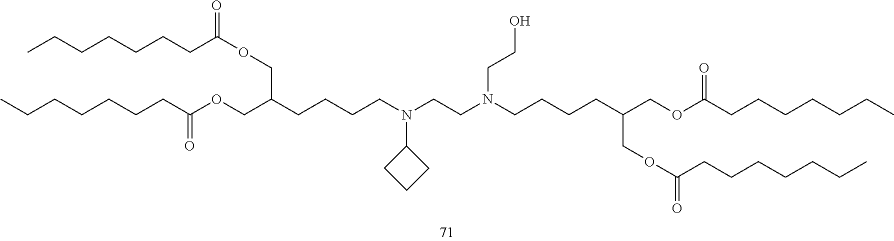 [SMILES: CCCCCCCC(=O)OCC(CCCCN(CCO)CCN(CCCCC(COC(=O)CCCCCCC)COC(=O)CCCCCCC)C1CCC1)COC(=O)CCCCCCC]

### Step 1: Preparation of Compound 71-2

To a solution of NaOH (2.0 g, 50.3 mmol, 2.5 eq.) in water (40 mL) were added compound 71-1 (2.18 g, 20.1 mmol, 1.0 eq.), 1, 4-dibromobutane (10.0 g, 46.3 mmol, 2.3 eq.) and tetrabutylammonium hydrogen sulfate (171 mg, 0.50 mmol, 0.025 eq.). The mixture was stirred at 80° C. for 16 hours. The reaction mixture was extracted with EA. The combined organic layers were washed with brine, dried over Na₂SO₄ and concentrated. The residue was purified by column chromatography on silica gel (PE/EA=50/1) to give the title compound (3.3 g, 67% yield) as colorless oil.

¹H NMR (400 MHz, CDCl₃) δ: 1.72-1.80 (m, 2H), 1.94-2.01 (m, 2H), 3.43 (t, J=6.8 Hz, 2H), 3.50 (t, J=6.2 Hz, 2H), 4.50 (s, 2H), 7.27-7.37 (m, 5H).

### Step 2: Preparation of Compound 71-3

To a suspension of NaH (653 mg, 16.3 mmol, 1.2 eq.) in THF (60 mL) was added dimethyl malonate (3.6 g, 27.2 mmol, 2.0 eq.) dropwise. Then a solution of compound 71-2 (3.3 g, 13.6 mmol, 1.0 eq.) in THF (10 mL) was added dropwise and the resulting mixture was stirred under reflux for 16 hours. After cooling to RT, the mixture was poured into water and extracted with EA. The combined organic layers were washed with brine, dried over Na₂SO₄ and concentrated. The residue was purified by column chromatography on silica gel (PE/EA=20/1-5/1) to give the title compound (3.2 g, 80% yield) as colorless oil.

### Step 3: Preparation of Compound 71-4

To a solution of LiAlH₄ (828 mg, 21.8 mmol, 2.0 eq.) in THF (40 mL) was added a solution of compound 71-3 (3.2 g, 10.9 mmol, 1.0 eq.) in THF (20 mL) dropwise. The reaction mixture was stirred at room temperature for 16 hours. The reaction mixture was cautiously with ethyl acetate and water. 6 mL of 2N NaOH aqueous solution was added. The mixture was filtered through a pad of Celite and washed with EA. The filtrate was dried over Na₂SO₄ and purified by column chromatography on silica gel (DCM/MeOH=30/1-20/1) to give the title compound (1.6 g, 62% yield) as colorless oil.

¹H NMR (400 MHz, CDCl₃) δ: 1.20-1.24 (m, 2H), 1.36-1.44 (m, 2H), 1.57-1.68 (m, 2H), 1.72-1.75 (m, 1H), 3.33 (s, 2H), 3.45-3.49 (m, 2H), 3.57-3.61 (m, 2H), 3.73-3.76 (m, 2H), 4.49 (s, 2H), 7.27-7.34 (m, 5H).

### Step 4: Preparation of Compound 71-5

To a solution of compound 71-4 (1.0 g, 4.2 mmol, 1.0 eq.) and octanoic acid (1.8 g, 12.6 mmol, 3.0 eq.) in toluene (40 mL) was added TsOH·H₂O (36 mg). The mixture was stirred under reflux through Dean-Stark trap for 4 hours. The reaction mixture was concentrated and purified by column chromatography on silica gel (PE/EA=30/1) to give the title compound (926 mg, 46% yield) as colorless oil.

¹H NMR (400 MHz, CDCl₃) δ: 0.86-0.90 (m, 6H), 1.27-1.29 (m, 13H), 1.36-1.48 (m, 4H), 1.58-1.64 (m, 9H), 1.92-2.02 (m, 1H), 2.29 (t, J=7.6 Hz, 4H), 3.46 (t, J=6.4 Hz, 2H), 4.00-4.10 (m, 4H), 4.49 (s, 2H), 7.28-7.37 (m, 5H).

### Step 5: Preparation of 71-6

To a solution of compound 71-5 (820 mg, 1.67 mmol, 1.0 eq.) in MeOH (20 mL) was added Pd/C (82 mg). The mixture was stirred at 35° C. under H₂ for 36 hours. The mixture was filtered through a pad of Celite and washed with MeOH. The filtration was concentrated to give the title compound (630 mg, 941% yield) as colorless oil.

¹H NMR (400 MHz, CDCl₃) δ: 0.86-0.90 (m, 6H), 1.27-1.39 (m, 15H), 1.41-1.51 (m, 6H), 1.58-1.65 (m, 6H), 1.96-2.05 (m, 1H), 2.30 (t, J=7.6 Hz, 4H), 3.65 (t, J=6.4 Hz, 2H), 4.02-4.11 (m, 4H).

### Step 6: Preparation of Compound 71-7

To a solution of compound 71-6 (630 mg, 1.57 mmol, 1.0 eq.) and DIPEA (406 mg, 3.14 mmol, 2.0 eq.) in DCM (15 mL) at 0° C. was added MsCl (216 mg, 1.88 mmol, 1.2 eq.). The mixture was stirred at room temperature for 2 hours. The mixture was poured into water and extracted with DCM. The combined organic layers were washed with brine, dried over Na₂SO₄ and concentrated to give the title compound (682 mg, 91% yield) as a yellow oil. It was used in the next step without further purification.

¹H NMR (400 MHz, CDCl₃) δ: 0.86-0.90 (m, 6H), 1.27-1.37 (m, 14H), 1.41-1.46 (m, 4H), 1.53-1.63 (m, 6H), 1.73-1.80 (m, 2H), 1.96-2.03 (m, 1H), 2.30 (t, J=6.2 Hz, 4H), 3.01 (s, 3H), 4.02-4.10 (m, 4H), 4.23 (t, J=6.4 Hz, 2H).

### Step 7: Preparation of Compound 71-8

To a solution of compound 71-7 (260 mg, 0.54 mmol, 1.0 eq.) and compound B (62 mg, 0.54 mmol, 1.0 eq.) in ACN (15 mL) were added K₂CO₃ (224 mg, 1.62 mmol, 3.0 eq.), Cs₂CO₃ (52 mg, 0.16 mmol, 0.3 eq.) and NaI (24 mg, 0.16 mmol, 0.3 eq.). The mixture was stirred at 80° C. for 16 hours. LCMS showed the reaction was complete. The reaction mixture was concentrated and purified by column chromatography on silica gel (DCM/MeOH=20/1) to give the title compound (182 mg, 67% yield) as a yellow oil. LCMS: Rt: 0.830 min; MS m/z (ESI): 498.4 [M+H]⁺.

### Step 8: Preparation of Compound 71-9

To a solution of compound 71-8 (180 mg, 0.36 mmol, 1.0 eq.) in DCM (10 mL) was added SOCl₂ (128 mg, 1.08 mmol, 3.0 eq.). The mixture was stirred at 30° C. for 16 hours. LCMS show the reaction was completed. The mixture was concentrated to give the title compound (185 mg, 100% yield) as a yellow oil. It was used in the next step without further purification. LCMS: Rt: 0.870 min; MS m/z (ESI): 516.3[M+H]⁺.

### Step 9: Preparation of Compound 71

To a solution of compound 71-9 (160 mg, 0.31 mmol, 1.0 eq.) and compound SM16 (138 mg, 0.31 mmol, 1.0 eq.) in THF (10 mL) were added DIPEA (120 mg, 0.93 mmol, 3.0 eq.) and NaI (14 mg, 0.093 mmol, 0.3 eq.). The mixture was stirred at 70° C. for 16 hours LCMS show the reaction was completed. The mixture was concentrated and purified by prep HPLC to give the title compound (100 mg, 35% yield) as a yellow oil.

¹H NMR (400 MHz, CDCl₃) δ:0.86-0.90 (m, 12H), 1.27-1.50 (m, 44H), 1.57-1.67 (m, 10H), 1.85-2.05 (m, 6H), 2.28-2.36 (m, 8H), 2.45-3.13 (m, 12H), 3.52-3.60 (m, 2H), 4.01-4.10 (m, 8H). LCMS: Rt: 1.110 min; MS m/z (ESI): 923.7[M+H]⁺.

The following compounds were prepared in analogous fashion as Compound 71, using corresponding starting material.

| Compound | Characterization |
|---|---|
| 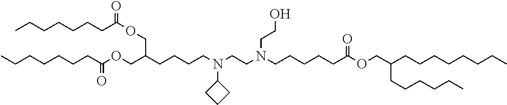 [SMILES: CCCCCCCCC(CCCCCC)COC(=O)CCCCCN(CCO)CCN(CCCCC(COC(=O)CCCCCCC)COC(=O)CCCCCCC)C1CCC1] Compound 135 | ¹H NMR (400 MHz, CDCl₃) δ: 0.86-0.90 (m, 12H), 1.27-1.35 (m, 48H), 1.43-1.53 (m, 4H), 1.56-1.70 (m, 9H), 1.92-2.08 (m, 4H), 2.30 (t, J = 7.2 Hz, 6H), 2.32-2.68 (m, 10H), 3.06-3.15 (m, 1H), 3.53-3.62 (m, 2H), 3.96-3.97 (m, 2H), 4.01-4.10 (m, 4H). LCMS: Rt: 1.160 min; MS m/z (ESI): 879.8 [M + H]⁺. |
| 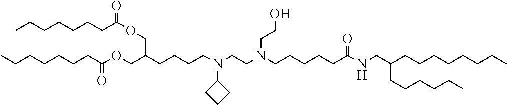 [SMILES: CCCCCCCCC(CCCCCC)CNC(=O)CCCCCN(CCO)CCN(CCCCC(COC(=O)CCCCCCC)COC(=O)CCCCCCC)C1CCC1] Compound 136 | ¹H NMR (400 MHz, CDCl₃) δ: 0.86-0.90 (m, 12H), 1.26-1.36 (m, 48H), 1.43-1.56 (m, 5H), 1.59-1.72 (m, 8H), 1.92-2.07 (m, 4H), 2.17 (t, J = 7.6 Hz, 2H), 2.30 (t, J = 7.6 Hz, 4H), 2.36-2.70 (m, 10H), 3.07- 3.10 (m, 1H), 3.13-3.19 (m, 2H), 3.53- 3.61 (m, 2H), 4.01-4.10 (m, 4H), 5.44- 5.56 (m, 1H). LCMS: Rt: 1.020 min; MS m/z (ESI): 878.8 [M + H]⁺. |
| 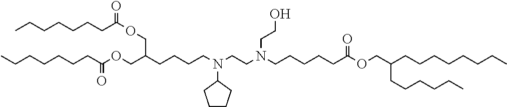 [SMILES: CCCCCCCCC(CCCCCC)COC(=O)CCCCCN(CCO)CCN(CCCCC(COC(=O)CCCCCCC)COC(=O)CCCCCCC)C1CCCC1] Compound 137 | ¹H NMR (400 MHz, CDCl₃): 0.77-0.94 (m, 12H), 1.13-1.35 (m, 42H), 1.36-1.42 (m, 4H), 1.43-1.54 (m, 4H), 1.56-1.71 (m, 10H), 1.73-1.85 (m, 3H), 2.04-2.25 (m, 4H), 2.26-2.39 (m, 7H), 2.40-2.77 (m, 8H), 2.93-3.13 (m, 2H), 3.49-3.65 (m, 2H), 3.90-4.10 (m, 6H). LCMS: Rt: 0.740 min; MS m/z (ESI): 893.8 [M + H]⁺. |
| 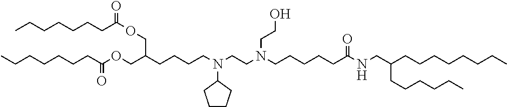 [SMILES: CCCCCCCCC(CCCCCC)CNC(=O)CCCCCN(CCO)CCN(CCCCC(COC(=O)CCCCCCC)COC(=O)CCCCCCC)C1CCCC1] Compound 138 | 1H NMR (400 MHz, CDCl₃): 0.78-0.96 (m, 12H) 1.26-1.39 (m, 45H), 1.40-1.52 (m, 9H), 1.53-1.71 (m, 10H), 1.88-2.06 (m, 2H), 2.07-2.22 (m, 3H), 2.23-2.35 (m, 5H), 2.44-2.74 (m, 10H), 3.13-3.23 (m, 2H), 3.46-3.65 (m, 2H), 3.94-4.16 (m, 4H), 5.37-5.51 (m, 1H). LCMS: Rt: 0.970 min; MS m/z (ESI): 892.8 [M + H]. |
| 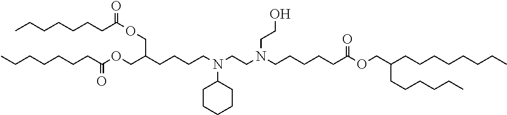 [SMILES: CCCCCCCCC(CCCCCC)COC(=O)CCCCCN(CCO)CCN(CCCCC(COC(=O)CCCCCCC)COC(=O)CCCCCCC)C1CCCCC1] | ¹H NMR (400 MHz, CDCl₃) δ: 0.86-0.90 (m, 12H), 1.27-1.36 (m, 53H), 1.42-1.52 (m, 4H), 1.58-1.64 (m, 8H), 1.66-1.87 (m, 4H), 1.93-2.00 (m, 1H), 2.28-2.33 (m, 6H), 2.39-2.66 (m, 10H), 3.48-3.59 (m, 2H), 3.96-4.00 (m, 2H), 4.01-4.10 (m, 4H). LCMS: Rt: 1.080 min; MS m/z (ESI): 907.8 [M + H]⁺. |
| Compound 139 | |
| 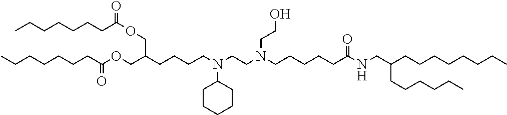 [SMILES: CCCCCCCCC(CCCCCC)CNC(=O)CCCCCN(CCO)CCN(CCCCC(COC(=O)CCCCCCC)COC(=O)CCCCCCC)C1CCCCC1] Compound 140 | ¹H NMR (400 MHz, CDCl₃) δ: 0.86-0.90 (m, 12H), 1.26-1.35 (m, 54H), 1.40-1.54 (m, 4H), 1.59-1.68 (m, 8H), 1.75-1.87 (m, 3H), 1.94-2.01 (m, 1H), 2.16-2.20 (m, 2H), 2.30 (t, J = 7.4 Hz, 4H), 2.36-2.80 (m, 10H), 3.18 (t, J = 6.0 Hz, 2H), 3.51- 3.64 (m, 2H), 4.00-4.10 (m, 4H), 5.39- 5.59 (m, 1H). LCMS: Rt: 1.137 min; MS m/z (ESI): 906.8 [M + H]⁺. |
| 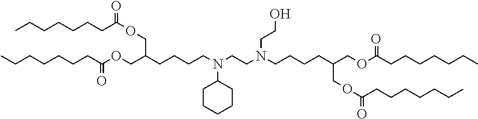 [SMILES: CCCCCCCC(=O)OCC(CCCCN(CCO)CCN(CCCCC(COC(=O)CCCCCCC)COC(=O)CCCCCCC)C1CCCCC1)COC(=O)CCCCCCC] | ¹H NMR (400 MHz, CDCl₃) δ: 0.86-0.90 (m, 12H), 1.27-1.47 (m, 46H), 1.47-1.51 (m, 3H), 1.58-1.68 (m, 9H), 1.72-1.87 (m, 4H), 1.93-2.13 (m, 2H), 2.28-2.36 (m, 8H), 2.44-3.21 (m, 12H), 3.46-3.76 (m, 2H), 4.01-4.10 (m, 8H). LCMS: Rt: 1.077 min; MS m/z (ESI): 951.7 [M + H]⁺. |
| Compound 141 | |
| 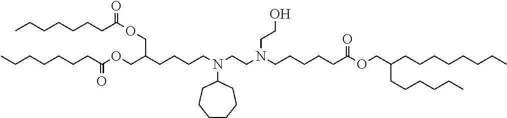 [SMILES: CCCCCCCCC(CCCCCC)COC(=O)CCCCCN(CCO)CCN(CCCCC(COC(=O)CCCCCCC)COC(=O)CCCCCCC)C1CCCCCC1] | ¹H NMR (400 MHz, CDCl₃) δ: 0.86-0.90 (m, 12H), 1.27-1.40 (m, 59H), 1.43-1.71 (m, 10H), 1.75-1.84 (m, 2H), 1.94-2.02 (m, 1H), 2.28-2.35 (m, 6H), 2.34-2.46 (m, 7H), 2.54-2.62 (m, 2H), 2.64-2.72 (m, 1H), 3.48-3.56 (m, 2H), 3.96-3.97 (m, 2H), 4.00-4.10 (m, 4H). LCMS: Rt: 1.367 min; MS m/z (ESI): 921.8 [M + H]⁺. |
| Compound 142 | |
| 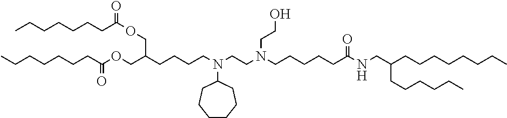 [SMILES: CCCCCCCCC(CCCCCC)CNC(=O)CCCCCN(CCO)CCN(CCCCC(COC(=O)CCCCCCC)COC(=O)CCCCCCC)C1CCCCCC1] Compound 143 | ¹H NMR (400 MHz, CDCl₃) δ: 0.86-0.90 (m, 12H), 1.26-1.52 (m, 61H), 1.56-1.71 (m, 8H), 1.74-1.84 (m, 2H), 1.94-2.02 (m, 1H), 2.14-2.19 (m, 2H), 2.28-2.32 (m, 4H), 2.34-2.53 (m, 7H), 2.55-2.61 (m, 2H), 2.64-2.72 (m, 1H), 3.17-3.19 (m, 2H), 3.47-3.58 (m, 2H), 4.00-4.10 (m, 4H), 5.37-5.47 (m, 1H). LCMS: Rt: 1.187 min; MS m/z (ESI): 920.9 [M + H]⁺. |
| 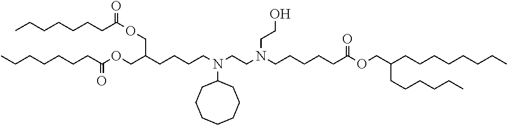 [SMILES: CCCCCCCCC(CCCCCC)COC(=O)CCCCCN(CCO)CCN(CCCCC(COC(=O)CCCCCCC)COC(=O)CCCCCCC)C1CCCCCCC1] Compound 144 | ¹H NMR (400 MHz, CDCl₃) δ: 0.86-0.90 (m, 12H), 1.27-1.36 (m, 46H), 1.38-1.51 (m, 12H), 1.54-1.61 (m, 10H), 1.63-1.68 (m, 4H), 1.94-2.02 (m, 1H), 2.28-2.44 (m, 10H), 2.45-2.53 (m, 4H), 2.56-2.62 (m, 2H), 2.76-2.84 (m, 1H), 3.49-3.58 (m, 2H), 3.96-3.97 (m, 2H), 4.00-4.10 (m, 4H). LCMS: Rt: 1.330 min; MS m/z (ESI): 935.8 [M + H]⁺. |
| 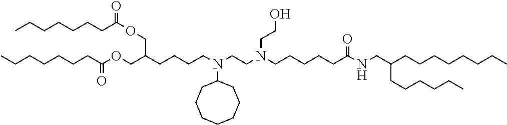 [SMILES: CCCCCCCCC(CCCCCC)CNC(=O)CCCCCN(CCO)CCN(CCCCC(COC(=O)CCCCCCC)COC(=O)CCCCCCC)C1CCCCCCC1] Compound 145 | ¹H NMR (400 MHz, CDCl₃) δ: 0.86-0.90 (m, 12H), 1.26-1.35 (m, 53H), 1.42-1.52 (m, 4H), 1.58-1.67 (m, 11H), 1.71-1.75 (m, 3H), 1.94-2.02 (m, 1H), 2.28-2.32 (m, 6H), 2.37-2.64 (m, 10H), 3.03-3.11 (m, 2H), 3.48-3.57 (m, 2H), 3.95-3.97 (m, 2H), 4.00-4.10 (m, 4H), 5.23-5.28 (m, 1H). LCMS: Rt: 1.180 min; MS m/z (ESI): 934.8 [M + H]⁺. |
| 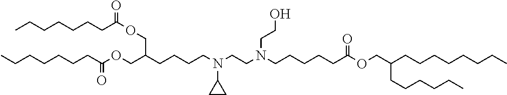 [SMILES: CCCCCCCCC(CCCCCC)COC(=O)CCCCCN(CCO)CCN(CCCCC(COC(=O)CCCCCCC)COC(=O)CCCCCCC)C1CC1] Compound 154 | ¹H NMR (400 MHz, CDCl₃) δ: 0.41-0.51 (m, 2H), 0.83-0.94 (m, 12H), 1.04-1.16 (m, 9H), 1.17-1.38 (m, 34H), 1.39-1.49 (m, 4H), 1.50-1.70 (m, 10H), 1.71-1.80 (m, 10H), 1.90-2.05 (m, 1H), 2.24-2.37 (m, 5H), 2.47-2.68 (m, 5H), 3.07-3.22 (m, 1H), 3.45-3.57 (m, 1H), 3.92-4.13 (m, 6H). LCMS: Rt: 1.020 min; MS m/z (ESI): 865.8 [M + H]⁺. |
| 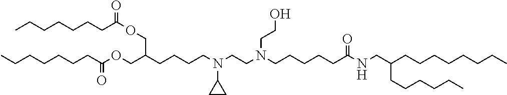 [SMILES: CCCCCCCCC(CCCCCC)CNC(=O)CCCCCN(CCO)CCN(CCCCC(COC(=O)CCCCCCC)COC(=O)CCCCCCC)C1CC1] Compound 155 | ¹H NMR (400 MHz, CDCl₃) δ: 0.37-0.59 (m, 4H), 0.81-0.96 (m, 12H), 1.17-1.42 (m, 44H), 1.43-1.72 (m, 12H), 1.73-2.06 (m, 8H), 2.15-2.23 (m, 2H), 2.24-2.37 (m, 4H), 2.38-2.77 (m, 7H), 3.12-3.26 (m, 3H), 3.40-3.58 (m, 1H), 3.95-4.15 (m, 4H). LCMS: Rt: 1.000 min; MS m/z (ESI): 864.8 [M + H]⁺. |

### 6.29 Example 29: Preparation of Compound 72

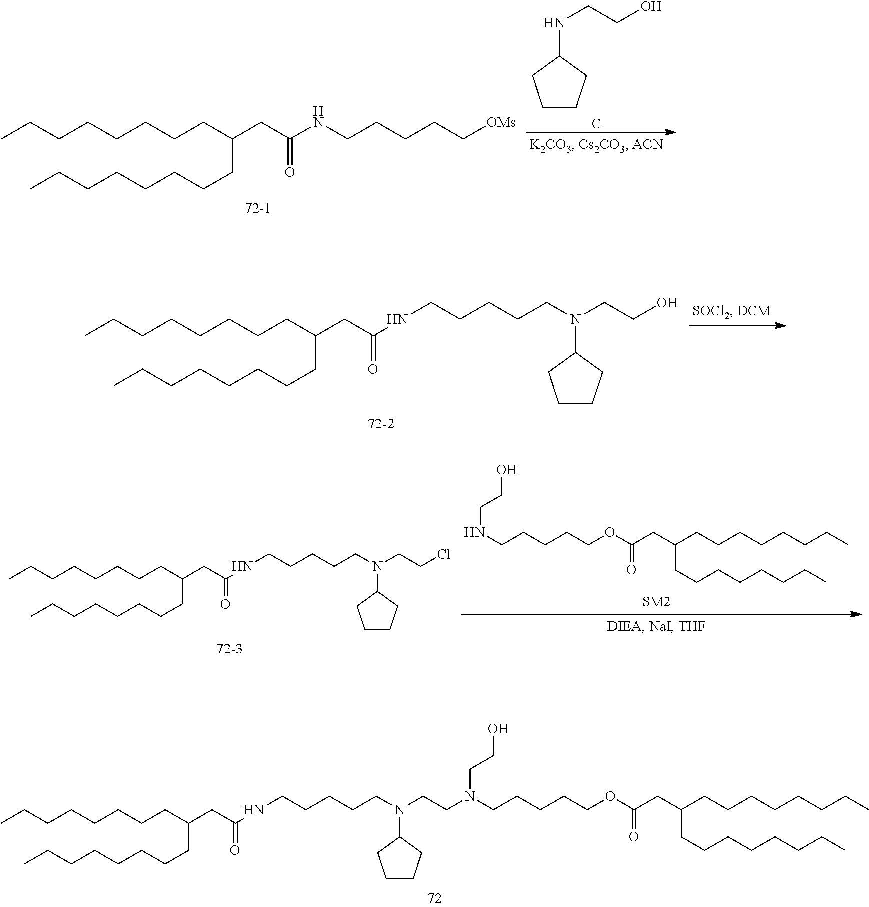

### Step 1: Preparation of Compound 72-2

A mixture of compound 72-1 (400 mg, 0.86 mmol, 1 eq.), compound C (167 mg, 1.3 mmol, 1.5 eq.) and K₂CO₃ (359 mg, 2.6 mmol, 3 eq.), Cs₂CO₃ (10 mg, 0.03 mmol, 0.03 eq.) in ACN (10 mL) was stirred overnight at 80° C. The mixture was concentrated and purified by column chromatography silica gel (MeOH:DCM=0% to 3%) to give the desired product 72-2 (106 mg, 24.7% yield) as a yellow oil. LCMS: Rt: 0.810 min; MS m/z (ESI): 495.4 [M+H]⁺.

### Step 2: Preparation of Compound 72-3

A mixture of 72-2 (106 mg, 0.2 mmol, 1 eq.) and SOCl₂ (77 mg, 0.6 mmol, 3 eq.) in DCM (5 mL) was stirred overnight at 35° C. The mixture diluted with water, extracted over ethyl acetate, after dried and concentrated to give the desired product 72-3 (117 mg, crude) as a yellow oil. LCMS: Rt: 0.880 min; MS m/z (ESI): 513.4 [M+H]⁺.

### Step 3: Preparation of Compound 72

A mixture of compound 72-3 (91 mg, 0.19 mmol, 1.0 eq.), compound SM2 (100 mg, 0.23 mmol, 1.2 eq.), K₂CO₃ (79 mg, 0.57 mmol, 3.0 eq.), Cs₂CO₃ (3 mg, 0.01 mmol, 0.03 eq.), NaI (15 mg, 0.10 mmol, 0.5 eq.) in ACN (5 mL) was stirred overnight at 80° C. The mixture was concentrated under vacuum. The residual was purified by prep-HPLC to give the desired product 72 (31 mg, 18.1% yield) as a yellow oil. LCMS: Rt: 1.510 min; MS m/z (ESI): 904.7 [M+H]⁺.

¹H NMR (400 MHz, CDCl₃) δ: 0.81-0.93 (m, 12H), 1.07-1.38 (m, 62H), 1.39-1.57 (m, 9H), 1.58-1.90 (m, 11H), 1.96-2.10 (m, 3H), 2.16-2.26 (m, 2H), 2.42-2.68 (m, 8H), 3.18-3.32 (m, 2H), 3.49-3.61 (m, 2H), 3.99-4.12 (m, 2H).

The following compounds were prepared in analogous fashion as Compound 72, using corresponding starting material.

| Compound | Characterization |
|---|---|
| 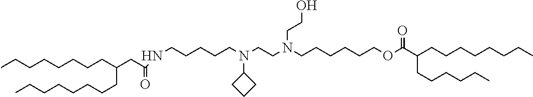 [SMILES: CCCCCCCCC(CCCCCCCC)CC(=O)NCCCCCN(CCN(CCO)CCCCCCOC(=O)C(CCCCCC)CCCCCCCC)C1CCC1] Compound 70 | ¹H NMR (400 MHz, CDCl₃) δ: 0.75-0.94 (m, 12H), 1.12-1.26 (m, 55H), 1.39-1.50 (m, 8H), 1.51-1.71 (m, 10H), 1.90-2.10 (m, 4H), 2.23-2.36 (m, 3H), 2.37-2.39 (m, 2H), 2.42-2.54 (m, 5H), 2.59-2.68 (m, 3H), 3.17-3.24 (m, 2H), 3.47-3.65 (m, 1H), 3.95-4.11 (m, 2H). LCMS: Rt: 1.270 min; MS m/z (ESI): 862.7 [M + H]⁺. |
| 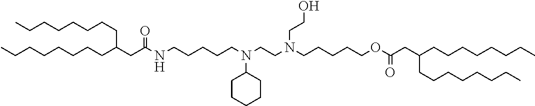 [SMILES: CCCCCCCCC(CCCCCCCC)CC(=O)NCCCCCN(CCN(CCO)CCCCCOC(=O)CC(CCCCCCCC)CCCCCCCC)C1CCCCC1] Compound 73 | 1H NMR (400 MHz, CDCl₃): 0.86-0.89 (m, 12H), 1.26 (s, 64H), 1.45-1.52 (m, 6H), 1.61-1.66 (m, 4H), 1.78-1.85 (m, 7H), 2.06 (d, J = 6.8 Hz, 2H), 2.22 (d, J = 6.8 Hz, 2H), 2.45-2.60 (m, 11H), 3.22- 3.26 (m, 2H), 3.53-3.55 (m, 2H), 4.04- 4.07 (m, 2H), 5.92 (s, 1H). LCMS: Rt: 1.490 min; MS m/z (ESI): 918.7 [M + H]⁺. |
| 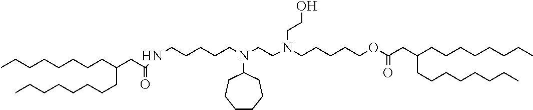 [SMILES: CCCCCCCCC(CCCCCCCC)CC(=O)NCCCCCN(CCN(CCO)CCCCCOC(=O)CC(CCCCCCCC)CCCCCCCC)C1CCCCCC1] Compound 74 | ¹H NMR (400 MHz, CDCl₃) δ: 0.82-0.92 (m, 12H), 1.02-1.36 (m, 64H), 1.38-1.57 (m, 13H), 1.59-1.74 (m, 4H), 1.76-1.89 (m, 4H), 2.03-2.11 (m, 2H), 2.18-2.26 (m, 2H), 2.35-2.73 (m, 10H), 3.19-3.29 (m, 2H), 3.49-3.60 (m, 2H), 4.01-4.09 (m, 2H). LCMS: Rt: 1.750 min; MS m/z (ESI): 932.7 [M + H]⁺. |
| 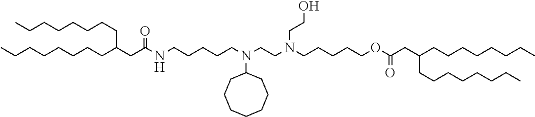 [SMILES: CCCCCCCCC(CCCCCCCC)CC(=O)NCCCCCN(CCN(CCO)CCCCCOC(=O)CC(CCCCCCCC)CCCCCCCC)C1CCCCCCC1] Compound 75 | ¹H NMR (400 MHz, CDCl₃): 0.86-0.90 (m, 12H), 1.26 (s, 64H), 1.43-1.51 (m, 10H), 1.60-1.75 (m, 9H), 1.84 (s, 2H), 2.07 (d, J = 7.2 Hz, 2 H), 2.22 (d, J = 6.8 Hz, 2H), 2.40-2.61 (m, 10H), 2.86 (s, 1H), 3.22-3.27 (m, 2H), 3.53-3.56 (m, 2H), 4.04-4.07 (m, 2H), 5.92 (s, 1H). LCMS: Rt: 1.810 min; MS m/z (ESI): 946.8 [M + H]⁺. |

### 6.30 Example 30: Preparation of Compound 76

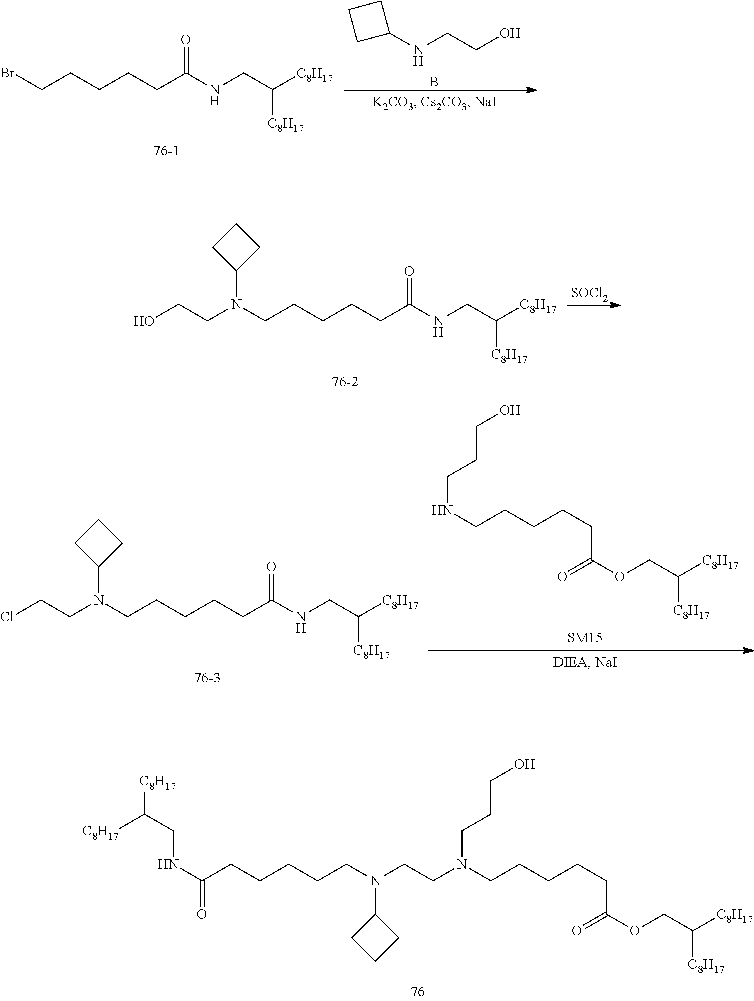

### Step 1: Preparation of Compound 76-2

To a solution of compound 76-1 (800 mg, 1.79 mmol, 1.0 eq.) in ACN (50 mL) was added compound B (210 mg, 1.79 mmol, 1.0 eq.), K₂CO₃ (750 mg, 5.37 mmol, 3.0 eq.), Cs₂CO₃ (180 mg, 0.54 mmol, 0.3 eq.) and NaI (80 mg, 0.54 mmol, 0.3 eq.). The reaction mixture was stirred at 80° C. for 10 hours. LCMS showed the reaction was complete. Removal of solvent, FCC to get the compound 76-1 (700 mg, 81%). LCMS: Rt: 0.870 min; MS m/z (ESI): 481.4 [M+H]⁺.

### Step 2: Preparation of Compound 76-3

To a solution of compound 76-2 (200 mg, 0.41 mmol, 1.0 eq.) in DCM (10 mL) was added SOCl₂ (150 mg, 1.25 mmol, 3.0 eq.). The reaction mixture was stirred at 35° C. for 10 hours. LCMS showed the reaction was complete. Removal of solvent to get the compound 76-3 (207 mg, 100% yield) as a yellow oil. LCMS: Rt: 0.440 min; MS m/z (ESI): 499.3 [M+H]

### Step 3: Preparation of Compound 76

To a mixture of compound 76-3 (120 mg, 0.23 mmol, 1.0 eq.), DIEA (90 mg, 0.68 mmol, 3.0 eq.) in THF (10 mL) was added compound SM15 (100 mg, 0.23 mmol, 1.0 eq.), NaI (35 mg). The reaction mixture was stirred at 70° C. for 10 hours. LCMS showed the reaction was complete. After removal of solvent, the residue was purified by pre-HPLC to give the title compound (35 mg, 17% yield) as a yellow oil.

¹H NMR (400 MHz, CDCl₃): 0.87 (t, J=8 Hz, 12H), 1.26-2.00 (m, 79H), 2.15-2.60 (m, 14H), 3.16-3.19 (m, 3H), 3.75-3.77 (m, 2H), 3.95-3.97 (m, 2H), 5.89 (brs, 1H). LCMS: Rt: 0.600 min; MS m/z (ESI): 904.7 [M+H]⁺.

The following compounds were prepared in analogous fashion as Compound 76, using corresponding starting material.

| Compound | Characterization |
|---|---|
| 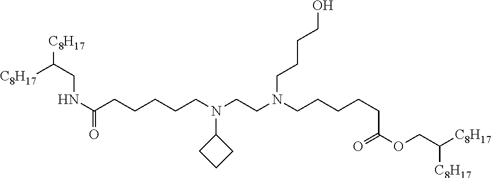 | ¹H NMR (400 MHz, CDCl₃): 0.87 (t, J = 8 Hz, 12H), 1.26-2.00 (m, 81H), 2.15-2.46 (m, 14H), 3.16-3.19 (m, 3H), 3.54-3.55 (m, 2H), 3.95-3.97 (m, 2H), 5.67 (brs, 1H). LCMS: Rt: 0.560 min; MS m/z (ESI): 918.7 [M + H]⁺. |
| Compound 77 | |
| 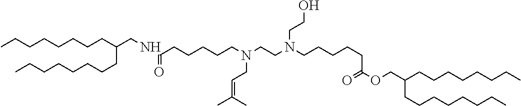 [SMILES: CCCCCCCCC(CCCCCCCC)CNC(=O)CCCCCN(CC=C(C)C)CCN(CCO)CCCCCC(=O)OCC(CCCCCCCC)CCCCCCCC] Compound 121 | ¹H NMR (400 MHz, CDCl₃) δ: 0.82-0.93 (m, 12H), 1.16-1.36 (m, 57H), 1.40-1.55 (m, 5H), 1.63-1.69 (m, 14H), 1.74 (s, 5H), 2.12-2.24 (m, 2H), 2.25-2.35 (m, 2H), 2.37-2.66 (m, 8H), 3.01-3.23 (m, 3H), 3.53 (s, 2H), 3.88-4.01 (m, 2H), 5.17-5.29 (m, 1H). LCMS: Rt: 1.880 min; MS m/z (ESI): 904.8 [M + H]⁺. |
| 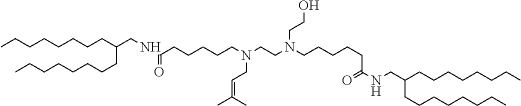 [SMILES: CCCCCCCCC(CCCCCCCC)CNC(=O)CCCCCN(CC=C(C)C)CCN(CCO)CCCCCC(=O)NCC(CCCCCCCC)CCCCCCCC] | ¹H NMR (400 MHz, CDCl₃) δ: 0.81-0.92 (m, 12H), 1.13-1.37 (m, 60H), 1.40-1.54 (m, 6H), 1.56-1.77 (m, 15H), 2.12-2.22 (m, 4H), 2.36-2.61 (m, 9H), 3.01-3.22 (m, 5H), 3.44-3.58 (m, 2H), 519-5.28 (m, 1H). LCMS: Rt: 1.300 min; MS m/z (ESI): 903.9 [M + H]⁺. |
| Compound 122 | |

### 6.31 Example 31: Preparation of Compound 78

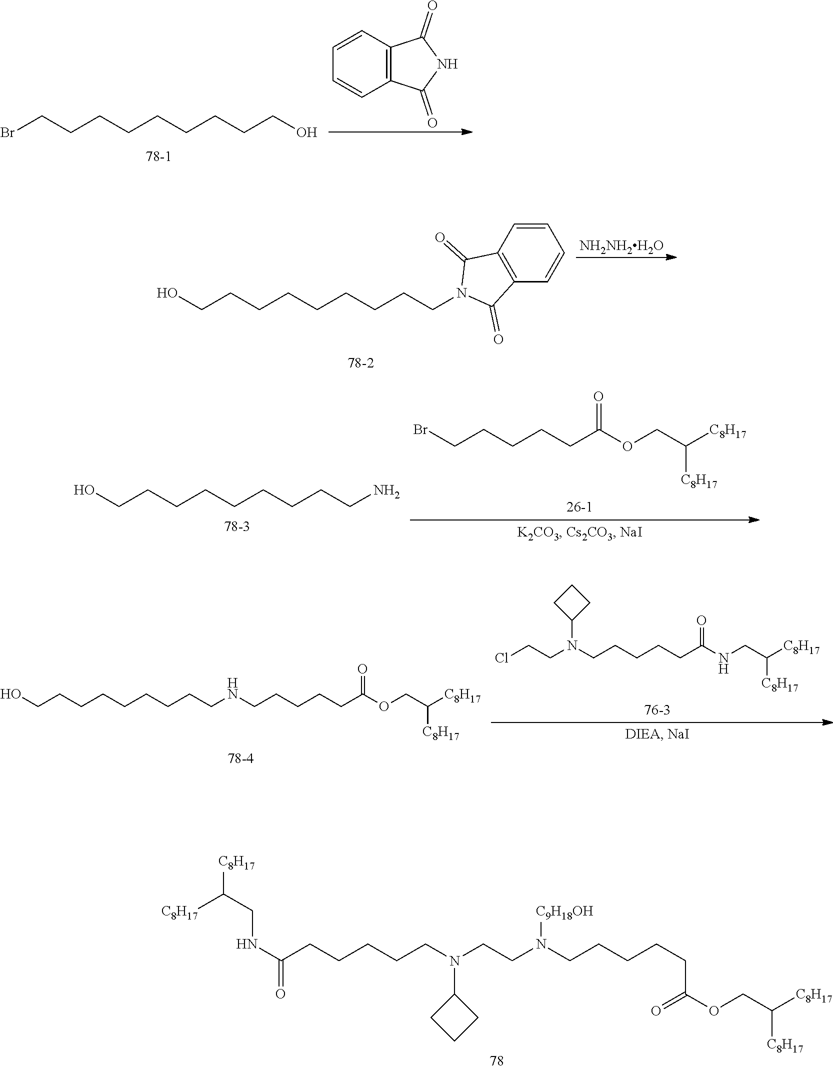

### Step 1: Preparation of Compound 78-2

To a solution of compound 78-1 (3.0 g, 13.44 mmol, 1.0 eq.) in DMF (100 mL) was added potassium 1,3-dioxoisoindolin-2-ide (4.98 g, 26.89 mmol, 2.0 eq.). The reaction mixture was stirred at 100° C. for 10 hours. LCMS showed the reaction was complete. Removal of solvent, FCC to get the compound 78-2 (2.0 g, 53%). LCMS: Rt: 1.120 min; MS m/z (ESI): 290.1 [M+H]⁺.

### Step 2: Preparation of Compound 78-3

To a solution of compound 78-2 (2.0 g, 6.91 mmol, 1.0 eq.) in EtOH (50 mL) was added NH₂NH₂·H₂O (0.7 g, 13.82 mmol, 2.0 eq.). The reaction mixture was stirred at 90° C. for 10 hours. LCMS showed the reaction was complete. Removal of solvent, FCC to get the compound 78-3 (0.5 g, 45%). LCMS: Rt: 0.590 min; MS m/z (ESI): 160.2 [M+H]⁺.

### Step 3: Preparation of Compound 78-4

To a solution of compound 26-1 (1.4 g, 3.14 mmol, 1.0 eq.) in ACN (50 mL) was added compound 78-3 (500 mg, 3.14 mmol, 1.0 eq.), K₂CO₃ (1.3 g, 9.42 mmol, 3.0 eq.), Cs₂CO₃ (300 mg, 0.94 mmol, 0.3 eq.) and NaI (140 mg, 0.94 mmol, 0.3 eq.). The reaction mixture was stirred at 80° C. for 10 hours. LCMS showed the reaction was complete. Removal of solvent, FCC to get the compound 78-4 (165 mg, 10%). LCMS: Rt: 0.920 min; MS m/z (ESI): 526.4 [M+H]⁺.

### Step 4: Preparation of Compound 78

To a mixture of compound 78-4 (140 mg, 0.27 mmol, 1.0 eq.), DIEA (100 mg, 0.80 mmol, 3.0 eq.) in THF (10 mL) was added compound 76-3 (140 mg, 0.27 mmol, 1.0 eq.), NaI (40 mg). The reaction mixture was stirred at 70° C. for 10 hours. LCMS showed the reaction was complete. After removal of solvent, the residue was purified by pre-HPLC to give the title compound (20 mg, 7% yield) as a yellow oil. ¹H NMR (400 MHz, CDCl₃): 0.87 (t, J=8 Hz, 12H), 1.26-1.98 (m, 91H), 2.08-2.41 (m, 14H), 3.00-3.09 (m, 3H), 3.55-3.58 (m, 2H), 3.88-3.90 (m, 2H), 5.89 (brs, 1H). LCMS: Rt: 0.613 min; MS m/z (ESI): 988.7[M+H]⁺.

### 6.32 Example 32: Preparation of Compound 79

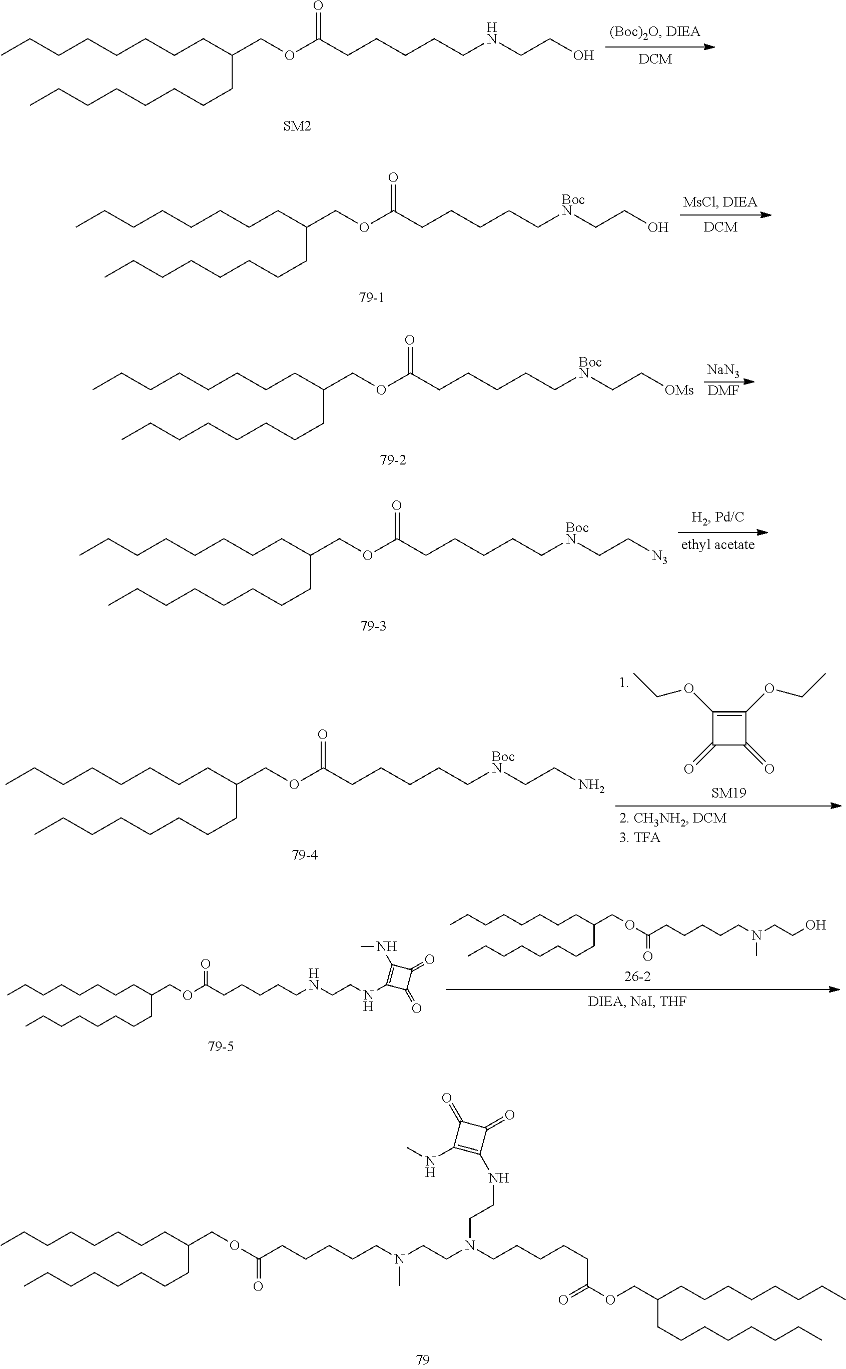

### Step 1: Preparation of Compound 79-1

A mixture of compound SM2 (500 mg, 1.2 mmol, 1.0 eq.), DIEA (300 mg, 2.3 mmol, 2.0 eq.) in DCM (5 mL) was added Boc₂O (306 mg, 1.4 mmol, 1.2 eq.). The mixture was stirred for 30 min at RT. TLC showed the reaction was complete. The mixture was concentrated and the residue was purified by column chromatography to give product 79-1 (520 mg, 91% yield) as colorless oil.

### Step 2: Preparation of Compound 79-2

A mixture of compound 79-1 (520 mg, 1.0 mmol, 1.0 eq.), DIEA (260 mg, 2.0 mmol, 2.0 eq.) in DCM (5 mL) was added MsCl (140 mg, 1.2 mmol, 1.2 eq.). The mixture was stirred for 30 min at RT. TLC showed the reaction was complete. The mixture was diluted with water and brine, dried, concentrated and the residue was used for the next step without further purification.

### Step 3: Preparation of Compound 79-3

A mixture of compound 79-2 (560 mg (crude), 1.0 mmol, 1.0 eq.), NaN₃ (100 mg, 1.5 mmol, 1.5 eq.) in DMF (10 mL) was stirred at 100° C. overnight, TLC showed the reaction was complete. The mixture was diluted with ethyl acetate and washed with water and brine, dried and concentrated. The residue was purified by column chromatography to give compound 79-3 (180 mg, 36% yield) as colorless oil.

### Step 4: Preparation of Compound 79-4

A mixture of compound 79-3 (180 mg, 0.33 mmol, 1.0 eq.), Pd/C (18 mg) in ethyl acetate (5 mL) was stirred at RT overnight, LCMS showed the reaction was complete. The mixture was filtered and concentrated. The residue was used for the next step without further purification. LCMS: Rt: 0.900 min; MS m/z (ESI): 527.4 [M+H]⁺.

### Step 5: Preparation of Compound 79-5

A mixture of compound 79-4 (170 mg, 0.33 mmol, 1.0 eq.) and compound SM19 (170 mg, 1.0 mmol, 3.0 eq.) in DCM (5 mL) was stirred at RT overnight, Then methylamine was added. The mixture was stirred for 24 hours. TFA (5 mL) was added. The mixture was stirred for 2 hours and concentrated. The residue was diluted with ethyl acetate and washed with saturated NaHCO₃ (aq.), dried, concentrated. The residue was used for the next step without further purification. LCMS: Rt: 0.900 min; MS m/z (ESI): 536.4 [M+H]⁺.

### Step 6: Preparation of Compound 79

A mixture of compound 79-5 (90 mg (crude), 0.17 mmol, 1.0 eq.), compound 26-2 (180 mg, 0.34 mmol, 2.0 eq.), DIEA (110 mg, 0.85 mmol, 5.0 eq.) in THF (5 mL) was stirred at 70° C. overnight, LCMS showed the reaction was complete. The mixture concentrated and the residue was purified by prep-HPLC to give product compound 79 (26 mg, 18% yield).

¹H NMR (400 MHz, CDCl₃) δ: 0.86-0.90 (m, 12H), 1.26-1.30 (m, 59H), 1.43-1.48 (m, 4H), 1.57-1.65 (m, 6H), 1.93 (s, 3H), 2.21 (s, 3H), 2.29-2.48 (m, 10H), 2.54 (s, 2H), 2.61-2.63 (m, 2H), 3.30 (d, J=4.2 Hz, 3H), 3.67 (d, J=5.6 Hz, 2H), 3.94-3.97 (m, 4H). LCMS: Rt: 2.47 min; MS m/z (ESI): 959.7 [M+H]⁺.

### 6.33 Example 33: Preparation of Compound 80

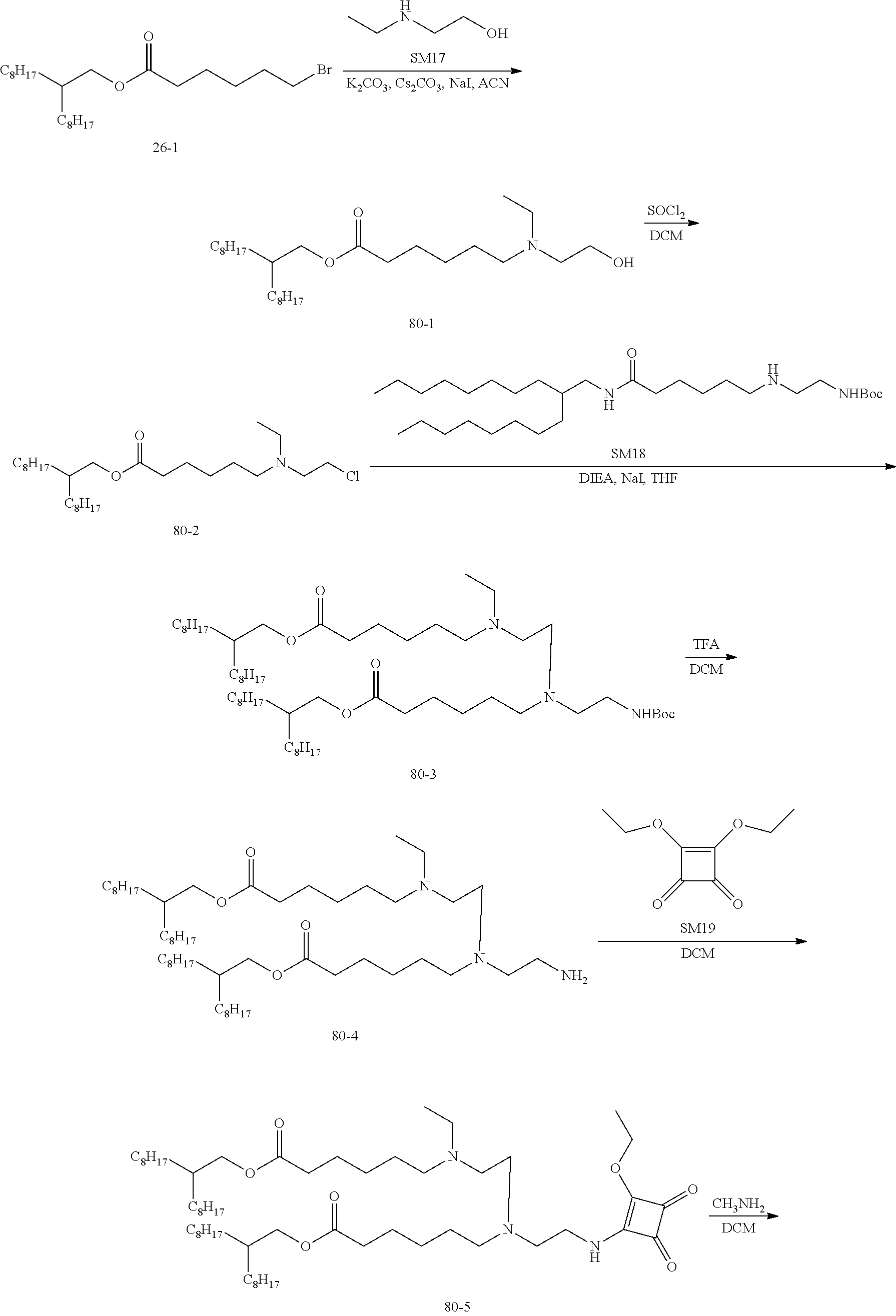

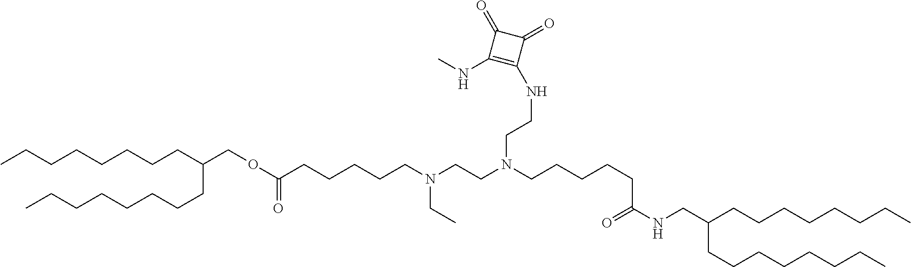 [SMILES: CCCCCCCCC(CCCCCCCC)CNC(=O)CCCCCN(CCNc1c(NC)c(=O)c1=O)CCN(CC)CCCCCC(=O)OCC(CCCCCCCC)CCCCCCCC]

### Step 1: Preparation of Compound 80-1

To a solution of compound 26-1 (600 mg, 1.34 mmol, 1.0 eq.) and compound SM17 (143 mg, 1.61 mmol, 1.2 eq.) was dissolved in ACN (10 mL) was added Cs₂CO₃ (130 mg, 0.40 mmol, 0.3 eq.), K₂CO₃ (555 mg, 4.02 mmol, 3.0 eq.) and NaI (18 mg, 0.13 mmol, 0.1 eq.) at 0° C. The mixture was stirred for 16 hours at 85° C. LCMS show the reaction was completed, the mixture was evaporated under reduced pressure and purified by FCC (DCM/MeOH=1/0-20/1) to provide compound 80-1 (520 mg, 86% yield) as a yellow oil. LCMS: Rt: 0.890 min; MS m/z (ESI): 456.4 [M+H]⁺.

### Step 2: Preparation of Compound 80-2

To a solution of compound 80-1 (200 mg, 0.44 mmol, 1.0 eq.) was dissolved in DCM (10 mL) was added SOCl₂ (264 mg, 2.20 mmol, 5.0 eq.) at RT. The mixture was stirred for 16 hours at 35° C. LCMS show the reaction was completed, the mixture was evaporated under reduced pressure to provide compound 80-2 (200 mg, crude) as a yellow oil. LCMS: Rt: 0.940 min; MS m/z (ESI): 474.3 [M+H]⁺.

### Step 3: Preparation of Compound 80-3

To a solution of compound 80-2 (300 mg, 0.63 mmol, 1.2 eq.) and compound SM18 (280 mg, 0.53 mmol, 1.0 eq.) in THF (10 mL) was added DIEA (340 mg, 2.65 mmol, 5.0 eq.) and NaI (7 mg, 0.05 mmol, 0.1 eq.) at 0° C. The mixture was stirred for 16 hours at 75° C. LCMS show the reaction was completed, the mixture was evaporated under reduced pressured purified with pre-HPLC to provide compound 80-3 (300 mg, 59% yield) as colorless oil. LCMS: Rt: 1.710 min; MS m/z (ESI): 963.7 [M+H]⁺.

### Step 4: Preparation of Compound 80-4

To a solution of compound 80-3 (200 mg, 0.21 mmol, 1.0 eq.) in DCM (4 mL), TFA (1 mL) was added at RT. The mixture was stirred for 16 hours at RT. LCMS show the reaction was completed, the mixture was evaporated under reduced pressure to provide compound 80-4 (200 mg, crude) as colorless oil. LCMS: Rt: 0.850 min; MS m/z (ESI): 863.7 [M+H]⁺.

### Step 5: Preparation of Compound 80-5

To a solution of compound 80-4 (200 mg, 0.23 mmol, 1.0 eq.) was dissolved in DCM (5 mL) was added compound SM19 (50 mg, 0.28 mmol, 1.2 eq.) at RT. The mixture was stirred for 16 hours at 40° C. LCMS show the reaction was completed, the mixture was evaporated under reduced pressure to provide compound 80-5 (200 mg, crude) as a yellow oil. LCMS: Rt: 1.310 min; MS m/z (ESI): 987.7 [M+H]⁺.

### Step 6: Preparation of Compound 80

A mixture of CH₃NH₂ (25 mg, 0.80 mmol, 4.0 eq.), compound 80-5 (200 mg, 0.2 mmol, 1.0 eq.), DIEA (2 mL) in DCM (10 mL) was stirred overnight at RT. The mixture was concentrated under vacuum. The residual was purified by prep-HPLC to give the desired product compound 80 (63 mg, 18% yield) as a yellow oil.

¹H NMR (400 MHz, CDCl₃) δ: 0.77-0.98 (m, 12H), 1.14-1.41 (m, 61H), 1.42-1.57 (m, 6H), 1.58-1.72 (m, 8H), 2.13-2.26 (m, 3H), 2.27-2.38 (m, 3H), 2.39-2.56 (m, 6H), 2.57-2.71 (m, 3H), 3.04-3.20 (m, 3H), 3.21-3.36 (m, 3H), 3.67-3.83 (m, 2H), 3.84-4.02 (m, 2H), 5.69-5.83 (m, 1H). LCMS: Rt: 0.950 min; MS m/z (ESI): 972.7 [M+H]⁺.

The following compound was prepared in analogous fashion as Compound 80, using corresponding starting material.

| Compound | Characterization |
|---|---|
| 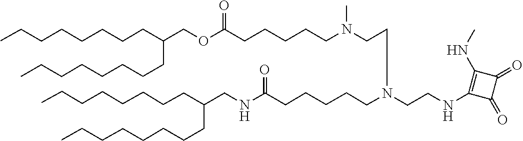 [SMILES: CCCCCCCCC(CCCCCCCC)CNC(=O)CCCCCN(CCNc1c(NC)c(=O)c1=O)CCN(C)CCCCCC(=O)OCC(CCCCCCCC)CCCCCCCC] | ¹H NMR (400 MHz, CDCl₃) δ: 0.86-0.90 (m, 12 H), 1.26-1.38 (m, 65 H), 1.46-1.52 (m, 4H), 1.61-1.67 (m, 6H), 2.18-2.26 (m, 3H), 2.30-2.34 (m, 3H), 2.45-2.52 (m, 4H), 2.59-2.64 (m, 4H), 3.13-3.18 (m, 2H), 3.30-3.31 (m, 3H), 3.76 (s, 2H), 3.96 (d, J = 6.0 Hz, 2H), 5.76 (s, 1H). LCMS: Rt: 0.587 min; MS m/z (ESI): 958.7 [M + H]⁺. |
| Compound 96 | |

### 6.34 Example 34: Preparation of Compound 81

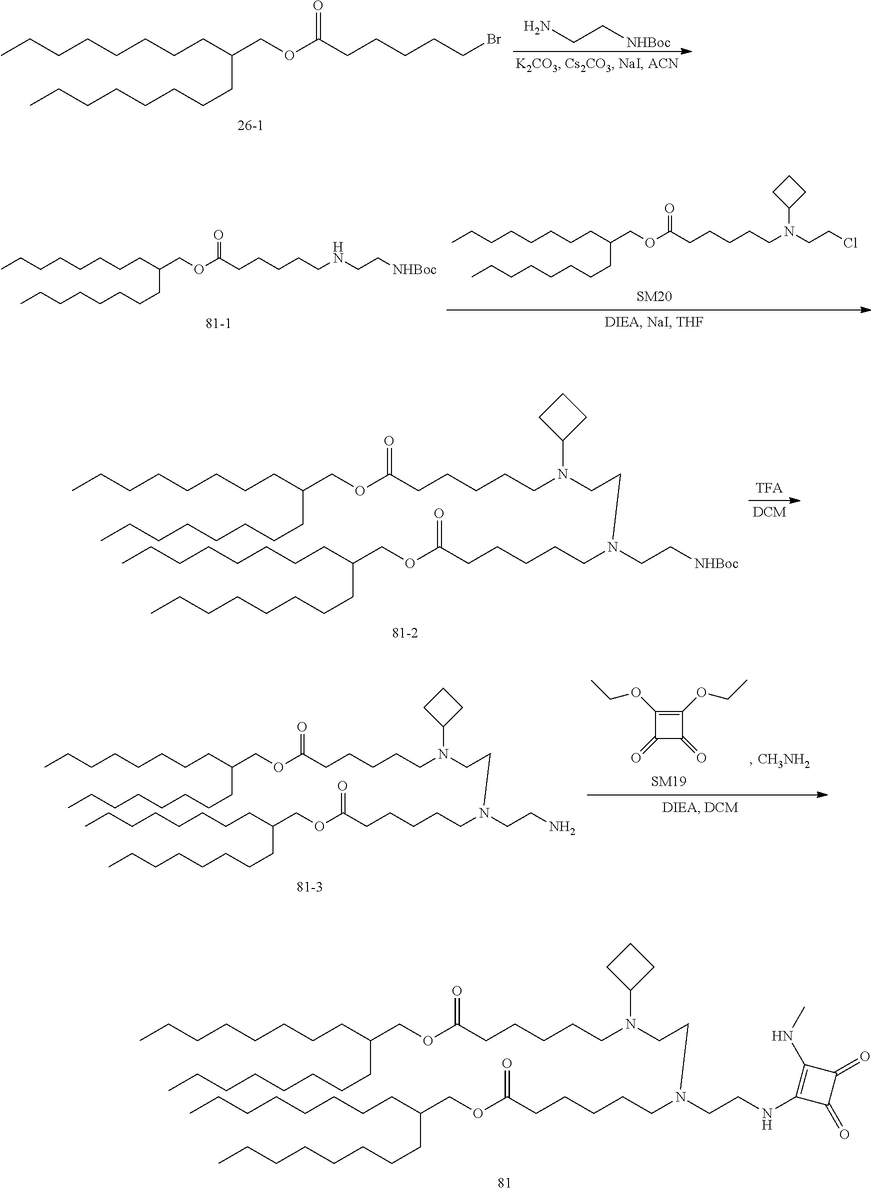

### Step 1: Preparation of Compound 81-1

A mixture of compound 26-1 (2.0 g, 4.47 mmol, 1 eq.), tert-butyl (2-aminoethyl) carbamate (1.1 g, 6.70 mmol, 1.5 eq.), K₂CO₃ (1.8 g, 13.4 mmol, 3.0 eq.), Cs₂CO₃ (440 mg, 1.34 mmol, 0.3 eq.), NaI (200 mg, 1.34 mmol, 0.3 eq.) in ACN (20 mL) was stirred at 90° C. overnight. LCMS showed the target product. The mixture was concentrated and the residue was purified by column chromatography to give product compound 81-1 (1.4 g, 64% yield) as a yellow oil. LCMS: Rt: 0.960 min; MS m/z (ESI): 527.4 [M+H]⁺.

### Step 2: Preparation of Compound 81-2

A mixture of compound 81-1 (500 mg, 0.95 mmol, 1.0 eq.), compound SM20 (570 mg, 1.14 mmol, 1.2 eq.), DIEA (370 mg, 2.85 mmol, 3.0 eq.), NaI (44 mg, 0.29 mmol, 0.3 eq.) in THF (10 mL) was stirred at reflux overnight. LCMS showed the reaction was complete. The mixture was diluted with ethyl acetate and washed with water and brine, dried, concentrated and the residue was purified by column chromatography to give product compound 81-2 (610 mg, 72% yield) as a yellow oil. LCMS: Rt: 2.090 min; MS m/z (ESI): 990.7 [M+H]⁺.

### Step 3: Preparation of Compound 81-3

A mixture of compound 81-2 (300 mg, 0.3 mmol, 1.0 eq.), TFA (345 mg, 3.0 mmol, 10.0 eq.) in DCM (2 mL) was stirred at RT for 4 hours, LCMS showed the reaction was complete. The mixture was concentrated and the residue was used for the next step without further purification. LCMS: Rt: 1.420 min; MS m/z (ESI): 890.7 [M+H]⁺

### Step 4: Preparation of Compound 81

A mixture of compound 81-3 (120 mg, 0.13 mmol, 1.0 eq.), DIEA (170 mg, 1.3 mmol, 10.0 eq.), compound SM19 (46 mg, 0.27 mmol, 2.0 eq.) in DCM (5 mL) was stirred at RT overnight, methylamine (0.67 mmol, 5.0 eq.) was added. LCMS showed the reaction was complete. The residue was purified by Pre-HPLC to give product compound 81 (45 mg, 28% yield) as white solid. ¹H NMR (400 MHz, CDCl₃) δ: 0.86-0.90 (m, 12H), 1.26-1.46 (m, 66H), 1.59-1.64 (m, 6H), 2.21 (s, 6H), 2.29-2.49 (m, 12H), 2.61 (s, 2H), 3.15 (s, 1H), 3.31 (d, J=5.2 Hz, 3H), 3.66-3.75 (m, 2H), 3.94-3.97 (m, 4H). LCMS: Rt: 0.093 min; MS m/z (ESI): 999.7 [M+H]⁺.

The following compounds were prepared in analogous fashion as Compound 81, using corresponding starting material.

| Compound | Characterization |
|---|---|
| 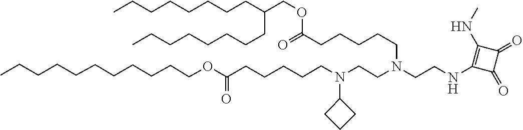 [SMILES: CCCCCCCCCCCOC(=O)CCCCCN(CCN(CCCCCC(=O)OCC(CCCCCCCC)CCCCCCCC)CCNc1c(NC)c(=O)c1=O)C1CCC1] | ¹H NMR (400 MHz, CDCl₃) δ: 0.83-0.93 (m, 9H), 1.14-1.35 (m, 47H), 1.37-1.51 (m, 4H), 1.55-1.67 (m, 8H), 1.75 (s, 14H), 2.2-2.54 (m, 8H), 2.56-2.65(m, 2H), 3.31 (d, J = 4.8 Hz, 3H), 3.73 (s, 1H), 3.91- 3.98 (m, 2H), 4.01-4.10 (m, 2H). LCMS: Rt: 1.340 min; MS m/z (ESI): 901.7 [M + H]⁺. |
| Compound 82 | |
| 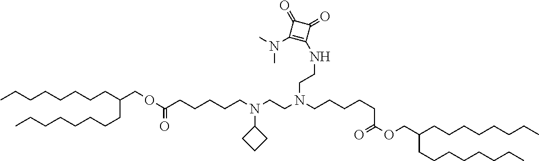 [SMILES: CCCCCCCCC(CCCCCCCC)COC(=O)CCCCCN(CCNc1c(N(C)C)c(=O)c1=O)CCN(CCCCCC(=O)OCC(CCCCCCCC)CCCCCCCC)C1CCC1] | ¹H NMR (400 MHz, CDCl₃) δ: 0.86-0.90 (m, 12H), 1.26 (s, 59H), 1.30-1.45(m, 4H), 1.59-1.66 (m, 6H), 1.82(s, 6H), 1.97- 1.99 (m, 2H), 2.20-2.32 (m, 11H), 2.62- 2.65(m, 2H),3.08-3.11 (m, 1H), 3.24-3.37 (m, 6H), 3.73-3.77 (m, 2H), 3.96-3.97 (m, 4H), 6.24 (s, 1H). LCMS: Rt: 1.800 min; MS m/z (ESI): 1013.8 [M + H]⁺. |
| Compound 85 | |

### 6.35 Example 35: Preparation of Compound 83

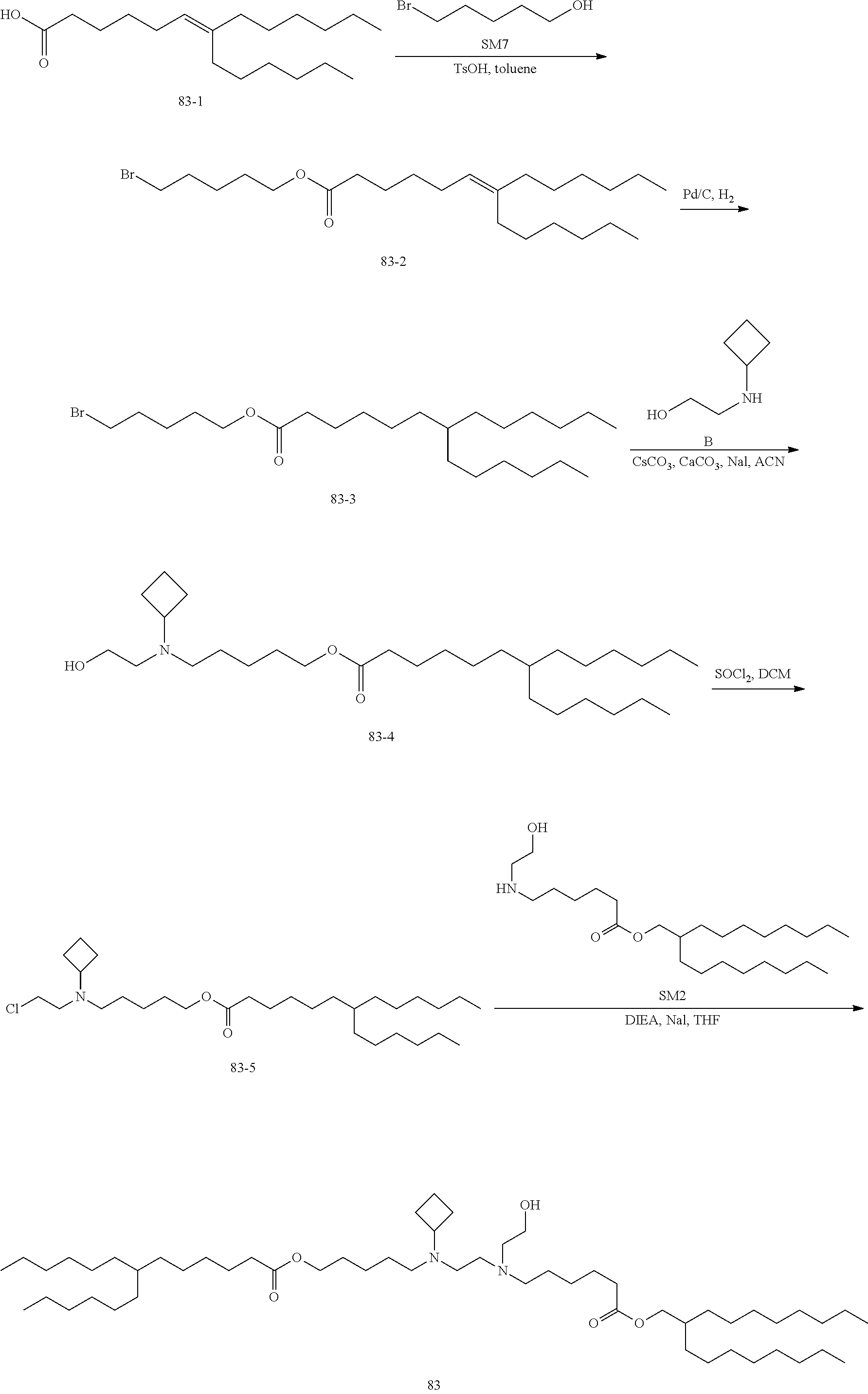

### Step 1: Preparation of Compound 83-2

A mixture of compound 83-1 (1.4 g, 4.73 mmol, 1.0 eq.), compound SM7 (1.2 g, 7.10 mmol, 1.5 eq.), TsOH (20 mg) in toluene (70 mL) was stirred for 2 hours at 180° C. TLC showed the reaction was completed. The mixture was concentrated and purified by column chromatography silica gel (EA:PE=0% to 5%) to give the compound 83-2 (1.0 g, 75% yield) as colorless oil.

### Step 2: Preparation of Compound 83-3

A mixture of compound 83-2 (700 mg, 1.58 mmol, 1.0 eq.) and Pd/C (310 mg, 1.58 mmol, 1.0 eq.) in solution of MeOH (10 mL) and ethyl acetate (10 mL) was stirred for 16 h under H₂ at RT. TLC showed the reaction was completed. The mixture was concentrated to give the desired product compound 83-3 (650 mg, crude) as colorless oil.

### Step 3: Preparation of Compound 83-4

A mixture of compound 83-3 (500 mg, 1.12 mmol, 1.0 eq.), compound B (130 mg, 1.12 mmol, 1.0 eq.) and K₂CO₃ (465 mg, 3.36 mmol, 3.0 eq.), Cs₂CO₃ (110 mg, 0.34 mmol, 0.3 eq.), NaI (15 mg, 0.11 mmol, 0.1 eq.) in ACN (10 mL) was stirred overnight at 85° C. The mixture was concentrated and purified by column chromatography silica gel (MeOH:DCM=0% to 10%) to give the desired product 83-4 (465 mg, 78% yield) as a yellow oil. LCMS: Rt: 0.900 min; MS m/z (ESI): 482.4 [M+H]⁺.

### Step 4: Preparation of Compound 83-5

To a solution of compound 83-4 (185 mg, 0.39 mmol, 1.0 eq.) was dissolved in DCM (10 mL) was added SOCl₂ (230 mg, 2.00 mmol, 5.0 eq.) at RT. The mixture was stirred for 16 hours at 35° C. LCMS show the reaction was completed, the mixture was evaporated under reduced pressure to provide compound 83-5 (185 mg, crude) as a yellow oil. LCMS: Rt: 0.930 min; MS m/z (ESI): 500.4 [M+H]⁺.

### Step 5: Preparation of Compound 83

A mixture of compound SM2 (205 mg, 0.48 mmol, 1.2 eq.), compound 83-5 (200 mg, 0.4 mmol, 1.0 eq.), DIEA (260 mg, 2.0 mmol, 5.0 eq.), NaI (6 mg, 0.04 mmol, 0.1 eq.) in THF (10 mL) was stirred overnight at 75° C. The mixture was concentrated under vacuum. The residual was purified by prep-HPLC to give the desired product compound 83 (63 mg, 18% yield) as a yellow oil.

¹H NMR (400 MHz, CDCl₃) δ: 0.86-0.90 (m, 12H), 1.00-1.41 (m, 59H), 1.42-1.52 (m, 4H), 1.57-1.69 (m, 10H), 1.71-2.12 (m, 4H), 2.22-2.34 (m, 5H), 2.35-2.44 (m, 3H), 2.45-2.56 (m, 4H), 2.57-2.61 (m, 2H), 3.01-3.23 (m, 1H), 3.51-3.55 (m, 2H), 3.90-4.00 (m, 2H), 4.01-4.11 (m, 2H). LCMS: Rt: 1.980 min; MS m/z (ESI): 891.7 [M+H]⁺.

### 6.36 Example 36: Preparation of Compound 84

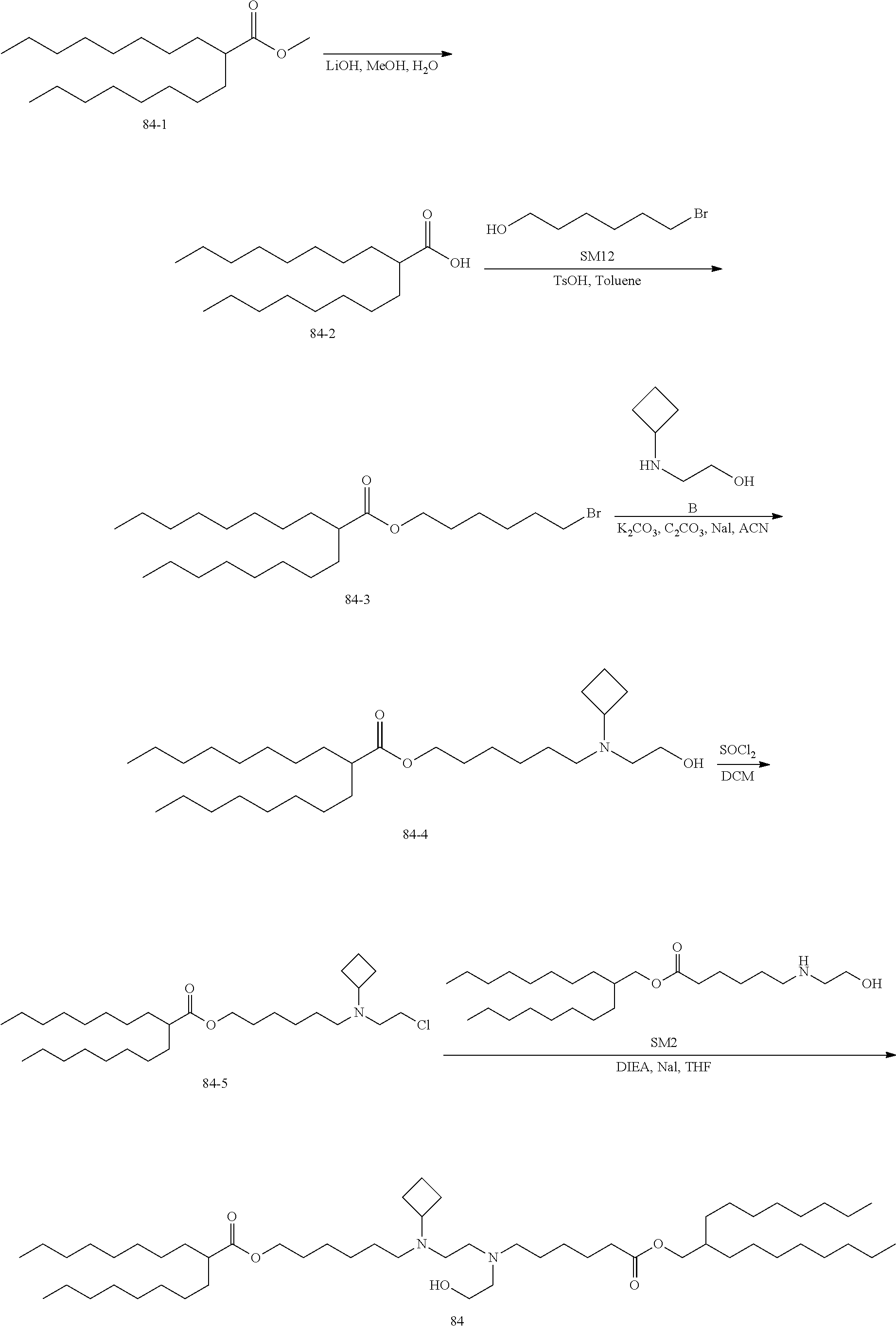

### Step 1: Preparation of Compound 84-2

A mixture of compound 84-1 (1.2 g, 4 mmol, 1.0 eq.) and LiOH H₂O (1.7 g, 40 mmol, 10.0 eq.) with MeOH (30 mL) and H₂O (5 mL) was stirred overnight at 70° C. The mixture was concentrated and extracted over ethyl acetate, dried, concentrated to give the compound 84-2 (853 mg, 74.5% yield) as a yellow oil.

### Step 2: Preparation of Compound 84-3

A mixture of compound 84-2 (853 mg, 3.0 mmol, 1.0 eq.), compound SM12 (648 mg, 3.6 mmol, 1.2 eq.), TsOH (258 mg, 1.5 mmol, 0.5 eq.) in toluene was stirred for 2 hours at 180° C. The mixture was concentrated and purified by column chromatography silica gel (EA:PE=0% to 5%) to give the compound 84-3 (834 mg, 62.1% yield) as colorless oil.

¹H NMR (400 MHz, CDCl₃) δ: 0.79-0.93 (m, 6H), 1.11-1.33 (m, 25H), 1.35-1.51 (m, 6H), 1.59-1.70 (m, 3H), 1.81-2.1 (m, 2H), 2.26-2.35 (m, 1H), 3.36-3.45 (m, 2H), 4.02-4.11 (m, 2H).

### Step 3: Preparation of Compound 84-4

A mixture of compound 84-3 (300 mg, 0.67 mmol, 1.0 eq.), compound B (93 mg, 0.81 mmol, 1.2 eq.), K₂CO₃ (278 mg, 2.01 mmol, 3 eq.), Cs₂CO₃ (7 mg, 0.02 mmol, 0.03 eq.) and sodium iodide (51 mg, 0.34 mmol, 0.5 eq.) in ACN (10 mL) was stirred overnight at 80° C. The mixture was concentrated and purified by column chromatography silica gel (MeOH:DCM=0% to 10%) to give the desired product 84-4 (314 mg, 97.2% yield) as a yellow oil. LCMS: Rt: 0.920 min; MS m/z (ESI): 482.4 [M+H]⁺.

### Step 4: Preparation of Compound 84-5

A mixture of compound 84-4 (314 mg, 0.65 mmol, 1.0 eq.) and SOCl₂ (232 mg, 1.95 mmol, 3.0 eq.) in DCM (10 mL) was stirred overnight at 35° C. The mixture was concentrated to give the desired product 84-5 (343 mg, crude). LCMS: Rt: 1.080 min; MS m/z (ESI): 500.3 [M+H]⁺.

### Step 5: Preparation of Compound 84

A mixture of compound 84-5 (152 mg, 0.3 mmol, 1.0 eq.), compound SM2 (130 mg, 0.3 mmol, 1.0 eq.), sodium iodide (23 mg, 0.15 mmol, 0.5 eq.) and DIEA (116 mg, 0.9 mmol, 3.0 eq.) in THF (5 mL) was stirred overnight at 70° C. The mixture was concentrated under vacuum. The residual was purified by prep-HPLC to give the desired product 84 (54 mg, 19.9% yield) as brown oil. ¹H NMR (400 MHz, CDCl₃) δ: 0.80-0.94 (m, 12H), 1.00-1.34 (m, 59H), 1.36-1.50 (m, 6H), 1.51-1.70 (m, 9H), 1.78-2.05 (m, 5H), 2.26-2.33 (m, 3H), 2.36-2.45 (m, 3H), 2.46-2.55 (m, 4H), 2.57-2.63 (m, 2H), 3.01-3.14 (m, 1H), 3.49-3.59 (m, 2H), 3.93-3.99 (m, 2H), 4.01-4.10 (m, 2H). LCMS: Rt: 2.010 min; MS m/z (ESI): 891.7 [M+H]⁺.

The following compounds were prepared in analogous fashion as Compound 84, using corresponding starting material.

| Compound | Characterization |
|---|---|
| 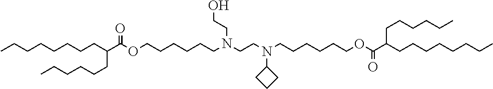 [SMILES: CCCCCCCCC(CCCCCC)C(=O)OCCCCCCN(CCO)CCN(CCCCCCOC(=O)C(CCCCCC)CCCCCCCC)C1CCC1] Compound 69 | ¹H NMR (400 MHz, CDCl₃): 0.85-0.90 (m, 12H), 1.26 (s, 45H), 1.39-1.55 (m, 12H), 1.56-1.71 (m, 10H), 1.76-2.25 (m, 4H), 2.26-2.41 (m, 2H), 2.42-2.82 (m, 8H), 2.85-3.16 (m, 3H), 3.49-3.76 (m, 2H), 4.03-4.07 (m, 4H). LCMS: Rt: 1.380 min; MS m/z (ESI): 835.7 [M + H]⁺. |

### 6.37 Example 37: Preparation of Compound 86

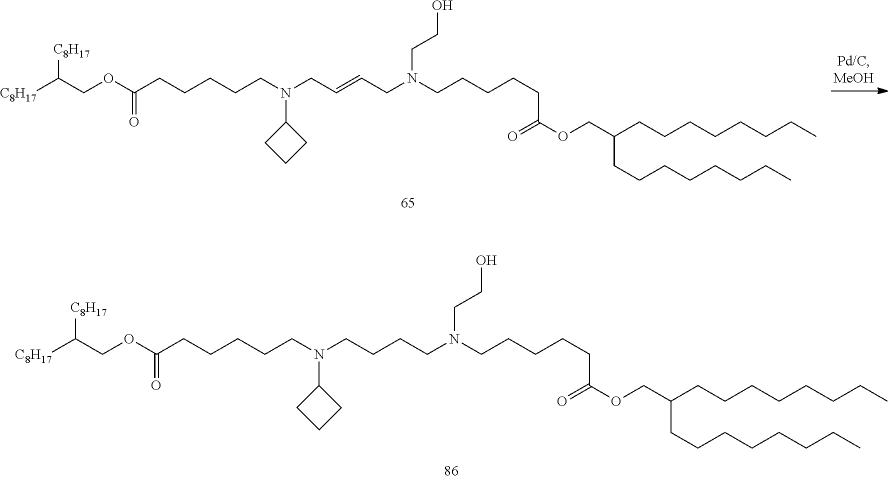

To a solution of compound 65 (0.19 g, 0.21 mmol, 1.0 eq.) in MeOH (5.0 mL) was added Pd/C (30 mg) was stirred for 16 hours at RT under H₂. LCMS showed the reaction was completed, concentrated and purified by pre-HPLC to get the compound 86 (60 mg, 31% yield) as a yellow oil.

¹H NMR (400 MHz, CDCl₃) δ: 0.86-0.90 (m, 12H), 1.26-1.39 (m, 60H), 1.43-1.62 (m, 9H), 1.61-1.67 (m, 8H), 1.77-2.00 (m, 4H), 2.28-2.47 (m, 12H), 2.56-2.58 (m, 2H), 2.95-3.10 (m, 1H), 3.51-3.54 (m, 2H), 3.96 (d, J=6.0 Hz, 4H). LCMS: Rt: 1.490 min; MS m/z (ESI): 919.9 [M+H]⁺.

### 6.38 Example 38: Preparation of Compound 87

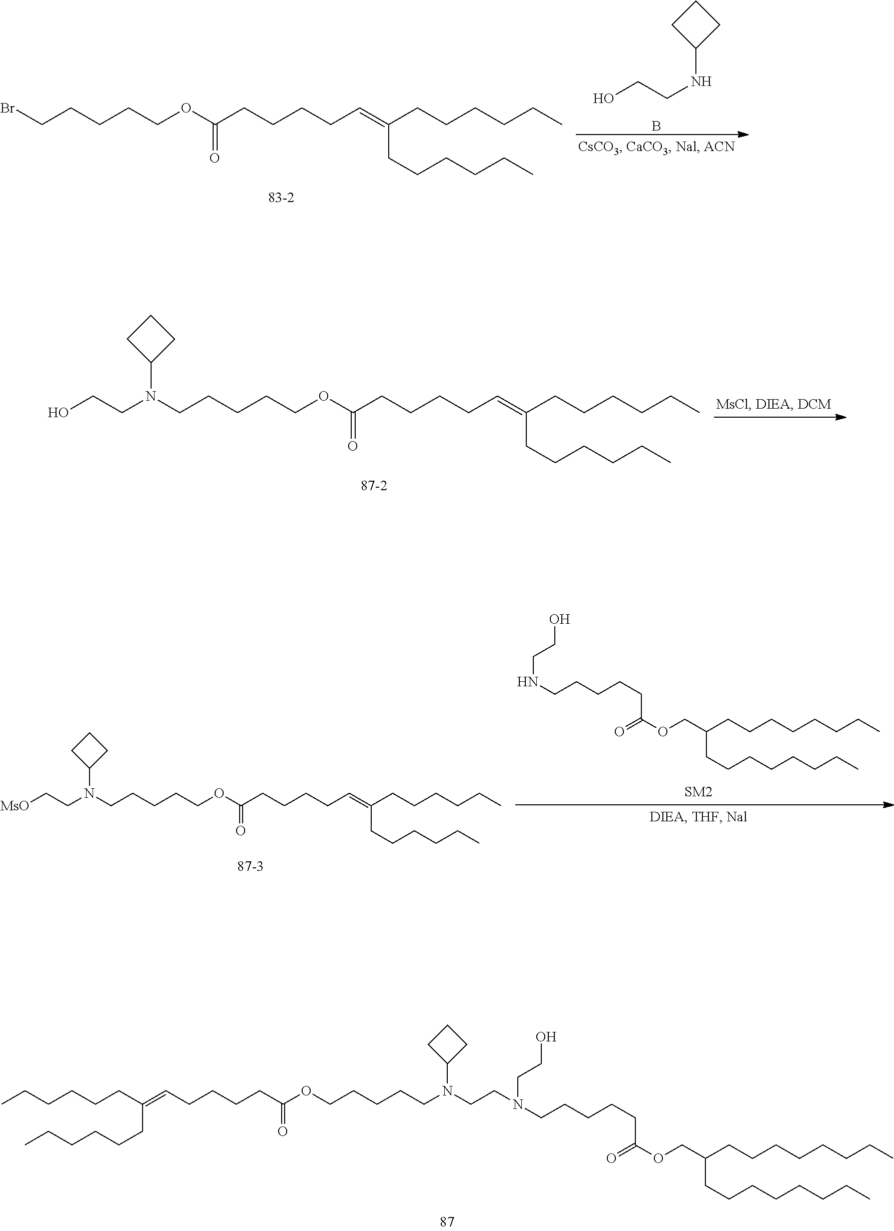

### Step 1: Preparation of Compound 87-2

To a solution of compound 83-2 (500 mg, 1.12 mmol, 1.0 eq.) and compound B (130 mg, 1.12 mmol, 1.0 eq.) was dissolved in ACN (10 mL) was added Cs₂CO₃ (110 mg, 0.34 mmol, 0.3 eq.), K₂CO₃ (465 mg, 3.36 mmol, 3.0 eq.) and NaI (15 mg, 0.11 mmol, 0.1 eq.) at RT. The mixture was stirred for 16 hours at 85° C. LCMS show the reaction was completed, the mixture was evaporated under reduced pressure and purified by FCC (DCM/MeOH=1/0-20/1) to provide compound 87-2 (470 mg, 85% yield) as a yellow oil. LCMS: Rt: 0.930 min; MS m/z (ESI): 480.4 [M+H]⁺.

### Step 2: Preparation of Compound 87-3

To a mixture of compound 87-2 (2.4 g, 5.5 mmol, 1.0 eq.) and MsCl (55 mg, 0.46 mmol, 1.0 eq.) in anhydrous DCM (5 mL) was added DIEA (90 mg, 0.70 mmol, 1.5 eq.) slowly at 0° C. After the addition, the mixture was stirred at RT for 2 hours, TLC showed the reaction was complete. The mixture was washed with water and concentrated. The residue (210 mg) was used for the next step without further purification.

### Step 3: Preparation of Compound 87

To a solution of compound 87-3 (150 mg, 0.27 mmol, 1.0 eq.) and compound SM2 (115 mg, 0.27 mmol, 1.0 eq.) in THF (5 mL) was added DIEA (188 mg, 1.35 mmol, 5.0 eq.) and NaI (5 mg, 0.03 mmol, 0.1 eq.) at 0° C. The mixture was stirred for 16 hours at 75° C. LCMS show the reaction was completed, the mixture was evaporated under reduced pressured purified with pre-HPLC to provide compound 87 (56 mg, 23% yield) as colorless oil.

¹H NMR (400 MHz, CDCl₃) δ: 0.86-0.90 (m, 12H), 1.26-1.39 (m, 49H), 1.58-1.68 (m, 14H), 1.98-2.22 (m, 10H), 2.23-2.34 (m, 5H), 2.38-2.72 (m, 9H), 2.81-3.17 (m, 2H), 3.45-3.65 (m, 2H), 3.95-4.00 (m, 2H), 4.01-4.05 (m, 2H), 5.02-5.12 (m, 1H). LCMS: Rt: 1.680 min; MS m/z (ESI): 889.7 [M+H]⁺.

### 6.39 Example 39: Preparation of Compound 88

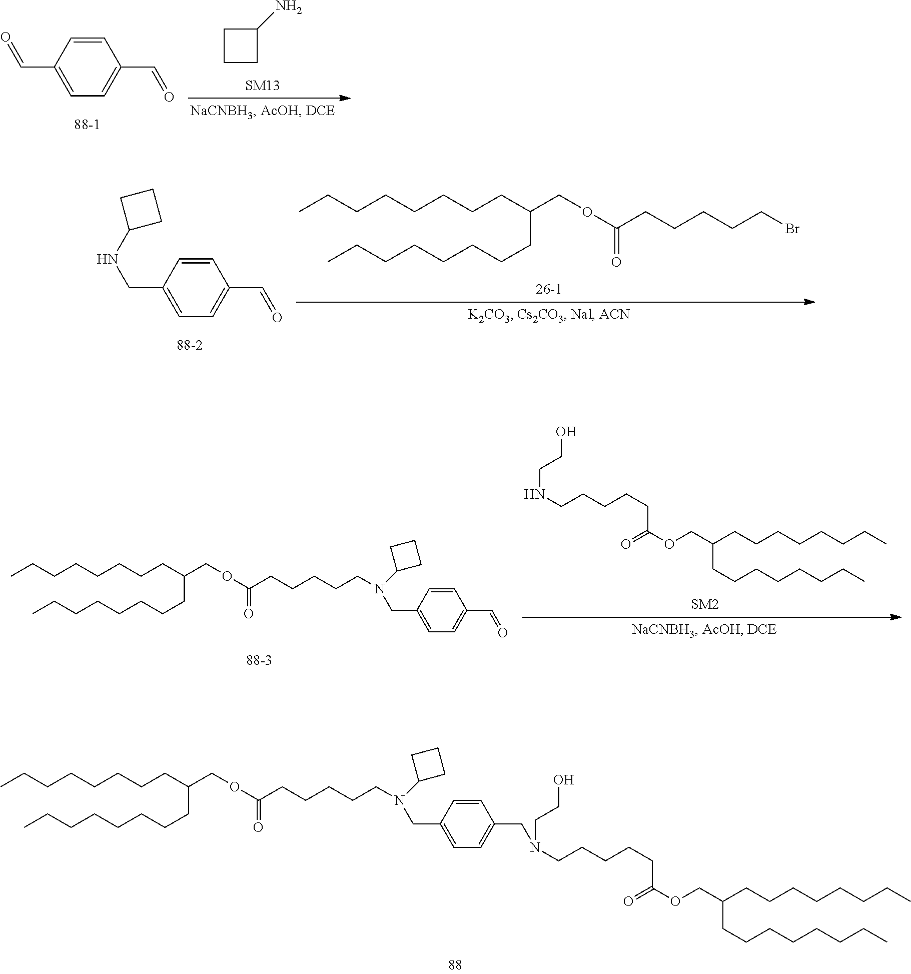

### Step 1: Preparation of Compound 88-2

To a solution of compound 88-1 (1.34 g, 10.0 mmol, 1.0 eq.) in DCE (20.0 mL) was added compound SM13 (0.68 g, 10.0 mmol, 1.0 eq.) and AcOH (0.7 g, 10.0 mmol, 1.0 eq.) was stirred for 2 hours at RT, then NaCNBH₃ (1.02 g, 15.0 mmol, 1.5 eq.) was added and stirred for 16 hours at RT. LCMS showed the reaction was completed, H2O was added, exacted with DCM, concentrated and purified by FCC (DCM/MeOH=20/1) to get the compound 88-2 (0.4 g, 21% yield) as a yellow oil. LCMS: Rt: 0.720 min; MS m/z (ESI): 190.2 [M+H]

### Step 2: Preparation of Compound 88-3

To a solution of compound 88-2 (0.19 g, 1.0 mmol, 1.0 eq.) and compound 26-1 (446.0 mg, 1.0 mmol, 1.0 eq.) in ACN (10.0 mL) was added K₂CO₃ (414 mg, 3.0 mmol, 3.0 eq.), Cs₂CO₃ (97.5 mg, 0.3 mmol, 0.3 eq.) and NaI (14.6 mg, 0.1 mmol, 0.1 eq.) at RT. The mixture was stirred for 16 hours at 85° C. LCMS show the reaction was completed, the mixture was evaporated under reduced pressured purified with FCC (DCM/MeOH=30/1) to provide compound 88-3 (0.26 g, 46% yield) as a yellow oil. LCMS: Rt: 0.990 min; MS m/z (ESI): 556.4 [M+H]⁺.

### Step 3: Preparation of Compound 88

To a solution of compound 88-3 (0.2 g, 0.36 mmol, 1.0 eq.) in DCE (10.0 mL) was added compound SM2 (0.18 g, 0.43 mmol, 1.2 eq.) and AcOH (3 drops) was stirred for 2 hours at RT, then NaBH(OAc)₃ (0.114 g, 0.54 mmol, 1.5 eq.) was added and stirred for 16 hours at RT. LCMS showed the reaction was completed, concentrated and purified by pre-HPLC to get the compound 88 (70 mg, 20% yield) as a yellow oil. ¹H NMR (400 MHz, CDCl₃) δ: 0.86-0.90 (m, 12H), 1.26-1.39 (m, 60H), 1.43-1.62 (m, 13H), 1.78-2.00 (m, 4H), 2.25-2.30 (m, 5H), 2.45-2.62 (m, 5H), 3.10 (s, 1H), 3.49-3.59 (m, 6H), 3.96 (d, J=5.2 Hz, 4H), 7.16-7.22 (m, 4H). LCMS: Rt: 1.350 min; MS m/z (ESI): 967.7 [M+H]⁺.

### 6.40 Example 40: Preparation of Compound 90

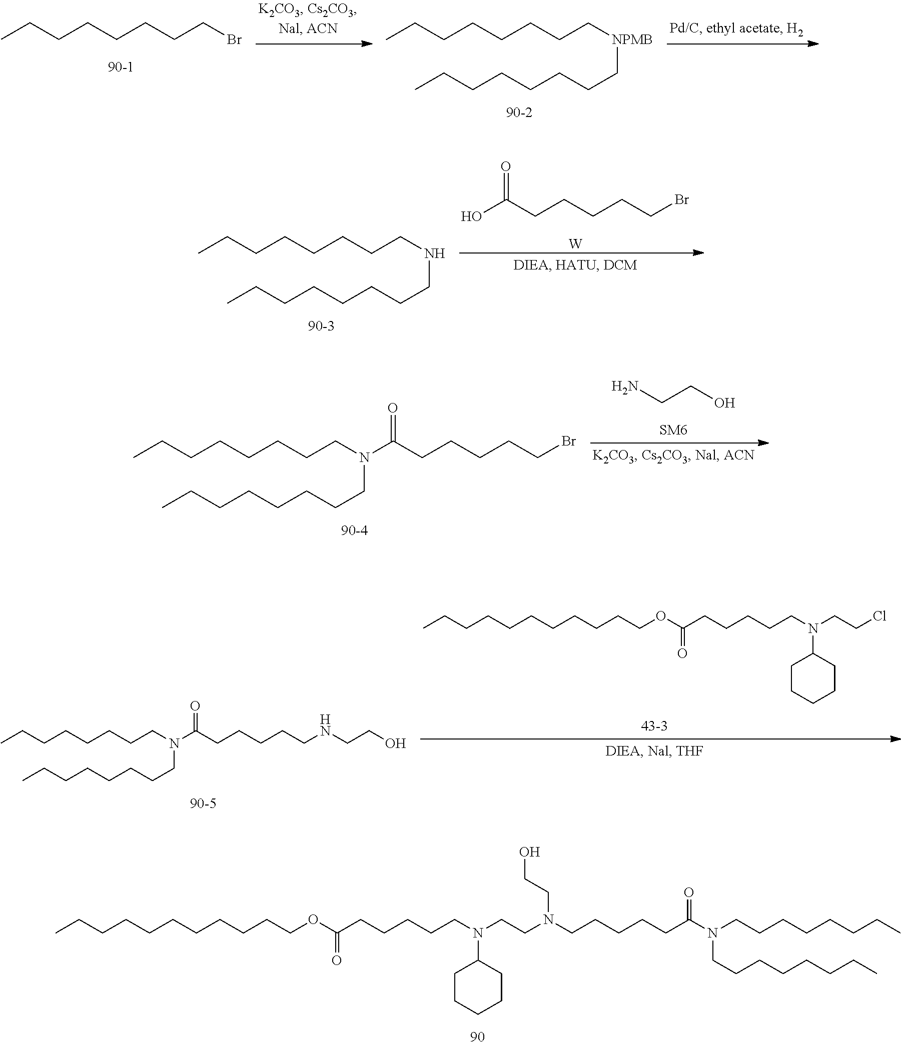

### Step 1: Preparation of Compound 90-2

A mixture of 90-1 (5 g, 25.9 mmol, 3.0 eq.), PMBNH₂ (1.2 g, 8.6 mmol, 1.0 eq.) and K₂CO₃ (3.6 g, 25.9 mmol, 3.0 eq.), Cs₂CO₃ (100 mg, 0.3 mmol, 0.03 eq.) and NaI (0.6 g, 4.3 mmol, 0.5 eq.) in ACN (40 mL) was stirred overnight at 80° C. The mixture was concentrated and purified by column chromatography silica gel (MeOH:DCM=0% to 10%) to give the desired product 90-2 (2.5 g, crude) as a yellow oil. LCMS: Rt: 0.810 min; MS m/z (ESI): 362.3 [M+H]⁺.

### Step 2: Preparation of Compound 90-3

A mixture of compound 90-2 (2.5 g, 6.9 mmol, 1.0 eq.) and Pd/C (10%, 1 g) in solution of MeOH (100 mL) and ethyl acetate (10 mL) was stirred for 5 days at 50° C. The mixture was concentrated to give the desired product 90-3 (1.48 g, crude) as yellow semi-solid. LCMS: Rt: 0.780 min; MS m/z (ESI): 242.3 [M+H]⁺.

### Step 3: Preparation of Compound 90-4

A mixture of compound 90-3 (242 mg, 1 mmol, 1.0 eq.), compound W (214 mg, 1.1 mmol, 1.1 eq.), DIEA (194 mg, 3 mmol, 3.0 eq.) and HATU (418 mg, 1.1 mmol, 1.1 eq.) in DCM (5 mL) was stirred for an hour at RT. The mixture was diluted with water, extracted over ethyl acetate, washed with brine, dried, concentrated and purified over column chromatography silica gel (EA:PE=0% to 33%) to give the desired product 90-4 (298 mg, 71.2% yield) as a yellow oil. LCMS: Rt: 2.250 min; MS m/z (ESI): 418.2, 420.2 [M+H]⁺.

### Step 4: Preparation of Compound 90-5

A mixture of compound 90-4 (298 mg, 0.71 mmol, 1.0 eq.), compound SM6 (130 mg, 2.13 mmol, 3.0 eq.) and K₂CO₃ (295 mg, 2.13 mmol, 3.0 eq.), Cs₂CO₃ (7 mg, 0.02 mmol, 0.03 eq.), NaI (53 mg, 0.35 mmol, 0.5 eq.) in ACN (12 mL) was stirred overnight at 80° C. The mixture was concentrated and purified by column chromatography silica gel (MeOH:DCM=0% to 10%) to give the desired product 90-5 (150 mg, 52.8% yield) as a yellow oil. LCMS: Rt: 0.800 min; MS m/z (ESI): 399.3 [M+H]⁺.

### Step 5: Preparation of Compound 90

A mixture of compound 43-3 (170 mg, 0.3 mmol, 1.0 eq.), compound 90-5 (150 mg, 0.4 mmol, 1.3 eq.), DIEA (116 mg, 0.9 mmol, 3.0 eq.), NaI (23 mg, 0.15 mmol, 0.5 eq.) in THF (5 mL) was stirred overnight at 70° C. The mixture was concentrated under vacuum. The residual was purified by prep-HPLC to give the desired product 90 (82 mg, 34.5% yield) as a yellow oil.

¹H NMR (400 MHz, CDCl₃) δ: 0.80-0.94 (m, 9H), 1.00-1.37 (m, 47H), 1.39-1.56 (m, 8H), 1.57-1.69 (m, 7H), 1.71-1.87 (m, 4H), 2.22-2.34 (m, 4H), 2.36-2.71 (m, 10H), 3.13-3.23 (m, 2H), 3.24-3.38 (m, 2H), 3.48-3.60 (m, 2H), 4.00-4.11 (m, 2H). LCMS: Rt: 1.070 min; MS m/z (ESI): 792.6 [M+H]⁺.

The following compounds were prepared in analogous fashion as Compound 90, using corresponding starting material.

| Compound | Characterization |
|---|---|
| 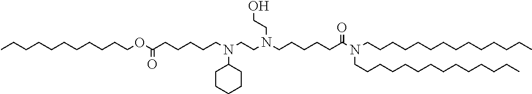 [SMILES: CCCCCCCCCCCCCCN(CCCCCCCCCCCCCC)C(=O)CCCCCN(CCO)CCN(CCCCCC(=O)OCCCCCCCCCCC)C1CCCCC1] Compound 89 | ¹H NMR (400 MHz, CDCl3) δ: 0.82-0.92 (m, 9H), 0.97-1.38 (m, 69H), 1.40-1.54 (m, 8H), 1.55-1.70 (m, 10H), 1.71-1.83 (m, 4H), 2.19-2.34 (m, 4H), 2.37- 2.54 (m, 7H), 2.51-2.62 (m, 2H), 3.13-3.22 (m, 2H), 3.23-3.32 (m, 2H), 3.48-3.57 (m, 2H), 4.00-4.10 (m, 2H). LCMS: Rt: 0.613 min; MS m/z (ESI): 960.7 [M + H]⁺. |

### 6.41 Example 41: Preparation of Compound 100

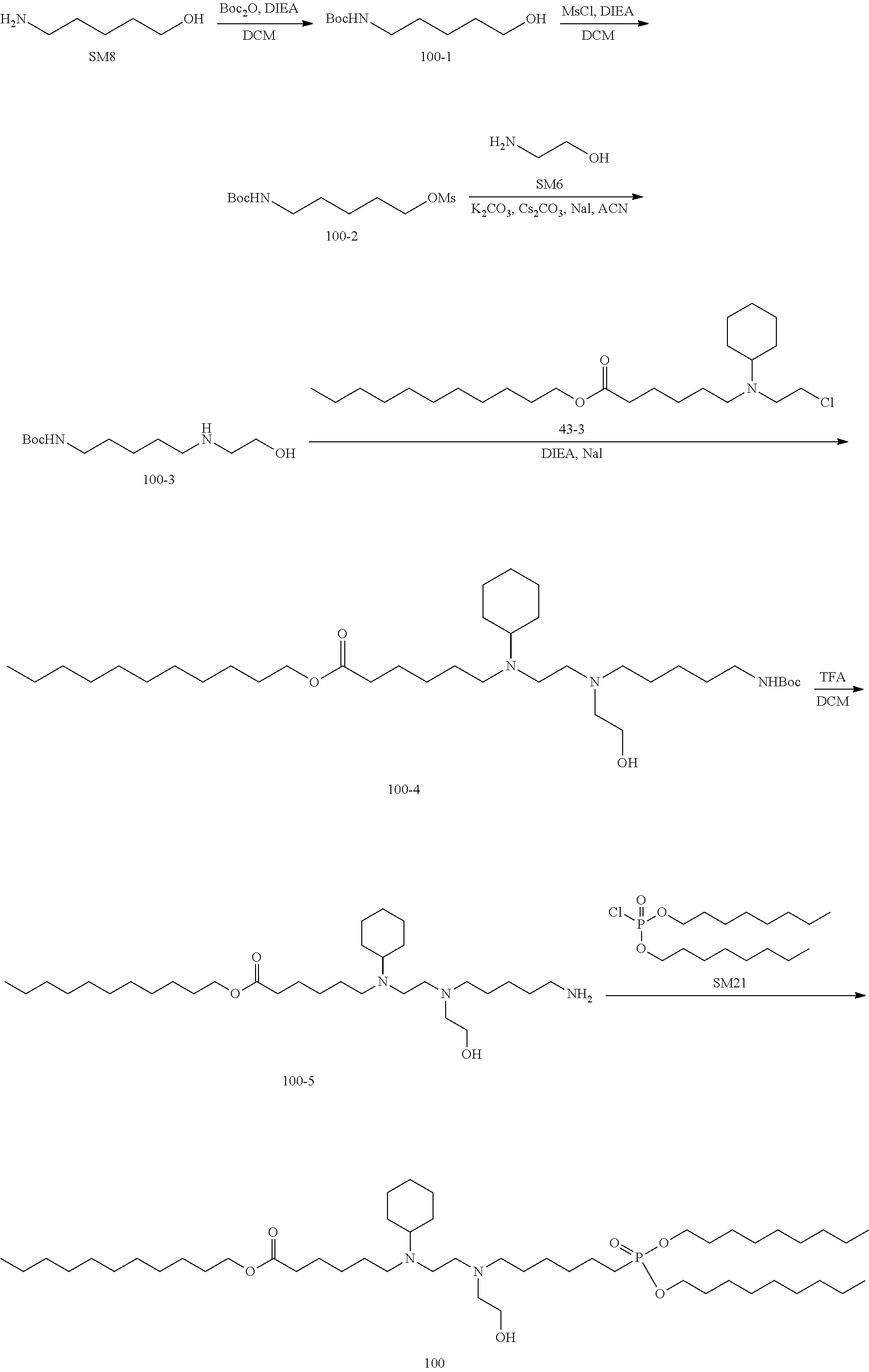

### Step 1: Preparation of Compound 100-1

To a mixture of compound SM8 (2.8 g, 17.1 mmol, 1.0 eq.), DIEA (7.0 g, 54.2 mmol, 2.0 eq.) in DCM (100 mL) was added Boc₂O (7.1 g, 32.6 mmol, 1.2 eq.) at RT. The mixture was stirred for 1 hours at RT, TLC showed the reaction was complete. The mixture was washed with water and brine, dried, concentrated. The residue was purified by column chromatography to give product 100-1 (4.9 g, 86% yield) as colorless oil.

### Step 2: Preparation of Compound 100-2

To a mixture of compound 100-1 (4.8 g, 24.1 mmol, 1.0 eq.), DIEA (6.2 g, 48.2 mmol, 2.0 eq.), DCM (100 mL) was added MsCl (3.3 g, 28.9 mmol, 1.2 eq.) at 0° C. TLC showed the reaction was complete. The mixture was washed with water and brine, dried, concentrated and the residue was purified by column chromatography to give product 100-2 (6.1 g, 90% yield) as a yellow oil.

### Step 3: Preparation of Compound 100-3

A mixture of compound 100-2 (3.0 g, 10.7 mmol, 1.0 eq.), compound SM6 (2.0 g, 32.0 mmol, 3.0 eq.), K₂CO₃ (2.2 g, 16.0 mmol, 1.5 eq.) in ACN (30 mL) was stirred at reflux overnight. LCMS showed the reaction was complete. The mixture was concentrated and the residue was purified by Pre-HPLC to give product 100-3 (2.6 g, 88% yield) as a yellow oil. LCMS: Rt: 0.836 min; MS m/z (ESI): 247.1 [M+H]⁺.

### Step 4: Preparation of Compound 100-4

A mixture of compound 100-3 (500 mg, 2.0 mmol, 1.0 eq.), DIEA (790 mg, 6.1 mmol, 3.0 eq.), compound 43-3 (1.0 g, 2.4 mmol, 1.2 eq.), NaI (90 mg, 0.6 mmol, 0.3 eq.) in THF (20 mL) was stirred at reflux overnight, LCMS showed the reaction was complete. The mixture was concentrated and the residue was purified by column chromatography to give title product (920 mg, 70% yield) as a yellow oil. LCMS: Rt: 0.830 min; MS m/z (ESI): 640.5 [M+H]⁺.

### Step 5: Preparation of Compound 100-5

A mixture of compound 100-4 (920 mg, 1.4 mmol, 1.0 eq.), TFA (2.0 mL) in DCM (5.0 mL) was stirred at reflux overnight, LCMS showed the reaction was complete. The mixture was concentrated and the residue diluted with ethyl acetate and washed with saturated aq. NaHCO₃. The organic layer was concentrated and used for the next step without further purification.

### Step 6: Preparation of Compound 100

To a solution of compound SM21 (crude, 0.05 M, 7.4 mL, 2.0 eq.) in DCM was added compound 100-5 (100 mg, 0.2 mmol, 1.0 eq.) stirred at RT overnight, LCMS showed the target product. The mixture was concentrated and the residue was purified by Pre-HPLC to give 100 (21 mg, 10.8% yield) as a yellow oil.

¹H NMR (400 MHz, CDCl₃) δ: 0.86-0.90 (m, 9H), 1.26-1.30 (m, 46H), 1.41-1.52 (m, 4H), 1.60-1.68 (m, 16H), 1.74-1.81 (m, 4H), 2.27-2.31 (m, 2H), 2.39-2.50 (m, 9H), 2.56-2.60 (m, 2H), 2.86-2.93 (m, 2H), 3.52-3.54 (m, 2H), 3.93-4.00 (m, 4H), 4.03-4.08 (m, 2H). LCMS: Rt: 1.020 min; MS m/z (ESI): 872.7 [M+H]⁺.

### 6.42 Example 42: Preparation of Compound 108

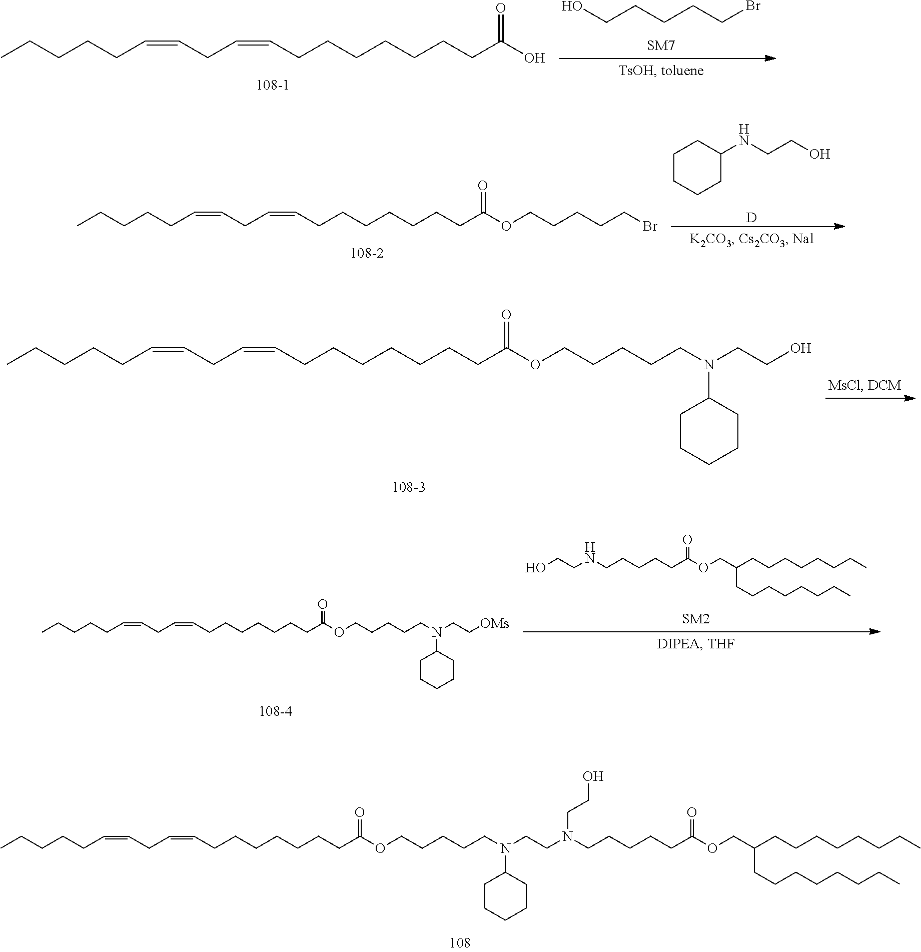

### Step 1: Preparation of Compound 108-2

To a solution of compound 108-1 (758 mg, 4.54 mmol, 1.0 eq.) and compound SM7 (1.4 g, 5.0 mmol, 1.1 eq.) in toluene (40 mL) was added TsOH·H₂O (20 mg). The mixture was stirred under reflux through Dean-Stark trap for 2 hours. The reaction mixture was concentrated and purified by column chromatography on silica gel (PE/EA=50/1) to give the title compound (910 mg, 47% yield) as colorless oil. LCMS: Rt: 0.840 min; MS m/z (ESI): 429.1/431.1[M+H]⁺.

### Step 2: Preparation of Compound 108-3

To a solution of compound 108-2 (910 mg, 2.12 mmol, 1.1 eq.) and compound D (276 mg, 1.93 mmol, 1.0 eq.) in ACN (30 mL) were added K₂CO₃ (799 mg, 5.78 mmol, 3.0 eq.), Cs₂CO₃ (188 mg, 0.58 mmol, 0.3 eq.) and NaI (87 mg, 0.58 mmol, 0.3 eq.). The mixture was stirred at 80° C. for 16 hours. LCMS showed the reaction was complete. The reaction mixture was concentrated and purified by column chromatography on silica gel (DCM/MeOH=20/1) to give the title compound (270 mg, 39% yield) as a yellow oil. LCMS: Rt: 0.880 min; MS m/z (ESI): 492.4 [M+H]⁺.

### Step 3: Preparation of Compound 108-4

To a solution of compound 108-3 (270 mg, 0.55 mmol, 1.0 eq.) and DIPEA (142 mg, 1.10 mmol, 2.0 eq.) in DCM (6 mL) was added MsCl (94 mg, 0.52 mmol, 1.5 eq.). The mixture was stirred at room temperature for 2 hours. LCMS show the reaction was completed. The mixture was poured into water and extracted with DCM. The combined organic layers were washed with brine, dried over Na₂SO₄ and concentrated to give the title compound (313 mg, 100% yield) as a yellow oil. It was used in the next step without further purification. LCMS: Rt: 0.927 min; MS m/z (ESI): 474.2 [M-OMs]⁺.

### Step 4: Preparation of Compound 108

To a solution of compound 108-4 (313 mg, 0.55 mmol, 1.0 eq.) and compound SM2 (235 mg, 0.55 mmol, 1.0 eq.) in THF (10 mL) were added DIPEA (355 mg, 2.75 mmol, 5.0 eq.) and NaI (25 mg, 0.16 mmol, 0.3 eq.). The mixture was stirred at 70° C. for 16 hours LCMS show the reaction was completed. The mixture was concentrated and purified by prep HPLC to give the title compound (115 mg, 23% yield) as a yellow oil. LCMS: Rt: 1.450 min; MS m/z (ESI): 901.7[M+H]⁺.

¹H NMR (400 MHz, CDCl₃) δ: 0.86-0.90 (m, 9H), 1.13-1.36 (m, 53H), 1.43-1.50 (m, 4H), 1.51-1.64 (m, 8H), 1.68-1.79 (m, 4H), 2.02-2.07 (m, 4H), 2.27-2.32 (m, 4H), 2.42-2.54 (m, 8H), 2.62-2.70 (m, 2H), 2.75-2.80 (m, 2H), 3.50-3.56 (m, 2H), 3.96-3.97 (m, 2H), 4.04-4.07 (m, 2H), 5.30-5.43 (m, 4H).

The following compounds were prepared in analogous fashion as Compound 108, using corresponding starting material.

| Compound | Characterization |
|---|---|
| 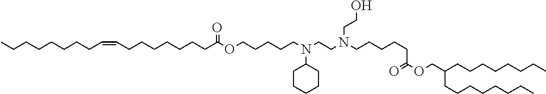 [SMILES: CCCCCCCC/C=C\CCCCCCCC(=O)OCCCCCN(CCN(CCO)CCCCCC(=O)OCC(CCCCCCCC)CCCCCCCC)C1CCCCC1] Compound 109 | ¹H NMR (400 MHz, CDCl3) δ: 0.86-0.90 (m, 9H), 1.10-1.41 (m, 61H), 1.42-1.92 (m, 16H), 1.95-2.12 (m, 5H), 2.22-2.44 (m, 4H), 2.45-2.56 (m, 8H), 3.51-3.53 (m, 1H), 3.95-4.10 (m, 4H), 5.31-5.41 (m, 2H). LCMS: Rt: 1.460 min; MS m/z (ESI): 903.7 [M + H]⁺. |

### 6.43 Example 43: Preparation of Compound 114

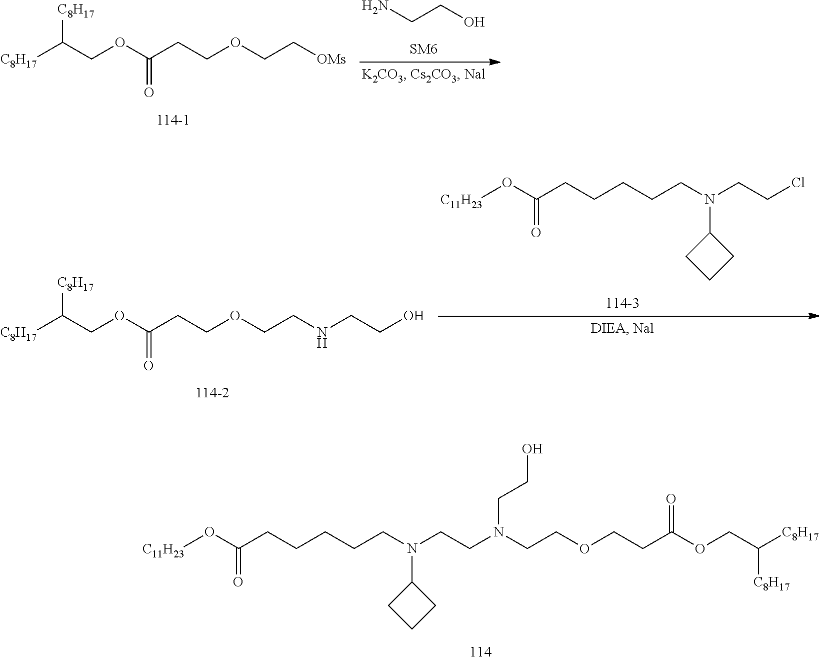

### Step 1: Preparation of Compound 114-2

To a solution of compound 114-1 (1.3 g, 2.8 mmol, 1.0 eq.) in ACN (30 mL) was added compound SM6 (350 mg, 5.59 mmol, 2.0 eq.), K₂CO₃ (1.16 g, 8.39 mmol, 3.0 eq.), Cs₂CO₃ (280 mg, 0.84 mmol, 0.3 eq.) and NaI (130 mg, 0.84 mmol, 0.3 eq.). The reaction mixture was stirred at 80° C. for 10 hours. LCMS showed the reaction was complete. Removal of solvent, FCC to get the compound 114-2 (600 mg, 50%). LCMS: Rt: 0.880 min; MS m/z (ESI): 430.3 [M+H]⁺.

### Step 2: Preparation of Compound 114

To a mixture of compound 114-3 (180 mg, 0.42 mmol, 1.0 eq.), DIEA (150 mg, 1.05 mmol, 2.5 eq.) in THF (10 mL) was added compound 114-2 (150 mg, 0.35 mmol, 0.8 eq.), NaI (50 mg). The reaction mixture was stirred at 70° C. for 10 hours. LCMS showed the reaction was complete. After removal of solvent, the residue was purified by pre-HPLC to give the title compound (30 mg, 11% yield) as a yellow oil. LCMS: Rt: 0.600 min; MS m/z (ESI): 795.5 [M+H]⁺.

¹H NMR (400 MHz, CDCl₃): 0.87 (t, J=8 Hz, 9H), 1.26-1.99 (m, 60H), 2.27-2.31 (m, 2H), 2.39-2.43 (m, 4H), 2.56-2.76 (m, 8H), 3.05-3.09 (m, 1H), 3.50-3.55 (m, 4H), 3.68-3.71 (m, 2H), 3.98-4.07 (m, 4H).

The following compounds were prepared in analogous fashion as Compound 114, using corresponding starting material.

| Compound | Characterization |
|---|---|
| 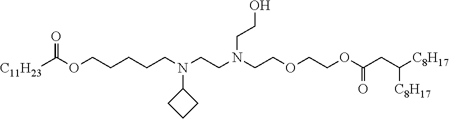 | ¹H NMR (400 MHz, CDCl₃) δ: 0.87 (t, J = 8 Hz, 9H), 1.26-1.99 (m, 59H), 2.25-2.43 (m, 9H), 2.61-2.78 (m, 6H), 3.05-3.09 (m, 1H), 3.53-3.64 (m, 6H), 4.03-4.07 (m, 2H), 4.19-4.22 (m, 2H). LCMS: Rt: 0.493 min; MS m/z (ESI): 795.5 [M + H]⁺. |
| Compound 97 | |
| 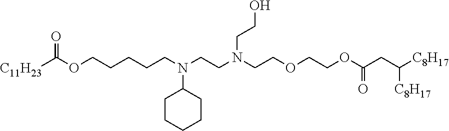 | ¹H NMR (400 MHz, CDCl₃) δ: 0.87 (t, J = 8 Hz, 9H), 1.15-1.80 (m, 63H), 2.25-2.30 (m, 4H), 2.45-2.78 (m, 12H), 3.52-3.65 (m, 6H), 4.04-4.07 (m, 2H), 4.20-4.22 (m, 2H). LCMS: Rt: 0.507 min; MS m/z (ESI): 823.5 [M + H]⁺. |
| Compound 98 | |
| 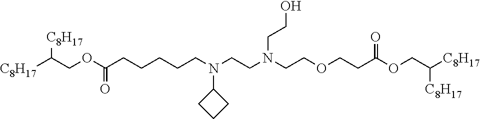 | ¹H NMR (400 MHz, CDCl₃) δ: 0.87 (t, J = 8 Hz, 12H), 1.17-2.01 (m, 70H), 2.28-2.75 (m, 15H), 3.07-3.09 (m, 1H), 3.51-3.55 (m, 4H), 3.68-3.72 (m, 2H), 3.96-4.00 (m, 4H). LCMS: Rt: 0.587 min; MS m/z (ESI): 893.8 [M + H]⁺. |
| Compound 110 | |
| 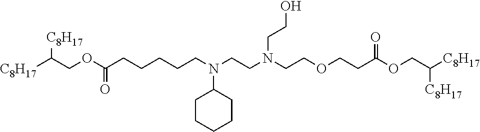 | ¹H NMR (400 MHz, CDCl₃) δ: 0.87 (t, J = 8 Hz, 12H), 1.15-1.79 (m, 74H), 2.28-2.32 (m, 2H), 2.41-2.76 (m, 14H), 3.51-3.53 (m, 4H), 3.69-3.72 (m, 2H), 3.96-4.00 (m, 4H). LCMS: Rt: 0.587 min; MS m/z (ESI): 921.8 [M + H]⁺. |
| Compound 111 | |
| 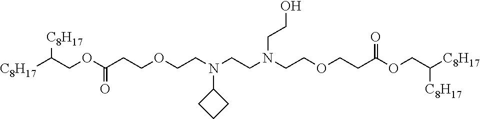 | ¹H NMR (400 MHz, CDCl₃): 0.87 (t, J = 8 Hz, 12H), 1.30-2.02 (m, 64H), 2.49-2.75 (m, 15H), 3.12-3.14 (m, 1H), 3.49-3.54 (m, 6H), 3.67-3.71 (m, 4H), 3.98-4.00 (m, 4H). LCMS: Rt: 0.560 min; MS m/z (ESI): 895.7 [M + H]⁺. |
| Compound 112 | |
| 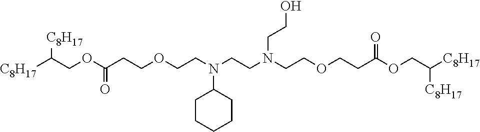 | ¹H NMR (400 MHz, CDCl₃): 0.87 (t, J = 8 Hz, 12H), 1.15-1.79 (m, 68H), 2.55-2.75 (m, 16H), 3.47-3.54 (m, 6H), 3.68-3.72 (m, 4H), 3.98-4.00 (m, 4H). LCMS: Rt: 0.600 min; MS m/z (ESI): 923.8 [M + H]⁺. |
| Compound 113 | |
| 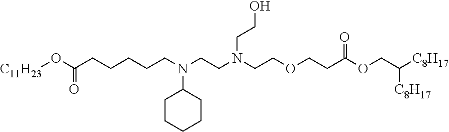 | ¹H NMR (400 MHz, CDCl₃): 0.87 (t, J = 8 Hz, 9H), 1.17-1.80 (m, 63H), 2.29-2.74 (m, 16H), 3.51-3.53 (m, 4H), 3.68-3.70 (m, 2H), 3.98-4.05 (m, 4H). LCMS: Rt: 0.507 min; MS m/z (ESI): 823.6 [M + H]⁺. |
| Compound 115 | |
| 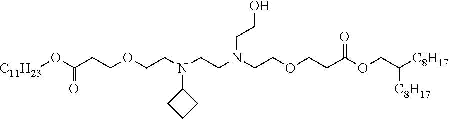 | ¹H NMR (400 MHz, CDCl₃): 0.87 (t, J = 8 Hz, 9H), 1.26-1.99 (m, 54H), 2.25-2.75 (m, 14H), 3.12-3.14 (m, 1H), 3.49-3.71 (m, 10H), 3.98-4.09 (m, 4H). LCMS: Rt: 0.600 min; MS m/z (ESI): 797.6 [M + H]⁺. |
| Compound 116 | |
| 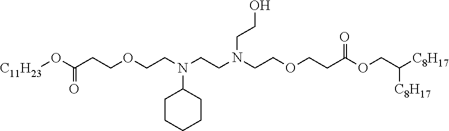 | ¹H NMR (400 MHz, CDCl₃): 0.87 (t, J = 8 Hz, 9H), 1.17-1.79 (m, 58H), 2.54-2.75 (m, 15H), 3.47-3.54 (m, 6H), 3.68-3.72 (m, 4H), 3.98-4.09 (m, 4H). LCMS: Rt: 0.520 min; MS m/z (ESI): 825.7 [M + H]⁺. |
| Compound 117 | |

### 6.44 Example 44: Preparation of Compound 118

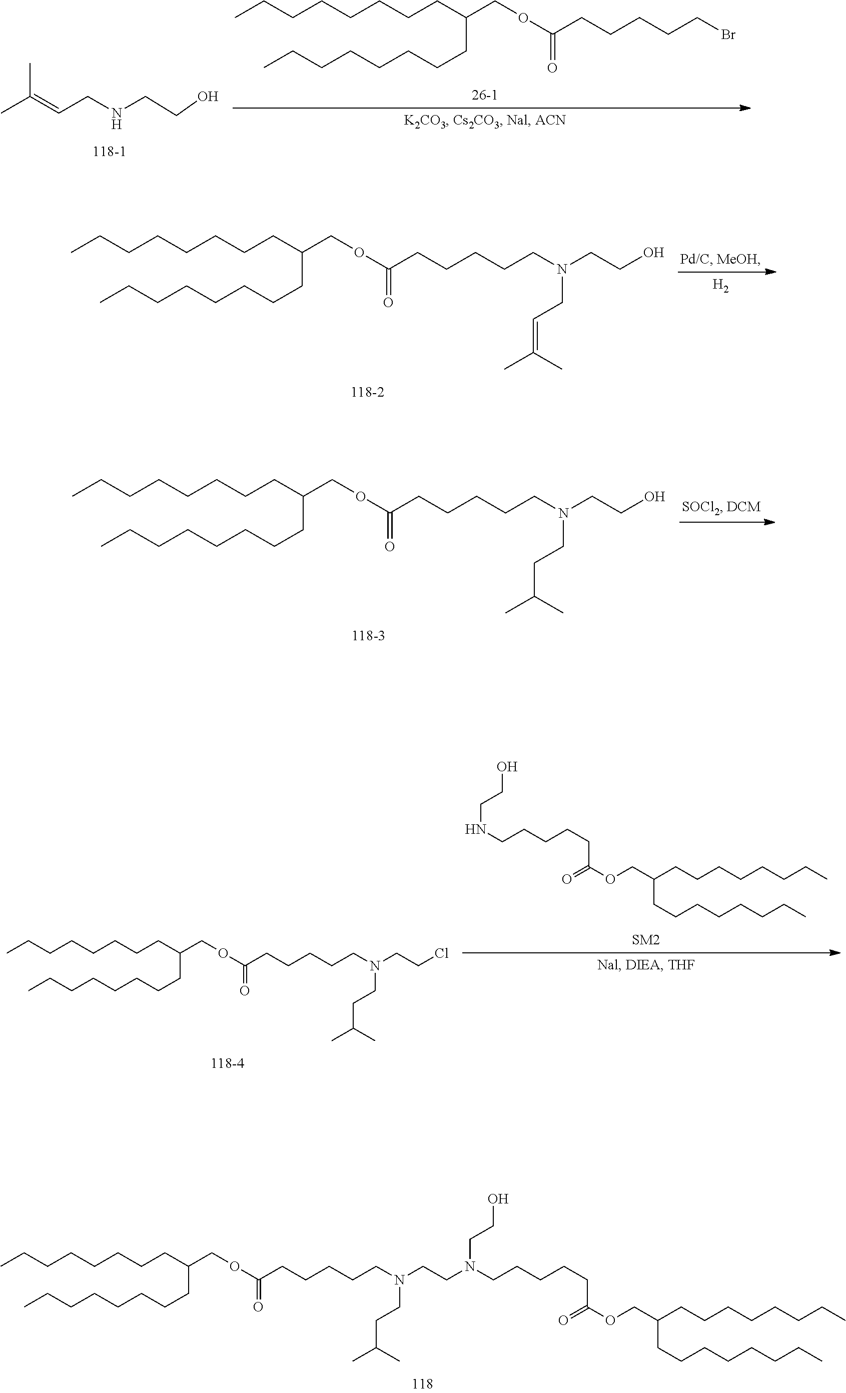

### Step 1: Preparation of Compound 118-2

A mixture of compound 26-1 (200 mg, 0.45 mmol, 1.0 eq.), compound 118-1 (64 mg, 0.50 mmol, 1.1 eq.), K₂CO₃ (186 mg, 1.35 mmol, 3.0 eq.), Cs₂CO₃ (3 mg, 0.01 mmol, 0.03 eq.), NaI (34 mg, 0.23 mmol, 0.5 eq.) in ACN (5 mL) was stirred overnight at 90° C. The mixture was concentrated and purified by column chromatography silica gel (MeOH:DCM=0% to 3%) to give the desired product 118-2 (168 mg, crude) as a yellow oil. LCMS: Rt: 0.890 min; MS m/z (ESI): 496.4 [M+H]⁺.

### Step 2: Preparation of Compound 118-3

A mixture of compound 118-2 (156 mg, 0.3 mmol, 1.0 eq.), Pd/C (15 mg) in MeOH (6 mL) was stirred overnight at 40° C. under hydrogen atmosphere. The mixture filtered, the filtrate was concentrated and purified by column chromatography silica gel (MeOH:DCM=0% to 3%) to give the desired product 118-3 (145 mg, 92.5% yield) as a yellow oil. LCMS: Rt: 1.780 min; MS m/z (ESI): 498.5 [M+H]⁺.

### Step 3: Preparation of Compound 118-4

A mixture of compound 118-3 (148 mg, 0.3 mmol, 1.0 eq.) and SOCl₂ (108 mg, 0.9 mmol, 3.0 eq.) in DCM (5 mL) was stirred overnight at 35° C. The mixture was concentrated to give the desired product 118-4 (167 mg, crude) as a yellow oil. LCMS: Rt: 1.140 min; MS m/z (ESI): 516.4 [M+H]⁺.

### Step 4: Preparation of Compound 118

A mixture of compound 118-4 (167 mg, 0.33 mmol, 1.0 eq.), compound SM2 (170 mg, 0.40 mmol, 1.2 eq.), DIEA (128 mg, 0.99 mmol, 3.0 eq.) and NaI (24 mg, 0.16 mmol, 0.5 eq.) in THF (5 mL) was stirred overnight at 70° C. The mixture was concentrated under vacuum. The residual was purified by prep-HPLC twice to give the desired product 118 (22 mg, 7.5% yield) as brown oil. LCMS: Rt: 2.440 min; MS m/z (ESI): 907.8 [M+H]⁺.

¹H NMR (400 MHz, CDCl₃) δ: 0.81-0.94 (m, 18H), 1.18-1.38 (m, 62H), 1.41-1.56 (m, 5H), 1.58-1.70 (m, 7H), 2.20-2.35 (m, 4H), 2.37-2.64 (m, 12H), 3.45-3.56 (m, 2H), 3.91-4.01 (m, 4H).

### 6.45 Example 45: Preparation of Compound 120

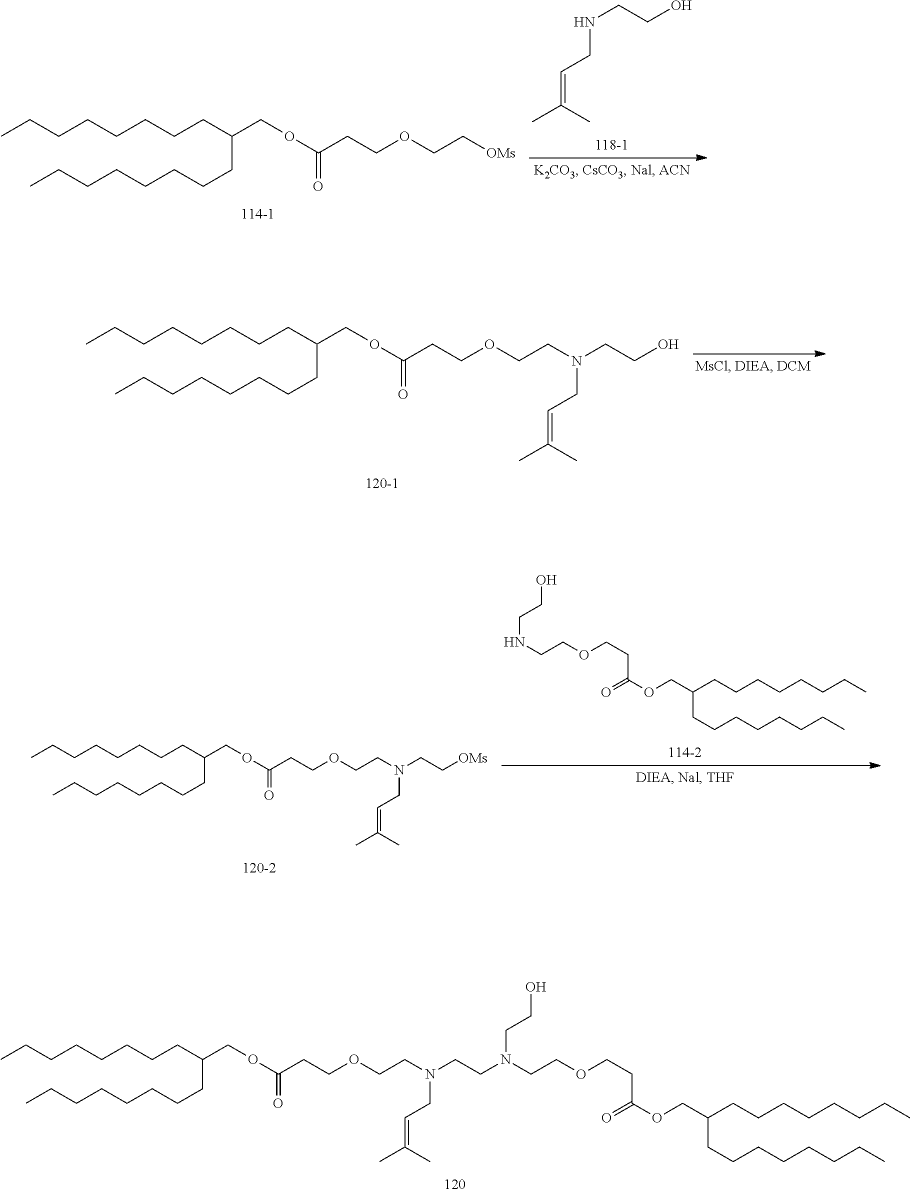

### Step 1: Preparation of Compound 120-1

A mixture of compound 114-1 (300 mg, 0.65 mmol, 1.0 eq.), compound 118-1 (125 mg, 0.97 mmol, 1.5 eq.) and K₂CO₃ (269 mg, 1.94 mmol, 3.0 eq.), Cs₂CO₃ (65 mg, 0.20 mmol, 0.3 eq.), NaI (8 mg, 0.06 mmol, 0.1 eq.) in ACN (10 mL) was stirred overnight at 85° C. The mixture was concentrated and purified by column chromatography silica gel (MeOH:DCM=0% to 10%) to give the desired product 120-1 (150 mg, 46% yield) as a yellow oil. LCMS: Rt: 0.890 min; MS m/z (ESI): 498.4 [M+H]⁺.

### Step 2: Preparation of Compound 120-2

To a solution of compound 120-1 (150 mg, 0.30 mmol, 1.0 eq.) was dissolved in DCM (10 mL) was added DIEA (58 mg, 0.45 mmol, 1.5 eq.) and MsCl (52 mg, 0.45 mmol, 1.5 eq.) at RT. The mixture was stirred for 1 hours at RT. TLC show the reaction was completed, the mixture was evaporated under reduced pressure to provide 120-2 (110 mg, 64% yield) as a yellow oil.

### Step 2: Preparation of Compound 120

A mixture of compound 114-2 (103 mg, 0.24 mmol, 1.2 eq.), compound 120-2 (110 mg, 0.20 mmol, 1.0 eq.), DIEA (77 mg, 0.60 mmol, 3.0 eq.), NaI (8 mg, 0.06 mmol, 0.3 eq.) in THF (10 mL) was stirred overnight at 75° C. The mixture was concentrated under vacuum. The residual was purified by prep-HPLC to give the desired product 120 (15 mg, 8% yield) as a yellow oil. LCMS: Rt: 2.110 min; MS m/z (ESI): 909.8 [M+H]⁺.

¹H NMR (400 MHz, CDCl₃) δ: 0.80-0.95 (m, 12H), 1.16-1.36 (m, 52H), 1.53-1.70 (m, 6H), 1.71-1.85 (m, 5H), 2.49-2.84 (m, 15H), 3.03-3.19 (m, 1H), 3.43-3.64 (m, 6H), 3.77-3.90 (m, 5H), 3.91-4.03 (m, 5H), 5.19-5.33 (m, 1H).

The following compounds were prepared in analogous fashion as Compound 120, using corresponding starting material.

| Compound | Characterization |
|---|---|
| 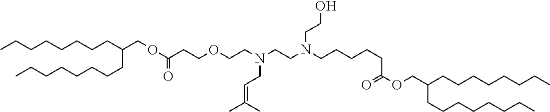 [SMILES: CCCCCCCCC(CCCCCCCC)COC(=O)CCCCCN(CCO)CCN(CC=C(C)C)CCOCCC(=O)OCC(CCCCCCCC)CCCCCCCC] Compound 123 | ¹H NMR (400 MHz, CDCl₃) δ: 0.76-0.93 (m, 12H), 1.16-1.37 (m, 54H), 1.52-1.70 (m, 8H), 1.72-1.85 (m, 4H), 1.91-2.23 (m, 7H), 2.24-2.36 (m, 3H), 2.37-2.64 (m, 7H), 3.03-3.27 (m, 3H),3.42-3.65 (m, 3H), 3.66-3.86 (m, 4H), 3.86-4.03 (m, 4H), 5.18-5.37 (m, 1H). LCMS: Rt: 1.870 min; MS m/z (ESI): 907.8 [M + H]⁺. |

### 6.46 Example 46: Preparation of Compound 127

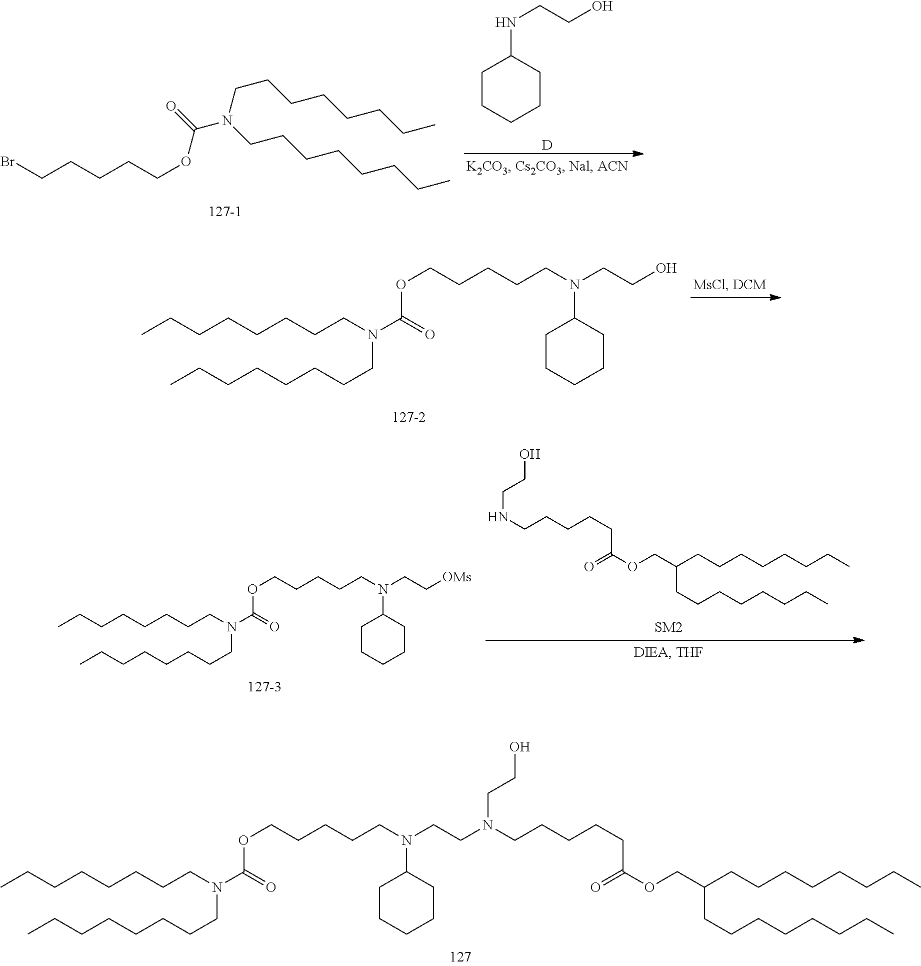

### Step 1: Preparation of Compound 127-2

To a solution of compound 127-1 (300.0 mg, 0.7 mmol, 1.0 eq.) and compound D (97.0 mg, 0.7 mmol, 1.0 eq.) in ACN (15.0 mL) was added K₂CO₃ (276.0 mg, 2.01 mmol, 3.0 eq.), Cs₂CO₃ (65.0 mg, 0.2 mmol, 0.3 eq.) and NaI (10.0 mg, 0.07 mmol, 0.1 eq.) at RT. The mixture was stirred for 16 hours at 85° C. LCMS show the reaction was completed, the mixture was evaporated under reduced pressured purified with FCC (DCM/MeOH=1/0-10/1) to provide 127-2 (0.24 g, crude) as a yellow oil. LCMS: Rt: 0.813 min; MS m/z (ESI): 497.4 [M+H]⁺.

### Step 2: Preparation of Compound 127-3

To a solution of compound 127-2 (0.24 g, 0.48 mmol, 1.0 eq.) in DCM (10.0 mL) and was added DIEA (124.0 mg, 0.96 mmol, 2.0 eq.) and MsCl (67.0 mg, 0.58 mmol, 1.2 eq.) at 0° C. The mixture was stirred for 1 hours. TLC show the reaction was completed, the H₂O was added and extracted with DCM, dried over Na₂SO₄. The mixture was evaporated under reduced pressured to provide 127-3 (0.26 g, crude) as a yellow oil.

### Step 3: Preparation of Compound 127

To a solution of compound 127-3 (240.0 mg, 0.4 mmol, 1.0 eq.) and compound SM2 (180.0 mg, 0.4 mmol, 1.0 eq.) in THF (5.0 mL) was added DIEA (162.0 mg, 1.2 mmol, 3.0 eq.) and NaI (6.0 mg, 0.04 mmol, 0.1 eq.) at 0° C. The mixture was stirred for 16 hours at 70° C. LCMS show the reaction was completed, the mixture was evaporated under reduced pressured purified with pre-HPLC to provide 127 (35.0 mg, 9% yield) as a yellow oil.

¹H NMR (400 MHz, CDCl₃) δ: 0.86-0.90 (m, 12H), 1.14-1.36 (m, 59H), 1.42-1.78 (m, 18H), 2.20-2.60 (m, 12H), 3.17 (s, 4H), 3.53 (s, 2H), 3.96-4.06 (m, 4H). LCMS: Rt: 1.680 min; MS m/z (ESI): 906.9 [M+H]⁺.

### 6.47 Example 47: Preparation of Compound 128

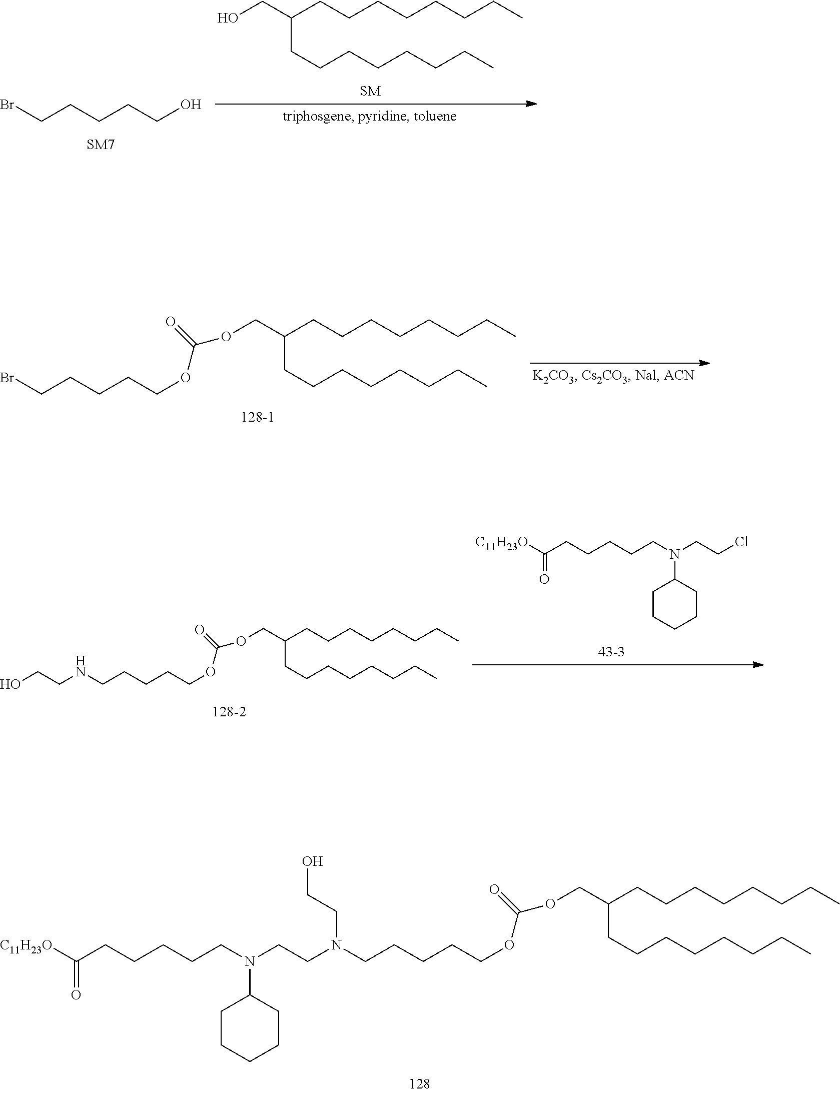

### Step 1: Preparation of Compound 128-1

To a solution of compound SM7 (332.0 mg, 2.0 mmol, 1.0 eq.) was dissolved in toluene (10.0 mL) at 0° C., then pyridine (1.1 g, 16.0 mmol, 8.0 eq.) and triphosgene (1.1 g, 1.2 mmol, 0.6 eq.) was added. The mixture was stirred for 1 hours at RT then compound SM (540.0 mg, 2.0 mmol, 1.0 eq.) was added. The mixture was stirred for 16 hours at RT. TLC show the reaction was completed, the mixture was poured into H₂O, extracted with EA. The mixture was evaporated under reduced pressure and purified by FCC (PE/EA=100/1-10/1) to provide 128-1 (350.0 mg, crude) as a yellow oil.

### Step 2: Preparation of Compound 128-2

To a solution of compound 128-1 (300.0 mg, 0.65 mmol, 1.0 eq.) and ethanol amine (120.0 mg, 2.0 mmol, 3.0 eq.) in ACN (15.0 mL) was added DIEA (419.0 mg, 3.25 mmol, 5.0 eq.) at RT. The mixture was stirred for 16 hours at 70° C. LCMS show the reaction was completed, the mixture was evaporated under reduced pressured purified with FCC (DCM/MeOH=1/0-10/1) to provide 128-2 (0.2 g, crude) as a yellow oil. LCMS: Rt: 0.882 min; MS m/z (ESI): 444.4 [M+H]⁺.

### Step 3: Preparation of Compound 128

To a solution of compound 128-2 (155.0 mg, 0.35 mmol, 1.0 eq.) and compound 43-3 (150.0 mg, 0.35 mmol, 1.0 eq.) in THF (5.0 mL) was added DIEA (135 mg, 1.2 mmol, 5.0 eq.) and NaI (5.0 mg, 0.035 mmol, 0.1 eq.) at 0° C. The mixture was stirred for 16 hours at 70° C. LCMS show the reaction was completed, the mixture was evaporated under reduced pressured purified with pre-HPLC to provide 128 (20.0 mg, 7% yield) as a yellow oil. LCMS: Rt: 1.380 min; MS m/z (ESI): 837.7[M+H]⁺.

¹H NMR (400 MHz, CDCl₃) δ: 0.86-0.90 (m, 9H), 1.26-1.38 (m, 57H), 1.59-1.71 (m, 16H), 2.28-2.32 (m, 2H), 2.52-2.70 (m, 8H), 3.50-3.61 (m, 2H), 4.03-4.14 (m, 6H).

### 6.48 Example 48: Preparation of Compound 133

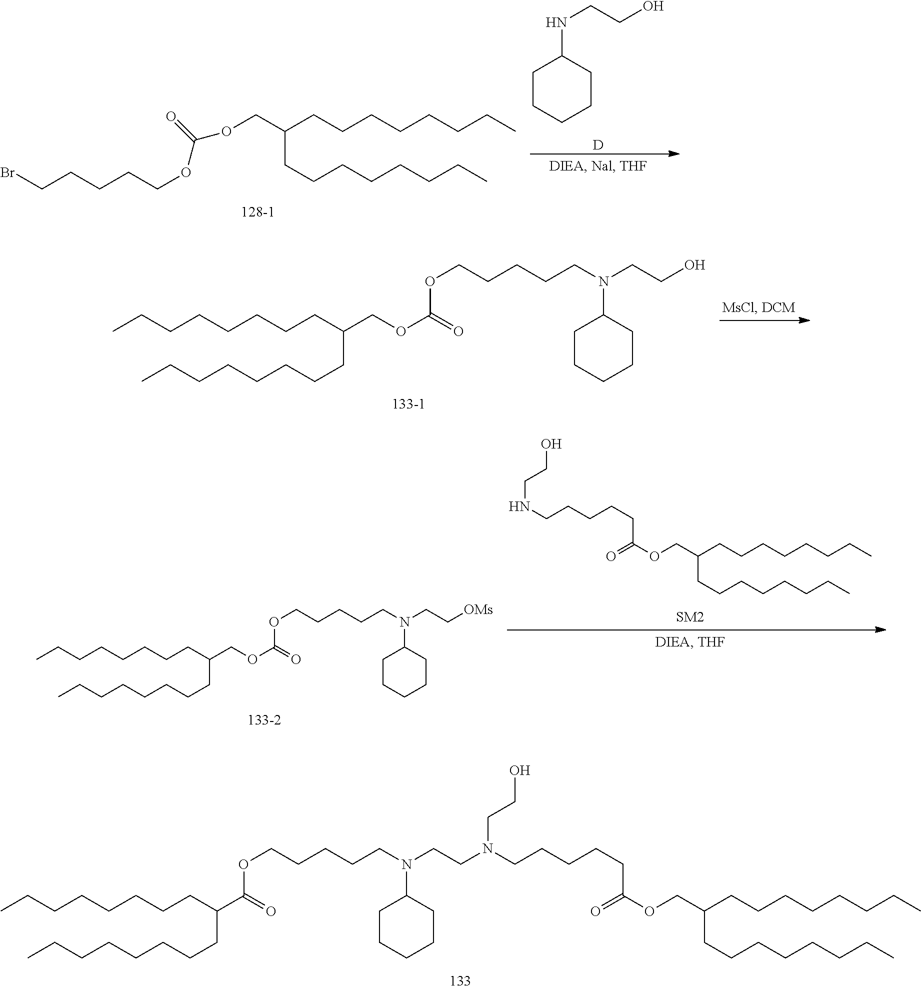

### Step 1: Preparation of Compound 133-1

To a solution of compound 128-1 (500.0 mg, 1.0 mmol, 1.0 eq.) and compound D (158.0 mg, 1.0 mmol, 1.0 eq.) in ACN (15.0 mL) was added DIEA (418.0 mg, 3.0 mmol, 3.0 eq.) and NaI (15.0 mg, 0.1 mmol, 0.1 eq.) at RT. The mixture was stirred for 16 hours at 70° C. LCMS show the reaction was completed, the mixture was evaporated under reduced pressured purified with FCC (DCM/MeOH=1/0-10/1) to provide 133-1 (0.3 g, crude) as a yellow oil. LCMS: Rt: 0.945 min; MS m/z (ESI): 526.5 [M+H]⁺.

### Step 2: Preparation of Compound 133-2

To a solution of compound 133-1 (0.3 g, 0.57 mmol, 1.0 eq.) in DCM (10.0 mL) and was added DIEA (147.0 mg, 1.14 mmol, 2.0 eq.) and MsCl (79.0 mg, 0.68 mmol, 1.2 eq.) at 0° C. The mixture was stirred for 1 hours. TLC show the reaction was completed, the H₂O was added and extracted with DCM, dried over Na₂SO₄. The mixture was evaporated under reduced pressured to provide 133-2 (0.35 g, crude) as a yellow oil.

### Step 3: Preparation of Compound 133

To a solution of compound 133-2 (240.0 mg, 0.4 mmol, 1.0 eq.) and compound SM2 (180.0 mg, 0.4 mmol, 1.0 eq.) in THF (5.0 mL) was added DIEA (162.0 mg, 1.2 mmol, 3.0 eq.) and NaI (6.0 mg, 0.04 mmol, 0.1 eq.) at 0° C. The mixture was stirred for 16 hours at 70° C. LCMS show the reaction was completed, the mixture was evaporated under reduced pressured purified with pre-HPLC to provide 133 (25.0 mg, 7% yield) as a yellow oil. LCMS: Rt: 2.170 min; MS m/z (ESI): 935.8 [M+H]

¹H NMR (400 MHz, CDCl₃) δ: 0.86-0.90 (m, 12H), 1.14-1.36 (m, 68H), 1.60-1.82 (m, 15H), 2.28-2.32 (m, 2H), 2.54-2.64 (m, 9H), 3.58 (s, 2H), 3.98-4.14 (m, 6H).

### 6.49 Example 49: Preparation of Compound 134

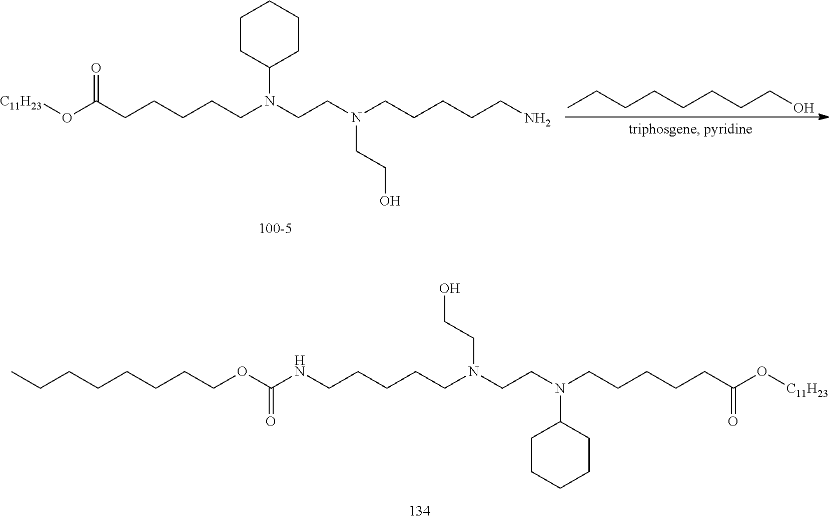

### Step 1: Preparation of Compound 134

To a solution of triphosgene (300 mg, 1.0 mmol, 1.0 eq.) in DCM (20 mL) in was added octanol (395 mg, 3.0 mmol, 3.0 eq.) and pyridine (640 mg, 8.0 mmol, 8.0 eq.) at RT. The mixture was stirred for 1 h. Compound 100-5 (100 mg, 0.20 mmol, 0.2 eq.) was added to the reaction mixture (4 mL). The mixture was stirred overnight, LCMS showed the target product. The mixture was concentrated and the residue was purified by pre-HPLC to give product 134 (16 mg, 4.3% yield) as a yellow oil.

¹H NMR (400 MHz, CDCl₃) δ: 0.86-0.88 (m, 6H), 1.07-1.30 (m, 32H), 1.47-1.59 (m, 26H), 1.78-1.79 (m, 4H), 1.98-2.04 (m, 2H), 2.26-2.58 (m, 6H), 2.10-3.12 (m, 1H), 3.49-3.53 (m, 1H), 4.03-4.07 (m, 2H), 4.66-5.38 (m, 1H). LCMS: Rt: 0.880 min; MS m/z (ESI): 696.6 [M+H]⁺.

### 6.50 Example 50: Preparation of Compound 147

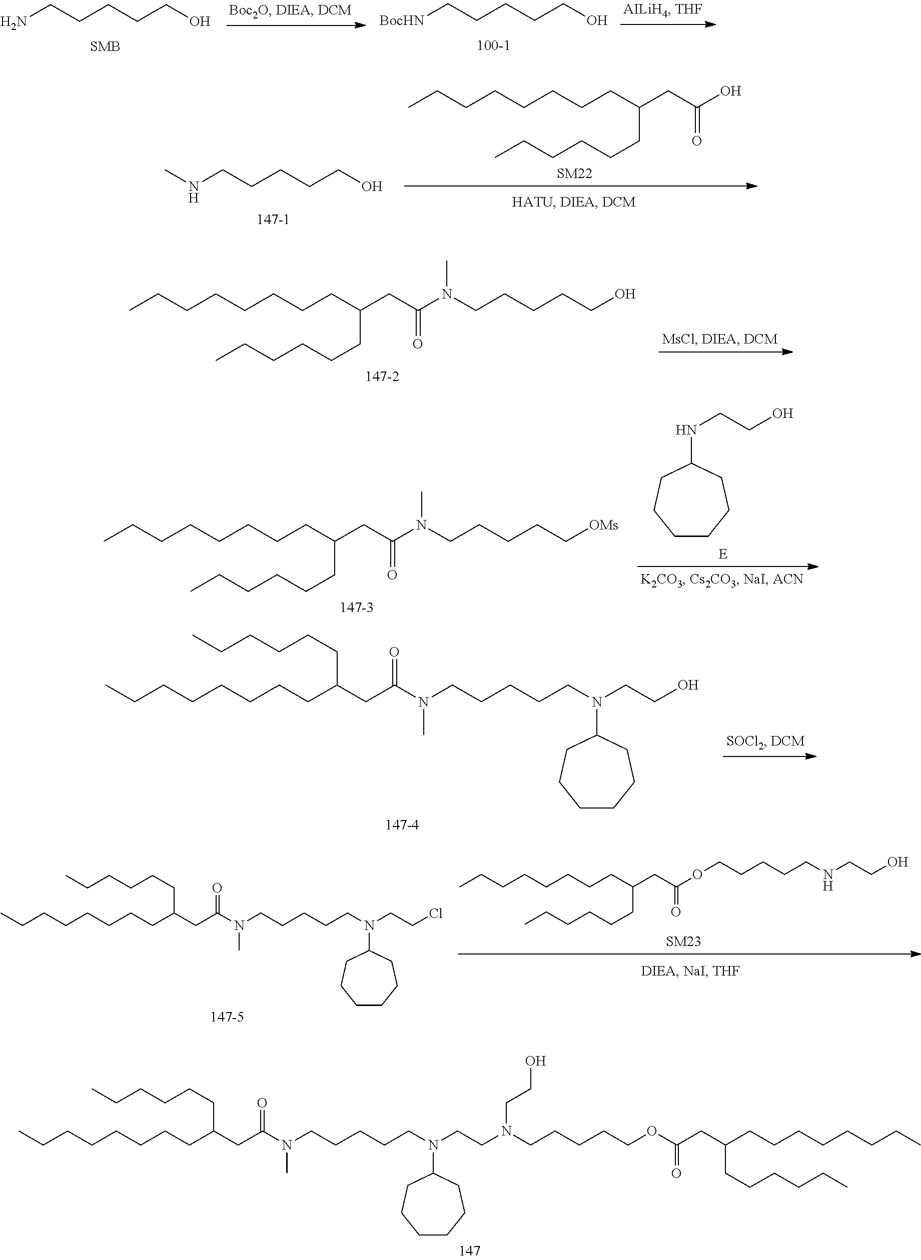

### Step 1: Preparation of Compound 100-1

To a mixture of compound SM8 (1 g, 9.690 mmol, 1.0 eq.), DIEA (1.9 g, 14.54 mmol, 1.5 eq.) in DCM (15 mL) was added Boc₂O (2.5 g, 11.63 mmol, 1.2 eq.). The reaction mixture was stirred at RT for 16 hours. TLC showed the reaction was complete. The mixture power in water and washed with DCM. The organic was separated and dried over Na₂SO₄. Removal of solvent, FCC to get the compound 100-1 (1.7 g, 86.30%) as colorless oil.

### Step 2: Preparation of Compound 147-1

To a solution of compound 100-1 (1.7 g, 8.362 mmol, 1.0 eq.) in THF F (30 mL) was added slowly LiAlH₄ (0.64 g, 16.72 mmol, 2.0 eq.) in 0° C. The reaction mixture was stirred at reflux for 2 hours. TLC showed the reaction was complete. After being cooled to 0° C. The mixture was quenched with successive addition of water (1.3 mL), 15% aqueous NaOH (1.3 mL) and water (3.9 mL). The resulting mixture was diluted with ethyl acetate and the precipitate was removed by titration. The filtrate was evaporated under reduced pressure to get the compound 147-1 (0.8 g, 81.63%) as a yellow oil.

### Step 3: Preparation of Compound 147-2

To a mixture of compound 147-1 (300 mg, 2.559 mmol, 1.0 eq.), compound SM22 (692 mg, 2.559 mmol, 1.0 eq.), DIEA (662 mg, 5.122 mmol, 2.0 eq.) in DCM (25 mL) was added HATU (1.46 g, 3.839 mmol, 1.5 eq.). The reaction mixture was stirred at RT for 2 hours. TLC showed the reaction was complete. The mixture power in water and washed with DCM. The organic was separated and dried over Na₂SO₄. Removal of solvent, FCC to get the compound 147-2 (800 mg, crude) as colorless oil.

### Step 4: Preparation of Compound 147-3

To a mixture of compound 147-2 (800 mg, 2.165 mmol, 1.0 eq.), DIEA (560 mg, 4.329 mmol, 2.0 eq.) in DCM (15 mL) was added MsCl (248 mg, 2.165 mmol, 1.0 eq.) at 0° C. under N₂. The reaction mixture was stirred at 0° C. for 2 hours. TLC showed the reaction was complete. The mixture power in water and washed with DCM. The organic was separated and dried over Na₂SO₄. Removal of solvent, FCC to get the compound 147-3 (550 mg, 56.74%) as a yellow oil.

### Step 5: Preparation of Compound 147-4

To a solution of compound 147-3 (550 mg, 1.229 mmol, 1.0 eq.) in ACN (15 mL) was added compound E (232 mg, 1.474 mmol, 1.2 eq.), K₂CO₃ (509 mg, 3.686 mmol, 3.0 eq.), Cs₂CO₃ (120 mg, 0.3686 mmol, 0.3 eq.), NaI (55 mg, 0.3686 mmol, 0.3 eq.). The reaction mixture was stirred at 85° C. for 16 hours. LCMS showed the reaction was complete. Removal of solvent, FCC to get the compound 147-4 (230 mg, 36.77%) as a yellow oil. LCMS: Rt: 0.850 min; MS m/z (ESI): 509.5 [M+H]⁺.

### Step 6: Preparation of Compound 147-5

To a solution of compound 147-4 (230 mg, 0.4520 mmol, 1.0 eq.) in DCM (10 mL) was added SOCl₂ (161 mg, 1.356 mmol, 3.0 eq.). The reaction mixture was stirred at 35° C. for 16 hours. LCMS showed the reaction was complete. Removal of solvent to get the compound 147-5 (270 mg, crude) as a yellow oil. LCMS: Rt: 0.890 min; MS m/z (ESI): 527.5[M+H]⁺.

### Step 7: Preparation of Compound 147

To a mixture of compound 147-5 (270 mg, 0.4520 mmol, 1.0 eq.), DIEA (292 mg, 2.260 mmol, 5.0 eq.) in THF (15 mL) was added compound SM23 (217 mg, 0.5424 mmol, 1.2 eq.), NaI (15 mg). The reaction mixture was stirred at 70° C. for 16 hours. LCMS showed the reaction was complete. After removal of solvent, the residue was purified by pre-HPLC to give the title compound (50 mg, 12.42% yield) as a yellow oil. LCMS: Rt: 1.420 min; MS m/z (ESI): 890.8[M+H]⁺.

¹H NMR (400 MHz, CDCl₃): 0.86-0.89 (m, 12H), 1.26 (s, 48H), 1.34-1.41 (m, 6H), 1.45-1.52 (m, 10H), 1.62-1.91 (m, 11H), 2.19-2.23 (m, 4H), 2.38-2.67 (m, 11H), 2.94 (d, J=25.2 Hz, 3H), 3.24-3.37 (m, 2H), 3.52-3.54 (m, 2H), 4.04-4.07 (m, 2H).

### 6.51 Example 51: Preparation of Compound 148

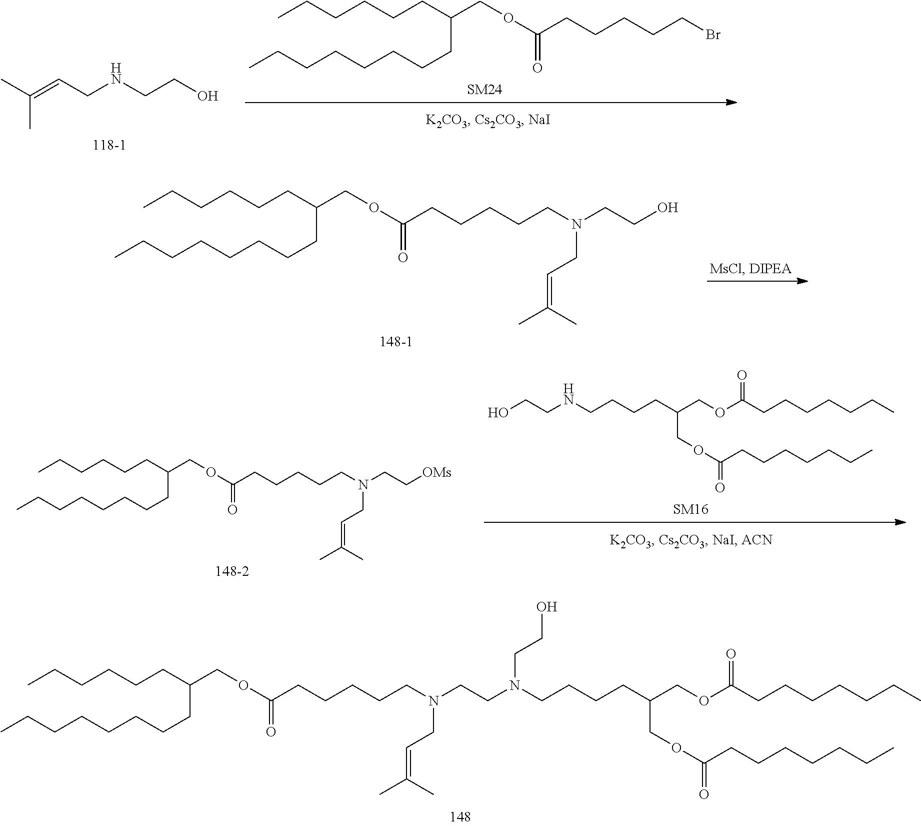

### Step 1: Preparation of Compound 148-1

To a solution of compound 118-1 (600 mg, 4.64 mmol, 2.0 eq.) and compound SM24 (973 mg, 2.32 mmol, 1.0 eq.) in ACN (40 mL) were added K₂CO₃ (962 mg, 6.96 mmol, 3.0 eq.), Cs₂CO₃ (228 mg, 0.70 mmol, 0.3 eq.) and NaI (105 mg, 0.70 mmol, 0.3 eq.). The mixture was stirred at 80° C. for 16 hours. LCMS showed the reaction was complete. The reaction mixture was concentrated and purified by column chromatography on silica gel (DCM/MeOH=25/1) to give the title compound (525 mg, 49% yield) as a yellow oil. LCMS: Rt: 0.850 min; MS m/z (ESI): 468.4 [M+H]⁺.

### Step 2: Preparation of Compound 148-2

To a solution of compound 148-1 (220 mg, 0.47 mmol, 1.0 eq.) and DIPEA (121 mg, 0.94 mmol, 2.0 eq.) in DCM (5 mL) at 0° C. was added MsCl (65 mg, 0.56 mmol, 1.2 eq.). The mixture was stirred at room temperature for 2 hours. The mixture was poured into water and extracted with DCM. The combined organic layers were washed with brine, dried over Na₂SO₄ and concentrated to give the title compound (238 mg, 93% yield) as a yellow oil. It was used in the next step without further purification. LCMS: Rt: 0.940 min; MS m/z (ESI): 486.4 [M-OMs+Cl]⁺.

### Step 3: Preparation of Compound 148

To a solution of compound 148-2 (200 mg, 0.37 mmol, 1.0 eq.) and compound SM16 (163 mg, 0.37 mmol, 1.0 eq.) in ACN (10 mL) were added K₂CO₃ (153 mg, 1.11 mmol, 3.0 eq.), Cs₂CO₃ (36 mg, 0.11 mmol, 0.3 eq.) and NaI (16 mg, 0.11 mmol, 0.3 eq.). The mixture was stirred at 80° C. for 16 hours LCMS show the reaction was completed. The mixture was concentrated and purified by prep HPLC to give the title compound (50 mg, 15% yield) as a yellow oil.

¹H NMR (400 MHz, CDCl₃) δ: 0.86-0.90 (m, 12H), 1.26-1.35 (m, 48H), 1.41-1.52 (m, 4H), 1.59-1.64 (m, 10H), 1.73-1.76 (m, 3H), 1.95-2.01 (m, 1H), 2.28-2.32 (m, 6H), 2.37-2.62 (m, 9H), 3.03-3.11 (m, 2H), 3.50-3.56 (m, 2H), 3.95-3.97 (m, 2H), 4.00-4.10 (m, 4H), 5.23-5.28 (m, 1H). LCMS: Rt: 1.470 min; MS m/z (ESI): 893.7 [M+H]⁺.

The following compounds were prepared in analogous fashion as Compound 148, using corresponding starting material.

| Compound | Characterization |
|---|---|
| 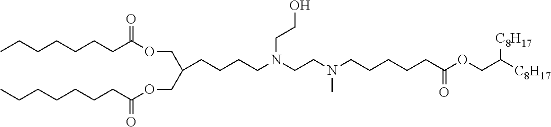 | ¹H NMR (400 MHz, CDCl₃) δ: 0.86-0.90 (m, 12H), 1.26-1.30 (m, 48 H), 1.36- 1.42(m, 4H), 1.54-1.57 (m, 6H), 1.94-2.03 (m, 1H), 2.22(s, 3H), 2.30-2.35 (m, 8H), 2.39-2.42 (m, 2H), 2.49-2.51 (m, 2H), 2.53-2.60 (m, 4H). 3.50-3.53 (m, 2H), 3.95-3.97 (m, 2H,) 4.00-4.10 (m, 4H). LCMS: Rt: 1.620 min; MS m/z (ESI): 839.6 [M + H]⁺. |
| Compound 156 | |
| 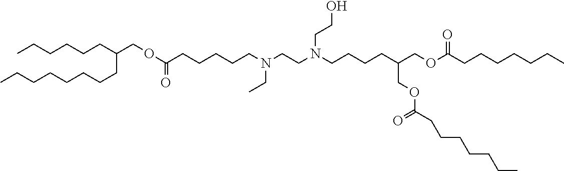 [SMILES: CCCCCCCCC(CCCCCC)COC(=O)CCCCCN(CC)CCN(CCO)CCCCC(COC(=O)CCCCCCC)COC(=O)CCCCCCC] | ¹H NMR (400 MHz, CDCl₃) δ: 0.75-0.97 (m, 12H), 1.17-1.40 (m, 47H), 1.41-1.54 (m, 3H), 1.55-1.73 (m, 8H), 1.74-2.22 (m, 7H), 2.23-2.40 (m, 7H), 2.41-2.79 (m, 8H), 3.45-3.76 (m, 2H), 3.91-4.00 (m, 2H), 4.01-4.17 (m, 4H). LCMS: Rt: 1.030 min; MS m/z (ESI): 853.8 [M + H]⁺. |
| Compound 157 | |

### 6.52 Example 52: Preparation of Compound 149

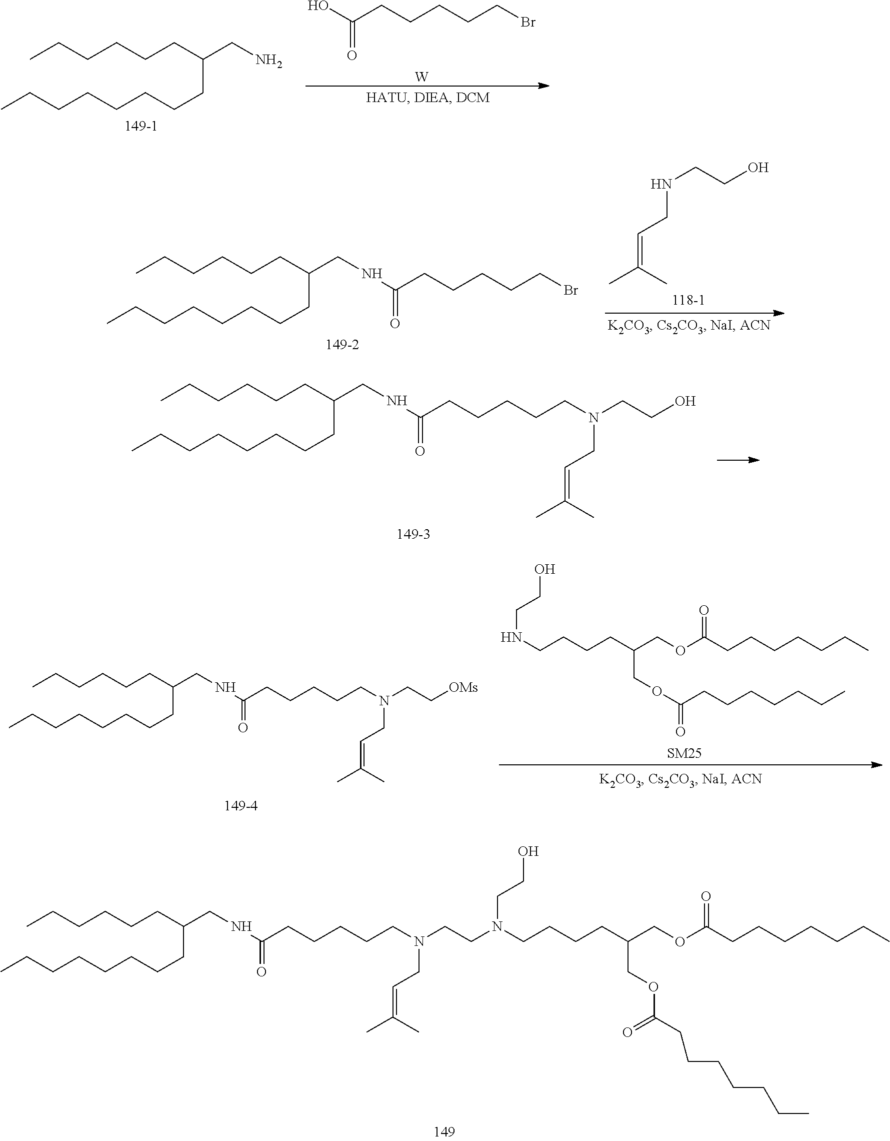

### Step 1: Preparation of Compound 149-2

A mixture of compound 149-1 (885 mg, 4.56 mmol, 1.1 eq.), compound W (1.0 g, 4.15 mmol, 1.0 eq.), HATU (1.9 g, 4.98 mmol, 1.2 eq.) and DIEA (1.6 g, 4.98 mmol, 1.2 eq.) in DCM (20 mL) was stirred for 16 hours at RT. TLC showed the reaction was completed. The mixture was concentrated and purified by column chromatography silica gel (EA:PE=0% to 5%) to give the compound 149-2 (1.2 g, 63% yield) as colorless oil.

### Step 2: Preparation of Compound 149-3

A mixture of compound 149-2 (500 mg, 1.20 mmol, 1.0 eq.), compound 118-1 (170 mg, 1.31 mmol, 1.1 eq.), K₂CO₃ (497 mg, 3.60 mmol, 3.0 eq.), Cs₂CO₃ (117 mg, 0.36 mmol, 0.3 eq.) and NaI (17 mg, 0.12 mmol, 0.1 eq.) in ACN (10 mL) was stirred overnight at 85° C. The mixture was concentrated and purified by column chromatography silica gel (MeOH:DCM=0% to 10%) to give the desired product 149-3 (300 mg, 54% yield) as a yellow oil. LCMS: Rt: 0.820 min; MS m/z (ESI): 467.4 [M+H]⁺.

### Step 3: Preparation of Compound 149-4

To a solution of compound 149-3 (280 mg, 0.60 mmol, 1.0 eq.) was dissolved in DCM (10 mL) was added MsCl (82 mg, 0.72 mmol, 1.2 eq.) at RT. The mixture was stirred for 1 hours at RT. LCMS show the reaction was completed, the mixture was evaporated under reduced pressure to provide compound 149-4 (200 mg, crude) as a yellow oil. LCMS: Rt: 0.830 min; MS m/z (ESI): 449.4 [M-OMs]⁺.

### Step 4: Preparation of Compound 149

A mixture of compound SM25 (200 mg, 0.41 mmol, 1.2 eq.), compound 149-4 (182 mg, 0.41 mmol, 1.0 eq.), K₂CO₃ (170 mg, 1.23 mmol, 3.0 eq.), Cs₂CO₃ (40 mg, 0.12 mmol, 0.3 eq.) and NaI (5.6 mg, 0.04 mmol, 0.1 eq.) in ACN (10 mL) was stirred overnight at 85° C. LCMS show the reaction was completed, The residual was purified by prep-HPLC to give the desired product 149 (62 mg, 17% yield) as a yellow oil. LCMS: Rt: 0.980 min; MS m/z (ESI): 892.8 [M+H]⁺.

¹H NMR (400 MHz, CDCl₃) δ: 0.80-0.94 (m, 12H), 1.19-1.42 (m, 42H), 1.43-1.55 (m, 4H), 1.56-1.71 (m, 12H), 1.72-1.83 (m, 4H), 1.83-2.03 (m, 2H), 2.04-2.24 (m, 4H), 2.25-2.40 (m, 6H), 2.41-2.76 (m, 9H), 3.06-3.24 (m, 3H), 3.47-3.65 (m, 2H), 4.00-4.12 (m, 4H), 5.16-5.31 (m, 1H).

The following compounds were prepared in analogous fashion as Compound 149, using corresponding starting material.

| Compound | Characterization |
|---|---|
| 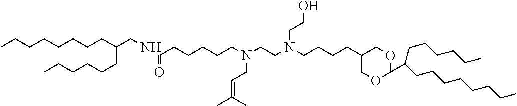 Compound 150 | ¹H NMR (400 MHz, CDCl₃) δ: 0.77-0.94 (m, 12H), 0.95-1.10 (m, 2H), 1.10-1.37 (m, 46H), 1.38-1.56 (m, 8H), 1.57-1.71 (m, 6H), 1.72-1.99 (m, 10H), 2.00-2.10 (m, 3H), 2.38-2.74 (m, 9H), 3.10-3.21 (m, 3H), 3.22-3.33 (m, 2H), 3.44-3.66 (m, 2H), 3.79-3.94 (m, 1H), 4.15-4.28 (m, 1H), 4.29-4.44 (m, 1H), 5.18-5.30 (m, 1H). LCMS: Rt: 1.740 min; MS m/z |
| | (ESI): 862.8 [M + H]⁺. |
| 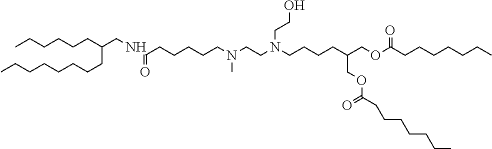 [SMILES: CCCCCCCCC(CCCCCC)CNC(=O)CCCCCN(C)CCN(CCO)CCCCC(COC(=O)CCCCCCC)COC(=O)CCCCCCC] | ¹H NMR (400 MHz, CDCl₃) δ: 0.80-0.94 (m, 12H), 1.17-1.35 (m, 47H), 1.36-1.57 (m, 6H), 1.58-1.75 (m, 6H), 1.91-2.05 (m, 1H), 2.24-2.35 (m, 6H), 2.36-2.46 (m, 6H), 2.47-2.58 (m, 2H), 2.59-2.64 (m, 4H), 3.13-3.25 (m, 2H), 3.40-3.57 (m, 2H), 4.00-4.15 (m, 4H), 5.54-5.68 (m, 1H). LCMS: Rt: 0.990 min; MS m/z (ESI): 838.6 [M + H]⁺. |
| Compound 158 | |
| 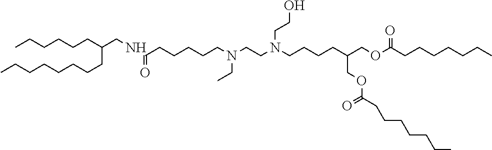 [SMILES: CCCCCCCCC(CCCCCC)CNC(=O)CCCCCN(CC)CCN(CCO)CCCCC(COC(=O)CCCCCCC)COC(=O)CCCCCCC] | ¹H NMR (400 MHz, CDCl₃) δ: 0.78-0.96 (m, 12H), 0.97-1.11 (m, 4H), 1.12-1.35 (m, 33H), 1.36-1.53 (m, 6H), 1.54-1.79 (m, 14H), 1.88-2.07 (m, 2H), 2.08-2. 23 (m, 2H), 2.24-2.38 (m, 6H), 2.39-2.61 (m, 12H), 3.10-3.23 (m, 2H), 3.46-3.60 (m, 2H), 3.97-4.13 (m, 5H), 5.53-5.71 (m, 1H). LCMS: Rt: 0.960 min; MS m/z (ESI): 852.7 [M + H]⁺. |
| Compound 159 | |

### 6.53 Example 53: Preparation of Compound 151

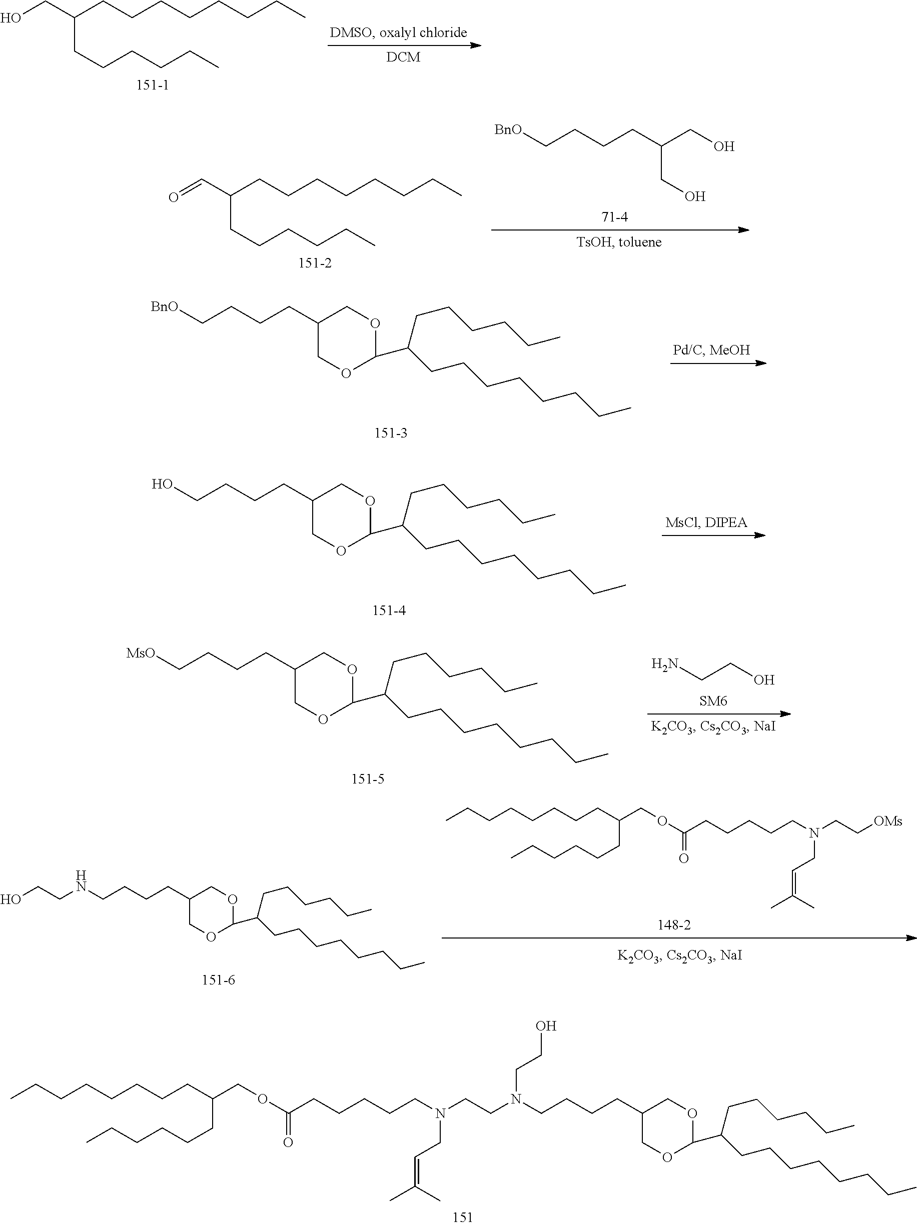

### Step 1: Preparation of Compound 151-2

To a solution of DMSO (3.2 g, 41.2 mmol, 2.0 eq.) in DCM (120 mL) at −78° C. under N₂ was added a solution of oxalyl chloride (2.9 g, 22.7 mmol, 1.1 eq.) in DCM (20 mL) dropwise. The mixture was stirred for 30 min and then compound 151-1 (5.0 g, 20.6 mmol, 1.0 eq.) was added dropwise at −78° C. The mixture was stirred for 60 min at −78° C. TEA (6.3 g, 61.8 mmol, 3.0 eq.) was added and the mixture was allowed to warm to RT. The mixture was poured into water and extracted with DCM. The combined organic layers were washed with brine, dried over Na₂SO₄ and concentrated. The residue was purified by column chromatography on silica gel (PE/EA=10/1) to give the title compound (3.2 g, 65% yield) as colorless oil.

### Step 2: Preparation of Compound 151-3

To a solution of compound 151-2 (878 mg, 3.65 mmol, 1.0 eq.) and compound 71-4 (870 mg, 3.65 mmol, 1.0 eq.) in toluene (30 mL) was added p-TsOH (70 mg, 0.37 mmol, 0.1 eq.). The mixture was stirred at 40° C. for 16 hours. The mixture was diluted with ethyl acetate and washed with Saturated NaHCO₃ aqueous solution. The organic layer was washed with brine, dried over Na₂SO₄ and concentrated. The residue was purified by column chromatography on silica gel (PE/EA=100/1) to give the title compound (1.2 g, 71% yield) as colorless oil.

### Step 3: Preparation of Compound 151-4

To a solution of compound 151-3 (1.2 g, 2.60 mmol, 1.0 eq.) in ethyl acetate (25 mL) was added Pd/C (120 mg). The mixture was stirred at 35° C. under H₂ for 16 hours. The mixture was filtered through a pad of Celite and washed with EA. The filtration was concentrated and purified by column chromatography on silica gel (PE/EA=5/1) to give the title compound (610 mg, 63% yield) as colorless oil.

### Step 4: Preparation of Compound 151-5

To a solution of compound 151-4 (610 mg, 1.65 mmol, 1.0 eq.) and DIPEA (426 mg, 3.30 mmol, 2.0 eq.) in DCM (20 mL) at 0° C. was added MsCl (227 mg, 1.98 mmol, 1.2 eq.). The mixture was stirred at room temperature for 2 hours. The mixture was poured into water and extracted with DCM. The combined organic layers were washed with brine, dried over Na₂SO₄ and concentrated to give the title compound (640 mg, 87% yield) as a yellow oil. It was used in the next step without further purification.

### Step 5: Preparation of Compound 151-6

To a solution of compound 151-5 (640 mg, 1.43 mmol, 1.0 eq.) and compound SM6 (178 mg, 2.86 mmol, 2.0 eq.) in ACN (28 mL) were added K₂CO₃ (593 mg, 4.29 mmol, 3.0 eq.), Cs₂CO₃ (140 mg, 0.43 mmol, 0.3 eq.) and NaI (64 mg, 0.43 mmol, 0.3 eq.). The mixture was stirred at 80° C. for 16 hours. LCMS showed the reaction was complete. The mixture was poured into water and extracted with EA. The combined organic layers were washed with brine, dried over Na₂SO₄ and concentrated. The residue was purified by column chromatography on silica gel (DCM/MeOH=10/1) to give the title compound (362 mg, 61% yield) as a yellow oil. LCMS: Rt: 0.870 min; MS m/z (ESI): 414.4[M+H]⁺.

### Step 6: Preparation of Compound 151

To a solution of compound 151-6 (180 mg, 0.44 mmol, 1.0 eq.) and compound 148-2 (240 mg, 0.44 mmol, 1.0 eq.) in ACN (15 mL) were added K₂CO₃ (182 mg, 1.32 mmol, 3.0 eq.), Cs₂CO₃ (42 mg, 0.13 mmol, 0.3 eq.) and NaI (19 mg, 0.13 mmol, 0.3 eq.). The mixture was stirred at 80° C. for 16 hours LCMS show the reaction was completed. The mixture was concentrated and purified by prep HPLC to give the title compound (36 mg, 10% yield) as a yellow oil. LCMS: Rt: 1.710 min; MS m/z (ESI): 863.8 [M+H]⁺.

¹H NMR (400 MHz, CDCl₃) δ: 0.86-0.89 (m, 12H), 0.96-0.99 (m, 2H), 1.26-1.38 (m, 51H), 1.46-1.59 (m, 6H), 1.62-1.86 (m, 10H), 1.95-1.99 (m, 1H), 2.28-2.30 (m, 2H), 2.41-2.71 (m, 9H), 2.96-3.15 (m, 2H), 3.24-3.30 (m, 2H), 3.48-3.59 (m, 2H), 3.80-3.85 (m, 1H), 3.91-3.97 (m, 2H), 4.05-4.09 (m, 2H), 4.30-4.45 (m, 1H), 5.22-5.28 (m, 1H).

### 6.54 Example 54: Preparation of Compound 152

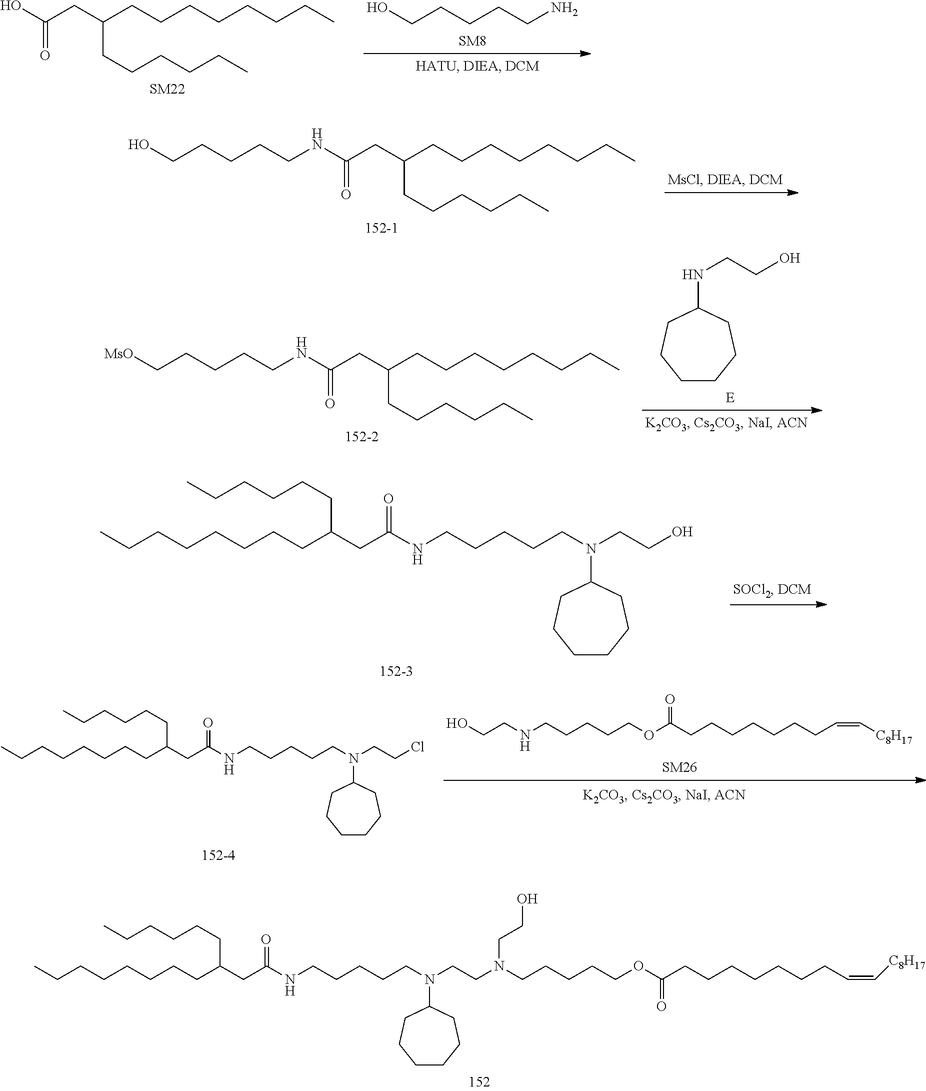

### Step 1: Preparation of Compound 152-1

To a mixture of compound SM22 (1 g, 3.697 mmol, 1.0 eq.), compound SM8 (1.2 g, 4.436 mmol, 1.2 eq.), DIEA (0.96 g, 7.394 mmol, 2.0 eq.) in DCM (15 mL) was added HATU (2.1 g, 5.546 mmol, 1.5 eq.). The reaction mixture was stirred at RT for 2 hours. TLC showed the reaction was complete. The mixture power in water and washed with DCM. The organic was separated and dried over Na₂SO₄. Removal of solvent, FCC to get the compound 152-1 (1.2 g, 91.28%) as a yellow oil.

### Step 2: Preparation of Compound 152-2

To a mixture of compound 152-1 (1.2 g, 3.375 mmol, 1.0 eq.), DIEA (0.87 g, 6.750 mmol, 2.0 eq.) in DCM (20 mL) was added MsCl (0.46 g, 4.049 mmol, 1.2 eq.) at 0° C. under N₂. The reaction mixture was stirred at 0° C. for 2 hours. TLC showed the reaction was complete. The mixture power in water and washed with DCM. The organic was separated and dried over Na₂SO₄. Removal of solvent, FCC to get the compound 152-2 (1 g, 64.18%) as a yellow oil.

### Step 3: Preparation of Compound 152-3

To a solution of compound 152-2 (1 g, 2.166 mmol, 1.0 eq.) in ACN (20 mL) was added compound E (0.4 g, 2.599 mmol, 1.2 eq.), K₂CO₃ (0.9 g, 6.498 mmol, 3.0 eq.), Cs₂CO₃ (0.21 g, 0.6498 mmol, 0.3 eq.), NaI (0.1 g, 0.6498 mmol, 0.3 eq.). The reaction mixture was stirred at 80° C. for 16 hours. LCMS showed the reaction was complete. Removal of solvent, FCC to get the compound 152-3 (600 mg, 55.98%) as a yellow oil. LCMS: Rt: 0.870 min; MS m/z (ESI): 495.4 [M+H]⁺.

### Step 4: Preparation of Compound 152-4

To a solution of compound 152-3 (600 mg, 1.213 mmol, 1.0 eq.) in DCM (15 mL) was added SOCl₂ (433 mg, 3.638 mmol, 3.0 eq.). The reaction mixture was stirred at 35° C. for 16 hours. LCMS showed the reaction was complete. Removal of solvent to get the compound 152-4 (650 mg, crude) as a yellow oil. LCMS: Rt: 0.910 min; MS m/z (ESI): 513.4[M+H]⁺.

### Step 5: Preparation of Compound 152

To a mixture of compound 152-4 (200 mg, 0.3896 mmol, 1.0 eq.) in ACN (10 mL) was added compound SM26 (192 mg, 0.4676 mmol, 1.2 eq.), K₂CO₃ (162 mg, 1.169 mmol, 3.0 eq.), Cs₂CO₃ (38 mg, 0.1169 mmol, 0.3 eq.), NaI (18 mg, 0.1169 mmol, 0.3 eq.). The reaction mixture was stirred at 80° C. for 16 hours. LCMS showed the reaction was complete. After removal of solvent, the residue was purified by pre-HPLC to give the title compound (52 mg, 15.02% yield) as a yellow oil. LCMS: Rt: 1.440 min; MS m/z (ESI): 888.8 [M+H].

¹H NMR (400 MHz, CDCl₃): 0.86-0.89 (m, 9H), 1.28 (d, J=19.6 Hz, 49H), 1.45-1.52 (m, 9H), 1.62-1.68 (m, 10H), 1.78-1.86 (m, 4H), 1.99-2.07 (m, 6H), 2.27-2.31 (m, 2H), 2.50-2.68 (m, 11H), 3.22-3.26 (m, 2H), 3.52-3.55 (m, 2H), 4.04-4.07 (m, 2H), 5.33-5.36 (m, 2H), 5.85 (s, 1H).

The following compounds were prepared in analogous fashion as Compound 152, using corresponding starting material.

| Compound | Characterization |
|---|---|
| 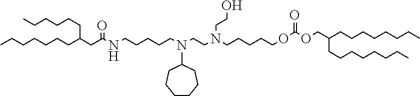 Compound 153 | 1H NMR (400 MHz, CDCl₃): 0.86-0.90 (m, 12H), 1.26 (s, 53H), 1.43-1.52 (m, 10H), 1.60-1.71 (m, 12H), 1.80-1.86 (m, 4H), 2.05-2.07 (m, 2H), 2.37-2.70 (m, 11H), 3.22-3.26 (m, 2H), 3.52 (s, 2H), 4.03 (d, J = 5.6 Hz, 2H), 4.10-4.14 (m, 2H), 5.84 (s, 1H). LCMS: Rt: 1.390 min; MS m/z (ESI): 920.9 [M + H]⁺. |

### 6.55 Example 55: Preparation of Compound 161

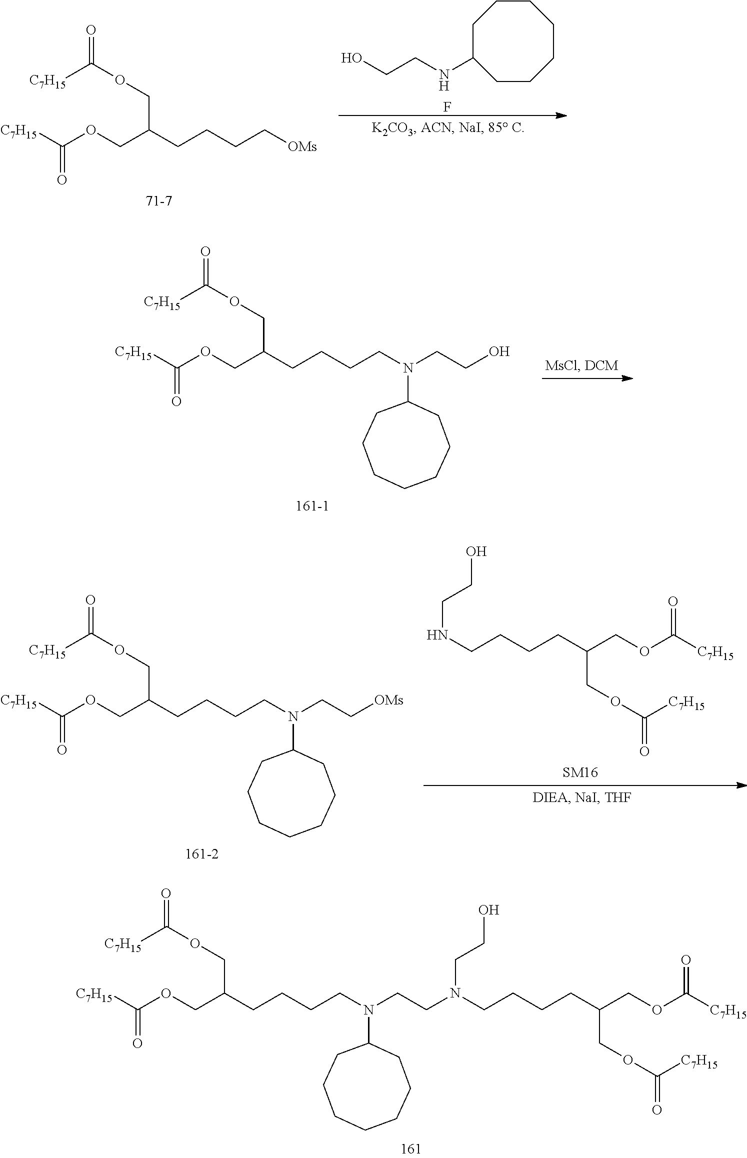

### Step 1: Preparation of Compound 161-1

To a solution of compound 71-7 (500 mg, 1.044 mmol, 1.0 eq.) and compound F (215 mg, 1.253 mmol, 1.2 eq.) in ACN (10 mL) were added K₂CO₃ (433 mg, 3.132 mmol, 3.0 eq.), Cs₂CO₃ (102 mg, 0.3132 mmol, 0.3 eq.) and NaI (51 mg, 0.3132 mmol, 0.3 eq.). The mixture was stirred at 85° C. for 16 hours. LCMS showed the reaction was complete. The reaction mixture was concentrated and purified by column chromatography on silica gel (DCM/MeOH=10/1) to give the title compound (300 mg, 51.88%) as a yellow oil. LCMS: Rt: 0.840 min; MS m/z (ESI): 554.4 [M+H]⁺.

### Step 2: Preparation of Compound 161-2

To a solution of compound 161-1 (300 mg, 0.5416 mmol, 1.0 eq.) and DIPEA (105 mg, 0.8124 mmol, 1.5 eq.) in DCM (10 mL) at 0° C. was added MsCl (74 mg, 0.6499 mmol, 1.2 eq.). The mixture was stirred still for 2 hours. The mixture was poured into water and extracted with DCM. The combined organic layers were washed with brine, dried over Na₂SO₄ and concentrated to give the title compound (340 mg, crude) as a yellow oil.

### Step 3: Preparation of Compound 161

To a solution of compound 161-2 (340 mg, 0.5380 mmol, 1.04 eq.) and compound SM16 (230 mg, 0.5184 mmol, 1.0 eq.) in THF (15 mL) were added DIEA (335 mg, 2.592 mmol, 5.0 eq.) and NaI (15 mg). The mixture was stirred at 70° C. for 16 hours. LCMS showed the reaction was completed. The mixture was concentrated and purified by prep HPLC to give the title compound (60 mg, 11.82% yield) as a yellow oil. ¹H NMR (400 MHz, CDCl₃) δ: 0.86-0.90 (m, 12H), 1.29 (s, 31H), 1.32-1.35 (m, 7H), 1.42-1.44 (m, 11H), 1.59-1.72 (m, 19H), 1.95-2.00 (m, 2H), 2.28-2.32 (m, 8H), 2.35-2.42 (m, 3H), 2.47-2.60 (m, 6H), 2.80 (s, 1H), 3.52-3.54 (m, 2H), 4.00-4.10 (m, 8H). LCMS: Rt: 1.145 min; MS m/z (ESI): 979.7 [M+H]⁺.

The following compounds were prepared in analogous fashion as Compound 161, using corresponding starting material.

| Compound | Characterization |
|---|---|
| 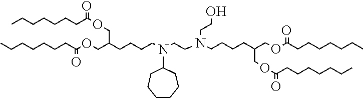 [SMILES: CCCCCCCC(=O)OCC(CCCCN(CCO)CCN(CCCCC(COC(=O)CCCCCCC)COC(=O)CCCCCCC)C1CCCCCC1)COC(=O)CCCCCCC] | ¹H NMR (400 MHz, CDCl₃) δ: 0.73-0.89 (m, 12H), 1.10-1.40 (m, 50H), 1.41-1.63 (m, 12H), 1.64-1.81 (m, 2H), 1.82-2.00 (m, 2H), 2.14-2.31 (m, 9H), 2.32-2.66 (m, 10H), 3.38-3.51 (m, 2H), 3.88-4.07 (m, 9H). LCMS: Rt: 1.010 min; MS m/z (ESI): 965.7 [M + H]⁺. |
| Compound 160 | |
| 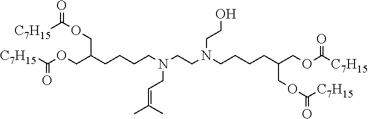 | ¹H NMR (400 MHz, CDCl₃) δ: 0.86-0.90 (m, 12H), 1.27-1.73 (m, 58H), 1.95-1.97 (m, 3H), 2.28-2.59 (m, 18H), 3.06-3.08 (m, 2H), 3.51-3.53 (m, 2H), 4.02-4.07 (m, 8H), 5.22-5.06 (m, 1H). LCMS: Rt: 0.377 min; MS m/z (ESI): 937.6 [M + H]⁺. |
| Compound 162 | |
| 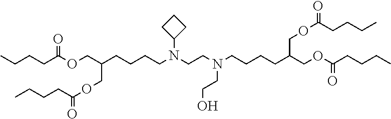 [SMILES: CCCCC(=O)OCC(CCCCN(CCO)CCN(CCCCC(COC(=O)CCCC)COC(=O)CCCC)C1CCC1)COC(=O)CCCC] | ¹H NMR (400 MHz, CDCl₃) δ: 0.86-0.96 (m, 12H), 1.23-1.39 (m, 19H), 1.40-1.49 (m, 3H), 1.53-1.68 (m, 10H), 1.84-2.04 (m, 6H), 2.20-2.34 (m, 8H), 2.36-2.64 (m, 9H), 3.01-3.12 (m, 1H), 3.49-3.58 (m, 2H), 3.96-4.13 (m, 8H). LCMS: Rt: 0.820 min; MS m/z (ESI): 755.4 [M + H]⁺. |
| Compound 163 | |
| 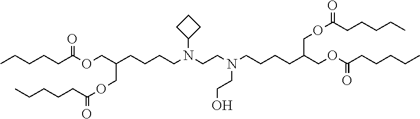 [SMILES: CCCCCC(=O)OCC(CCCCN(CCO)CCN(CCCCC(COC(=O)CCCCC)COC(=O)CCCCC)C1CCC1)COC(=O)CCCCC] | ¹H NMR (400 MHz, CDCl₃) δ: 0.84-0.94 (m, 12H), 1.21-1.49 (m, 31H), 1.55-1.69 (m, 10H), 1.84-2.06 (m, 6H), 2.20-2.34 (m, 8H), 2.35-2.65 (m, 8H), 2.99-3.13 (m, 1H), 3.55 (m, 2H), 3.94-4.14 (m, 8H). LCMS: Rt: 0.880 min; MS m/z (ESI): 811.5 [M + H]⁺. |
| Compound 164 | |
| 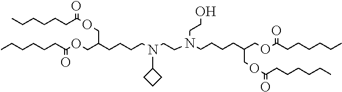 [SMILES: CCCCCCC(=O)OCC(CCCCN(CCO)CCN(CCCCC(COC(=O)CCCCCC)COC(=O)CCCCCC)C1CCC1)COC(=O)CCCCCC] Compound 165 | ¹H NMR (400 MHz, CDCl₃) δ: 0.86-0.90 (m, 12H), 1.27-1.46 (m, 37H), 1.57-1.63 (m, 10H), 1.98-2.00 (m, 6H), 2.28-2.32 (m, 8H), 2.41-2.60 (m, 10H), 3.00-3.14 (m, 1H), 3.47-3.60 (m, 2H), 4.01-4.10 (m, 8H). LCMS: Rt: 1.013 min; MS m/z (ESI): 867.6 [M + H]⁺. |
| 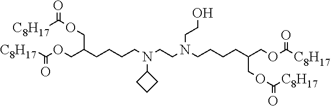 | ¹H NMR (400 MHz, CDCl₃) δ: 0.86-0.90 (m, 12H), 1.27-1.65 (m, 62H), 1.87-1.99 (m, 7H), 2.28-2.42 (m, 12H), 2.49-2.61 (m, 6H), 3.05-3.08 (m, 1H), 3.52-3.54 (m, 2H), 4.01-4.10 (m, 8H). LCMS: Rt: 0.520 min; MS m/z (ESI): 979.6 [M + H]⁺. |
| Compound 166 | |
| 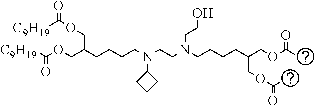 | ¹H NMR (400 MHz, CDCl₃) δ: 0.86-0.90 (m, 12H), 1.29-1.52 (m, 61H), 1.52-1.63 (m, 10H), 1.82-2.09 (m, 7H), 2.28-2.33 (m, 8H), 2.40-2.68 (m, 9H), 3.04-3.11 (m, 1H), 3.50-3.62 (m, 2H), 4.02-4.07 (m, 8H). LCMS: Rt: 1.850 min; MS m/z (ESI): 1035.7 [M + H]⁺. |
| Compound 167 | |
| 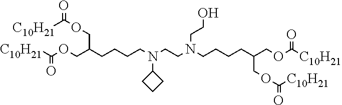 | ¹H NMR (400 MHz, CDCl₃) δ: 0.86-0.90 (m, 12H), 1.26 (s, 59H), 1.34-1.45 (m, 10H), 1.57-1.68 (m, 10H), 1.87-1.99 (m, 6H), 2.28-2.61 (m, 18H), 3.03-3.11 (m, 1H), 3.53-3.56 (m, 2H), 4.00-4.10 (m, 8H). LCMS: Rt: 0.093 min; MS m/z (ESI): 1091.7 [M + H]⁺. |
| Compound 168 | |
| 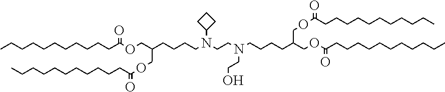 [SMILES: CCCCCCCCCCCC(=O)OCC(CCCCN(CCO)CCN(CCCCC(COC(=O)CCCCCCCCCCC)COC(=O)CCCCCCCCCCC)C1CCC1)COC(=O)CCCCCCCCCCC] Compound 169 | ¹H NMR (400 MHz, CDCl₃) δ: 0.86-0.90 (m, 12H), 1.11-1.39 (m, 71H), 1.40-1.50 (m, 4H), 1.64-1.75 (m, 14H), 1.75-2.09 (m, 6H), 2.28-2.32 (m, 8H), 2.33-2.39 (m, 2H), 2.42-2.52 (m, 4H), 2.54-2.60 (m, 2H), 3.04-3.11 (m, 1H), 3.54-3.65 (m, 2H), 4.01-4.11 (m, 8H). LCMS: Rt: 1.000 min; MS m/z (ESI): 1147.7 |
| | [M + H]⁺. |

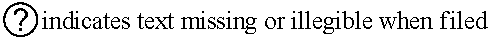

### 6.56 Example 56: Preparation of Compound 170

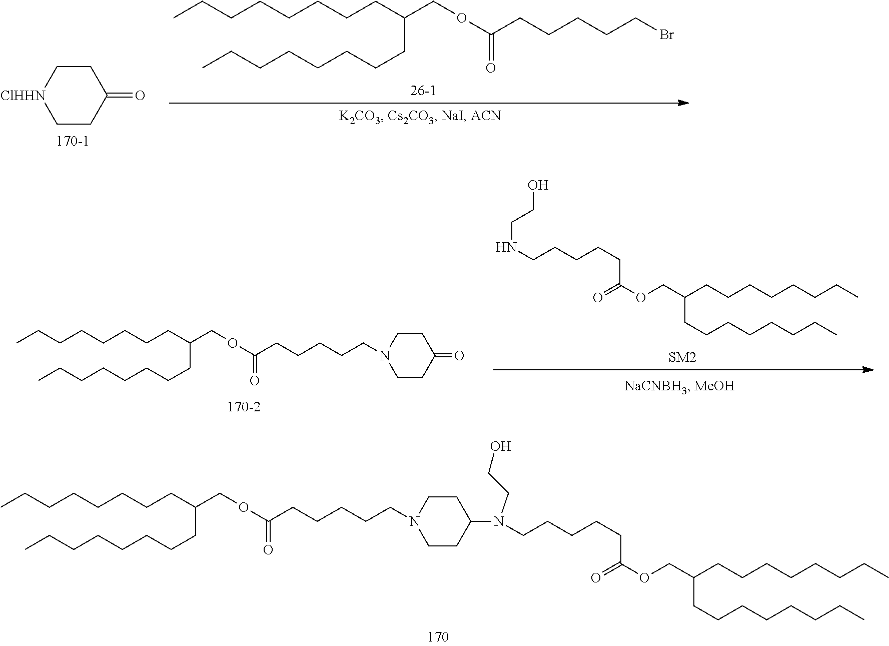

### Step 1: Preparation of Compound 170-2

To a solution of compound 170-1 (500 mg, 3.69 mmol, 1.0 eq.) and compound 26-1 (1.3 g, 2.95 mmol, 1.0 eq.) in ACN (60 mL) were added K₂CO₃ (1.5 g, 11.07 mmol, 3.0 eq.), Cs₂CO₃ (361 mg, 1.11 mmol, 0.3 eq.) and NaI (166 mg, 1.11 mmol, 0.3 eq.). The mixture was stirred at 80° C. for 16 hours. LCMS showed the reaction was complete. The reaction mixture was concentrated and purified by column chromatography on silica gel (DCM/MeOH=50/1) to give the title compound (630 mg, 37% yield) as a yellow oil. LCMS: Rt: 1.007 min; MS m/z (ESI): 466.3[M+H]⁺.

### Step 2: Preparation of Compound 170

To a solution of compound 170-2 (300 mg, 0.64 mmol, 1.0 eq.) in MeOH (10 mL) were added compound SM2 (219 mg, 0.51 mmol, 0.8 eq.) and AcOH (1 drop). The mixture was stirred at RT for 2 hours. Then NaCNBH₃ (40 mg, 0.64 mmol, 1.0 eq.) was added and the resulting mixture was stirred at RT for 16 hours. LCMS showed the reaction was complete. The mixture was concentrated and purified by prep HPLC to give the title compound (54 mg, 12% yield) as colorless oil.

¹H NMR (400 MHz, CDCl₃) δ: 0.86-0.90 (m, 12H), 1.26-1.36 (m, 60H), 1.42-1.54 (m, 5H), 1.59-1.74 (m, 10H), 1.89-1.96 (m, 2H), 2.30-2.35 (m, 6H), 2.48-2.57 (m, 3H), 2.64-2.66 (m, 2H), 3.00-3.03 (m, 2H), 3.49-3.52 (m, 2H), 3.96-3.97 (m, 4H). LCMS: Rt: 2.360 min; MS m/z (ESI): 877.7 [M+H]⁺.

The following compounds were prepared in analogous fashion as Compound 170, using corresponding starting material.

| Compound | Characterization |
|---|---|
| 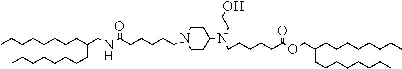 [SMILES: CCCCCCCCC(CCCCCCCC)CNC(=O)CCCCCN1CCC(N(CCO)CCCCCC(=O)OCC(CCCCCCCC)CCCCCCCC)CC1] Compound 171 | ¹H NMR (400 MHz, CDCl₃): 0.86-0.90 (m, 12H), 1.26 (s, 60H), 1.43-1.52 (m, 5H), 1.59-1.69 (m, 10H), 1.88-1.94 (m, 2H), 2.15-2.19 (m, 2H), 2.28-2.34 (m, 4H), 2.46-2.50 (m, 3H), 2.62-2.64 (m, 2H), 3.00 (d, J = 11.2 Hz, 2H), 3.16-3.19 (m, 2H), 3.47-3.50 (m, 2H), 3.96 (d, J = |
| | 5.6 Hz, 2H), 5.36 (s, 1H). LCMS: Rt: |
| | 1.300 min; MS m/z (ESI): 876.8 [M + H]⁺. |
| 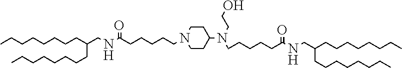 [SMILES: CCCCCCCCC(CCCCCCCC)CNC(=O)CCCCCN1CCC(N(CCO)CCCCCC(=O)NCC(CCCCCCCC)CCCCCCCC)CC1] Compound 172 | ¹H NMR (400 MHz, CDCl₃): 0.86-0.90 (m, 12H), 1.26 (s, 61H), 1.46-1.54 (m, 6H), 1.60-1.67 (m, 8H), 1.90-1.95 (m, 2H), 2.15-2.18 (m, 4H), 2.31-2.35 (m, 2H), 2.47-2.52 (m, 3H), 2.63-2.65 (m, 2H), 3.01 (d, J =11.2 Hz, 2H), 3.16-3.19 (m, 4H), 3.48-3.52 (m, 2H), 5.41-5.45 (m, |
| | 2H). LCMS: Rt: 0.960 min; MS m/z |
| | (ESI): 875.8 [M + H]⁺. |
| 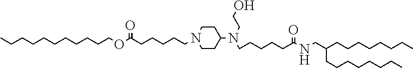 Compound 173 | ¹H NMR (400 MHz, CDCl₃): 0.86-0.90 (m, 9H), 1.26 (s, 46H), 1.43-1.66 (m, 18H), 1.85-1.90 (m, 2H), 2.28-2.32 (m, 6H), 2.46-2.52 (m, 3H), 2.62-2.64 (m, 2H), 2.98 (d, J = 10.4 Hz, 2H), 3.47-3.50 (m, 2H), 3.96 (d, J = 5.6 Hz, 2H), 4.04- 4.07 (m, 2H). LCMS: Rt: 0.970 min; MS |
| | m/z (ESI): 779.7 [M + H]⁺. |
| 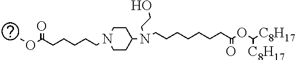 Compound 174 | ¹H NMR (400 MHz, CDCl₃): 0.87 (t, J = 8 Hz, 9H), 1.30-1.88 (m, 69H), 2.25-2.31 (m, 6H), 2.44-2.48 (m, 3H), 2.61-2.64 (m, 2H), 2.96-3.00 (m, 2H), 3.46-3.49 (m, 2H), 4.04-4.07 (m, 2H), 4.85-4.87 (m, 1H). LCMS: Rt: 0.520 min; MS m/z |
| | (ESI): 793.6 [M + H]⁺. |
| 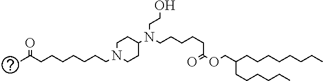 Compound 175 | ¹H NMR (400 MHz, CDCl₃): 0.87 (t, J = 8 Hz, 9H), 1.26-1.68 (m, 59H), 1.84-1.90 (m, 3H), 2.26-2.32 (m, 6H), 2.45-2.49 (m, 3H), 2.61-2.64 (m, 2H), 2.97-3.00 (m, 2H), 3.46-3.49 (m, 2H), 3.96-3.97 (m, 2H), 4.04-4.07 (m, 2H). LCMS: Rt: 0.453 min; MS m/z (ESI): 751.6 [M + H]⁺. |
| 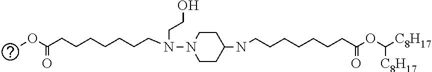 Compound 176 | ¹H NMR (400 MHz, CDCl₃): 0.87 (t, J = 8 Hz, 9H), 1.26-1.87 (m, 69H), 2.25-2.30 (m, 6H), 2.44-2.48 (m, 3H), 2.61-2.64 (m, 2H), 2.97-3.00 (m, 2H), 3.46-3.49 (m, 2H), 4.04-4.07 (m, 2H), 4.85-4.88 (m, 1H). LCMS: Rt: 0.493 min; MS m/z (ESI): 793.6 [M + H]+. |
| 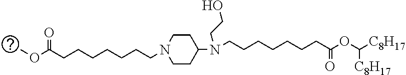 Compound 177 | ¹H NMR (400 MHz, CDCl₃): 0.87 (t, J = 8 Hz, 9H), 1.26-1.90 (m, 69H), 2.25-2.30 (m, 6H), 2.44-2.48 (m, 3H), 2.61-2.64 (m, 2H), 2.97-3.00 (m, 2H), 3.46-3.49 (m, 2H), 4.04-4.07 (m, 2H), 4.85-4.88 (m, 1H). LCMS: Rt: 0.560 min; MS m/z (ESI): 793.6 [M + H]⁺. |

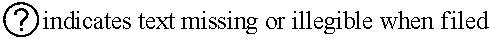

### 6.57 Example 57: Preparation of Compound 178

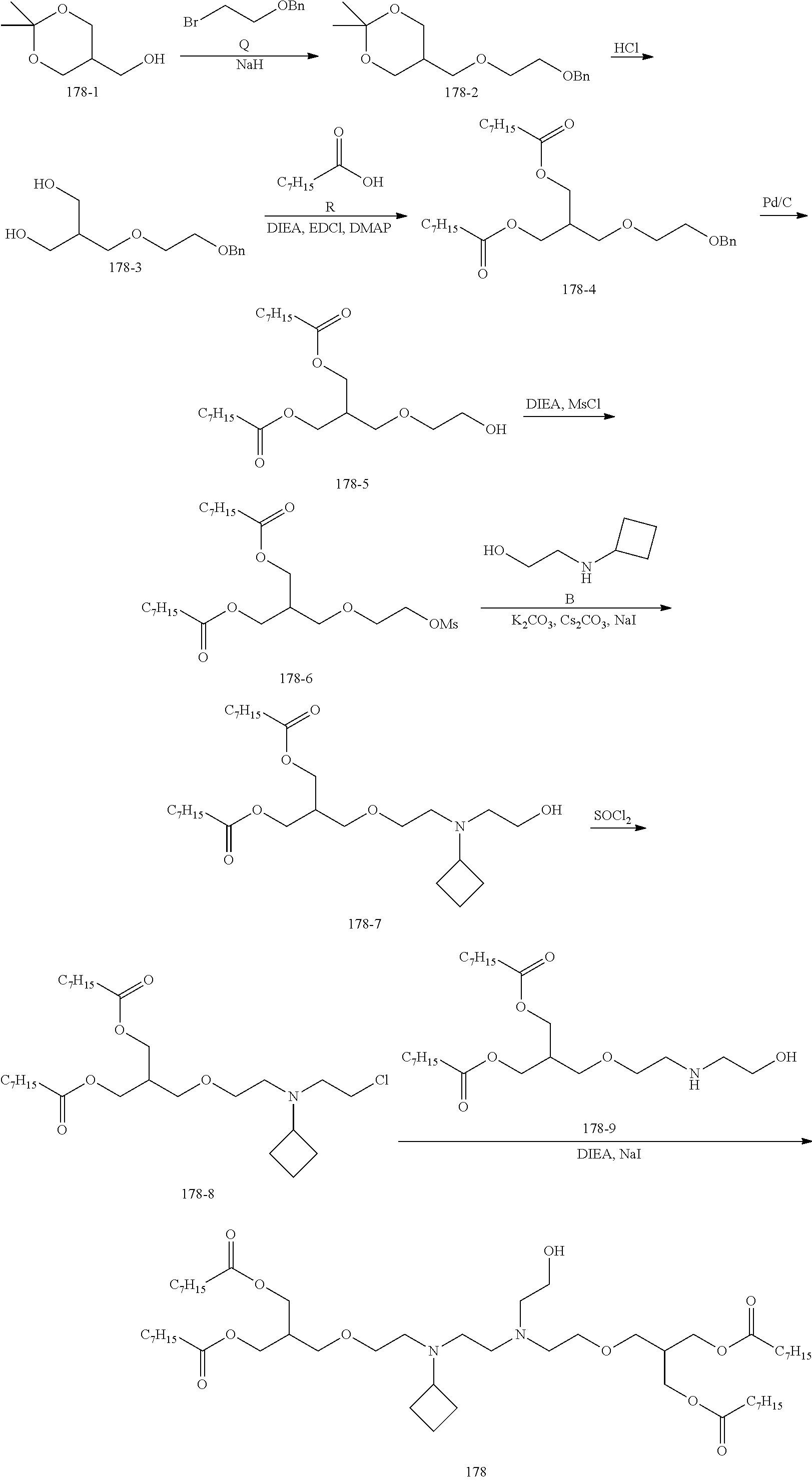

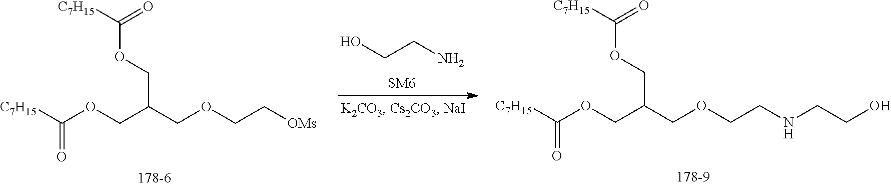

### Step 1: Preparation of Compound 178-2

To a suspension of compound 178-1 (10.0 g, 68.41 mmol, 1.0 eq.) in THF (300 mL) was added NaH (3.28 g, 82.09 mmol, 1.2 eq.) dropwise. Then the compound Q (22.07 g, 102.61 mmol, 1.5 eq.) was added dropwise and the resulting mixture was stirred at 50° C. for 10 hours. After cooling to RT, the mixture was poured into water and extracted with EA. The combined organic layers were washed with brine, dried over Na₂SO₄ and concentrated. The residue was purified by column chromatography on silica gel (PE/EA=4/1) to give the title compound (6.0 g, 31% yield) as a yellow oil.

### Step 2: Preparation of Compound 178-3

To a solution of compound 178-2 (6.0 g, 21.4 mmol, 1.0 eq.) in THF (100 mL) was added aqueous HCl (50 mL, 100 mmol, 4.7 eq.). The mixture was stirred at RT for 2 hours. The mixture was poured into water and extracted with EtOAc. The combined organic layers were washed with brine, dried over Na₂SO₄ and concentrated to give the title compound (4.5 g, 87% yield) as colorless oil.

### Step 3: Preparation of Compound 178-4

To a solution of compound 178-3 (4.5 g, 18.73 mmol, 1.0 eq.) and compound R (8.1 g, 56.18 mmol, 3.0 eq.) in DCM (200 mL) was added DIEA (12.1 g, 93.63 mmol, 5.0 eq.), EDCI (10.77 g, 56.18 mmol, 3.0 eq.) and DMAP (2.29 g, 18.73 mmol, 1.0 eq.). The mixture was stirred at 40° C. for 10 hours. The reaction mixture was concentrated and purified by column chromatography on silica gel (PE/EA=10/1) to give the title compound (7.0 g, 76% yield) as colorless oil.

### Step 4: Preparation of Compound 178-5

To a solution of compound 178-4 (7.0 g, 14.21 mmol, 1.0 eq.) in EtOAc (150 mL) was added Pd/C (1.0 g). The mixture was stirred at RT under H₂ for 10 hours. The mixture was filtered through a pad of Celite and washed with MeOH. The filtration was concentrated to give the title compound (5.1 g, 58% yield) as a yellow oil.

### Step 5: Preparation of Compound 178-6

To a solution of compound 178-5 (2.0 g, 4.97 mmol, 1.0 eq.) and DIPEA (1.93 g, 14.90 mmol, 3.0 eq.) in DCM (50 mL) was added MsCl (850 mg, 7.45 mmol, 1.5 eq.). The mixture was stirred at room temperature for 2 hours. The mixture was poured into water and extracted with DCM. The combined organic layers were washed with brine, dried over Na₂SO₄ and concentrated to give the title compound (2.0 g, 83% yield) as a yellow oil. It was used in the next step without further purification.

### Step 6: Preparation of Compound 178-7

To a solution of compound 178-6 (1.0 g, 2.08 mmol, 1.0 eq.) and compound B (480 mg, 4.16 mmol, 2.0 eq.) in ACN (30 mL) were added K₂CO₃ (860 mg, 6.24 mmol, 3.0 eq.), Cs₂CO₃ (200 mg, 0.62 mmol, 0.3 eq.) and NaI (100 mg, 0.62 mmol, 0.3 eq.). The mixture was stirred at 80° C. for 10 hours. LCMS showed the reaction was complete. The reaction mixture was concentrated and purified by column chromatography on silica gel (DCM/MeOH=10/1) to give the title compound (500 mg, 48% yield) as a yellow oil. LCMS: Rt: 0.800 min; MS m/z (ESI): 500.3[M+H]⁺.

### Step 7: Preparation of Compound 178-8

To a solution of compound 178-7 (300 mg, 0.6 mmol, 1.0 eq.) in DCM (10 mL) was added SOCl₂ (215 mg, 1.8 mmol, 3.0 eq.). The mixture was stirred at 35° C. for 10 hours. The mixture was concentrated to give the title compound (311 mg, 100% yield) as a yellow oil. LCMS: Rt: 0.467 min; MS m/z (ESI): 518.2[M+H]⁺.

### Step 8: Preparation of Compound 178-9

To a solution of compound 178-6 (1.0 g, 2.08 mmol, 1.0 eq.) and compound SM6 (250 mg, 4.16 mmol, 2.0 eq.) in ACN (30 mL) were added K₂CO₃ (860 mg, 6.24 mmol, 3.0 eq.), Cs₂CO₃ (200 mg, 0.62 mmol, 0.3 eq.) and NaI (100 mg, 0.62 mmol, 0.3 eq.). The mixture was stirred at 80° C. for 10 hours. LCMS showed the reaction was complete. The reaction mixture was concentrated and purified by column chromatography on silica gel (DCM/MeOH=10/1) to give the title compound (500 mg, 54% yield) as a yellow oil. LCMS: Rt: 0.810 min; MS m/z (ESI): 446.3[M+H]⁺.

### Step 9: Preparation of Compound 178

To a solution of compound 178-8 (200 mg, 0.38 mmol, 1.0 eq.) and compound 178-9 (180 mg, 0.38 mmol, 1.0 eq.) in THF (10 mL) were added DIPEA (150 mg, 1.16 mmol, 3.0 eq.) and NaI (60 mg, 0.38 mmol, 1.0 eq.). The mixture was stirred at 70° C. for 10 hours. LCMS show the reaction was completed. The mixture was concentrated and purified by prep HPLC to give the title compound (50 mg, 14% yield) as a yellow oil.

¹H NMR (400 MHz, CDCl₃) δ: 0.86-0.90 (m, 12H), 1.27-1.32 (m, 30H), 1.59-1.64 (m, 10H), 1.85-1.99 (m, 7H), 2.28-2.32 (m, 10H), 2.48-2.74 (m, 10H), 3.11-3.15 (m, 1H), 3.43-3.53 (m, 10H), 4.09-4.14 (m, 8H). LCMS: Rt: 1.080 min; MS m/z (ESI): 927.5[M+H]⁺.

The following compounds were prepared in analogous fashion as Compound 178, using corresponding starting material.

| Compound | Characterization |
|---|---|
| 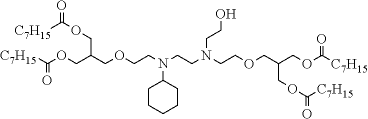 | ¹H NMR (400 MHz, CDCl₃) δ: 0.86-0.90 (m, 12H), 1.17-1.30 (m, 38H), 1.59-1.79 (m, 14H), 2.29-2.74 (m, 20H), 3.44-3.52 (m, 10H), 4.10-4.16 (m, 8H). LCMS: Rt: 1.040 min; MS m/z (ESI): 955.5 [M + H]⁺. |
| Compound 179 | |

### 6.58 Example 58: Preparation of Compound 99

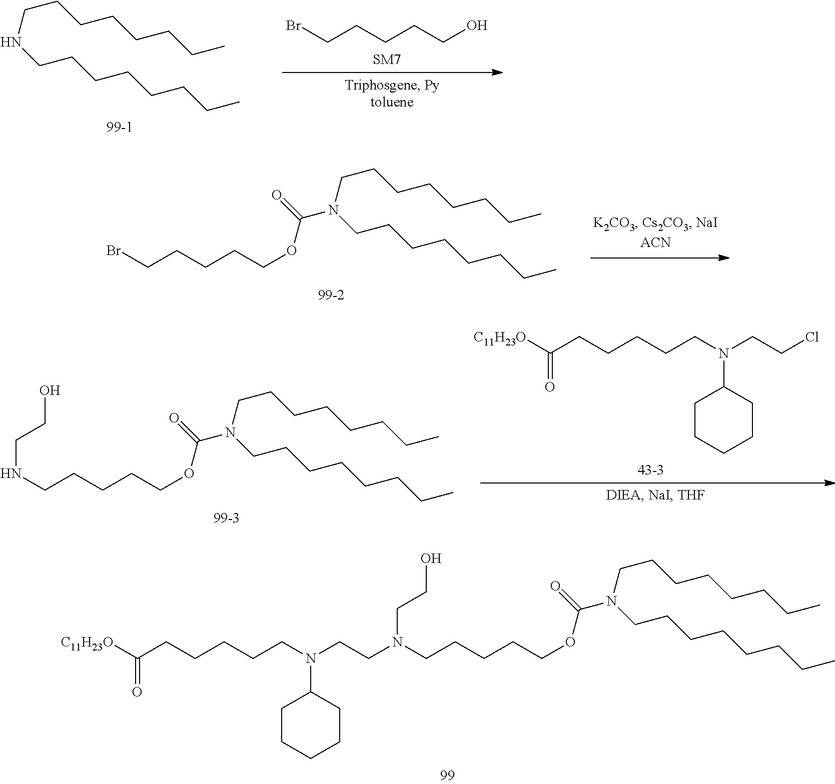

### Step 1: Preparation of Compound 99-2

To a solution of SM7 (400.0 mg, 2.0 mmol, 1.0 eq.) was dissolved in toluene (10.0 mL) at 0° C., then Py (1.1 g, 16.0 mmol, 8.0 eq.) and triphosgene (355.0 mg, 1.2 mmol, 0.6 eq.) were added. The mixture was stirred for 1 hours at RT, then compound 99-1 (578.0 mg, 2.4 mmol, 1.2 eq.) was added. The mixture was stirred for 16 hours at RT. TLC showed the reaction was completed, the mixture was poured into H₂O, exacted with EA. The mixture was evaporated under reduced pressure and purified by FCC (PE/EA=100/1-10/1) to provide compound 99-2 (0.3 g, crude) as yellow oil.

### Step 2: Preparation of Compound 99-3

To a solution of compound 99-2 (300.0 mg, 0.7 mmol, 1.0 eq.) and ethanol amine (126.0 mg, 2.01 mmol, 3.0 eq.) in ACN (15.0 mL) was added K₂CO₃ (276.0 mg, 2.01 mmol, 3.0 eq.), Cs₂CO₃ (65.0 mg, 0.2 mmol, 0.3 eq.) and NaI (10.0 mg, 0.07 mmol, 0.1 eq.) at RT. The mixture was stirred for 16 hours at 85° C. LCMS showed the reaction was completed, the mixture was evaporated under reduced pressured purified with FCC (DCM/MeOH=1/0-10/1) to provide compound 99-3 (0.16 g, crude) as colorless oil. LCMS: Rt: 0.863 min; MS m/z (ESI): 415.3 [M+H]⁺.

### Step 3: Preparation of Compound 99

To a solution of compound 99-3 (160.0 mg, 0.4 mmol, 1.0 eq.) and compound 43-3 (170.0 mg, 0.4 mmol, 3.0 eq.) in THF (5.0 mL) was added DIEA (153 mg, 1.2 mmol, 5.0 eq.) and NaI (6.0 mg, 0.04 mmol, 0.1 eq.) at 0° C. The mixture was stirred for 16 hours at 70° C. LCMS showed the reaction was completed, the mixture was evaporated under reduced pressured purified with pre-HPLC to provide compound 99 (100.0 mg, 31% yield) as yellow oil.

¹H NMR (400 MHz, CDCl₃) δ: 0.86-0.90 (m, 9H), 1.23-1.36 (m, 48H), 1.45-1.50 (m, 7H), 1.59-1.67 (m, 7H), 1.78-1.80 (m, 4H), 2.27-2.31 (m, 2H), 2.49-2.60 (m, 10H), 3.18 (s, 4H), 3.54 (s, 2H), 4.03-4.06 (m, 4H). LCMS: Rt: 1.560 min; MS m/z (ESI): 808.7 [M+H]⁺.

### 6.59 Example 59: Preparation of Compound 180

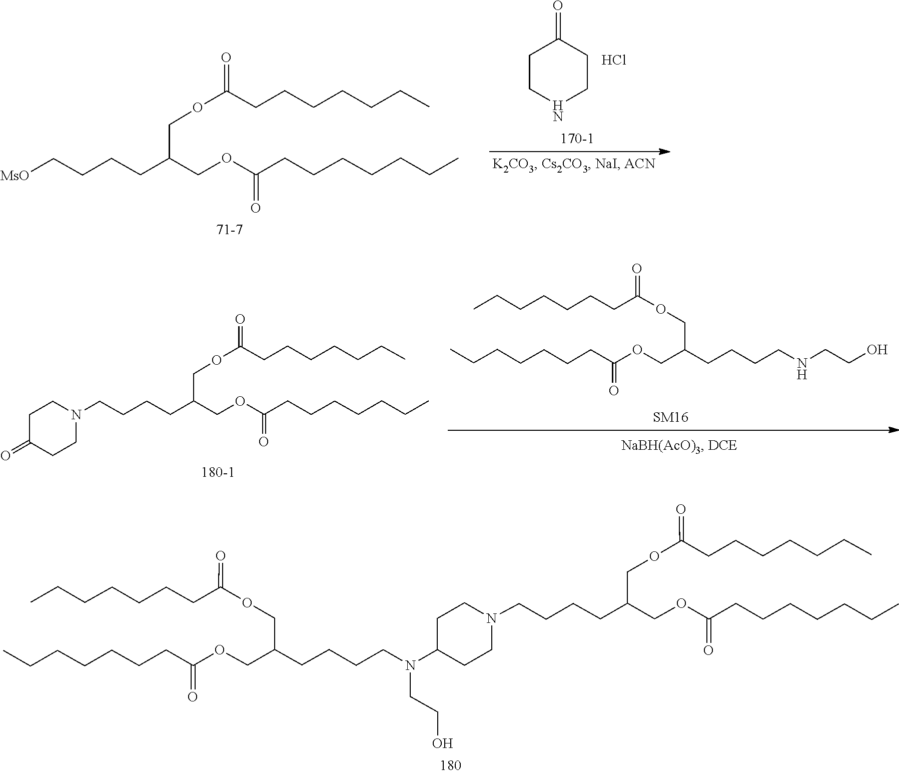 [SMILES: CCCCCCCC(=O)OCC(CCCCOS(C)(=O)=O)COC(=O)CCCCCCC ; O=C1CCNCC1 ; Cl ; CCCCCCCC(=O)OCC(CCCCN1CCC(=O)CC1)COC(=O)CCCCCCC ; CCCCCCCC(=O)OCC(CCCCNCCO)COC(=O)CCCCCCC ; CCCCCCCC(=O)OCC(CCCCN1CCC(N(CCO)CCCCC(COC(=O)CCCCCCC)COC(=O)CCCCCCC)CC1)COC(=O)CCCCCCC]

### Step 1: Preparation of Compound 180-1

A solution of compound 71-7 (1.2 g, 2.5 mmol, 1.0 eq.), compound 170-1 (500 mg, 3.7 mmol, 1.5 eq.), K₂CO₃ (1.0 g, 7.5 mmol, 3.0 eq.), Cs₂CO₃ (260 mg, 0.8 mmol, 0.3 eq.) and NaI (120 mg, 0.8 mmol, 0.3 eq.) in ACN (30 mL) was stirred at 90° C. for overnight. LCMS showed the reaction was completed. The mixture was concentrated and purified by FCC to give compound 180-1 (820 mg, 70% yield) as yellow oil.

### Step 2: Preparation of Compound 180

To a solution of compound 180-1 (200 mg, 0.42 mmol, 1.0 eq.), compound SM16 (221 mg, 0.50 mmol, 1.2 eq.) in DCE (5 mL) was stirred at rt for overnight. NaBH(AcO)₃ (176 mg, 0.83 mmol, 2.0 eq.) was added. After stirring for 4 h, LCMS showed the reaction was completed. The mixture was concentrated and purified by prep-HPLC to give compound 180 (28 mg, 7.9% yield) as yellow oil.

¹H NMR (400 MHz, CDCl₃) δ: 0.86-0.90 (m, 12H), 1.26-1.42 (m, 48H), 1.59-1.65 (m, 8H), 1.83-1.87 (m, 2H), 1.96-2.07 (m, 3H), 2.28-2.32 (m, 10H), 2.44-2.53 (m, 3H), 2.60-2.64 (m, 2H), 2.93-3.03 (m, 2H), 3.47-3.49 (m, 2H), 4.03-4.07 (m, 8H). LCMS: Rt: 0.960 min; MS m/z (ESI): 909.0 [M+H]⁺.

### 6.60 Example 60: Preparation of Compound 181

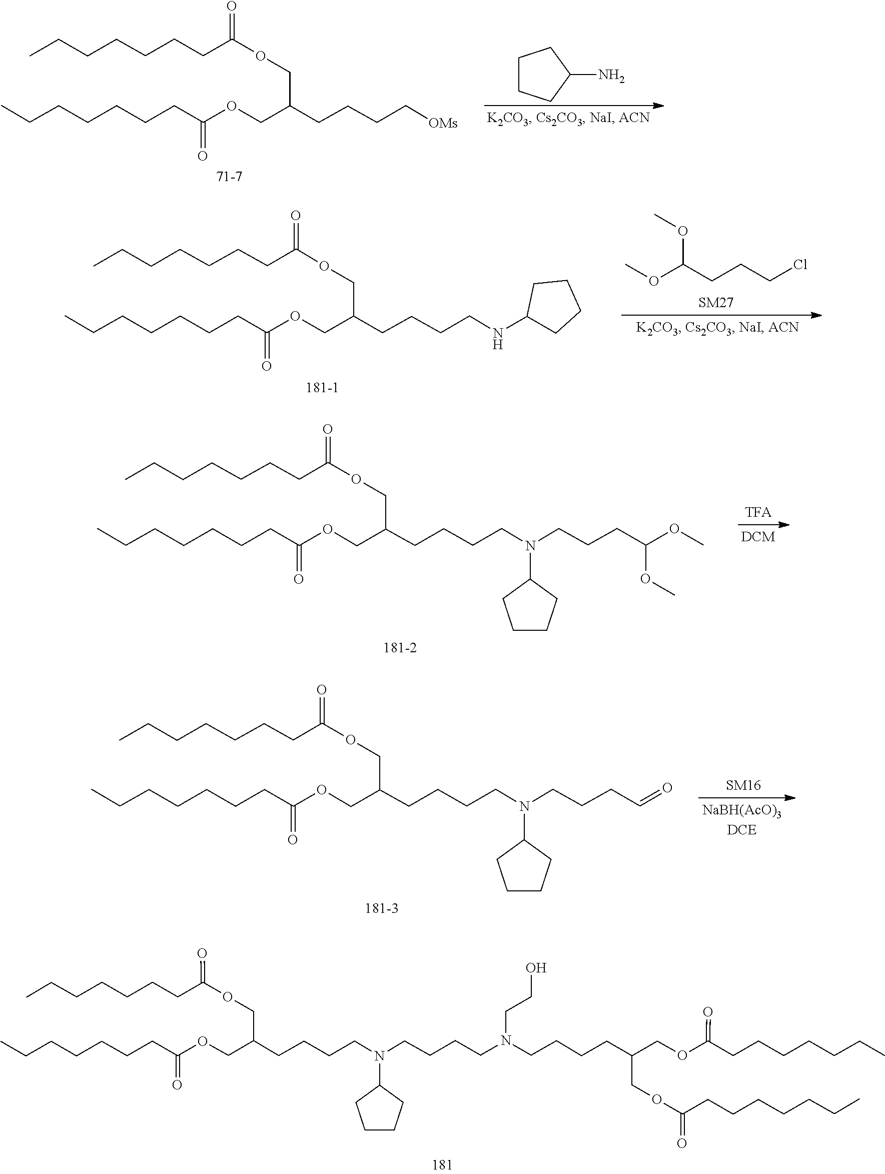

### Step 1: Preparation of Compound 181-1

To a solution of compound 71-7 (800 mg, 1.67 mmol, 1.0 eq.), cyclopentylamine (426 mg, 5.01 mmol, 3.0 eq.), DIEA (431 mg, 3.34 mmol, 2.0 eq.) in ACN (10 mL) was stirred at 70° C. for overnight. LCMS showed the reaction was completed. The mixture was concentrated and purified by FCC to give compound 181-1 (410 mg, 52.5% yield) as yellow oil.

### Step 2: Preparation of Compound 181-2

A solution of compound 181-1 (410 mg, 0.88 mmol, 1.0 eq.), compound SM27 (400 mg, 2.63 mmol, 3.0 eq.), K₂CO₃ (363 mg, 2.63 mmol, 3.0 eq.), Cs₂CO₃ (85 mg, 0.26 mmol, 0.3 eq.) and NaI (39 mg, 0.26 mmol, 0.3 eq.) in ACN (10 mL) was stirred at 90° C. for overnight. LCMS showed the reaction was completed. The mixture was concentrated and purified by FCC to give compound 181-2 (320 mg, 62.3% yield) as yellow oil.

### Step 3: Preparation of Compound 181-3

The mixture of compound 181-2 (320 mg, 0.55 mmol, 1.0 eq.), TFA (310 mg, 2.74 mmol, 5.0 eq.) in DCM (5 mL) was stirred at RT for overnight. LCMS showed the reaction was completed. The mixture was diluted with DCM and washed with water and bring, dried, concentrated, to provide crude compound 181-3 (230 mg, 77.6% yield) as yellow oil, which was used in the next step without further purification.

### Step 4: Preparation of Compound 181

A solution of compound 181-3 (230 mg, 0.43 mmol, 1.0 eq.), compound SM16 (227 mg, 0.51 mmol, 1.2 eq.) in DCE (5 mL) was stirred at rt for overnight. NaBH(AcO)₃ (186 mg, 0.86 mmol, 2.0 eq.) was added. After stirring for 4 h, LCMS showed the reaction was completed. The mixture was concentrated and purified by prep-HPLC to give compound 181 (110 mg, 26.5% yield) as yellow oil.

¹H NMR (400 MHz, CDCl₃) δ: 0.86-0.90 (m, 12H), 1.29-1.47 (m, 55H), 1.61-1.64 (m, 8H), 1.74-1.80 (m, 2H), 1.94-2.03 (m, 2H), 2.28-2.32 (m, 8H), 2.43-2.47 (m, 8H), 2.55-2.61 (m, 2H), 2.90-2.31 (m, 1H), 3.49-3.55 (m, 2H), 4.00-4.10 (m, 8H). LCMS: Rt: 1.190 min; MS m/z (ESI): 965.7 [M+H]⁺.

The following compounds were prepared in analogous fashion as Compound 181, using corresponding starting material.

| Compound | Characterization |
|---|---|
| 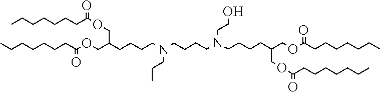 [SMILES: CCCCCCCC(=O)OCC(CCCCN(CCC)CCCCN(CCO)CCCCC(COC(=O)CCCCCCC)COC(=O)CCCCCCC)COC(=O)CCCCCCC] | 1H NMR (400 MHz, CDCl₃) δ: 0.77-0.90 (m, 15H), 1.02-1.42 (m, 42H), 1.44-1.86 (m, 18H), 1.93-2.01 (m, 2H), 2.20-2.36 (m, 9H), 2.43-2.71 (m, 10H), 3.48-3.62 (m, 2H), 3.83-4.28 (m, 8H). LCMS: Rt: 0.915 min; MS m/z (ESI): 939.7 [M + H]⁺. |
| Compound 184 | |
| 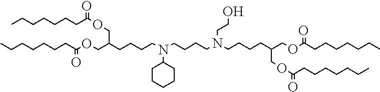 [SMILES: CCCCCCCC(=O)OCC(CCCCN(CCO)CCCCN(CCCCC(COC(=O)CCCCCCC)COC(=O)CCCCCCC)C1CCCCC1)COC(=O)CCCCCCC] Compound 185 | ¹H NMR (400 MHz, CDCl₃) δ: 0.86-0.90 (m, 12H), 1.17-1.43 (m, 47H), 1.60-1.78 (m, 20H), 1.95-2.05 (m, 3H), 2.28-2.32 (m, 8H), 2.41-2.46 (m, 8H), 2.56-2.59 (m, 2H), 3.51-3.54 (m, 2H), 4.00-4.10 (m, 8H). LCMS: Rt: 0.080 min; MS m/z (ESI): 979.7 [M + H]⁺. |
| 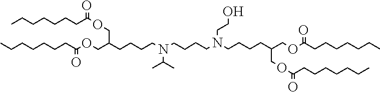 [SMILES: CCCCCCCC(=O)OCC(CCCCN(CCO)CCCCN(CCCCC(COC(=O)CCCCCCC)COC(=O)CCCCCCC)C(C)C)COC(=O)CCCCCCC] Compound 189 | ¹H NMR (400 MHz, CDCl₃) δ: 0.86-0.97 (m, 18H), 1.28-1.45 (m, 39H), 1.60-1.63 (m, 18H), 1.96-2.01 (m, 2H), 2.20-2.34 (m, 12H), 2.43-2.46 (m, 4H), 2.56-2.59 (m, 2H), 2.88-2.94 (m, 1H), 3.51-3.54 (m, 2H), 4.00-4.10 (m, 8H). LCMS: Rt: 0.080 min; MS m/z (ESI): 939.7 [M + H]⁺. |
| 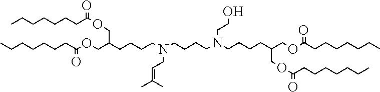 [SMILES: CCCCCCCC(=O)OCC(CCCCN(CC=C(C)C)CCCCN(CCO)CCCCC(COC(=O)CCCCCCC)COC(=O)CCCCCCC)COC(=O)CCCCCCC] | (m, 12H), 1.29-1.35 (m, 44H), 1.51-1.97(m, 20H), 2.28-2.31 (m, 10H), 2.50-2.90 (m, 10H), 3.49-4.02 (m, 3H), 4.03-4.05 (m, 8H), 5.30-5.31 (m, 1H). LCMS: Rt: 0.910 min; MS m/z (ESI): 965.7 [M + H]⁺. |
| Compound 203 | |
| 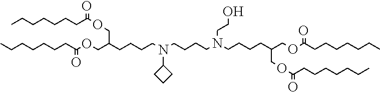 [SMILES: CCCCCCCC(=O)OCC(CCCCN(CCO)CCCCN(CCCCC(COC(=O)CCCCCCC)COC(=O)CCCCCCC)C1CCC1)COC(=O)CCCCCCC] | ¹H NMR (400 MHz, CDCl₃) δ: 0.86-0.90 (m, 12H), 1.26-1.36 (m, 43H), 1.59-1.70 (m, 18H), 1.94-2.12 (m, 5H), 2.28-2.32 (m, 8H), 2.39-2.81 (m, 10H), 3.53-3.60 (m, 2H), 4.02-4.07 (m, 8H). LCMS: Rt: 1.527 min; MS m/z (ESI): 951.7 [M + H]⁺. |
| Compound 219 | |

### 6.61 Example 61: Preparation of Compound 182

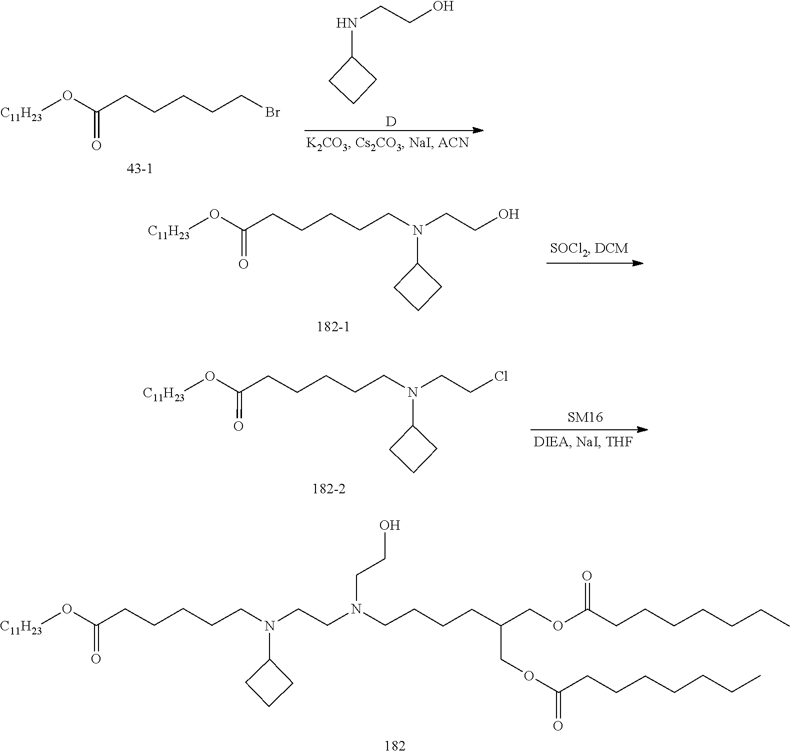

### Step 1: Preparation of Compound 182-1

To a solution of compound 43-1 (1.0 g, 2.86 mmol, 1.0 eq.) and compound D (495 mg, 4.29 mmol, 1.5 eq.) in ACN (30 mL) were added K₂CO₃ (1.2 g, 8.59 mmol, 3.0 eq.), Cs₂CO₃ (28 mg, 0.09 mmol, 0.03 eq.) and NaI (215 mg, 1.43 mmol, 0.5 eq.). The mixture was stirred for 16 hours at 70° C. The mixture was concentrated and purified by column chromatography on silica gel (MeOH/DCM=0/1-1/40) to give compound 182-1 (1.1 g, crude) as yellow oil. LCMS: Rt: 0.800 min; MS m/z (ESI): 384.4 [M+H]⁺.

### Step 2: Preparation of Compound 182-2

The mixture of compound 182-1 (300 mg, 0.78 mmol, 1.0 eq.) and SOCl₂ (279 mg, 2.35 mmol, 3.0 eq.) in DCM (6 mL) was stirred for overnight at 35° C. Quenched with water, extracted over EA, washed with brine, dried and concentrated to purified by FCC (MeOH/DCM=0% to 20%) to give compound 182-2 (190 mg, 60.42% yield) as light yellow oil.

### Step 3: Preparation of Compound 182

To a solution of compound 182-2 (190 mg, 0.47 mmol, 1.0 eq.) in THF (4 mL) were added compound SM16 (210 mg, 0.47 mmol, 1.0 eq.), DIEA (183 mg, 1.43 mmol, 3.0 eq.), NaI (35 mg, 0.24 mmol, 0.5 eq.) at room temperature. The mixture was stirred overnight at 70° C. The mixture was washed with water, brine, the organic layers were concentrated and purified by prep-HPLC to give compound 182 (57 mg, 15.55% yield) as colorless oil.

¹H NMR (400 MHz, CDCl₃) δ: 0.86-0.89 (m, 9H), 1.25-1.35 (m, 32H), 1.43-1.45 (m, 4H), 1.56-1.65 (m, 17H), 1.85-1.99 (m, 5H), 2.27-2.32 (m, 6H), 2.41-2.59 (m, 10H), 3.05-3.10 (m, 1H), 3.53-3.55 (m, 2H), 4.03-4.07 (m, 6H). LCMS: Rt: 1.230 min; MS m/z (ESI): 809.6 [M+H]⁺.

The following compounds were prepared in analogous fashion as Compound 182, using corresponding starting material.

| Compound | Characterization |
|---|---|
| 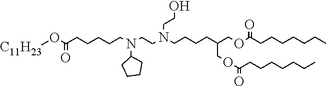 Compound 183 | ¹H NMR (400 MHz, CDCl₃) δ: 0.88-0.89 (m, 9H), 1.26-1.34 (m, 36H), 1.59-1.63 (m, 14H), 1.94-1.98 (m, 14H), 2.28-2.32 (m, 6H), 2.61-2.67 (m, 4H), 3.11-3.59 (m, 4H), 3.72-4.02 (m, 1H), 4.02-4.07 (m, 6H). LCMS: Rt: 1.185 min; MS m/z (ESI): 823.7 [M + H]⁺. |
| 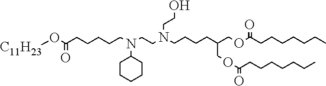 Compound 221 | ¹H NMR (400 MHz, CDCl₃) δ: 0.83-0.92 (m, 9H), 1.20-1.36 (m, 43H), 1.43-1.52 (m, 3H), 1.58-1.66 (m, 10H), 1.77-1.87 (m, 3H), 1.92-2.06 (m, 2H), 2.19-2.30 (m, 8H), 2.37- 2.74 (m, 10H), 3.44-3.65 (m, 2H), 4.00-4.13 (m, 6H). LCMS: Rt: 1.237 min; MS m/z (ESI): 837.7 [M + H]⁺. |

### 6.62 Example 62: Preparation of Compound 186

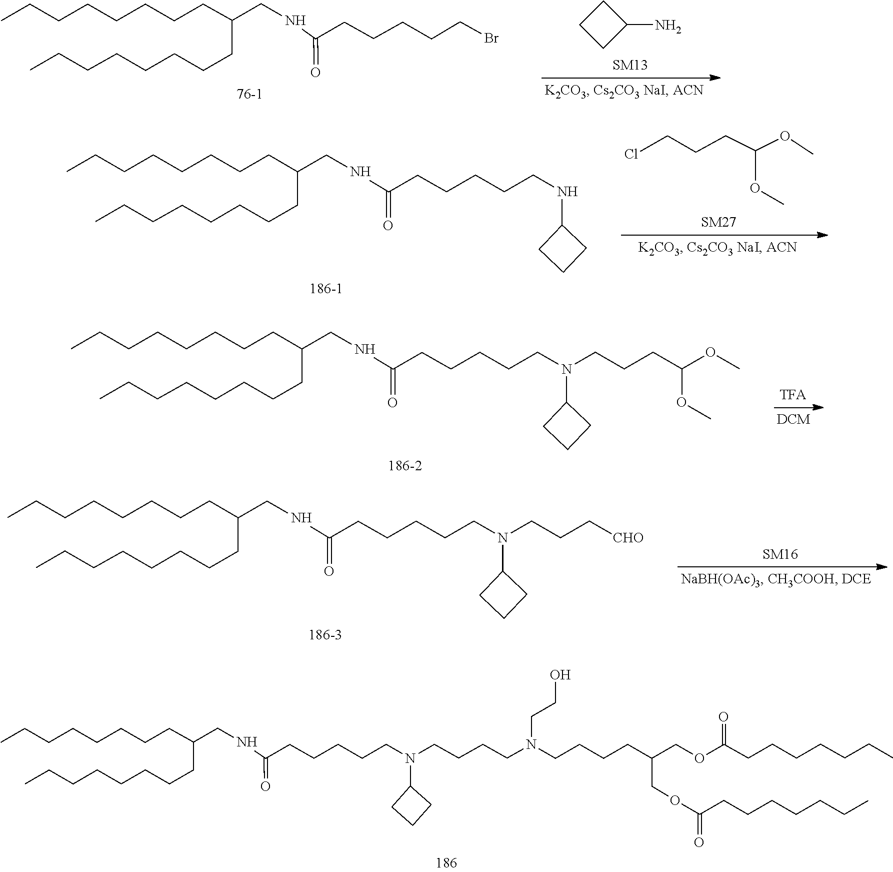

### Step 1: Preparation of Compound 186-1

To a solution of compound 76-1 (600 mg, 1.29 mmol, 1.0 eq.) in ACN (20 mL) were added compound SM13 (184 mg, 2.58 mmol, 2.0 eq.), K₂CO₃ (537 mg, 3.87 mmol, 3.0 eq.), Cs₂CO₃ (126 mg, 0.38 mmol, 0.3 eq.) and NaI (56 mg, 0.38 mmol, 0.3 eq.). The mixture was stirred at 80° C. for 10 hours. TLC showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 186-1 (350 mg, 61.8% yield) as colorless oil.

### Step 2: Preparation of Compound 186-2

To a solution of compound 186-1 (350 mg, 0.8 mmol, 1.0 eq.) and compound SM27 (360 mg, 2.4 mmol, 3.0 eq.) in ACN (25 mL) were added K₂CO₃ (332 mg, 2.4 mmol, 3.0 eq.), Cs₂CO₃ (78 mg, 0.24 mmol, 0.3 eq.) and NaI (34 mg, 0.24 mmol, 0.3 eq.). The mixture was stirred at 80° C. for 16 hours. TLC showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 186-2 (380 mg, 85.7% yield) as colorless oil.

### Step 3: Preparation of Compound 186-3

To a solution of compound 186-2 (380 mg, 0.68 mmol, 1.0 eq.) in DCM (20 mL) were added TFA (1 mL). The mixture was stirred at 25° C. for 10 hours. LCMS showed the reaction was completed. The reaction mixture was extracted with EA and Na₂CO₃ solution. The organic layer was washed with brine, dried over Na₂SO₄ and concentrated to give compound 186-3 (280 mg, 86.1% yield) as yellow oil.

### Step 4: Preparation of Compound 186

To a solution of compound 186-3 (280 mg, 0.55 mmol, 1.0 eq.) and compound SM16 (243 mg, 0.55 mmol, 1.0 eq.) in DCE (10 mL) were added two drops of CH₃COOH and stirred at 25° C. for 2 hours, then NaBH(OAc)₃ (233 mg, 1.1 mmol, 2.0 eq.) was added at 25° C. and stirred for 10 hours. LCMS showed the reaction was completed. The mixture was poured into water and extracted with EA. The combined organic layers were washed with brine, dried over Na₂SO₄ and concentrated. The residue was purified by prep-HPLC to give compound 186 (10 mg, 2.1% yield) as colorless oil.

¹H NMR (400 MHz, CDCl₃) δ: 0.86-0.89 (m, 12H), 1.26-1.61 (m, 54H), 1.63-1.71 (m, 18H), 2.22-2.31 (m, 8H), 2.76-2.92 (m, 6H), 2.91-3.17 (m, 2H), 3.44-3.80 (m, 6H), 3.81-4.07 (m, 4H), 5.70-5.83 (m, 1H). LCMS: Rt: 1.150 min; MS m/z (ESI): 934.7 [M+H]⁺.

### 6.63 Example 63: Preparation of Compound 187

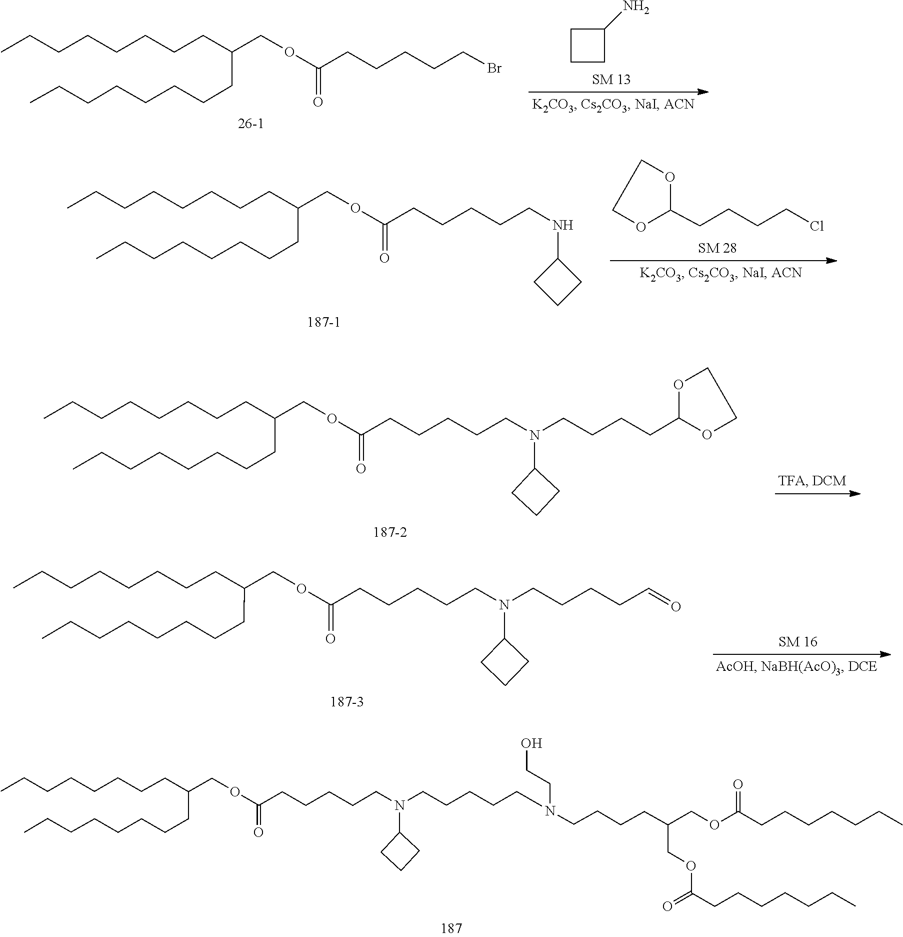

### Step 1: Preparation of Compound 187-1

To a solution of compound 26-1 (1.0 g, 2.23 mmol, 1.0 eq.) and compound SM13 (477 mg, 6.70 mmol, 3.0 eq.) in ACN (30 mL) were added K₂CO₃ (925 mg, 6.70 mmol, 3.0 eq.), Cs₂CO₃ (22 mg, 0.07 mmol, 0.03 eq.) and NaI (168 mg, 1.12 mmol, 0.5 eq.). The mixture was stirred for 16 hours at 80° C. The mixture was concentrated and purified by column chromatography on silica gel (MeOH/DCM=0/1-1/20) to give compound 187-1 (598 mg, 61.12% yield) as dark brown oil. LCMS: Rt: 0.970 min; MS m/z (ESI): 438.5 [M+H]⁺.

### Step 2: Preparation of Compound 187-2

To a solution of compound 187-1 (598 mg, 1.37 mmol, 1.0 eq.) and compound SM28 (247 mg, 1.50 mmol, 1.1 eq.) in ACN (12 mL) were added K₂CO₃ (565 mg, 4.10 mmol, 3.0 eq.), Cs₂CO₃ (13 mg, 0.04 mmol, 0.03 eq.) and NaI (102 mg, 0.68 mmol, 0.5 eq.). The mixture was stirred for 16 hours at 80° C. The mixture was concentrated and purified by column chromatography on silica gel (MeOH/DCM=0/1-1/60) to give compound 187-2 (485 mg, 62.74% yield) as light yellow oil. LCMS: Rt: 1.015 min; MS m/z (ESI): 566.6 [M+H]⁺.

### Step 3: Preparation of Compound 187-3

To the stirred solution of compound 187-2 (264 mg, 0.47 mmol, 1.0 eq.) in DCM (6 mL) was added TFA (6 mL, 80.4 mmol, 172.5 eq.) at room temperature. The mixture was stirred overnight at room temperature. Quenched with saturated sodium bicarbonate, extracted over EA, washed with brine, dried and concentrated to give compound 187-3 (221 mg, 90.77% yield) as dark brown oil. LCMS: Rt: 0.960 min; MS m/z (ESI): 522.5 [M+H]⁺.

### Step 4: Preparation of Compound 187

The mixture of compound 187-3 (110 mg, 0.21 mmol, 1.0 eq.), compound SM16 (94 mg, 0.21 mmol, 1.0 eq.) and two drops of acetic acid in DCE (6 mL) was stirred for 2 h at room temperature. NaBH(OAc)₃ (89 mg, 0.42 mmol, 2.0 eq.) was added to the above mixture, stirred overnight at room temperature. The mixture was quenched with water, extracted over DCM, the organic layers was concentrated and purified by prep-HPLC to give compound 187 (32 mg, 15.99% yield) as light yellow oil.

¹H NMR (400 MHz, CDCl₃) δ: 0.86-0.90 (m, 12H), 1.25-1.35 (m, 57H), 1.40-1.47 (m, 6H), 1.61-1.63 (m, 11H), 1.95-2.15 (m, 4H), 2.28-2.32 (m, 6H), 2.44-2.62 (m, 8H), 3.50-3.60 (m, 2H), 3.96-4.07 (m, 6H). LCMS: Rt: 1.190 min; MS m/z (ESI): 949.8 [M+H]⁺.

The following compounds were prepared in analogous fashion as Compound 187, using corresponding starting material.

| Compound | Characterization |
|---|---|
| 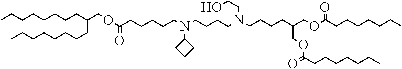 [SMILES: CCCCCCCCC(CCCCCCCC)COC(=O)CCCCCN(CCCCN(CCO)CCCCC(COC(=O)CCCCCCC)COC(=O)CCCCCCC)C1CCC1] Compound 220 | ¹H NMR (400 MHz, CDCl₃) δ: 0.86-0.90 (m, 12H), 1.27-1.37 (m, 51H), 1.60-1.70 (m, 14H), 1.87-2.02 (m, 3H), 2.08-2.18 (m, 3H), 2.29-2.34 (m, 7H), 2.56-2.87 (m, 11H), 3.46-3.48 (m, 1H), 3.70-3.76 (m, 2H), 3.96- 4.07 (m, 6H). LCMS: Rt: 1.035 min; MS m/z (ESI): 935.8 [M + H]⁺. |

### 6.64 Example 64: Preparation of Compound 188

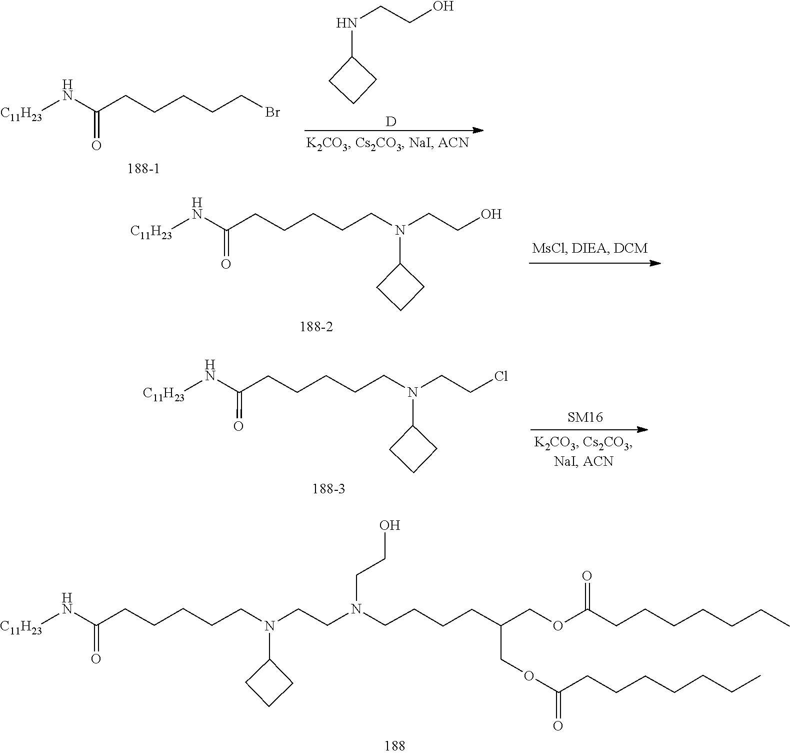

### Step 1: Preparation of Compound 188-2

To a solution of compound 188-1 (1.0 g, 2.87 mmol, 1.0 eq.) and compound D (660 mg, 5.74 mmol, 2.0 eq.) in ACN (30 mL) were added K₂CO₃ (1.2 g, 8.61 mmol, 3.0 eq.), Cs₂CO₃ (28 mg, 0.09 mmol, 0.03 eq.) and NaI (216 mg, 1.44 mmol, 0.5 eq.). The mixture was stirred for 16 hours at 80° C. The mixture was concentrated and purified by column chromatography on silica gel (MeOH/DCM=0/1-1/30) to give compound 188-2 (632 mg, 57.55% yield) as yellow oil. LCMS: Rt: 0.770 min; MS m/z (ESI): 383.4 [M+H]⁺.

### Step 2: Preparation of Compound 188-3

To the stirred solution of compound 188-2 (341 mg, 0.89 mmol, 1.0 eq.) and DIEA (172 mg, 1.34 mmol, 1.5 eq.) in DCM (6 mL) was added MsCl (112 mg, 0.98 mmol, 1.1 eq.) dropwise in ice bath. The mixture was stirred for an hour at room temperature. Quenched with water, extracted over EA, washed with brine, dried and concentrated to purified by FCC (MeOH/DCM=0% to 1.67%) to give compound 188-3 (141 mg, 34.99% yield) as yellow oil. LCMS: Rt: 0.790 min; MS m/z (ESI): 401.4 [M+H]⁺.

### Step 3: Preparation of Compound 188

The mixture of compound 188-3 (120 mg, 0.30 mmol, 1.0 eq.), K₂CO₃ (124 mg, 0.90 mmol, 3.0 eq.), Cs₂CO₃ (3 mg, 0.01 mmol, 0.03 eq.), compound SM16 (159 mg, 0.36 mmol, 1.2 eq.) and NaI (22 mg, 0.15 mmol, 0.5 eq.) in ACN (5 mL) was stirred for 48 h at 90° C. The mixture was washed with water, brine, the organic layers were concentrated and purified by prep-HPLC to give compound 188 (32 mg, 13.23% yield) as colorless oil.

¹H NMR (400 MHz, CDCl₃) δ: 0.86-0.90 (m, 9H), 1.26-1.35 (m, 41H), 1.47-1.49 (m, 6H), 1.59-1.65 (m, 8H), 1.95-2.02 (m, 8H), 2.15-2.20 (m, 2H), 2.28-2.32 (m, 5H), 2.50-2.66 (m, 6H), 3.20-3.25 (m, 2H), 3.55-3.61 (m, 2H), 4.00-4.10 (m, 4H). LCMS: Rt: 1.050 min; MS m/z (ESI): 808.7 [M+H]⁺.

The following compounds were prepared in analogous fashion as Compound 188, using corresponding starting material.

| Compound | Characterization |
|---|---|
| 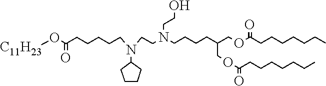 Compound 204 | ¹H NMR (400 MHz, CDCl₃) δ: 0.86-0.90 (m, 9H), 1.25-1.35 (m, 43H), 1.47-1.52 (m, 4H), 1.59-1.72 (m, 9H), 1.81-1.85 (m, 3H), 1.95-2.05 (m, 4H), 2.24-2.32 (m, 6H), 2.56- 2.60 (m, 2H), 2.67-2.72 (m, 2H), 2.99-3.11 (m, 5H), 3.20-3.25 (m, 2H), 3.64-3.72 (m, 2H), 4.00-4.10 (m, 4H). LCMS: Rt: 1.040 min; MS m/z (ESI): 822.7 [M + H]⁺. |
| 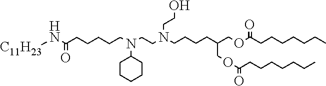 Compound 222 | ¹H NMR (400 MHz, CDCl₃) δ: 0.86-0.89 (m, 9H), 1.17-1.36 (m, 41H), 1.42-1.50 (m, 5H), 1.60-1.78 (m, 14H), 1.95-2.00 (m, 2H), 2.14-2.18 (m, 2H), 2.28-2.32 (m, 4H), 2.41- 2.60 (m, 11H), 3.20-3.25 (m, 2H), 3.52-3.54 (m, 2H), 4.01-4.10 (m, 4H), 5.75 (s, 1H). LCMS: Rt: 1.044 min; MS m/z (ESI): 836.1 [M + H]⁺. |

### 6.65 Example 65: Preparation of Compound 190

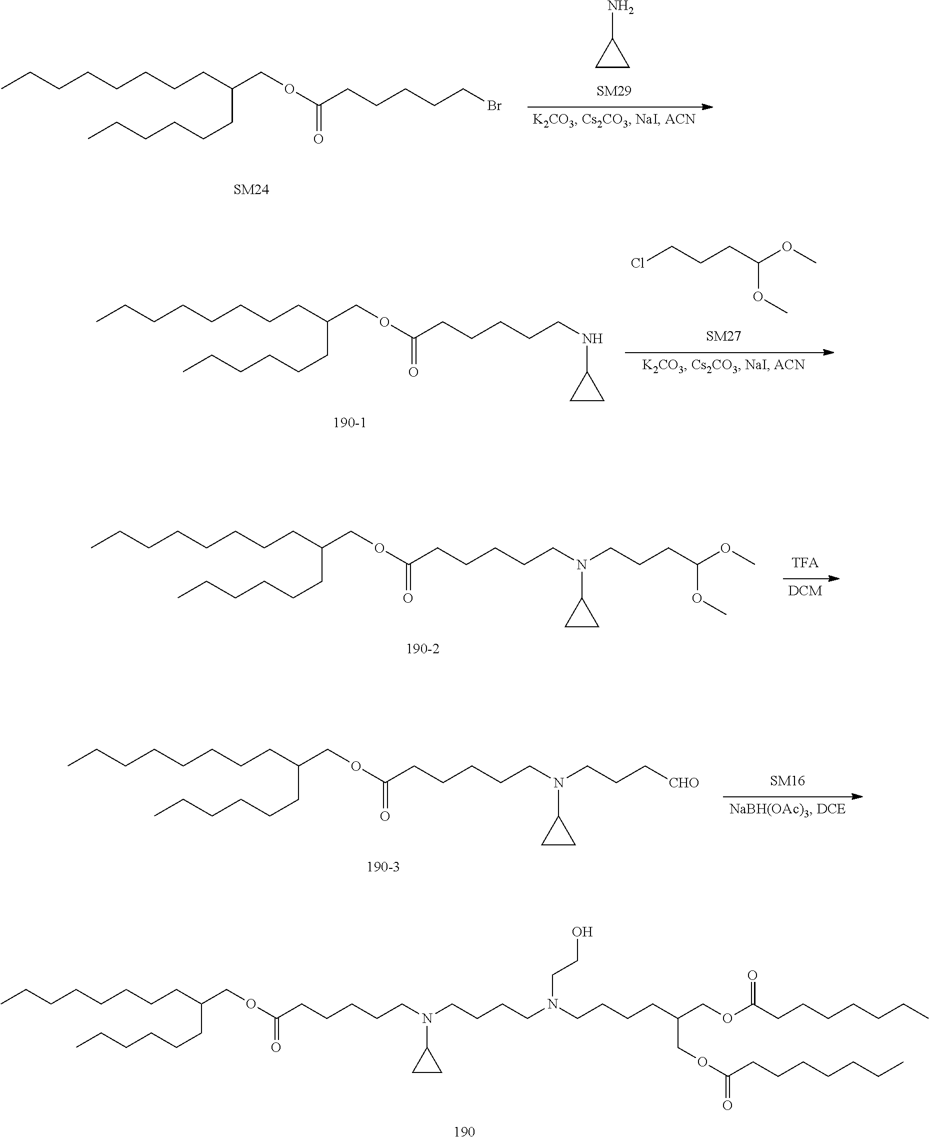

### Step 1: Preparation of Compound 190-1

To a solution of compound SM24 (1 g, 2.3 mmol, 1.0 eq.) in ACN (25 mL) were added compound SM29 (0.37 g, 6.9 mmol, 3.0 eq.), K₂CO₃ (0.98 g, 6.9 mmol, 3.0 eq.), Cs₂CO₃ (0.23 g, 0.69 mmol, 0.3 eq.) and NaI (0.1 g, 0.69 mmol, 0.3 eq.). The mixture was stirred at 80° C. for 10 hours. TLC showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 190-1 (500 mg, 53% yield) as colorless oil.

### Step 2: Preparation of Compound 190-2

To a solution of compound 190-1 (500 mg, 1.26 mmol, 1.0 eq.) and compound SM27 (578 mg, 3.78 mmol, 3.0 eq.) in ACN (15 mL) were added K₂CO₃ (523 mg, 3.78 mmol, 3.0 eq.), Cs₂CO₃ (123 mg, 0.38 mmol, 0.3 eq.) and NaI (54 mg, 0.38 mmol, 0.3 eq.). The mixture was stirred at 80° C. for 16 hours. TLC showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 190-2 (523 mg, 80.9% yield) as colorless oil.

### Step 3: Preparation of Compound 190-3

To a solution of compound 190-2 (523 mg, 1.08 mmol, 1.0 eq.) in DCM (15 mL) were added TFA (1 mL). The mixture was stirred at 25° C. for 10 hours. LCMS showed the reaction was completed. The reaction mixture was extracted with EA and Na₂CO₃ solution. The organic layer were washed with brine, dried over Na₂SO₄ and concentrated to give compound 190-3 (380 mg, 79.8% yield) as colorless oil.

### Step 4: Preparation of Compound 190

To a solution of compound 190-3 (350 mg, 0.75 mmol, 1.0 eq.) and compound SM16 (332 mg, 0.75 mmol, 1.0 eq.) in DCE (10 mL) were added two drops of CH₃COOH and stirred at 25° C. for 2 hours, then NaBH(OAc)₃ (310 mg, 1.5 mmol, 2.0 eq.) was added at 25° C. and stirred for 10 hours. LCMS showed the reaction was completed. The mixture was poured into water and extracted with EA. The combined organic layers were washed with brine, dried over Na₂SO₄ and concentrated. The residue was purified by prep-HPLC to give compound 190 (70 mg, 10.4% yield) as colorless oil.

¹H NMR (400 MHz, CDCl₃) δ: 0.51-0.86 (m, 12H), 1.28-1.39 (m, 48H), 1.60-1.68 (m, 24H), 2.28-2.31 (m, 8H), 2.32-2.69 (m, 6H), 3.96-4.06 (m, 6H). LCMS: Rt: 1.150 min; MS m/z (ESI): 893.7 [M+H]⁺.

The following compounds were prepared in analogous fashion as Compound 190, using corresponding starting material.

| Compound | Characterization |
|---|---|
| 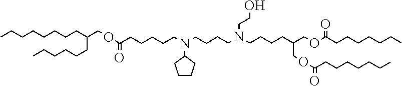 [SMILES: CCCCCCCCC(CCCCCC)COC(=O)CCCCCN(CCCCN(CCO)CCCCC(COC(=O)CCCCCCC)COC(=O)CCCCCCC)C1CCCC1] | ¹H NMR (400 MHz, CDCl₃) δ: 0.86-0.89 (m, 12H), 1.27-1.38 (m, 48H), 1.60-2.01 (m, 24H), 2.29-2.33 (m, 8H), 2.49-3.13 (m, 9H), 3.69-3.96 (m, 1H), 3.97-4.07 (m, 6H). LCMS: Rt: 1.150 min; MS m/z (ESI): 921.7 [M + H]⁺. |
| Compound 191 | |
| 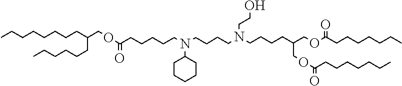 [SMILES: CCCCCCCCC(CCCCCC)COC(=O)CCCCCN(CCCCN(CCO)CCCCC(COC(=O)CCCCCCC)COC(=O)CCCCCCC)C1CCCCC1] Compound 192 | ¹H NMR (400 MHz, CDCl₃) δ: 0.86-0.90 (m, 12H), 1.22-1.41 (m, 56H), 1.61-1.74 (m, 12H), 1.90-2.00 (m, 9H), 2.19 (s, 2H), 2.29-2.35 (m, 6H), 2.91-3.03 (m, 4H), 3.16 (s, 1H), 3.55-3.74 (m, 2H), 3.96-4.10 (m, 6H). LCMS: Rt: 1.350 min; MS m/z (ESI): 935.7 [M + H]⁺. |
| 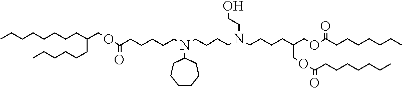 [SMILES: CCCCCCCCC(CCCCCC)COC(=O)CCCCCN(CCCCN(CCO)CCCCC(COC(=O)CCCCCCC)COC(=O)CCCCCCC)C1CCCCCC1] | ¹H NMR (400 MHz, CDCl₃) δ: 0.80-0.90 (m, 12H), 1.20-1.48 (m, 47H), 1.49-1.80 (m, 24H), 1.93-2.22 (m, 4H), 2.28-2.34 (m, 7H), 2.46-2.61 (m, 10H), 3.52-3.64 (m, 2H), 3.91-4.14 (m, 6H). LCMS: Rt: 0.985 min; MS m/z (ESI): 949.8 [M + H]⁺. |
| Compound 193 | |
| 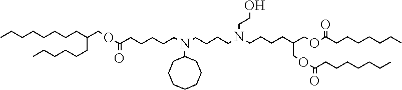 [SMILES: CCCCCCCCC(CCCCCC)COC(=O)CCCCCN(CCCCN(CCO)CCCCC(COC(=O)CCCCCCC)COC(=O)CCCCCCC)C1CCCCCCC1] Compound 194 | ¹H NMR (400 MHz, CDCl₃) δ: 0.86-0.90 (m, 12H), 1.27-1.44 (m, 62H), 1.60-1.63 (m, 15H), 1.96-2.00 (m, 2H), 2.28-2.32 (m, 8H), 2.44-2.49 (m, 3H), 2.56-2.61 (m, 2H), 2.69-2.74 (m, 1H), 2.87-2.91 (m, 1H), 3.55 (s, 2H), 3.96-4.12 (m, 6H). LCMS: Rt: 1.380 min; MS m/z (ESI): 963.8 [M + H]⁺. |
| 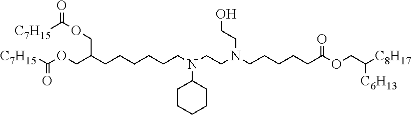 | ¹H NMR (400 MHz, CDCl₃) δ: 0.86-0.90 (m, 12H), 1.11-1.33 (m, 58H), 1.48-1.52 (m, 3H), 1.60-1.64 (m, 9H), 1.73-1.85 (m, 3H), 1.93-2.01 (m, 1H), 2.28-2.33 (m, 6H), 2.38-2.66 (m, 10H), 3.52-3.62 (m, 2H), 3.96-4.07 (m, 6H). LCMS: Rt: 1.450 min; MS m/z (ESI): 935.8 [M + H]⁺. |
| Compound 252 | |
| 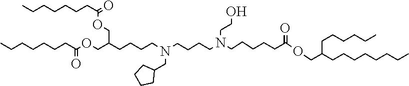 [SMILES: CCCCCCCCC(CCCCCC)COC(=O)CCCCCN(CCO)CCCCN(CCCCC(COC(=O)CCCCCCC)COC(=O)CCCCCCC)CC1CCCC1] | ¹H NMR (400 MHz, CDCl₃) δ: 0.86-0.90 (m, 12H), 1.14-1.40 (m, 54H), 1.59-1.65 (m, 15H), 1.96-2.07 (m, 2H), 2.15-2.39 (m, 9H), 2.49 -2.64 (m, 10H), 3.48-3.64 (m, 2H), 3.93-4.12 (m, 6H). LCMS: Rt: 1.015 min; MS m/z (ESI): 935.7 [M + H]⁺. |
| Compound 253 | |
| 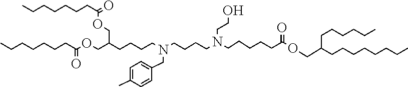 [SMILES: CCCCCCCCC(CCCCCC)COC(=O)CCCCCN(CCO)CCCCN(CCCCC(COC(=O)CCCCCCC)COC(=O)CCCCCCC)Cc1ccc(C)cc1] | ¹H NMR (400 MHz, CDCl₃) δ: 0.86-0.89 (m, 12H), 1.28-1.41 (m, 44H), 1.42-1.61 (m, 16H), 1.69-2.33 (m, 16H), 2.39-4.02 (m, 8H), 4.03-4.08 (m, 8H), 7.23-7.26 (m, 4H). LCMS: Rt: 1.225 min; MS m/z (ESI): 957.7 [M + H]⁺. |
| Compound 254 | |

### 6.66 Example 66: Preparation of Compound 195

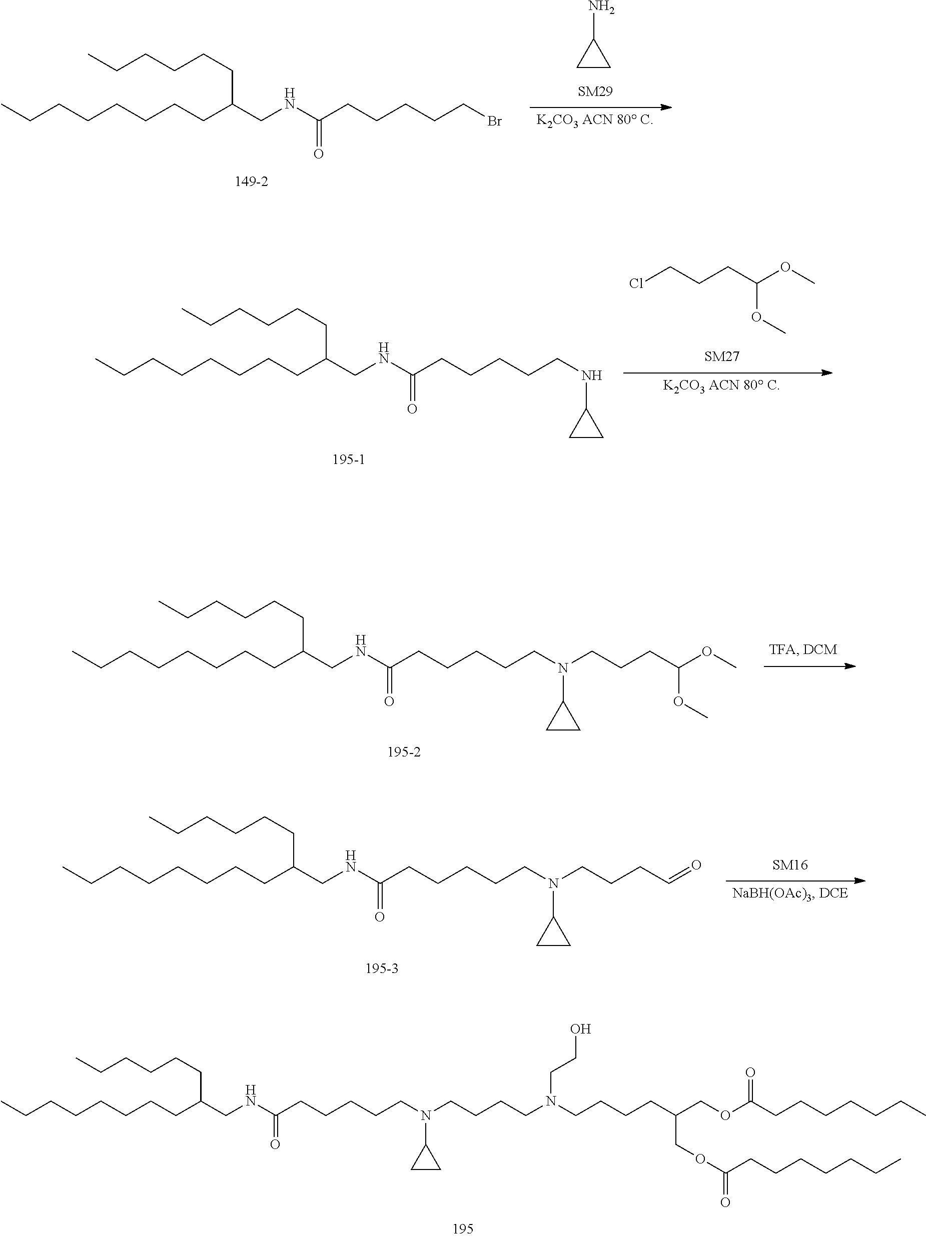

### Step 1: Preparation of Compound 195-1

A solution of compound 149-2 (0.6 g, 1.43 mmol, 1.0 eq.), compound SM29 (250 mg, 4.3 mmol, 3.0 eq.), K₂CO₃ (590 mg, 4.3 mmol, 3.0 eq.), Cs₂CO₃ (140 mg, 0.43 mmol, 0.3 eq.) and NaI (65 mg, 0.43 mmol, 0.3 eq.) in ACN (10 mL) was stirred at 80° C. for overnight. TLC showed the reaction was completed. The mixture was concentrated and purified by FCC to give compound 195-1 (300 mg, 53.15% yield) as yellow oil.

### Step 2: Preparation of Compound 195-2

To a solution of compound 195-1 (300 mg, 0.76 mmol, 1.0 eq.), compound SM27 (350 mg, 2.28 mmol, 3.0 eq.), K₂CO₃ (320 mg, 2.28 mmol, 3.0 eq.), Cs₂CO₃ (75 mg, 0.23 mmol, 0.3 eq.) and NaI (35 mg, 0.23 mmol, 0.3 eq.) in ACN (10 mL) was stirred at 80° C. for 40 h. TLC showed the reaction was completed. The mixture was concentrated and purified by FCC to give compound 195-2 (300 mg, 77.28% yield) as yellow oil.

### Step 3: Preparation of Compound 195-3

The mixture of compound 195-2 (300 mg, 0.59 mmol, 1.0 eq.) and TFA (0.5 mL) in DCM (10 mL) was stirred at RT for overnight. TLC showed the reaction was completed. The mixture was diluted with DCM and washed with water and bring, dried, concentrated, to give compound 195-3 (280 mg, crude) as yellow oil, which was used for the next step without further purification.

### Step 4: Preparation of Compound 195

To a solution of compound 195-3 (280 mg, 0.59 mmol, 1.0 eq.), compound SM16 (310 mg, 0.71 mmol, 1.2 eq.) in DCE (10 mL) was stirred at rt for overnight. The NaBH(AcO)₃ (250 mg, 1.2 mmol, 2.0 eq.) was added. After stirring for 24 h, LCMS showed the reaction was completed. The mixture was concentrated and purified by prep-HPLC to give compound 195 (110 mg, 20.89% yield) as yellow oil.

¹H NMR (400 MHz, CDCl₃) δ: 0.36-0.45 (m, 4H), 0.86-0.90 (m, 12H), 1.26-1.35 (m, 46H), 1.40-1.55 (m, 8H), 0.60-1.77 (m, 9H), 1.97-2.00 (m, 1H), 2.15-2.19 (m, 2H), 2.29-2.32 (m, 4H), 2.43-2.59 (m, 10H), 3.16-3.19 (m, 2H), 3.51-3.54 (m, 2H), 4.00-4.10 (m, 4H), 5.50 (s, 1H). LCMS: Rt: 0.080 min; MS m/z (ESI): 892.6 [M+H]⁺.

The following compounds were prepared in analogous fashion as Compound 195, using corresponding starting material.

| Compound | Characterization |
|---|---|
| 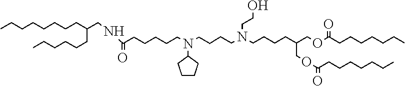 [SMILES: CCCCCCCCC(CCCCCC)CNC(=O)CCCCCN(CCCCN(CCO)CCCCC(COC(=O)CCCCCCC)COC(=O)CCCCCCC)C1CCCC1] Compound 196 | ¹H NMR (400 MHz, CDCl₃) δ: 0.86-0.90 (m, 12H), 1.26-1.34 (m, 48H), 1.35-1.88 (m, 22H), 1.96-1.99 (m, 1H), 2.17-2.20 (m, 2H), 2.28-2.32 (m, 4H), 2.44-2.61 (m, 9H), 2.86-3.12 (m, 2H), 3.16-3.19 (m, 2H), 3.53- 3.55 (m, 2H), 4.00-4.10 (m, 4H), 5.51-5.67 (m, 1H). LCMS: Rt: 1.200 min; MS m/z (ESI): 920.8 [M + H]⁺. |
| 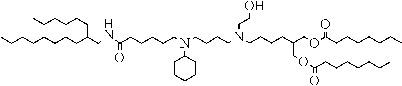 [SMILES: CCCCCCCCC(CCCCCC)CNC(=O)CCCCCN(CCCCN(CCO)CCCCC(COC(=O)CCCCCCC)COC(=O)CCCCCCC)C1CCCCC1] Compound 197 | ¹H NMR (400 MHz, CDCl₃) δ: 0.86-0.90 (m, 12H), 1.18-1.46 (m, 66H), 1.63-1.81 (m, 6H), 1.95-2.03 (m, 1H), 2.14-2.21 (m, 2H), 2.28-2.30 (m, 4H), 2.32-2.35 (m, 9H), 2.54-2.57 (m, 2H), 3.14-3.21 (m, 2H), 3.49- 3.56 (m, 2H), 4.00-4.14 (m, 4H), 5.53-5.61 (m, 1H). LCMS: Rt: 0.920 min; MS m/z (ESI): 934.7 [M + H]⁺. |
| 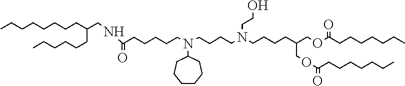 [SMILES: CCCCCCCCC(CCCCCC)CNC(=O)CCCCCN(CCCCN(CCO)CCCCC(COC(=O)CCCCCCC)COC(=O)CCCCCCC)C1CCCCCC1] | ¹H NMR (400 MHz, CDCl₃) δ: 0.86-0.90 (m, 12H), 1.26-1.76 (m, 74H), 1.94-2.61 (m, 18H), 3.16-3.19 (m, 2H), 3.51-3.54 (m, 2H), 4.03-4.07 (m, 4H), 5.59-5.62 (m, 1H). LCMS: Rt: 1.110 min; MS m/z (ESI): 948.8 [M + H]⁺. |
| Compound 198 | |
| 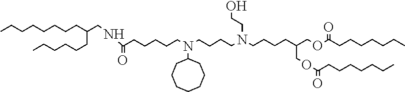 [SMILES: CCCCCCCCC(CCCCCC)CNC(=O)CCCCCN(CCCCN(CCO)CCCCC(COC(=O)CCCCCCC)COC(=O)CCCCCCC)C1CCCCCCC1] Compound 199 | ¹H NMR (400 MHz, CDCl₃) δ: 0.83-0.99 (m, 12H), 1.26-1.44 (m, 61H), 1.58-1.78 (m, 16H), 1.93-1.98 (m, 2H), 2.15-2.23 (m, 2H), 2.26-2.44 (m, 8H), 2.47-2.54 (m, 4H), 2.57-2.63 (m, 2H), 3.13-3.21 (m, 2H), 3.50- 3.58 (m, 2H), 3.98-4.14 (m, 4H). LCMS: Rt: 0.945 min; MS m/z (ESI): 962.8 [M + H]⁺. |
| 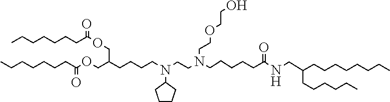 [SMILES: CCCCCCCCC(CCCCCC)CNC(=O)CCCCCN(CCOCCO)CCN(CCCCC(COC(=O)CCCCCCC)COC(=O)CCCCCCC)C1CCCC1] Compound 205 | ¹H NMR (400 MHz, CDCl₃) δ: 0.86-0.90 (m, 12H), 1.26-1.38 (m, 47H), 1.47-1.51 (m, 6H), 1.59-1.65 (m, 12H), 1.76-1.79 (m, 2H), 1.97-2.02 (m, 2H), 2.15-2.20 (m, 2H), 2.28-2.32 (m, 4H), 2.45-2.49 (m, 4H), 2.61- 2.66 (m, 5H), 2.96-3.00 (m, 1H), 3.16-3.19 (m, 2H), 3.55-3.68 (m, 6H), 4.00-4.10 (m, 4H). LCMS: Rt: 1.430 min; MS m/z (ESI): 936.7 [M + H]⁺. |
| 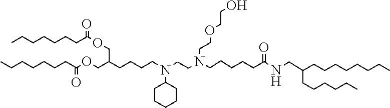 [SMILES: CCCCCCCCC(CCCCCC)CNC(=O)CCCCCN(CCOCCO)CCN(CCCCC(COC(=O)CCCCCCC)COC(=O)CCCCCCC)C1CCCCC1] | ¹H NMR (400 MHz, CDCl₃) δ: 0.81-0.90 (m, 12H), 1.16-1.42 (m, 54H), 1.45-1.98 (m, 16H), 2.19-2.22 (m, 2H), 2.27-2.36 (m, 5H), 2.45-2.73 (m, 8H), 3.13-3.23 (m, 3H), 3.55-3.73 (m, 6H), 4.00-4.11 (m, 4H), 5.27- 5.33 (m, 1H). LCMS: Rt: 1.095 min; MS m/z (ESI): 950.8 [M + H]⁺. |
| Compound 206 | |
| 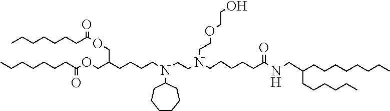 [SMILES: CCCCCCCCC(CCCCCC)CNC(=O)CCCCCN(CCOCCO)CCN(CCCCC(COC(=O)CCCCCCC)COC(=O)CCCCCCC)C1CCCCCC1] | ¹H NMR (400 MHz, CDCl₃) δ: 0.88-0.89 (m, 12H), 1.26-1.39 (m, 47H), 1.57-1.61 (m, 22H), 2.29-2.32 (m, 12H), 3.03-3.39 (m, 10H), 3.57-3.76 (m, 6H), 3.97-4.13 (m, 4H). LCMS: Rt: 1.350 min; MS m/z (ESI): 964.8 [M + H]⁺. |
| Compound 207 | |
| 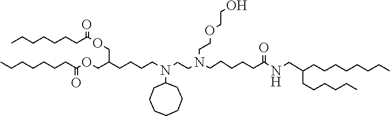 [SMILES: CCCCCCCCC(CCCCCC)CNC(=O)CCCCCN(CCOCCO)CCN(CCCCC(COC(=O)CCCCCCC)COC(=O)CCCCCCC)C1CCCCCCC1] | ¹H NMR (400 MHz, CDCl₃) δ: 0.88-0.90 (m, 12H), 1.26-1.40 (m, 49H), 1.59-1.61 (m, 18H), 1.62-2.28 (m, 9H), 2.30-2.76 (m, 8H), 2.92-3.17 (m, 8H), 3.42-3.77 (m, 6H), 3.99-4.15 (m, 4H), 5.87-5.92 (m, 1H). LCMS: Rt: 1.310 min; MS m/z (ESI): 978.8 [M + H]⁺. |
| Compound 208 | |
| 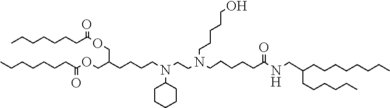 [SMILES: CCCCCCCCC(CCCCCC)CNC(=O)CCCCCN(CCCCCO)CCN(CCCCC(COC(=O)CCCCCCC)COC(=O)CCCCCCC)C1CCCCC1] Compound 209 | ¹H NMR (400 MHz, CDCl₃) δ: 0.86-0.90 (m, 12H), 1.15-1.39 (m, 54H), 1.47-1.61 (m, 5H), 1.67-1.76 (m, 9H), 1.79 (s, 4H), 1.95-1.99 (m, 4H), 2.15-2.19 (m, 2H), 2.28- 2.32 (m, 4H), 2.48 (d, J = 30.4 Hz, 10H), 3.16-3.19 (m, 2H), 3.63-3.66 (m, 2H), 4.00- 4.10 (m, 4H), 5.48 (s, 1H). LCMS: Rt: 0.080 min; MS m/z (ESI): 948.7 [M + H]⁺. |
| 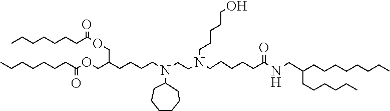 | ¹H NMR (400 MHz, CDCl₃) δ: 0.79-0.82 (m, 12H), 1.19-1.36 (m, 50H), 1.43-1.49 (m, 20H), 1.50-1.54 (m, 8H), 1.76-2.24 (m, 10H), 2.25-2.52 (m, 6H), 2.68-2.93 (m, 5H), 3.52-3.59 (m, 2H), 3.96-4.00 (m, 4H). LCMS: Rt: 1.540 min; MS m/z (ESI): 976.8 [M + H]⁺. |
| Compound 210 | |
| 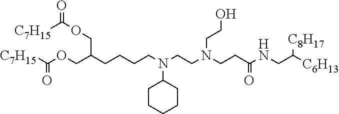 | ¹H NMR (400 MHz, CDCl₃) δ: 0.86-0.90 (m, 12H), 1.08-1.99 (m, 62H), 2.28-2.85 (m, 17H), 3.15-3.18 (m, 2H), 3.59-3.61 (m, 2H), 4.00-4.50 (m, 5H), 6.55-6.72 (m, 1H). LCMS: Rt: 1.167 min; MS m/z (ESI): 864.7 [M + H]⁺. |
| Compound 211 | |
| 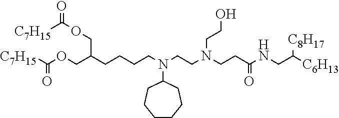 | ¹H NMR (400 MHz, CDCl₃) δ: 0.86-0.90 (m, 12H), 1.26-1.99 (m, 68H), 2.28-2.87 (m, 15H), 3.15-3.18 (m, 2H), 3.51-3.67 (m, 2H), 4.00-4.10 (m, 4H). LCMS: Rt: 1.190 min; MS m/z (ESI): 878.8 [M + H]⁺. |
| Compound 212 | |
| 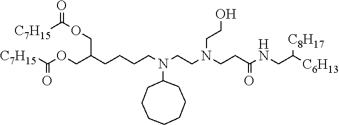 | ¹H NMR (400 MHz, CDCl₃) δ: 0.86-0.90 (m, 12H), 1.26-1.71 (m, 66H), 1.95-2.02 (m, 1H), 2.28-2.44 (m, 10H), 2.57-2.65 (m, 4H), 2.78-2.84 (m, 3H), 3.15-3.17 (m, 2H), 3.58-3.64 (m, 2H), 4.03-4.08 (m, 4H), 6.50- 6.58 (m, 1H). LCMS: Rt: 1.337 min; MS m/z (ESI): 892.6 [M + H]⁺. |
| Compound 213 | |
| 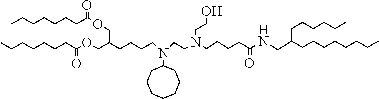 [SMILES: CCCCCCCCC(CCCCCC)CNC(=O)CCCCN(CCO)CCN(CCCCC(COC(=O)CCCCCCC)COC(=O)CCCCCCC)C1CCCCCCC1] Compound 214 | ¹H NMR (400 MHz, CDCl₃) δ: 0.86-0.90 (m, 12H), 1.26-1.30 (m, 40H), 1.42-1.49 (m, 11H), 1.59-1.63 (m, 16H), 1.70-1.72 (m, 4H), 1.96-2.03 (m, 1H), 2.16-2.20 (m, 2H), 2.27-2.41 (m, 8H), 2.49-2.53 (m, 4H), 2.57-2.59 (m, 2H), 2.75-2.80 (m, 1H), 3.16- 3.20 (m, 2H), 3.51-3.54 (m, 2H), 4.00-4.10 (m, 4H). LCMS: Rt:1.470 min; MS m/z (ESI): 920.7 [M + H]⁺. |
| 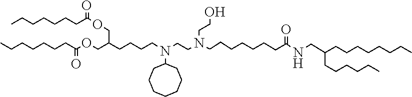 [SMILES: CCCCCCCCC(CCCCCC)CNC(=O)CCCCCCCN(CCO)CCN(CCCCC(COC(=O)CCCCCCC)COC(=O)CCCCCCC)C1CCCCCCC1] Compound 215 | ¹H NMR (400 MHz, CDCl₃) δ: 0.86-0.90 (m, 12H), 1.26-1.44 (m, 70H), 1.63-1.76 (m, 6H), 1.94-2.06 (m, 1H), 2.14-2.18 (m, 2H), 2.26-2.59 (m, 14H), 2.77-2.83 (m, 1H), 3.16-3.21 (m, 2H), 3.51-3.54 (m, 2H), 4.00- 4.16 (m, 4H), 5.45-5.50 (m, 1H). LCMS: Rt: 1.230 min; MS m/z (ESI): 962.8 [M + H]⁺. |
| 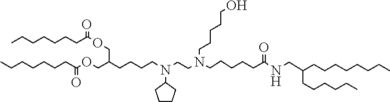 [SMILES: CCCCCCCCC(CCCCCC)CNC(=O)CCCCCN(CCCCCO)CCN(CCCCC(COC(=O)CCCCCCC)COC(=O)CCCCCCC)C1CCCC1] Compound 224 | ¹H NMR (400 MHz, CDCl₃) δ: 0.81-0.90 (m, 12H), 1.26-1.37 (m, 49H), 1.41-1.52 (m, 9H), 1.56-1.66 (m, 10H), 1.77 (s, 2H), 1.95-2.04 (m, 4H), 2.15-2.19 (m, 2H), 2.28- 2.32 (m, 4H), 2.43-2.59 (m, 9H), 2.99-3.03 (m, 1H), 3.16-3.19 (m, 2H), 3.62-3.66 (m, 2H), 4.00-4.10 (m, 4H), 5.49 (s, 1H). LCMS: Rt: 0.093 min; MS m/z (ESI): 934.7 [M + H]⁺. |
| 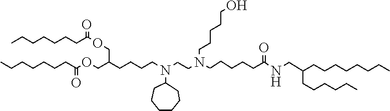 [SMILES: CCCCCCCCC(CCCCCC)CNC(=O)CCCCCN(CCCCCO)CCN(CCCCC(COC(=O)CCCCCCC)COC(=O)CCCCCCC)C1CCCCCC1] | ¹H NMR (400 MHz, CDCl₃) δ: 0.88-0.89 (m, 12H), 1.26-1.45 (m, 48H), 1.49-1.61 (m, 21H), 1.63-1.98 (m, 20H), 1.99-2.97 (m, 6H), 3.17-3.65 (m, 2H), 3.66-4.03 (m, 2H), 4.04-4.07 (m, 4H). LCMS: Rt: 1.500 min; MS m/z (ESI): 962.8 [M + H]⁺. |
| Compound 225 | |
| 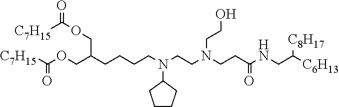 | ¹H NMR (400 MHz, CDCl₃) δ: 0.86-0.90 (m, 12H), 1.26-2.01 (m, 61H), 2.29-2.37 (m, 6H), 2.53-2.65 (m, 8H), 2.83-3.18 (m, 5H), 3.59-3.62 (m, 2H), 4.03-4.08 (m, 4H), 6.65-6.72 (m, 1H). LCMS: Rt: 1.200 min; MS m/z (ESI): 850.7 [M + H]⁺. |
| Compound 226 | |
| 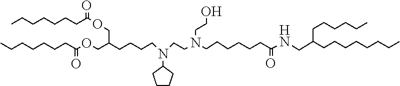 Compound 227 | ¹H NMR (400 MHz, CDCl₃) δ: 0.86-0.89 (m, 12H), 1.26-1.39 (m, 47H), 1.59-1.61 (m, 16H), 1.96-2.02 (m, 3H), 2.19-2.33 (m, 6H), 2.59-2.70 (m, 5H), 2.97-3.18 (m, 7H), 3.48-3.69 (m, 3H), 4.00-4.10 (m, 4H). LCMS: Rt: 1.265 min; MS m/z (ESI): 878.7 [M + H]⁺. |
| 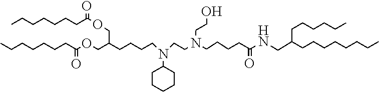 [SMILES: CCCCCCCCC(CCCCCC)CNC(=O)CCCCN(CCO)CCN(CCCCC(COC(=O)CCCCCCC)COC(=O)CCCCCCC)C1CCCCC1] Compound 228 | ¹H NMR (400 MHz, CDCl₃) δ: 0.86-0.90 (m, 12H), 1.26-1.38 (m, 52H), 1.49-1.69 (m, 11H), 1.85-2.02 (m, 4H), 2.18-2.32 (m, 7H), 2.48-2.74 (m, 6H), 2.92-3.17 (m, 7H), 3.50-3.74 (m, 2H), 4.03-4.07 (m, 4H). LCMS: Rt: 1.215 min; MS m/z (ESI): 892.7 [M + H]⁺. |
| 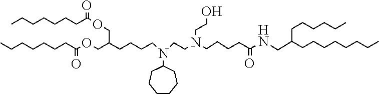 [SMILES: CCCCCCCCC(CCCCCC)CNC(=O)CCCCN(CCO)CCN(CCCCC(COC(=O)CCCCCCC)COC(=O)CCCCCCC)C1CCCCCC1] Compound 229 | ¹H NMR (400 MHz, CDCl₃) δ: 0.86-0.90 (m, 12H), 1.26-1.30 (m, 46H), 1.39 (s, 4H), 1.53-1.70 (m, 16H), 1.78-2.01 (m, 4H), 2.21-2.25 (m, 3H), 2.28-2.32 (m, 4H), 2.61- 2.73 (m, 4H), 2.94-3.04 (m, 5H), 3.15-3.18 (m, 2H), 3.42 (s, 1H), 3.69 (s, 2H), 3.99- 4.11 (m, 4H). LCMS: Rt:1.450 min; MS m/z (ESI): 906.8 [M + H]⁺. |
| 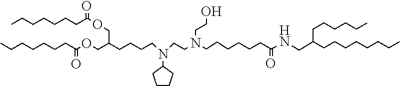 [SMILES: CCCCCCCCC(CCCCCC)CNC(=O)CCCCCCN(CCO)CCN(CCCCC(COC(=O)CCCCCCC)COC(=O)CCCCCCC)C1CCCC1] | ¹H NMR (400 MHz, CDCl₃) δ: 0.83-0.90 (m, 12H), 1.26-1.78(m, 70H), 1.93-2.01(m, 2H), 2.15-2.20 (m, 2H), 2.28-2.35(m, 4H), 2.46-2.77 (m, 9H), 3.16-3.23(m, 2H), 3.50- 3.68(m, 2H), 3.99-4.12(m, 4H). LCMS: Rt: 1.215 min; MS m/z (ESI): 906.7 [M + H]⁺. |
| Compound 230 | |
| 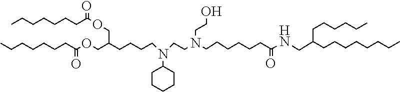 [SMILES: CCCCCCCCC(CCCCCC)CNC(=O)CCCCCCN(CCO)CCN(CCCCC(COC(=O)CCCCCCC)COC(=O)CCCCCCC)C1CCCCC1] Compound 231 | ¹H NMR (400 MHz, CDCl₃) δ: 0.86-0.90 (m, 12H), 1.26-1.47 (m, 60H), 1.53-1.66 (m, 8H), 1.75-1.87 (m, 3H), 1.93-2.01 (m, 2H), 2.15-2.19 (m, 2H), 2.28-2.40 (m, 4H), 2.41-2.80 (m, 8H), 3.03-3.30 (m, 4H), 3.50- 3.70 (m, 2H), 4.01-4.10 (m, 4H). LCMS: Rt: 1.225 min; MS m/z (ESI): 920.7 [M + H]⁺. |
| 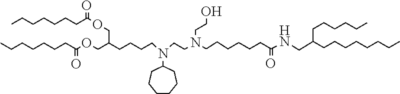 [SMILES: CCCCCCCCC(CCCCCC)CNC(=O)CCCCCCN(CCO)CCN(CCCCC(COC(=O)CCCCCCC)COC(=O)CCCCCCC)C1CCCCCC1] | ¹H NMR (400 MHz, CDCl₃) δ: 0.79-0.83 (m, 12H), 1.04-1.47 (m, 64H), 1.51-1.78 (m, 10H), 1.79-1.97 (m, 2H), 2.06-2.12 (m, 2H), 2.21-2.24 (m, 4H), 2.33-2.68 (m, 9H), 3.06-3.12 (m, 2H), 3.40-3.58 (m, 2H), 3.93- 4.06 (m, 4H). LCMS: Rt: 1.165 min; MS m/z (ESI): 934.7 [M + H]⁺. |
| Compound 232 | |
| 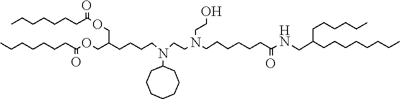 [SMILES: CCCCCCCCC(CCCCCC)CNC(=O)CCCCCCN(CCO)CCN(CCCCC(COC(=O)CCCCCCC)COC(=O)CCCCCCC)C1CCCCCCC1] | ¹H NMR (400 MHz, CDCl₃) δ: 0.83-0.90 (m, 12H), 1.26-1.47 (m, 64H), 1.57-1.75 (m, 13H), 1.86-2.05 (m, 2H), 2.15-2.28 (m, 2H), 2.30-2.40 (m, 4H), 2.50-2.82 (m, 8H), 3.16-3.22 (m, 2H), 3.43-3.60 (m, 2H), 4.00- 4.12 (m, 4H). LCMS: Rt: 1.225 min; MS m/z (ESI): 948.8 [M + H]⁺. |
| Compound 233 | |
| 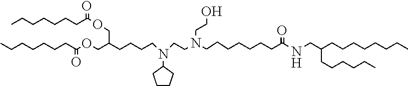 [SMILES: CCCCCCCCC(CCCCCC)CNC(=O)CCCCCCCN(CCO)CCN(CCCCC(COC(=O)CCCCCCC)COC(=O)CCCCCCC)C1CCCC1] Compound 234 | ¹H NMR (400 MHz, CDCl₃) δ: 0.86-0.90 (m, 12H), 1.26-1.47 (m, 64H), 1.61-1.63 (m, 4H), 1.73-1.78 (m, 2H), 1.94-2.01 (m, 2H), 2.11-2.19 (m, 2H), 2.28-2.32 (m, 4H), 2.43-2.63 (m, 9H), 3.01-3.07 (m, 1H), 3.14- 3.21 (m, 2H), 3.50-3.57 (m, 2H), 3.98-4.12 (m, 4H), 5.45-5.53 (m, 1H). LCMS: Rt: 1.140 min; MS m/z (ESI): 920.8 [M + H]⁺. |
| 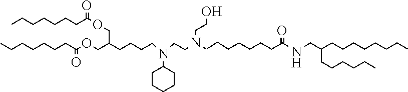 [SMILES: CCCCCCCCC(CCCCCC)CNC(=O)CCCCCCCN(CCO)CCN(CCCCC(COC(=O)CCCCCCC)COC(=O)CCCCCCC)C1CCCCC1] Compound 235 | ¹H NMR (400 MHz, CDCl₃) δ: 0.86-0.90 (m, 12H), 1.26-1.50 (m, 65H), 1.63-1.67 (m, 4H), 1.77-1.79 (m, 4H), 1.95-2.03 (m, 1H), 2.12-2.20 (m, 2H), 2.28-2.33 (m, 4H), 238-2.53 (m, 8H), 2.57-2.59 (m, 2H), 3.13- 3.21 (m, 2H), 3.51-3.54 (m, 2H), 4.00-4.11 (m, 4H), 5.45-5.52 (m, 1H). LCMS: Rt: 1.190 min; MS m/z (ESI): 934.8 [M + H]⁺. |
| 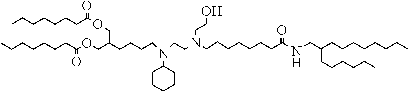 | ¹H NMR (400 MHz, CDCl₃) δ: 0.86-0.89 (m, 12H), 1.26-1.40 (m, 50H), 1.41-1.54 (m, 26H), 1.55-1.69 (m, 6H), 1.88-2.31 (m, 8H), 2.31-2.33 (m, 4H), 3.17-4.18 (m, 6H), 5.37-5.58 (m, 1H). LCMS: Rt: 1.250 min; MS m/z (ESI): 948.8 [M + H]⁺. |
| Compound 236 | |
| 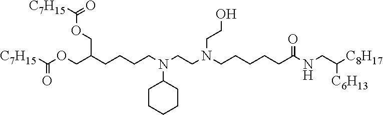 | ¹H NMR (400 MHz, CDCl₃) δ: 0.86-0.90 (m, 12H), 1.06-1.81 (m, 66H), 2.16-2.58 (m, 17H), 3.16-3.19 (m, 2H), 3.52-3.67 (m, 2H), 4.05-4.08 (m, 4H). LCMS: Rt:1.220 min; MS m/z (ESI): 878.8 [M + H]⁺. |
| Compound 237 | |
| 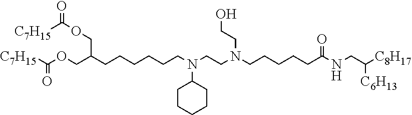 Compound 251 | ¹H NMR (400 MHz, CDCl₃) δ: 0.86-0.90 (m, 12H), 1.09-1.33 (m, 58H), 1.42-1.69 (m, 12H), 1.72-1.90 (m, 3H), 1.96-2.02 (m, 1H), 2.16-2.20 (m, 2H), 2.28-2.32 (m, 4H), 2.42-2.70 (m, 10H), 3.16-3.19 (m, 2H), 3.51-3.59 (m, 2H), 4.00-4.10 (m, 4H), 5.45- 5.57 (m, 1H). LCMS: Rt: 1.240 min; MS m/z (ESI): 934.8 [M + H]⁺. |
| 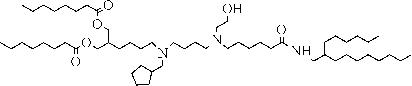 [SMILES: CCCCCCCCC(CCCCCC)CNC(=O)CCCCCN(CCO)CCCCN(CCCCC(COC(=O)CCCCCCC)COC(=O)CCCCCCC)CC1CCCC1] Compound 255 | ¹H NMR (400 MHz, CDCl₃) δ: 0.86-0.90 (m, 12H), 1.10-1.36 (m, 52H), 1.41-1.69 (m, 19H), 1.93-2.04 (m, 2H), 2.15-2.19 (m, 2H), 2.28-2.32 (m, 6H), 2.40 -2.48 (m, 8H), 2.57-2.64 (m, 2H), 3.13-3.21 (m, 2H), 3.52- 3.59 (m, 2H), 4.03-4.12 (m, 4H). LCMS: Rt: 0.985 min; MS m/z (ESI): 934.7 [M + H]⁺. |
| 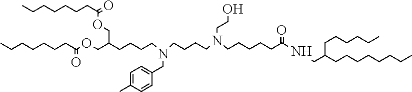 [SMILES: CCCCCCCCC(CCCCCC)CNC(=O)CCCCCN(CCO)CCCCN(CCCCC(COC(=O)CCCCCCC)COC(=O)CCCCCCC)Cc1ccc(C)cc1] | ¹H NMR (400 MHz, CDCl₃) δ: 0.86-0.89 (m, 12H), 1.26-1.34 (m, 45H), 1.59-1.69 (m, 16H), 2.31-2.58 (m, 12H), 3.17-4.01 (m, 8H), 4.03-4.05 (m, 4H), 4.05-4.08 (m, 8H), 7.13-7.17 (m, 4H). LCMS: Rt: 0.985 min; MS m/z (ESI): 956.7 [M + H]⁺. |
| Compound 256 | |

### 6.67 Example 67: Preparation of Compound 200

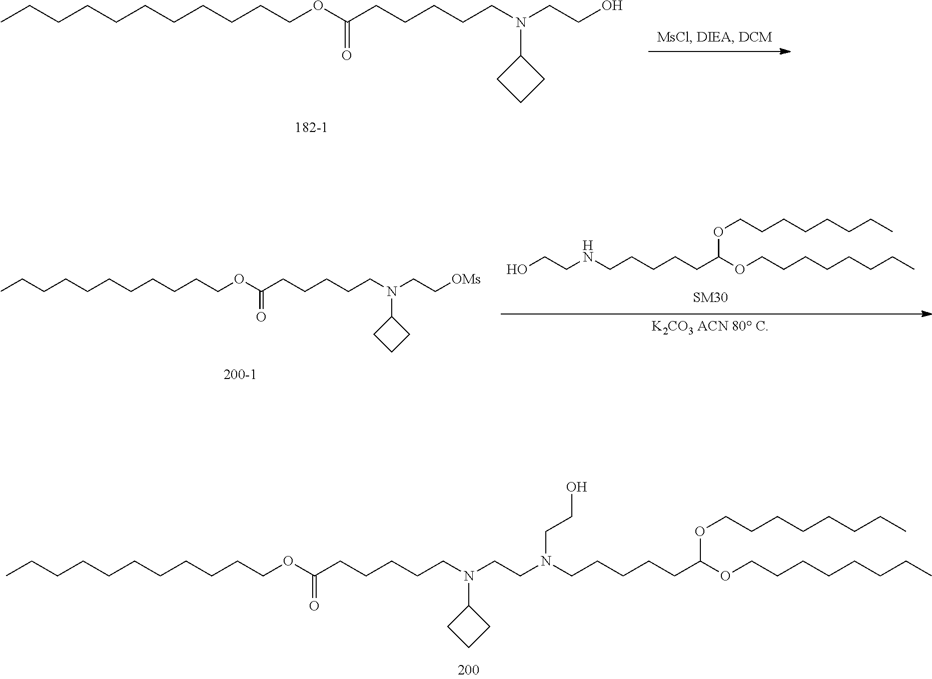 [SMILES: CCCCCCCCCCCOC(=O)CCCCCN(CCO)C1CCC1 ; "MsCl, DIEA, DCM" ; CCCCCCCCOC(CCCCCNCCO)OCCCCCCCC ; CCCCCCCCCCCOC(=O)CCCCCN(CCOS(C)(=O)=O)C1CCC1 ; "K2CO3 ACN 80° C." ; CCCCCCCCCCCOC(=O)CCCCCN(CCN(CCO)CCCCCC(OCCCCCCCC)OCCCCCCCC)C1CCC1]

### Step 1: Preparation of Compound 200-1

To a solution of compound 182-1 (650 mg, 1.7 mmol, 1.0 eq.) and DIPEA (880 mg, 6.8 mmol, 4.0 eq.) in DCM (10 mL) at 0° C. was added MsCl (390 mg, 3.4 mmol, 3.0 eq.). The mixture was stirred still for 2 hours. The mixture was poured into water and extracted with DCM. The combined organic layers were washed with brine, dried over Na₂SO₄ and concentrated to give compound 200-1 (600 mg, 76.44% yield) as yellow oil.

### Step 2: Preparation of Compound 200

A solution of compound 200-1 (600 mg, 1.3 mmol, 1.0 eq.), compound SM30 (630 mg, 1.56 mmol, 1.2 eq.), K₂CO₃ (540 mg, 3.9 mmol, 3.0 eq.), Cs₂CO₃ (130 mg, 0.39 mmol, 0.3 eq.) and NaI (60 mg, 0.39 mmol, 0.3 eq.) in ACN (10 mL) was stirred at 80° C. for overnight. TLC showed the reaction was completed. The mixture was concentrated and purified by pre-HPLC to give compound 200 (50 mg, 5.01% yield) as yellow oil.

¹H NMR (400 MHz, CDCl₃) δ: 0.86-0.89 (m, 9H), 1.26 (s, 35H), 1.41-1.46 (m, 6H), 1.54-1.66 (m, 14H), 1.85-1.99 (m, 6H), 2.27-2.31 (m, 2H), 2.39-2.59 (m, 12H), 3.04-3.08 (m, 1H), 3.36-3.42 (m, 2H), 3.52-3.58 (m, 4H), 4.03-4.07 (m, 2H), 4.43-4.46 (m, 1H). LCMS: Rt: 1.730 min; MS m/z (ESI): 767.6 [M+H]⁺.

### 6.68 Example 68: Preparation of Compound 201

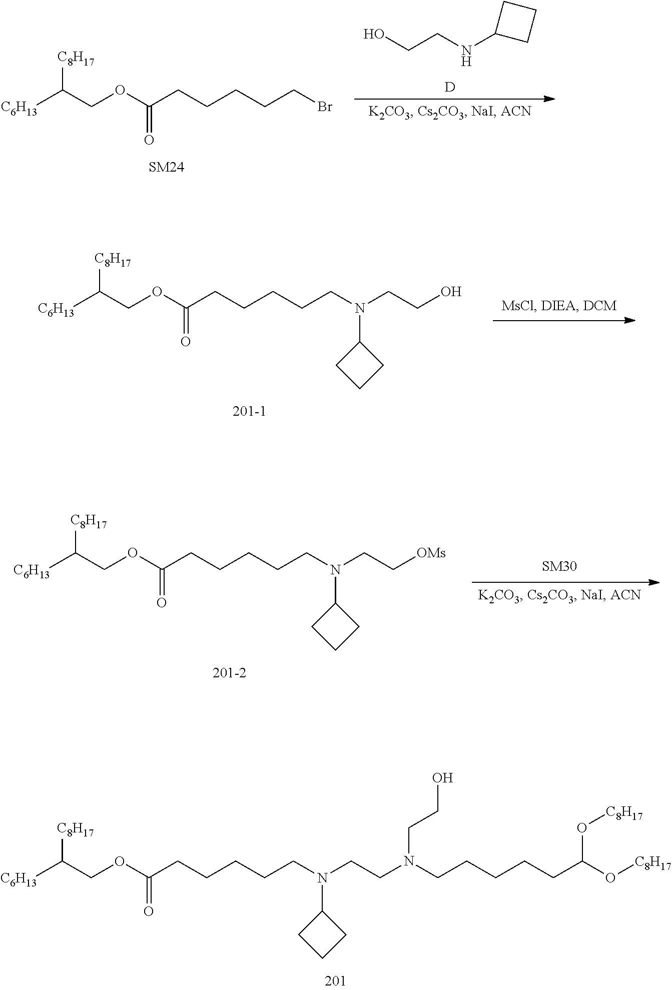

### Step 1: Preparation of Compound 201-1

To a solution of compound SM24 (1.0 g, 2.38 mmol, 1.0 eq.) and compound D (550 mg, 4.77 mmol, 2.0 eq.) in ACN (50 mL) were added K₂CO₃ (1.0 g, 7.15 mmol, 3.0 eq.), Cs₂CO₃ (230 mg, 0.71 mmol, 0.3 eq.) and NaI (110 mg, 0.71 mmol, 0.3 eq.). The mixture was stirred at 80° C. for 10 h. LCMS showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 201-1 (600 mg, 55% yield) as yellow oil. LCMS: Rt: 0.940 min; MS m/z (ESI): 454.4 [M+H]⁺.

### Step 2: Preparation of Compound 201-2

To a solution of compound 201-1 (300 mg, 0.66 mmol, 1.0 eq.) and DIPEA (260 mg, 1.99 mmol, 3.0 eq.) in DCM (20 mL) was added MsCl (115 mg, 0.99 mmol, 1.5 eq.). The mixture was stirred at room temperature for 2 hours. The mixture was poured into water and extracted with DCM. The combined organic layers were washed with brine, dried over Na₂SO₄ and concentrated to give compound 201-2 (280 mg, 80% yield) as yellow oil.

### Step 3: Preparation of Compound 201

To a solution of compound 201-2 (250 mg, 0.47 mmol, 1.0 eq.) and compound SM30 (190 mg, 0.47 mmol, 1.0 eq.) in ACN (10 mL) were added K₂CO₃ (195 mg, 1.41 mmol, 3.0 eq.), Cs₂CO₃ (46 mg, 0.14 mmol, 0.3 eq.) and NaI (21 mg, 0.14 mmol, 0.3 eq.). The mixture was stirred at 80° C. 10 h. LCMS showed the reaction was completed. The mixture was poured into water and extracted with EA. The mixture was concentrated and purified by prep-HPLC to give compound 201 (35 mg, 9% yield) as yellow oil.

¹H NMR (400 MHz, CDCl₃) δ: 0.80-0.83 (m, 12H), 1.20-1.56 (m, 66H), 1.82-1.95 (m, 4H), 2.23-2.53 (m, 12H), 2.92-3.09 (m, 1H), 3.31-3.40 (m, 2H), 3.47-3.51 (m, 4H), 3.89-3.90 (m, 2H), 4.35-4.39 (m, 1H). LCMS: Rt: 2.170 min; MS m/z (ESI): 837.7 [M+H]⁺.

### 6.69 Example 69: Preparation of Compound 202

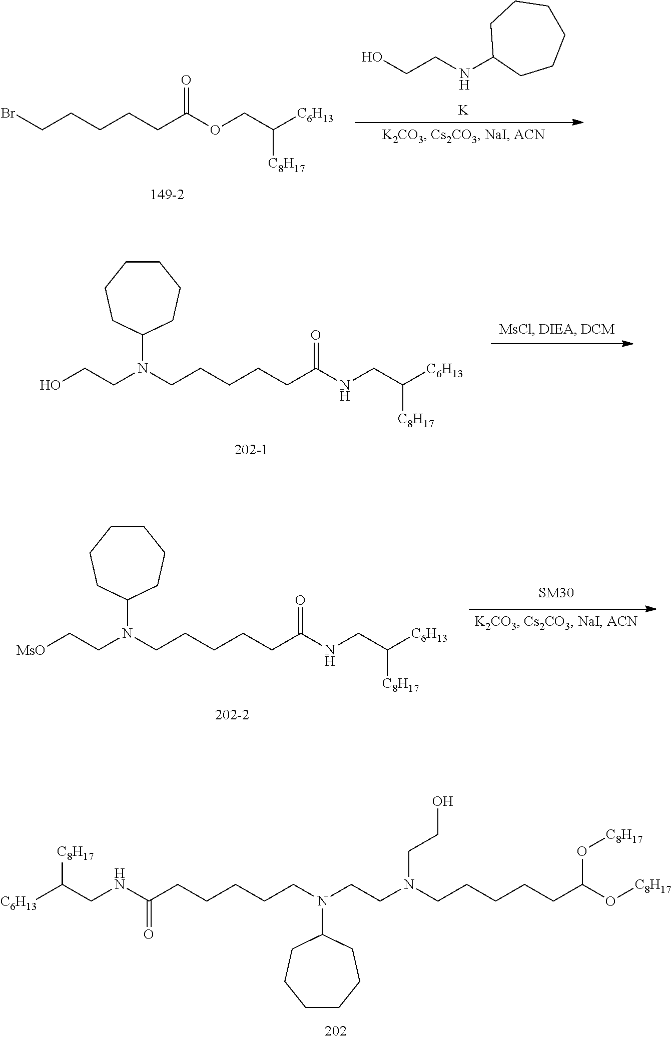

### Step 1: Preparation of Compound 202-1

To a solution of compound 149-2 (0.6 g, 1.43 mmol, 1.0 eq.) and compound K (450 mg, 2.87 mmol, 2.0 eq.) in ACN (30 mL) were added K₂CO₃ (0.6 g, 4.30 mmol, 3.0 eq.), Cs₂CO₃ (140 mg, 0.43 mmol, 0.3 eq.) and NaI (65 mg, 0.43 mmol, 0.3 eq.). The mixture was stirred at 80° C. for 10 h. LCMS showed the reaction was completed. The mixture was poured into water and extracted with EA. The combined organic layers were washed with brine, dried over Na₂SO₄ and concentrated. The residue was purified by column chromatography on silica gel (DCM/MeOH=10/1) to give compound 202-1 (350 mg, 48% yield) as yellow oil. LCMS: Rt: 0.840 min; MS m/z (ESI): 495.5 [M+H]⁺.

### Step 2: Preparation of Compound 202-2

To a solution of compound 202-1 (350 mg, 0.71 mmol, 1.0 eq.) and DIPEA (280 mg, 2.12 mmol, 3.0 eq.) in DCM (20 mL) was added MsCl (120 mg, 1.06 mmol, 1.5 eq.). The mixture was stirred at room temperature for 2 hours. The mixture was poured into water and extracted with DCM. The combined organic layers were washed with brine, dried over Na₂SO₄ and concentrated to give compound 202-2 (280 mg, 69% yield) as yellow oil.

### Step 3: Preparation of Compound 202

To a solution of compound 202-2 (250 mg, 0.44 mmol, 1.0 eq.) and compound SM30 (176 mg, 0.44 mmol, 1.0 eq.) in ACN (10 mL) were added K₂CO₃ (181 mg, 1.31 mmol, 3.0 eq.), Cs₂CO₃ (43 mg, 0.13 mmol, 0.3 eq.) and NaI (20 mg, 0.13 mmol, 0.3 eq.). The mixture was stirred at 80° C. 10 h. LCMS showed the reaction was completed. The mixture was poured into water and extracted with EA. The mixture was concentrated and purified by prep HPLC to give the title compound (25 mg, 6% yield) as yellow oil.

¹H NMR (400 MHz, CDCl₃) δ: 0.86-0.90 (m, 12H), 1.26-1.67 (m, 78H), 2.16-2.20 (m, 2H), 2.47-2.59 (m, 10H), 3.16-3.19 (m, 2H), 3.39-3.41 (m, 2H), 3.53-3.57 (m, 4H), 4.44-4.46 (m, 1H). LCMS: Rt: 1.770 min; MS m/z (ESI): 878.8 [M+H]⁺.

### 6.70 Example 70: Preparation of Compound 216

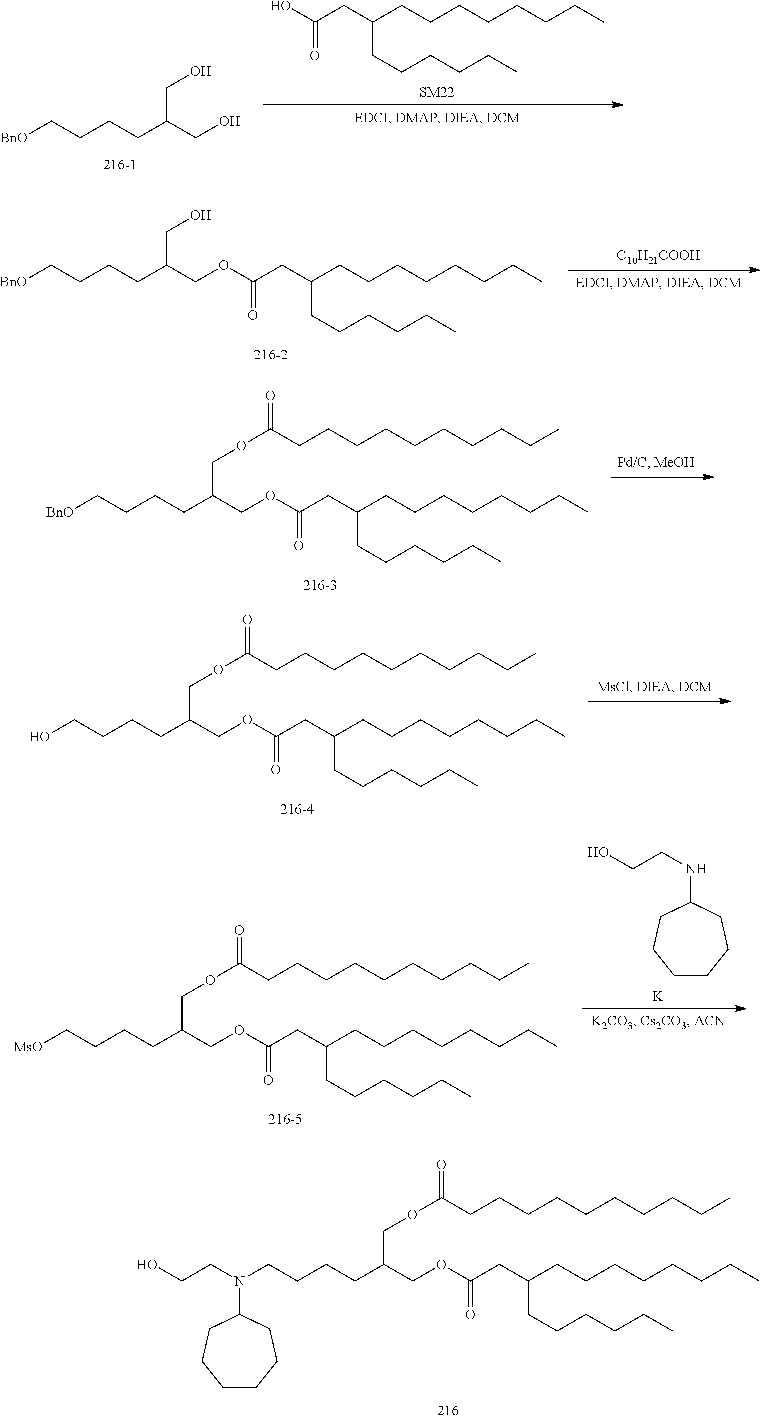

### Step 1: Preparation of Compound 216-2

To a solution of compound 216-1 (3 g, 12.6 mmol, 1.0 eq.) and compound SM22 (3 g, 11.3 mmol, 0.9 eq.) in DCM (60 mL) were added EDCI (3.6 g, 18.9 mmol, 1.5 eq.), DMAP (0.46 g, 3.78 mmol, 0.3 eq.), DIEA (4.9 g, 37.8 mmol, 3.0 eq.). The mixture was stirred at RT for 16 hours. TLC showed the reaction was complete. The reaction mixture was concentrated and purified by column chromatography on silica gel to give compound 216-2 (2.5 g, 40.43% yield) as yellow oil.

### Step 2: Preparation of Compound 216-3

To a solution of compound 216-2 (2.5 g, 5.09 mmol, 1.0 eq.) and CIOH₂₁COOH (1.1 g, 6.11 mmol, 1.2 eq.) in DCM (40 mL) were added EDCI (1.5 g, 7.64 mmol, 1.5 eq.), DMAP (0.2 g, 1.53 mmol, 0.3 eq.), DIEA (2 g, 15.3 mmol, 3.0 eq.). The mixture was stirred at 55° C. for 16 hours. TLC showed the reaction was complete. The reaction mixture was concentrated and purified by column chromatography on silica gel to give compound 216-3 (2.5 g, 74.48% yield) as colorless oil.

### Step 3: Preparation of Compound 216-4

To a solution of compound 216-3 (2.5 g, 3.79 mmol, 1.0 eq.) in EA (50 mL) was added Pd/C (300 mg). The mixture was stirred at 50° C. under H₂ for 16 hours. The mixture was filtered through a pad of Celite and washed with EA. The filtration was concentrated and purified by column chromatography on silica gel (PE/EA=3/1) to give compound 216-4 (2 g, 92.81% yield) as yellow oil.

### Step 4: Preparation of Compound 216-5

To a solution of compound 216-4 (300 mg, 0.53 mmol, 1.0 eq.) and DIPEA (210 mg, 1.59 mmol, 3.0 eq.) in DCM (10 mL) at 0° C. was added MsCl (91 mg, 0.79 mmol, 1.5 eq.). The mixture was stirred still for 2 hours. The mixture was poured into water and extracted with DCM. The combined organic layers were washed with brine, dried over Na₂SO₄ and concentrated to give compound 216-5 (350 mg, crude) as yellow oil.

### Step 5: Preparation of Compound 216

To a solution of compound 216-5 (350 mg, 0.53 mmol, 1.0 eq.) and compound K (250 mg, 1.6 mmol, 3.0 eq.) in ACN (10 mL) were added K₂CO₃ (220 mg, 1.6 mmol, 3.0 eq.), Cs₂CO₃ (50 mg, 0.16 mmol, 0.3 eq.) and NaI (20 mg, 0.16 mmol, 0.3 eq.). The mixture was stirred at 80° C. for 16 hours. LCMS showed the reaction was complete. The reaction mixture was concentrated and purified by prep HPLC to provide compound 216 (40 mg, 10.66%) as yellow oil.

¹H NMR (400 MHz, CDCl₃) δ: 0.86-0.90 (m, 9H), 1.26-1.44 (m, 48H), 1.53-1.83 (m, 12H), 1.95-1.99 (m, 1H), 2.23-2.32 (m, 4H), 2.39-2.43 (m, 2H), 2.56-2.63 (m, 3H), 3.46-3.48 (m, 2H), 4.00-4.10 (m, 4H). LCMS: Rt: 0.093 min; MS m/z (ESI): 708.5 [M+H]⁺.

The following compounds were prepared in analogous fashion as Compound 216, using corresponding starting material.

| Compound | Characterization |
|---|---|
| 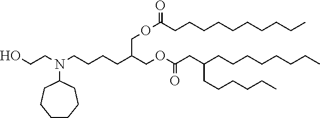 | ¹H NMR (400 MHz, CDCl₃) δ: 0.86-0.90 (m, 9H), 1.26 (s, 39H), 1.35-1.48 (m, 11H), 1.59-1.83 (m, 12H), 1.95-2.01 (m, 1H), 2.23-2.32 (m, 4H), 2.37-2.41 (m, 2H), 2.54- 2.56 (m, 2H), 2.75 (s, 1H), 3.46-3.49 (m, 2H), 4.00-4.10 (m, 4H). LCMS: Rt: 0.080 min; MS m/z (ESI): 722.5 [M + H]⁺. |
| Compound 217 | |
| 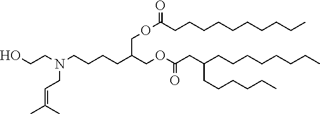 [SMILES: CCCCCCCCCCC(=O)OCC(CCCCN(CC=C(C)C)CCO)COC(=O)CC(CCCCCC)CCCCCCCC] | ¹H NMR (400 MHz, CDCl₃) δ: 0.86-0.90 (m, 9H), 1.26-1.38 (m, 43H), 1.51-1.63 (m, 7H), 1.77-1.86 (m, 4H), 1.95-2.00 (m, 1H), 2.23-2.32 (m, 4H), 2.52-2.77 (m, 4H), 3.18- 3.32 (m, 2H), 3.60-3.71 (m, 2H), 4.00-4.10 (m, 4H), 5.22-5.30 (m, 1H). LCMS: Rt: 1.370 min; MS m/z (ESI): 680.8 [M + H]⁺. |
| Compound 240 | |

### 6.71 Example 71: Preparation of Compound 218

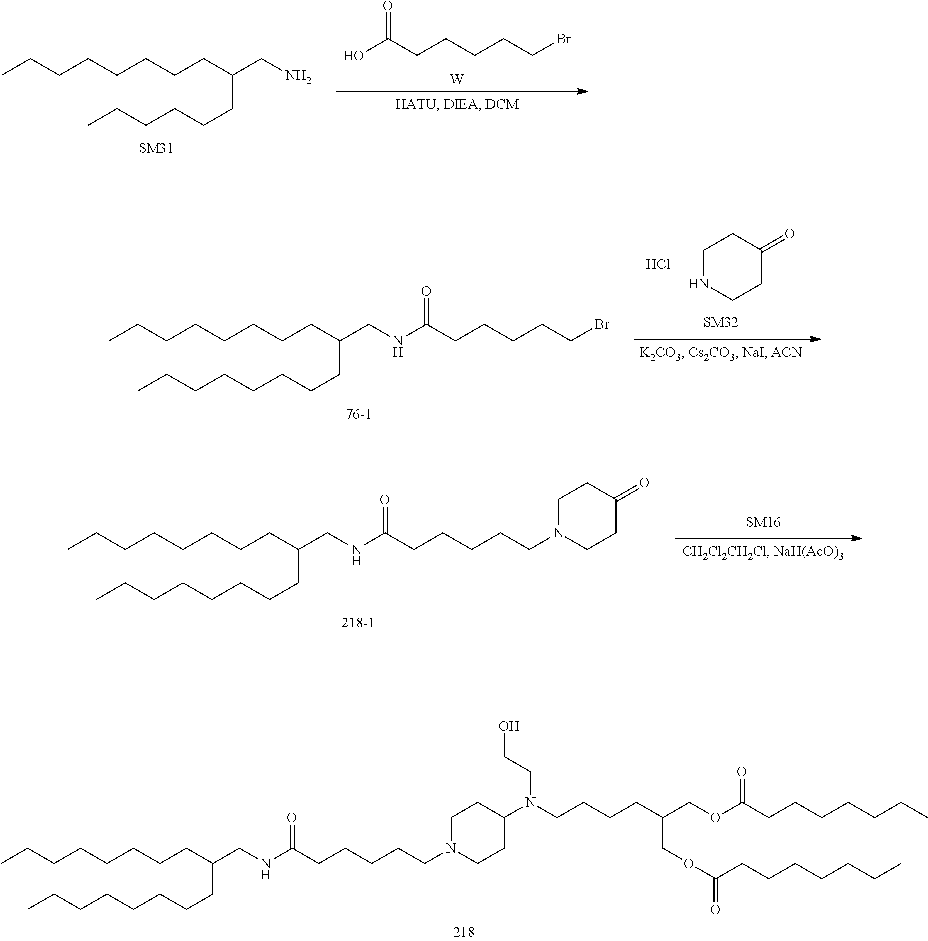

### Step 1: Preparation of Compound 76-1

To a solution of compound SM31 (2 g, 7.42 mmol, 1.0 eq.) and compound W (1.7 g, 8.91 mmol, 1.2 eq.) in DCM (40 mL) was added DIEA (1.9 g, 14.8 mmol, 2.0 eq.) and HATU (3.4 g, 8.91 mmol, 1.2 eq.). The mixture was stirred at RT for 2 hours. TLC showed the reaction was complete. The mixture was poured into water and extracted with DCM. The combined organic layers were washed with brine, dried over Na₂SO₄, concentrated and purified by column chromatography on silica gel to give compound 76-1 (3.0 g, 90.52% yield) as yellow oil.

### Step 2: Preparation of Compound 218-1

To a solution of compound 76-1 (800 mg, 1.79 mmol, 1.0 eq.) and compound SM32 (270 mg, 1.97 mmol, 1.1 eq.) in ACN (20 mL) were added K₂CO₃ (740 mg, 5.37 mmol, 3.0 eq.), Cs₂CO₃ (180 mg, 0.54 mmol, 0.3 eq.) and NaI (27 mg, 0.18 mmol, 0.1 eq.). The mixture was stirred at 80° C. for 16 hours. LCMS showed the reaction was complete. The reaction mixture was concentrated and FCC to provide compound 218-1 (450 mg, 54.06%) as yellow oil.

### Step 3: Preparation of Compound 218

To a solution of compound 218-1 (220 mg, 0.47 mmol, 1.0 eq.) and compound SM16 (210 mg, 0.47 mmol, 1.0 eq.) in DCE (10 mL) were added AcOH (1 drop). The mixture was stirred at rt for 16 hours. Then NaBH(OAc)₃ (300 mg, 1.42 mmol, 3.0 eq.) was added. The mixture was stirred at rt for 24 hours. LCMS showed the reaction was completed. The mixture was poured into water and extracted with EA. The combined organic layers were washed with brine, dried over Na₂SO₄ and concentrated. The residue was purified by prep-HPLC to give compound 218 (50 mg, 11.84% yield) as yellow oil.

¹H NMR (400 MHz, CDCl₃) δ: 0.86-0.90 (m, 12H), 1.26-1.34 (m, 50H), 1.41-1.52 (m 5H), 1.59-1.67 (m, 10H), 1.85-2.00 (m, 4H), 2.15-2.18 (m, 2H), 2.28-2.32 (m, 6H), 2.45-2.49 (m, 3H), 2.61-2.64 (m, 2H), 2.98 (d, J=11.2 Hz, 2H), 3.16-3.19 (m, 2H), 3.46-3.49 (m, 2H), 4.00-4.09 (m, 4H), 5.33-5.36 (m, 1H). LCMS: Rt: 0.093 min; MS m/z (ESI): 892.7 [M+H]⁺.

### 6.72 Example 72: Preparation of Compound 223

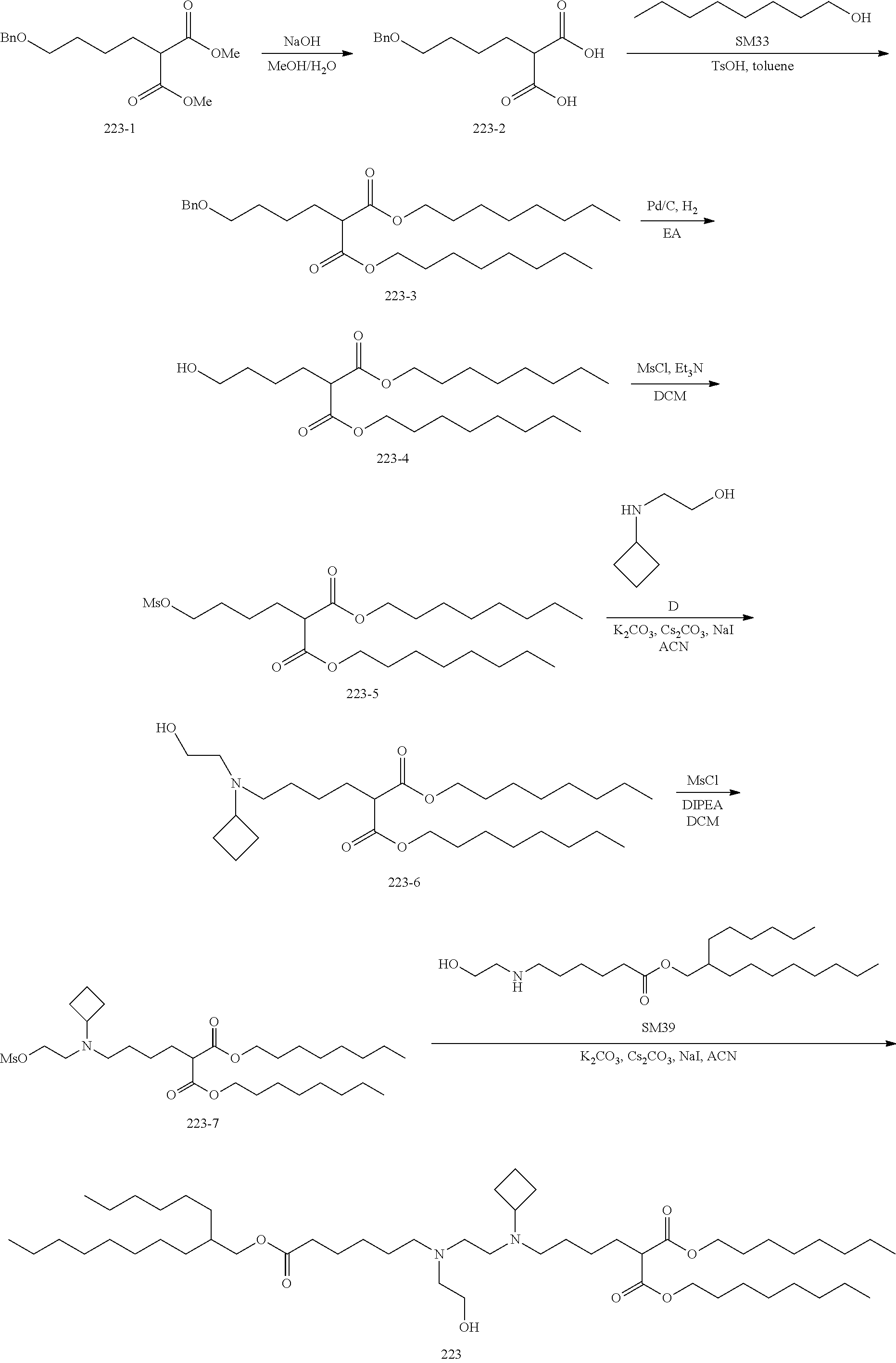

### Step 1: Preparation of Compound 223-2

To a solution of compound 223-1 (7.0 g, 23.0 mmol, 1.0 eq.) in MeOH/H₂O (50 mL/50 mL) was added NaOH (7.6 g, 184.0 mmol, 8.0 eq.). The mixture was stirred at 25° C. for 10 hours. TLC showed the reaction was completed. The reaction mixture was concentrated to remove the organic layer. The aqueous layer was adjusted pH to 5 with 2N HCl and then extracted with EA. The combined organic layers were washed with brine, dried over Na₂SO₄ and concentrated to give compound 223-2 (4.6 g, 72% yield) as colorless oil.

### Step 2: Preparation of Compound 223-3

To a solution of compound 223-2 (4.6 g, 17.0 mmol, 1.0 eq.) and compound SM33 (5.4 g, 42.0 mmol, 2.5 eq.) in toluene (60 mL) was added TsOH (0.32 g, 1.7 mmol, 0.1 eq.). The mixture was stirred under reflux through Dean-S-tark trap for 3 hours. TLC showed the reaction was completed. The mixture was poured into water and extracted with EA. The combined organic layers were washed with brine, dried over Na₂SO₄ and concentrated. The residue was purified by column chromatography on silica gel to give compound 223-3 (6.3 g, 74% yield) as colorless oil.

### Step 3: Preparation of Compound 223-4

To a solution of compound 223-3 (2.4 g, 4.8 mmol, 1.0 eq.) in EA (25 mL) was added Pd/C (0.2 g) and two drops of con. HCl. The mixture was stirred at 25° C. under H₂ for 5 hours. TLC showed the reaction was completed. The reaction mixture was filtered through a pad of Celite and washed with EA. The filtrate was concentrated to give compound 223-4 (2 g, 92% yield) as colorless oil.

### Step 4: Preparation of Compound 223-5

To a solution of compound 223-4 (2.0 g, 5.0 mmol, 1.0 eq.) and TEA (1.0 g, 10.0 mmol, 2.0 eq.) in DCM (50 mL) was added MsCl (687 mg, 6.0 mmol, 1.2 eq.). The mixture was stirred at rt for 2 hours. The mixture was poured into water and extracted with DCM. The combined organic layers were washed with brine, dried over Na₂SO₄ and concentrated to give compound 223-5 (12.4 g, 100% yield) as yellow oil.

### Step 5: Preparation of Compound 223-6

To a solution of compound 223-5 (300 mg, 0.63 mmol, 1.0 eq.) and compound D (145 mg, 126 mmol, 2.0 eq.) in ACN (12 mL) were added K₂CO₃ (261 mg, 1.89 mmol, 3.0 eq.), Cs₂CO₃ (62 mg, 0.19 mmol, 0.3 eq.) and NaI (28 mg, 0.19 mmol, 0.3 eq.). The mixture was stirred at 80° C. for 16 hours. LCMS showed the reaction was completed. The mixture was poured into water and extracted with EA. The combined organic layers were washed with brine, dried over Na₂SO₄ and concentrated. The residue was purified by column chromatography on silica gel (DCM/MeOH=25/1) to give compound 223-6 (126 mg) as yellow oil, which was further purified by prep-HPLC to give compound 223-6 (65 mg, 21% yield) as yellow oil. ¹H NMR (400 MHz, CDCl₃) δ: 0.86-0.90 (m, 6H), 1.27-1.32 (m, 22H), 1.45-1.74 (m, 9H), 1.87-2.10 (m, 6H), 2.44-2.65 (m, 4H), 3.14-3.19 (m, 1H), 3.24-3.31 (m, 1H), 3.54-3.61 (m, 2H), 4.08-4.17 (m, 4H). LCMS: Rt: 0.860 min; MS m/z (ESI): 498.5 [M+H]⁺.

### Step 6: Preparation of Compound 223-7

To a solution of compound 223-6 (1.0 g, 2.0 mmol, 1.0 eq.) and DIPEA (517 mg, 4.0 mmol, 2.0 eq.) in DCM (20 mL) was added MsCl (275 mg, 2.4 mmol, 1.2 eq.). The mixture was stirred at 0° C. for 2 hours. The mixture was poured into water and extracted with DCM. The combined organic layers were washed with brine, dried over Na₂SO₄ and concentrated to give compound 223-7 (1.1 g, 95% yield) as yellow oil.

### Step 7: Preparation of Compound 223

To a solution of compound 223-7 (1.0 g, 1.74 mmol, 1.0 eq.) and compound SM39 (694 mg, 1.74 mmol, 1.0 eq.) in ACN (30 mL) were added K₂CO₃ (721 mg, 5.22 mmol, 3.0 eq.), Cs₂CO₃ (169 mg, 0.52 mmol, 0.3 eq.) and NaI (78 mg, 0.52 mmol, 0.3 eq.). The mixture was stirred at 80° C. for 16 hours. LCMS showed the reaction was completed. The mixture was poured into water and extracted with EA. The combined organic layers were washed with brine, dried over Na₂SO₄ and concentrated. The residue was purified by column chromatography on silica gel (DCM/MeOH=25/1) to give compound 223 (520 mg, 35% yield) as yellow oil. 100 mg product was further purified by prep-HPLC to give compound 223 (45 mg, 45% yield) as yellow oil.

¹H NMR (400 MHz, CDCl₃) δ: 0.86-0.90 (m, 12H), 1.26-1.30 (m, 49H), 1.44-1.68 (m, 12H), 1.87-2.14 (m, 5H), 2.29-3.00 (m, 15H), 3.30-3.33 (m, 1H), 3.53-3.69 (m, 2H), 3.96-3.97 (m, 2H), 4.11-4.17 (m, 4H). LCMS: Rt: 1.620 min; MS m/z (ESI): 879.7 [M+H]⁺.

### 6.73 Example 73: Preparation of Compound 238

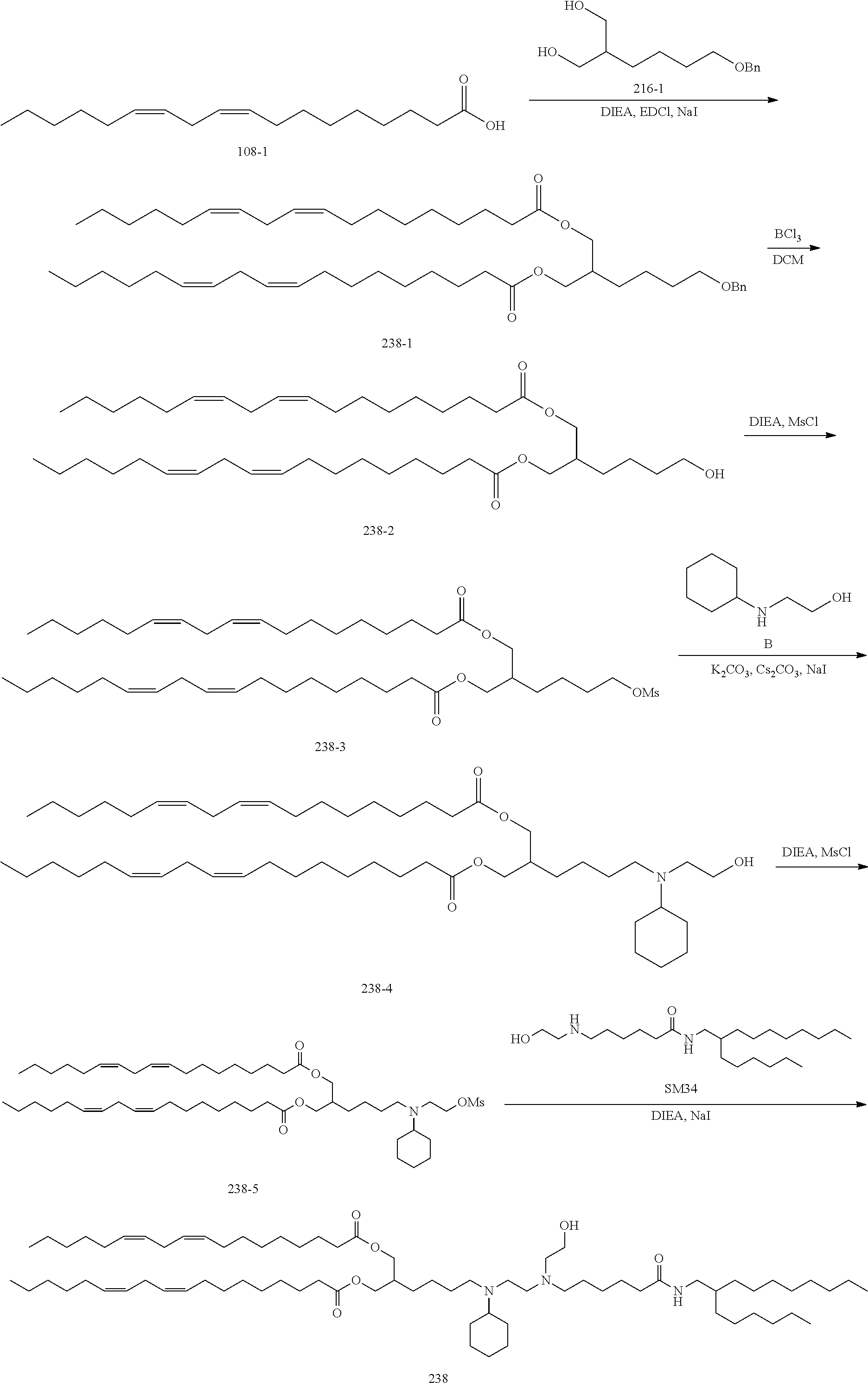

### Step 1: Preparation of Compound 238-1

To a solution of compound 108-1 (3.53 g, 12.59 mmol, 1.0 eq.) and compound 216-1 (1.0 g, 4.20 mmol, 0.3 eq.) in DCM (50 mL) were added DIEA (2.71 g, 20.98 mmol, 1.7 eq.), EDCI (2.41 g, 12.59 mmol, 1.0 eq.) and DMAP (0.52 g, 4.20 mmol, 0.3 eq.). The mixture was stirred at 45° C. for 10 hours. The reaction mixture was concentrated and purified by column chromatography on silica gel (PE/EA=10:1) to give compound 238-1 (2.0 g, 62% yield) as colorless oil.

### Step 2: Preparation of Compound 238-2

To a solution of compound 238-1 (1.0 g, 1.31 mmol, 1.0 eq.) in DCM (20 mL) was added BCl₃ (15.6 mL, 15.6 mmol, 12.0 eq.) at −78° C. The mixture was stirred at −78° C. for 1 hours. The mixture was poured into aqueous NaHCO₃ and extracted with DCM. The combined organic layers were washed with brine, dried over Na₂SO₄, concentrated and purified by column chromatography on silica gel (PE/EA=4:1) to give compound 238-2 (0.5 g, 57% yield) as colorless oil.

### Step 3: Preparation of Compound 238-3

To a solution of compound 238-2 (800 mg, 1.19 mmol, 1.0 eq.) and DIPEA (310 mg, 2.38 mmol, 2.0 eq.) in DCM (20 mL) was added MsCl (165 mg, 1.43 mmol, 1.2 eq.). The mixture was stirred at room temperature for 2 hours. The mixture was poured into water and extracted with DCM. The combined organic layers were washed with brine, dried over Na₂SO₄ and concentrated to give compound 238-3 (600 mg, 67% yield) as yellow oil, which was used in the next step without further purification.

### Step 4: Preparation of Compound 238-4

To a solution of compound 238-3 (600 mg, 0.8 mmol, 1.0 eq.) and compound B (230 mg, 1.6 mmol, 2.0 eq.) in ACN (30 mL) were added K₂CO₃ (332 mg, 2.4 mmol, 3.0 eq.), Cs₂CO₃ (79 mg, 0.24 mmol, 0.3 eq.) and NaI (36 mg, 0.24 mmol, 0.2 eq.). The mixture was stirred at 80° C. for 10 hours. LCMS showed the reaction was complete. The reaction mixture was concentrated and purified by column chromatography on silica gel (DCM/MeOH=20/1) to give compound 238-4 (500 mg, 78% yield) as yellow oil. LCMS: Rt: 1.380 min; MS m/z (ESI): 798.6 [M+H]⁺.

### Step 5: Preparation of Compound 238-5

To a solution of compound 238-4 (250 mg, 0.31 mmol, 1.0 eq.) and DIPEA (122 mg, 0.94 mmol, 3.0 eq.) in DCM (20 mL) was added MsCl (44 mg, 0.38 mmol, 1.0 eq.). The mixture was stirred at room temperature for 2 hours. The mixture was poured into water and extracted with DCM. The combined organic layers were washed with brine, dried over Na₂SO₄ and concentrated to give compound 238-5 (220 mg, 80% yield) as yellow oil, which was used in the next step without further purification.

### Step 6: Preparation of Compound 238

To a solution of compound 238-5 (200 mg, 0.23 mmol, 1.0 eq.) and compound SM34 (91 mg, 0.23 mmol, 1.0 eq.) in ACN (10 mL) were added K₂CO₃ (95 mg, 0.69 mmol, 3.0 eq.), Cs₂CO₃ (23 mg, 0.07 mmol, 0.3 eq.) and NaI (11 mg, 0.07 mmol, 0.3 eq.). The mixture was stirred at 80° C. for 10 hours. LCMS showed the reaction was complete. The reaction mixture was concentrated and purified by Prep-HPLC to give compound 238 (11 mg, 4% yield) as yellow oil.

¹H NMR (400 MHz, CDCl₃) δ: 0.79-0.84 (m, 12H), 1.19-1.74 (m, 81H), 1.95-2.72 (m, 30H), 3.09-3.13 (m, 2H), 3.13-3.50 (m, 2H), 3.92-4.09 (m, 4H), 5.27-5.30 (m, 8H). LCMS: Rt: 0.627 min; MS m/z (ESI): 1179.0 [M+H]⁺.

### 6.74 Example 74: Preparation of Compound 239

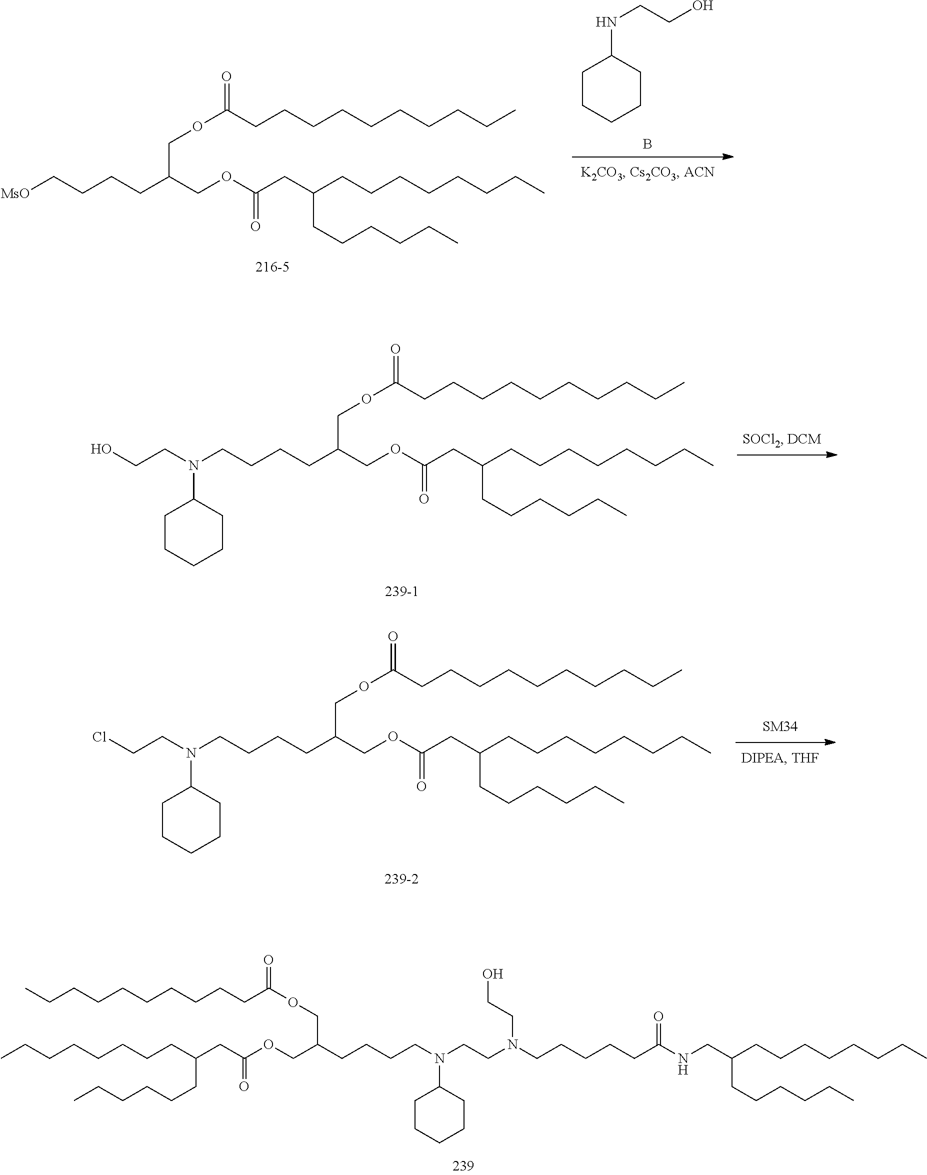

### Step 1: Preparation of Compound 239-1

To a solution of compound 216-5 (820 mg, 1.27 mmol, 1.0 eq.) and compound B (273 mg, 1.91 mmol, 1.5 eq.) in ACN (25 mL) were added K₂CO₃ (527 mg, 3.81 mmol, 3.0 eq.), Cs₂CO₃ (124 mg, 0.38 mmol, 0.3 eq.) and NaI (57 mg, 0.38 mmol, 0.3 eq.). The mixture was stirred at 80° C. for 16 hours. LCMS showed the reaction was complete. The mixture was poured into water and extracted with EA. The combined organic layers were washed with brine, dried over Na₂SO₄ and concentrated. The residue was purified by column chromatography on silica gel (DCM/MeOH=50/1) to give compound 239-1 (290 mg, 33% yield) as yellow oil. LCMS: Rt: 1.420 min; MS m/z (ESI): 694.6 [M+H]⁺.

### Step 2: Preparation of Compound 239-2

To a solution of compound 239-1 (290 mg, 0.42 mmol, 1.0 eq.) in DCM (10 mL) was added SOCl₂ (150 mg, 1.26 mmol, 3.0 eq.). The mixture was stirred at 30° C. for 16 hours. LCMS showed the reaction was completed. The mixture was concentrated to give compound 239-2 (298 mg, 100% yield) as yellow oil, which was used in the next step without further purification. LCMS: Rt: 1.700 min; MS m/z (ESI): 712.6 [M+H]⁺.

### Step 3: Preparation of Compound 239

To a solution of compound 239-2 (270 mg, 0.38 mmol, 1.0 eq.) and compound SM34 (151 mg, 0.38 mmol, 1.0 eq.) in THF (10 mL) were added DIPEA (147 mg, 1.14 mmol, 3.0 eq.) and NaI (17 mg, 0.114 mmol, 0.3 eq.). The mixture was stirred at 70° C. for 16 hours. LCMS showed the reaction was completed. The mixture was concentrated and purified by prep-HPLC to give compound 239 (80 mg, 20% yield) as yellow oil.

¹H NMR (400 MHz, CDCl₃) δ: 0.86-0.90 (m, 15H), 1.26-1.36 (m, 76H), 1.37-1.70 (m, 10H), 1.74-1.94 (m, 4H), 1.97-2.00 (m, 1H), 2.11-2.32 (m, 7H), 2.37-2.71 (m, 8H), 2.93-3.07 (m, 2H), 3.16-3.19 (m, 2H), 3.50-3.64 (m, 2H), 3.99-4.10 (m, 4H). LCMS: Rt: 0.507 min; MS m/z (ESI): 1074.9 [M+H]⁺.

### 6.75 Example 75: Preparation of Compound 241

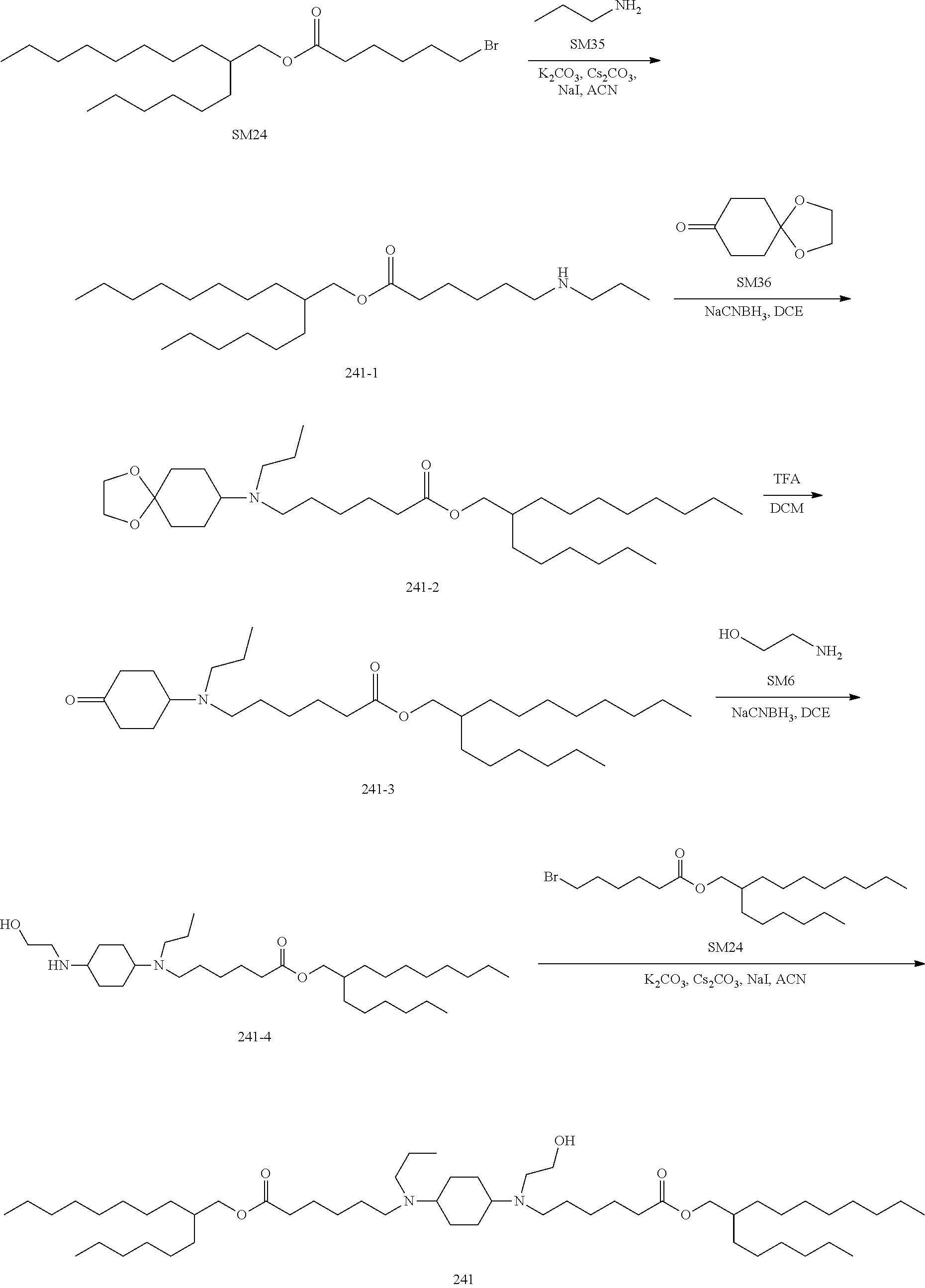

### Step 1: Preparation of Compound 241-1

To a solution of compound SM24 (2.0 g, 4.8 mmol, 1.0 eq.) and compound SM35 (709 mg, 12.0 mmol, 2.5 eq.) in ACN (50 mL) were added K₂CO₃ (2.0 g, 14.4 mmol, 3.0 eq.), Cs₂CO₃ (469 mg, 1.44 mmol, 0.3 eq.) and NaI (216 mg, 1.44 mmol, 0.3 eq.). The mixture was stirred at 80° C. for 16 hours. LCMS showed the reaction was complete. The mixture was concentrated and purified by column chromatography on silica gel (DCM/MeOH=25/1) to give compound 241-1 (830 mg, 44% yield) as yellow oil. LCMS: Rt: 0.850 min; MS m/z (ESI): 398.5 [M+H]⁺.

### Step 2: Preparation of Compound 241-2

To a solution of compound 241-1 (400 mg, 1.0 mmol, 1.0 eq.) and compound SM36 (157 mg, 1.0 mmol, 1.0 eq.) in DCE (10 mL) were added AcOH (1 drop). The mixture was stirred at rt for 4 hours. Then NaBH₃CN (94 mg, 1.5 mmol, 1.5 eq) was added. The mixture was stirred at rt for 16 hours. LCMS showed the reaction was completed. The mixture was concentrated and purified by column chromatography on silica gel (DCM/MeOH=30/1) to give compound 241-2 (282 mg, 52% yield) as yellow oil. LCMS: Rt: 0.467 min; MS m/z (ESI): 538.5 [M+H]⁺.

### Step 3: Preparation of Compound 241-3

To a solution of compound 241-2 (250 mg, 0.46 mmol, 1.0 eq.) in DCM (9 mL) was added TFA (3.0 mL). The mixture was stirred at rt for 24 hours. LCMS showed the reaction was completed. The mixture was adjusted to pH=8 with saturated NaHCO₃ solution, then extracted with DCM. The combined organic layers were washed with brine, dried over Na₂SO₄ and concentrated to give compound 241-3 (223 mg, 97% yield) as brown oil. LCMS: Rt: 0.920 min; MS m/z (ESI): 494.5 [M+H]⁺.

### Step 4: Preparation of Compound 241-4

To a solution of compound 241-3 (230 mg, 0.47 mmol, 1.0 eq.) and compound SM6 (57 mg, 0.94 mmol, 2.0 eq.) in DCE (10 mL) were added AcOH (1 drop). The mixture was stirred at rt for 4 hours. Then NaBH₃CN (44 mg, 0.71 mmol, 1.5 eq) was added. The mixture was stirred at rt for 16 hours. LCMS showed the reaction was complete. The mixture was poured into water and extracted with DCM. The combined organic layers were washed with brine, dried over Na₂SO₄ and concentrated. The residue was concentrated and purified by column chromatography on silica gel (DCM/MeOH=10/1) to give compound 241-4 (144 mg, 57% yield) as yellow oil. LCMS: Rt: 0.160 min; MS m/z (ESI): 539.5 [M+H]⁺.

### Step 5: Preparation of Compound 241

To a solution of compound 241-4 (144 mg, 0.27 mmol, 1.0 eq.) and compound SM24 (227 mg, 0.54 mmol, 2.0 eq.) in ACN (10 mL) were added K₂CO₃ (112 mg, 0.81 mmol, 3.0 eq.), Cs₂CO₃ (26 mg, 0.081 mmol, 0.3 eq.) and NaI (12 mg, 0.081 mmol, 0.3 eq.). The mixture was stirred at 80° C. for 16 hours. LCMS showed the reaction was complete. The mixture was concentrated and purified by prep-HPLC to give compound 241 (51 mg, 22% yield) as yellow oil.

¹H NMR (400 MHz, CDCl₃) δ: 0.86-0.90 (m, 15H), 1.27 (s, 56H), 1.38-1.41 (m, 6H), 1.51-1.63 (m, 7H), 1.74-1.91 (m, 4H), 2.29-2.71 (m, 14H), 3.43-3.52 (m, 2H), 3.96-3.97 (m, 4H). LCMS: Rt: 0.400 min; MS m/z (ESI): 877.8 [M+H]⁺.

### 6.76 Example 76: Preparation of Compound 244

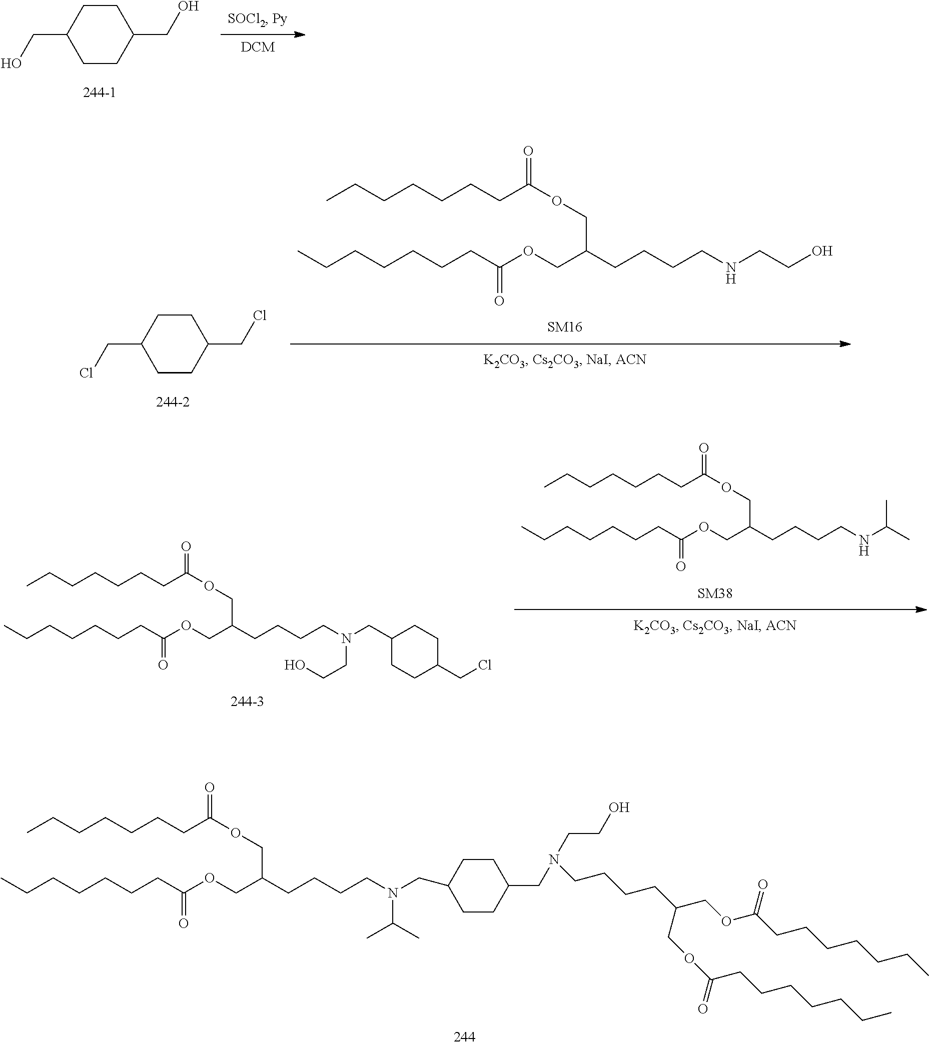

### Step 1: Preparation of Compound 244-2

A mixture of 244-1 (4.0 g, 27.7 mmol, 1.0 eq.), SOCl₂ (9.9 g, 83.2 mmol, 3.0 eq.), pyridine (6.6 g, 83.2 mmol, 3.0 eq.) in DCM (50 mL) was stirred at reflux for 4 h. TLC showed the reaction was completed. The mixture was diluted with DCM and washed with water and brine, dried and concentrated. The residue was purified by FCC to give compound 244-2 (4.5 g, 89.7% yield) as colorless oil.

### Step 2: Preparation of Compound 244-3

A mixture of 244-2 (612 mg, 3.38 mmol, 1.0 eq.), SM16 (500 mg, 1.13 mmol, 3.0 eq.), K₂CO₃ (466 mg, 3.38 mmol, 3.0 eq.), Cs₂CO₃ (111 mg, 0.34 mmol, 3.0 eq.), NaI (51 mg, 0.34 mmol, 3.0 eq.) in ACN (10 mL) was stirred at reflux for overnight. LCMS showed the product. The mixture was diluted with EA and washed with water and brine, dried and concentrated. The residue was purified by FCC to give compound 244-3 (260 mg, 39.1% yield) as colorless oil.

### Step 3: Preparation of Compound 244-4

A mixture of 244-3 (260 mg, 0.44 mmol, 1.0 eq), SM38 (234 mg, 0.53 mmol, 1.2 eq), K₂CO₃ (184 mg, 1.33 mmol, 3.0 eq), Cs₂CO₃ (42 mg, 0.13 mmol, 3.0 eq), NaI (20 mg, 0.13 mmol, 3.0 eq) in ACN (5 mL) was stirred at reflux for overnight. LCMS showed the product. The mixture was diluted with EA and washed with water and brine, dried and concentrated. The residue was purified by pre-HPLC to give compound 244 (38 mg, 8.7% yield) as yellow oil.

¹H NMR (400 MHz, CDCl₃) δ: 0.86-1.08 (m, 18H), 1.28-1.36 (m, 52H), 1.61-1.63 (m, 8H), 1.80-2.04 (m, 7H), 2.26-2.32 (m, 11H), 2.44-2.76 (m, 4H) 2.84-3.01 (m, 2H), 3.48-3.70 (m, 2H), 3.99-4.11 (m, 8H). LCMS: Rt: 1.380 min; MS m/z (ESI): 993.8 [M+H]⁺.

### 6.77 Example 77: Preparation of Compound 246

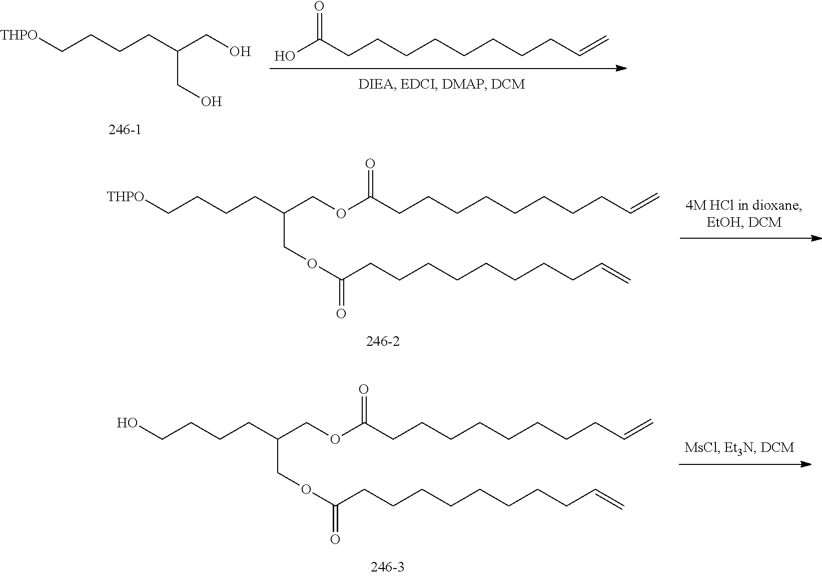

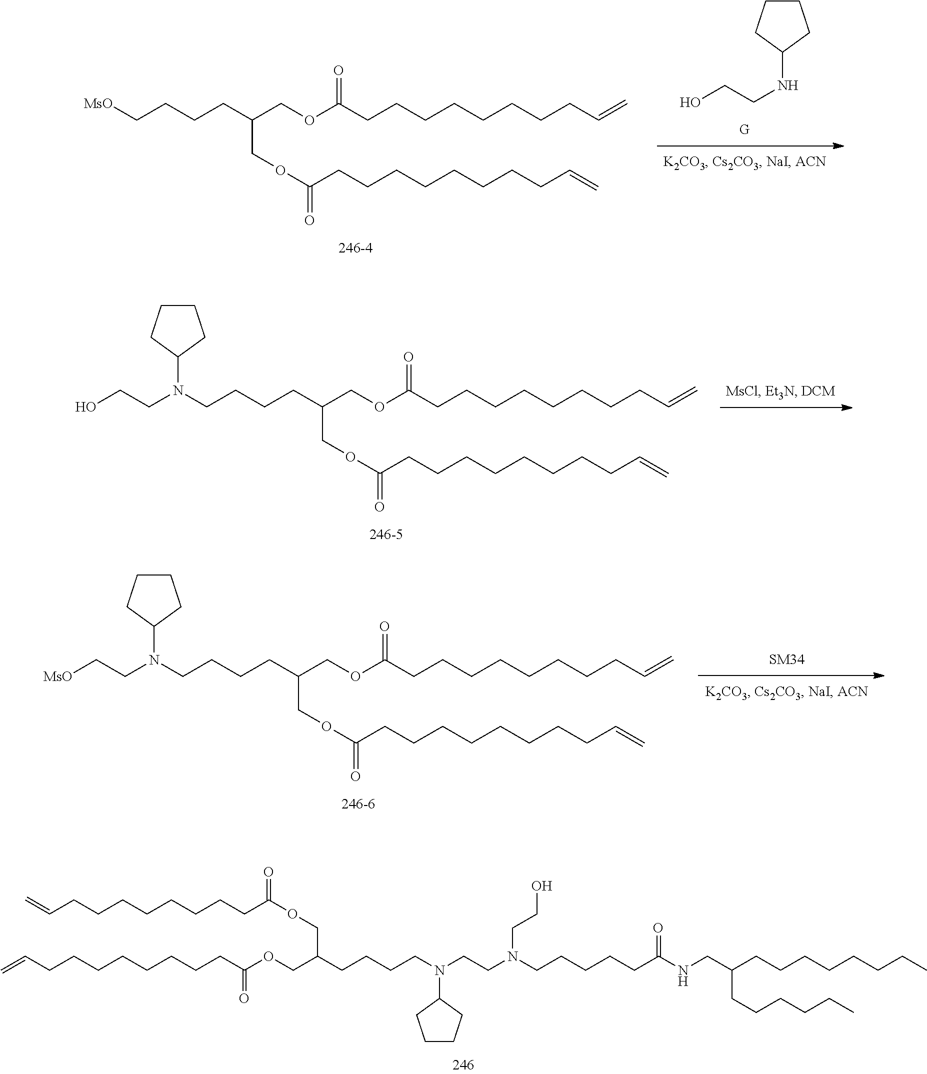

### Step 1: Preparation of Compound 246-2

To a solution of 246-1 (1.7 g, 7.3 mmol, 1.0 eq.) and undec-10-enoic acid (4.0 g, 22.0 mmol, 3.0 eq.) in DCM (40 mL) were added DIEA (4.7 g, 36.6 mmol, 5.0 eq.), EDCI (4.2 g, 22.0 mmol, 3.0 eq.) and DMAP (268 mg, 2.2 mmol, 0.3 eq.). The mixture was stirred at 40° C. for 16 hours. The reaction mixture was concentrated and purified by column chromatography on silica gel (PE/EA=10:1) to give compound 246-2 (1.7 g, 41.1% yield) as colorless oil.

### Step 2: Preparation of Compound 246-3

To a solution of 246-2 (1.7 g, 3.01 mmol, 1.0 eq.) in DCM (34 mL) was added 4 M HCl in dioxane (5 mL, 20 mmol, 6.6 eq.) at 0° C. The mixture was stirred at 0° C. for 40 min. The mixture was poured into NaHCO₃ (aq) and extracted with DCM. The combined organic layers were washed with brine, dried over Na₂SO₄, concentrated and purified by column chromatography on silica gel (EA/DCM=1/10) to give compound 246-3 (869 mg, 60.1% yield) as colorless oil.

### Step 3: Preparation of Compound 246-4

To a solution of 246-3 (869 mg, 1.8 mmol, 1.0 eq.) and Et₃N (350 mg, 2.7 mmol, 1.5 eq.) in DCM (16 mL) was added MsCl (228 mg, 2.0 mmol, 0.1 eq.). The mixture was stirred at room temperature for 1 hour. The mixture was poured into water and extracted with DCM. The combined organic layers were washed with brine, dried over Na₂SO₄ and concentrated to give compound 246-4 (1.1 g, crude) as colorless oil. It was used in the next step without further purification.

### Step 4: Preparation of Compound 246-5

To a solution of 246-4 (1.1 g, 2.0 mmol, 1.0 eq.) and compound G (756 mg, 5.9 mmol, 3.0 eq.) in ACN (30 mL) were added K₂CO₃ (815 mg, 5.9 mmol, 3.0 eq.), Cs₂CO₃ (19 mg, 0.06 mmol, 0.03 eq.) and NaI (148 mg, 1.0 mmol, 0.5 eq.). The mixture was stirred at 80° C. for 16 hours. LCMS showed the reaction was complete. The reaction mixture was concentrated and purified by column chromatography on silica gel (DCM/MeOH=30/1) to give compound 246-5 (899 mg, 77.2% yield) as colorless oil. LCMS: Rt: 1.630 min; MS m/z (ESI): 592.5 [M+H]⁺.

### Step 5: Preparation of Compound 246-6

To a solution of 246-5 (300 mg, 0.5 mmol, 1.0 eq.) and Et₃N (98 mg, 0.8 mmol, 1.6 eq.) in DCM (6 mL) was added MsCl (69 mg, 0.6 mmol, 1.2 eq.). The mixture was stirred at room temperature for 1 hours. The mixture was poured into water and extracted with DCM. The combined organic layers were washed with brine, dried over Na₂SO₄ and concentrated to give compound 246-6 (362 mg, crude) as orange oil. It was used in the next step without further purification.

### Step 6: Preparation of Compound 246

To a solution of 246-6 (342 mg, 0.5 mmol, 1.0 eq.) and SM34 (210 mg, 0.5 mmol, 1.0 eq.) in ACN (10 mL) were added K₂CO₃ (211 mg, 1.5 mmol, 3.0 eq.), Cs₂CO₃ (5 mg, 0.02 mmol, 0.04 eq.) and NaI (38 mg, 0.26 mmol, 0.52 eq.). The mixture was stirred at 80° C. for 16 hours. LCMS showed the reaction was complete. The reaction mixture was concentrated and purified by prep-HPLC to give compound 246 (11 mg, 7.22% yield) as colorless oil.

¹H NMR (400 MHz, CDCl₃) δ: 0.86-0.89 (m, 6H), 1.26-1.39 (m, 51H), 1.49-1.68 (m, 14H), 1.95-2.06 (m, 8H), 2.18-2.32 (m, 9H), 2.52-2.69 (m, 7H), 2.95-3.19 (m, 6H), 3.49-3.67 (m, 2H), 4.00-4.10 (m, 4H), 4.91-5.02 (m, 4H), 5.76-5.86 (m, 2H). LCMS: Rt: 1.510 min; MS m/z (ESI): 972.8 [M+H]⁺.

The following compounds were prepared in analogous fashion as Compound 246, using corresponding starting material.

| Compound | Characterization |
|---|---|
| 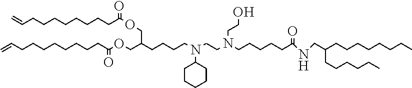 [SMILES: C=CCCCCCCCCC(=O)OCC(CCCCN(CCN(CCO)CCCCCC(=O)NCC(CCCCCC)CCCCCCCC)C1CCCCC1)COC(=O)CCCCCCCCC=C] Compound 250 | ¹H NMR (400 MHz, CDCl₃) δ: 0.86-0.89 (m, 6H), 1.26-1.39 (m, 55H), 1.59-1.68 (m, 10H), 1.96-2.06 (m, 9H), 2.14-2.20 (m, 7H), 2.28-2.32 (m, 5H), 2.58-2.66 (m, 2H), 2.73- 2.77 (m, 2H), 2.94-3.06 (m, 5H), 3.15-3.18 (m, 2H), 3.69 (s, 2H), 3.99-4.10 (m, 4H), 4.90-5.01 (m, 4H), 5.76-5.87 (m, 2H). LCMS: Rt: 1.360 min; MS m/z (ESI): 986.9 [M + H]⁺. |

### 6.78 Example 78: Preparation of Compound 247

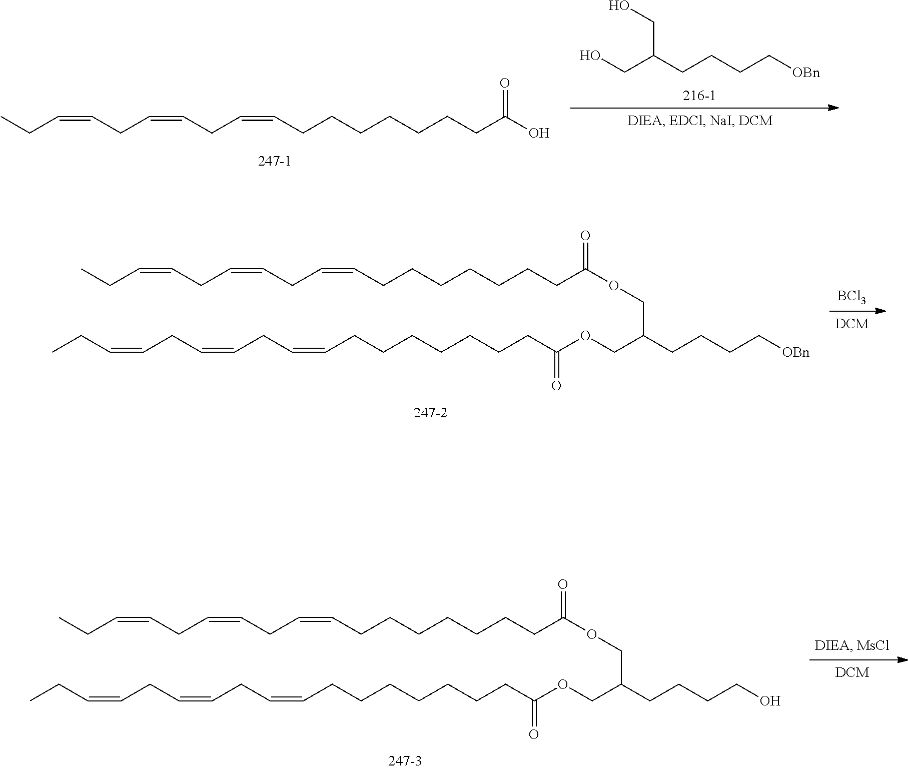

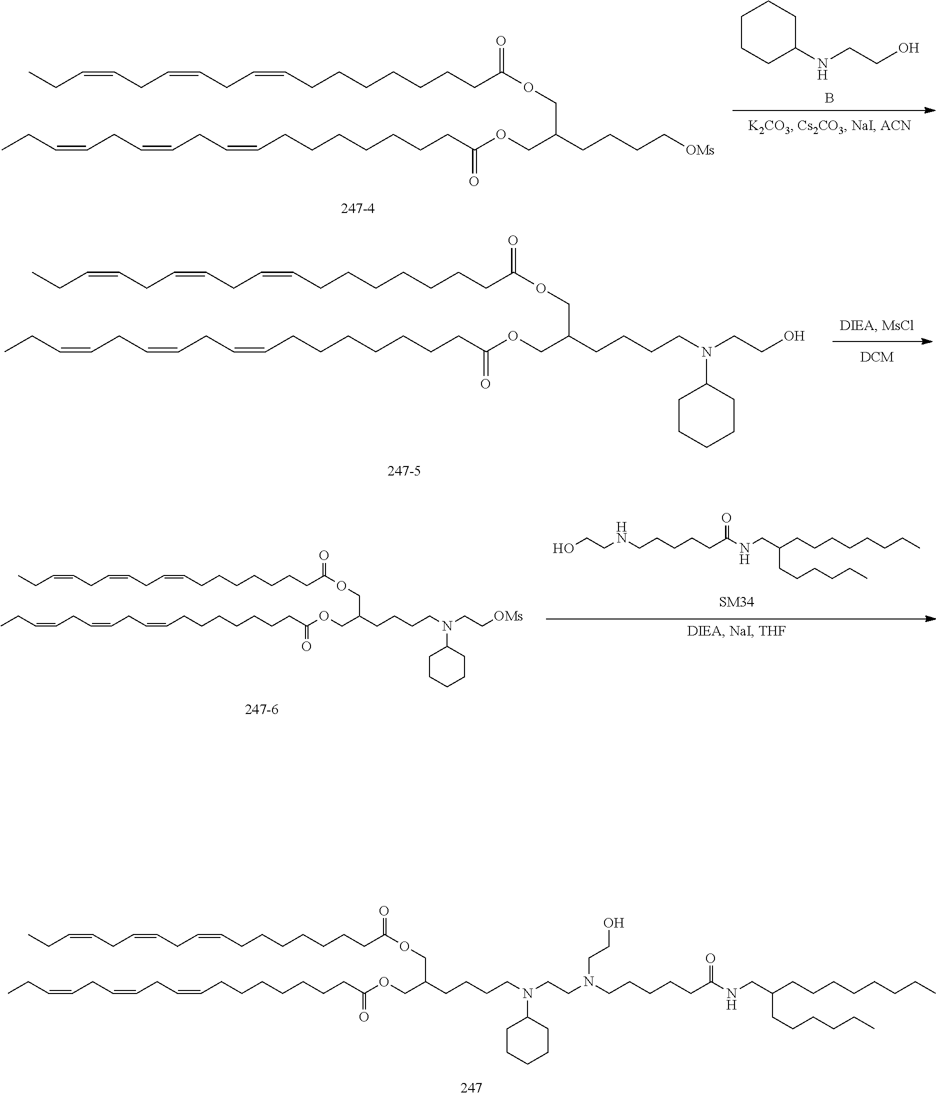

### Step 1: Preparation of Compound 247-2

To a solution of 247-1 (3.5 g, 12.6 mmol, 3.0 eq.) and compound 216-1 (1.0 g, 4.2 mmol, 1.0 eq.) in DCM (50 mL) was added DIEA (2.7 g, 21.0 mmol, 5.0 eq.), EDCI (2.4 g, 12.6 mmol, 3.0 eq.) and DMAP (1.3 g, 8.4 mmol, 2.0 eq.). The mixture was stirred at 50° C. for 16 hours. The reaction mixture was poured into water and extracted with DCM. The combined organic layers were washed with brine, dried over Na₂SO₄ and concentrated. The residue was purified by column chromatography on silica gel (PE/EA=10:1) to give compound 247-2 (2.8 g, 87.5% yield) as colorless oil. ¹H NMR (400 MHz, CDCl₃) δ: 0.87-1.03 (m, 6H), 1.25-1.43 (m, 20H), 1.58-1.65 (m, 6H), 2.02-2.11 (m, 9H), 2.24-2.34 (m, 4H), 2.69-2.90 (m, 8H), 3.45-3.51 (m, 2H), 4.02-4.10 (m, 4H), 4.49-4.52 (m, 2H), 4.24-4.54 (m, 12H), 4.27-4.38 (m, 5H).

### Step 2: Preparation of Compound 247-3

To a solution of 247-2 (1.0 g, 1.3 mmol, 1.0 eq.) in DCM (20 mL) was added BCl₃ (15.6 mL, 15.6 mmol, 12.0 eq.) at −78° C. The mixture was stirred at −78° C. for 1 hour. The mixture was poured into NaHCO₃ (aq) and extracted with DCM. The organic layer was washed with brine, dried over Na₂SO₄, concentrated and purified by column chromatography on silica gel (PE/EA=5:1) to give compound 247-3 (634 mg, 72.87% yield) as colorless oil. ¹H NMR (400 MHz, CDCl₃) δ: 0.90-1.00 (m, 6H), 1.26-1.37 (m, 20H), 1.58-1.63 (m, 4H), 1.70-1.80 (m, 2H), 2.03-2.12 (m, 8H), 2.26-2.34 (m, 5H), 2.45-2.55 (m, 1H), 2.77-2.87 (m, 6H), 3.63-3.66 (m, 2H), 3.78-3.94 (m, 1H), 4.02-4.11 (m, 5H), 5.25-5.60 (m, 12H).

### Step 3: Preparation of Compound 247-4

To a solution of 247-3 (630 mg, 0.94 mmol, 1.0 eq.) and DIPEA (243 mg, 1.88 mmol, 2.0 eq.) in DCM (20 mL) was added MsCl (129 mg, 1.13 mmol, 1.2 eq.) at 011. The mixture was stirred at room temperature for 2 hours. The mixture was poured into water and extracted with DCM. The combined organic layers were washed with brine, dried over Na₂SO₄ and concentrated to give compound 247-4 (652 mg, 92.88% yield) as yellow oil. It was used in the next step without further purification.

### Step 4: Preparation of Compound 247-5

To a solution of 247-4 (600 mg, 0.8 mmol, 1.0 eq.) and compound B (229 mg, 1.6 mmol, 2.0 eq.) in ACN (10 mL) were added K₂CO₃ (332 mg, 2.4 mmol, 3.0 eq.), Cs₂CO₃ (78 mg, 0.24 mmol, 0.23 eq.) and NaI (36 mg, 0.24 mmol, 0.3 eq.). The mixture was stirred at 80° C. for 16 hours. LCMS showed the reaction was complete. The reaction mixture was poured into water and extracted with EA. The combined organic layers were washed with brine, dried over Na₂SO₄ and concentrated. The residue was purified by column chromatography on silica gel (DCM/MeOH=20/1) to give compound 247-5 (472 mg, 74.33% yield) as yellow oil. LCMS: Rt: 1.135 min; MS m/z (ESI): 794.7 [M+H]⁺.

### Step 5: Preparation of Compound 247-6

To a solution of 247-5 (472 mg, 0.59 mmol. 1.0 eq.) and DIPEA (152 mg, 1.18 mmol, 2.0 eq.) in DCM (10 mL) was added MsCl (81 mg, 0.71 mmol, 1.2 eq.) at 011. The mixture was stirred at room temperature for 2 hours. The mixture was poured into water and extracted with DCM. The combined organic layers were washed with brine, dried over Na₂SO₄ and concentrated to give compound 247-6 (500 mg, 97.09% yield) as yellow oil. It was used in the next step without further purification.

### Step 6: Preparation of Compound 247

To a solution of 247-6 (500 mg, 0.6 mmol, 1.0 eq.) and compound SM34 (480 mg, 1.2 mmol, 2.0 eq.) in THF (10 mL) were added DIEA (230 mg, 1.8 mmol, 3.0 eq.) and NaI (30 mg, 0.18 mmol, 0.3 eq.). The mixture was stirred at 70° C. for 16 hours. LCMS showed the reaction was complete. The reaction mixture was poured into water and extracted with EA. The organic layer was washed with brine, dried over Na₂SO₄, concentrated and purified by prep-HPLC to give compound 247 (38 mg, 5.4% yield) as yellow oil.

¹H NMR (400 MHz, CDCl₃) δ: 0.86-0.91 (m, 6H), 0.94-1.01 (m, 6H), 0.26-1.33 (m, 50H), 1.57-1.69 (m, 10H), 1.76-1.84 (m, 4H), 1.89-2.06 (m, 12H), 2.10-2.21 (m, 3H), 2.26-2.32 (m, 6H), 2.53-2.67 (m, 6H), 2.70-2.85 (m, 9H), 3.15-3.23 (m, 3H), 3.47-3.74 (m, 3H), 4.00-4.15 (m, 5H), 5.28-5.45 (m, 12H). LCMS: Rt: 25.165 min; MS m/z (ESI): 1174.8 [M+H]⁺.

### 6.79 Example 79: Preparation of Compound 261

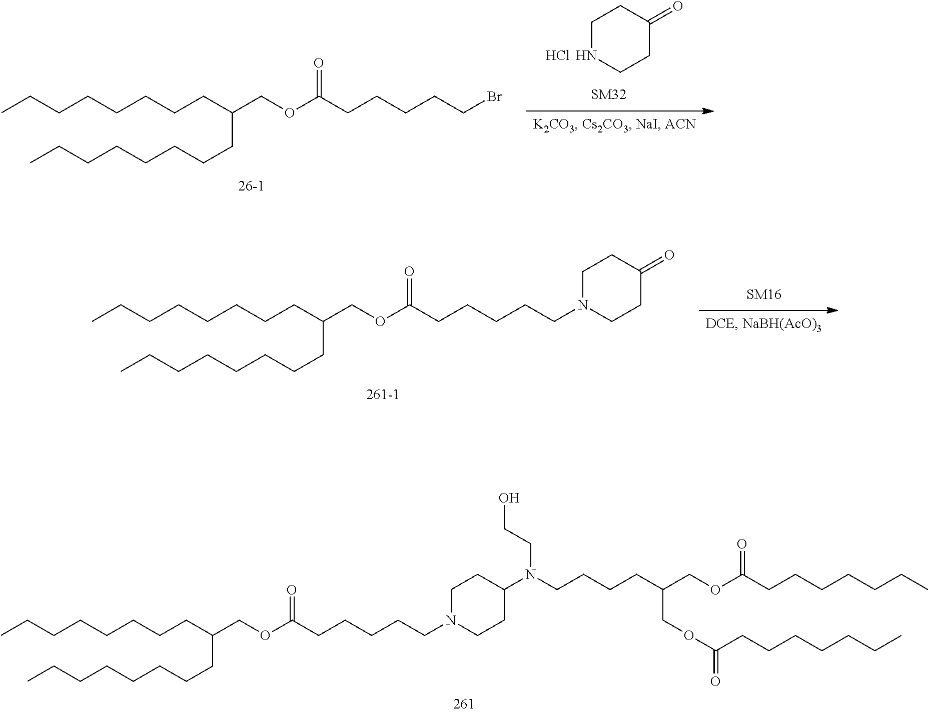

### Step 1: Preparation of Compound 261-1

To a solution of compound 26-1 (895 mg, 2.0 mmol, 1.0 eq.) and compound SM32 (407 mg, 3.0 mmol, 1.5 eq.) in ACN (20 mL) were added K₂CO₃ (829 mg, 6.0 mmol, 3.0 eq.), Cs₂CO₃ (195 mg, 0.6 mmol, 0.3 eq.) and NaI (90 mg, 0.6 mmol, 0.3 eq.). The mixture was stirred at 70° C. for 16 hours. LCMS showed the reaction was completed. The mixture was poured into water and extracted with EA. The combined organic layers were washed with brine, dried over Na₂SO₄ and concentrated. The residue was purified by column chromatography on silica gel (DCM/MeOH=40/1) to give compound 261-1 (530 mg, 57% yield) as yellow oil.

### Step 2: Preparation of Compound 261

To a solution of compound 261-1 (200 mg, 0.43 mmol, 1.0 eq.) and compound SM16 (191 mg, 0.43 mmol, 1.0 eq.) in DCE (8 mL) were added AcOH (1 drop). The mixture was stirred at rt for 6 hours. Then NaBH(OAc)₃ (137 mg, 0.65 mmol, 1.5 eq.) was added. The mixture was stirred at rt for 16 hours. LCMS showed the reaction was completed. The mixture was poured into water and extracted with EA. The combined organic layers were washed with brine, dried over Na₂SO₄ and concentrated. The residue was purified by prep-HPLC to give compound 261 (22 mg, 6% yield) as yellow oil.

¹H NMR (400 MHz, CDCl₃) δ: 0.86-0.90 (m, 12H), 1.26-1.41 (m, 53H), 1.56-1.69 (m, 18H), 1.92-2.03 (m, 2H), 2.29-2.32 (m, 7H), 2.52-2.90 (m, 6H), 3.96-4.10 (m, 6H). LCMS: Rt: 0.520 min; MS m/z (ESI): 893.6 [M+H]⁺.

### 6.80 Example 80: Preparation and Characterization of Lipid Nanoparticles

Briefly, a cationic lipid provided herein, DSPC, cholesterol, and PEG-lipid were solubilized in ethanol at a molar ratio of 50:10:38.5:1.5, and mRNA were diluted in 10 to 50 mM citrate buffer, pH=4. The LNPs were prepared at a total lipid to mRNA weight ratio of approximately 10:1 to 30:1 by mixing the ethanolic lipid solution with the aqueous mRNA solution at a volume ratio of 1:3 using a microfluidic apparatus, total flow rate ranging from 9-30 mL/min. Ethanol were thereby removed and replaced by DPBS using dialysis. Finally, the lipid nanoparticles were filtered through a 0.2 m sterile filter.

Lipid nanoparticle size were determined by dynamic light scattering using a Malvern Zetasizer Nano ZS (Malvern UK) using a 1730 backscatter detection mode. The encapsulation efficiency of lipid nanoparticles were determined using a Quant-it Ribogreen RNA quantification assay kit (Thermo Fisher Scientific, UK) according to the manufacturer's instructions.

As reported in literature, the apparent pKa of LNP formulations correlates with the delivery efficiency of LNPs for nucleic acids in vivo. The apparent pKa of each formulation was determined using an assay based on fluorescence of 2-(p-toluidino)-6-napthalene sulfonic acid (TNS). LNP formulations comprising of cationic lipid/DSPC/cholesterol/DMG-PEG (50/10/38.5/1.5 mol %) in PBS were prepared as described above. TNS was prepared as a 300 μM stock solution in distilled water. LNP formulations were diluted to 0.1 mg/mL total lipid in 3 mL of buffered solutions containing 50 mM sodium citrate, 50 mM sodium phosphate, 50 mM sodium borate, and 30 mM sodium chloride where the pH ranged from 3 to 9. An aliquot of the TNS solution was added to give a final concentration of 0.1 mg/mL and following vortex mixing fluorescence intensity was measured at room temperature in a Molecular Devices Spectramax iD3 spectrometer using excitation and mission wavelengths of 325 nm and 435 nm. A sigmoidal best fit analysis was applied to the fluorescence data and the pKa value was measured as the pH giving rise to half-maximal fluorescent intensity.

### 6.81 Example 81: Animal Study

Lipid nanoparticles comprising compounds in the following table encapsulating human erythropoietin (hEPO) mRNA were systemically administered to 6-8 week old female ICR mice (Xipuer-Bikai, Shanghai) at 0.5 mg/kg dose by tail vein injection and mice blood were sampled at specific time points (e.g., 6 hours) post administration. In addition to the aforementioned tested groups, lipid nanoparticles comprising dilinoleylmethyl-4-dimethylaminobutyrate (DL in-MC3-DMA, usually abbreviated to MC3) encapsulating hEPO mRNA were similarly administered at the same dose to age and gender comparative groups of mice as a positive control.

Mice were euthanized by CO₂ overdoses after the last sampling time point. Serum were separated from total blood by centrifugation at 5000 g for 10 minutes at 4° C., snap-frozen and stored at −80° C. for analysis. ELISA assay were carried out using a commercial kit (DEP00, R&D systems) according to manufacturer's instructions.

Characteristics of tested lipid nanoparticles, including expression levels over MC3 measured from the tested group are listed in the table below.

TABLE 2

| | size | poly- | Encapsulation | Expression | Apparent |
|---|---|---|---|---|---|
| Lipid | (nm) | dispersity | Efficiency | over MC3 | Pka |
| 1 | 58.78 | 0.158 | 93.1% | B | 6.374 |
| 2 | 64.03 | 0.066 | 86.6% | D | |
| 3 | 73.33 | 0.131 | 93.6% | C | 6.32 |
| 4 | 90.8 | 0.076 | 91.9% | C | 6.709 |
| 5 | 99.12 | 0.073 | 92.7% | C | 6.56 |
| 6 | 101.6 | 0.089 | 87.7% | C | |
| 7 | 72.78 | 0.06 | 96.5% | B | 6.427 |
| 8 | 65.15 | 0.136 | 95.8% | C | 6.401 |
| 9 | 55.12 | 0.181 | 93.9% | C | |
| 10 | 70.21 | 0.168 | 86.6% | A | 6.176 |
| 11 | 66.15 | 0.055 | 82.8% | A | |
| 12 | 58.17 | 0.062 | 89.5% | A | 6.685 |
| 13 | 60.71 | 0.105 | 83.3% | A | 6.633 |
| 14 | 60.06 | 0.118 | 89.8% | A | |
| 15 | 65.95 | 0.022 | 86.0% | A | 6.724 |
| 16 | 59.48 | 0.035 | 99.2% | A | 6.757 |
| 17 | 58.8 | 0.033 | 98.9% | A | 6.671 |
| 18 | 56.64 | 0.049 | 96.4% | C | 6.018 |
| 19 | 80.6 | 0.046 | 90.2% | A | 6.269 |
| 20 | 74.98 | 0.015 | 95.8% | C | |
| 21 | 81.93 | 0.042 | 92.3% | A | 6.361 |
| 22 | 87.7 | 0.047 | 95.5% | A | |
| 23 | 57.5 | 0.039 | 95.5% | A | 6.686 |
| 24 | 57.78 | 0.114 | 93.90% | A | 6.162 |
| 25 | 50.59 | 0.068 | 90.67% | D | 4.853 |
| 26 | 63.47 | 0.13 | 97.57% | C | 7.024 |
| 27 | 57.08 | 0.118 | 95.20% | C | 6.858 |
| 28 | 62.35 | 0.045 | 92.4% | A | 6.686 |
| 29 | 59.02 | 0.09 | 93.5% | C | 6.073 |
| 30 | 53.56 | 0.115 | 92.9% | B | 6.131 |
| 31 | 54.55 | 0.092 | 88.2% | C | 5.708 |
| 32 | 57.09 | 0.04 | 92.1% | A | 6.378 |
| 33 | 57.51 | 0.052 | 87.6% | C | 6.305 |
| 34 | 55.14 | 0.05 | 87.2% | D | 5.485 |
| 35 | 54.18 | 0.089 | 90.0% | C | 5.843 |
| 36 | 49.69 | 0.093 | 89.3% | C | 5.802 |
| 37 | 71.53 | 0.037 | 87.2% | B | 6.988 |
| 38 | 54.42 | 0.092 | 89.8% | D | 6.253 |
| 39 | 55.02 | 0.034 | 84.0% | C | 5.740 |
| 40 | 52.06 | 0.03 | 87.1% | C | 5.838 |
| 41 | 55.24 | 0.067 | 88.2% | B | 6.134 |
| 42 | 66.04 | 0.046 | 90.1% | A | 6.159 |
| 43 | 66.41 | 0.07 | 89.6% | A | 6.346 |
| 44 | 77.48 | 0.081 | 92.6% | A | |
| 45 | 63.5 | 0.051 | 90.9% | A | 6.402 |
| 46 | 64.14 | 0.042 | 90.3% | A | 6.335 |
| 47 | 66.29 | 0.037 | 91.0% | A | 6.298 |
| 48 | 73.5 | 0.037 | 92.1% | A | 6.295 |
| 49 | 55.04 | 0.1 | 95.5% | C | 6.153 |
| 50 | 59.59 | 0.235 | 93.4% | C | 5.786 |
| 51 | 83.92 | 0.028 | 97.0% | C | 6.579 |
| 52 | 67.63 | 0.089 | 95.6% | B | 6.114 |
| 53 | 63.7 | 0.062 | 95.7% | C | 5.872 |
| 54 | 77.99 | 0.065 | 93.4% | D | 6.51 |
| 55 | 83.97 | 0.071 | 95.6% | B | 5.951 |
| 56 | 57.44 | 0.174 | 92.96% | C | 6.702 |
| 57 | 79.95 | 0.044 | 91.7% | B | 6.509 |
| 58 | 110 | 0.1 | 97.7% | B | 7.327 |
| 59 | 70.3 | 0.063 | 95.1% | A | |
| 60 | 59.89 | 0.039 | 91.7% | C | 6.761 |
| 61 | 66.08 | 0.026 | 95.4% | A | 6.731 |
| 62 | 68.28 | 0.034 | 92.0% | A | 6.21 |
| 63 | 87.84 | 0.044 | 93.93% | C | 6.147 |
| 64 | 81.36 | 0.177 | 97.95% | D | 7.262 |
| 65 | 104.2 | 0.044 | 93.10% | C | 6.443 |
| 66 | 56.3 | 0.068 | 93.17% | C | 6.009 |
| 67 | 75.57 | 0.023 | 95.1% | B | 5.980 |
| 68 | 54.78 | 0.119 | 90.31% | D | 5.576 |
| 69 | 60.99 | 0.096 | 88.86% | C | |
| 70 | 126 | 0.005 | 91.32% | B | 6.732 |
| 71 | 65.56 | 0.091 | 89.25% | A | 6.489 |
| 72 | 74.18 | 0.033 | 92.60% | A | 6.633 |
| 73 | 88.45 | 0.049 | 92.38% | A | 6.597 |
| 74 | 67.96 | 0.05 | 92.74% | A | |
| 75 | 76.62 | 0.037 | 92.59% | A | |
| 76 | 84.12 | 0.036 | 92.30% | C | 6.322 |
| 77 | 84.87 | 0.006 | 92.71% | A | 6.485 |
| 78 | 100.3 | 0.019 | 88.18% | D | 6.195 |
| 79 | 69.09 | 0.104 | 96.26% | C | 6.914 |
| 80 | 90.40 | 0.137 | 97.13% | C | |
| 81 | 55.27 | 0.051 | 91.82% | C | 6.298 |
| 82 | 67.32 | 0.066 | 91.54% | D | 6.599 |
| 83 | 87.87 | 0.032 | 92.51% | B | 6.233 |
| 84 | 59.91 | 0.097 | 91.88% | C | 6.117 |
| 85 | 67.44 | 0.096 | 93.27% | C | 6.126 |
| 86 | 86.61 | 0.043 | 95.02% | A | 6.814 |
| 87 | 70.07 | 0.055 | 93.34% | C | 6.476 |
| 88 | 71.00 | 0.080 | 92.56% | D | |
| 89 | 81.46 | 0.086 | 89.05% | C | 6.745 |
| 90 | 101.70 | 0.030 | 82.56% | C | |
| 91 | 64.49 | 0.115 | 87.92% | D | 6.042 |
| 92 | 80.12 | 0.014 | 91.34% | C | 6.582 |
| 93 | 76.12 | 0.064 | 92.84% | C | |
| 94 | 58.34 | 0.061 | 90.26% | C | 6.448 |
| 95 | 55.48 | 0.103 | 92.04% | C | 6.271 |
| 96 | 57.20 | 0.087 | 97.00% | C | 7.287 |
| 97 | 76.13 | 0.045 | 88.12% | C | 6.912 |
| 98 | 91.85 | 0.042 | 89.31% | B | |
| 99 | 87.73 | 0.135 | 83.64% | A | |
| 100 | 93.16 | 0.085 | 85.49% | C | |
| 101 | 98.67 | 0.078 | 87.20% | B | 6.423 |
| 102 | 77.17 | 0.082 | 90.14% | B | 6.407 |
| 103 | 73.64 | 0.066 | 92.75% | C | 6.319 |
| 104 | 66.62 | 0.044 | 92.43% | C | |
| 105 | 69.11 | 0.051 | 93.35% | C | 6.328 |
| 106 | 67.63 | 0.067 | 92.14% | C | |
| 107 | 55.62 | 0.121 | 93.22% | D | 6.411 |
| 108 | 67.69 | 0.068 | 91.83% | C | |
| 109 | 78.27 | 0.032 | 92.54% | C | 6.348 |
| 110 | 84.07 | 0.042 | 93.11% | A | 6.293 |
| 111 | 76.24 | 0.069 | 92.05% | A | 6.108 |
| 112 | 77.33 | 0.059 | 93.15% | A | 6.343 |
| 113 | 89.42 | 0.012 | 92.93% | A | 6.364 |
| 114 | 81.25 | 0.044 | 90.35% | C | |
| 115 | 97.51 | 0.058 | 90.22% | C | |
| 116 | 77.45 | 0.102 | 87.45% | D | 6.826 |
| 117 | 79.67 | 0.050 | 89.34% | D | |
| 118 | 57.96 | 0.121 | 94.10% | B | 6.044 |
| 119 | 69.75 | 0.087 | 92.21% | B | |
| 120 | 75.42 | 0.043 | 94.33% | A | 6.546 |
| 121 | 70.01 | 0.077 | 93.23% | A | 6.583 |
| 122 | 65.77 | 0.066 | 93.47% | A | 6.817 |
| 123 | 59.28 | 0.152 | 94.58% | A | 6.336 |
| 124 | 59.55 | 0.111 | 94.60% | B | 5.997 |
| 125 | 90.98 | 0.035 | 92.89% | C | |
| 126 | 61.57 | 0.047 | 92.26% | B | 6.212 |
| 127 | 69.05 | 0.090 | 91.77% | A | 6.226 |
| 128 | 60.57 | 0.093 | 92.31% | D | 6.539 |
| 129 | 58.08 | 0.069 | 90.52% | C | |
| 130 | 76.86 | 0.060 | 91.06% | B | |
| 131 | 56.62 | 0.121 | 92.18% | B | 6.255 |
| 133 | 74.57 | 0.267 | 94.56% | C | 5.990 |
| 135 | 124.60 | 0.035 | 87.16% | B | |
| 136 | 84.74 | 0.040 | 88.64% | A | 6.831 |
| 137 | 67.32 | 0.040 | 89.36% | A | 6.494 |
| 138 | 81.24 | 0.107 | 90.03% | B | |
| 139 | 59.97 | 0.076 | 87.81% | B | |
| 140 | 71.01 | 0.051 | 89.74% | A | |
| 141 | 63.69 | 0.128 | 88.11% | B | 6.475 |
| 142 | 71.85 | 0.055 | 87.29% | A | 6.218 |
| 143 | 69.16 | 0.057 | 90.39% | A | 6.687 |
| 144 | 68.28 | 0.099 | 84.58% | A | 6.182 |
| 145 | 68.91 | 0.047 | 88.63% | A | |
| 147 | 85.76 | 0.058 | 88.17% | A | 6.62 |
| 148 | 64.31 | 0.081 | 90.40% | A | |
| 149 | 78.75 | 0.043 | 92.71% | A | |
| 150 | 76.37 | 0.076 | 91.34% | B | 6.586 |
| 151 | 61.84 | 0.064 | 92.43% | B | 6.368 |
| 152 | 83.28 | 0.031 | 91.68% | A | 6.646 |
| 153 | 73.95 | 0.048 | 93.09% | A | 6.437 |
| 154 | 56.95 | 0.101 | 87.11% | C | 6.061 |
| 155 | 70.67 | 0.071 | 89.57% | A | 6.28 |
| 156 | 73.36 | 0.190 | 89.69% | C | |
| 157 | 88.09 | 0.112 | 95.04% | B | 6.674 |
| 158 | 63.54 | 0.058 | 89.17% | D | |
| 159 | 77.72 | 0.080 | 90.71% | D | |
| 160 | 86.16 | 0.096 | 86.18% | A | |
| 161 | 72.08 | 0.090 | 87.54% | B | 6.02 |
| 162 | 82.68 | 0.068 | 85.46% | A | |
| 165 | 131.30 | 0.125 | 75.66% | C | 6.183 |
| 166 | 75.12 | 0.092 | 92.85% | B | 6.513 |
| 167 | 69.47 | 0.128 | 91.97% | B | |
| 168 | 78.08 | 0.107 | 91.48% | C | 6.537 |
| 169 | 73.05 | 0.153 | 89.88% | | 6.447 |
| 170 | 74.62 | 0.09 | 96.0% | A | 6.269 |
| 171 | 76.26 | 0.076 | 96.23% | C | |
| 172 | 69.59 | 0.061 | 93.55% | C | |
| 173 | 61.61 | 0.068 | 95.29% | C | |
| 174 | 78.27 | 0.063 | 91.79% | C | |
| 175 | 97.65 | 0.079 | 89.31% | C | 6.97 |
| 176 | 65.70 | 0.123 | 95.27% | B | |
| 177 | 95.00 | 0.059 | 93.69% | C | 6.668 |
| 178 | 96.32 | 0.056 | 89.15% | A | 6.337 |
| 179 | 103.40 | 0.041 | 89.97% | A | 6.322 |
| 180 | 80.36 | 0.095 | 91.55% | B | 6.699 |
| 181 | 119.3 | 0.154 | 94.10% | B | 6.995 |
| 182 | 99.97 | 0.09 | 91.00% | B | 6.862 |
| 183 | 103.2 | 0.032 | 92.26% | C | 6.858 |
| 184 | 129.75 | 0.172 | 90.65% | C | 7.098 |
| 185 | 75.27 | 0.111 | 90.39% | C | 6.593 |
| 186 | 72.88 | 0.139 | 91.69% | C | 7.617 |
| 187 | 88.82 | 0.083 | 94.26% | B | 6.226 |
| 188 | 111.8 | 0.085 | 86.80% | D | 6.738 |
| 189 | 87.22 | 0.114 | 93.88% | C | 6.542 |
| 190 | 78.4 | 0.13 | 88.81% | A | 6.699 |
| 191 | 86.61 | 0.117 | 93.35% | B | 6.552 |
| 192 | 82.66 | 0.109 | 90.40% | B | 6.707 |
| 193 | 80.39 | 0.228 | 84.30% | A | 6.666 |
| 194 | 102.1 | 0.15 | 84.59% | | 6.705 |
| 195 | 104.2 | 0.153 | 83.82% | B | 6.925 |
| 196 | 104.1 | 0.18 | 86.33% | D | |
| 197 | 70.01 | 0.158 | 91.45% | C | |
| 198 | 96.11 | 0.069 | 88.41% | A | 6.984 |
| 199 | 80.14 | 0.162 | 94.30% | A | 6.858 |
| 200 | 84.18 | 0.054 | 92.81% | C | 6.593 |
| 201 | 91.64 | 0.028 | 94.15% | A | 6.226 |
| 202 | 84.35 | 0.042 | 88.47% | A | 6.738 |
| 203 | 131.9 | 0.16 | 78.17% | | 6.874 |
| 205 | 104.3 | 0.023 | 81.79% | B | 6.917 |
| 206 | 112.7 | 0.07 | 80.88% | B | 6.81 |
| 207 | 87.97 | 0.071 | 87.94% | A | 6.657 |
| 208 | 85.95 | 0.053 | 86.63% | A | 6.691 |
| 209 | 95.78 | 0.039 | 87.79% | A | 6.608 |
| 210 | 79.56 | 0.06 | 91.52% | A | 6.49 |
| 211 | 112.6 | 0.035 | 84.88% | B | |
| 212 | 122.1 | 0.048 | 87.31% | C | |
| 213 | 110.8 | 0.07 | 88.38% | C | |
| 214 | 76.29 | 0.058 | 89.14% | A | 6.789 |
| 215 | 67.78 | 0.033 | 90.80% | C | 6.542 |
| 218 | 82.04 | 0.043 | 93.64% | C | |
| 219 | 91.17 | 0.103 | 94.13% | C | 6.327 |
| 220 | 94.56 | 0.15 | 94.84% | B | |
| 221 | 99.33 | 0.07 | 93.71% | C | 6.316 |
| 222 | 121 | 0.083 | 87.16% | C | |
| 223 | 70.98 | 0.043 | 93.03% | A | 6.395 |
| 224 | 104.1 | 0.027 | 85.65% | B | 6.748 |
| 225 | 76.81 | 0.033 | 92.20% | A | 6.668 |
| 227 | 83.54 | 0.048 | 91.70% | B | 6.872 |
| 229 | 77.73 | 0.042 | 92.04% | A | 6.849 |
| 230 | 89.46 | 0.051 | 89.65% | A | 6.742 |
| 231 | 73.66 | 0.036 | 92.25% | A | 6.712 |
| 232 | 77.78 | 0.031 | 92.47% | C | 6.567 |
| 233 | 76.91 | 0.052 | 91.15% | A | 6.463 |
| 234 | 67.07 | 0.035 | 88.68% | B | 6.566 |
| 235 | 67.92 | 0.093 | 88.53% | B | 6.545 |
| 236 | 86.17 | 0.051 | 91.34% | B | 6.43 |
| 237 | 77.85 | 0.033 | 88.77% | A | 6.651 |
| 261 | 82.86 | 0.066 | 95.18% | A | 6.552 |

A: ≥2
B: ≥1 and <2
C: ≥0.1 and <1
D: <0.1

### 6.82 Example 82: Lipid Clearance Study

LNPs were injected into mice via the tail vein (ICR female, IV, 0.5 mg mRNA/kg), then mice were anesthetized under carbon dioxide at different time (e.g., 6 h, 24 h, and 48 h) after administration and sacrificed via cardiac puncture. Liver tissue were collected immediately, and then washed by ice-cold saline. Liver samples were weighed and homogenized in ice water bath by adding pre-chilled 20% methanol-water (v/v) at 2˜8° C. with the ratio of 1:5 (w/v). The homogenized tissue samples were stored in −90˜−60° C. freezer prior to analysis.

Sample Processing. All liver tissue homogenate samples were allowed to thaw at room temperature. An aliquot of 50 μL of sample was added with 50 μL MgCl₂ (2M), then added with ACN which contains of Verapamil, 5 ng·mL⁻¹ and Glibenclamide, 50 ng·mL⁻¹ and Diclofenac, 200 ng·mL¹ and Tolbutamide, 200 ng·mL¹ for protein precipitation, then centrifuged at 13000 rpm for 8 min. Then 100 μL of supernatant was added with 100 μL water, then vortexed well. An aliquot of 5 μL of the mixture was injected into the LC-MS/MS system.

The results of MC3 and selected lipid compounds provided herein are listed in the table below.

TABLE 3

| | % Dose Remaining in Liver Tissue^{a} | | |
|---|---|---|---|
| Lipid | 6 h | 24 h | 48 h |
| MC3 | 69.39% | 61.63% | 52.47% |
| 74 | 48.44% | 50.42% | 50.83% |
| 71 | 1.34% | 0.00% | 0.00% |
| 99 | 22.45% | 16.39% | 14.08% |
| 21 | 94.55% | 67.66% | 24.38% |
| 218 | 7.38% | 1.03% | 0.17% |
| 220 | 7.87% | 1.98% | 0.52% |

^{a}Percent of original lipid dose in mouse liver at different time after 0.5 mg/kg i.v. bolus dose mRNA

1. A compound of Formula (I): 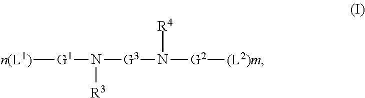
or a pharmaceutically acceptable salt, prodrug or stereoisomer thereof, wherein:
G¹ and G² are each independently a bond, C₂-C₁₂ alkylene, or C₂-C₁₂ alkenylene, wherein one or more —CH₂— in G¹ and G² is optionally replaced by —O—;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —NR^{a}P(═O)(OR^{b})(OR^{c}), —(C₆-C₁₀ arylene)-R¹, -(6- to 10-membered heteroarylene)-R¹, -(4- to 8-membered heterocyclylene)-R¹, or R¹;
each L² is independently —OC(═O)R², —C(═O)OR², —OC(═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR², —SC(═S)R², —C(═S)SR², —C(═S)R², —CH(OH)R², —P(═O)(OR^{e})(OR^{f}), —NR^{d}P(═O)(OR^{e})(OR^{f}), —(C₆-C₁₀ arylene)-R², -(6- to 10-membered heteroarylene)-R², -(4- to 8-membered heterocyclylene)-R², or R²;
R¹ and R² are each independently C₆-C₂₄ alkyl or C₆-C₂₄ alkenyl;
R^{a}, R^{b}, R^{d}, and R^{e} are each independently H, C₁-C₂₄ alkyl, or C₂-C₂₄ alkenyl;
R^{c} and R^{f} are each independently C₁-C₂₄ alkyl or C₂-C₂₄ alkenyl;
G³ is C₂-C₁₂ alkylene or C₂-C₁₂ alkenylene, wherein part or all of alkylene or alkenylene is optionally replaced by C₃-C₈ cycloalkylene, C₃-C₈ cycloalkenylene, C₃-C₈ cycloalkynylene, 4- to 8-membered heterocyclylene, C₆-C₁₀ arylene, or 5- to 10-membered heteroarylene;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety; or R³, G³ or part of G³, together with the nitrogen to which they are attached form a cyclic moiety;
R⁴ is C₁-C₁₂ alkyl or C₃-C₈ cycloalkyl;
x is 0, 1, or 2;
n is 1 or 2;
m is 1 or 2; and
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

2. The compound of claim 1, which is a compound of Formula (II-A), (II-B), (II-C), or (II-D): 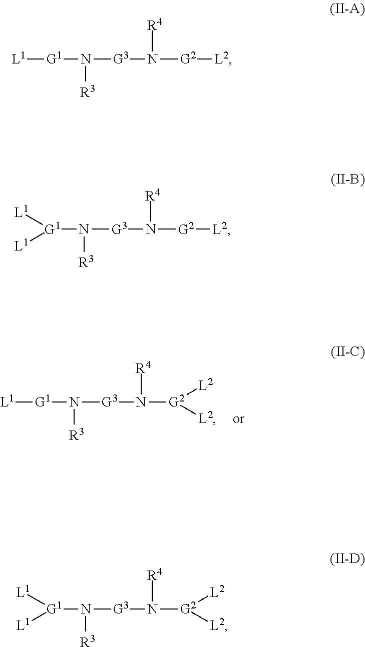
or a pharmaceutically acceptable salt, prodrug or stereoisomer thereof.

3. (canceled)

4. The compound of claim 1, which is a compound of Formula (III-A), (III-B), (III-C), or (III-D): 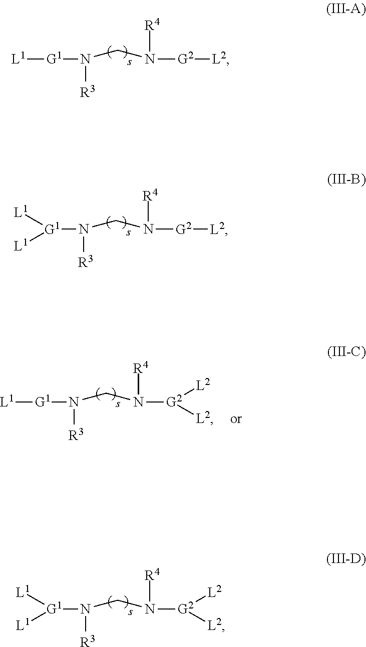
wherein s is an integer from 2 to 12,
or a pharmaceutically acceptable salt, prodrug or stereoisomer thereof.

5-7. (canceled)

8. The compound of claim 1, which is a compound of Formula (IV): 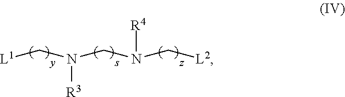
wherein s is an integer from 2 to 12,
y is an integer from 2 to 12; and
z is an integer from 2 to 12;
or a pharmaceutically acceptable salt, prodrug or stereoisomer thereof.

9. (canceled)

10. The compound of claim 8, which is a compound of Formula (IV-A), (IV-B3), (IV-C), (IV-D), (IV-E), (IV-F), (IV-G), or (IV-H): 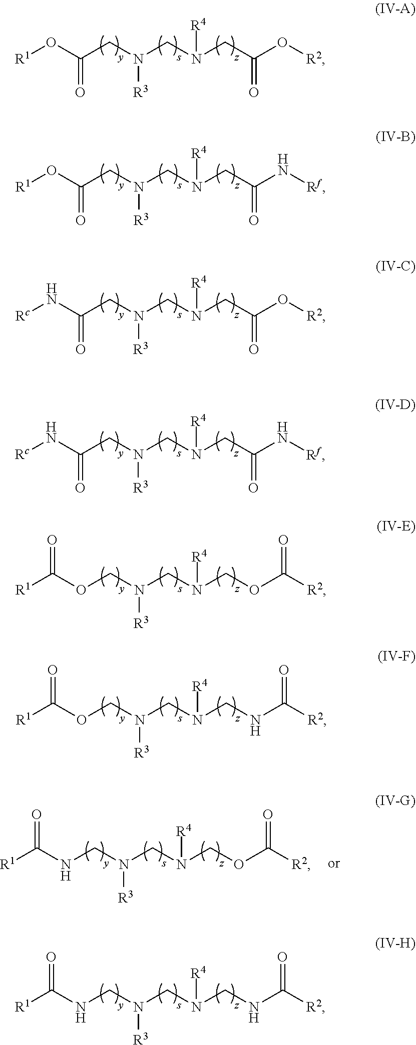
or a pharmaceutically acceptable salt, prodrug or stereoisomer thereof.

11. The compound of claim 1, which is a compound of Formula (V): 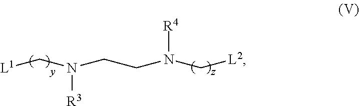
wherein y is an integer from 2 to 12; and
z is an integer from 2 to 12;
or a pharmaceutically acceptable salt, prodrug or stereoisomer thereof.

12. (canceled)

13. The compound of claim 11, which is a compound of Formula (V-A), (V-B), (V-C), (V-D), (V-E), (V-F), (V-G), or (V-H): 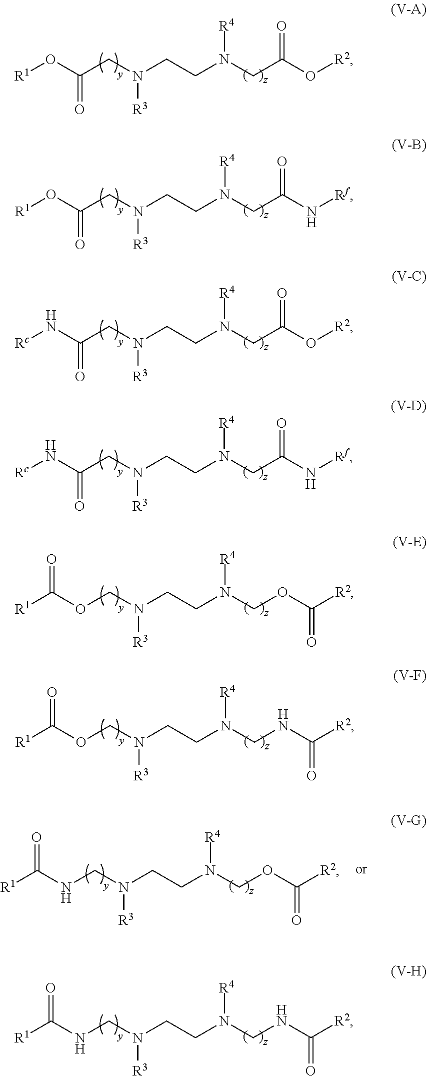
or a pharmaceutically acceptable salt, prodrug or stereoisomer thereof.

14. (canceled)

15. The compound of claim 1, which is a compound of Formula (VI): 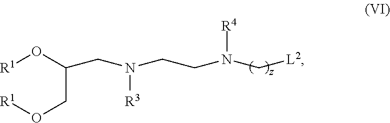
wherein z is an integer from 2 to 12;
or a pharmaceutically acceptable salt, prodrug or stereoisomer thereof.

16. The compound of claim 1, wherein R³ is C₁-C₁₂ alkyl, C₂-C₁₂ alkenyl, or C₃-C₈ cycloalkyl.

17. The compound of claim 1, wherein R³, G¹ or part of G¹, together with the nitrogen to which they are attached form a cyclic moiety.

18. The compound of claim 17, which is a compound of Formula (VII): 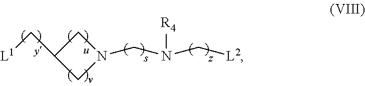
wherein s is an integer from 2 to 12,
u is 1, 2, or 3;
v is 1, 2, or 3;
y′ is an integer from 0 to 10; and
z is an integer from 2 to 12;
or a pharmaceutically acceptable salt, prodrug or stereoisomer thereof.

19. The compound of claim 1, wherein R³, G³ or part of G³, together with the nitrogen to which they are attached form a cyclic moiety.

20. The compound of claim 19, which is a compound of Formula (VIII-A), (VIII-B), (VIII-C), (VIII-D), (VIII-E), (VIII-F), or (VIII-G): 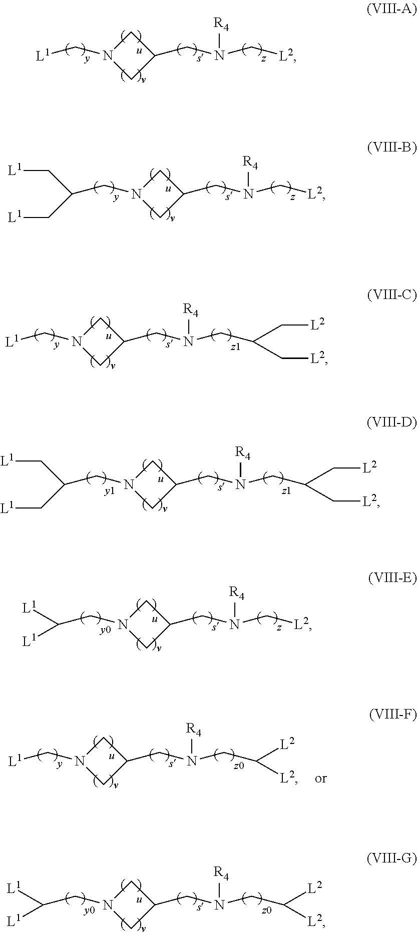
wherein s′ is an integer from 0 to 10,
u is 1, 2, or 3;
v is 1, 2, or 3;
y is an integer from 2 to 12;
z is an integer from 2 to 12;
y0 is an integer from 1 to 11;
z0 is an integer from 1 to 11;
y1 is an integer from 0 to 9; and
z1 is an integer from 0 to 9;
or a pharmaceutically acceptable salt, prodrug or stereoisomer thereof.

21. The compound of claim 1, which is a compound of Formula (IX-A), (IX-B), (IX-C), (IX-D), (IX-E), (IX-F), (IX-G), (IX-H), (IX-I), (IX-J), (IX-K), (IX-L), (IX-M), (IX-N), (IX-O), (IX-P), (IX-Q), (IX-R), (IX-S), (IX-T), (IX-U), (IX-V), (IX-W), (IX-X), (IX-Y), (IX-Z), or (IX-AA): 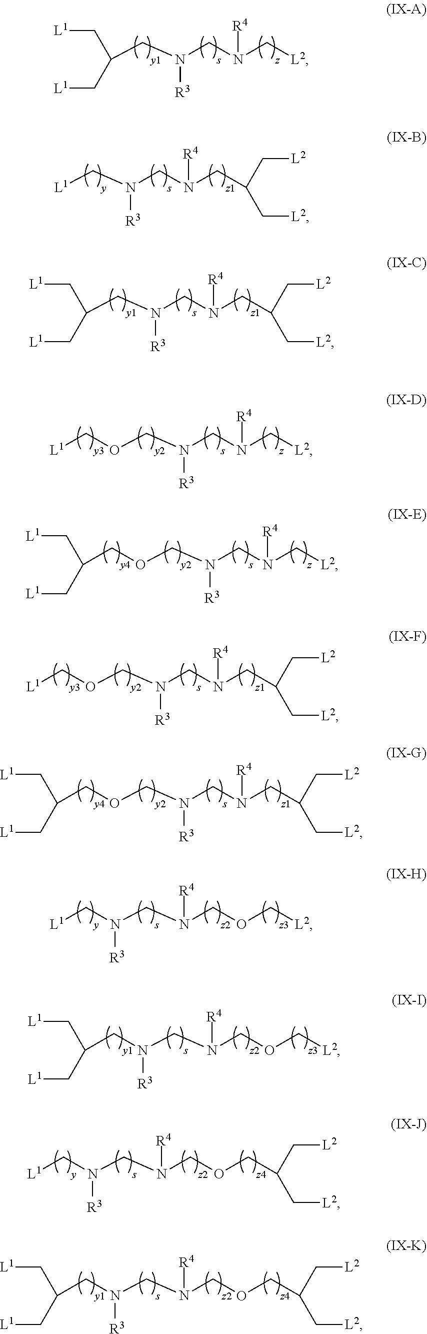 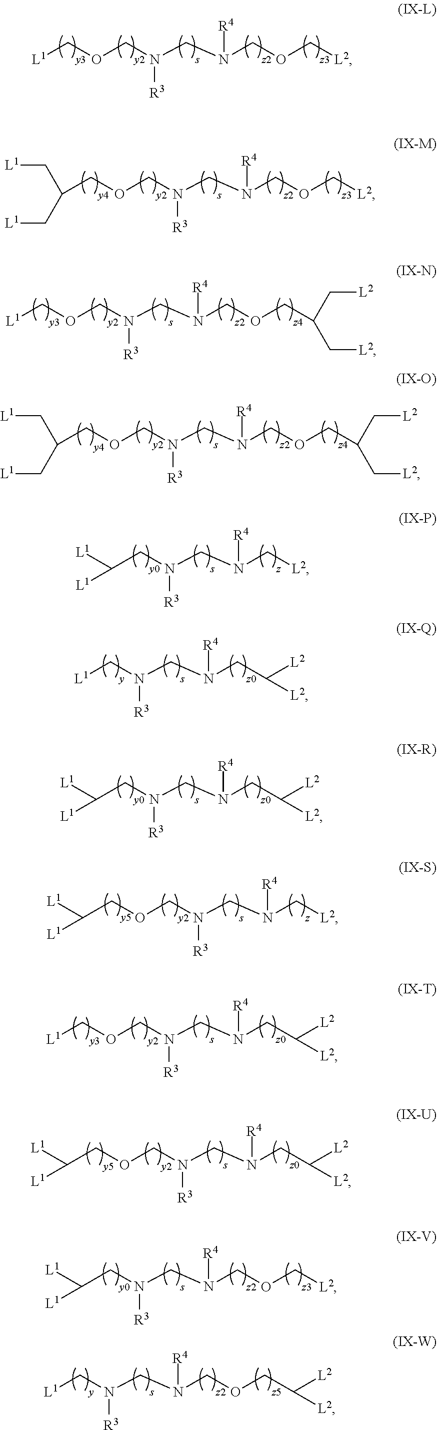 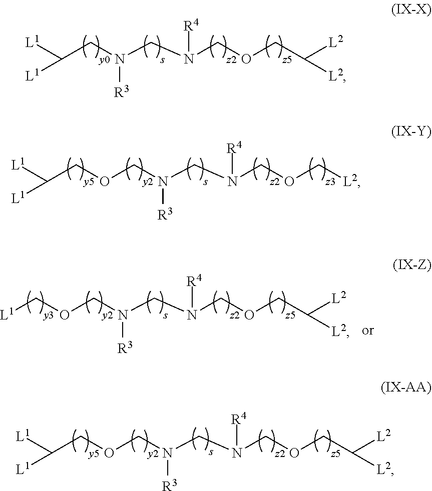
wherein s is an integer from 2 to 12,
y is an integer from 2 to 12;
z is an integer from 2 to 12;
y0 is an integer from 1 to 11;
z0 is an integer from 1 to 11;
y1 is an integer from 0 to 9;
z1 is an integer from 0 to 9;
y2 is an integer from 2 to 5;
y3 is an integer from 2 to 6;
y4 is an integer from 0 to 3;
y5 is an integer from 1 to 5;
z2 is an integer from 2 to 5;
z3 is an integer from 2 to 6;
z4 is an integer from 0 to 3; and
z5 is an integer from 1 to 5;
or a pharmaceutically acceptable salt, prodrug or stereoisomer thereof.

22-29. (canceled)

30. A compound in Table 1 or Table 1A, or a pharmaceutically acceptable salt, prodrug or stereoisomer thereof.

31. A compound of Formula (X): 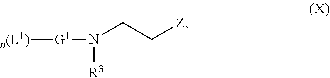
or a pharmaceutically acceptable salt, prodrug or stereoisomer thereof, wherein:
G¹ is a bond, C₂-C₁₂ alkylene, or C₂-C₁₂ alkenylene;
each L¹ is independently —OC(═O)R¹, —C(═O)OR¹, —OC(═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, —NR^{a}C(═O)OR¹, —SC(═S)R¹, —C(═S)SR¹, —C(═S)R¹, —CH(OH)R¹, —P(═O)(OR^{b})(OR^{c}), —(C₆-C₁₀ arylene)-R¹, -(6- to 10-membered heteroarylene)-R¹, or R¹;
R¹ is C₆-C₂₄ alkyl or C₆-C₂₄ alkenyl;
R^{a} and R^{b} are each independently H, C₁-C₁₂ alkyl, or C₂-C₁₂ alkenyl;
R^{c} is C₁-C₂₄ alkyl or C₂-C₂₄ alkenyl;
R³ is hydrogen, C₁-C₁₂ alkyl, C₂-C₁₂ alkenyl, C₂-C₁₂ alkynyl, C₃-C₈ cycloalkyl, C₃-C₈ cycloalkenyl, C₃-C₈ cycloalkynyl, 4- to 8-membered heterocyclyl, C₆-C₁₀ aryl, or 5- to 10-membered heteroaryl; or R³, G¹ or part of G¹, together with the nitrogen to which they are attached form a cyclic moiety;
x is 0, 1, or 2;
n is 1 or 2; and
Z is —OH or halogen;
wherein each alkyl, alkenyl, alkynyl, cycloalkyl, cycloalkenyl, cycloalkynyl, heterocyclyl, aryl, heteroaryl, alkylene, alkenylene, cycloalkylene, cycloalkenylene, cycloalkynylene, heterocyclylene, arylene, heteroarylene, and cyclic moiety is independently optionally substituted.

32. A composition comprising the compound of claim 1, and a therapeutic or prophylactic agent.

33-49. (canceled)

50. A lipid nanoparticle comprising the compound of claim 1.

51. A pharmaceutical composition comprising the compound of claim 1, and a pharmaceutically acceptable excipient or diluent.

52. A method of delivering a therapeutic or prophylactic agent to a mammalian cell or organ, comprising contacting the mammalian cell or organ with the composition of claim 32.

53. A composition comprising the compound of claim 31, and a therapeutic or prophylactic agent.

54. A lipid nanoparticle comprising the compound of claim 31.

55. A pharmaceutical composition comprising the compound of claim 31, and a pharmaceutically acceptable excipient or diluent.

56. A method of delivering a therapeutic or prophylactic agent to a mammalian cell or organ, comprising contacting the mammalian cell or organ with the composition of claim 53.
